(19) United States
(12) Patent Application Publication
(10) Pub. No.: US 2009/0215042 A1
(43) Pub. Date: Aug. 27, 2009

(54) NOVEL NUCLEOTIDE AND AMINO ACID SEQUENCES, AND ASSAYS AND METHODS OF USE THEREOF FOR DIAGNOSIS

(75) Inventors: Osnat Sella-Tavor, Kfar Kish (IL); Sarah Pollock, Tel Aviv (IL); Gad S. Cojocaru, Ramat-Hasharon (IL); Amit Novik, Tel-Mond (IL); Lily Bazak, Givatayim (IL); Elena Tsypkin, Tel Aviv (IL); Shira Wallach, Hod Hasharon (IL); Shirley Sameah-Greenwald, Kfar Saba (IL)

Correspondence Address: WINSTON & STRAWN LLP;PATENT DEPARTMENT, 1700 K STREET, N.W., WASHINGTON, DC, 20006, US

(73) Assignee: Compugen Ltd.

(21) Appl. No.: 11/913,171

(22) PCT Filed: Jun. 8, 2006
(86) PCT No.: PCT/IL06/00676

§ 371 Date: May 2, 2008

Related U.S. Application Data
(60) Provisional application No. 60/688,320, filed on Jun. 8, 2005.
(60) Provisional application No. 60/699,427, filed on Jul. 15, 2005.
(60) Provisional application No. 60/704,414, filed on Aug. 2, 2005.

(51) Int. Cl. C12Q 1/68; C07H 21/00; C12N 15/64; C12N 5/06; G01N 33/53; C12P 21/00; C07K 16/18

(52) U.S. Cl. 435/6; 536/23.1; 435/320.1; 435/325; 435/69.1; 530/387.9; 435/7.1; 435/7.92

(57) ABSTRACT

Novel splice variants, amino acid sequences and nucleotide sequences thereof, and methods of using same.

### FIELD OF THE INVENTION

The present invention is related to novel nucleotide and protein sequences, and assays and methods of use thereof.

### BACKGROUND OF THE INVENTION

Diagnostic markers are important for early diagnosis of many diseases, as well as predicting response to treatment, monitoring treatment and determining prognosis of such diseases.

Serum markers are examples of such diagnostic markers and are used for diagnosis of many different diseases. Such serum markers typically encompass secreted proteins and/or peptides; however, some serum markers may be released to the blood upon tissue lysis, such as from myocardial infarction (for example Troponin-I). Serum markers can also be used as risk factors for disease (for example base-line levels of CRP, as a predictor of cardiovascular disease), to monitor disease activity and progression (for example, determination of CRP levels to monitor acute phase inflammatory response) and to predict and monitor drug response (for example, as shedded fragments of the protein Erb-B2).

Immunohistochemistry (IHC) is the study of distribution of an antigen of choice in a sample based on specific antibody-antigen binding, typically on tissue slices. The antibody features a label which can be detected, for example as a stain which is detectable under a microscope. The tissue slices are prepared by being fixed. IHC is therefore particularly suitable for antibody-antigen reactions that are not disturbed or destroyed by the process of fixing the tissue slices.

IHC permits determining the localization of binding, and hence mapping of the presence of the antigen within the tissue and even within different compartments in the cell. Such mapping can provide useful diagnostic information, including:

1) the histological type of the tissue sample
2) the presence of specific cell types within the sample
3) information on the physiological and/or pathological state of cells (e.g. which phase of the cell-cycle they are in)
4) the presence of disease related changes within the sample
5) differentiation between different specific disease subtypes where it is already known the tissue is of disease state (for example, the differentiation between different tumor types when it is already known the sample was taken from cancerous tissue).

IHC information is valuable for more than diagnosis. It can also be used to determine prognosis and therapy treatment (as in the case of HER-2 in breast cancer) and monitor disease.

IHC protein markers could be from any cellular location. Most often these markers are membrane proteins but secreted proteins or intracellular proteins (including intranuclear) can be used as an IHC marker too.

IHC has at least two major disadvantages. It is performed on tissue samples and therefore a tissue sample has to be collected from the patient, which most often requires invasive procedures like biopsy associated with pain, discomfort, hospitalization and risk of infection. In addition, the interpretation of the result is observer dependant and therefore subjective. There is no measured value but rather only an estimation (on a scale of 1-4) of how prevalent the antigen on target is.

### SUMMARY OF THE INVENTION

The present invention provides, in different embodiments, many novel amino acid and nucleic acid sequences, which may optionally be used as diagnostic markers.

For example, the present invention provides a number of different variants of known serum proteins, which may optionally be used as diagnostic markers, preferably as serum markers, or optionally as IHC markers. The present invention therefore overcomes the many deficiencies of the background art with regard to the need to obtain tissue samples and subjective interpretations of results. For example, serum markers require only a simple blood test and their result is typically a scientifically measured number. As IHC markers, the variants of the present invention may also provide different and/or better measurement parameters for various diseases and/or pathological conditions. The markers presented in the present invention can also potentially be used for in-vivo imaging applications.

The present invention also provides a number of different variants of known IHC proteins, which may optionally be used as diagnostic markers, preferably as serum markers, or optionally as IHC markers. The present invention therefore overcomes the many deficiencies of the background art with regard to the need to obtain tissue samples and subjective interpretations of results. For example, serum markers require only a simple blood test and their result is typically a scientifically measured number. As IHC markers, the variants of the present invention may also provide different and/or better measurement parameters for various diseases and/or pathological conditions.

Other variants are also provided by the present invention as described in greater detail below.

The diseases for which such variants may be useful diagnostic markers are described in greater detail below for each of the variants. The variants themselves are described by “cluster” or by gene, as these variants are splice variants of known proteins. Therefore, a “marker-detectable disease” refers to a disease that may be detected by a particular marker, with regard to the description of such diseases below. The markers of the present invention, alone or in combination, show a high degree of differential detection between disease and non-disease states.

The present invention therefore also relates to diagnostic assays for disease detection optionally and preferably in a biological sample taken from a subject (patient), which is more preferably some type of body fluid or secretion including but not limited to seminal plasma, blood, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, broncho alveolar lavage, lavage of the reproductive system and/or lavage of any other part of the body or system in the body, and stool or a tissue sample. The term may also optionally encompass samples of in vivo cell culture constituents. The sample can optionally be diluted with a suitable eluant before contacting the sample to an antibody and/or performing any other diagnostic assay.

An isolated chimeric polypeptide encoding for N56180_P2 (SEQ ID NO:84), comprising a first amino acid sequence being at least about 90% or preferably at least about 95% homologous to amino acids 1-203 of CAQ2_HUMAN (SEQ ID NO:83), which also corresponds to amino acids 1-203 of N56180_P2 (SEQ ID NO:84), and a second amino acid sequence being at least 70%, optionally at least 80%, preferably at least 85%, more preferably at least 90% and most preferably at least 95% homologous to a polypeptide having the sequence LWLTPVIPTLWEADGGGLHEPWSWRPAWATWLQRNYL (SEQ ID NO: 628) corresponding to amino acids 204-240 of N56180_P2 (SEQ ID NO:84), wherein said first amino acid sequence and second amino acid sequence are contiguous and in a sequential order.

An isolated polypeptide encoding for a tail of N56180_P2 (SEQ ID NO:84), comprising a polypeptide being at least 70%, optionally at least about 80%, preferably at least about 85%, more preferably at least about 90% and most preferably at least about 95% homologous to the sequence LWLTPVIPTLWEADGGGLHEPWSWRPAWATWLQRNYL (SEQ ID NO: 628) in N56180_P2 (SEQ ID NO:84).

An isolated chimeric polypeptide encoding for N56180_P4 (SEQ ID NO:85), comprising a first amino acid sequence being at least about 90% or preferably at least about 95% homologous to amino acids 1-78 of CAQ2 HUMAN (SEQ ID NO:83), which also corresponds to amino acids 1-78 of N56180_P4 (SEQ ID NO:85), a second amino acid sequence being at least 70%, optionally at least 80%, preferably at least 85%, more preferably at least 90% and most preferably at least 95% homologous to a polypeptide having the sequence HWQISQWWLHFQTPREEGKMKLLELSESADGAAWKRWGGNSNTHRIQ (SEQ ID NO: 629) corresponding to amino acids 79-125 of N56180_P4 (SEQ ID NO:85), and a third amino acid sequence being at least about 90% or preferably at least about 95% homologous amino acids 79-399 of CAQ2_HUMAN (SEQ ID NO:83), which also corresponds to amino acids 126-446 of N56180_P4 (SEQ ID NO:85), wherein said first amino acid sequence, second amino acid sequence and third amino acid sequence are contiguous and in a sequential order.

An isolated polypeptide encoding for an edge portion of N56180_P4 (SEQ ID NO:85), comprising an amino acid sequence being at least 70%, optionally at least about 80%, preferably at least about 85%, more preferably at least about 90% and most preferably at least about 95% homologous to the sequence encoding for HWQISQWWLHFQTPREEGKMKLLELSESADGAAWKRWGGNSNTHRIQ (SEQ ID NO: 629), corresponding to N56180_P4 (SEQ ID NO:85).

An isolated chimeric polypeptide encoding for N56180_P5 (SEQ ID NO:86), comprising a first amino acid sequence being at least about 90% or preferably at least about 95% homologous amino acids 1-140 of CAQ2_HUMAN (SEQ ID NO:83), which also corresponds to amino acids 1-140 of N56180_P5 (SEQ ID NO:86), and a second amino acid sequence being at least about 90% or preferably at least about 95% homologous to amino acids 203-399 of CAQ2_HUMAN (SEQ ID NO:83), which also corresponds to amino acids 141-337 of N56180_P5 (SEQ ID NO:86), wherein said first amino acid sequence and second amino acid sequence are contiguous and in a sequential order.

An isolated chimeric polypeptide encoding for an edge portion of N56180_P5 (SEQ ID NO:86), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise DV, having a structure as follows: a sequence starting from any of amino acid numbers 140−x to 140; and ending at any of amino acid numbers 141+((n−2)−x), in which x varies from 0 to n−2.

An isolated chimeric polypeptide encoding for N56180_P6 (SEQ ID NO:87), comprising a first amino acid sequence being at least 70%, optionally at least 80%, preferably at least 85%, more preferably at least 90% and most preferably at least 95% homologous to a polypeptide having the sequence NETEAEQSYV (SEQ ID NO: 631) corresponding to amino acids 1-10 of N56180_P6 (SEQ ID NO:87), a second amino acid sequence being at least about 90% or preferably at least about 95% homologous to amino acids 18-106 of CAQ2_HUMAN (SEQ ID NO:83), which also corresponds to amino acids 11-99 of N56180_P6 (SEQ ID NO:87), a third amino acid sequence bridging amino acid sequence comprising of D, and a fourth amino acid sequence being at least about 90% or preferably at least about 95% homologous amino acids 179-399 of CAQ2_HUMAN (SEQ ID NO:83), which also corresponds to amino acids 101-321 of N56180_P6 (SEQ ID NO:87), wherein said first amino acid sequence, second amino acid sequence, third amino acid sequence and fourth amino acid sequence are contiguous and in a sequential order.

An isolated polypeptide encoding for a head of N56180_P6 (SEQ ID NO:87), comprising a polypeptide being at least 70%, optionally at least about 80%, preferably at least about 85%, more preferably at least about 90% and most preferably at least about 95% homologous to the sequence NETEAEQSYV (SEQ ID NO: 631) of N56180_P6 (SEQ ID NO:87).

An isolated polypeptide encoding for an edge portion of N56180_P6 (SEQ ID NO:87), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise LDY having a structure as follows (numbering according to N56180_P6 (SEQ ID NO:87)): a sequence starting from any of amino acid numbers 99−x to 99; and ending at any of amino acid numbers 101+((n−2)−x), in which x varies from 0 to n−2.

An isolated chimeric polypeptide encoding for N56180_P7 (SEQ ID NO:88), comprising a first amino acid sequence being at least 70%, optionally at least 80%, preferably at least 85%, more preferably at least 90% and most preferably at least 95% homologous to a polypeptide having the sequence MSSWLSAGSPSSLSV (SEQ ID NO: 632) corresponding to amino acids 1-15 of N56180_P7 (SEQ ID NO:88), and a second amino acid sequence being at least about 90% or preferably at least about 95% homologous amino acids 203-399 of CAQ2_HUMAN (SEQ ID NO:83), which also corresponds to amino acids 16-212 of N56180_P7 (SEQ ID NO:88), wherein said first amino acid sequence and second amino acid sequence are contiguous and in a sequential order.

An isolated polypeptide encoding for a head of N56180_P7 (SEQ ID NO:88), comprising a polypeptide being at least 70%, optionally at least about 80%, preferably at least about 85%, more preferably at least about 90% and most preferably at least about 95% homologous to the sequence MSSWLSAGSPSSLSV (SEQ ID NO: 632) of N56180_P7 (SEQ ID NO:88).

An isolated chimeric polypeptide encoding for N56180_P8 (SEQ ID NO:89), comprising a first amino acid sequence being at least 70%, optionally at least 80%, preferably at least 85%, more preferably at least 90% and most preferably at least 95% homologous to a polypeptide having the sequence MCRGYSTLLNPVS (SEQ ID NO: 633) corresponding to amino acids 1-13 of N56180_P8 (SEQ ID NO:89), and a second amino acid sequence being at least about 90% or preferably at least about 95% homologous to amino acids 280-399 of CAQ2_HUMAN (SEQ ID NO:83), which also corresponds to amino acids 14-133 of N56180_P8 (SEQ ID NO:89), wherein said first amino acid sequence and second amino acid sequence are contiguous and in a sequential order.

An isolated polypeptide encoding for a head of N56180_P8 (SEQ ID NO:89), comprising a polypeptide being at least 70%, optionally at least about 80%, preferably at least about 85%, more preferably at least about 90% and most preferably at least about 95% homologous to the sequence MCRGYSTLLNPVS (SEQ ID NO: 633) of N56180_P8 (SEQ ID NO:89).

An isolated chimeric polypeptide encoding for N56180_P9 (SEQ ID NO:90), comprising a first amino acid sequence being at least about 90% or preferably at least about 95% homologous to amino acids 1-246 of CAQ2_HUMAN (SEQ ID NO:83), which also corresponds to amino acids 1-246 of N56180_P9 (SEQ ID NO:90), and a second amino acid sequence being at least 70%, optionally at least 80%, preferably at least 85%, more preferably at least 90% and most preferably at least 95% homologous to a polypeptide having the sequence SRNWTQ (SEQ ID NO: 634) corresponding to amino acids 247-252 of N56180_P9 (SEQ ID NO:90), wherein said first amino acid sequence and second amino acid sequence are contiguous and in a sequential order.

An isolated polypeptide encoding for a tail of N56180_P9 (SEQ ID NO:90), comprising a polypeptide being at least 70%, optionally at least about 80%, preferably at least about 85%, more preferably at least about 90% and most preferably at least about 95% homologous to the sequence SRNWTQ (SEQ ID NO: 634) in N56180_P9 (SEQ ID NO:90).

An isolated chimeric polypeptide encoding for S67314_PEA_{—}1_P4 (SEQ ID NO:114), comprising a first amino acid sequence being at least about 90% or preferably at least about 95% homologous to amino acids 1-116 of FABH_HUMAN_V1 (SEQ ID NO:113), which also corresponds to amino acids 1-116 of S67314_PEA_{—}1_P4 (SEQ ID NO:114), and a second amino acid sequence being at least 70%, optionally at least 80%, preferably at least 85%, more preferably at least 90% and most preferably at least 95% homologous to a polypeptide having the sequence VRWATLELYLIGYYYCSFSQACSKKPSPPLRAVEAGTREWLWVRVVSGGNFLCSGFGLTQAGTQI LPYRLHDCGQITFSKCNCKTGINNTNLVGLLGSL (SEQ ID NO: 635) corresponding to amino acids 117-215 of S67314_PEA_{—}1_P4 (SEQ ID NO:114), wherein said first amino acid sequence and second amino acid sequence are contiguous and in a sequential order.

An isolated polypeptide encoding for a tail of S67314_PEA_{—}1_P4 (SEQ ID NO:114), comprising a polypeptide being at least 70%, optionally at least about 80%, preferably at least about 85%, more preferably at least about 90% and most preferably at least about 95% homologous to the sequence VRWATLELYLIGYYYCSFSQACSKKPSPPLRAVEAGTREWLWVRVVSGGNFLCSGFGLTQAGTQI LPYRLHDCGQITFSKCNCKTGINNTNLVGLLGSL (SEQ ID NO: 635) in S67314_PEA_{—}1_P4 (SEQ ID NO:114).

An isolated chimeric polypeptide encoding for S67314_PEA_{—}1_P5 (SEQ ID NO:115), comprising a first amino acid sequence being at least about 90% or preferably at least about 95% homologous amino acids 1-116 of FABH_HUMAN_V1 (SEQ ID NO:113), which also corresponds to amino acids 1-116 of S67314_PEA_{—}1_P5 (SEQ ID NO:115), and a second amino acid sequence being at least 70%, optionally at least 80%, preferably at least 85%, more preferably at least 90% and most preferably at least 95% homologous to a polypeptide having the sequence DVLTAWPSIYRRQVKVLREDEITILPWHLQWSREKATKLLRPTLPSYNNHGWEELRVGKSIV (SEQ ID NO: 636) corresponding to amino acids 117-178 of S67314_PEA_{—}1_P5 (SEQ ID NO:115), wherein said first amino acid sequence and second amino acid sequence are contiguous and in a sequential order.

An isolated polypeptide encoding for a tail of S67314_PEA_{—}1_P5 (SEQ ID NO:115), comprising a polypeptide being at least 70%, optionally at least about 80%, preferably at least about 85%, more preferably at least about 90% and most preferably at least about 95% homologous to the sequence DVLTAWPSIYRRQVKVLREDEITILPWHLQWSREKATKLLRPTLPSYNNHGWEELRVGKSIV (SEQ ID NO: 636) in S67314_PEA_{—}1_P5 (SEQ ID NO:115).

An isolated chimeric polypeptide encoding for S67314_PEA_{—}1_P6 (SEQ ID NO:116), comprising a first amino acid sequence being at least about 90% or preferably at least about 95% homologous amino acids 1-116 of FABH_HUMAN_V1 (SEQ ID NO:113), which also corresponds to amino acids 1-116 of S67314_PEA_{—}1_P6 (SEQ ID NO:116), and a second amino acid sequence being at least 70%, optionally at least 80%, preferably at least 85%, more preferably at least 90% and most preferably at least 95% homologous to a polypeptide having the sequence MEKLQLRNVK (SEQ ID NO: 637) corresponding to amino acids 117-126 of S67314_PEA_{—}1_P6 (SEQ ID NO:116), wherein said first amino acid sequence and second amino acid sequence are contiguous and in a sequential order.

An isolated polypeptide encoding for a tail of S67314_PEA_{—}1_P6 (SEQ ID NO:116), comprising a polypeptide being at least 70%, optionally at least about 80%, preferably at least about 85%, more preferably at least about 90% and most preferably at least about 95% homologous to the sequence MEKLQLRNVK (SEQ ID NO: 637) in S67314_PEA_{—}1_P6 (SEQ ID NO:116).

An isolated chimeric polypeptide encoding for S67314_PEA_{—}1_P7 (SEQ ID NO:117), comprising a first amino acid sequence being at least about 90% or preferably at least about 95% homologous to amino acids 1-24 of FABH_HUMAN_V1 (SEQ ID NO:113), which also corresponds to amino acids 1-24 of S67314_PEA_{—}1_P7 (SEQ ID NO:117), a second amino acid sequence being at least 70%, optionally at least 80%, preferably at least 85%, more preferably at least 90% and most preferably at least 95% homologous to a polypeptide having the sequence AHILITFPLPS (SEQ ID NO: 638) corresponding to amino acids 25-35 of S67314_PEA_{—}1_P7 (SEQ ID NO:117), and a third amino acid sequence being at least about 90% or preferably at least about 95% homologous amino acids 25-133 of FABH_HUMAN_V1 (SEQ ID NO:113), which also corresponds to amino acids 36-144 of S67314_PEA_{—}1_P7 (SEQ ID NO:117), wherein said first amino acid sequence, second amino acid sequence and third amino acid sequence are contiguous and in a sequential order.

An isolated polypeptide encoding for an edge portion of S67314_PEA_{—}1_P7 (SEQ ID NO:117), comprising an amino acid sequence being at least 70%, optionally at least about 80%, preferably at least about 85%, more preferably at least about 90% and most preferably at least about 95% homologous to the sequence encoding for AHILITFPLPS (SEQ ID NO: 638), corresponding to S67314_PEA_{—}1_P7 (SEQ ID NO:117).

An isolated chimeric polypeptide encoding for HUMNATPEP_PEA_{—}1_P2 (SEQ ID NO:139), comprising a first amino acid sequence being at least about 90% or preferably at least about 95% homologous to amino acids 1-129 of ANFB_HUMAN (SEQ ID NO:138), which also corresponds to amino acids 1-129 of HUMNATPEP_PEA_{—}1_P2 (SEQ ID NO:139), and a second amino acid sequence being at least 70%, optionally at least 80%, preferably at least 85%, more preferably at least 90% and most preferably at least 95% homologous to a polypeptide having the sequence GKHPLPPRPPSPIPVCDTVRVTLGFVVSGNHTL (SEQ ID NO: 640) corresponding to amino acids 130-162 of HUMNATPEP_PEA_{—}1_P2 (SEQ ID NO:139), wherein said first amino acid sequence and second amino acid sequence are contiguous and in a sequential order.

An isolated polypeptide encoding for a tail of HUMNATPEP_PEA_{—}1_P2 (SEQ ID NO:139), comprising a polypeptide being at least 70%, optionally at least about 80%, preferably at least about 85%, more preferably at least about 90% and most preferably at least about 95% homologous to the sequence GKHPLPPRPPSPIPVCDTVRVTLGFVVSGNHTL (SEQ ID NO: 640) in HUMNATPEP_PEA_{—}1_P2 (SEQ ID NO:139).

An isolated chimeric polypeptide encoding for HUMNATPEP_PEA_{—}1_P3 (SEQ ID NO:140), comprising a first amino acid sequence being at least about 90% or preferably at least about 95% homologous to amino acids 1-44 of ANFB_HUMAN (SEQ ID NO:138), which also corresponds to amino acids 1-44 of HUMNATPEP_PEA_{—}1_P3 (SEQ ID NO:140), and a second amino acid sequence being at least 70%, optionally at least 80%, preferably at least 85%, more preferably at least 90% and most preferably at least 95% homologous to a polypeptide having the sequence VRAEGSSGGLDSSNERVLTCCPKRPSSFLWN (SEQ ID NO: 641) corresponding to amino acids 45-75 of HUMNATPEP_PEA_{—}1_P3 (SEQ ID NO:140), wherein said first amino acid sequence and second amino acid sequence are contiguous and in a sequential order.

An isolated polypeptide encoding for a tail of HUMNATPEP_PEA_{—}1_P3 (SEQ ID NO:140), comprising a polypeptide being at least 70%, optionally at least about 80%, preferably at least about 85%, more preferably at least about 90% and most preferably at least about 95% homologous to the sequence VRAEGSSGGLDSSNERVLTCCPKRPSSFLWN (SEQ ID NO: 641) in HUMNATPEP_PEA_{—}1_P3 (SEQ ID NO:140).

An isolated chimeric polypeptide encoding for HUMNATPEP_PEA_{—}1_P7 (SEQ ID NO:141), comprising a first amino acid sequence being at least about 90% or preferably at least about 95% homologous to amino acids 93-134 of ANFB_HUMAN (SEQ ID NO:138), which also corresponds to amino acids 1-42 of HUMNATPEP_PEA_{—}1_P7 (SEQ ID NO:141).

An isolated chimeric polypeptide encoding for HUMCDDANF_PEA_{—}1_P6 (SEQ ID NO:165), comprising a first amino acid sequence being at least about 90% or preferably at least about 95% homologous to amino acids 1-150 of ANF_HUMAN (SEQ ID NO:164), which also corresponds to amino acids 1-150 of HUMCDDANF_PEA_{—}1_P6 (SEQ ID NO:165), and a second amino acid sequence being at least 70%, optionally at least 80%, preferably at least 85%, more preferably at least 90% and most preferably at least 95% homologous to a polypeptide having the sequence VRGTGDGNGMGWTLLGDTFSRKGTNAEAHSLSSFCPNTQSAPWVSGHAIYCP (SEQ ID NO: 642) corresponding to amino acids 151-202 of HUMCDDANF_PEA_{—}1_P6 (SEQ ID NO:165), wherein said first amino acid sequence and second amino acid sequence are contiguous and in a sequential order.

An isolated polypeptide encoding for a tail of HUMCDDANF_PEA_{—}1_P6 (SEQ ID NO:165), comprising a polypeptide being at least 70%, optionally at least about 80%, preferably at least about 85%, more preferably at least about 90% and most preferably at least about 95% homologous to the sequence VRGTGDGNGMGWTLLGDTFSRKGTNAEAHSLSSFCPNTQSAPWVSGHAIYCP (SEQ ID NO: 642) in HUMCDDANF_PEA_{—}1_P6 (SEQ ID NO:165).

An isolated chimeric polypeptide encoding for HUMCDDANF_PEA_{—}1_P9 (SEQ ID NO:166), comprising a first amino acid sequence being at least about 90% or preferably at least about 95% homologous to amino acids 1-41 of ANF_HUMAN (SEQ ID NO:164), which also corresponds to amino acids 1-41 of HUMCDDANF_PEA_{—}1_P9 (SEQ ID NO:166), and a second amino acid sequence being at least 70%, optionally at least 80%, preferably at least 85%, more preferably at least 90% and most preferably at least 95% homologous to a polypeptide having the sequence VGPGKRVQSGARGLSDAVLTPLDFLQVSEVYPFPCIFLF (SEQ ID NO: 643) corresponding to amino acids 42-80 of HUMCDDANF_PEA_{—}1_P9 (SEQ ID NO:166), wherein said first amino acid sequence and second amino acid sequence are contiguous and in a sequential order.

An isolated polypeptide encoding for a tail of HUMCDDANF_PEA_{—}1_P9 (SEQ ID NO:166), comprising a polypeptide being at least 70%, optionally at least about 80%, preferably at least about 85%, more preferably at least about 90% and most preferably at least about 95% homologous to the sequence VGPGKRVQSGARGLSDAVLTPLDFLQVSEVYPFPCIFLF (SEQ ID NO: 643) in HUMCDDANF_PEA_{—}1_P9 (SEQ ID NO:166).

An isolated chimeric polypeptide encoding for HSACMHCP_PEA_{—}1_P2 (SEQ ID NO:239), comprising a first amino acid sequence being at least about 90% or preferably at least about 95% homologous to amino acids 1-1855 of MYH6_HUMAN_V1 (SEQ ID NO:236), which also corresponds to amino acids 1-1855 of HSACMHCP_PEA_{—}1_P2 (SEQ ID NO:239), and a second amino acid sequence being at least 70%, optionally at least 80%, preferably at least 85%, more preferably at least 90% and most preferably at least 95% homologous to a polypeptide having the sequence VRRTPDTGSRCGSFFSGPTAPPSQGSSHLLLEMLLVDLTFFSRSAVSLT (SEQ ID NO: 644) corresponding to amino acids 1856-1904 of HSACMHCP_PEA_{—}1_P2 (SEQ ID NO:239), wherein said first amino acid sequence and second amino acid sequence are contiguous and in a sequential order.

An isolated polypeptide encoding for a tail of HSACMHCP_PEA_{—}1_P2 (SEQ ID NO:239), comprising a polypeptide being at least 70%, optionally at least about 80%, preferably at least about 85%, more preferably at least about 90% and most preferably at least about 95% homologous to the sequence VRRTPDTGSRCGSFFSGPTAPPSQGSSHLLLEMLLVDLTFFSRSAVSLT (SEQ ID NO: 644) in HSACMHCP_PEA_{—}1_P2 (SEQ ID NO:239).

An isolated chimeric polypeptide encoding for HSACMHCP_PEA_{—}1_P2 (SEQ ID NO:239), comprising a first amino acid sequence being at least about 90% or preferably at least about 95% homologous to amino acids 1-1855 of MYH6_HUMAN_V1 (SEQ ID NO:236), which also corresponds to amino acids 1-1855 of HSACMHCP_PEA_{—}1_P2 (SEQ ID NO:239), and a second amino acid sequence being at least 70%, optionally at least 80%, preferably at least 85%, more preferably at least 90% and most preferably at least 95% homologous to a polypeptide having the sequence VRRTPDTGSRCGSFFSGPTAPPSQGSSHLLLEMLLVDLTFFSRSAVSLT (SEQ ID NO: 644) corresponding to amino acids 1856-1904 of HSACMHCP_PEA_{—}1_P2 (SEQ ID NO:239), wherein said first amino acid sequence and second amino acid sequence are contiguous and in a sequential order.

An isolated polypeptide encoding for a tail of HSACMHCP_PEA_{—}1_P2 (SEQ ID NO:239), comprising a polypeptide being at least 70%, optionally at least about 80%, preferably at least about 85%, more preferably at least about 90% and most preferably at least about 95% homologous to the sequence VRRTPDTGSRCGSFFSGPTAPPSQGSSHLLLEMLLVDLTFFSRSAVSLT (SEQ ID NO: 644) in HSACMHCP_PEA_{—}1_P2 (SEQ ID NO:239).

An isolated chimeric polypeptide encoding for HSACMHCP_PEA_{—}1_P3 (SEQ ID NO:240), comprising a first amino acid sequence being at least about 90% or preferably at least about 95% homologous to amino acids 1-1326 of MYH6_HUMAN_V2 (SEQ ID NO:237), which also corresponds to amino acids 1-1326 of HSACMHCP_PEA_{—}1_P3 (SEQ ID NO:240), and a second amino acid sequence being at least 70%, optionally at least 80%, preferably at least 85%, more preferably at least 90% and most preferably at least 95% homologous to a polypeptide having the sequence VRPSGEGGQA (SEQ ID NO: 645) corresponding to amino acids 1327-1336 of HSACMHCP_PEA_{—}1_P3 (SEQ ID NO:240), wherein said first amino acid sequence and second amino acid sequence are contiguous and in a sequential order.

An isolated polypeptide encoding for a tail of HSACMHCP_PEA_{—}1_P3 (SEQ ID NO:240), comprising a polypeptide being at least 70%, optionally at least about 80%, preferably at least about 85%, more preferably at least about 90% and most preferably at least about 95% homologous to the sequence VRPSGEGGQA (SEQ ID NO: 645) in HSACMHCP_PEA_{—}1_P3 (SEQ ID NO:240).

An isolated chimeric polypeptide encoding for HSACMHCP_PEA_{—}1_P4 (SEQ ID NO:241), comprising a first amino acid sequence being at least about 90% or preferably at least about 95% homologous to amino acids 1-1508 of MYH6_HUMAN_V2 (SEQ ID NO:237), which also corresponds to amino acids 1-1508 of HSACMHCP_PEA_{—}1_P4 (SEQ ID NO:241), and a second amino acid sequence being at least 70%, optionally at least 80%, preferably at least 85%, more preferably at least 90% and most preferably at least 95% homologous to a polypeptide having the sequence GVLGVQEARDELVGGRAMQGQGEHRL (SEQ ID NO: 646) corresponding to amino acids 1509-1534 of HSACMHCP_PEA_{—}1_P4 (SEQ ID NO:241), wherein said first amino acid sequence and second amino acid sequence are contiguous and in a sequential order.

An isolated polypeptide encoding for a tail of HSACMHCP_PEA_{—}1_P4 (SEQ ID NO:241), comprising a polypeptide being at least 70%, optionally at least about 80%, preferably at least about 85%, more preferably at least about 90% and most preferably at least about 95% homologous to the sequence GVLGVQEARDELVGGRAMQGQGEHRL (SEQ ID NO: 646) in HSACMHCP_PEA_{—}1_P4 (SEQ ID NO:241).

An isolated chimeric polypeptide encoding for HSACMHCP_PEA_{—}1_P6 (SEQ ID NO:242), comprising a first amino acid sequence being at least about 90% or preferably at least about 95% homologous to amino acids 1-1763 of MYH6_HUMAN_V1 (SEQ ID NO:236), which also corresponds to amino acids 1-1763 of HSACMHCP_PEA_{—}1_P6 (SEQ ID NO:242), and a second amino acid sequence being at least 70%, optionally at least 80%, preferably at least 85%, more preferably at least 90% and most preferably at least 95% homologous to a polypeptide having the sequence VSDRPPSASPKDRNKALGPGQATVL (SEQ ID NO: 647) corresponding to amino acids 1764-1788 of HSACMHCP_PEA_{—}1_P6 (SEQ ID NO:242), wherein said first amino acid sequence and second amino acid sequence are contiguous and in a sequential order.

An isolated polypeptide encoding for a tail of HSACMHCP_PEA_{—}1_P6 (SEQ ID NO:242), comprising a polypeptide being at least 70%, optionally at least about 80%, preferably at least about 85%, more preferably at least about 90% and most preferably at least about 95% homologous to the sequence VSDRPPSASPKDRNKALGPGQATVL (SEQ ID NO: 647) in HSACMHCP_PEA_{—}1_P6 (SEQ ID NO:242).

An isolated chimeric polypeptide encoding for HSACMHCP_PEA_{—}1_P12 (SEQ ID NO:243), comprising a first amino acid sequence being at least 70%, optionally at least 80%, preferably at least 85%, more preferably at least 90% and most preferably at least 95% homologous to a polypeptide having the sequence MGLWKPGSVLSDSLFASSPCPQ (SEQ ID NO: 648) corresponding to amino acids 1-22 of HSACMHCP_PEA_{—}1_P12 (SEQ ID NO:243), and a second amino acid sequence being at least about 90% or preferably at least about 95% homologous to amino acids 528-1939 of MYH6_HUMAN_V3 (SEQ ID NO:238), which also corresponds to amino acids 23-1434 of HSACMHCP_PEA_{—}1_P12 (SEQ ID NO:243), wherein said first amino acid sequence and second amino acid sequence are contiguous and in a sequential order.

An isolated polypeptide encoding for a head of HSACMHCP_PEA_{—}1_P12 (SEQ ID NO:243), comprising a polypeptide being at least 70%, optionally at least about 80%, preferably at least about 85%, more preferably at least about 90% and most preferably at least about 95% homologous to the sequence MGLWKPGSVLSDSLFASSPCPQ (SEQ ID NO: 648) of HSACMHCP_PEA_{—}1_P12 (SEQ ID NO:243).

An isolated chimeric polypeptide encoding for HSACMHCP_PEA_{—}1_P16 (SEQ ID NO:244), comprising a first amino acid sequence being at least about 90% or preferably at least about 95% homologous to amino acids 1-527 of MYH6_HUMAN_V2 (SEQ ID NO:237), which also corresponds to amino acids 1-527 of HSACMHCP_PEA_{—}1_P16 (SEQ ID NO:244), and a second amino acid sequence being at least 70%, optionally at least 80%, preferably at least 85%, more preferably at least 90% and most preferably at least 95% homologous to a polypeptide having the sequence VPPWPHHLCPLLCHPDKVVAESLLHPRN (SEQ ID NO: 649) corresponding to amino acids 528-555 of HSACMHCP_PEA_{—}1_P16 (SEQ ID NO:244), wherein said first amino acid sequence and second amino acid sequence are contiguous and in a sequential order.

An isolated polypeptide encoding for a tail of HSACMHCP_PEA_{—}1_P16 (SEQ ID NO:244), comprising a polypeptide being at least 70%, optionally at least about 80%, preferably at least about 85%, more preferably at least about 90% and most preferably at least about 95% homologous to the sequence VPPWPHHLCPLLCHPDKVVAESLLHPRN (SEQ ID NO: 649) in HSACMHCP_PEA_{—}1_P16 (SEQ ID NO:244).

An isolated chimeric polypeptide encoding for HSCREACT_PEA_{—}1_P9 (SEQ ID NO:317), comprising a first amino acid sequence being at least about 90% or preferably at least about 95% homologous to amino acids 1-64 of CRP_HUMAN (SEQ ID NO:316), which also corresponds to amino acids 1-64 of HSCREACT_PEA_{—}1_P9 (SEQ ID NO:317), a second amino acid sequence bridging amino acid sequence comprising of H, and a third amino acid sequence being at least about 90% or preferably at least about 95% homologous to amino acids 188-224 of CRP_HUMAN (SEQ ID NO:316), which also corresponds to amino acids 66-102 of HSCREACT_PEA_{—}1_P9 (SEQ ID NO:317), wherein said first amino acid sequence, second amino acid sequence and third amino acid sequence are contiguous and in a sequential order.

An isolated polypeptide encoding for an edge portion of HSCREACT_PEA_{—}1_P9 (SEQ ID NO:317), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise THE having a structure as follows (numbering according to HSCREACT_PEA_{—}1_P9 (SEQ ID NO:317)): a sequence starting from any of amino acid numbers 64−x to 64; and ending at any of amino acid numbers 66+((n−2)−x), in which x varies from 0 to n−2.

An isolated chimeric polypeptide encoding for HSCREACT_PEA_{—}1_P10 (SEQ ID NO:318), comprising a first amino acid sequence being at least about 90% or preferably at least about 95% homologous to amino acids 1-66 of CRP_HUMAN (SEQ ID NO:316), which also corresponds to amino acids 1-66 of HSCREACT_PEA_{—}1_P10 (SEQ ID NO:318).

An isolated chimeric polypeptide encoding for HSCREACT_PEA_{—}1_P12 (SEQ ID NO:319), comprising a first amino acid sequence being at least about 90% or preferably at least about 95% homologous to amino acids 1-66 of CRP_HUMAN (SEQ ID NO:316), which also corresponds to amino acids 1-66 of HSCREACT_PEA_{—}1_P12 (SEQ ID NO:319), and a second amino acid sequence being at least about 90% or preferably at least about 95% homologous to amino acids 200-224 of CRP_HUMAN (SEQ ID NO:316), which also corresponds to amino acids 67-91 of HSCREACT_PEA_{—}1_P12 (SEQ ID NO:319), wherein said first amino acid sequence and second amino acid sequence are contiguous and in a sequential order.

An isolated chimeric polypeptide encoding for an edge portion of HSCREACT_PEA_{—}1_P12 (SEQ ID NO:319), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GP, having a structure as follows: a sequence starting from any of amino acid numbers 66−x to 66; and ending at any of amino acid numbers 67+((n−2)−x), in which x varies from 0 to n−2.

An isolated chimeric polypeptide encoding for HSCREACT_PEA_{—}1_P16 (SEQ ID NO:320), comprising a first amino acid sequence being at least about 90% or preferably at least about 95% homologous to amino acids 1-160 of CRP_HUMAN (SEQ ID NO:316), which also corresponds to amino acids 1-160 of HSCREACT_PEA_{—}1_P16 (SEQ ID NO:320), and a second amino acid sequence being at least 70%, optionally at least 80%, preferably at least 85%, more preferably at least 90% and most preferably at least 95% homologous to a polypeptide having the sequence VSESGHWPGVWFGSRVLIIMS (SEQ ID NO: 650) corresponding to amino acids 161-181 of HSCREACT_PEA_{—}1_P16 (SEQ ID NO:320), wherein said first amino acid sequence and second amino acid sequence are contiguous and in a sequential order.

An isolated polypeptide encoding for a tail of HSCREACT_PEA_{—}1_P16 (SEQ ID NO:320), comprising a polypeptide being at least 70%, optionally at least about 80%, preferably at least about 85%, more preferably at least about 90% and most preferably at least about 95% homologous to the sequence VSESGHWPGVWFGSRVLIIMS (SEQ ID NO: 650) in HSCREACT_PEA_{—}1_P16 (SEQ ID NO:320).

An isolated chimeric polypeptide encoding for HSCREACT_PEA_{—}1_P22 (SEQ ID NO:321), comprising a first amino acid sequence being at least about 90% or preferably at least about 95% homologous to amino acids 1-66 of CRP_HUMAN (SEQ ID NO:316), which also corresponds to amino acids 1-66 of HSCREACT_PEA_{—}1_P22 (SEQ ID NO:321), and a second amino acid sequence being at least 70%, optionally at least 80%, preferably at least 85%, more preferably at least 90% and most preferably at least 95% homologous to a polypeptide having the sequence AFLILWLFWETPPLFHTNLVGL (SEQ ID NO: 651) corresponding to amino acids 67-88 of HSCREACT_PEA_{—}1_P22 (SEQ ID NO:321), wherein said first amino acid sequence and second amino acid sequence are contiguous and in a sequential order.

An isolated polypeptide encoding for a tail of HSCREACT_PEA_{—}1_P22 (SEQ ID NO:321), comprising a polypeptide being at least 70%, optionally at least about 80%, preferably at least about 85%, more preferably at least about 90% and most preferably at least about 95% homologous to the sequence AFLILWLFWETPPLFHTNLVGL (SEQ ID NO: 651) in HSCREACT_PEA_{—}1_P22 (SEQ ID NO:321).

An isolated chimeric polypeptide encoding for HSCREACT_PEA_{—}1_P28 (SEQ ID NO:322), comprising a first amino acid sequence being at least about 90% or preferably at least about 95% homologous to amino acids 1-64 of CRP_HUMAN (SEQ ID NO:316), which also corresponds to amino acids 1-64 of HSCREACT_PEA_{—}1_P28 (SEQ ID NO:322), and a second amino acid sequence being at least 70%, optionally at least 80%, preferably at least 85%, more preferably at least 90% and most preferably at least 95% homologous to a polypeptide having the sequence LLS corresponding to amino acids 65-67 of HSCREACT_PEA_{—}1_P28 (SEQ ID NO:322), wherein said first amino acid sequence and second amino acid sequence are contiguous and in a sequential order.

An isolated chimeric polypeptide encoding for HSSTROL3_PEA_{—}1_P4 (SEQ ID NO:364), comprising a first amino acid sequence being at least about 90% or preferably at least about 95% homologous to amino acids 1-163 of MMP11_HUMAN (SEQ ID NO:363), which also corresponds to amino acids 1-163 of HSSTROL3_PEA_{—}1_P4 (SEQ ID NO:364), a bridging amino acid H corresponding to amino acid 164 of HSSTROL3_PEA_{—}1_P4 (SEQ ID NO:364), a second amino acid sequence being at least about 90% or preferably at least about 95% homologous to amino acids 165-445 of MMP11_HUMAN (SEQ ID NO:363), which also corresponds to amino acids 165-445 of HSSTROL3_PEA_{—}1_P4 (SEQ ID NO:364), and a third amino acid sequence being at least 70%, optionally at least 80%, preferably at least 85%, more preferably at least 90% and most preferably at least 95% homologous to a polypeptide having the sequence ALGVRQLVGGGHSSRFSHLVVAGLPHACHRKSGSSSQVLCPEPSALLSVAG (SEQ ID NO: 652) corresponding to amino acids 446-496 of HSSTROL3_PEA_{—}1_P4 (SEQ ID NO:364), wherein said first amino acid sequence, bridging amino acid, second amino acid sequence and third amino acid sequence are contiguous and in a sequential order.

An isolated polypeptide encoding for a tail of HSSTROL3_PEA_{—}1_P4 (SEQ ID NO:364), comprising a polypeptide being at least 70%, optionally at least about 80%, preferably at least about 85%, more preferably at least about 90% and most preferably at least about 95% homologous to the sequence ALGVRQLVGGGHSSRFSHLVVAGLPHACHRKSGSSSQVLCPEPSALLSVAG (SEQ ID NO: 652) in HSSTROL3_PEA_{—}1_P4 (SEQ ID NO:364).

An isolated chimeric polypeptide encoding for HSSTROL3_PEA_{—}1_P5 (SEQ ID NO:365), comprising a first amino acid sequence being at least about 90% or preferably at least about 95% homologous amino acids 1-163 of MMP11_HUMAN (SEQ ID NO:363), which also corresponds to amino acids 1-163 of HSSTROL3_PEA_{—}1_P5 (SEQ ID NO:365), a bridging amino acid H corresponding to amino acid 164 of HSSTROL3_PEA_{—}1_P5 (SEQ ID NO:365), a second amino acid sequence being at least about 90% or preferably at least about 95% homologous to amino acids 165-358 of MMP11 HUMAN (SEQ ID NO:363), which also corresponds to amino acids 165-358 of HSSTROL3_PEA_{—}1_P5 (SEQ ID NO:365), and a third amino acid sequence being at least 70%, optionally at least 80%, preferably at least 85%, more preferably at least 90% and most preferably at least 95% homologous to a polypeptide having the sequence ELGFPSSTGRDESLEHCRCQGLHK (SEQ ID NO: 653) corresponding to amino acids 359-382 of HSSTROL3_PEA_{—}1_P5 (SEQ ID NO:365), wherein said first amino acid sequence, bridging amino acid, second amino acid sequence and third amino acid sequence are contiguous and in a sequential order.

An isolated polypeptide encoding for a tail of HSSTROL3_PEA_{—}1_P5 (SEQ ID NO:365), comprising a polypeptide being at least 70%, optionally at least about 80%, preferably at least about 85%, more preferably at least about 90% and most preferably at least about 95% homologous to the sequence ELGFPSSTGRDESLEHCRCQGLHK (SEQ ID NO: 653) in HSSTROL3_PEA_{—}1_P5 (SEQ ID NO:365). An isolated chimeric polypeptide encoding for HSSTROL3_PEA_{—}1_P7 (SEQ ID NO:366), comprising a first amino acid sequence being at least about 90% or preferably at least about 95% homologous to amino acids 1-163 of MMP11_HUMAN (SEQ ID NO:363), which also corresponds to amino acids 1-163 of HSSTROL3_PEA_{—}1_P7 (SEQ ID NO:366), a bridging amino acid H corresponding to amino acid 164 of HSSTROL3_PEA_{—}1_P7 (SEQ ID NO:366), a second amino acid sequence being at least about 90% or preferably at least about 95% homologous to amino acids 165-359 of MMP11_HUMAN (SEQ ID NO:363), which also corresponds to amino acids 165-359 of HSSTROL3_PEA_{—}1_P7 (SEQ ID NO:366), and a third amino acid sequence being at least 70%, optionally at least 80%, preferably at least 85%, more preferably at least 90% and most preferably at least 95% homologous to a polypeptide having the sequence TTGVSTPAPGV (SEQ ID NO: 654) corresponding to amino acids 360-370 of HSSTROL3_PEA_{—}1_P7 (SEQ ID NO:366), wherein said first amino acid sequence, bridging amino acid, second amino acid sequence and third amino acid sequence are contiguous and in a sequential order.

An isolated polypeptide encoding for a tail of HSSTROL3_PEA_{—}1_P7 (SEQ ID NO:366), comprising a polypeptide being at least 70%, optionally at least about 80%, preferably at least about 85%, more preferably at least about 90% and most preferably at least about 95% homologous to the sequence TTGVSTPAPGV (SEQ ID NO: 654) in HSSTROL3_PEA_{—}1_P7 (SEQ ID NO:366).

An isolated chimeric polypeptide encoding for HSSTROL3_PEA_{—}1_P8 (SEQ ID NO:367), comprising a first amino acid sequence being at least about 90% or preferably at least about 95% homologous to amino acids 1-163 of MMP11_HUMAN (SEQ ID NO:363), which also corresponds to amino acids 1-163 of HSSTROL3_PEA_{—}1_P8 (SEQ ID NO:367), a bridging amino acid H corresponding to amino acid 164 of HSSTROL3_PEA_{—}1_P8 (SEQ ID NO:367), a second amino acid sequence being at least about 90% or preferably at least about 95% homologous to amino acids 165-286 of MMP11_HUMAN (SEQ ID NO:363), which also corresponds to amino acids 165-286 of HSSTROL3_PEA_{—}1_P8 (SEQ ID NO:367), and a third amino acid sequence being at least 70%, optionally at least 80%, preferably at least 85%, more preferably at least 90% and most preferably at least 95% homologous to a polypeptide having the sequence VRPCLPVPLLLCWPL (SEQ ID NO: 655) corresponding to amino acids 287-301 of HSSTROL3_PEA_{—}1_P8 (SEQ ID NO:367), wherein said first amino acid sequence, bridging amino acid, second amino acid sequence and third amino acid sequence are contiguous and in a sequential order.

An isolated polypeptide encoding for a tail of HSSTROL3_PEA_{—}1_P8 (SEQ ID NO:367), comprising a polypeptide being at least 70%, optionally at least about 80%, preferably at least about 85%, more preferably at least about 90% and most preferably at least about 95% homologous to the sequence VRPCLPVPLLLCWPL (SEQ ID NO: 655) in HSSTROL3_PEA_{—}1_P8 (SEQ ID NO:367).

An isolated chimeric polypeptide encoding for HSSTROL3_PEA_{—}1_P9 (SEQ ID NO:368), comprising a first amino acid sequence being at least about 90% or preferably at least about 95% homologous to amino acids 1-96 of MMP11_HUMAN (SEQ ID NO:363), which also corresponds to amino acids 1-96 of HSSTROL3_PEA_{—}1_P9 (SEQ ID NO:368), a second amino acid sequence being at least about 90% or preferably at least about 95% homologous to amino acids 113-163 of MMP11_HUMAN (SEQ ID NO:363), which also corresponds to amino acids 97-147 of HSSTROL3_PEA_{—}1_P9 (SEQ ID NO:368), a bridging amino acid H corresponding to amino acid 148 of HSSTROL3_PEA_{—}1_P9 (SEQ ID NO:368), a third amino acid sequence being at least about 90% or preferably at least about 95% homologous to amino acids 165-359 of MMP11_HUMAN (SEQ ID NO:363), which also corresponds to amino acids 149-343 of HSSTROL3_PEA_{—}1_P9 (SEQ ID NO:368), and a fourth amino acid sequence being at least 70%, optionally at least 80%, preferably at least 85%, more preferably at least 90% and most preferably at least 95% homologous to a polypeptide having the sequence TTGVSTPAPGV (SEQ ID NO: 654) corresponding to amino acids 344-354 of HSSTROL3_PEA_{—}1_P9 (SEQ ID NO:368), wherein said first amino acid sequence, second amino acid sequence, bridging amino acid, third amino acid sequence and fourth amino acid sequence are contiguous and in a sequential order.

An isolated chimeric polypeptide encoding for an edge portion of HSSTROL3_PEA_{—}1_P9 (SEQ ID NO:368), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KR, having a structure as follows: a sequence starting from any of amino acid numbers 96−x to 96; and ending at any of amino acid numbers 97+((n−2)−x), in which x varies from 0 to n−2.

An isolated polypeptide encoding for a tail of HSSTROL3_PEA_{—}1_P9 (SEQ ID NO:368), comprising a polypeptide being at least 70%, optionally at least about 80%, preferably at least about 85%, more preferably at least about 90% and most preferably at least about 95% homologous to the sequence TTGVSTPAPGV (SEQ ID NO: 654) in HSSTROL3_PEA_{—}1_P9 (SEQ ID NO:368).

An isolated chimeric polypeptide encoding for HSSTROL3_PEA_{—}1_P11 (SEQ ID NO:369), comprising a first amino acid sequence being at least about 90% or preferably at least about 95% homologous to amino acids 1-113 of MMP11_HUMAN (SEQ ID NO:363), which also corresponds to amino acids 1-113 of HSSTROL3_PEA_{—}1_P11 (SEQ ID NO:369).

An isolated chimeric polypeptide encoding for HUMGRP5E_P2 (SEQ ID NO:401), comprising a first amino acid sequence being at least about 90% or preferably at least about 95% homologous to amino acids 1-121 of GRP_HUMAN (SEQ ID NO:400), which also corresponds to amino acids 1-121 of HUMGRP5E_P2 (SEQ ID NO:401), and a second amino acid sequence being at least 70%, optionally at least 80%, preferably at least 85%, more preferably at least 90% and most preferably at least 95% homologous to a polypeptide having the sequence LVDSLLQVLNVKEGTPS (SEQ ID NO: 657) corresponding to amino acids 122-138 of HUMGRP5E_P2 (SEQ ID NO:401), wherein said first amino acid sequence and second amino acid sequence are contiguous and in a sequential order.

An isolated polypeptide encoding for a tail of HUMGRP5E_P2 (SEQ ID NO:401), comprising a polypeptide being at least 70%, optionally at least about 80%, preferably at least about 85%, more preferably at least about 90% and most preferably at least about 95% homologous to the sequence LVDSLLQVLNVKEGTPS (SEQ ID NO: 657) in HUMGRP5E_P2 (SEQ ID NO:401).

An isolated chimeric polypeptide encoding for HUMGRP5E_P3 (SEQ ID NO:402), comprising a first amino acid sequence being at least about 90% or preferably at least about 95% homologous to amino acids 1-121 of GRP_HUMAN (SEQ ID NO:400), which also corresponds to amino acids 1-121 of HUMGRP5E_P3 (SEQ ID NO:402), and a second amino acid sequence being at least 70%, optionally at least 80%, preferably at least 85%, more preferably at least 90% and most preferably at least 95% homologous to a polypeptide having the sequence TLCSRFST (SEQ ID NO: 658) corresponding to amino acids 122-129 of HUMGRP5E_P3 (SEQ ID NO:402), wherein said first amino acid sequence and second amino acid sequence are contiguous and in a sequential order.

An isolated polypeptide encoding for a tail of HUMGRP5E_P3 (SEQ ID NO:402), comprising a polypeptide being at least 70%, optionally at least about 80%, preferably at least about 85%, more preferably at least about 90% and most preferably at least about 95% homologous to the sequence TLCSRFST (SEQ ID NO: 658) in HUMGRP5E_P3 (SEQ ID NO:402).

An isolated chimeric polypeptide encoding for HUMGRP5E_P4 (SEQ ID NO:403), comprising a first amino acid sequence being at least about 90% or preferably at least about 95% homologous to amino acids 1-127 of GRP_HUMAN (SEQ ID NO:400), which also corresponds to amino acids 1-127 of HUMGRP5E_P4 (SEQ ID NO:403), and a second amino acid sequence being at least about 90% or preferably at least about 95% homologous to amino acids 135-148 of GRP_HUMAN (SEQ ID NO:400), which also corresponds to amino acids 128-141 of HUMGRP5E_P4 (SEQ ID NO:403), wherein said first amino acid sequence and second amino acid sequence are contiguous and in a sequential order.

An isolated chimeric polypeptide encoding for an edge portion of HUMGRP5E_P4 (SEQ ID NO:403), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KG, having a structure as follows: a sequence starting from any of amino acid numbers 127−x to 127; and ending at any of amino acid numbers 128+((n−2)−x), in which x varies from 0 to n−2.

An isolated chimeric polypeptide encoding for HUMGRP5E_P5 (SEQ ID NO:404), comprising a first amino acid sequence being at least about 90% or preferably at least about 95% homologous to amino acids 1-127 of GRP_HUMAN (SEQ ID NO:400), which also corresponds to amino acids 1-127 of HUMGRP5E_P5 (SEQ ID NO:404), and a second amino acid sequence being at least 70%, optionally at least 80%, preferably at least 85%, more preferably at least 90% and most preferably at least 95% homologous to a polypeptide having the sequence DSLLQVLNVKEGTPS (SEQ ID NO: 659) corresponding to amino acids 128-142 of HUMGRP5E_P5 (SEQ ID NO:404), wherein said first amino acid sequence and second amino acid sequence are contiguous and in a sequential order.

An isolated polypeptide encoding for a tail of HUMGRP5E_P5 (SEQ ID NO:404), comprising a polypeptide being at least 70%, optionally at least about 80%, preferably at least about 85%, more preferably at least about 90% and most preferably at least about 95% homologous to the sequence DSLLQVLNVKEGTPS (SEQ ID NO: 659) in HUMGRP5E_P5 (SEQ ID NO:404).

An isolated chimeric polypeptide encoding for T94936_PEA_{—}1_PEA_{—}1_P2 (SEQ ID NO:427), comprising a first amino acid sequence being at least about 90% or preferably at least about 95% homologous to amino acids 1-150 of Q8TD06 (SEQ ID NO:695) (SEQ ID NO:426), which also corresponds to amino acids 1-150 of T94936_PEA_{—}1_PEA_{—}1_P2 (SEQ ID NO:427).

An isolated chimeric polypeptide encoding for T94936_PEA_{—}1_PEA_{—}1_P3 (SEQ ID NO:428), comprising a first amino acid sequence being at least about 90% or preferably at least about 95% homologous to amino acids 1-122 of Q8TD06 (SEQ ID NO:695) (SEQ ID NO:426), which also corresponds to amino acids 1-122 of T94936_PEA_{—}1_PEA_{—}1_P3 (SEQ ID NO:428), and a second amino acid sequence being at least 70%, optionally at least 80%, preferably at least 85%, more preferably at least 90% and most preferably at least 95% homologous to a polypeptide having the sequence GMYVISFHQIYKISRNQHSCFYF (SEQ ID NO: 660) corresponding to amino acids 123-145 of T94936_PEA_{—}1_PEA_{—}1_P3 (SEQ ID NO:428), wherein said first amino acid sequence and second amino acid sequence are contiguous and in a sequential order.

An isolated polypeptide encoding for a tail of T94936_PEA_{—}1_PEA_{—}1_P3 (SEQ ID NO:428), comprising a polypeptide being at least 70%, optionally at least about 80%, preferably at least about 85%, more preferably at least about 90% and most preferably at least about 95% homologous to the sequence GMYVISFHQIYKISRNQHSCFYF (SEQ ID NO: 660) in T94936_PEA_{—}1_PEA_{—}1_P3 (SEQ ID NO:428).

An isolated chimeric polypeptide encoding for T94936_PEA_{—}1_PEA_{—}1_P7 (SEQ ID NO:429), comprising a first amino acid sequence being at least about 90% or preferably at least about 95% homologous to amino acids 1-57 of Q8TD06 (SEQ ID NO:695) (SEQ ID NO:426), which also corresponds to amino acids 1-57 of T94936_PEA_{—}1_PEA_{—}1_P7 (SEQ ID NO:429), and a second amino acid sequence being at least 70%, optionally at least 80%, preferably at least 85%, more preferably at least 90% and most preferably at least 95% homologous to a polypeptide having the sequence RSH corresponding to amino acids 58-60 of T94936_PEA_{—}1_PEA_{—}1_P7 (SEQ ID NO:429), wherein said first amino acid sequence and second amino acid sequence are contiguous and in a sequential order.

An isolated chimeric polypeptide encoding for HSTGFB1_P2 (SEQ ID NO:464), comprising a first amino acid sequence being at least about 90% or preferably at least about 95% homologous to amino acids 1-238 of TGFB1_HUMAN (SEQ ID NO:463), which also corresponds to amino acids 1-238 of HSTGFB1_P2 (SEQ ID NO:464), and a second amino acid sequence being at least 70%, optionally at least 80%, preferably at least 85%, more preferably at least 90% and most preferably at least 95% homologous to a polypeptide having the sequence EACFPGHAQL (SEQ ID NO: 661) corresponding to amino acids 239-248 of HSTGFB1_P2 (SEQ ID NO:464), wherein said first amino acid sequence and second amino acid sequence are contiguous and in a sequential order.

An isolated polypeptide encoding for a tail of HSTGFB1_P2 (SEQ ID NO:464), comprising a polypeptide being at least 70%, optionally at least about 80%, preferably at least about 85%, more preferably at least about 90% and most preferably at least about 95% homologous to the sequence EACFPGHAQL (SEQ ID NO: 661) in HSTGFB1_P2 (SEQ ID NO:464).

An isolated chimeric polypeptide encoding for HSTGFB1_P3 (SEQ ID NO:465), comprising a first amino acid sequence being at least about 90% or preferably at least about 95% homologous to amino acids 1-339 of TGFB1_HUMAN (SEQ ID NO:463), which also corresponds to amino acids 1-339 of HSTGFB1_P3 (SEQ ID NO:465), and a second amino acid sequence being at least 70%, optionally at least 80%, preferably at least 85%, more preferably at least 90% and most preferably at least 95% homologous to a polypeptide having the sequence RLAHRATRCAWGEPGRRKRREKEK (SEQ ID NO: 662) corresponding to amino acids 340-363 of HSTGFB1_P3 (SEQ ID NO:465), wherein said first amino acid sequence and second amino acid sequence are contiguous and in a sequential order.

An isolated polypeptide encoding for a tail of HSTGFB1_P3 (SEQ ID NO:465), comprising a polypeptide being at least 70%, optionally at least about 80%, preferably at least about 85%, more preferably at least about 90% and most preferably at least about 95% homologous to the sequence RLAHRATRCAWGEPGRRKRREKEK (SEQ ID NO: 662) in HSTGFB1_P3 (SEQ ID NO:465).

An isolated chimeric polypeptide encoding for HSTGFB1_P5 (SEQ ID NO:466), comprising a first amino acid sequence being at least about 90% or preferably at least about 95% homologous to amino acids 1-338 of TGFB1_HUMAN (SEQ ID NO:463), which also corresponds to amino acids 1-338 of HSTGFB1_P5 (SEQ ID NO:466), and a second amino acid sequence being at least 70%, optionally at least 80%, preferably at least 85%, more preferably at least 90% and most preferably at least 95% homologous to a polypeptide having the sequence LNEQNLIQEVPNIWQREVG (SEQ ID NO: 663) corresponding to amino acids 339-357 of HSTGFB1_P5 (SEQ ID NO:466), wherein said first amino acid sequence and second amino acid sequence are contiguous and in a sequential order.

An isolated polypeptide encoding for a tail of HSTGFB1_P5 (SEQ ID NO:466), comprising a polypeptide being at least 70%, optionally at least about 80%, preferably at least about 85%, more preferably at least about 90% and most preferably at least about 95% homologous to the sequence LNEQNLIQEVPNIWQREVG (SEQ ID NO: 663) in HSTGFB1_P5 (SEQ ID NO:466).

An isolated chimeric polypeptide encoding for HSTGFB1_P7 (SEQ ID NO:467), comprising a first amino acid sequence being at least about 90% or preferably at least about 95% homologous to amino acids 1-237 of TGFB1_HUMAN (SEQ ID NO:463), which also corresponds to amino acids 1-237 of HSTGFB1_P7 (SEQ ID NO:467), and a second amino acid sequence being at least 70%, optionally at least 80%, preferably at least 85%, more preferably at least 90% and most preferably at least 95% homologous to a polypeptide having the sequence APRRRTAACGSCTLTSARTSAGSGSTSPRATMPTSASGPAPTFGAWTRSTARSWPCTTSITRAPRRR RAACRRRWSRCPSCTTWAASPRWASSCPT (SEQ ID NO: 664) corresponding to amino acids 238-332 of HSTGFB1_P7 (SEQ ID NO:467), wherein said first amino acid sequence and second amino acid sequence are contiguous and in a sequential order.

An isolated polypeptide encoding for a tail of HSTGFB1_P7 (SEQ ID NO:467), comprising a polypeptide being at least 70%, optionally at least about 80%, preferably at least about 85%, more preferably at least about 90% and most preferably at least about 95% homologous to the sequence APRRRTAACGSCTLTSARTSAGSGSTSPRATMPTSASGPAPTFGAWTRSTARSWPCTTSITRAPRRR RAACRRRWSRCPSCTTWAASPRWASSCPT (SEQ ID NO: 664) in HSTGFB1_P7 (SEQ ID NO:467).

An isolated chimeric polypeptide encoding for Z36249_PEA_{—}3_P2 (SEQ ID NO:579), comprising a first amino acid sequence being at least about 90% or preferably at least about 95% homologous to amino acids 1-115 of Q96LE7, which also corresponds to amino acids 1-115 of Z36249_PEA_{—}3_P2 (SEQ ID NO:579), and a second amino acid sequence being at least about 90% or preferably at least about 95% homologous to amino acids 152-319 of Q96LE7, which also corresponds to amino acids 116-283 of Z36249_PEA_{—}3_P2 (SEQ ID NO:579), wherein said first amino acid sequence and second amino acid sequence are contiguous and in a sequential order.

An isolated chimeric polypeptide encoding for an edge portion of Z36249_PEA_{—}3_P2 (SEQ ID NO:579), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IY, having a structure as follows: a sequence starting from any of amino acid numbers 115−x to 115; and ending at any of amino acid numbers 116+((n−2)−x), in which x varies from 0 to n−2.

An isolated chimeric polypeptide encoding for Z36249_PEA_{—}3_P2 (SEQ ID NO:579), comprising a first amino acid sequence being at least about 90% or preferably at least about 95% homologous to amino acids 1-70 of Q15327, which also corresponds to amino acids 1-70 of Z36249_PEA_{—}3_P2 (SEQ ID NO:579), a bridging amino acid K corresponding to amino acid 71 of Z36249_PEA_{—}3_P2 (SEQ ID NO:579), a second amino acid sequence being at least about 90% or preferably at least about 95% homologous to amino acids 72-115 of Q15327, which also corresponds to amino acids 72-115 of Z36249_PEA_{—}3_P2 (SEQ ID NO:579), and a third amino acid sequence being at least about 90% or preferably at least about 95% homologous to amino acids 152-319 of Q15327, which also corresponds to amino acids 116-283 of Z36249_PEA_{—}3_P2 (SEQ ID NO:579), wherein said first amino acid sequence, bridging amino acid, second amino acid sequence and third amino acid sequence are contiguous and in a sequential order.

An isolated chimeric polypeptide encoding for an edge portion of Z36249_PEA_{—}3_P2 (SEQ ID NO:579), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IY, having a structure as follows: a sequence starting from any of amino acid numbers 115−x to 115; and ending at any of amino acid numbers 116+((n−2)−x), in which x varies from 0 to n−2.

An isolated chimeric polypeptide encoding for Z36249_PEA_{—}3_P3 (SEQ ID NO:580), comprising a first amino acid sequence being at least about 90% or preferably at least about 95% homologous to amino acids 1-184 of Q96LE7, which also corresponds to amino acids 1-184 of Z36249_PEA_{—}3_P3 (SEQ ID NO:580), and a second amino acid sequence being at least 70%, optionally at least 80%, preferably at least 85%, more preferably at least 90% and most preferably at least 95% homologous to a polypeptide having the sequence VNIFLCLGMSQKK (SEQ ID NO: 665) corresponding to amino acids 185-197 of Z36249_PEA_{—}3_P3 (SEQ ID NO:580), wherein said first amino acid sequence and second amino acid sequence are contiguous and in a sequential order.

An isolated polypeptide encoding for a tail of Z36249_PEA_{—}3_P3 (SEQ ID NO:580), comprising a polypeptide being at least 70%, optionally at least about 80%, preferably at least about 85%, more preferably at least about 90% and most preferably at least about 95% homologous to the sequence VNIFLCLGMSQKK (SEQ ID NO: 665) in Z36249_PEA_{—}3_P3 (SEQ ID NO:580).

An isolated chimeric polypeptide encoding for Z36249_PEA_{—}3_P3 (SEQ ID NO:580), comprising a first amino acid sequence being at least about 90% or preferably at least about 95% homologous to amino acids 1-70 of Q15327, which also corresponds to amino acids 1-70 of Z36249_PEA_{—}3_P3 (SEQ ID NO:580), a bridging amino acid K corresponding to amino acid 71 of Z36249_PEA_{—}3_P3 (SEQ ID NO:580), a second amino acid sequence being at least about 90% or preferably at least about 95% homologous to amino acids 72-184 of Q15327, which also corresponds to amino acids 72-184 of Z36249_PEA_{—}3_P3 (SEQ ID NO:580), and a third amino acid sequence being at least 70%, optionally at least 80%, preferably at least 85%, more preferably at least 90% and most preferably at least 95% homologous to a polypeptide having the sequence VNIFLCLGMSQKK (SEQ ID NO: 665) corresponding to amino acids 185-197 of Z36249_PEA_{—}3_P3 (SEQ ID NO:580), wherein said first amino acid sequence, bridging amino acid, second amino acid sequence and third amino acid sequence are contiguous and in a sequential order.

An isolated polypeptide encoding for a tail of Z36249_PEA_{—}3_P3 (SEQ ID NO:580), comprising a polypeptide being at least 70%, optionally at least about 80%, preferably at least about 85%, more preferably at least about 90% and most preferably at least about 95% homologous to the sequence VNIFLCLGMSQKK (SEQ ID NO: 665) in Z36249_PEA_{—}3_P3 (SEQ ID NO:580).

An isolated chimeric polypeptide encoding for Z36249_PEA_{—}3_P4 (SEQ ID NO:581), comprising a first amino acid sequence being at least about 90% or preferably at least about 95% homologous to amino acids 1-151 of Q96LE7, which also corresponds to amino acids 1-151 of Z36249_PEA_{—}3_P4 (SEQ ID NO:581), and a second amino acid sequence being at least 70%, optionally at least 80%, preferably at least 85%, more preferably at least 90% and most preferably at least 95% homologous to a polypeptide having the sequence VRLMQSTAKSSSLILCFLCFTPVLLI (SEQ ID NO: 666) corresponding to amino acids 152-177 of Z36249_PEA_{—}3_P4 (SEQ ID NO:581), wherein said first amino acid sequence and second amino acid sequence are contiguous and in a sequential order.

An isolated polypeptide encoding for a tail of Z36249_PEA_{—}3_P4 (SEQ ID NO:581), comprising a polypeptide being at least 70%, optionally at least about 80%, preferably at least about 85%, more preferably at least about 90% and most preferably at least about 95% homologous to the sequence VRLMQSTAKSSSLILCFLCFTPVLLI (SEQ ID NO: 666) in Z36249_PEA_{—}3_P4 (SEQ ID NO:581).

An isolated chimeric polypeptide encoding for Z36249_PEA_{—}3_P4 (SEQ ID NO:581), comprising a first amino acid sequence being at least about 90% or preferably at least about 95% homologous to amino acids 1-70 of Q15327, which also corresponds to amino acids 1-70 of Z36249_PEA_{—}3_P4 (SEQ ID NO:581), a bridging amino acid K corresponding to amino acid 71 of Z36249_PEA_{—}3_P4 (SEQ ID NO:581), a second amino acid sequence being at least about 90% or preferably at least about 95% homologous to amino acids 72-151 of Q15327, which also corresponds to amino acids 72-151 of Z36249_PEA_{—}3_P4 (SEQ ID NO: 581), and a third amino acid sequence being at least 70%, optionally at least 80%, preferably at least 85%, more preferably at least 90% and most preferably at least 95% homologous to a polypeptide having the sequence VRLMQSTAKSSSLILCFLCFTPVLLI (SEQ ID NO: 666) corresponding to amino acids 152-177 of Z36249_PEA_{—}3_P4 (SEQ ID NO:581), wherein said first amino acid sequence, bridging amino acid, second amino acid sequence and third amino acid sequence are contiguous and in a sequential order.

An isolated polypeptide encoding for a tail of Z36249_PEA_{—}3_P4 (SEQ ID NO: 581), comprising a polypeptide being at least 70%, optionally at least about 80%, preferably at least about 85%, more preferably at least about 90% and most preferably at least about 95% homologous to the sequence VRLMQSTAKSSSLILCFLCFTPVLLI (SEQ ID NO: 666) in Z36249_PEA_{—}3_P4 (SEQ ID NO:581).

An isolated chimeric polypeptide encoding for Z36249_PEA_{—}3_P5 (SEQ ID NO:582), comprising a first amino acid sequence being at least about 90% or preferably at least about 95% homologous to amino acids 1-151 of Q96LE7, which also corresponds to amino acids 1-151 of Z36249_PEA_{—}3_P5 (SEQ ID NO:582), and a second amino acid sequence being at least about 90% or preferably at least about 95% homologous to amino acids 185-319 of Q96LE7, which also corresponds to amino acids 152-286 of Z36249_PEA_{—}3_P5 (SEQ ID NO:582), wherein said first amino acid sequence and second amino acid sequence are contiguous and in a sequential order.

An isolated chimeric polypeptide encoding for an edge portion of Z36249_PEA_{—}3_P5 (SEQ ID NO:582), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EL, having a structure as follows: a sequence starting from any of amino acid numbers 151−x to 151; and ending at any of amino acid numbers 152+((n−2)−x), in which x varies from 0 to n−2.

An isolated chimeric polypeptide encoding for Z36249_PEA_{—}3_P5 (SEQ ID NO:582), comprising a first amino acid sequence being at least about 90% or preferably at least about 95% homologous to amino acids 1-70 of Q15327, which also corresponds to amino acids 1-70 of Z36249_PEA_{—}3_P5 (SEQ ID NO:582), a bridging amino acid K corresponding to amino acid 71 of Z36249_PEA_{—}3_P5 (SEQ ID NO:582), a second amino acid sequence being at least about 90% or preferably at least about 95% homologous to amino acids 72-151 of Q15327, which also corresponds to amino acids 72-151 of Z36249_PEA_{—}3_P5 (SEQ ID NO:582), and a third amino acid sequence being at least about 90% or preferably at least about 95% homologous to amino acids 185-319 of Q15327, which also corresponds to amino acids 152-286 of Z36249_PEA_{—}3_P5 (SEQ ID NO:582), wherein said first amino acid sequence, bridging amino acid, second amino acid sequence and third amino acid sequence are contiguous and in a sequential order.

An isolated chimeric polypeptide encoding for an edge portion of Z36249_PEA_{—}3_P5 (SEQ ID NO:582), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EL, having a structure as follows: a sequence starting from any of amino acid numbers 151−x to 151; and ending at any of amino acid numbers 152+((n−2)−x), in which x varies from 0 to n−2.

An isolated chimeric polypeptide encoding for M78530_PEA_{—}1_P15 (SEQ ID NO:619), comprising a first amino acid sequence being at least about 90% or preferably at least about 95% homologous to amino acids 1-544 of Q9HCB6 (SEQ ID NO:617), which also corresponds to amino acids 1-544 of M78530_PEA_{—}1_P15 (SEQ ID NO:619), a bridging amino acid T corresponding to amino acid 545 of M78530_PEA_{—}1_P15 (SEQ ID NO:619), a second amino acid sequence being at least about 90% or preferably at least about 95% homologous to amino acids 546-665 of Q9HCB6 (SEQ ID NO:617), which also corresponds to amino acids 546-665 of M78530_PEA_{—}1_P15 (SEQ ID NO:619), and a third amino acid sequence being at least 70%, optionally at least 80%, preferably at least 85%, more preferably at least 90% and most preferably at least 95% homologous to a polypeptide having the sequence RKSWSSSRPITSMFLSPGSPEPASANTARS (SEQ ID NO: 667) corresponding to amino acids 666-695 of M78530_PEA_{—}1_P15 (SEQ ID NO:619), wherein said first amino acid sequence, bridging amino acid, second amino acid sequence and third amino acid sequence are contiguous and in a sequential order.

An isolated polypeptide encoding for a tail of M78530_PEA_{—}1_P15 (SEQ ID NO:619), comprising a polypeptide being at least 70%, optionally at least about 80%, preferably at least about 85%, more preferably at least about 90% and most preferably at least about 95% homologous to the sequence RKSWSSSRPITSMFLSPGSPEPASANTARS (SEQ ID NO: 667) in M78530_PEA_{—}1_P15 (SEQ ID NO:619).

An isolated chimeric polypeptide encoding for M78530_PEA_{—}1_P15 (SEQ ID NO:619), comprising a first amino acid sequence being at least 70%, optionally at least 80%, preferably at least 85%, more preferably at least 90% and most preferably at least 95% homologous to a polypeptide having the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) corresponding to amino acids 1-83 of M78530_PEA_{—}1_P15 (SEQ ID NO:619), a second amino acid sequence being at least about 90% or preferably at least about 95% homologous to amino acids 1-582 of O94862 (SEQ ID NO:618), which also corresponds to amino acids 84-665 of M78530_PEA_{—}1_P15 (SEQ ID NO:619), and a third amino acid sequence being at least 70%, optionally at least 80%, preferably at least 85%, more preferably at least 90% and most preferably at least 95% homologous to a polypeptide having the sequence RKSWSSSRPITSMFLSPGSPEPASANTARS (SEQ ID NO: 667) corresponding to amino acids 666-695 of M78530_PEA_{—}1_P15 (SEQ ID NO:619), wherein said first amino acid sequence, second amino acid sequence and third amino acid sequence are contiguous and in a sequential order.

An isolated polypeptide encoding for a head of M78530_PEA_{—}1_P15 (SEQ ID NO:619), comprising a polypeptide being at least 70%, optionally at least about 80%, preferably at least about 85%, more preferably at least about 90% and most preferably at least about 95% homologous to the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) of M78530_PEA_{—}1_P15 (SEQ ID NO:619).

An isolated polypeptide encoding for a tail of M78530_PEA_{—}1_P15 (SEQ ID NO:619), comprising a polypeptide being at least 70%, optionally at least about 80%, preferably at least about 85%, more preferably at least about 90% and most preferably at least about 95% homologous to the sequence RKSWSSSRPITSMFLSPGSPEPASANTARS (SEQ ID NO: 667) in M78530_PEA_{—}1_P15 (SEQ ID NO:619).

An isolated chimeric polypeptide encoding for M78530_PEA_{—}1_P16 (SEQ ID NO:620), comprising a first amino acid sequence being at least about 90% or preferably at least about 95% homologous to amino acids 1-297 of Q8NCD7 (SEQ ID NO:616), which also corresponds to amino acids 1-297 of M78530_PEA_{—}1_P16 (SEQ ID NO:620).

An isolated chimeric polypeptide encoding for M78530_PEA_{—}1_P16 (SEQ ID NO:620), comprising a first amino acid sequence being at least about 90% or preferably at least about 95% homologous to amino acids 1-297 of Q9HCB6 (SEQ ID NO:617), which also corresponds to amino acids 1-297 of M78530_PEA_{—}1_P16 (SEQ ID NO:620).

An isolated chimeric polypeptide encoding for M78530_PEA_{—}1_P16 (SEQ ID NO:620), comprising a first amino acid sequence being at least 70%, optionally at least 80%, preferably at least 85%, more preferably at least 90% and most preferably at least 95% homologous to a polypeptide having the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) corresponding to amino acids 1-83 of M78530_PEA_{—}1_P16 (SEQ ID NO:620), and a second amino acid sequence being at least about 90% or preferably at least about 95% homologous to amino acids 1-214 of O94862 (SEQ ID NO:618), which also corresponds to amino acids 84-297 of M78530_PEA_{—}1_P16 (SEQ ID NO:620), wherein said first amino acid sequence and second amino acid sequence are contiguous and in a sequential order.

An isolated polypeptide encoding for a head of M78530_PEA_{—}1_P16 (SEQ ID NO:620), comprising a polypeptide being at least 70%, optionally at least about 80%, preferably at least about 85%, more preferably at least about 90% and most preferably at least about 95% homologous to the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) of M78530_PEA_{—}1_P16 (SEQ ID NO:620).

An isolated chimeric polypeptide encoding for M78530_PEA_{—}1_P17 (SEQ ID NO:621), comprising a first amino acid sequence being at least about 90% or preferably at least about 95% homologous to amino acids 1-275 of Q8NCD7 (SEQ ID NO:616), which also corresponds to amino acids 1-275 of M78530_PEA_{—}1_P17 (SEQ ID NO:621), and a second amino acid sequence being at least 70%, optionally at least 80%, preferably at least 85%, more preferably at least 90% and most preferably at least 95% homologous to a polypeptide having the sequence VRQKNHRMTK (SEQ ID NO: 670) corresponding to amino acids 276-285 of M78530_PEA_{—}1_P17 (SEQ ID NO:621), wherein said first amino acid sequence and second amino acid sequence are contiguous and in a sequential order.

An isolated polypeptide encoding for a tail of M78530_PEA_{—}1_P17 (SEQ ID NO:621), comprising a polypeptide being at least 70%, optionally at least about 80%, preferably at least about 85%, more preferably at least about 90% and most preferably at least about 95% homologous to the sequence VRQKNHRMTK (SEQ ID NO: 670) in M78530_PEA_{—}1_P17 (SEQ ID NO:621).

An isolated chimeric polypeptide encoding for M78530_PEA_{—}1_P17 (SEQ ID NO:621), comprising a first amino acid sequence being at least about 90% or preferably at least about 95% homologous to amino acids 1-275 of Q9HCB6 (SEQ ID NO:617), which also corresponds to amino acids 1-275 of M78530_PEA_{—}1_P17 (SEQ ID NO:621), and a second amino acid sequence being at least 70%, optionally at least 80%, preferably at least 85%, more preferably at least 90% and most preferably at least 95% homologous to a polypeptide having the sequence VRQKNHRMTK (SEQ ID NO: 670) corresponding to amino acids 276-285 of M78530 PEA_{—}1_P17 (SEQ ID NO:621), wherein said first amino acid sequence and second amino acid sequence are contiguous and in a sequential order.

An isolated polypeptide encoding for a tail of M78530_PEA_{—}1_P17 (SEQ ID NO:621), comprising a polypeptide being at least 70%, optionally at least about 80%, preferably at least about 85%, more preferably at least about 90% and most preferably at least about 95% homologous to the sequence VRQKNHRMTK (SEQ ID NO: 670) in M78530_PEA_{—}1_P17 (SEQ ID NO:621).

An isolated chimeric polypeptide encoding for M78530_PEA_{—}1_P17 (SEQ ID NO:621), comprising a first amino acid sequence being at least 70%, optionally at least 80%, preferably at least 85%, more preferably at least 90% and most preferably at least 95% homologous to a polypeptide having the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) corresponding to amino acids 1-83 of M78530_PEA_{—}1_P17 (SEQ ID NO:621), a second amino acid sequence being at least about 90% or preferably at least about 95% homologous to amino acids 1-192 of O94862 (SEQ ID NO:618), which also corresponds to amino acids 84-275 of M78530_PEA_{—}1_P17 (SEQ ID NO:621), and a third amino acid sequence being at least 70%, optionally at least 80%, preferably at least 85%, more preferably at least 90% and most preferably at least 95% homologous to a polypeptide having the sequence VRQKNHRMTK (SEQ ID NO: 670) corresponding to amino acids 276-285 of M78530_PEA_{—}1_P17 (SEQ ID NO:621), wherein said first amino acid sequence, second amino acid sequence and third amino acid sequence are contiguous and in a sequential order.

An isolated polypeptide encoding for a head of M78530_PEA_{—}1_P17 (SEQ ID NO:621), comprising a polypeptide being at least 70%, optionally at least about 80%, preferably at least about 85%, more preferably at least about 90% and most preferably at least about 95% homologous to the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) of M78530_PEA_{—}1_P17 (SEQ ID NO:621).

An isolated polypeptide encoding for a tail of M78530_PEA_{—}1_P17 (SEQ ID NO:621), comprising a polypeptide being at least 70%, optionally at least about 80%, preferably at least about 85%, more preferably at least about 90% and most preferably at least about 95% homologous to the sequence VRQKNHRMTK (SEQ ID NO: 670) in M78530_PEA_{—}1_P17 (SEQ ID NO:621).

An isolated chimeric polypeptide encoding for S57296_{—}1_P59 (SEQ ID NO:542), comprising a first amino acid sequence being at least 90% homologous or preferably at least about 95% to amino acids 1-383 of ERB2_HUMAN (SEQ ID NO:538), which also corresponds to amino acids 1-383 of S57296_{—}1_P59 (SEQ ID NO:542), a second amino acid sequence being at least 70%, optionally at least 80%, preferably at least 85%, more preferably at least 90% and most preferably at least 95% homologous to a polypeptide having the sequence VSLCQQAGVQWYDLGSLQPLPPGFKQFSCLSLLSSWDYR (SEQ ID NO: 672) corresponding to amino acids 384-422 of S57296_{—}1_P59 (SEQ ID NO:542), and a third amino acid sequence being at least 90% or preferably at least about 95% homologous to amino acids 384-1255 of ERB2_HUMAN (SEQ ID NO:538), which also corresponds to amino acids 423-1294 of S57296_{—}1_P59 (SEQ ID NO:542), wherein said first amino acid sequence, second amino acid sequence and third amino acid sequence are contiguous and in a sequential order.

An isolated polypeptide encoding for an edge portion of S57296_{—}1_P59 (SEQ ID NO:542), comprising an amino acid sequence being at least 70%, optionally at least about 80%, preferably at least about 85%, more preferably at least about 90% and most preferably at least about 95% homologous to the sequence VSLCQQAGVQWYDLGSLQPLPPGFKQFSCLSLLSSWDYR (SEQ ID NO: 672) of S57296_{—}1_P59 (SEQ ID NO:542).

An isolated chimeric polypeptide encoding for S57296_{—}1_P59 (SEQ ID NO:542), comprising a first amino acid sequence being at least 90% or preferably at least about 95% homologous to amino acids 1-383 of NP_{—}004439 (SEQ ID NO:540), which also corresponds to amino acids 1-383 of S57296_{—}1_P59 (SEQ ID NO:542), a second amino acid sequence being at least 70%, optionally at least 80%, preferably at least 85%, more preferably at least 90% and most preferably at least 95% homologous to a polypeptide having the sequence VSLCQQAGVQWYDLGSLQPLPPGFKQFSCLSLLSSWDYR (SEQ ID NO: 672) corresponding to amino acids 384-422 of S57296_{—}1_P59 (SEQ ID NO:542), and a third amino acid sequence being at least 90% or preferably at least about 95% homologous to amino acids 384-1255 of NP_{—}004439 (SEQ ID NO:540), which also corresponds to amino acids 423-1294 of S57296_{—}1_P59 (SEQ ID NO:542), wherein said first amino acid sequence, second amino acid sequence and third amino acid sequence are contiguous and in a sequential order.

An isolated polypeptide encoding for an edge portion of S57296_{—}1_P59 (SEQ ID NO:542), comprising an amino acid sequence being at least 70%, optionally at least about 80%, preferably at least about 85%, more preferably at least about 90% and most preferably at least about 95% homologous to the sequence VSLCQQAGVQWYDLGSLQPLPPGFKQFSCLSLLSSWDYR (SEQ ID NO: 672) of S57296_{—}1_P59 (SEQ ID NO:542).

An isolated chimeric polypeptide encoding for S57296_{—}1_P59 (SEQ ID NO:542), comprising a first amino acid sequence being at least 70%, optionally at least 80%, preferably at least 85%, more preferably at least 90% and most preferably at least 95%, homologous to a polypeptide having the sequence MELAALCRWGLLLALLPPGAASTQVCTGTD (SEQ ID NO: 673) corresponding to amino acids 1-30 of S57296_{—}1_P59 (SEQ ID NO:542), a second amino acid sequence being at least 70%, optionally at least 80%, preferably at least 85%, more preferably at least 90% and most preferably at least 95% homologous to a polypeptide having the sequence MKLRLPASPETHLDMLRHLYQGCQVVQGNLELTYLPTNA (SEQ ID NO: 674) corresponding to amino acids 384-422 of S57296_{—}1_P59 (SEQ ID NO:542), a third amino acid sequence being at least 90% or preferably at least about 95% homologous to amino acids 354-1225 of NP_{—}001005862 (SEQ ID NO:539), which also corresponds to amino acids 423-1294 of S57296_{—}1_P59 (SEQ ID NO:542), and a fourth amino acid sequence being at least 90% or preferably at least about 95% homologous to amino acids 1-353 of NP_{—}001005862 (SEQ ID NO:539), which also corresponds to amino acids 31-383 of S57296_{—}1_P59 (SEQ ID NO:542), wherein said first amino acid sequence, second amino acid sequence, third amino acid sequence and fourth amino acid sequence are contiguous and in a sequential order.

An isolated polypeptide encoding for a head of S57296_{—}1_P59 (SEQ ID NO:542), comprising a polypeptide being at least 70%, optionally at least about 80%, preferably at least about 85%, more preferably at least about 90% and most preferably at least about 95% homologous to the sequence MELAALCRWGLLLALLPPGAASTQVCTGTD (SEQ ID NO: 673) of S57296_{—}1_P59 (SEQ ID NO:542).

An isolated polypeptide encoding for an edge portion of S57296_{—}1_P59 (SEQ ID NO:542), comprising an amino acid sequence being at least 70%, optionally at least about 80%, preferably at least about 85%, more preferably at least about 90% and most preferably at least about 95% homologous to the sequence MKLRLPASPETHLDMLRHLYQGCQVVQGNLELTYLPTNA (SEQ ID NO: 674) of S57296_{—}1_P59 (SEQ ID NO:542).

An isolated chimeric polypeptide encoding for S57296_{—}1_P65 (SEQ ID NO:543), comprising a first amino acid sequence being at least 90% or preferably at least about 95% homologous to amino acids 1-340 of Q9UK79_HUMAN (SEQ ID NO:534), which also corresponds to amino acids 1-340 of S57296_{—}1_P65 (SEQ ID NO:543), and a second amino acid sequence being at least 70%, optionally at least 80%, preferably at least 85%, more preferably at least 90% and most preferably at least 95% homologous to a polypeptide having the sequence VCYGLGMEHLREVRAVTSANIQEFAGCKKIFGSLAFLPESFDGDPASNTAPLQPEQLQVFETLEEIT GYLYISAWPDSLPDLSVFQNLQVIRGRILHNGAYSLTLQGLGISWLGLRSLRELGSGLALIHHNTHL CFVHTVPWDQLFRNPHQALLHTANRPEDECGKTGSPVCALPICQHTAVPRGPWQQRSWTCADCP SLCTLLDSAQLWLAWPLGMASLAGSYLPWHPSLPLCF (SEQ ID NO: 675) corresponding to amino acids 341-575 of S57296_{—}1_P65 (SEQ ID NO:543), wherein said first amino acid sequence and second amino acid sequence are contiguous and in a sequential order.

An isolated polypeptide encoding for an edge portion of S57296_{—}1_P65 (SEQ ID NO:543), comprising an amino acid sequence being at least 70%, optionally at least about 80%, preferably at least about 85%, more preferably at least about 90% and most preferably at least about 95% homologous to the sequence

(SEQ ID NO:675) of S57296_1_P65 (SEQ ID NO:543)

VCYGLGMEHLREVRAVTSANIQEFAGCKKIFGSLAFLPESFDGDPASNTA
PLQPEQLQVFETLEEITGYLYISAWPDSLPDLSVFQNLQVIRGRILHNGA
YSLTLQGLGISWLGLRSLRELGSGLALIHHNTHLCFVHTVPWDQLFRNPH
QALLHTANRPEDECGKTGSPVCALPICQHTAVPRGPWQQRSWTCADCPSL
CTLLDSAQLWLAWPLGMASLAGSYLPWHPSLPLCF.

An isolated chimeric polypeptide encoding for S57296_{—}1_P85 (SEQ ID NO:544), comprising a first amino acid sequence being at least 90% or preferably at least about 95% homologous to amino acids 1-340 of Q9UK79_HUMAN (SEQ ID NO:534), which also corresponds to amino acids 1-340 of S57296_{—}1_P85 (SEQ ID NO:544), and a second amino acid sequence being at least 70%, optionally at least 80%, preferably at least 85%, more preferably at least 90% and most preferably at least 95% homologous to a polypeptide having the sequence VCYGLGMEHLREVRAVTSANIQEFAGCKKIFGSLAFLPESFDGVSLCQQAGVQWYDLGSLQPLPP GFKQFSCLSLLSSWDYRDPASNTAPLQPEQLQVFETLEEITGYLYISAWPDSLPDLSVFQNLQVIRG RILHNGAYSLTLQGLGISWLGLRSLRELGSGLALIHHNTHLCFVHTVPWDQLFRNPHQALLHTANR PEDECGKTGSPVCALPICQHTAVPRGPWQQRSWTCADCPSLCTLLDSAQLWLAWPLGMASLAGS YLPWHPSLPLCF (SEQ ID NO: 676) corresponding to amino acids 341-614 of S57296_{—}1_P85 (SEQ ID NO:544), wherein said first amino acid sequence and second amino acid sequence are contiguous and in a sequential order.

An isolated polypeptide encoding for an edge portion of S57296_{—}1_P85 (SEQ ID NO:544), comprising an amino acid sequence being at least 70%, optionally at least about 80%, preferably at least about 85%, more preferably at least about 90% and most preferably at least about 95% homologous to the sequence

(SEQ ID NO:676) of S57296_1_P85 (SEQ ID NO:544)

VCYGLGMEHLREVRAVTSANIQEFAGCKKIFGSLAFLPESFDGVSLCQQA
GVQWYDLGSLQPLPPGFKQFSCLSLLSSWDYRDPASNTAPLQPEQLQVFE
TLEEITGYLYISAWPDSLPDLSVFQNLQVIRGRILHNGAYSLTLQGLGIS
WLGLRSLRELGSGLALIHHNTHLCFVHTVPWDQLFRNPHQALLHTANRPE
DECGKTGSPVCALPICQHTAVPRGPWQQRSWTCADCPSLCTLLDSAQLWL
AWPLGMASLAGSYLPWHPSLPLCF.

An isolated chimeric polypeptide encoding for S57296_{—}1_P97 (SEQ ID NO:545), comprising a first amino acid sequence being at least 90% or preferably at least about 95% homologous to amino acids 1-342 of Q9UK79_HUMAN (SEQ ID NO:534), which also corresponds to amino acids 1-342 of S57296_{—}1_P97 (SEQ ID NO:545), and a second amino acid sequence being at least 70%, optionally at least 80%, preferably at least 85%, more preferably at least 90% and most preferably at least 95% homologous to a polypeptide having the sequence QPPTLPRSSQSSSKCLRLWKRSQVTYTSQHGRTACLTSASSRTCK (SEQ ID NO: 677) corresponding to amino acids 343-387 of S57296_{—}1_P97 (SEQ ID NO:545), wherein said first amino acid sequence and second amino acid sequence are contiguous and in a sequential order.

An isolated polypeptide encoding for an edge portion of S57296_{—}1_P97 (SEQ ID NO:545), comprising an amino acid sequence being at least 70%, optionally at least about 80%, preferably at least about 85%, more preferably at least about 90% and most preferably at least about 95% homologous to the sequence QPPTLPRSSQSSSKCLRLWKRSQVTYTSQHGRTACLTSASSRTCK (SEQ ID NO: 677) of S57296_{—}1_P97 (SEQ ID NO:545).

An isolated chimeric polypeptide encoding for S57296_{—}1_P125 (SEQ ID NO:546), comprising a first amino acid sequence being at least 90% or preferably at least about 95% homologous to amino acids 1-648 of ERB2_HUMAN (SEQ ID NO:538), which also corresponds to amino acids 1-648 of S57296_{—}1_P125 (SEQ ID NO:546), and a second amino acid sequence being at least 70%, optionally at least 80%, preferably at least 85%, more preferably at least 90% and most preferably at least 95% homologous to a polypeptide having the sequence RLAWTPGCTLHCPSLPHWMLGGHCCREGTP (SEQ ID NO: 678) corresponding to amino acids 649-678 of S57296_{—}1_P125 (SEQ ID NO:546), wherein said first amino acid sequence and second amino acid sequence are contiguous and in a sequential order.

An isolated polypeptide encoding for an edge portion of S57296_{—}1_P125 (SEQ ID NO:546), comprising an amino acid sequence being at least 70%, optionally at least about 80%, preferably at least about 85%, more preferably at least about 90% and most preferably at least about 95% homologous to the sequence RLAWTPGCTLHCPSLPHWMLGGHCCREGTP (SEQ ID NO: 678) of S57296_{—}1_P125 (SEQ ID NO:546).

An isolated chimeric polypeptide encoding for S57296_{—}1_P125 (SEQ ID NO:546), comprising a first amino acid sequence being at least 90% or preferably at least about 95% homologous to amino acids 1-648 of NP_{—}004439 (SEQ ID NO:540), which also corresponds to amino acids 1-648 of S57296_{—}1_P125 (SEQ ID NO:546), and a second amino acid sequence being at least 70%, optionally at least 80%, preferably at least 85%, more preferably at least 90% and most preferably at least 95% homologous to a polypeptide having the sequence RLAWTPGCTLHCPSLPHWMLGGHCCREGTP (SEQ ID NO: 678) corresponding to amino acids 649-678 of S57296_{—}1_P125 (SEQ ID NO:546), wherein said first amino acid sequence and second amino acid sequence are contiguous and in a sequential order.

An isolated polypeptide encoding for an edge portion of S57296_{—}1_P125 (SEQ ID NO:546), comprising an amino acid sequence being at least 70%, optionally at least about 80%, preferably at least about 85%, more preferably at least about 90% and most preferably at least about 95% homologous to the sequence RLAWTPGCTLHCPSLPHWMLGGHCCREGTP (SEQ ID NO: 678) of S57296_{—}1_P125 (SEQ ID NO:546).

An isolated chimeric polypeptide encoding for S57296_{—}1_P125 (SEQ ID NO:546), comprising a first amino acid sequence being at least 70%, optionally at least 80%, preferably at least 85%, more preferably at least 90% and most preferably at least 95%, homologous to a polypeptide having the sequence MELAALCRWGLLLALLPPGAASTQVCTGTD (SEQ ID NO: 673) corresponding to amino acids 1-30 of S57296_{—}1_P125 (SEQ ID NO:546), a second amino acid sequence being at least 70%, optionally at least 80%, preferably at least 85%, more preferably at least 90% and most preferably at least 95%, homologous to a polypeptide having the sequence MKLRLPASPETHLDMLRHLYQGCQVVQGNLELTYLPTNASLSFLQDIQEVQGYVLIAHNQVRQVP LQRLRIVRGTQLFEDNYALAVLDNGDPLNNTTPVTGASPGGLRELQLRSLTEILKGGVLIQRNPQL CYQDTILWKDIFHKNNQLALTLIDTNRSRACHPCSPMCKGSRCWGESSEDCQSLTRTVCAGGCAR CKGPLPTDCCHEQCAAGCTGPKHSDCLACLHFNHSGICELHCPALVTYNTDTFESMPNPEGRYTFG ASCVTACPYNYLSTDVGSCTLVCPLHNQEVTAEDGTQRCEKCSKPCARVCYGLGMEHLREVRAV TSANIQEFAGCKKIFGSLAFLPESFDGDPASNTAPLQPEQLQVFETLEEITGYLYISAWPDSLPDLSV FQNLQVIRGRILHNGAYSLTLQGLGISWLGLRSLRELGSGLALIHHNTHLCFVHTVPWDQLFRNPH QALLHTANRPEDECVGEGLACHQLCARGHCWGPGPTQCVNCSQFLRGQECVEECRVLQGLPREY VNARHCLPCHPECQPQNGSVTCFGPEADQCVACAHYKDPPFCVARCPSGVKPDLSYMPIWKFPDE EGACQPCPINCTHSCVDLDDKGCPAEQRARLAWTPGCTLHCPSLPHWM corresponding to amino acids 649-678 of S57296_{—}1_P125 (SEQ ID NO:546), and a third amino acid sequence being at least 90% or preferably at least about 95% homologous to amino acids 1-618 of NP_{—}001005862 (SEQ ID NO:539), which also corresponds to amino acids 31-648 of S57296_{—}1_P125 (SEQ ID NO:546), wherein said first amino acid sequence, second amino acid sequence and third amino acid sequence are contiguous and in a sequential order.

An isolated polypeptide encoding for a head of S57296_{—}1_P125 (SEQ ID NO:546), comprising a polypeptide being at least 70%, optionally at least about 80%, preferably at least about 85%, more preferably at least about 90% and most preferably at least about 95% homologous to the sequence MELAALCRWGLLLALLPPGAASTQVCTGTD (SEQ ID NO: 673) of S57296_{—}1_P125 (SEQ ID NO:546).

An isolated chimeric polypeptide encoding for S57296_{—}1_P125 (SEQ ID NO:546), comprising a first amino acid sequence being at least 90% or preferably at least about 95% to amino acids 1-340 of Q9UK79_HUMAN (SEQ ID NO:534), which also corresponds to amino acids 1-340 of S57296_{—}1_P125 (SEQ ID NO:546), and a second amino acid sequence being at least 70%, optionally at least 80%, preferably at least 85%, more preferably at least 90% and most preferably at least 95% homologous to a polypeptide having the sequence VCYGLGMEHLREVRAVTSANIQEFAGCKKIFGSLAFLPESFDGDPASNTAPLQPEQLQVFETLEEIT GYLYISAWPDSLPDLSVFQNLQVIRGRILHNGAYSLTLQGLGISWLGLRSLRELGSGLALIHHNTHL CFVHTVPWDQLFRNPHQALLHTANRPEDECVGEGLACHQLCARGHCWGPGPTQCVNCSQFLRGQ ECVEECRVLQGLPREYVNARHCLPCHPECQPQNGSVTCFGPEADQCVACAHYKDPPFCVARCPSG VKPDLSYMPIWKFPDEEGACQPCPINCTHSCVDLDDKGCPAEQRARLAWTPGCTLHCPSLPHWML GGHCCREGTP (SEQ ID NO: 680) corresponding to amino acids 341-678 of S57296_{—}1_P125 (SEQ ID NO:546), wherein said first amino acid sequence and second amino acid sequence are contiguous and in a sequential order.

An isolated polypeptide encoding for an edge portion of S57296_{—}1_P125 (SEQ ID NO:546), comprising an amino acid sequence being at least 70%, optionally at least about 80%, preferably at least about 85%, more preferably at least about 90% and most preferably at least about 95% homologous to the sequence

(SEQ ID NO:546)

VCYGLGMEHLREVRAVTSANIQEFAGCKKIFGSLAFLPESFDGDPASNTA
PLQPEQLQVFETLEEITGYLYISAWPDSLPDLSVFQNLQVIRGRILHNGA
YSLTLQGLGISWLGLRSLRELGSGLALIHHNTHLCFVHTVPWDQLFRNPH
QALLHTANRPEDECVGEGLACHQLCARGHCWGPGPTQCVNCSQFLRGQEC
VEECRVLQGLPREYVNARHCLPCHPECQPQNGSVTCFGPEADQCVACAHY
KDPPFCVARCPSGVKPDLSYMPIWKFPDEEGACQPCPINCTHSCVDLDDK
GCPAEQRARLAWTPGCTLHCPSLPHWMLGGHCCREGTP
(SEQ ID NO: 680) of S57296_1_P125.

An isolated chimeric polypeptide encoding for S57296_{—}1_P127 (SEQ ID NO:547), comprising a first amino acid sequence being at least 90% or preferably at least about 95% homologous to amino acids 1-383 of ERB2_HUMAN (SEQ ID NO:538), which also corresponds to amino acids 1-383 of S57296_{—}1_P127 (SEQ ID NO:547), a second amino acid sequence being at least 70%, optionally at least 80%, preferably at least 85%, more preferably at least 90% and most preferably at least 95% homologous to a polypeptide having the sequence VSLCQQAGVQWYDLGSLQPLPPGFKQFSCLSLLSSWDYR (SEQ ID NO: 672) corresponding to amino acids 384-422 of S57296_{—}1_P127 (SEQ ID NO:547), a third amino acid sequence being at least 90% or preferably at least about 95% to amino acids 384-648 of ERB2_HUMAN (SEQ ID NO:538), which also corresponds to amino acids 423-687 of S57296_{—}1_P127 (SEQ ID NO:547), and a fourth amino acid sequence being at least 70%, optionally at least 80%, preferably at least 85%, more preferably at least 90% and most preferably at least 95% homologous to a polypeptide having the sequence RLAWTPGCTLHCPSLPHWMLGGHCCREGTP (SEQ ID NO: 678) corresponding to amino acids 688-717 of S57296_{—}1_P127 (SEQ ID NO:547), wherein said first amino acid sequence, second amino acid sequence, third amino acid sequence and fourth amino acid sequence are contiguous and in a sequential order.

An isolated polypeptide encoding for an edge portion of S57296_{—}1_P127 (SEQ ID NO:547), comprising an amino acid sequence being at least 70%, optionally at least about 80%, preferably at least about 85%, more preferably at least about 90% and most preferably at least about 95% homologous to the sequence VSLCQQAGVQWYDLGSLQPLPPGFKQFSCLSLLSSWDYR (SEQ ID NO: 672) of S57296_{—}1_P127 (SEQ ID NO:547).

C. An isolated polypeptide encoding for an edge portion of S57296_{—}1_P127 (SEQ ID NO:547), comprising an amino acid sequence being at least 70%, optionally at least about 80%, preferably at least about 85%, more preferably at least about 90% and most preferably at least about 95% homologous to the sequence RLAWTPGCTLHCPSLPHWMLGGHCCREGTP (SEQ ID NO: 678) of S57296_{—}1_P127 (SEQ ID NO:547).

An isolated chimeric polypeptide encoding for S57296_{—}1_P127 (SEQ ID NO:547), comprising a first amino acid sequence being at least 90% or preferably at least about 95% homologous to amino acids 1-383 of NP_{—}004439 (SEQ ID NO:540), which also corresponds to amino acids 1-383 of S57296_{—}1_P127 (SEQ ID NO:547), a second amino acid sequence being at least 70%, optionally at least 80%, preferably at least 85%, more preferably at least 90% and most preferably at least 95% homologous to a polypeptide having the sequence VSLCQQAGVQWYDLGSLQPLPPGFKQFSCLSLLSSWDYR (SEQ ID NO: 672) corresponding to amino acids 384-422 of S57296_{—}1_P127 (SEQ ID NO:547), a third amino acid sequence being at least 90% or preferably at least about 95% homologous to amino acids 384-648 of NP_{—}004439 (SEQ ID NO:540), which also corresponds to amino acids 423-687 of S57296_{—}1_P127 (SEQ ID NO:547), and a fourth amino acid sequence being at least 70%, optionally at least 80%, preferably at least 85%, more preferably at least 90% and most preferably at least 95% homologous to a polypeptide having the sequence RLAWTPGCTLHCPSLPHWMLGGHCCREGTP (SEQ ID NO: 678) corresponding to amino acids 688-717 of S57296_{—}1_P127 (SEQ ID NO:547), wherein said first amino acid sequence, second amino acid sequence, third amino acid sequence and fourth amino acid sequence are contiguous and in a sequential order.

An isolated polypeptide encoding for an edge portion of S57296_{—}1_P127 (SEQ ID NO:547), comprising an amino acid sequence being at least 70%, optionally at least about 80%, preferably at least about 85%, more preferably at least about 90% and most preferably at least about 95% homologous to the sequence VSLCQQAGVQWYDLGSLQPLPPGFKQFSCLSLLSSWDYR (SEQ ID NO: 672) of S57296_{—}1_P127 (SEQ ID NO:547).

An isolated polypeptide encoding for an edge portion of S57296_{—}1_P127 (SEQ ID NO:547), comprising an amino acid sequence being at least 70%, optionally at least about 80%, preferably at least about 85%, more preferably at least about 90% and most preferably at least about 95% homologous to the sequence RLAWTPGCTLHCPSLPHWMLGGHCCREGTP (SEQ ID NO: 678) of S57296_{—}1_P127 (SEQ ID NO:547).

An isolated chimeric polypeptide encoding for S57296_{—}1_P127 (SEQ ID NO:547), comprising a first amino acid sequence being at least 70%, optionally at least 80%, preferably at least 85%, more preferably at least 90% and most preferably at least 95%, homologous to a polypeptide having the sequence MELAALCRWGLLLALLPPGAASTQVCTGTD (SEQ ID NO: 673) corresponding to amino acids 1-30 of S57296_{—}1_P127 (SEQ ID NO:547), a second amino acid sequence being at least 70%, optionally at least 80%, preferably at least 85%, more preferably at least 90% and most preferably at least 95% homologous to a polypeptide having the sequence MKLRLPASPETHLDMLRHLYQGCQVVQGNLELTYLPTNA (SEQ ID NO: 674) corresponding to amino acids 384-422 of S57296_{—}1_P127 (SEQ ID NO:547), a third amino acid sequence being at least 90% or preferably at least about 95% homologous to amino acids 1-353 of NP_{—}001005862 (SEQ ID NO:539), which also corresponds to amino acids 31-383 of S57296_{—}1_P127 (SEQ ID NO:547), a fourth amino acid sequence being at least 70%, optionally at least 80%, preferably at least 85%, more preferably at least 90% and most preferably at least 95%, homologous to a polypeptide having the sequence DPASNTAPLQPEQLQVFETLEEITGYLYISAWPDSLPDLSVFQNLQVIRGRILHNGAYSLTLQGLGIS WLGLRSLRELGSGLALIHHNTHLCFVHTVPWDQLFRNPHQALLHTANRPEDECVGEGLACHQLC ARGHCWGPGPTQCVNCSQFLRGQECVEECRVLQGLPREYVNARHCLPCHPECQPQNGSVTCFGPE ADQCVACAHYKDPPFCVARCPSGVKPDLSYMPIWKFPDEEGACQPCPINCTHSCVDLDDKGCPAE QRARLAWTPGCTLHCPSLPHWMLGGHCCREGTP corresponding to amino acids 688-717 of S57296_{—}1_P127 (SEQ ID NO:547), and a fifth amino acid sequence being at least 90% homologous to amino acids 354-618 of NP_{—}001005862 (SEQ ID NO:539), which also corresponds to amino acids 423-687 of S57296_{—}1_P127 (SEQ ID NO:547), wherein said first amino acid sequence, second amino acid sequence, third amino acid sequence, fourth amino acid sequence and fifth amino acid sequence are contiguous and in a sequential order.

An isolated polypeptide encoding for a head of S57296_{—}1_P127 (SEQ ID NO:547), comprising a polypeptide being at least 70%, optionally at least about 80%, preferably at least about 85%, more preferably at least about 90% and most preferably at least about 95% homologous to the sequence MELAALCRWGLLLALLPPGAASTQVCTGTD (SEQ ID NO: 673) of S57296_{—}1_P127 (SEQ ID NO:547).

An isolated polypeptide encoding for an edge portion of S57296_{—}1_P127 (SEQ ID NO:547), comprising an amino acid sequence being at least 70%, optionally at least about 80%, preferably at least about 85%, more preferably at least about 90% and most preferably at least about 95% homologous to the sequence MKLRLPASPETHLDMLRHLYQGCQVVQGNLELTYLPTNA (SEQ ID NO: 674) of S57296_{—}1_P127 (SEQ ID NO:547).

An antibody capable of specifically binding to an epitope (antigen determinant) of an amino acid sequence as described herein. An antibody that “specifically binds” to a particular antigen determinant, for example, an antigen determinant present on a variant protein polypeptide of the invention, preferably does not substantially recognize or bind to other molecules in a sample, such as a biological sample. In some embodiments, binding of a variant protein antibody is at least about 2, preferably at least about 5, and more preferably at least about 10-fold greater than binding observed under the same reaction conditions to a molecule that does not include an antigen determinant present on a variant protein.

An antibody capable of specifically binding to an epitope of an amino acid sequence as described above, optionally wherein said amino acid sequence corresponds to a bridge, edge portion, tail, or head as in any of the previous claims, also optionally wherein said antibody is capable of differentiating between a splice variant having said epitope and a corresponding known protein.

A kit for detecting a Marker-detectable disease, comprising a kit detecting specific expression of a splice variant as described herein.

Optionally, the kit comprises a NAT-based technology; optionally and preferably, the kit further comprises at least one nucleotide probe or primer, alternatively and optionally this kit comprises at least one primer pair capable of selectively hybridizing to a nucleic acid sequence as described herein; alternatively and optionally, said kit further comprises at least one oligonucleotide capable of selectively hybridizing to a nucleic acid sequence according to any of the above claims.

Alternatively and optionally, the kit comprises an antibody according to any of the above claims (optionally and preferably, the kit further comprises at least one reagent for performing an ELISA or a Western blot.

A method for detecting a Marker-detectable disease, comprising detecting specific expression of a splice variant as described herein; optionally the marker-detectable disease is cluster N56180 marker-detectable disease, cluster S67314 marker-detectable disease, cluster HUMNATPEP marker-detectable disease, cluster HUMCDDANF marker-detectable disease, cluster HSACMHCP marker-detectable disease, cluster HSCREACT marker-detectable disease, or cluster Z3624 marker-detectable disease, and is selected from the group consisting of variety of cardiac diseases. According to preferred embodiments of the present invention, cardiac disease and/or pathology and/or condition and/or disorder may comprise one or more of Myocardial infarct, acute coronary syndrome, angina pectoris (stable and unstable), cardiomyopathy, myocarditis, congestive heart failure or any type of heart failure, the detection of reinfarction, the detection of success of thrombolytic therapy after Myocardial infarct, Myocardial infarct after surgery, assessing the size of infarct in Myocardial infarct, the differential diagnosis of heart related conditions from lung related conditions (as pulmonary embolism), the differential diagnosis of Dyspnea, and cardiac valves related conditions.

Alternatively and optionally, the marker-detectable disease is stroke and a marker comprises one or more of IL-1ra, C-reactive protein (CRP) or variants thereof as described herein with regard to cluster HSCREACT, von Willebrand factor (vWF), vascular endothelial growth factor (VEGF) or variants thereof as described with regard to U.S. Pat. No. 6,783,954 (previously incorporated by reference), matrix metalloprotease-9 (MMP-9), neural cell adhesion molecule (NCAM) or variants thereof as described with regard to PCT Application No. WO 01/29215 (incorporated by reference as if fully set forth herein), BNP or variants thereof as described herein with regard to cluster HUMNATPEP, markers from cluster N56180, S67314, HUMCDDANF and/or HSACMHCP, and caspase-3, or markers related thereto, or combinations thereof as described herein. Stroke optionally comprises stroke or neural tissue injury, or any type of cerebrovascular accident. Stroke optionally and preferably comprises ischemic stroke, hemorrhagic stroke or transient ischemic attacks. Ischemic stroke encompasses thrombotic, embolic, lacunar and hypoperfusion types of strokes. Stroke as a marker-detectable disease may also optionally comprise one or more of brain trauma, in case it is unclear whether accompanied by stroke or not; migraine as a symptom; bleeding in any part of the brain or inside the skull that cause or didn't cause damage to brain tissue; tumor. Such markers may help determine: the time of stroke; the type of stroke; the extent of tissue damage as a result of the stroke; response to immediate treatments that are meant to alleviate the extent of stroke and brain damage, when available.

With regard to stroke, according to preferred embodiments of the present invention, a marker as described herein or a panel may optionally and preferably provide diagnosis of stroke and indication if an ischemic stroke has occurred; diagnosis of stroke and indication if a hemorrhagic stroke has occurred; diagnosis of stroke, indication if an ischemic stroke has occurred, and indication if a hemorrhagic stroke has occurred; diagnosis of stroke and prognosis of a subsequent cerebral vasospasm; and diagnosis of stroke, indication if a hemorrhagic stroke has occurred, and prognosis of a subsequent cerebral vasospasm.

According to other optional embodiments of the present invention, there are provided methods of identifying a patient at risk for cerebral vasospasm. Such methods preferably comprise comparing an amount of one or more marker(s) predictive of a subsequent cerebral vasospasm in a test sample from a patient diagnosed with a subarachnoid hemorrhage. Such markers may be one or more markers related to blood pressure regulation, markers related to inflammation, markers related to apoptosis, and/or specific markers of neural tissue injury.

Alternatively and optionally, the marker-detectable disease is cardiomyopathy and myocarditis, and/or related conditions as described herein, and a marker comprises a marker optionally selected from the group consisting of one or more variants in N56180, S67314, HUMNATPEP, HUMCDDANF, HSACMHCP, HSCREACT or Z36249 clusters, or combinations thereof.

Alternatively and optionally, the marker-detectable disease is acute and chronic inflammation, and/or CVS diseases, and a marker comprises one or more of N56180 variants, S67314 variants, HUMNATPEP variants, HUMCDDANF variants, HSACMHCP variants, HSCREACT variants and/or Z3624 variants, including for a spectrum of diseases where an inflammatory process plays a substantial role. Conditions that may be diagnosed by these markers or variants of them include but are not limited to the presence, risk and/or extent of the following: conditions that entail an inflammatory process that involves blood vessels including but not limited to hypercholesterolemia, diabetes, atherosclerosis, inflammation that involves blood vessels—whether acute or chronic including but not limited to the coronary arteries and blood vessels of the brain, myocardial infarction, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behçet syndrome, and their complications including but not limited to coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina; rheumatic/autoimmune diseases that involve systemic immune reaction including but not limited to rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus; acute and/or chronic infective processes that involve systemic immune reaction including but not limited to pneumonia, bacteremia, sepsis, pyelonephritis, cellulitis, osteomyelitis, meningitis and viral hepatitis; malignant and idiopathic processes that involve systemic immune reaction and/or proliferation of immune cells including but not limited to granulomatous disorders, Wegener's granulomatosis, lymphomatoid granulomatosis/polymorphic reticulosis, idiopathic midline granuloma, multiple myeloma, Waldenstrom's macroglobulinemia, Castleman's disease, amyloidosis, lymphoma, histiocytosis, renal cell carcinoma and paraneoplastic syndromes; conditions where CRP was shown to have a positive correlation with the presence of the condition including but not limited to weight loss, anorexia-cachexia syndrome, extent of disease, recurrence in advanced cancer, diabetes (types 1 & 2), obesity, hypertension, preterm delivery; conditions which have similar symptoms, signs and complications as the conditions above and where the differential diagnosis between them and the conditions above is of clinical importance including but not limited to: other (non vascular) causes of heart disease, renal disease and cerebral disease; other (non rheumatic) causes of arthropathy and musculoskeletal pain; other causes of non-specific symptoms and signs such as fever of unknown origin, loss of appetite, weight loss, nonspecific pains, breathing difficulties and anxiety.

Alternatively and optionally, the marker-detectable disease is congestive heart failure (CHF), and a marker comprises a marker optionally selected from the group consisting of one or more variants in N56180 variants, S67314 variants, HUMNATPEP variants, HUMCDDANF variants, HSACMHCP variants, HSCREACT variants, HSTGFB1 variants and/or Z3624 variants or combinations thereof. Other conditions that may be diagnosed by these markers or variants of them include but are not limited to the presence, risk and/or extent of the following: sudden cardiac death, from arrhythmia or any other heart related reason; rejection of a transplanted heart; conditions that lead to heart failure including but not limited to myocardial infarction, angina, arrhythmias, valvular diseases, atrial and/or ventricular septal defects; conditions that cause atrial and or ventricular wall volume overload, including but not limited to systemic arterial hypertension, pulmonary hypertension and pulmonary embolism; conditions which have similar clinical symptoms as heart failure and as states that cause atrial and or ventricular pressure-overload, where the differential diagnosis between these conditions to the latter is of clinical importance including but not limited to breathing difficulty and/or hypoxia due to pulmonary disease, anemia or anxiety.

Alternatively and optionally, the marker-detectable disease is cluster HSSTROL3 marker-detectable disease and is selected from the group consisting of variety of cancers, including but not limited to colon cancer, breast cancer, ovarian cancer, prostate cancer, or lung cancer.

With regard to lung cancer, the disease (and/or diagnostic method to be performed) optionally and preferably comprises one or more of invasive or metastatic lung cancer; squamous cell lung carcinoma, lung adenocarcinoma, carcinoid, small cell lung cancer or non-small cell lung cancer; detection of overexpression in lung metastasis (vs. primary tumor); detection of overexpression in lung cancer, preferably non small cell lung cancer, preferably adenocarcinoma, squamous cell cancer or carcinoid, or large cell carcinoma; identification of a metastasis of unknown origin which originated from a primary lung cancer; assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors; distinguishing between different types of lung cancer, therefore potentially affect treatment choice (e.g. small cell vs. non small cell tumors); analysis of unexplained dyspnea and/or chronic cough and/or hemoptysis; differential diagnosis of the origin of a pleural effusion; diagnosis of conditions which have similar symptoms, signs and complications as lung cancer and where the differential diagnosis between them and lung cancer is of clinical importance including but not limited to: non-malignant causes of lung symptoms and signs, including but not limited to: lung lesions and infiltrates, wheeze, stridor, tracheal obstruction, esophageal compression, dysphagia, recurrent laryngeal nerve paralysis, hoarseness, phrenic nerve paralysis with elevation of the hemidiaphragm and Horner syndrome; or detecting a cause of any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

With regard to breast cancer, the disease (and/or diagnostic method to be performed) optionally and preferably comprises one or more of invasive or metastatic breast cancer; determining a probable outcome; detecting breast cancer in patients with age above 55 and/or patients with an age below 45; identification of a metastasis of unknown origin which originated from a primary breast cancer tumor; assessing lymphadenopathy, and in particular axillary lymphadenopathy; distinguishing between different types of breast cancer, therefore potentially affect treatment choice (e.g. as HER-2); differentially diagnosing between a benign and malignant breast mass; as a tool in the assessment of conditions affecting breast skin (e.g. Paget's disease) and their differentiation from breast cancer; differential diagnosis of breast pain or discomfort resulting from either breast cancer or other possible conditions (e.g. mastitis, Mondors syndrome); non-breast cancer conditions which have similar symptoms, signs and complications as breast cancer and where the differential diagnosis between them and breast cancer is of clinical importance including but not limited to: abnormal mammogram and/or nipple retraction and/or nipple discharge due to causes other than breast cancer, including but not limited to benign breast masses, melanoma, trauma and technical and/or anatomical variations; determining a cause of any condition suggestive of a malignant tumor including but not limited to anorexia, cachexia, weight loss, fever, hypercalcemia, paraneoplastic syndrome; or determining a cause of lymphadenopathy, weight loss and other signs and symptoms associated with breast cancer but originate from diseases different from breast cancer including but not limited to other malignancies, infections and autoimmune diseases.

With regard to prostate cancer, the disease (and/or diagnostic method to be performed) optionally and preferably comprises one or more of invasive or metastatic prostate cancer.

With regard to colon cancer, the disease (and/or diagnostic method to be performed) optionally and preferably comprises one or more of invasive or metastatic colon cancer.

With regard to ovarian cancer, the disease (and/or diagnostic method to be performed) optionally and preferably comprises one or more of invasive or metastatic ovarian cancer; correlating stage and malignant potential; identification of a metastasis of unknown origin which originated from a primary ovarian cancer, for example gastric carcinoma (such as Krukenberg tumor), breast cancer, colorectal carcinoma and pancreatic carcinoma; distinguishing between different types of ovarian cancer, therefore potentially affect treatment choice (e.g. discrimination between epithelial tumors and germ cell tumors); differential diagnosis between benign and malignant ovarian cysts; diagnosing a cause of infertility, particularly differential diagnosis of various causes thereof; detecting of one or more non-ovarian cancer conditions that may elevate serum levels of ovary related markers, including but not limited to: cancers of the endometrium, cervix, fallopian tubes, pancreas, breast, lung and colon; nonmalignant conditions such as pregnancy, endometriosis, pelvic inflammatory disease and uterine fibroids; diagnosing conditions which have similar symptoms, signs and complications as ovarian cancer and where the differential diagnosis between them and ovarian cancer is of clinical importance including but not limited to: non-malignant causes of pelvic mass, including, but not limited to: benign (functional) ovarian cyst, uterine fibroids, endometriosis, benign ovarian neoplasms and inflammatory bowel lesions; determining a cause of any condition suggestive of a malignant tumor including but not limited to anorexia, cachexia, weight loss, fever, hypercalcemia, skeletal or abdominal pain, paraneoplastic syndrome, or ascites.

With regard to a marker-detectable disease comprising colon cancer, breast cancer, ovarian cancer, prostate cancer, or lung cancer, optionally and preferably any condition or method of use described above is also suitable for any marker described below as being diagnostically useful for that marker-detectable disease.

Alternatively and optionally, the marker-detectable disease is cluster HUMGRP5E marker-detectable, cluster T94936 marker-detectable, or cluster HSTGFB1 marker-detectable disease and is selected from the group consisting of variety of cancers, including but not limited to colon cancer, breast cancer, ovarian cancer, lung cancer; and colon, breast, ovarian, and lung cancer invasion and metastasis.

Alternatively and optionally, the marker-detectable disease is cluster S57296 marker-detectable disease and is selected from the group consisting of variety of cancers, including but not limited to breast cancer, ovarian cancer, lung cancer; and breast, ovarian, and lung cancer invasion and metastasis.

Alternatively and optionally, the marker-detectable disease is cluster M78530 marker-detectable disease and is selected from the group consisting of variety of cancers, including but not limited to ovarian cancer and ovarian cancer invasion and metastasis.

Detecting specific expression is optionally performed with a NAT-based technology (optionally comprising at least one nucleotide probe or primer), and/or with an immunoassay (optionally comprising an antibody according to any of the above embodiments).

There is also optionally provided a biomarker capable of detecting Marker-detectable disease, comprising any of the above nucleic acid sequences or a fragment thereof, or any of the above amino acid sequences or a fragment thereof.

There is also optionally provided a method for screening for variant-detectable disease, comprising detecting cells affected by a Marker-detectable disease with a biomarker or an antibody or a method or assay according to any of the above embodiments.

There is also optionally provided a method for screening for a disease, comprising detecting cells affected by the disease using a marker selected from the group consisting of:
a. an amino acid sequence selected from the group consisting of SEQ ID NOs:84-90, 114-117, 139-141, 165-166, 239-244, 317-322, 364-369, 401-404, 427-429, 463-468, 542-547, 579-582, 619-621 or a homologue or fragment thereof;
b. an amino acid sequence corresponding to a bridge, edge portion, tail, or head having an amino acid sequence selected from the group consisting of SEQ ID NOs:628-684 or a homologue or fragment thereof;
c. a polynucleotide having a sequence selected from the group consisting of SEQ ID NOs:54-60, 100-103, 127-130, 151-153, 167-173, 251-260, 338-345, 388-392, 411-413, 433-438, 478-483, 564-567, 592-594 or a homologue or fragment thereof;
d. a polynucleotide comprising a node having a sequence selected from the group consisting of SEQ ID NOs:61-82, 104-111, 131-137, 154-163, 174-234, 261-315, 346-362, 393-399, 414-425, 439-462, 484-533, 568-578, 595-615;
e. an antibody capable of specifically binding to at least one epitope of an amino acid sequence selected from the group consisting of SEQ ID NOs: 84-90, 114-117, 139-141, 165-166, 239-244, 317-322, 364-369, 401-404, 427-429, 463-468, 542-547, 579-582, 619-621, 628-684,
f. an oligonucleotide having a sequence selected from the group consisting of SEQ ID NOs:10-26, 93, 96, 99, 120, 123, 126, 144, 147, 150, 247, 250, 325, 328, 331, 334, 337, 370, 375, 378, 381, 384, 387, 407, 410, 432, 471, 474, 477, 550, 552, 557, 560, 563, 585, 588, 591, 624, 627, 698;
g. a primer pair, comprising a pair of isolated oligonucleotides capable of amplifying an amplicon having a sequence selected from the group consisting of SEQ ID NOs: 93, 96, 99, 120, 123, 126, 144, 147, 150, 247, 250, 325, 328, 331, 334, 337, 370, 375, 378, 381, 384, 387, 407, 410, 432, 471, 474, 477, 550, 552, 557, 560, 563, 585, 588, 591, 624, 627, 698;
h. a primer pair, comprising a pair of isolated oligonucleotides having a sequence selected from the group consisting of SEQ ID NOs: 91-92, 94-95, 97-98, 121-122, 124-125, 142-143, 145-146, 148-149, 245-246, 248-249, 323-324, 326-327, 329-330, 332-333, 335-336, 371-372, 373-374, 376-377, 379-380, 382-383, 385-386, 405-406, 408-409, 430-431, 469-470, 472-473, 475-476, 548-549, 551 and 701, 553-554, 555-556, 558-559, 561-562, 583-584, 586-587, 589-590, 622-623, 699-700,
   to detect differential expression of a splice variant according to the invention.

There is also optionally provided a method for diagnosing a marker-detectable disease, comprising detecting cells affected by Marker-detectable disease with a biomarker or an antibody or a method or assay according to any of the above embodiments.

There is also optionally provided a method for diagnosing a disease, comprising detecting cells affected by the disease using a marker selected from the group consisting of:
a. an amino acid sequence selected from the group consisting of SEQ ID NOs:84-90, 114-117, 139-141, 165-166, 239-244, 317-322, 364-369, 401-404, 427-429, 463-468, 542-547, 579-582, 619-621 or a homologue or fragment thereof;
b. an amino acid sequence corresponding to a bridge, edge portion, tail, or head having an amino acid sequence selected from the group consisting of SEQ ID NOs:628-684 or a homologue or fragment thereof;
c. a polynucleotide having a sequence selected from the group consisting of SEQ ID NOs:54-60, 100-103, 127-130, 151-153, 167-173, 251-260, 338-345, 388-392, 411-413, 433-438, 478-483, 564-567, 592-594 or a homologue or fragment thereof;
d. a polynucleotide comprising a node having a sequence selected from the group consisting of SEQ ID NOs:61-82, 104-111, 131-137, 154-163, 174-234, 261-315, 346-362, 393-399, 414-425, 439-462, 484-533, 568-578, 595-615;
e. an antibody capable of specifically binding to at least one epitope of an amino acid sequence selected from the group consisting of SEQ ID NOs: 84-90, 114-117, 139-141, 165-166, 239-244, 317-322, 364-369, 401-404, 427-429, 463-468, 542-547, 579-582, 619-621, 628-684;
f. an oligonucleotide having a sequence selected from the group consisting of SEQ ID NOs:10-26, 93, 96, 99, 120, 123, 126, 144, 147, 150, 247, 250, 325, 328, 331, 334, 337, 370, 375, 378, 381, 384, 387, 407, 410, 432, 471, 474, 477, 550, 552, 557, 560, 563, 585, 588, 591, 624, 627, 698;
g. a primer pair, comprising a pair of isolated oligonucleotides capable of amplifying an amplicon having a sequence selected from the group consisting of SEQ ID NOs: 93, 96, 99, 120, 123, 126, 144, 147, 150, 247, 250, 325, 328, 331, 334, 337, 370, 375, 378, 381, 384, 387, 407, 410, 432, 471, 474, 477, 550, 552, 557, 560, 563, 585, 588, 591, 624, 627, 698;
h. a primer pair, comprising a pair of isolated oligonucleotides having a sequence selected from the group consisting of SEQ ID NOs: 91-92, 94-95, 97-98, 121-122, 124-125, 142-143, 145-146, 148-149, 245-246, 248-249, 323-324, 326-327, 329-330, 332-333, 335-336, 371-372, 373-374, 376-377, 379-380, 382-383, 385-386, 405-406, 408-409, 430-431, 469-470, 472-473, 475-476, 548-549, 551 and 701, 553-554, 555-556, 558-559, 561-562, 583-584, 586-587, 589-590, 622-623, 699-700,

to detect differential expression of a splice variant according to the invention.

There is also optionally provided a method for monitoring disease progression and/or treatment efficacy and/or relapse of Marker-detectable disease, comprising detecting cells affected by Marker-detectable disease with a biomarker or an antibody or a method or assay according to any of the above embodiments.

There is also optionally provided a method for monitoring disease progression or treatment efficacy or relapse of a disease, comprising detecting cells affected by the disease using a marker selected from the group consisting of:
a. an amino acid sequence selected from the group consisting of SEQ ID NOs:84-90, 114-117, 139-141, 165-166, 239-244, 317-322, 364-369, 401-404, 427-429, 463-468, 542-547, 579-582, 619-621 or a homologue or fragment thereof;
b. an amino acid sequence corresponding to a bridge, edge portion, tail, or head having an amino acid sequence selected from the group consisting of SEQ ID NOs:628-684 or a homologue or fragment thereof;
c. a polynucleotide having a sequence selected from the group consisting of SEQ ID NOs:54-60, 100-103, 127-130, 151-153, 167-173, 251-260, 338-345, 388-392, 411-413, 433-438, 478-483, 564-567, 592-594 or a homologue or fragment thereof;
d. a polynucleotide comprising a node having a sequence selected from the group consisting of SEQ ID NOs:61-82, 104-111, 131-137, 154-163, 174-234, 261-315, 346-362, 393-399, 414-425, 439-462, 484-533, 568-578, 595-615;
e. an antibody capable of specifically binding to at least one epitope of an amino acid sequence selected from the group consisting of SEQ ID NOs: 84-90, 114-117, 139-141, 165-166, 239-244, 317-322, 364-369, 401-404, 427-429, 463-468, 542-547, 579-582, 619-621, 628-684;
f. an oligonucleotide having a sequence selected from the group consisting of SEQ ID NOs:10-26, 93, 96, 99, 120, 123, 126, 144, 147, 150, 247, 250, 325, 328, 331, 334, 337, 370, 375, 378, 381, 384, 387, 407, 410, 432, 471, 474, 477, 550, 552, 557, 560, 563, 585, 588, 591, 624, 627, 698;
g. a primer pair, comprising a pair of isolated oligonucleotides capable of amplifying an amplicon having a sequence selected from the group consisting of SEQ ID NOs: 93, 96, 99, 120, 123, 126, 144, 147, 150, 247, 250, 325, 328, 331, 334, 337, 370, 375, 378, 381, 384, 387, 407, 410, 432, 471, 474, 477, 550, 552, 557, 560, 563, 585, 588, 591, 624, 627, 698;
h. a primer pair, comprising a pair of isolated oligonucleotides having a sequence selected from the group consisting of SEQ ID NOs: 91-92, 94-95, 97-98, 121-122, 124-125, 142-143, 145-146, 148-149, 245-246, 248-249, 323-324, 326-327, 329-330, 332-333, 335-336, 371-372, 373-374, 376-377, 379-380, 382-383, 385-386, 405-406, 408-409, 430-431, 469-470, 472-473, 475-476, 548-549, 551 and 701, 553-554, 555-556, 558-559, 561-562, 583-584, 586-587, 589-590, 622-623, 699-700,

to detect differential expression of a splice variant according to the invention.

There is also optionally provided a method of selecting a therapy for a marker-detectable disease, comprising detecting cells affected by a marker-detectable disease with a biomarker or an antibody or a method or assay according to any of the above embodiments and selecting a therapy according to said detection.

There is also optionally provided a method of selecting a therapy for a disease, comprising detecting cells affected by the disease using a marker selected from the group consisting of:
a. an amino acid sequence selected from the group consisting of SEQ ID NOs:84-90, 114-117, 139-141, 165-166, 239-244, 317-322, 364-369, 401-404, 427-429, 463-468, 542-547, 579-582, 619-621 or a homologue or fragment thereof;
b. an amino acid sequence corresponding to a bridge, edge portion, tail, or head having an amino acid sequence selected from the group consisting of SEQ ID NOs:628-684 or a homologue or fragment thereof;
c. a polynucleotide having a sequence selected from the group consisting of SEQ ID NOs:54-60, 100-103, 127-130, 151-153, 167-173, 251-260, 338-345, 388-392, 411-413, 433-438, 478-483, 564-567, 592-594 or a homologue or fragment thereof;
d. a polynucleotide comprising a node having a sequence selected from the group consisting of SEQ ID NOs:61-82, 104-111, 131-137, 154-163, 174-234, 261-315, 346-362, 393-399, 414-425, 439-462, 484-533, 568-578, 595-615;
e. an antibody capable of specifically binding to at least one epitope of an amino acid sequence selected from the group consisting of SEQ ID NOs: 84-90, 114-117, 139-141, 165-166, 239-244, 317-322, 364-369, 401-404, 427-429, 463-468, 542-547, 579-582, 619-621, 628-684;
f. an oligonucleotide having a sequence selected from the group consisting of SEQ ID NOs:10-26, 93, 96, 99, 120, 123, 126, 144, 147, 150, 247, 250, 325, 328, 331, 334, 337, 370, 375, 378, 381, 384, 387, 407, 410, 432, 471, 474, 477, 550, 552, 557, 560, 563, 585, 588, 591, 624, 627, 698;
g. a primer pair, comprising a pair of isolated oligonucleotides capable of amplifying an amplicon having a sequence selected from the group consisting of SEQ ID NOs: 93, 96, 99, 120, 123, 126, 144, 147, 150, 247, 250, 325, 328, 331, 334, 337, 370, 375, 378, 381, 384, 387, 407, 410, 432, 471, 474, 477, 550, 552, 557, 560, 563, 585, 588, 591, 624, 627, 698;
h. a primer pair, comprising a pair of isolated oligonucleotides having a sequence selected from the group consisting of SEQ ID NOs: 91-92, 94-95, 97-98, 121-122, 124-125, 142-143, 145-146, 148-149, 245-246, 248-249, 323-324, 326-327, 329-330, 332-333, 335-336, 371-372, 373-374, 376-377, 379-380, 382-383, 385-386, 405-406, 408-409, 430-431, 469-470, 472-473, 475-476, 548-549, 551 and 701, 553-554, 555-556, 558-559, 561-562, 583-584, 586-587, 589-590, 622-623, 699-700,

to detect differential expression of a splice variant according to the invention and selecting a therapy according to said detection.

The method of any of the above claims may optionally be used when the marker-detectable disease is marker-detectable disease is cluster N56180 marker-detectable disease, cluster S67314 marker-detectable disease, cluster HUMNATPEP marker-detectable disease, cluster HUMCDDANF marker-detectable disease, cluster HSACMHCP marker-detectable disease, cluster HSCREACT marker-detectable disease, or cluster Z3624 marker-detectable disease, and is selected from the group consisting of variety of cardiac diseases. According to preferred embodiments of the present invention, cardiac disease and/or pathology and/or condition and/or disorder may comprise one or more of Myocardial infarct, acute coronary syndrome, angina pectoris (stable and unstable), cardiomyopathy, myocarditis, congestive heart failure or any type of heart failure, the detection of reinfarction, the detection of success of thrombolytic therapy after Myocardial infarct, Myocardial infarct after surgery, assessing the size of infarct in Myocardial infarct, the differential diagnosis of heart related conditions from lung related conditions (as pulmonary embolism), the differential diagnosis of Dyspnea, and cardiac valves related conditions.

Alternatively and optionally, the marker-detectable disease is cluster HUMGRP5E marker-detectable, cluster T94936 marker-detectable, or cluster HSTGFB1 marker-detectable disease and is selected from the group consisting of variety of cancers, including but not limited to colon cancer, breast cancer, ovarian cancer, lung cancer; and colon, breast, ovarian, and lung cancer invasion and metastasis.

Alternatively and optionally, the marker-detectable disease is cluster S57296 marker-detectable disease and is selected from the group consisting of variety of cancers, including but not limited to breast cancer, ovarian cancer, lung cancer; and breast, ovarian, and lung cancer invasion and metastasis.

Alternatively and optionally, the marker-detectable disease is cluster M78530 marker-detectable disease and is selected from the group consisting of variety of cancers, including but not limited to ovarian cancer and ovarian cancer invasion and metastasis.

According to preferred embodiments of the present invention, preferably any of the above nucleic acid and/or amino acid sequences further comprises any sequence having at least about 70%, preferably at least about 80%, more preferably at least about 90%, most preferably at least about 95% homology thereto.

Unless otherwise noted, all experimental data relates to variants of the present invention, named according to the segment being tested (as expression was tested through RT-PCR as described).

All nucleic acid sequences and/or amino acid sequences shown herein as embodiments of the present invention relate to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that oligonucleotide and polynucleotide, or peptide and polypeptide, may optionally be used interchangeably.

With regard to markers suitable for detecting cardiac disease (including but not limited to HSCREACT), according to preferred embodiments of the present invention, cardiac disease and/or pathology and/or condition and/or disorder may comprise one or more of Myocardial infarct, acute coronary syndrome, angina pectoris (stable and unstable), cardiomyopathy, myocarditis, congestive heart failure or any type of heart failure, the detection of reinfarction, the detection of success of thrombolytic therapy after Myocardial infarct, Myocardial infarct after surgery, assessing the size of infarct in Myocardial infarct, the differential diagnosis of heart related conditions from lung related conditions (as pulmonary embolism), the differential diagnosis of Dyspnea, and cardiac valves related conditions.

For these embodiments, there are provided novel markers for cardiac disease that are both sensitive and accurate. Biomolecular sequences (amino acid and/or nucleic acid sequences) uncovered using the methodology of the present invention and described herein can be efficiently utilized as tissue or pathological markers and/or as drugs or drug targets for treating or preventing a disease.

These markers are specifically released to the bloodstream under conditions of cardiac disease and/or cardiac pathology, including but not limited to cardiac damage, and/or are otherwise expressed at a much higher level and/or specifically expressed in heart. The method of the present invention identifies clusters (genes) which are characterized in that the transcripts are differentially expressed in heart muscle tissue compared with other normal tissues, preferably in comparison to skeletal muscle tissue. In acute conditions under which heart muscle tissue experiences hypoxia (with or without necrosis), intracellular proteins that are not normally secreted can leak through the cell membrane to the extracellular space. Therefore, heart muscle tissue differentially expressed proteins, as through analysis of EST expression, are potential acute heart damage markers.

Leakage of intracellular content can also occur in chronic damage to the heart muscle, therefore proteins selected according to this method are potential markers for chronic heart conditions. When a protein that is differentially expressed in heart muscle is secreted, it is even more likely to be useful as a chronic heart damage marker, since secretion implies that the protein has a physiological role exterior to the cell, and therefore may be used by the heart muscle to respond to the chronic damage. This rationale is empirically supported by the non-limiting examples of the proteins BNP (brain natriuretic peptide) and ANF (atrial natriuretic factor), which are differentially expressed heart muscle proteins that are secreted and which were shown to be markers for congestive heart failure. In addition, BNP and ANF are not only differentially expressed in heart tissue, they are also overexpressed dramatically (hundreds of times greater expression) when heart failure occurs. Other heart specific secreted proteins might present similar overexpression in chronic damage.

Optionally and preferably, the markers described herein are overexpressed in heart as opposed to muscle, as described in greater detail below. The measurement of these markers, alone or in combination, in patient samples provides information that the diagnostician can correlate with a probable diagnosis of cardiac disease and/or cardiac pathology, including but not limited to cardiac damage.

The present invention therefore also relates to diagnostic assays for cardiac disease and/or cardiac pathology, including but not limited to cardiac damage, and methods of use of such markers for detection of cardiac disease and/or cardiac pathology, including but not limited to cardiac damage (alone or in combination), optionally and preferably in a sample taken from a subject (patient), which is more preferably some type of blood sample.

The present invention therefore also relates to diagnostic assays for cardiac disease and/or cardiac pathology, including but not limited to cardiac damage, and methods of use of such markers for detection of cardiac disease and/or cardiac pathology, including but not limited to cardiac damage (alone or in combination), optionally and preferably in a sample taken from a subject (patient), which is more preferably some type of blood sample.

The above description for cardiac pathology and diagnostic utilities optionally and preferably apply to markers (variants) according to the present invention that are described as being useful for cardiac related diagnostic utilities. More generally, such markers are useful for cardiovascular and cerebrovascular conditions, which are conditions that affect the vascular system, including various cardiovascular and cerebrovascular conditions. As described in greater detail below, these conditions may also optionally include stroke and various cardiomyopathies.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein. As used herein, the following terms have the meanings ascribed to them unless specified otherwise.

### BRIEF DESCRIPTION OF DRAWINGS

FIG. 1 shows schematic summary of the cancer biomarkers selection engine and the following wet validation stages.

FIG. 2 shows schematic illustration, depicting grouping of transcripts of a given cluster based on presence or absence of unique sequence regions.

FIG. 3 shows schematic presentation of the oligonucleotide based microarray fabrication.

FIG. 4 schematic summary of the oligonucleotide based microarray experimental flow.

FIG. 5 shows schematic summary of quantitative real-time PCR analysis.

FIG. 6 is a histogram showing differential expression for cluster N56180 in heart vs. other tissues.

FIG. 7 is a histogram showing expression of oligonucleotides in various tissues, including heart for cluster N56180 using Affimetrix probe 207317_s_at.

FIG. 8 is a histogram showing expression of Homo sapiens calsequestrin 2 N56180 transcripts which are detectable by amplicon as depicted in sequence name N56180 seg33-34 (SEQ ID NO:93) specifically in heart tissue.

FIG. 9 is a histogram showing expression of Calsequestrin 2 transcripts which are detectable by amplicon as depicted in sequence name N56180seg22 (SEQ ID NO: 96) specifically in heart tissue.

FIG. 10 is a histogram showing expression of Calsequestrin 2 transcripts which are detectable by amplicon as depicted in sequence name N56180seg6 (SEQ ID NO: 99) specifically in heart tissue.

FIG. 11 is a histogram showing differential expression for cluster S67314 in heart vs. other tissues.

FIG. 12 is a histogram showing expression of oligonucleotides in various tissues, including heart for cluster S67314 using Affimetrix probe 205738_s_at.

FIG. 13 is a histogram showing expression of oligonucleotides in various tissues, including heart for cluster S67314 using Affimetrix probe 214285_at.

FIG. 14 is a histogram showing expression of Fatty acid-binding protein (SEQ ID NO:112) transcripts which are detectable by amplicon as depicted in sequence name S67314seg11 (SEQ ID NO: 120) specifically in heart tissue.

FIG. 15 is a histogram showing expression of Fatty acid-binding protein (SEQ ID NO:112) S67314 transcripts, which are detectable by amplicon as depicted in sequence name S67314 seg15 (SEQ ID NO:123) specifically in heart tissue.

FIG. 16 is a histogram showing Expression of Fatty acid-binding protein (SEQ ID NO:112) S67314 transcripts which are detectable by amplicon as depicted in sequence name S67314seg4 (SEQ ID NO: 126) specifically in heart tissue.

FIG. 17 is a histogram showing differential expression for cluster HUMNATPEP in heart vs. other tissues.

FIG. 18 is a histogram showing expression of oligonucleotides in various tissues, including heart for cluster HUMNATPEP using Affimetrix probe 206801_at.

FIG. 19 is histogram showing expression of Homo sapiens natriuretic peptide precursor B (NPPB) HUMNATPEP transcripts which are detectable by amplicon as depicted in sequence name HUMNATPEP seg3-4WT (SEQ ID NO: 144) specifically in heart tissue.

FIG. 20 is a histogram showing expression of ANFB_HUMAN (SEQ ID NO:138) Natriuretic peptide HUMNATPEP transcripts which are detectable by amplicon as depicted in sequence name HUMNATPEP seg2 (SEQ ID NO: 147) specifically in heart tissue.

FIG. 21 is a histogram showing expression of ANFB_HUMAN (SEQ ID NO:138) Natriuretic peptides HUMNATPEP transcripts which are detectable by amplicon as depicted in sequence name HUMNATPEPseg5 (SEQ ID NO: 150) specifically in heart tissue.

FIG. 22 is a histogram showing differential expression for cluster HUMCDDANF in heart vs. other tissues.

FIG. 23 is a histogram showing expression of oligonucleotides in various tissues, including heart for cluster HUMCDDANF using Affimetrix probe 209957_s_at.

FIG. 24 is a histogram showing differential expression for cluster HSACMHCP in heart vs. other tissues.

FIG. 25 is a histogram showing expression of oligonucleotides in various tissues, including heart for cluster HSACMHCP using Affimetrix probe 204737_s_at.

FIG. 26 is a histogram showing expression of oligonucleotides in various tissues, including heart for cluster HSACMHCP using Affimetrix probe 216265_x_at.

FIG. 27 is a histogram showing expression of Homo sapiens myosin, heavy polypeptide 6, HSACMHCP transcripts which are detectable by amplicon as depicted in sequence name HSACMHCP seg106 (SEQ ID NO: 247) specifically in heart tissue.

FIG. 28 is a histogram showing expression of HSACMHCP transcripts which are detectable by amplicon as depicted in sequence name HSACMHCP seg46 (SEQ ID NO:250) specifically in heart tissue

FIGS. 29a and 29b are histograms showing on two different scales the expression of Homo sapiens C-reactive protein, pentraxin-related (CRP) HSCREACT transcripts which are detectable by amplicon as depicted in sequence name HSCREACT junc11-53F2R2 (SEQ ID NO:325) in different normal tissues.

FIGS. 30a and 30b are histograms showing on two different scales the expression of Homo sapiens C-reactive protein, pentraxin-related (CRP) HSCREACT transcripts which are detectable by amplicon as depicted in sequence name HSCREACT junc12-30F2R2 (SEQ ID NO:328) in different normal tissues.

FIGS. 31a and 31b are histograms showing on two different scales the expression of Homo sapiens C-reactive protein, pentraxin-related (CRP) HSCREACT transcripts which are detectable by amplicon as depicted in sequence name HSCREACT junc12-53F2R2 (SEQ ID NO:331) in different normal tissues.

FIG. 32 is a histogram showing expression of Homo sapiens C-reactive protein, pentraxin-related (CRP) HSCREACT transcripts which are detectable by amplicon as depicted in sequence name HSCREACT junc24-47F2R2 (SEQ ID NO:334) in different normal tissues.

FIG. 33 is a histogram showing expression of Homo sapiens C-reactive protein, pentraxin-related (CRP) HSCREACT transcripts which are detectable by amplicon as depicted in sequence name HSCREACT seg8-11 (SEQ ID NO: 337) in different normal tissues.

FIG. 34 is a histogram showing Cancer and cell-line vs. normal tissue expression for Cluster HSSTROL3, demonstrating overexpression in transitional cell carcinoma, epithelial malignant tumors, a mixture of malignant tumors from different tissues and pancreas carcinoma.

FIG. 35 is a histogram showing Expression of Homo sapiens matrix metalloproteinase 11 (stromelysin 3) (MMP11) HSSTROL3 transcripts which are detectable by amplicon as depicted in sequence name HSSTROL3 junc211-26 (SEQ ID NO:370) in normal and cancerous breast tissues.

FIG. 36 is a histogram showing expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in normal and cancerous colon tissues.

FIG. 37 is a histogram showing expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in normal and cancerous lung tissues.

FIG. 38 is a histogram showing expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in different normal tissues.

FIG. 39 is a histogram showing expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in normal and cancerous ovary tissues.

FIG. 40 is a histogram showing expression of Homo sapiens matrix metalloproteinase 11 (stromelysin 3) (MMP11) HSSTROL3 transcripts which are detectable by amplicon as depicted in sequence name HSSTROL3 junc21-27 (SEQ ID NO: 378) in normal and cancerous ovary tissues

FIG. 41 is a histogram showing expression of Stromelysin-3 precursor (SEQ ID NO:363) (EC 3.4.24.-) (Matrix metalloproteinase-11) (MMP-11) (ST3) (SL-3) HSSTROL3 transcripts which are detectable by amplicon as depicted in sequence name HSSTROL3 junc21-27 (SEQ ID NO: 378) in normal and cancerous breast tissues

FIG. 42 is a histogram showing expression of Homo sapiens matrix metalloproteinase 11 (stromelysin 3) (MMP11) HSSTROL3 transcripts which are detectable by amplicon as depicted in sequence name HSSTROL3 junc21-27 (SEQ ID NO: 378) in normal and cancerous colon tissues.

FIG. 43 is a histogram showing expression of Homo sapiens matrix metalloproteinase 11 (stromelysin 3) (MMP11) HSSTROL3 transcripts which are detectable by amplicon as depicted in sequence name HSSTROL3 junc21-27 (SEQ ID NO: 378) in normal and cancerous lung tissues.

FIG. 44 is a histogram showing expression of Stromelysin-3 precursor (SEQ ID NO:363) HSSTROL3 transcripts which are detectable by amplicon as depicted in sequence name HSSTROL3 junc21-27 (SEQ ID NO: 378) in different normal tissues.

FIG. 45 is a histogram showing expression of Homo sapiens matrix metalloproteinase 11 HSSTROL3 transcripts which are detectable by amplicon as depicted in sequence name HSSTROL3 seg20-21 (SEQ ID NO:381) in normal and cancerous colon tissues.

FIG. 46 is a histogram showing expression of Homo sapiens matrix metalloproteinase 11 (stromelysin 3) (MMP11) HSSTROL3 transcripts which are detectable by amplicon as depicted in sequence name HSSTROL seg20-21 (SEQ ID NO: 560) in normal and cancerous ovary tissues.

FIG. 47 is a histogram showing expression of Stromelysin-3 precursor (SEQ ID NO:363) HSSTROL3 transcripts which are detectable by amplicon as depicted in sequence name HSSTROL3 seg20-21 (SEQ ID NO:381) in normal and cancerous Prostate tissues.

FIG. 48 is a histogram showing expression of Homo sapiens matrix metalloproteinase 11 HSSTROL3 transcripts which are detectable by amplicon as depicted in sequence name HSSTROL3 seg20-21 (SEQ ID NO:381) in normal and cancerous lung tissues.

FIG. 49 is a histogram showing expression of Stromelysin-3 precursor (SEQ ID NO:363) HSSTROL3 transcripts which are detectable by amplicon as depicted in sequence name HSSTROL3 junc20-21 in normal and cancerous breast tissues.

FIG. 50 is a histogram showing Expression of Stromelysin-3 precursor (SEQ ID NO:363) HSSTROL3 transcripts which are detectable by amplicon as depicted in sequence name HSSTROL3 seg24 (SEQ ID NO:384) in normal and cancerous breast tissues

FIG. 51 is a histogram showing Expression of Stromelysin-3 precursor (SEQ ID NO:363) HSSTROL3 transcripts which are detectable by amplicon as depicted in sequence name HSSTROL3 seg24 (SEQ ID NO:384) in normal and cancerous lung tissues.

FIG. 52 is a histogram showing expression of Stromelysin-3 precursor (SEQ ID NO:363) HSSTROL3 transcripts which are detectable by amplicon as depicted in sequence name HSSTROL3 seg24 (SEQ ID NO:384) in different normal tissues.

FIG. 53 is a histogram showing expression of Stromelysin-3 precursor (SEQ ID NO:363) transcripts which are detectable by amplicon as depicted in sequence name HSSTROL3 seg24 (SEQ ID NO:384) in normal and cancerous Prostate tissues.

FIG. 54 is a histogram showing expression of Homo sapiens matrix metalloproteinase 11 HSSTROL3 transcripts which are detectable by amplicon as depicted in sequence name HSSTROL3 seg25 (SEQ ID NO:387) in normal and cancerous colon tissues.

FIG. 55 is a histogram showing expression of Stromelysin-3 precursor (SEQ ID NO:363) HSSTROL3 transcripts which are detectable by amplicon as depicted in sequence name HSSTROL3 seg25 (SEQ ID NO:387) in normal and cancerous breast tissues.

FIG. 56 is a histogram showing Expression of Homo sapiens matrix metalloproteinase 11 HSSTROL3 transcripts which are detectable by amplicon as depicted in sequence name HSSTROL3 seg25 (SEQ ID NO:387) in normal and cancerous lung tissues.

FIG. 57 is a histogram showing expression of Stromelysin-3 precursor (SEQ ID NO:363) transcripts which are detectable by amplicon as depicted in sequence name HSSTROL3 seg25 (SEQ ID NO:387) in normal and cancerous Prostate tissues.

FIG. 58 is a histogram showing Expression of Homo sapiens gastrin-releasing peptide (GRP) HUMGRP5E transcripts which are detectable by amplicon as depicted in sequence name HUMGRP5E seg2 (SEQ ID NO:407) in normal and cancerous lung tissues.

FIG. 59 is a histogram showing expression of Homo sapiens gastrin-releasing peptide (GRP) HUMGRP5E transcripts which are detectable by amplicon as depicted in sequence name HUMGRP5E seg2 (SEQ ID NO:407) in different normal tissues.

FIG. 60 is a histogram showing expression of Homo sapiens gastrin-releasing peptide (GRP) HUMGRP5E transcripts which are detectable by amplicon as depicted in sequence name HUMGRP5E seg2 (SEQ ID NO:407) in normal and cancerous breast tissues.

FIG. 61 is a histogram showing expression of Homo sapiens gastrin-releasing peptide (GRP) HUMGRP5E transcripts which are detectable by amplicon as depicted in sequence name HUMGRP5E seg2 (SEQ ID NO:407) in normal and cancerous ovary tissues.

FIG. 62 is a histogram showing expression of GRP_HUMAN (SEQ ID NO:400)—gastrin-releasing peptide (HUMGRP5E) transcripts, which are detectable by amplicon, as depicted in sequence name HUMGRP5Ejunc3-7 (SEQ ID NO: 410) in normal and cancerous breast tissues.

FIG. 63 is a histogram showing expression of GRP_HUMAN (SEQ ID NO:400)—gastrin-releasing peptide HUMGRP5E transcripts, which are detectable by amplicon as depicted in sequence name HUMGRP5E junc3-7 (SEQ ID NO: 410) in normal and cancerous ovary tissues.

FIG. 64 is a histogram showing expression of GRP_HUMAN (SEQ ID NO:400)—gastrin-releasing peptide HUMGRP5E transcripts, which are detectable by amplicon as depicted in sequence name HUMGRP5Ejunc3-7 (SEQ ID NO: 410) in normal and cancerous lung tissues.

FIG. 65 is a histogram showing expression of GRP_HUMAN (SEQ ID NO:400)—gastrin-releasing peptide HUMGRP5E transcripts, which are detectable by amplicon as depicted in sequence name HUMGRP5E junc3-7 (SEQ ID NO: 410) in different normal tissues.

FIG. 66 is a histogram showing expression of Homo sapiens breast cancer membrane protein 11 (BCMP11) T94936 transcripts which are detectable by amplicon as depicted in sequence name T94936 seg14 (SEQ ID NO: 563) in different normal tissues.

FIG. 67 is a histogram showing expression of Homo sapiens breast cancer membrane protein 11 (BCMP11) T94936 transcripts which are detectable by amplicon as depicted in sequence name T94936 seg14 (SEQ ID NO: 563) in normal and cancerous breast tissues.

FIG. 68 is a histogram showing expression of Homo sapiens breast cancer membrane protein 11 (BCMP11) T94936 transcripts which are detectable by amplicon as depicted in sequence name T94936 seg14 (SEQ ID NO: 563) in normal and cancerous ovary tissues.

FIG. 69 is a histogram showing expression of Homo sapiens breast cancer membrane protein 11 (BCMP11) T94936 transcripts which are detectable by amplicon as depicted in sequence name T94936 seg20 (SEQ ID NO: 432) in normal and cancerous ovary tissues.

FIG. 70 is a histogram showing expression of Homo sapiens breast cancer membrane protein 11 (BCMP11) T94936 transcripts which are detectable by amplicon as depicted in sequence name T94936 seg20 (SEQ ID NO: 432) in normal and cancerous breast tissues.

FIG. 71 is a histogram showing expression of Homo sapiens breast cancer membrane protein 11 (BCMP11) T94936 transcripts which are detectable by amplicon as depicted in sequence name T94936 seg20 (SEQ ID NO: 432) in different normal tissues.

FIG. 72 is a histogram showing Cancer and cell-line vs. normal tissue expression for Cluster HSTGFB1, demonstrating overexpression in epithelial malignant tumors, kidney malignant tumors, pancreas carcinoma and skin malignancies.

FIG. 73 is a histogram showing Expression of transforming growth factor, beta 1 (HSTGFB1) transcripts which are detectable by amplicon as depicted in sequence name HSTGFB1junc14-22-23 (SEQ ID NO: 474) in different normal tissues.

FIG. 74 is a histogram showing Expression of transforming growth factor, beta 1 (HSTGFB1) transcripts which are detectable by amplicon as depicted in sequence name HSTGFB1seg14-15 (SEQ ID NO: 471) in different normal tissues.

FIG. 75 is a histogram showing Expression of transforming growth factor, beta 1 (HSTGFB1) transcripts which are detectable by amplicon as depicted in sequence name HSTGFB1 seg7WT (SEQ ID NO:477) in different normal tissues.

FIG. 76 is a histogram showing expression of transforming growth factor, beta 1 transcripts which are detectable by HSTGFB1 seg 15, in normal and cancerous breast tissues.

FIG. 77 is a histogram showing Cancer and cell-line vs. normal tissue expression for Cluster S57296, demonstrating overexpression in a mixture of malignant tumors from different tissues, uterine malignancies, breast malignant tumors and epithelial malignant tumors.

FIG. 78 is a histogram showing Expression of Homo sapiens v-erb-b2 S57296 transcripts which are detectable by amplicon as depicted in sequence name S57296-B2Lnew seg58-59 (SEQ ID NO: 550) in normal and cancerous breast tissues.

FIG. 79 is a histogram showing expression of Homo sapiens v-erb-b2 S57296 transcripts which are detectable by amplicon as depicted in sequence name S57296 B2S seg-44 (SEQ ID NO: 552) in different normal tissues.

FIG. 80 is a histogram showing expression of Homo sapiens v-erb-b2 S57296 transcripts which are detectable by amplicon as depicted in sequence name S57296 B2Lnew seg58-59 (SEQ ID NO: 550) in different normal tissues.

FIG. 81 is a histogram showing expression of Homo sapiens v-erb-b2 S57296 transcripts which are detectable by amplicon as depicted in sequence name S57296WT seg63 (SEQ ID NO:497) in different normal tissues.

FIG. 82 is a histogram showing Expression of Homo sapiens v-erb-b2 S57296 transcripts which are detectable by amplicon as depicted in sequence name S57296WT seg63 (SEQ ID NO:497) in normal and cancerous breast tissues.

FIG. 83 is a histogram showing expression of Homo sapiens v-erb-b2 S57296 transcripts which are detectable by amplicon as depicted in sequence name S57296 B2S seg-44 (SEQ ID NO: 552) in normal and cancerous breast tissues.

FIG. 84 is a histogram showing combined expression of 4 sequences—S57296 B2S seg-44 (SEQ ID NO: 552), S57296 B2Lnew seg58-59 (SEQ ID NO: 550), HSSTROL seg20-21 (SEQ ID NO: 560), T94936 seg14 (SEQ ID NO: 563) in normal and cancerous breast tissues.

FIG. 85 is a histogram showing differential expression for cluster Z36249 in heart vs. other tissues.

FIG. 86 is a histogram showing expression of oligonucleotides in various tissues, including heart for cluster Z36249 using Affimetrix probe 206029_at.

FIG. 87 is a histogram showing expression of Homo sapiens ankyrin repeat domain 1 (cardiac muscle) Z36249 transcripts which are detectable by amplicon as depicted in sequence name Z36249 seg11-12 (SEQ ID NO:585) specifically in heart tissue.

FIG. 88 is a histogram showing Expression of Homo sapiens ankyrin repeat domain 1 (cardiac muscle) Z36249 transcripts which are detectable by amplicon as depicted in sequence name Z36249 seg14-16 (SEQ ID NO:588) specifically in heart tissue.

FIG. 89 is a histogram showing expression of Homo sapiens ankyrin repeat domain 1 (cardiac muscle) Z36249 transcripts which are detectable by amplicon as depicted in sequence name Z36249 junc23-25 (SEQ ID NO:591) specifically in heart tissue

FIG. 90 is a histogram showing Cancer and cell-line vs. normal tissue expression for Cluster M78530, demonstrating overexpression in ovarian carcinoma.

FIG. 91 is a histogram showing expression of Spondin 1 M78530 transcripts which are detectable by amplicon as depicted in sequence name M78530 seg37 (SEQ ID NO: 624) in normal and cancerous ovary tissues.

FIG. 92 is a histogram showing expression of Spondin 1 M78530 transcripts which are detectable by amplicon as depicted in sequence name M78530 seg40WT (SEQ ID NO: 627) in normal and cancerous ovary tissues.

FIG. 93 is a histogram showing Expression of spondin 1 transcripts which are detectable by junction of segments 2-4, in normal, benign and cancerous ovary tissues.

### DESCRIPTION OF PREFERRED EMBODIMENTS

The present invention provides variants, which may optionally be used as diagnostic markers.

Preferably these variants are useful as diagnostic markers for marker-detectable (also referred to herein as “variant-detectable”) diseases as described herein.

Differential variant markers are collectively described as “variant disease markers”.

The markers of the present invention, alone or in combination, can be used for prognosis, prediction, screening, early diagnosis, staging, therapy selection and treatment monitoring of a marker-detectable disease. For example, optionally and preferably, these markers may be used for staging the disease in patient (for example if the disease features cancer) and/or monitoring the progression of the disease. Furthermore, the markers of the present invention, alone or in combination, can be used for detection of the source of metastasis found in anatomical places other than the originating tissue, again in the example of cancer. Also, one or more of the markers may optionally be used in combination with one or more other disease markers (other than those described herein).

Biomolecular sequences (amino acid and/or nucleic acid sequences) uncovered using the methodology of the present invention and described herein can be efficiently utilized as tissue or pathological markers and/or as drugs or drug targets for treating or preventing a disease.

These markers are specifically released to the bloodstream under conditions of a particular disease, and/or are otherwise expressed at a much higher level and/or specifically expressed in tissue or cells afflicted with or demonstrating the disease. The measurement of these markers, alone or in combination, in patient samples provides information that the diagnostician can correlate with a probable diagnosis of a particular disease and/or a condition that is indicative of a higher risk for a particular disease.

The present invention therefore also relates to diagnostic assays for marker-detectable disease and/or an indicative condition, and methods of use of such markers for detection of marker-detectable disease and/or an indicative condition, optionally and preferably in a sample taken from a subject (patient), which is more preferably some type of blood sample.

Information given in the text with regard to cellular localization was determined according to four different software programs: (i) tmhmm (from Center for Biological Sequence Analysis, Technical University of Denmark DTU, http://www.cbs.dtu.dk/services/TMHMM/TMHMM2.0b.guide.php) or (ii) tmpred (from EMBnet, maintained by the ISREC Bionformatics group and the LICR Information Technology Office, Ludwig Institute for Cancer Research, Swiss Institute of Bioinformatics, http://www.ch.embnet.org/software/TMPRED_form.html) for transmembrane region prediction; (iii) signalp_hmm or (iv) signalp_nn (both from Center for Biological Sequence Analysis, Technical University of Denmark DTU, http://www.cbs.dtu.dk/services/SignalP/background/prediction.php) for signal peptide prediction. The terms “signalp_hmm” and “signalp_nn” refer to two modes of operation for the program SignalP: hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform [Einat Hazkani-Covo, Erez Levanon, Galit Rotman, Dan Graur and Amit Novik; (2004) “Evolution of multicellularity in metazoa: comparative analysis of the subcellular localization of proteins in Saccharomyces, Drosophila and Caenorhabditis.” Cell Biology International 2004; 28(3):171-8.], which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

Information is given in the text with regard to SNPs (single nucleotide polymorphisms). A description of the abbreviations is as follows. “T->C”, for example, means that the SNP results in a change at the position given in the table from T to C. Similarly, “M->Q”, for example, means that the SNP has caused a change in the corresponding amino acid sequence, from methionine (M) to glutamine (Q). If, in place of a letter at the right hand side for the nucleotide sequence SNP, there is a space, it indicates that a frameshift has occurred. A frameshift may also be indicated with a hyphen (-). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows construction of links directly from position-specific annotation in the feature table to specialized protein-related databases. The FTId is always the last component of a feature in the description field, as follows: FTId=XXX_number, in which XXX is the 3-letter code for the specific feature key, separated by an underscore from a 6-digit number. In the table of the amino acid mutations of the wild type proteins of the selected splice variants of the invention, the header of the first column is “SNP position(s) on amino acid sequence”, representing a position of a known mutation on amino acid sequence. SNPs may optionally be used as diagnostic markers according to the present invention, alone or in combination with one or more other SNPs and/or any other diagnostic marker. Preferred embodiments of the present invention comprise such SNPs, including but not limited to novel SNPs on the known (WT or wild type) protein sequences given below, as well as novel nucleic acid and/or amino acid sequences formed through such SNPs, and/or any SNP on a variant amino acid and/or nucleic acid sequence described herein.

Information given in the text with regard to the Homology to the known proteins was determined by Smith-Waterman version 5.1.2 using special (non default) parameters as follows:

model=sw.model

GAPEXT=0

GAPOP=100.0
MATRIX=blosum100

Information is given with regard to overexpression of a cluster in cancer based on ESTs. A key to the p values with regard to the analysis of such overexpression is as follows:
library-based statistics: P-value without including the level of expression in cell-lines (P1)
library based statistics: P-value including the level of expression in cell-lines (P2)
EST clone statistics: P-value without including the level of expression in cell-lines (SP1)
EST clone statistics: predicted overexpression ratio without including the level of expression in cell-lines (R3)
EST clone statistics: P-value including the level of expression in cell-lines (SP2)
EST clone statistics: predicted overexpression ratio including the level of expression in cell-lines (R4)

Library-based statistics refer to statistics over an entire library, while EST clone statistics refer to expression only for ESTs from a particular tissue or cancer.

Information is given with regard to overexpression of a cluster in cancer based on microarrays. As a microarray reference, in the specific segment paragraphs, the unabbreviated tissue name was used as the reference to the type of chip for which expression was measured. There are two types of microarray results: those from microarrays prepared according to a design by the present inventors, for which the microarray fabrication procedure is described in detail in Materials and Experimental Procedures section herein; and those results from microarrays using Affymetrix technology. As a microarray reference, in the specific segment paragraphs, the unabbreviated tissue name was used as the reference to the type of chip for which expression was measured. For microarrays prepared according to a design by the present inventors, the probe name begins with the name of the cluster (gene), followed by an identifying number. Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgu133.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgu133plus.affx). The probe names follow the Affymetrix naming convention. The data is available from NCBI Gene Expression Omnibus (see www.ncbi.nlm.nih.gov/projects/geo/ and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 Apr. 9).

Oligonucleotide probes for use with arrays designed by the present inventors:

>S67314_0_0_741 (SEQ ID NO:17)

(SEQ ID NO:685)

CACAGAGCCAGGATGTTCTTCTGACCTCAGTATCTACTCCAGCTCCAGCT
>S67314_0_0_744 (SEQ ID NO:18)

(SEQ ID NO:686)

TGGCATGCTGGAACATGGACTCTAGCTAGCAAGAAGGGCTCAAGGAGGTG
>HSPROSAP_0_0_11823 (SEQ ID NO:19)

(SEQ ID NO:687)

CCTCTGGGGTAGGTTACTATCCTCTTTGTCCTGCCAGTACCCCTAGAAAT
>HSPROSAP_0_9_0 (SEQ ID NO:20)

(SEQ ID NO:688)

TTGGTGTTTCGGCATGGAGACCGAAGTCCCATTGACACCTTTCCCACTGA
>D11581_0_0_2570 (SEQ ID NO:21)

(SEQ ID NO:689)

ATGAGGGGAGATTGCCTTCCACTACACATAAGTATGGTCAAGTATGAAAT
>HSMUC1A_0_37_0 (SEQ ID NO:22)

(SEQ ID NO:690)

AAAAGGAGACTTCGGCTACCCAGAGAAGTTCAGTGCCCAGCTCTACTGAG
>HSMUC1A_0_0_11364 (SEQ ID NO:23)

(SEQ ID NO:691)

AAAGGCTGGCATAGGGGGAGGTTTCCCAGGTAGAAGAAGAAGTGTCAGCA
>HSMUC1A_0_0_11365 (SEQ ID NO:24)

(SEQ ID NO:692)

AATTAACCCTTTGAGAGCTGGCCAGGACTCTGGACTGATTACCCCAGCCT
>HSAPHOL_0_11_0 (SEQ ID NO:25)

(SEQ ID NO:25)

GGAACATTCTGGATCTGACCCTCCCAGTCTCATCTCCTGACCCTCCCACT
>HSCREACT_0_31_0 (SEQ ID NO:26)

(SEQ ID NO:630)

CCTCCCCTTTTCCACACGAACCTTGTGGGGCTGTGAATTCTTTCTTCATC
>HUMGRP5E_0_0_16630 (SEQ ID NO:10)
GCTGATATGGAAGTTGGGGAATCTGAATTGCCAGAGAATCTTGGGAAGAG
>HUMGRP5E_0_2_0 (SEQ ID NO:11)
TCTCATAGAAGCAAAGGAGAACAGAAACCACCAGCCACCTCAACCCAAGG
>M78530_0_6_0 (SEQ ID NO:12)
CTTCCTACACACATCTAGACGTTCAAGTTTGCAAATCAGTTTTTAGCAAG

In the heart specific clusters, a first set of abbreviations is used for the first histogram

ADP=adipocyte
BLD=blood
BLDR=bladder
BRN=brain
BONE=bone
BM=bone marrow
BRS=mammary gland
CAR=cartilage
CNS=central nervous system
COL=colon
E-ADR=endocrine_adrenal_gland
E-PAN=endocrine_pancreas
E-PT=endocrine_parathyroid_thyroid
ENDO=endocrine_unchar
EPID=epididymis
GI=gastrointestinal tract
GU=genitourinary
HN=head and neck
HRT=heart
KD=kidney
LI=liver
LUNG=lung
LN=lymph node
MUS=muscle
OV=ovary
PNS=peripheral nervous system
PRO=prostate
SKIN=skin
SPL=spleen
SYN=synovial membrane
TCELL=immune T cells
THYM=thymus
TST=testes
UTER=cervix-uterus
VAS=vascular

In the second histogram(s) of the heart paragraph, the oligo-probe names are abbreviated/enumerated as follows:

| | |
|---|---|
| “adipocyte”, | “A1”; |
| “adrenalcortex”, | “A2”; |
| “adrenalgland”, | “A3”; |
| “amygdala”, | “A4”; |
| “appendix”, | “A5”; |
| “atrioventricularnode”, | “A6”; |
| “bm_cd105_endothelial”, | “E1”; |
| “bm_cd33_myeloid”, | “M1”; |
| “bm_cd34_”, | “B1”; |
| “bm_cd71_earlyerythroid”, | “E1”; |
| “bonemarrow”, | “B2”; |
| “bronchialepithelialcells”, | “B3”; |
| “cardiacmyocytes”, | “C1”; |
| “caudatenucleus”, | “C2”; |
| “cerebellum”, | “C3”; |
| “cerebellumpeduncles”, | “C4”; |
| “ciliaryganglion”, | “C5”; |
| “cingulatecortex”, | “C6”; |
| “globuspallidus”, | “G1”; |
| “heart”, | “H1”; |
| “hypothalamus”, | “H2”; |
| “kidney”, | “K1”; |
| “liver”, | “L1”; |
| “lung”, | “L2”; |
| “lymphnode”, | “L3”; |
| “medullaoblongata”, | “M1”; |
| “occipitallobe”, | “O1”; |
| “olfactorybulb”, | “O2”; |
| “ovary”, | “O3”; |
| “pancreas”, | “P1”; |
| “pancreaticislets”, | “P2”; |
| “parietallobe”, | “P3”; |
| “pb_bdca4_dentritic_cells”, | “P4”; |
| “pb_cd14_monocytes”, | “P5”; |
| “pb_cd19_bcells”, | “P6”; |
| “pb_cd4_tcells”, | “P7”; |
| “pb_cd56_nkcells”, | “P8”; |
| “pb_cd8_tcells”, | “P9”; |
| “pituitary”, | “Pa”; |
| “placenta”, | “Pb”; |
| “pons”, | “Pc”; |
| “prefrontalcortex”, | “Pd”; |
| “prostate”, | “Pe”; |
| “salivarygland”, | “S1”; |
| “skeletalmuscle”, | “S2”; |
| “skin”, | “S3”; |
| “smoothmuscle”, | “S4”; |
| “spinalcord”, | “S5”; |
| “subthalamicnucleus”, | “S6”; |
| “superiorcervicalganglion”, | “S7”; |
| “temporallobe”, | “T1”; |
| “testis”, | “T2”; |
| “testisgermcell”, | “T3”; |
| “testisinterstitial”, | “T4”; |
| “testisleydigcell”, | “T5”; |
| “testisseminiferoustubule”, | “S6”; |
| “thalamus”, | “T7”; |
| “thymus”, | “T8”; |
| “thyroid”, | “T9”; |
| “tonsil”, | “Ta”; |
| “trachea”, | “Tb”; |
| “trigeminalganglion”, | “Tc”; |
| “uterus”, | “U1”; |
| “uteruscorpus”, | “U2”; |
| “wholeblood”, | “W1”; |
| “wholebrain”, | “W2”; |

It should be noted that the terms “segment”, “seg” and “node” are used interchangeably in reference to nucleic acid sequences of the present invention, they refer to portions of nucleic acid sequences that were shown to have one or more properties as described below. They are also the building blocks that were used to construct complete nucleic acid sequences as described in greater detail below. Optionally and preferably, they are examples of oligonucleotides which are embodiments of the present invention, for example as amplicons, hybridization units and/or from which primers and/or complementary oligonucleotides may optionally be derived, and/or for any other use.

As used herein the phrase “disease” includes any type of pathology and/or damage, including both chronic and acute damage, as well as a progress from acute to chronic damage.

The term “marker” in the context of the present invention refers to a nucleic acid fragment, a peptide, or a polypeptide, which is differentially present in a sample taken from patients (subjects) having one of the herein-described diseases or conditions, as compared to a comparable sample taken from subjects who do not have one the above-described diseases or conditions.

The phrase “differentially present” refers to differences in the quantity of a marker present in a sample taken from patients having one of the herein-described diseases or conditions as compared to a comparable sample taken from patients who do not have one of the herein-described diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present. Optionally, a relatively low amount of up-regulation may serve as the marker, as described herein. One of ordinary skill in the art could easily determine such relative levels of the markers; further guidance is provided in the description of each individual marker below.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

As used herein, the term “level” refers to expression levels of RNA and/or protein or to DNA copy number of a marker of the present invention.

Typically the level of the marker in a biological sample obtained from the subject is different (i.e., increased or decreased) from the level of the same variant in a similar sample obtained from a healthy individual (examples of biological samples are described herein).

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

A “test amount” of a marker refers to an amount of a marker in a subject's sample that is consistent with a diagnosis of a particular disease or condition. A test amount can be either in absolute amount (e.g., microgram/ml) or a relative amount (e.g., relative intensity of signals).

A “control amount” of a marker can be any amount or a range of amounts to be compared against a test amount of a marker. For example, a control amount of a marker can be the amount of a marker in a patient with a particular disease or condition or a person without such a disease or condition. A control amount can be either in absolute amount (e.g., microgram/ml) or a relative amount (e.g., relative intensity of signals).

“Detect” refers to identifying the presence, absence or amount of the object to be detected.

A “label” includes any moiety or item detectable by spectroscopic, photo chemical, biochemical, immunochemical, or chemical means. For example, useful labels include ³²P, ³⁵S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide (or other epitope), refers to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

In another embodiment, the present invention relates to bridges, tails, heads and/or insertions, and/or analogs, homologs and derivatives of such peptides. Such bridges, tails, heads and/or insertions are described in greater detail below with regard to the Examples.

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

As used herein a “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

As used herein “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

Optionally and preferably, a bridge between a tail or a head or a unique insertion, and a “known protein” portion of a variant, comprises at least about 10 amino acids, more preferably at least about 20 amino acids, most preferably at least about 30 amino acids, and even more preferably at least about 40 amino acids, in which at least one amino acid is from the tail/head/insertion and at least one amino acid is from the “known protein” portion of a variant. Also optionally, the bridge may comprise any number of amino acids from about 10 to about 40 amino acids (for example, 10, 11, 12, 13 . . . 37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME_P1): a sequence starting from any of amino acid numbers 49−x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequence, so it should be read such that 49−x (for example) is not less than 1, nor 50+((n−2)−x) (for example) greater than the total sequence length.

In another embodiment, this invention provides antibodies specifically recognizing the splice variants and polypeptide fragments thereof of this invention. Preferably such antibodies differentially recognize splice variants of the present invention but do not recognize a corresponding known protein (such known proteins are discussed with regard to their splice variants in the Examples below).

In another embodiment, this invention provides an isolated nucleic acid molecule encoding for a splice variant according to the present invention, having a nucleotide sequence as set forth in any one of the sequences listed herein, or a sequence complementary thereto. In another embodiment, this invention provides an isolated nucleic acid molecule, having a nucleotide sequence as set forth in any one of the sequences listed herein, or a sequence complementary thereto. In another embodiment, this invention provides an oligonucleotide of at least about 12 nucleotides, specifically hybridizable with the nucleic acid molecules of this invention. In another embodiment, this invention provides vectors, cells, liposomes and compositions comprising the isolated nucleic acids of this invention.

In another embodiment, this invention provides a method for detecting a splice variant according to the present invention in a biological sample, comprising: contacting a biological sample with an antibody specifically recognizing a splice variant according to the present invention under conditions whereby the antibody specifically interacts with the splice variant in the biological sample but do not recognize known corresponding proteins (wherein the known protein is discussed with regard to its splice variant(s) in the Examples below), and detecting said interaction; wherein the presence of an interaction correlates with the presence of a splice variant in the biological sample.

In another embodiment, this invention provides a method for detecting a splice variant nucleic acid sequences in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a splice variant nucleic acid sequence in the biological sample.

According to the present invention, the splice variants described herein are non-limiting examples of markers for diagnosing marker-detectable disease and/or an indicative condition. Each splice variant marker of the present invention can be used alone or in combination, for various uses, including but not limited to, prognosis, prediction, screening, early diagnosis, determination of progression, therapy selection and treatment monitoring of marker-detectable disease and/or an indicative condition, including a transition from an indicative condition to marker-detectable disease.

According to optional but preferred embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

Panels of Markers According to the Present Invention Optionally with One or More Known Marker(s)

The present invention is of methods, uses, devices and assays for diagnosis of a disease or condition. Optionally a plurality of biomarkers (or markers) may be used with the present invention. The plurality of markers may optionally include a plurality of markers described herein, and/or one or more known markers. The plurality of markers is preferably then correlated with the disease or condition. For example, such correlating may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlate with the disease or condition.

Alternatively, such correlating may optionally comprise determining the concentration of each of the plurality of markers, calculating a single index value based on the concentration of each of the plurality of markers, and comparing the index value to a threshold level.

Also alternatively, such correlating may optionally comprise determining a temporal change in at least one of the markers, and wherein the temporal change is used in the correlating step.

Also alternatively, such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Also alternatively, such correlating may optionally comprise determining whether a ratio of marker concentrations for two markers is outside a range and/or above or below a threshold. Optionally, if the ratio is above or below the threshold level and/or outside a range, the ratio correlates with the disease or condition.

Optionally, a combination of two or more these correlations may be used with a single panel and/or for correlating between a plurality of panels.

Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers are outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. For example, with stroke as a non-limiting example of a disease or condition, certain markers in a panel may be commonly used to diagnose the existence of a stroke, while other members of the panel may indicate if an acute stroke has occurred, while still other members of the panel may indicate if a non-acute stroke has occurred. Markers may also be commonly used for multiple purposes by, for example, applying a different threshold or a different weighting factor to the marker for the different purpose(s). For example, again with stroke as a non-limiting example of a disease or condition, a marker at one concentration or weighting may be used, alone or as part of a larger panel, to indicate if an acute stroke has occurred, and the same marker at a different concentration or weighting may be used, alone or as part of a larger panel, to indicate if a non-acute stroke has occurred.

Preferred panels comprise markers for the following purposes: diagnosis of a disease; diagnosis of disease and indication if the disease is in an acute phase and/or if an acute attack of the disease has occurred (for example for CVS, heart disease, stroke and/or cerebrovascular accident); diagnosis of disease and indication if the disease is in a non-acute phase and/or if a non-acute attack of the disease has occurred (for example for CVS, heart disease, stroke and/or cerebrovascular accident); indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those diseases that may feature one or more similar or identical symptoms.

In certain embodiments, one or more diagnostic or prognostic indicators are correlated to a condition or disease by merely the presence or absence of the indicator(s). In other embodiments, threshold level(s) of a diagnostic or prognostic indicator(s) can be established, and the level of the indicator(s) in a patient sample can simply be compared to the threshold level(s). The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art (see for example Hanley et al., Radiology 143: 29-36 (1982), incorporated by reference as if fully set forth herein).

One or more markers may lack diagnostic or prognostic value when considered alone, but when used as part of a panel, such markers may be of great value in determining a particular diagnosis/prognosis. In preferred embodiments, particular thresholds for one or more markers in a panel are not relied upon to determine if a profile of marker levels obtained from a subject are indicative of a particular diagnosis/prognosis. Rather, the present invention may utilize an evaluation of the entire marker profile by plotting ROC curves for the sensitivity of a particular panel of markers versus 1-(specificity) for the panel at various cutoffs. In these methods, a profile of marker measurements from a subject is considered together to provide a global probability (expressed either as a numeric score or as a percentage risk) that an individual has had a disease, is at risk for developing such a disease, optionally the type of disease which the individual has had or is at risk for, and so forth etc. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In preferred embodiments, markers and/or marker panels are selected to exhibit at least 70% sensitivity, more preferably at least 80% sensitivity, even more preferably at least 85% sensitivity, still more preferably at least 90% sensitivity, and most preferably at least 95% sensitivity, combined with at least 70% specificity, more preferably at least 80% specificity, even more preferably at least 85% specificity, still more preferably at least 90% specificity, and most preferably at least 95% specificity. In particularly preferred embodiments, both the sensitivity and specificity are at least 75%, more preferably at least 80%, even more preferably at least 85%, still more preferably at least 90%, and most preferably at least 95%. Sensitivity and/or specificity may optionally be determined as described above, with regard to the construction of ROC graphs and so forth, for example.

According to preferred embodiments of the present invention, individual markers and/or combinations (panels) of markers may optionally be used for diagnosis of time of onset of a disease or condition. Such diagnosis may optionally be useful for a wide variety of conditions, preferably including those conditions with an abrupt onset.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. For example, in individuals not exhibiting the condition, the chance of a given outcome may be about 3%. In preferred embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

The skilled artisan will understand that associating a prognostic indicator with a predisposition to an adverse outcome is a statistical analysis. For example, a marker level of greater than 80 pg/mL may signal that a patient is more likely to suffer from an adverse outcome than patients with a level less than or equal to 80 pg/mL, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a prognostic indicator with a predisposition to an adverse outcome are described hereinafter.

In other embodiments, a threshold degree of change in the level of a prognostic or diagnostic indicator can be established, and the degree of change in the level of the indicator in a patient sample can simply be compared to the threshold degree of change in the level. A preferred threshold change in the level for markers of the invention is about 5%, about 10%, about 15%, about 20%, about 25%, about 30%, about 50%, about 75%, about 100%, and about 150%. The term “about” in this context refers to +/−10%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

Exemplary, non-limiting methods and systems for identification of suitable biomarkers for marker panels are now described. Methods and systems for the identification of a one or more markers for the diagnosis, and in particular for the differential diagnosis, of disease have been described previously. Suitable methods for identifying markers useful for the diagnosis of disease states are described in detail in U.S. patent application no. 2004-0126767, entitled METHOD AND SYSTEM FOR DISEASE DETECTION USING MARKER COMBINATIONS, filed Dec. 27, 2002, hereby incorporated by reference in its entirety as if fully set forth herein. One skilled in the art will also recognize that univariate analysis of markers can be performed and the data from the univariate analyses of multiple markers can be combined to form panels of markers to differentiate different disease conditions.

In developing a panel of markers useful in diagnosis, data for a number of potential markers may be obtained from a group of subjects by testing for the presence or level of certain markers. The group of subjects is divided into two sets, and preferably the first set and the second set each have an approximately equal number of subjects. The first set includes subjects who have been confirmed as having a disease or, more generally, being in a first condition state. For example, this first set of patients may be those that have recently had a disease and/or a particular type of the disease. The confirmation of this condition state may be made through more rigorous and/or expensive testing, preferably according to a previously defined diagnostic standard. Hereinafter, subjects in this first set will be referred to as “diseased”.

The second set of subjects are simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects.

The data obtained from subjects in these sets includes levels of a plurality of markers. Preferably, data for the same set of markers is available for each patient. This set of markers may include all candidate markers which may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve as described above.

As discussed above, the measurement of the level of a single marker may have limited usefulness. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. In order to develop lower-cost panels which require the measurement of fewer marker levels, certain markers may be eliminated from the panel. In this regard, the effective contribution of each marker in the panel may be determined to identify the relative importance of the markers. In one embodiment, the weighting coefficients resulting from the optimization process may be used to determine the relative importance of each marker. The markers with the lowest coefficients may be eliminated.

Individual panel response values may also be used as markers in the methods described herein. For example, a panel may be constructed from a plurality of markers, and each marker of the panel may be described by a function and a weighting factor to be applied to that marker (as determined by the methods described above). Each individual marker level is determined for a sample to be tested, and that level is applied to the predetermined function and weighting factor for that particular marker to arrive at a sample value for that marker. The sample values for each marker are added together to arrive at the panel response for that particular sample to be tested. For a “diseased” and “non-diseased” group of patients, the resulting panel responses may be treated as if they were just levels of another disease marker.

Measures of test accuracy may be obtained as described in Fischer et al., Intensive Care Med. 29: 1043-51, 2003 (hereby incorporated by reference as if fully set forth herein), and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures: at least 75% sensitivity, combined with at least 75% specificity; ROC curve area of at least 0.7, more preferably at least 0.8, even more preferably at least 0.9, and most preferably at least 0.95; and/or a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

According to other preferred embodiments of the present invention, a splice variant protein or a fragment thereof, or a splice variant nucleic acid sequence or a fragment thereof, may be featured as a biomarker for detecting marker-detectable disease and/or an indicative condition, such that a biomarker may optionally comprise any of the above.

According to still other preferred embodiments, the present invention optionally and preferably encompasses any amino acid sequence or fragment thereof encoded by a nucleic acid sequence corresponding to a splice variant protein as described herein. Any oligopeptide or peptide relating to such an amino acid sequence or fragment thereof may optionally also (additionally or alternatively) be used as a biomarker, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

Non-limiting examples of methods or assays are described below.

The present invention also relates to kits based upon such diagnostic methods or assays.

### Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific, lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the trasgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

### Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, ³²P, and ³⁵S, Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include ³H, ¹⁴C, ³²P, and ³⁵S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

### NAT Assays

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. NatI. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes: see for example Segev, PCT Publication No. W09001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station. describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for a disease and/or pathological condition both rapidly and easily.

### Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Preferably, nucleic acid sequence homology/identity is determined by using BlastN software of the National Center of Biotechnology Information (NCBI) using default parameters, which preferably include using the DUST filter program, and also preferably include having an E value of 10, filtering low complexity sequences and a word size of 11. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Table 1 specifies non-conventional or modified amino acid which can be used with the present invention.

TABLE 1

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| Carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| Carboxylate | | L-N-methylglutamic acid | Nmglu |
| Cyclohexylalanine | Chexa | L-N-methylhistidine | Nmhis |
| Cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

Since the peptides of the present invention are preferably utilized in diagnostics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

### Antibodies:

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Monoclonal antibody development may optionally be performed according to any method that is known in the art. The method described below is provided for the purposes of description only and is not meant to be limiting in any way.

Step 1: Immunization of Mice and Selection of Mouse Donors for Generation of Hybridoma Cells:

Producing mAb requires immunizing an animal, usually a mouse, by injection of an antigen X to stimulate the production of antibodies targeted against X. Antigen X can be the whole protein or any sequence thereof that gives rise to a determinant. According to the present invention, optionally and preferably such antigens may include but are not limited to any variant described herein or a portion thereof, including but not limited to any head, tail, bridge or unique insertion, or a bridge to such head, tail or unique insertion, or any other epitope described herein according to the present invention. Injection of peptides requires peptide design (with respect to protein homology, antigenicity, hydrophilicity, and synthetic suitability) and synthesis. The antigen is optionally and preferably prepared for injection either by emulsifying the antigen with Freund's adjuvant or other adjuvants or by homogenizing a gel slice that contains the antigen. Intact cells, whole membranes, and microorganisms are sometimes optionally used as immunogens. Other immunogens or adjuvants may also optionally be used.

In general, mice are immunized every 2-3 weeks but the immunization protocols are heterogeneous. When a sufficient antibody titer is reached in serum, immunized mice are euthanized and the spleen removed to use as a source of cells for fusion with myeloma cells.

Step 2: Screening of Mice for Antibody Production

After several weeks of immunization, blood samples are optionally and preferably obtained from mice for measurement of serum antibodies. Several techniques have been developed for collection of small volumes of blood from mice (Loeb and Quimby 1999). Serum antibody titer is determined with various techniques, such as enzyme-linked immunosorbent assay (ELISA) and flow cytometry, and/or immunoassays for example (for example a Western blot may optionally be used). If the antibody titer is high, cell fusion can optionally be performed. If the titer is too low, mice can optionally be boosted until an adequate response is achieved, as determined by repeated blood sampling. When the antibody titer is high enough, mice are commonly boosted by injecting antigen without adjuvant intraperitoneally or intravenously (via the tail veins) 3 days before fusion but 2 weeks after the previous immunization. Then the mice are euthanized and their spleens removed for in vitro hybridoma cell production.

Step 3: Preparation of Myeloma Cells

Fusing antibody-producing spleen cells, which have a limited life span, with cells derived from an immortal tumor of lymphocytes (myeloma) results in a hybridoma that is capable of unlimited growth. Myeloma cells are immortalized cells that are optionally and preferably cultured with 8-azaguanine to ensure their sensitivity to the hypoxanthine-aminopterin-thymidine (HAT) selection medium used after cell fusion. The selection growth medium contains the inhibitor aminopterin, which blocks synthetic pathways by which nucleotides are made. Therefore, the cells must use a bypass pathway to synthesize nucleic acids, a pathway that is defective in the myeloma cell line to which the normal antibody-producing cells are fused. Because neither the myeloma nor the antibody-producing cell will grow on its own, only hybrid cells grow. The HAT medium allows only the fused cells to survive in culture. A week before cell fusion, myeloma cells are grown in 8-azaguanine. Cells must have high viability and rapid growth.

The antibody forming cells are isolated from the mouse's spleen and are then fused with a cancer cell (such as cells from a myeloma) to make them immortal, which means that they will grow and divide indefinitely. The resulting cell is called a hybridoma.

Step 4: Fusion of Myeloma Cells with Immune Spleen Cells and antibody screening

Single spleen cells from the immunized mouse are fused with the previously prepared myeloma cells. Fusion is accomplished by co-centrifuging freshly harvested spleen cells and myeloma cells in polyethylene glycol, a substance that causes cell membranes to fuse. Alternatively, the cells are centrifuged, the supernatant is discarded and PEG is then added. The cells are then distributed to 96 well plates containing feeder cells derived from saline peritoneal washes of mice. Feeder cells are believed to supply growth factors that promote growth of the hybridoma cells (Quinlan and Kennedy 1994). Commercial preparations that result from the collection of media supporting the growth of cultured cells and contain growth factors are available that can be used in lieu of mouse-derived feeder cells. It is also possible to use murine bone marrow-derived macrophages as feeder cells (Hoffman and others 1996).

Once hybridoma colonies reach a satisfactory cell count, the plates are assayed by an assay, eg ELISA or a regular immunoassay such as RIA for example, to determine which colonies are secreting antibodies to the immunogen. Cells from positive wells are isolated and expanded. Conditioned medium from each colony is retested to verify the stability of the hybridomas (that is, they continue to produce antibody).

Step 5: Cloning of Hybridoma Cell Lines by “Limiting Dilution” or Expansion and Stabilization of Clones by Ascites Production

At this step new, small clusters of hybridoma cells from the 96 well plates can be grown in tissue culture followed by selection for antigen binding or grown by the mouse ascites method with cloning at a later time.

For prolonged stability of the antibody-producing cell lines, it is necessary to clone and then recline the chosen cells. Cloning consists of subcloning the cells by either limiting dilution at an average of less than one cell in each culture well or by platingout the cells in a thin layer of semisolid agar of methyl cellulose or by single-cell manipulation. At each stage, cultures are assayed for production of the appropriate antibodies.

Step 6: Antibody Purification

The secreted antibodies are optionally purified, preferably by one or more column chromatography steps and/or some other purification method, including but not limited to ion exchange, affinity, hydrophobic interaction, and gel permeation chromatography. The operation of the individual chromatography step, their number and their sequence is generally tailored to the specific antibody and the specific application.

Large-scale antibody production may also optionally and preferably be performed according to the present invention. Two non-limiting, illustrative exemplary methods are described below for the purposes of description only and are not meant to be limiting in any way.

In vivo production may optionally be performed with ascites fluid in mice. According to this method, hybridoma cell lines are injected into the peritoneal cavity of mice to produce ascitic fluid (ascites) in its abdomen; this fluid contains a high concentration of antibody.

An exemplary in vitro method involves the use of culture flasks. In this method, monoclonal antibodies can optionally be produced from the hybridoma using gas permeable bags or cell culture flasks.

### Antibody Engineering in Phage Display Libraries:

PCT Application No. WO 94/18219, and its many US equivalents, including U.S. Pat. No. 6,096,551, all of which are hereby incorporated by reference as if fully set forth herein, describes methods for producing antibody libraries using universal or randomized immunoglobulin light chains, by using phage display libraries. The method involves inducing mutagenesis in a complementarity determining region (CDR) of an immunoglobulin light chain gene for the purpose of producing light chain gene libraries for use in combination with heavy chain genes and gene libraries to produce antibody libraries of diverse and novel immunospecificities. The method comprises amplifying a CDR portion of an immunoglobulin light chain gene by polymerase chain reaction (PCR) using a PCR primer oligonucleotide. The resultant gene portions are inserted into phagemids for production of a phage display library, wherein the engineered light chains are displayed by the phages, for example for testing their binding specificity.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. A scFv antibody fragment is an engineered antibody derivative that includes heavy- and light chain variable regions joined by a peptide linker. The minimal size of antibody molecules are those that still comprise the complete antigen binding site. ScFv antibody fragments are potentially more effective than unmodified IgG antibodies. The reduced size of 27-30 kDa permits them to penetrate tissues and solid tumors more readily. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)]. Optionally, there may be 1, 2 or 3 CDRs of different chains, but preferably there are 3 CDRs of 1 chain. The chain could be the heavy or the light chain.

Humanized forms of non-human (e.g., murine) antibodies are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is nonhuman. These nonhuman amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

### Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead.

Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Preferably used are antibodies which specifically interact with the polypeptides of the present invention and not with wild type proteins or other isoforms thereof, for example. Such antibodies are directed, for example, to the unique sequence portions of the polypeptide variants of the present invention, including but not limited to bridges, heads, tails and insertions described in greater detail below. Preferred embodiments of antibodies according to the present invention are described in greater detail with regard to the section entitled “Antibodies”.

Radio-immunoassay (RIA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a calorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

### Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

### Display Libraries

According to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel L B et al. “Screening of cyclic peptide phage libraries identifies ligands that bind streptavidin with high affinities” Biochemistry 1995 Nov. 28; 34(47):15430-5; Davies E L et al., “Selection of specific phage-display antibodies using libraries derived from chicken immunoglobulin genes” J Immunol Methods 1995 Oct. 12; 186(1):125-35; Jones C RT al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci USA 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

### Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest and Herceptin (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

### Surrogate Markers:

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, nucleic acid tests and imaging modalities).

Surrogate endpoints were used first mainly in the cardiovascular area. For example, antihypertensive drugs have been approved based on their effectiveness in lowering blood pressure. Similarly, in the past, cholesterol-lowering agents have been approved based on their ability to decrease serum cholesterol, not on the direct evidence that they decrease mortality from atherosclerotic heart disease. The measurement of cholesterol levels is now an accepted surrogate marker of atherosclerosis. In addition, currently two commonly used surrogate markers in HIV studies are CD4+ T cell counts and quantitative plasma HIV RNA (viral load).

### Monoclonal Antibody Therapy:

Monoclonal antibodies by identifying and binding to the target cells alert other cells in the immune system to the presence of the cancer cells. Monoclonal antibody therapy is a form of passive immunotherapy because the antibodies are made in large quantities outside the body (in the lab) rather than by a person's immune system.

Two types of monoclonal antibodies are used in cancer treatments:

1. Naked monoclonal antibodies.

2. Conjugated monoclonal antibodies—joined to a chemotherapy drug, radioactive particle, or a toxin (a substance that poisons cells).

1. Naked Monoclonal Antibodies:

Naked antibodies attach themselves to specific antigens on cancer cells. They can act in different ways: some mark the cancer cell for the immune system to destroy it, while others attach to receptors and block their ligand binding site and may therefore prevent the cancer cells from growing rapidly. Trastuzumab (Herceptin), a naked MAb used against advanced breast cancer, works in that way.

2. Conjugated Monoclonal Antibodies:

Conjugated monoclonal antibodies are joined to drugs, toxins, or radioactive atoms. They are used as delivery vehicles to take those substances directly to the cancer cells. The MAb acts as a homing device, circulating in the body until it finds a cancer cell with a matching antigen. It delivers the toxic substance to where it is needed most, minimizing damage to normal cells in other parts of the body. Conjugated MAbs are also sometimes referred to as “tagged,” “labeled,” or “loaded” antibodies. MAbs with chemotherapy drugs attached are generally referred to as chemolabeled. MAbs with radioactive particles attached are referred to as radiolabeled, and this type of therapy is known as radioimmunotherapy (RIT). MAbs attached to toxins are called immunotoxins.

An illustrative, non-limiting example is provided herein of a method of treatment of a patient with an antibody to a variant as described herein, such that the variant is a target of the antibody. A patient with breast cancer is treated with a radiolabeled humanized antibody against an appropriate breast cancer target as described herein. The patient is optionally treated with a dosage of labeled antibody ranging from 10 to 30 mCi. Of course any type of therapeutic label may optionally be used.

The following sections relate to Candidate Marker Examples. It should be noted that Table numbering is restarted within each Example, which starts with the words “Description for Cluster”.

### Candidate Marker Examples Section

This Section relates to Examples of sequences according to the present invention, including illustrative methods of selection thereof with regard to cancer; other markers were selected as described below for the individual markers.

Description of the Methodology Undertaken to Uncover the Biomolecular Sequences of the Present Invention

Human ESTs and cDNAs were obtained from GenBank versions 136 (Jun. 15, 2003 ftp.ncbi.nih.gov/genbank/release.notes/gb136.release.notes); NCBI genome assembly of April 2003; RefSeq sequences from June 2003; Genbank version 139 (December 2003); Human Genome from NCBI (Build 34) (from October 2003); and RefSeq sequences from December 2003. With regard to GenBank sequences, the human EST sequences from the EST (GBEST) section and the human mRNA sequences from the primate (GBPRI) section were used; also the human nucleotide RefSeq mRNA sequences were used (see for example www.ncbi.nlm.nih.gov/Genbank/GenbankOverview.html and for a reference to the EST section, see www.ncbi.nlm.nih.gov/dbEST/; a general reference to dbEST, the EST database in GenBank, may be found in Boguski et al, Nat Genet. 1993 August; 4(4):332-3; all of which are hereby incorporated by reference as if fully set forth herein).

Novel splice variants were predicted using the LEADS clustering and assembly system as described in Sorek, R., Ast, G. & Graur, D. Alu-containing exons are alternatively spliced. Genome Res 12, 1060-7 (2002); U.S. Pat. No. 6,625,545; and U.S. patent application Ser. No. 10/426,002, published as US20040101876 on May 27, 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

A brief explanation is provided with regard to the method of selecting the candidates. However, it should be noted that this explanation is provided for descriptive purposes only, and is not intended to be limiting in any way. The potential markers were identified by a computational process that was designed to find genes and/or their splice variants that are specifically expressed in cardiac tissue, as opposed to other types of tissues and also particularly as opposed to muscle tissue, by using databases of expressed sequences. Various parameters related to the information in the EST libraries, determined according to classification by library annotation, were used to assist in locating genes and/or splice variants thereof that are specifically and/or differentially expressed in heart tissues. The detailed description of the selection method and of these parameters is presented in Example 1 below.

### Part I—Cardiac Disease Markers

### Example 1

### Identification of Differentially Expressed Gene Products

### Algorithm

In order to distinguish between differentially expressed gene products and constitutively expressed genes (i.e., house keeping genes), an algorithm based on an analysis of frequencies was configured. A specific algorithm for identification of transcripts specifically expressed in heart tissue is described hereinbelow.

EST Analysis

ESTs were taken from the following main sources: libraries contained in Genbank version 136 (Jun. 15, 2003 ftp.ncbi.nih.gov/genbank/release.notes/gb136.release.notes) and Genbank version 139 (December 2003); and from the LifeSeq library of Incyte Corporation (ESTs only; Wilmington, Del., USA). With regard to GenBank sequences, the human EST sequences from the EST (GBEST) section were used.

Library annotation—EST libraries were manually classified according to:

1. Tissue origin
2. Biological source—Examples of frequently used biological sources for construction of EST libraries include cancer cell-lines; normal tissues; cancer tissues; foetal tissues; and others such as normal cell lines and pools of normal cell-lines, cancer cell-lines and combinations thereof. A specific description of abbreviations used below with regard to these tissues/cell lines etc is given above.
3. Protocol of library construction—various methods are known in the art for library construction including normalized library construction; non-normalized library construction; subtracted libraries; ORESTES and others (described in the annotation available in Genbank). It will be appreciated that at times the protocol of library construction is not indicated in the information available about that library.

The following rules were followed:

EST libraries originating from identical biological samples were considered as a single library.

EST libraries which included above-average levels of contamination, such as DNA contamination for example, were eliminated. The presence of such contamination was determined as follows. For each library, the number of unspliced ESTs that are not fully contained within other spliced sequences was counted. If the percentage of such sequences (as compared to all other sequences) was at least 4 standard deviations above the average for all libraries being analyzed, this library was tagged as being contaminated and was eliminated from further consideration in the below analysis (see also Sorek, R. & Safer, H. M. A novel algorithm for computational identification of contaminated EST libraries. Nucleic Acids Res 31, 1067-74 (2003) for further details).

Clusters (genes) having at least five sequences including at least two sequences from the tissue of interest were analyzed. Splice variants were identified by using the LEADS software package as described above.

### Example 2

### Identification of Heart Tissue Specific Genes

For detection of heart tissue specific clusters, heart tissue libraries/sequences were compared to the total number of libraries/sequences in the cluster and in Genebank, and to the relevant numbers for muscle tissue libraries/sequences. Statistical tools were employed to identify clusters that were heart tissue specific, both as compared to all other tissues and also in comparison to muscle tissue.

The algorithm—for each tested tissue T and for each tested cluster the following were examined:

1. Each cluster includes at least 2 libraries from the tissue T. At least 3 clones (weighed—as described above) from tissue T in the cluster;

2. The following equation was then used to determine heart tissue-specific expression as compared to expression in all tissue types for a particular cluster:

$$\frac{t}{T} / \frac{n - t - m}{N - T - M}$$

in which n is the total number of ESTs available for a cluster, while N is the total number of ESTs available in all of the libraries considered in the analysis (effectively all ESTs in Genbank, except for those that were rejected as belonging to contaminated libraries). This ratio was preferably set to be at least about 8, although optionally the ratio could be set to be at least about 5.

3. The following equation was then used to determine heart tissue-specific expression vs. expression in skeletal muscle tissue for a particular cluster:

$$\frac{\frac{t}{T}}{\frac{m}{M}}$$

in which t represents the number of heart tissue-specific ESTs for the cluster, while T is the number of all heart tissue-specific ESTs in the analysis; m is the number of skeletal muscle tissue-specific ESTs for the cluster, while M is the number of all skeletal muscle tissue-specific ESTs in the analysis. This ratio was preferably set to be at least about 4, although optionally the ratio could be set to be at least about 2.
4. Fisher exact test P-values were computed for weighted clone counts to check that the counts are statistically significant according to the following function: F(t, T, n, N) which is the probability of a cluster actually being overexpressed in heart tissue, as compared to its overall level of expression. The P-value was preferably set to be less than about 1e-5, although optionally it could be set to be less than about 1e-3.
Selecting Candidates with Regard to Cancer

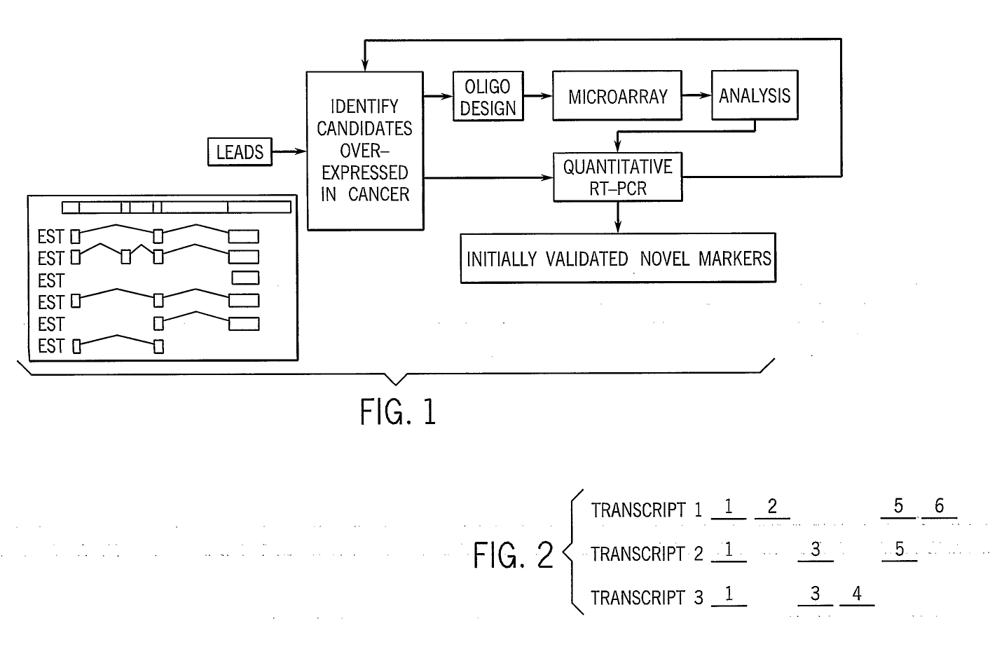

A brief explanation is provided with regard to a non-limiting method of selecting the candidates for cancer diagnostics. However, it should noted that this explanation is provided for descriptive purposes only, and is not intended to be limiting in any way. The potential markers were identified by a computational process that was designed to find genes and/or their splice variants that are over-expressed in tumor tissues, by using databases of expressed sequences. Various parameters related to the information in the EST libraries, determined according to a manual classification process, were used to assist in locating genes and/or splice variants thereof that are over-expressed in cancerous tissues. The detailed description of the selection method is presented in Example 1 below. The cancer biomarkers selection engine and the following wet validation stages are schematically summarized in FIG. 1.

### Part II—Cancer Markers

### Example 1

### Identification of Differentially Expressed Gene Products

### Algorithm

In order to distinguish between differentially expressed gene products and constitutively expressed genes (i.e., house keeping genes) an algorithm based on an analysis of frequencies was configured. A specific algorithm for identification of transcripts over expressed in cancer is described hereinbelow.

Dry Analysis

Library annotation—EST libraries are manually classified according to:
(i) Tissue origin
(ii) Biological source—Examples of frequently used biological sources for construction of EST libraries include cancer cell-lines; normal tissues; cancer tissues; fetal tissues; and others such as normal cell lines and pools of normal cell-lines, cancer cell-lines and combinations thereof. A specific description of abbreviations used below with regard to these tissues/cell lines etc is given above.
(iii) Protocol of library construction—various methods are known in the art for library construction including normalized library construction; non-normalized library construction; subtracted libraries; ORESTES and others. It will be appreciated that at times the protocol of library construction is not indicated.

The following rules are followed:

EST libraries originating from identical biological samples are considered as a single library.

EST libraries which include above-average levels of DNA contamination are eliminated.

Dry computation—development of engines which are capable of identifying genes and splice variants that are temporally and spacially expressed.

Clusters (genes) having at least five sequences including at least two sequences from the tissue of interest are analyzed.

### Example 2

### Identification of Genes Over Expressed in Cancer

Two different scoring algorithms were developed.

Libraries score—candidate sequences which are supported by a number of cancer libraries, are more likely to serve as specific and effective diagnostic markers.

The basic algorithm—for each cluster the number of cancer and normal libraries contributing sequences to the cluster was counted. Fisher exact test was used to check if cancer libraries are significantly over-represented in the cluster as compared to the total number of cancer and normal libraries.

Library counting: Small libraries (e.g., less than 1000 sequences) were excluded from consideration unless they participate in the cluster. For this reason, the total number of libraries is actually adjusted for each cluster.

Clones no. score—Generally, when the number of ESTs is much higher in the cancer libraries relative to the normal libraries it might indicate actual over-expression.

The algorithm—

Clone counting: For counting EST clones each library protocol class was given a weight based on our belief of how much the protocol reflects actual expression levels:

(i) non-normalized: 1

(ii) normalized: 0.2

(iii) all other classes: 0.1

Clones number score—The total weighted number of EST clones from cancer libraries was compared to the EST clones from normal libraries. To avoid cases where one library contributes to the majority of the score, the contribution of the library that gives most clones for a given cluster was limited to 2 clones.

The score was computed as

$$\frac{\frac{c + 1}{C}}{\frac{n + 1}{N}}$$

where:

c—weighted number of “cancer” clones in the cluster.

C—weighted number of clones in all “cancer” libraries.

n—weighted number of “normal” clones in the cluster.

N—weighted number of clones in all “normal” libraries.

Clones number score significance—Fisher exact test was used to check if EST clones from cancer libraries are significantly over-represented in the cluster as compared to the total number of EST clones from cancer and normal libraries.

Two search approaches were used to find either general cancer-specific candidates or tumor specific candidates.
Libraries/sequences originating from tumor tissues are counted as well as libraries originating from cancer cell-lines (“normal” cell-lines were ignored).
Only libraries/sequences originating from tumor tissues are counted

### Example 3

### Identification of Tissue Specific Genes

For detection of tissue specific clusters, tissue libraries/sequences were compared to the total number of libraries/sequences in cluster. Similar statistical tools to those described in above were employed to identify tissue specific genes. Tissue abbreviations are the same as for cancerous tissues, but are indicated with the header “normal tissue”.

The algorithm—for each tested tissue T and for each tested cluster the following were examined:

1. Each cluster includes at least 2 libraries from the tissue T. At least 3 clones (weighed—as described above) from tissue T in the cluster; and

2. Clones from the tissue T are at least 40% from all the clones participating in the tested cluster

Fisher exact test P-values were computed both for library and weighted clone counts to check that the counts are statistically significant.

### Example 4

### Identification of Splice Variants Over Expressed in Cancer of Clusters which are not Over Expressed in Cancer

Cancer-Specific Splice Variants Containing a Unique Region were Identified.

Identification of Unique Sequence Regions in Splice Variants

A Region is defined as a group of adjacent exons that always appear or do not appear together in each splice variant.

A “segment” (sometimes referred also as “seg” or “node”) is defined as the shortest contiguous transcribed region without known splicing inside.

Only reliable ESTs were considered for region and segment analysis. An EST was defined as unreliable if:

(i) Unspliced;

(ii) Not covered by RNA;

(iii) Not covered by spliced ESTs; and

(iv) Alignment to the genome ends in proximity of long poly-A stretch or starts in proximity of long poly-T stretch.

Only reliable regions were selected for further scoring. Unique sequence regions were considered reliable if:

(i) Aligned to the genome; and

(ii) Regions supported by more than 2 ESTs.

The algorithm

Each unique sequence region divides the set of transcripts into 2 groups:

(i) Transcripts containing this region (group TA).

(ii) Transcripts not containing this region (group TB).

The set of EST clones of every cluster is divided into 3 groups:

(i) Supporting (originating from) transcripts of group TA (S1).

(ii) Supporting transcripts of group TB (S2).

(iii) Supporting transcripts from both groups (S3).

Library and clones number scores described above were given to S1 group.

Fisher Exact Test P-values were used to check if:

S1 is significantly enriched by cancer EST clones compared to S2; and

S1 is significantly enriched by cancer EST clones compared to cluster background (S1+S2+S3).

Identification of unique sequence regions and division of the group of transcripts accordingly is illustrated in FIG. 2. Each of these unique sequence regions corresponds to a segment, also termed herein a “node”.

Region 1: common to all transcripts, thus it is not considered; Region 2: specific to Transcript 1: T_{—}1 unique regions (2+6) against T_{—}2+3 unique regions (3+4); Region 3: specific to Transcripts 2+3: T_{—}2+3 unique regions (3+4) against TI unique regions (2+6); Region 4: specific to Transcript 3: T_{—}3 unique regions (4) against T1+2 unique regions (2+5+6); Region 5: specific to Transcript 1+2: T_{—}1+2 unique regions (2+5+6) against T3 unique regions (4); Region 6: specific to Transcript 1: same as region 2.

### Example 5

### Identification of Cancer Specific Splice Variants of Genes Over Expressed in Cancer

A search for EST supported (no mRNA) regions for genes of:

(i) known cancer markers

(ii) Genes shown to be over-expressed in cancer in published micro-array experiments.

Reliable EST supported-regions were defined as supported by minimum of one of the following:

(i) 3 spliced ESTs; or

(ii) 2 spliced ESTs from 2 libraries;

(iii) 10 unspliced ESTs from 2 libraries, or

(iv) 3 libraries.

Oligonucleotide-Based Micro-Array Experiment Protocol—

### Microarray Fabrication

Microarrays (chips) were printed by pin deposition using the MicroGrid II MGII 600 robot from BioRobotics Limited (Cambridge, UK). 50-mer oligonucleotides target sequences were designed by Compugen Ltd (Tel-Aviv, Ill.) as described by A. Shoshan et al, “Optical technologies and informatics”, Proceedings of SPIE. Vol 4266, pp. 86-95 (2001). The designed oligonucleotides were synthesized and purified by desalting with the Sigma-Genosys system (The Woodlands, Tex., US) and all of the oligonucleotides were joined to a C6 amino-modified linker at the 5′ end, or being attached directly to CodeLink slides (Cat #25-6700-01. Amersham Bioscience, Piscataway, N.J., US). The 50-mer oligonucleotides, forming the target sequences, were first suspended in Ultra-pure DDW (Cat #01-866-1A Kibbutz Beit-Haemek, Israel) to a concentration of 50 μM. Before printing the slides, the oligonucleotides were resuspended in 300 mM sodium phosphate (pH 8.5) to final concentration of 150 mM and printed at 35-40% relative humidity at 21° C.

Each slide contained a total of 9792 features in 32 subarrays. Of these features, 4224 features were sequences of interest according to the present invention and negative controls that were printed in duplicate. An additional 288 features (96 target sequences printed in triplicate) contained housekeeping genes from Human Evaluation Library2, Compugen Ltd, Israel. Another 384 features are E. coli spikes 1-6, which are oligos to E-Coli genes which are commercially available in the Array Control product (Array control—sense oligo spots, Ambion Inc. Austin, Tex. Cat #1781, Lot #112K06).

### Post-Coupling Processing of Printed Slides

After the spotting of the oligonucleotides to the glass (CodeLink) slides, the slides were incubated for 24 hours in a sealed saturated NaCl humidification chamber (relative humidity 70-75%).

Slides were treated for blocking of the residual reactive groups by incubating them in blocking solution at 50° C. for 15 minutes (10 ml/slide of buffer containing 0.1M Tris, 50 mM ethanolamine, 0.1% SDS). The slides were then rinsed twice with Ultra-pure DDW (double distilled water). The slides were then washed with wash solution (10 ml/slide. 4×SSC, 0.1% SDS)) at 50° C. for 30 minutes on the shaker. The slides were then rinsed twice with Ultra-pure DDW, followed by drying by centrifugation for 3 minutes at 800 rpm.

Next, in order to assist in automatic operation of the hybridization protocol, the slides were treated with Ventana Discovery hybridization station barcode adhesives. The printed slides were loaded on a Bio-Optica (Milan, Italy) hematology staining device and were incubated for 10 minutes in 50 ml of 3-Aminopropyl Triethoxysilane (Sigma A3648 lot #122K589). Excess fluid was dried and slides were then incubated for three hours in 20 mm/Hg in a dark vacuum desiccator (Pelco 2251, Ted Pella, Inc. Redding Calif.).

The following protocol was then followed with the Genisphere 900-RP (random primer), with mini elute columns on the Ventana Discovery HybStation™, to perform the microarray experiments. Briefly, the protocol was performed as described with regard to the instructions and information provided with the device itself. The protocol included cDNA synthesis and labeling. cDNA concentration was measured with the TBS-380 (Turner Biosystems. Sunnyvale, Calif.) PicoFlour, which is used with the OliGreen ssDNA Quantitation reagent and kit.

Hybridization was performed with the Ventana Hybridization device, according to the provided protocols (Discovery Hybridization Station Tuscon Ariz.).

The slides were then scanned with GenePix 4000B dual laser scanner from Axon Instruments Inc, and analyzed by GenePix Pro 5.0 software.

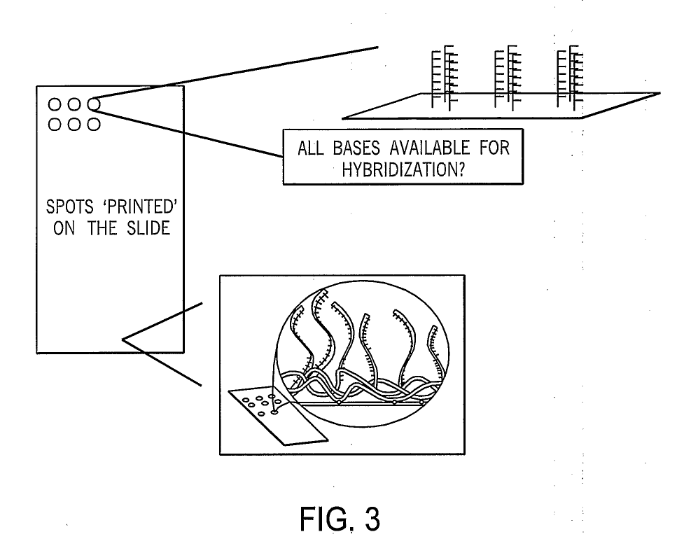

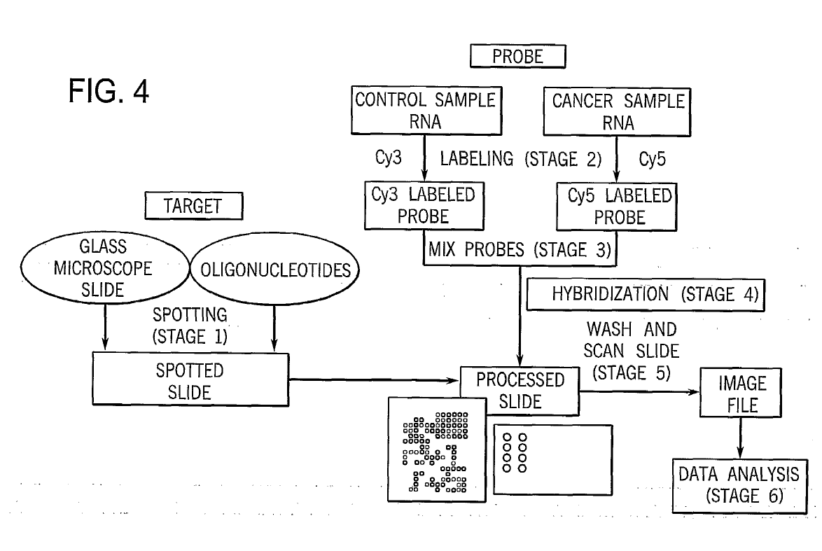

Schematic summary of the oligonucleotide based microarray fabrication and the experimental flow is presented in FIGS. 3 and 4.

Briefly, as shown in FIG. 3, DNA oligonucleotides at 25 uM were deposited (printed) onto Amersham ‘CodeLink’ glass slides generating a well defined ‘spot’. These slides are covered with a long-chain, hydrophilic polymer chemistry that creates an active 3-D surface that covalently binds the DNA oligonucleotides 5′-end via the

C6-amine modification. This binding ensures that the full length of the DNA oligonucleotides is available for hybridization to the cDNA and also allows lower background, high sensitivity and reproducibility.

FIG. 4 shows a schematic method for performing the microarray experiments. It should be noted that stages on the left-hand or right-hand side may optionally be performed in any order, including in parallel, until stage 4 (hybridization). Briefly, on the left-hand side, the target oligonucleotides are being spotted on a glass microscope slide (although optionally other materials could be used) to form a spotted slide (stage 1). On the right hand side, control sample RNA and cancer sample RNA are Cy3 and Cy5 labeled, respectively (stage 2), to form labeled probes. It should be noted that the control and cancer samples come from corresponding tissues (for example, normal prostate tissue and cancerous prostate tissue). Furthermore, the tissue from which the RNA was taken is indicated below in the specific examples of data for particular clusters, with regard to overexpression of an oligonucleotide from a “chip” (microarray), as for example “prostate” for chips in which prostate cancerous tissue and normal tissue were tested as described above. In stage 3, the probes are mixed. In stage 4, hybridization is performed to form a processed slide. In stage 5, the slide is washed and scanned to form an image file, followed by data analysis in stage 6.

Diseases and Conditions that May be Diagnosed with One or More Variant(s) According to the Present Invention

### Cardiovascular and Cerebrovascular Conditions

Various examples are listed below for conditions that affect the vascular system, including various cardiovascular and cerebrovascular conditions, for which one or more variants according to the present invention may have a diagnostic utility.

### Myocardial Infarction

N56180 variants, S67314 variants, HUMNATPEP variants, HUMCDDANF variants, HSACMHCP variants, HSCREACT variants and/or Z3624 variants are potential markers for myocardial infarction. Other conditions that may be diagnosed by these markers or variants of them include but are not limited to the presence, risk and/or extent of the following:
1. Myocarditis—in myocarditis cardiac muscle cells can go through cell lysis and leakage with the release of intracellular content to the extracellular space and blood, a similar process as happens in myocardial infarction (see also extended description below).
2. Angina—stable or unstable, as the reduction of oxygen delivery to part of the heart often leads to local ischemic conditions that facilitate leakage of intracellular content.
3. Traumatic injury to myocardial tissue—blunt or penetrating, may also result in myocardial cell leakage.
4. Opening an occluded coronary artery following thrombolytic therapy—If such treatment is successful, proteins and other products of the local tissue are washed into the blood and can be detected there.
5. Cardiomyopathy—which is characterized by slow degeneration of the heart muscle (see also extended description below).
6. Myocardial injury after rejection of heart transplant.
7. Congestive heart failure where heart myocytes slowly degenerate (as had been shown for Troponin-I; see also extended description below).
8. Future cardiovascular disease (as a risk factor).
9. Conditions which have similar clinical symptoms as myocardial infarction and where the differential diagnosis between them and myocardial infarction is of clinical importance including but not limited to:
   a. Clinical symptoms resulting from lung related tissue (e.g. Pleuritis, pulmonary embolism)
   b. Musculoskeletal origin of pain
   c. Clinical symptoms resulting from heart related tissue which are not due to myocardial infarction, e.g. acute pericarditis
   d. Upper abdominal pain from abdominal organs including but nor limited to esophagitis, gastro-esophageal reflux, gastritis, gastric ulcer, duodenitis, duodenal ulcer, enteritis, gastroenteritis, cholecystitis, cholelithiasis, cholangiolithiasis, pancreatitis, splenic infarction, splenic trauma, Aortic dissection.

One or more of these markers (variants according to the present invention) may optionally be used a tool to decide on treatment options e.g. anti platelet inhibitors (as has been shown for Troponin-I); as a tool in the assessment of pericardial effusion; and/or as a tool in the assessment of endocarditis and/or rheumatic fever, where progressive damage to the heart muscle may occur.

### Acute and Chronic Inflammation and Risk Factors for CVS Diseases

N56180 variants, S67314 variants, HUMNATPEP variants, HUMCDDANF variants, HSACMHCP variants, HSCREACT variants and/or Z3624 variants are potential markers for inflammation, including a spectrum of diseases where an inflammatory process plays a substantial role. In addition CRP levels and in particular baseline levels serve as a risk factor for various diseases, particularly cardiovascular diseases where inflammation is thought to participate in the pathogenesis. Conditions that may be diagnosed by these markers or variants of them include but are not limited to the presence, risk and/or extent of the following:
1. Conditions that entail an inflammatory process that involves blood vessels including but not limited to hypercholesterolemia, diabetes, atherosclerosis, inflammation that involves blood vessels—whether acute or chronic including but not limited to the coronary arteries and blood vessels of the brain, myocardial infarction, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behçet syndrome, and their complications including but not limited to coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina.
2. Rheumatic/autoimmune diseases that involve systemic immune reaction including but not limited to rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus.
3. Acute and/or chronic infective processes that involve systemic immune reaction including but not limited to pneumonia, bacteremia, sepsis, pyelonephritis, cellulitis, osteomyelitis, meningitis and viral hepatitis.
4. Malignant and idiopathic processes that involve systemic immune reaction and/or proliferation of immune cells including but not limited to granulomatous disorders, Wegener's granulomatosis, lymphomatoid granulomatosis/polymorphic reticulosis, idiopathic midline granuloma, multiple myeloma, Waldenstrom's macroglobulinemia, Castleman's disease, amyloidosis, lymphoma, histiocytosis, renal cell carcinoma and paraneoplastic syndromes.
5. Conditions where CRP was shown to have a positive correlation with the presence of the condition including but not limited to weight loss, anorexia-cachexia syndrome, extent of disease, recurrence in advanced cancer, diabetes (types 1 & 2), obesity, hypertension, preterm delivery.
6. Conditions which have similar symptoms, signs and complications as the conditions above and where the differential diagnosis between them and the conditions above is of clinical importance including but not limited to:
   a. Other (non vascular) causes of heart disease, renal disease and cerebral disease.
   b. Other (non rheumatic) causes of arthropathy and musculoskeletal pain.
   c. Other causes of non-specific symptoms and signs such as fever of unknown origin, loss of appetite, weight loss, nonspecific pains, breathing difficulties and anxiety.

### Stroke

Stroke is a manifestation of vascular injury to the brain which is commonly secondary to atherosclerosis or hypertension, and is the third leading cause of death (and the second most common cause of neurologic disability) in the United States. Preferred marker(s) for diagnosis of stroke and related conditions as described herein may optionally be selected from the group consisting of IL-1ra, C-reactive protein (CRP) or variants thereof as described herein with regard to cluster HSCREACT, von Willebrand factor (vWF), vascular endothelial growth factor (VEGF) or variants thereof as described with regard to U.S. Pat. No. 6,783,954 (previously incorporated by reference), matrix metalloprotease-9 (MMP-9), neural cell adhesion molecule (NCAM) or variants thereof as described with regard to PCT Application No. WO 01/29215 (incorporated by reference as if fully set forth herein), BNP or variants thereof as described herein with regard to cluster HUMNATPEP, markers from cluster N56180, S67314, HUMCDDANF and/or HSACMHCP, and caspase-3, or markers related thereto.

Stroke is a pathological condition with acute onset that is caused by the occlusion or rupture of a vessel supplying blood, and thus oxygen and nutrients, to the brain. The immediate area of injury is referred to as the “core,” which contains brain cells that have died as a result of ischemia or physical damage. The “penumbra” is composed of brain cells that are neurologically or chemically connected to cells in the core. Cells within the penumbra are injured, but still have the ability to completely recover following removal of the insult caused during stroke. However, as ischemia or bleeding from hemorrhage continues, the core of dead cells can expand from the site of insult, resulting in a concurrent expansion of cells in the penumbra. The initial volume and rate of core expansion is related to the severity of the stroke and, in most cases, neurological outcome.

The brain contains two major types of cells, neurons and glial cells. Neurons are the most important cells in the brain, and are responsible for maintaining communication within the brain via electrical and chemical signaling. Glial cells function mainly as structural components of the brain, and they are approximately 10 times more abundant than neurons. Glial cells of the central nervous system (CNS) are astrocytes and oligodendrocytes. Astrocytes are the major interstitial cells of the brain, and they extend cellular processes that are intertwined with and surround neurons, isolating them from other neurons. Astrocytes can also form “end feet” at the end of their processes that surround capillaries. Oligodendrocytes are cells that form myelin sheathes around axons in the CNS. Each oligodendrocyte has the ability to ensheathe up to 50 axons. Schwann cells are glial cells of the peripheral nervous system (PNS). Schwann cells form myelin sheathes around axons in the periphery, and each Schwann cell ensheathes a single axon.

Cell death during stroke occurs as a result of ischemia or physical damage to the cells of the CNS. During ischemic stroke, an infarct occurs, greatly reducing or stopping blood flow beyond the site of infarction. The zone immediately beyond the infarct soon lacks suitable blood concentrations of the nutrients essential for cell survival. Cells that lack nutrients essential for the maintenance of important functions like metabolism soon perish. Hemorrhagic stroke can induce cell death by direct trauma, elevation in intracranial pressure, and the release of damaging biochemical substances in blood. When cells die, they release their cytosolic contents into the extracellular milieu.

The barrier action of tight junctions between the capillary endothelial cells of the central nervous system is referred to as the “blood-brain barrier”. This barrier is normally impermeable to proteins and other molecules, both large and small. In other tissues such as skeletal, cardiac, and smooth muscle, the junctions between endothelial cells are loose enough to allow passage of most molecules, but not proteins.

Substances that are secreted by the neurons and glial cells (intracellular brain compartment) of the central nervous system (CNS) can freely pass into the extracellular milieu (extracellular brain compartment). Likewise, substances from the extracellular brain compartment can pass into the intracellular brain compartment. The passage of substances between the intracellular and extracellular brain compartments are restricted by the normal cellular mechanisms that regulate substance entry and exit. Substances that are found in the extracellular brain compartment also are able to pass freely into the cerebrospinal fluid, and vice versa. This movement is controlled by diffusion.

The movement of substances between the vasculature and the CNS is restricted by the blood-brain barrier. This restriction can be circumvented by facilitated transport mechanisms in the endothelial cells that transport, among other substances, nutrients like glucose and amino acids across the barrier for consumption by the cells of the CNS. Furthermore, lipid-soluble substances such as molecular oxygen and carbon dioxide, as well as any lipid-soluble drugs or narcotics can freely diffuse across the blood-brain barrier.

Depending upon their size, specific markers of neural tissue injury that are released from injured brain cells during stroke or other neuropathies will only be found in peripheral blood when CNS injury is coupled with or followed by an increase in the permeability of the blood-brain barrier. This is particularly true of larger molecules. Smaller molecules may appear in the peripheral blood as a result of passive diffusion, active transport, or an increase in the permeability of the blood-brain barrier. Increases in blood-brain barrier permeability can arise as a result of physical disruption in cases such as tumor invasion and extravasation or vascular rupture, or as a result of endothelial cell death due to ischemia. During stroke, the blood-brain barrier is compromised by endothelial cell death, and any cytosolic components of dead cells that are present within the local extracellular milieu can enter the bloodstream.

Therefore, specific markers of neural tissue injury may also be found in the blood or in blood components such as serum and plasma, as well as the CSF of a patient experiencing stroke or TIAs. Furthermore, clearance of the obstructing object in ischemic stroke can cause injury from oxidative insult during reperfusion, and patients with ischemic stroke can sometimes experience hemorrhagic transformation as a result of reperfusion or thrombolytic therapy. Additionally, injury can be caused by vasospasm, which is a focal or diffuse narrowing of the large capacity arteries at the base of the brain following hemorrhage. The increase in blood-brain barrier permeability is related to the insult severity, and its integrity is reestablished following the resolution of insult. Specific markers of neural tissue injury will only be present in peripheral blood if there has been a sufficient increase in the permeability of the blood-brain barrier that allows these large molecules to diffuse across. In this regard, most specific markers of neural tissue injury can be found in cerebrospinal fluid after stroke or any other neuropathy that affects the CNS. Furthermore, many investigations of coagulation or fibrinolysis markers in stroke are performed using cerebrospinal fluid.

The coagulation cascade in stroke is now described. There are essentially two mechanisms that are used to halt or prevent blood loss following vessel injury. The first mechanism involves the activation of platelets to facilitate adherence to the site of vessel injury. The activated platelets then aggregate to form a platelet plug that reduces or temporarily stops blood loss. The processes of platelet aggregation, plug formation and tissue repair are all accelerated and enhanced by numerous factors secreted by activated platelets. Platelet aggregation and plug formation is mediated by the formation of a fibrinogen bridge between activated platelets. Concurrent activation of the second mechanism, the coagulation cascade, results in the generation of fibrin from fibrinogen and the formation of an insoluble fibrin clot that strengthens the platelet plug.

The coagulation cascade is an enzymatic pathway that involves numerous serine proteinases normally present in an inactive, or zymogen, form. The presence of a foreign surface in the vasculature or vascular injury results in the activation of the intrinsic and extrinsic coagulation pathways, respectively. A final common pathway is then followed, which results in the generation of fibrin by the serine proteinase thrombin and, ultimately, a crosslinked fibrin clot. In the coagulation cascade, one active enzyme is formed initially, which can activate other enzymes that active others, and this process, if left unregulated, can continue until all coagulation enzymes are activated. Fortunately, there are mechanisms in place, including fibrinolysis and the action of endogenous proteinase inhibitors that can regulate the activity of the coagulation pathway and clot formation.

Fibrinolysis is the process of proteolytic clot dissolution. In a manner analogous to coagulation, fibrinolysis is mediated by serine proteinases that are activated from their zymogen form. The serine proteinase plasmin is responsible for the degradation of fibrin into smaller degradation products that are liberated from the clot, resulting in clot dissolution. Fibrinolysis is activated soon after coagulation in order to regulate clot formation. Endogenous serine proteinase inhibitors also function as regulators of fibrinolysis.

The presence of a coagulation or fibrinolysis marker in cerebrospinal fluid would indicate that activation of coagulation or fibrinolysis, depending upon the marker used, coupled with increased permeability of the blood-brain barrier has occurred. In this regard, more definitive conclusions regarding the presence of coagulation or fibrinolysis markers associated with acute stroke may be obtained using cerebrospinal fluid.

Platelets are round or oval disks with an average diameter of 2-4 microns that are normally found in blood at a concentration of 200,000-300,000/microliter. They play an essential role in maintaining hemostasis by maintaining vascular integrity, initially stopping bleeding by forming a platelet plug at the site of vascular injury, and by contributing to the process of fibrin formation to stabilize the platelet plug. When vascular injury occurs, platelets adhere to the site of injury and each other and are stimulated to aggregate by various agents released from adherent platelets and injured endothelial cells. This is followed by the release reaction, in which platelets secrete the contents of their intracellular granules, and formation of the platelet plug. The formation of fibrin by thrombin in the coagulation cascade allows for consolidation of the plug, followed by clot retraction and stabilization of the plug by crosslinked fibrin. Active thrombin, generated in the concurrent coagulation cascade, also has the ability to induce platelet activation and aggregation.

The coagulation cascade can be activated through either the extrinsic or intrinsic pathways. These enzymatic pathways share one final common pathway. The result of coagulation activation is the formation of a crosslinked fibrin clot. Fibrinolysis is the process of proteolytic clot dissolution that is activated soon after coagulation activation, perhaps in an effort to control the rate and amount of clot formation. Urokinase-type plasminogen activator (uPA) and tissue-type plasminogen activator (tPA) proteolytically cleave plasminogen, generating the active serine proteinase plasmin. Plasmin proteolytically digests crosslinked fibrin, resulting in clot dissolution and the production and release of fibrin degradation products.

The first step of the common pathway of the coagulation cascade involves the proteolytic cleavage of prothrombin by the factor Xa/factor Va prothrombinase complex to yield active thrombin. Thrombin is a serine proteinase that proteolytically cleaves fibrinogen to form fibrin, which is ultimately integrated into a crosslinked network during clot formation.

Stroke can be categorized into two broad types, “ischemic stroke” and “hemorrhagic stroke.” Additionally, a patient may experience transient ischemic attacks, which are in turn a high risk factor for the future development of a more severe episode.

Ischemic stroke encompasses thrombotic, embolic, lacunar and hypoperfusion types of strokes. Thrombi are occlusions of arteries created in situ within the brain, while emboli are occlusions caused by material from a distant source, such as the heart and major vessels, often dislodged due to myocardial infarct or atrial fibrillation. Less frequently, thrombi may also result from vascular inflammation due to disorders such as meningitis. Thrombi or emboli can result from atherosclerosis or other disorders, for example, arteritis, and lead to physical obstruction of arterial blood supply to the brain. Lacunar stroke refers to an infarct within non-cortical regions of the brain. Hypoperfusion embodies diffuse injury caused by non-localized cerebral ischemia, typically caused by myocardial infarction and arrhythmia.

The onset of ischemic stroke is often abrupt, and can become an “evolving stroke” manifested by neurologic deficits that worsen over a 24-48 hour period. In evolving stroke, “stroke-associated symptom(s)” commonly include unilateral neurologic dysfunction which extends progressively, without producing headache or fever. Evolving stroke may also become a “completed stroke,” in which symptoms develop rapidly and are maximal within a few minutes.

Hemorrhagic stroke is caused by intracerebral or subarachnoid hemorrhage, i.e., bleeding into brain tissue, following blood vessel rupture within the brain. Intracerebral and subarachnoid hemorrhage are subsets of a broader category of hemorrhage referred to as intracranial hemorrhage. Intracerebral hemorrhage is typically due to chronic hypertension, and a resulting rupture of an arteriosclerotic vessel. Stroke-associated symptom(s) of intracerebral hemorrhage are abrupt, with the onset of headache and steadily increasing neurological deficits. Nausea, vomiting, delirium, seizures and loss of consciousness are additional common stroke-associated symptoms.

In contrast, most subarachnoid hemorrhage is caused by head trauma or aneurysm rupture which is accompanied by high pressure blood release which also causes direct cellular trauma. Prior to rupture, aneurysms may be asymptomatic, or occasionally associated with tension or migraine headaches. However, headache typically becomes acute and severe upon rupture, and may be accompanied by varying degrees of neurological deficit, vomiting, dizziness, and altered pulse and respiratory rates.

Transient ischemic attacks (TIAs) have a sudden onset and brief duration, typically 2-30 minutes. Most TIAs are due to emboli from atherosclerotic plaques, often originating in the arteries of the neck, and can result from brief interruptions of blood flow. The symptoms of TIAs are identical to those of stroke, but are only transient. Concomitant with underlying risk factors, patients experiencing TIAs are at a markedly increased risk for stroke.

Current diagnostic methods for stroke include costly and time-consuming procedures such as noncontrast computed tomography (CT) scan, electrocardiogram, magnetic resonance imaging (MRI), and angiography. Determining the immediate cause of stroke and differentiating ischemic from hemorrhagic stroke is difficult. CT scans can detect parenchymal bleeding greater than 1 cm and 95% of all subarachnoid hemorrhages. CT scan often cannot detect ischemic strokes until 6 hours from onset, depending on the infarct size. MRI may be more effective than CT scan in early detection of ischemic stroke, but it is less accurate at differentiating ischemic from hemorrhagic stroke, and is not widely available. An electrocardiogram (ECG) can be used in certain circumstances to identify a cardiac cause of stroke. Angiography is a definitive test to identify stenosis or occlusion of large and small cranial blood vessels, and can locate the cause of subarachnoid hemorrhages, define aneurysms, and detect cerebral vasospasm. It is, however, an invasive procedure that is also limited by cost and availability. Coagulation studies can also be used to rule out a coagulation disorder (coagulopathy) as a cause of hemorrhagic stroke.

Immediate diagnosis and care of a patient experiencing stroke can be critical. For example, tissue plasminogen activator (TPA) given within three hours of symptom onset in ischemic stroke is beneficial for selected acute stroke patients. Alternatively, patients may benefit from anticoagulants (e.g., heparin) if they are not candidates for TPA therapy. In contrast, thrombolytics and anticoagulants are strongly contraindicated in hemorrhagic strokes. Thus, early differentiation of ischemic events from hemorrhagic events is imperative. Moreover, delays in the confirmation of stroke diagnosis and the identification of stroke type limit the number of patients that may benefit from early intervention therapy. Finally, there are currently no diagnostic methods that can identify a TIA, or predict delayed neurological deficits which are often detected at a time after onset concurrent with the presentation of symptoms.

Accordingly, there is a present need in the art for a rapid, sensitive and specific diagnostic assay for stroke and TIA that can also differentiate the stroke type and identify those individuals at risk for delayed neurological deficits. Such a diagnostic assay would greatly increase the number of patients that can receive beneficial stroke treatment and therapy, and reduce the costs associated with incorrect stroke diagnosis.

The present invention relates to the identification and use of diagnostic markers for stroke and neural tissue injury. The methods and compositions described herein can meet the need in the art for rapid, sensitive and specific diagnostic assay to be used in the diagnosis and differentiation of various forms of stroke and TIAs. Moreover, the methods and compositions of the present invention can also be used to facilitate the treatment of stroke patients and the development of additional diagnostic and/or prognostic indicators.

In various aspects, the invention relates to materials and procedures for identifying markers that are associated with the diagnosis, prognosis, or differentiation of stroke and/or TIA in a patient; to using such markers in diagnosing and treating a patient and/or to monitor the course of a treatment regimen; to using such markers to identify subjects at risk for one or more adverse outcomes related to stroke and/or TIA; and for screening compounds and pharmaceutical compositions that might provide a benefit in treating or preventing such conditions.

In a first aspect, the invention discloses methods for determining a diagnosis or prognosis related to stroke, or for differentiating between types of strokes and/or TIA. These methods comprise analyzing a test sample obtained from a subject for the presence or amount of one or more markers for neural tissue injury. These methods can comprise identifying one or more markers, the presence or amount of which is associated with the diagnosis, prognosis, or differentiation of stroke and/or TIA. Once such marker(s) are identified, the level of such marker(s) in a sample obtained from a subject of interest can be measured. In certain embodiments, these markers can be compared to a level that is associated with the diagnosis, prognosis, or differentiation of stroke and/or TIA. By correlating the subject's marker level(s) to the diagnostic marker level(s), the presence or absence of stroke, the probability of future adverse outcomes, etc., in a patient may be rapidly and accurately determined.

In a related aspect, the invention discloses methods for determining the presence or absence of a disease in a subject that is exhibiting a perceptible change in one or more physical characteristics (that is, one or more “symptoms”) that are indicative of a plurality of possible etiologies underlying the observed symptom(s), one of which is stroke. These methods comprise analyzing a test sample obtained from the subject for the presence or amount of one or more markers selected to rule in or out stroke, or one or more types of stroke, as a possible etiology of the observed symptom(s). Etiologies other than stroke that are within the differential diagnosis of the symptom(s) observed are referred to herein as “stroke mimics”, and marker(s) able to differentiate one or more types of stroke from stroke mimics are referred to herein as “stroke differential diagnostic markers”. The presence or amount of such marker(s) in a sample obtained from the subject can be used to rule in or rule out one or more of the following: stroke, thrombotic stroke, embolic stroke, lacunar stroke, hypoperfusion, intracerebral hemorrhage, and subarachnoid hemorrhage, thereby either providing a diagnosis (rule-in) and/or excluding a diagnosis (rule-out).

Obtaining information on the true time of onset can be critical, as early treatments have been reported to be critical for proper treatment. Obtaining this time-of-onset information may be difficult, and is often based upon interviews with companions of the stroke victim. Thus, in various embodiments, markers and marker panels are selected to distinguish the approximate time since stroke onset. For purposes of the present invention, the term “acute stroke” refers to a stroke that has occurred within the prior 12 hours, more preferably within the prior 6 hours, and most preferably within the prior 3 hours; while the term “non-acute stroke” refers to a stroke that has occurred more than 12 hours ago, preferably between 12 and 48 hours ago, and most preferably between 12 and 24 hours ago. Preferred markers for differentiating between acute and non-acute strokes, referred to herein as stroke “time of onset markers” are described hereinafter.

For markers appearing in the patent which are already linked to stroke, either ischemic or hemorrhagic, variants could also help to diagnose, directly or by elimination of other conditions including but not limited to:
1. Transient ischemic attack
2. Brain trauma, in case it is unclear whether accompanied by stroke or not
3. Migraine
4. Bleeding in any part of the brain or inside the skull that cause or didn't cause damage to brain tissue
5. Tumor
   In addition, such markers may help determine:
1. The time of stroke
2. The type of stroke
3. The extent of tissue damage as a result of the stroke
4. Response to immediate treatments that are meant to alleviate the extent of stroke and brain damage, when available.

With regard to stroke, according to preferred embodiments of the present invention, the panel may optionally and preferably provide diagnosis of stroke and indication if an ischemic stroke has occurred; diagnosis of stroke and indication if a hemorrhagic stroke has occurred; diagnosis of stroke, indication if an ischemic stroke has occurred, and indication if a hemorrhagic stroke has occurred; diagnosis of stroke and prognosis of a subsequent cerebral vasospasm; and diagnosis of stroke, indication if a hemorrhagic stroke has occurred, and prognosis of a subsequent cerebral vasospasm.

According to other optional embodiments of the present invention, there are provided methods of identifying a patient at risk for cerebral vasospasm. Such methods preferably comprise comparing an amount of one or more marker(s) predictive of a subsequent cerebral vasospasm in a test sample from a patient diagnosed with a subarachnoid hemorrhage. Such markers may be one or more markers related to blood pressure regulation, markers related to inflammation, markers related to apoptosis, and/or specific markers of neural tissue injury. As discussed herein, such marker may be used in panels comprising 2, 3, 4, 5, 6, 7, 8, 9, 10, 15, 20, or more or individual markers. Preferred marker(s) may be selected from the group consisting of IL-1ra, C-reactive protein (CRP) or variants thereof as described herein with regard to cluster HSCREACT, von Willebrand factor (vWF), vascular endothelial growth factor (VEGF) or variants thereof as described with regard to U.S. Pat. No. 6,783,954 (previously incorporated by reference), matrix metalloprotease-9 (MMP-9), neural cell adhesion molecule (NCAM) or variants thereof as described with regard to PCT Application No. WO 01/29215 (incorporated by reference as if fully set forth herein), BNP or variants thereof as described herein with regard to cluster HUMNATPEP, markers from cluster N56180, S67314, HUMCDDANF and/or HSACMHCP, and caspase-3, or markers related thereto. The levels of one or more markers may be compared to a predictive level of said marker(s), wherein said patient is identified as being at risk for cerebral vasospasm by a level of said marker(s) equal to or greater than said predictive level. In the alternative, a panel response value for a plurality of such markers may be determined, optionally considering a change in the level of one or more such markers as an additional independent marker.

According to yet other embodiments of the present invention, there are provided methods of differentiating ischemic stroke from hemorrhagic stroke using such marker panels.

### Cardiomyopathy and Myocarditis

Cardiomyopathy is a general diagnostic term designating primary myocardial disease which may progress to heart failure. Cardiomyopathies constitute a group of diseases in which the dominant feature is involvement of the heart muscle itself. In many cases, cardiomyopathies are of obscure or unknown aetiology, but in some cases the cause of the cardiomyopathy is known. For example, inflammatory cardiomyopathies may arise as a result of an infection by a viral, bacterial of parasitic organism. Cardiomyopathies may also result from a metabolic disorder such as a nutritional deficiency or by altered endocrine function. Other cardiomyopathies may be attributed to toxic substances, for example from alcohol or exposure to cobalt or lead. Still other types of cardiomyopathies may result from infiltration and deposition of abnormal cellular materials such as that known to occur during neoplastic infiltration or cardiac amyloidosis. Preferred marker(s) for diagnosis of cardiomyopathy and myocarditis, and related conditions as described herein, may optionally be selected from the group consisting of variants in N56180, S67314, HUMNATPEP, HUMCDDANF, HSACMHCP, HSCREACT or Z36249 clusters.

BNP levels have been shown to be elevated in specific cardiomyopathies. For example, BNP levels have been shown to be elevated in idiopathic dilated cardiomyopathy (Fruwald et al., 1999 Eur Heart J. 20: 1415-23), hypertrophic cardiomyopathy (Hamada et al., 1997 Clin Sci. (Colch) 94:21-8; Hasegawa et al., 1993 Circ. 88: 372-80), hypertrophic obstructive cardiomyopathy (Nishigaki et al., 1996 J. Am Coll Cardiol. 28:1234-42), dilated cardiomyopathy (Yasue et al., 1994 Circulation 90:195-203; Alterme et al., 1997 J. Heart Lung Transplant 16:765-73), genetic cardiomyopathy (Carnio et al., 1997 Regul Pept. 70:67-73) and in cardiac amyloidosis. However, none of these references disclose that BNP or ANF levels are elevated in other causes of cardiomyopathy including inflammatory cardiomyopathy that arise following or as a result of an infection.

Myocarditis is a condition relating to inflammation of the heart muscle. More specifically myocarditis is a disorder caused by inflammation of the myocytes, interstitium, vascular elements or the pericardium of the heart. Much like the cardiomyopathies, the causative agent of myocarditis may be known or unknown. However, it is known that myocarditis may arise as a complication during or after infection by various viral, bacterial or parasitic disease organisms. In North America, viruses (especially enteroviruses) are presumed to be the most common agents of myocarditis, whereas in South America, Chagas disease (American trypanosomia) produced by Trypanosoma cruzi is far more common. Patients with myocarditis may exhibit several symptoms including fever, and heart function problems, for example lower cardiac output. Myocarditis can also result from an inflammation without infection, due to an auto-immune process for example.

A number of infections and infectious agents are associated with cardiomyopathy and/or myocarditis. By the term infection it is meant any viral infection, rickettsial infection, bacterial infection, mycobacterial infection, spirochetal infection, fungal infection, parasitic infection or any other infection by any other infectious organism known in the art. The infection may directly cause cardiomyopathy or myocardits, or the infection may indirectly contribute to the development of cardiomyopathy or myocarditis.

N56180 variants, S67314 variants, HUMNATPEP variants, HUMCDDANF variants, HSACMHCP variants, HSCREACT variants and/or Z3624 variants are potential markers for cardiomyopathy and/or myocarditis.

### Congestive Heart Failure (CHF)

N56180 variants, S67314 variants, HUMNATPEP variants, HUMCDDANF variants, HSACMHCP variants, HSCREACT variants, HSTGFB1 variants and/or Z3624 variants are potential markers for CHF. Other conditions that may be diagnosed by these markers or variants of them include but are not limited to the presence, risk and/or extent of the following:
1. A risk factor for sudden cardiac death, from arrhythmia or any other heart related reason.
2. Rejection of a transplanted heart.
3. Conditions that lead to heart failure including but not limited to myocardial infarction, angina, arrhythmias, valvular diseases, atrial and/or ventricular septal defects.
4. Conditions that cause atrial and or ventricular wall volume overload. Wall stretch results in enhanced secretion of cardiac extracellular regulators. Such conditions include but are not limited to systemic arterial hypertension, pulmonary hypertension and pulmonary embolism.
5. Conditions which have similar clinical symptoms as heart failure and as states that cause atrial and or ventricular pressure-overload, where the differential diagnosis between these conditions to the latter is of clinical importance including but not limited to breathing difficulty and/or hypoxia due to pulmonary disease, anemia or anxiety.

### Cancerous Conditions

Various non-limiting examples are given below of cancerous conditions for which one or more variants according to the present invention may have a diagnostic utility.

### Breast Cancer

S57296, HUMGRP5E, T94936, and/or HSTGFB1 or variants as described herein or markers related thereto are potential markers for breast cancer. Other conditions that may be diagnosed by these markers or variants of them include but are not limited to the presence, risk and/or extent of the following:
1. The identification of a metastasis of unknown origin which originated from a primary breast cancer tumor.
2. In the assessment of lymphadenopathy, and in particular axillary lymphadenopathy.
3. Distinguishing between different types of breast cancer, therefore potentially affect treatment choice (e.g. as HER-2)
4. Differential diagnosis between a benign and malignant breast mass.
5. As a tool in the assessment of conditions affecting breast skin (e.g. Paget's disease) and their differentiation from breast cancer.
6. Differential diagnosis of breast pain or discomfort resulting from either breast cancer or other possible conditions (e.g. mastitis, Mondors syndrome).
7. Other conditions not mentioned above which have similar symptoms, signs and complications as breast cancer and where the differential diagnosis between them and breast cancer is of clinical importance including but not limited to:
   a. Abnormal mammogram and/or nipple retraction and/or nipple discharge due to causes other than breast cancer. Such causes include but are not limited to benign breast masses, melanoma, trauma and technical and/or anatomical variations.
   b. Any condition suggestive of a malignant tumor including but not limited to anorexia, cachexia, weight loss, fever, hypercalcemia, paraneoplastic syndrome.
   c. Lymphadenopathy, weight loss and other signs and symptoms associated with breast cancer but originate from diseases different from breast cancer including but not limited to other malignancies, infections and autoimmune diseases.

### Ovarian Cancer

S57296, HUMGRP5E, T94936, M78530 and/or HSTGFB1 or variants as described herein or markers related thereto are potential markers for ovarian cancer. Other conditions that may be diagnosed by these markers or variants of them include but are not limited to the presence, risk and/or extent of the following:
1. The identification of a metastasis of unknown origin which originated from a primary ovarian cancer, for example gastric carcinoma (such as Krukenberg tumor), breast cancer, colorectal carcinoma and pancreatic carcinoma.
2. Distinguishing between different types of ovarian cancer, therefore potentially affect treatment choice (e.g. discrimination between epithelial tumors and germ cell tumors).
3. Differential diagnosis between a benign and malignant ovarian cysts.
4. Infertility, particularly differential diagnosis of various causes thereof.
5. Other conditions that may elevate serum levels of ovary related markers. These include but are not limited to: cancers of the endometrium, cervix, fallopian tubes, pancreas, breast, lung and colon; nonmalignant conditions such as pregnancy, endometriosis, pelvic inflammatory disease and uterine fibroids.
6. Conditions which have similar symptoms, signs and complications as ovarian cancer and where the differential diagnosis between them and ovarian cancer is of clinical importance including but not limited to:
   a. Non-malignant causes of pelvic mass. Including, but not limited to: benign (functional) ovarian cyst, uterine fibroids, endometriosis, benign ovarian neoplasms and inflammatory bowel lesions
   b. Any condition suggestive of a malignant tumor including but not limited to anorexia, cachexia, weight loss, fever, hypercalcemia, skeletal or abdominal pain, paraneoplastic syndrome.
   c. Ascites.

### Lung Cancer

S57296, HUMGRP5E, T94936, and/or HSTGFB1 or variants as described herein or markers related thereto are potential markers for lung cancer. Other conditions that may be diagnosed by these markers or variants of them include but are not limited to the presence, risk and/or extent of the following:
1. The identification of a metastasis of unknown origin which originated from a primary lung cancer.
2. The assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors.
3. Distinguishing between different types of lung cancer, therefore potentially affect treatment choice (e.g. small cell vs. non small cell tumors).
4. Unexplained dyspnea and/or chronic cough and/or hemoptysis, and analysis thereof.
5. Differential diagnosis of the origin of a pleural effusion.
6. Conditions which have similar symptoms, signs and complications as lung cancer and where the differential diagnosis between them and lung cancer is of clinical importance including but not limited to:
   a. Non-malignant causes of lung symptoms and signs. Symptoms and signs include, but are not limited to: lung lesions and infiltrates, wheeze, stridor.
   b. Other symptoms, signs and complications suggestive of lung cancer, such as tracheal obstruction, esophageal compression, dysphagia, recurrent laryngeal nerve paralysis, hoarseness, phrenic nerve paralysis with elevation of the hemidiaphragm and Horner syndrome.
   c. Any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

### Colorectal Cancer:

Certain splice variants described herein are potential markers for colon cancer. Colon cancer markers according to the present invention which may also optionally have this utility include but are not limited to: S57296, HUMGRP5E, T94936, and/or HSTGFB1 or variants as described herein or markers related thereto. Diagnosis of colon cancer and or of other conditions that may be diagnosed by these markers or variants of them include but are not limited to the presence, risk and/or extent of the following:
1. Early diagnosis, staging, grading, prognosis, monitoring, and treatment of diseases associated with colon cancer, or to indicate a predisposition to such for preventative measures.
2. The identification of a metastasis of unknown origin which originated from a primary colorectal cancer tumor, in particular when the metastasis is located in the liver, lung, bones, supraclavicluar lymph nodes or brain.
3. In the assessment of lymphadenopathy, in particular supraclavicluar or internal abdominal lymphadenopathy.
4. As a marker to distinguish between different types of colorectal tumors including but not limited to nonhereditary carcinoma, Familial Polyposis Coli, Hereditary nonpolyposis colon cancer (Lynch syndrome) and Carcinoid; therefore potentially affect treatment choice.
5. In the assessment of cancer staging, in addition and as a complementary measure to the Dukes system for staging colorectal cancer.
6. As a risk factor to the development of colorectal tumor, and in particular in diseases known to have high incidence of colorectal tumor, including but not limited to Crohn's disease and Ulcerative Colitis.
7. As a tool in the assessment of fecal occult blood or imaging findings suspected for colorectal tumor or abnormal blood tests associated with colorectal cancer including but not limited to elevated CEA level.
8. In the differential diagnosis between malignant and benign colorectal tumors, in particular adenomas and polyps.
9. Other conditions not mentioned above which have similar symptoms, signs and complications as colorectal cancer and where the differential diagnosis between them and colorectal cancer is of clinical importance including but not limited to:
   a. Any condition suggestive of a malignant tumor including but not limited to anorexia, cachexia, weight loss, fever, hypercalcemia, paraneoplastic syndrome.
   b. Lymphadenopathy, weight loss and other signs and symptoms associated with colorectal cancer but originate from diseases different from colorectal cancer including but not limited to other malignancies, infections and autoimmune diseases.
10. Prediction of patient's drug response
11. As surrogate markers for clinical outcome of a treated cancer.

### Related Disease Markers and Risk Factors for Detection by Biomarkers

In addition to the general clinical factors described above, as well as specific diagnostic aspects of each biomarker described below, there are field-specific disease markers/risk factors which may optionally relate to or present diagnostic applications for biomarkers according to the present invention. These field specific factors, as described below, relate to three fields: detection of ovarian cancer (or risk factors thereof), detection of myocardial infarction (or risk factors thereof) and risk factors related to cholesterol which may also serve as diagnostic markers. Each field is described in greater detail below.

### Ovarian Cancer

Known ovarian cancer markers may be used for a variety of diagnoses and/or detection of risk factors, in addition to those related to ovarian cancer itself. These known markers include but are not limited to CA 125. CA 125 may optionally be used for a number of diagnostic assays, such as detection of sepsis (and/or similar bacterial infections) and/or monitoring of the course of infection (as described with regard to PCT Application No. WO 03/048776, hereby incorporated by reference as if fully set forth herein) for example.

Ovarian cancer markers according to the present invention which may also optionally have this utility include but are not limited to: M78530 variants, HUMGRP5E variants, S57296 variants, T94936 variants, and/or HSTGFB1 variants.

### Myocardial Infarction

Known markers for myocardial infarction and/or risk factors thereto may be used for a variety of diagnoses and/or detection of risk factors, in addition to those related to myocardial infarction itself. These known markers include but are not limited to troponin I. Troponin I may optionally be used for determining the time at which a myocardial infarction occurred, as described with regard to U.S. Pat. No. 5,947,124, hereby incorporated by reference as if fully set forth herein. The method optionally and preferably involves measuring the ratio of oxidized to reduced troponin I in a blood sample obtained from the patient. The measured ratio reflects the time elapsed from the time of the myocardial infarction.

Another optional utility involves diagnosing the presence of congestive heart failure and preferably predicting mortality of a subject suffering from congestive heart failure, by detecting troponin I in a sample taken from the subject (as described with regard to US Patent Application No. 2004/0096989, hereby incorporated by reference as if fully set forth herein).

Markers according to the present invention which may also optionally have these utilities include but are not limited to: N56180 variants, S67314 variants, HUMNATPEP variants, HUMCDDANF variants, HSACMHCP variants, HSCREACT variants and/or Z3624 variants.

### Cholesterol

Abnormal cholesterol profile is a known risk factor for a number of diseases and conditions, including but not limited to cardiac diseases (both acute and chronic), atherosclerosis in general, stroke, metabolic syndrome and Alzheimer's disease (for a description of the relationship between high cholesterol levels and Alzheimer's disease, see for example Yanagisawa, Subcell Biochem. 2005; 38:179-202). Abnormal cholesterol profiles can also combine with other diseases and conditions as risk factors for yet other diseases and conditions. One example of such a combination is the association of high cholesterol levels and metabolic syndrome with increased risk for stroke (see for example Brown, Clin Cornerstone. 2004; 6 Suppl 3:S30-4).

Cardiac diseases that are affected by an abnormal cholesterol profile include all of the cardiovascular diseases described previously, plus arterial stiffness, atherosclerosis and peripheral vascular disease. In addition to diagnosis of such diseases or a tendency thereto, abnormal cholesterol profiles may optionally be used to detect a tendency toward other diseases for which arterial stiffness, atherosclerosis and peripheral vascular disease are early warning signs, including but not limited to stroke and circulation-related peripheral tissue damage, such as skin ulcers for example. The latter are quite frequent in diabetics and can result in significant damage, including loss of limbs through amputation.

PCT Application No. WO 02/062300, hereby incorporated by reference as if fully set forth herein, describes the link between cholesterol levels and a number of cognitive or psychological disorders, including but not limited to, age-related memory loss, mild cognitive impairment, dementia, substance abuse disorders (including but not limited to disorders characterized by an abuse of or dependence on a substance selected from the group consisting of alcohol, stimulants, opiates, marijuana, solvents, and nicotine), depression, dysthymia, cyclothymia, bipolar disorder, schizoaffective disorder, and borderline personality disorder.

Markers according to the present invention which may also optionally have these utilities include but are not limited to: N56180 variants, S67314 variants, HUMNATPEP variants, HUMCDDANF variants, HSACMHCP variants, HSCREACT variants and/or Z3624 variants.

### Candidate Marker Examples Section

This section relates to examples of sequences according to the present invention, including illustrative methods of selection thereof.

The markers of the present invention were tested with regard to their expression in various cancerous and non-cancerous tissue samples. A description of the samples used in the prostate cancer testing panel is provided in Table 2 below. A description of the samples used in the ovarian cancer testing panel is provided in Table 3 below. A description of the samples used in the colon cancer testing panel is provided in Table 4 below. A description of the samples used in the lung cancer testing panel is provided in Table 5 below. A description of the samples used in the breast cancer testing panel is provided in Table 6 below. A description of the samples used in the normal tissue panel, used also for the testing of the markers of the present invention with regard to their expression in various heart and non-heart tissue samples, is provided in Table 7 below. Tests were then performed as described in the “Materials and Experimental Procedures” section below.

TABLE 2
Tissue samples in prostate cancer testing panel

| | Lot No. | Pathology | Sex/Age | Source |
|---|---|---|---|---|
| 66-A-Adeno G1 GS-4 | 160202 | Adenocarcinoma Gleason score 4 | M/64 | ABS |
| 73-A-Adeno G1 GS-4 | 16026T2 | Acinar Adenocarcinoma | M/77 | ABS |
| | | Gleason score 4 (2 + 2) | | |
| 68-A-Adeno G1 GS-5 | 160172 | Adenocarcinoma Gleason score 5 | M/66 | ABS |
| 56-Am-Adeno G1 GS-5 | 36467 | Adenocarcinoma, Gleason | M/72 | Ambion |
| | | score 5 (3 + 2); stage 2 | | |
| 58-Am-Adeno G1 GS-5 | 37192 | Adenocarcinoma, Gleason | M/52 | Ambion |
| | | score 5; stage 2 | | |
| 65-A-Adeno G2 GS-5 | 160022 | Adenocarcinoma Gleason score 5 | M/66 | ABS |
| 69-A-Adeno GS-5 | 160182 | Acinar Adenocarcinoma | M/58 | ABS |
| | | Gleason score 5 | | |
| 55-Am-Adeno GS-5 | 36464 | Adenocarcinoma, Gleason | M/53 | Ambion |
| | | score 5; stage 1 | | |
| 64-A-Adeno G2 GS-6 | 160092 | Acinar Adenocarcinoma | M/71 | ABS |
| | | Gleason score 6 | | |
| 70-A-Adeno G2 GS-6 | 160192 | Adenocarcinoma Gleason score 6 | M/53 | ABS |
| 18-A-Adeno GS-6 | 5610020069T | Adenocarcinoma, Gleason | M | ABS |
| | | score 6 (3 + 3) | | |
| 67-A-Adeno GS-6 | 160142 | Acinar Adenocarcinoma | M/62 | ABS |
| | | Gleason score 6 | | |
| 25-A-Adeno GS-7 | 5605020052T | Adenocarcinoma, Gleason | M | ABS |
| | | score 7 (4 + 3) | | |
| 26-A-Adeno GS-7 | 5609020067T | Adenocarcinoma, Gleason | M | ABS |
| | | score 7 (4 + 3) | | |
| 72-A-Adeno GS-7 | 160122 | Acinar Adenocarcinoma | M/66 | ABS |
| | | Gleason score 7 | | |
| 71-A-Adeno GS-7 | 160242 | Acinar Adenocarcinoma | M/70 | ABS |
| | | Gleason score 7 | | |
| 57-Am-Adeno GS-7 | 26442 | Adenocarcinoma, Gleason | M/62 | Ambion |
| | | score 7 | | |
| 32-A-Adeno GS-9 | 5604020042T | Adenocarcinoma, Gleason | M | ABS |
| | | score 9 (5 + 4) | | |
| 54-B-Adeno G3 | A610031 | Adenocarcinoma | | Biochain |
| 33-A-BPH | 5607020058 | BPH | M | ABS |
| 34-A-BPH | 5607020059 | BPH | M | ABS |
| 35-A-BPH | 5607020060 | BPH | M | ABS |
| 43-B-PBH | A609267 | BPH | M/66 | Biochain |
| 44-B-PBH | A609268 | BPH | M/72 | Biochain |
| 45-B-PBH | A609269 | BPH | M/69 | Biochain |
| 46-B-PBH | A609270 | BPH | M/65 | Biochain |
| 47-B-PBH | A609271 | BPH | M/71 | Biochain |
| 40-A-N M26 | 5609020067N | Normal Matched | M | ABS |
| 41-A-N M32 | 5604020042N | Normal Matched | M | ABS |
| 48-B-N | A609257 | Normal PM | M/24 | Biochain |
| 49-B-N | A609256 | Normal PM | M/36 | Biochain |
| 50-B-N | A609255 | Normal PM | M/26 | Biochain |
| 51-B-N | A609258 | Normal PM | M/27 | Biochain |
| 52-B-N | A609254 | Normal PM | M/29 | Biochain |
| 53-Cl-N | 1070317 | Normal - Pool of 47 | M&F | Clontech |
| 42-Am-N | 061P04A | Normal (IC BLEED) | M/47 | ambion |
| 59-Am-N | 25955 | Normal PM (Head trauma) | M/62 | Ambion |
| 60-Am-N | 33605 | Normal PM (Myocardial | M/69 | Ambion |
| | | infraction) | | |
| 61-Am-N | 34077 | Normal PM (Alzheimer's) | M/71 | Ambion |
| 62-Am-N | 31316 | Normal (Renal failure) | M/79 | Ambion |
| 63-Am-N | 30991 | Normal (Gall Bladder cancer) | M/78 | Ambion |

TABLE 3
Tissue samples in ovarian cancer testing panel

| Sample name | Lot number | Source | Pathology | Grade | Age |
|---|---|---|---|---|---|
| 33-B-Pap Sero CystAde G1 | A503175 | BioChain | Serous papillary | 1 | 41 |
| | | | cystadenocarcinoma | | |
| 41-G-Mix Sero/Muc/Endo G2 | 98-03-G803 | GOG | Mixed epithelial | 2 | 38 |
| | | | cystadenocarcinoma with | | |
| | | | mucinous, endometrioid, | | |
| | | | squamous and papillary serous | | |
| | | | (Stage 2) | | |
| 35-G-Endo Adeno G2 | 94-08-7604 | GOG | Endometrioid adenocarcinoma | 2 | 39 |
| 14-B-Adeno G2 | A501111 | BioChain | Adenocarcinoma | 2 | 41 |
| 12-B-Adeno G3 | A406023 | Biochain | Adenocarcinoma | 3 | 45 |
| 40-G-Mix Sero/Endo G2 | 95-11-G006 | GOG | Papillary serous and endometrioid | 2 | 49 |
| | | | cystadenocarcinoma (Stage 3C) | | |
| 4-A-Pap CystAdeno G2 | ILS-7286 | ABS | Papillary cystadenocarcinoma | 2 | 50 |
| 3-A-Pap Adeno G2 | ILS-1431 | ABS | Papillary adenocarcinoma | 2 | 52 |
| 2-A-Pap Adeno G2 | ILS-1408 | ABS | Papillary adenocarcinoma | 2 | 53 |
| 5-G-Adeno G3 | 99-12-G432 | GOG | Adenocarcinoma (Stage 3C) | 3 | 46 |
| 11-B-Adeno G3 | A407068 | Biochain | Adenocarcinoma | 3 | 49 |
| 39--G-Mix Sero/Endo G3 | 2001-12-G037 | GOG | Mixed serous and endometrioid | 3 | 49 |
| | | | adenocarcinoma | | |
| 29-G-Sero Adeno G3 | 2001-12-G035 | GOG | Serous adenocarcinoma (Stage 3A) | 3 | 50 |
| 70-G-Pap Sero Adeno G3 | 95-08-G069 | GOG | Papillary serous adenocarcinoma | 3 | 50 |
| 6-A-Adeno G3 | A0106 | ABS | adenocarcinoma | 3 | 51 |
| 31-B-Pap Sero CystAde G3 | A503176 | BioChain | Serous papillary | 3 | 52 |
| | | | cystadenocarcinoma | | |
| 25-A-Pap Sero Adeno G3 | N0021 | ABS | Papillary serous adenocarcinoma | 3 | 55 |
| | | | (Stage T3CN1MX) | | |
| 37-G-Mix Sero/Endo G3 | 2002-05-G513 | GOG | Mixed serous and endometrioid | 3 | 56 |
| | | | adenocarcinoma | | |
| 7-A-Adeno G3 | IND-00375 | ABS | adenocarcinoma | 3 | 59 |
| 8-B-Adeno G3 | A501113 | BioChain | adenocarcinoma | 3 | 60 |
| 10-B-Adeno G3 | A407069 | Biochain | Adenocarcinoma | 3 | 60 |
| 38-G-Mix Sero/Endo G3 | 2002-05-G509 | GOG | Mixed serous and endometrioid | 3 | 64 |
| | | | adenocarcinoma of mullerian | | |
| | | | (Stage 3C) | | |
| 13-G-Adeno G3 | 94-05-7603 | GOG | Poorly differentiated | 3 | 67 |
| | | | adenocarcinoma from primary | | |
| | | | peritoneal | | |
| 24-G-Pap Sero Adeno G3 | 2001-07-G801 | GOG | Papillary serous adenocarcinoma | 3 | 68 |
| 34-G-Pap Endo Adeno G3 | 95-04-2002 | GOG | Papillary endometrioid | 3 | 68 |
| | | | adenocarcinoma (Stage 3C) | | |
| 30-G-Pap Sero Adeno G3 | 2001-08-G011 | GOG | Papillary serous carcinoma | 3 | 72 |
| | | | (Stage 1C) | | |
| 1-A-Pap Adeno G3 | ILS-1406 | ABS | Papillary adenocarcinoma | 3 | 73 |
| 9-G-Adeno G3 | 99-06-G901 | GOG | Adenocarcinoma (maybe serous) | 3 | 84 |
| 32-G-Pap Sero CystAde G3 | 93-09-4901 | GOG | Serous papillary | 3 | 67 |
| | | | cystadenocarcinoma | | |
| 66-G-Pap Sero Adeno G3 SIV | 2000-01-G413 | GOG | Papillary serous carcinoma | 3 | 67 |
| | | | (metastais of primary peritoneum) | | |
| | | | (Stage 4) | | |
| 19-B-Muc Adeno G3 | A504085 | BioChain | Mucinous adenocarcinoma | 3 | 34 |
| 21-G-Muc CystAde G2-3 | 95-10-G020 | GOG | Mucinous cystadenocarcinoma | 2-3 | 44 |
| | | | (Stage 2) | | |
| 18-B-Muc Adeno G3 | A504083 | BioChain | Mucinous adenocarcinoma | 3 | 45 |
| 20-A-Pap Muc CystAde | USA-00273 | ABS | Papillary mucinous | | 46 |
| | | | cystadenocarcinoma | | |
| 17-B-Muc Adeno G3 | A504084 | BioChain | Mucinous adenocarcinoma | 3 | 51 |
| 22-A-Muc CystAde G2 | A0139 | ABS | Mucinous cystadenocarcinoma | 2 | 72 |
| | | | (Stage 1C) | | |
| 43-G-Clear cell Adeno G3 | 2001-10-G002 | GOG | Clear cell adenocarcinoma | 3 | 74 |
| 44-G-Clear cell Adeno | 2001-07-G084 | GOG | Clear cell adenocarcinoma | | 73 |
| | | | (Stage 3A) | | |
| 15-B-Adeno G3 | A407065 | BioChain | Carcinoma | 3 | 27 |
| 16-Ct-Adeno | 1090387 | Clontech | Carcinoma NOS | NA | 58 |
| 23-A-Muc CystAde G3 | VNM-00187 | ABS | Mucinous cystadenocarcinoma | 3 | 45 |
| | | | with low malignant | | |
| 42-G-Adeno borderline | 98-08-G001 | GOG | Epithelial adenocarcinoma of | | 46 |
| | | | borderline malignancy | | |
| 63-G-Sero CysAdenoFibroma | 2000-10-G620 | GOG | Serous CysAdenoFibroma of | | 71 |
| | | | borderline malignancy | | |
| 62-G-Ben Muc CysAdenoma | 99-10-G442 | GOG | Benbin mucinus cysadenoma | | 32 |
| 60-G-Muc CysAdenoma | 99-01-G043 | GOG | Mucinous Cysadenoma | | 40 |
| 56-G-Ben Muc CysAdeno | 99-01-G407 | GOG | Bengin mucinus cysadenoma | | 46 |
| 64-G-Ben Sero CysAdenoma | 99-06-G039 | GOG | Bengin Serous CysAdenoma | | 57 |
| 61-G-Muc CysAdenoma | 99-07-G011 | GOG | Mucinous Cysadenoma | | 63 |
| 59-G-Sero CysAdenoFibroma | 98-12-G401 | GOG | Serous CysAdenoFibroma | | 77 |
| 51-G-N M41 | 98-03-G803N | GOG | Normal (matched tumor 98-03- | | 38 |
| | | | G803) | | |
| 75-G-N M60 | 99-01-G043N | GOG | Normal (matched tumor 99-01- | | 40 |
| | | | G043) | | |
| 49-B-N M14 | A501112 | BioChain | Normal (matched tumor | | 41 |
| | | | A501111) | | |
| 52-G-N M42 | 98-08-G001N | GOG | Normal (matched tumor 98-08- | | 46 |
| | | | G001) | | |
| 68-G-N M56 | 99-01-G407N | GOG | Normal (matched bengin 99-01- | | 46 |
| | | | G407) | | |
| 50-B-N M8 | A501114 | BioChain | Normal (matched tumor | | 60 |
| | | | A501113) | | |
| 67-G-N M38 | 2002-05-509N | GOG | Normal (matched tumor 2002-05- | | 64 |
| | | | G509) | | |
| 69-G-N M24 | 2001-07-G801N | GOG | Normal (matched tumor 2001-07- | | 68 |
| | | | G801) | | |
| 73-G-N M59 | 98-12-G401N | GOG | Normal (matched tumor 98-12- | | 77 |
| | | | G401) | | |
| 72-G-N M66 | 2000-01-G413N | GOG | Normal (matched tumor 2000-01- | | |
| | | | G413) | | |
| 45-B-N | A503274 | BioChain | Normal PM | | 41 |
| 46-B-N | A504086 | BioChain | Normal PM | | 41 |
| 71-CG-N | CG-188-7 | Ichilov | Normal PM | | 49 |
| 48-B-N | A504087 | BioChain | Normal PM | | 51 |

TABLE 4
Tissue samples in colon cancer testing panel

| COLON PANEL | | | | | | gender/ |
|---|---|---|---|---|---|---|
| sample name | Lot No. | tissue | source | pathology | Grade | age |
| 58-B-Adeno G1 | A609152 | Colon | biochain | Adenocarcinoma | 1 | M/73 |
| 59-B-Adeno G1 | A609059 | Colon | biochain | Adenocarcinoma, Ulcer | 1 | M/58 |
| 14-CG-Polypoid Adeno | CG-222 (2) | Rectum | Ichilov | Well polypoid adeocarcinoma Duke's C | | F/49 |
| G1 D-C | | | | | | |
| 17-CG-Adeno G1-2 | CG-163 | Rectum | Ichilov | Adenocarcinoma | 2 | M/73 |
| 10-CG-Adeno G1-2 D-B2 | CG-311 | Sigmod co | Ichilov | Adenocarcinoma Astler-Coller B2. | 1-2 | M/88 |
| 11-CG-Adeno G1-2 D-C2 | CG-337 | Colon | Ichilov | Adenocarcinoma Astler-Coller C2. | 1-2 | NA |
| 6-CG-Adeno G1-2 D-C2 | CG-303 (3) | Colon | Ichilov | Adenocarcinoma Astler-Coller C2. | 1-2 | F/77 |
| 5-CG-Adeno G2 | CG-308 | Colon Sign | Ichilov | Adenocarcinoma. | 2 | F/80 |
| 16-CG-Adeno G2 | CG-278C | colon | Ichilov | Adenocarcinoma | 2 | F/60 |
| 56-B-Adeno G2 | A609148 | Colon | biochain | Adenocarcinoma | 2 | F48 |
| 61-B-Adeno G2 | A606258 | Colon | biochain | Adenocarcinoma, Ulcer | 2 | M/41 |
| 60-B-Adeno G2 | A609058 | Colon | biochain | Adenocarcinoma, Ulcer | 2 | M/67 |
| 22-CG-Adeno G2 D-B | CG-229C | Colon | Ichilov | Adenocarcinoma Duke's B | 2 | F/55 |
| 1-CG-Adeno G2 D-B2 | CG-335 | Cecum | Ichilov | Adenocarcinoma Dukes B2. | 2 | F/66 |
| 12-CG-Adeno G2 D-B2 | CG-340 | Colon Sign | Ichilov | Adenocarcinoma Astler-Coller B2. | 2 | M/66 |
| 28-CG-Adeno G2 D-B2 | CG-284 | sigma | Ichilov | Adenocarcinoma Duke's B2 | 2 | F/72 |
| 2-CG-Adeno G2 D-C2 | CG-307 X2 | Cecum | Ichilov | Adenocarcinoma Astler-Coller C2. | 2 | F/89 |
| 9-CG-Adeno G2 D-D | CG-297 X2 | Rectum | Ichilov | Adenocarcinoma Dukes D. | 2 | M/62 |
| 13-CG-Adeno G2 D-D | CG-290 X2 | Rectosigm | Ichilov | Adenocarcinoma Dukes D. | 2 | M/47 |
| 26-CG-Adeno G2 D-D | CG-283 | sigma | Ichilov | Colonic adenocarcinoma Duke's D | 2 | F/63 |
| 4-CG-Adeno G3 | CG-276 | Colon | Ichilov | Carcinoma. | 3 | M/64 |
| 53-B-Adeno G3 | A609161 | Colon | biochain | Adenocarcinoma | 3 | F/53 |
| 54-B-Adeno G3 | A609142 | Colon | biochain | Adenocarcinoma | 3 | M/53 |
| 55-B-Adeno G3 | A609144 | Colon | biochain | Adenocarcinoma | 3 | M/68 |
| 57-B-Adeno G3 | A609150 | Colon | biochain | Adenocarcinoma | 3 | F/45 |
| 72-CG-Adeno G3 | CG-309 | colon | Ichilov | Adenocarcinoma | 3 | F/88 |
| 20-CG-Adeno G3 D-B2 | CG-249 | Colon | Ichilov | Ulcerated adenocarcinoma Duke's B2 | 3 | M/36 |
| 7-CG-Adeno D-A | CG-235 | Rectum | Ichilov | Adenocarcinoma intramucosal Duke's A. | | F/66 |
| 23-CG-Adeno D-C | CG-282 | sigma | Ichilov | Mucinus adenocarcinoma Astler Coller C | | M/51 |
| 3-CG-Muc adeno D-D | CG-224 | Colon | Ichilov | Mucinois adenocarcinoma Duke's D | | M/48 |
| 18-CG-Adeno | CG-22C | Colon | Ichilov | Adenocarcinoma | | NA |
| 19-CG-Adeno | CG-19C (1) | Colon | Ichilov | Adenocarcinoma | | NA |
| 21-CG-Adeno | CG-18C | Colon | Ichilov | Adenocarcinoma | | NA |
| 24-CG-Adeno | CG-12 (2) | Colon | Ichilov | Adenocarcinoma | | NA |
| 25-CG-Adeno | CG-2 | Colon | Ichilov | Adenocarcinoma | | NA |
| 27-CG-Adeno | CG-4 | Colon | Ichilov | Adenocarcinoma | | NA |
| 8-CG-diverticolosis, | CG-291 | Wall of sig [symbol] | Ichilov | Diverticolosis and diverticulitis of the Colon | | F/65 |
| diverticulitis | | | | | | |
| 46-CG-Crohn's disease | CG-338C | Cecum | Ichilov | Crohn's disease | | M/22 |
| 47-CG-Crohn's disease | CG-338AC | Colon | Ichilov | Crohn's disease. | | M/22 |
| 42-CG-N M20 | CG-249N | Colon | Ichilov | Normal | | M/36 |
| 43-CG-N M8 | CG-291N | Wall of sig [symbol] | Ichilov | Normal | | F/65 |
| 44-CG-N M21 | CG-18N | Colon | Ichilov | Normal | | NA |
| 45-CG-N M11 | CG-337N | Colon | Ichilov | Normal | | M/75 |
| 49-CG-N M14 | CG-222N | Rectum | Ichilov | Normal | | F/49 |
| 50-CG-N M5 | CG-308N | Sigma | Ichilov | Within normal limits | | F/80 |
| 51-CG-N M26 | CG-283N | Sigma | Ichilov | Normal | | F/63 |
| 41-B-N | A501156 | Colon | biochain | Normal PM | | M/78 |
| 52-CG-N | CG-309TR | Colon | Ichilov | Within normal limits | | F/88 |
| 62-B-N | A608273 | Colon | biochain | Normal PM | | M/66 |
| 63-B-N | A609260 | Colon | biochain | Normal PM | | M/61 |
| 64-B-N | A609261 | Colon | biochain | Normal PM | | F/68 |
| 65-B-N | A607115 | Colon | biochain | Normal PM | | M/24 |
| 66-B-N | A609262 | Colon | biochain | Normal PM | | M/58 |
| 67-B-N | A406029 | Colon | biochain | Normal PM (Pool of 10) | | |
| 69-B-N | A411078 | Colon | biochain | Normal PM (Pool of 10) | | F&M |
| 70-Cl-N | 1110101 | Colon | clontech | Normal PM (Pool of 3) | | |
| 71-Am-N | 071P10B | Colon | Ambion | Normal (IC BLEED) | | F/34 |

[symbol]

indicates data missing or illegible when filed

TABLE 5
Tissue samples in lung cancer testing panel

| sample rename | Lot No. | source | pathology | Grade | gender/age |
|---|---|---|---|---|---|
| 1-B-Adeno G1 | A504117 | Biochain | Adenocarcinoma | 1 | F/29 |
| 2-B-Adeno G1 | A504118 | Biochain | Adenocarcinoma | 1 | M/64 |
| 95-B-Adeno G1 | A610063 | Biochain | Adenocarcinoma | 1 | F/54 |
| 12-B-Adeno G2 | A504119 | Biochain | Adenocarcinoma | 2 | F/74 |
| 75-B-Adeno G2 | A609217 | Biochain | Adenocarcinoma | 2 | M/65 |
| 77-B-Adeno G2 | A608301 | Biochain | Adenocarcinoma | 2 | M/44 |
| 13-B-Adeno G2-3 | A504116 | Biochain | Adenocarcinoma | 2-3 | M/64 |
| 89-B-Adeno G2-3 | A609077 | Biochain | Adenocarcinoma | 2-3 | M/62 |
| 76-B-Adeno G3 | A609218 | Biochain | Adenocarcinoma | 3 | M/57 |
| 94-B-Adeno G3 | A610118 | Biochain | Adenocarcinoma | 3 | M/68 |
| 3-CG-Adeno | CG-200 | Ichilov | Adenocarcinoma | | NA |
| 14-CG-Adeno | CG-111 | Ichilov | Adenocarcinoma | | M/68 |
| 15-CG-Bronch adeno | CG-244 | Ichilov | Bronchioloalveolar | | M/74 |
| | | | adenocarcinoma | | |
| 45-B-Alvelous Adeno | A501221 | Biochain | Alveolus | | F/50 |
| | | | carcinoma | | |
| 44-B-Alvelous Adeno G2 | A501123 | Biochain | Alveolus | 2 | F/61 |
| | | | carcinoma | | |
| 19-B-Squamous G1 | A408175 | Biochain | Squamous | 1 | M/78 |
| | | | carcinoma | | |
| 16-B-Squamous G2 | A409091 | Biochain | Squamous | 2 | F/68 |
| | | | carcinoma | | |
| 17-B-Squamous G2 | A503183 | Biochain | Squamous | 2 | M/57 |
| | | | carcinoma | | |
| 21-B-Squamous G2 | A503187 | Biochain | Squamous | 2 | M/52 |
| | | | carcinoma | | |
| 78-B-Squamous G2 | A607125 | Biochain | Squamous Cell | 2 | M/62 |
| | | | Carcinoma | | |
| 80-B-Squamous G2 | A609163 | Biochain | Squamous Cell | 2 | M/74 |
| | | | Carcinoma | | |
| 18-B-Squamous G2-3 | A503387 | Biochain | Squamous Cell | 2-3 | M/63 |
| | | | Carcinoma | | |
| 81-B-Squamous G3 | A609076 | Biochain | Squamous | 3 | m/53 |
| | | | Carcinoma | | |
| 79-B-Squamous G3 | A609018 | Biochain | Squamous Cell | 3 | M/67 |
| | | | Carcinoma | | |
| 20-B-Squamous | A501121 | Biochain | Squamous | | M/64 |
| | | | Carcinoma | | |
| 22-B-Squamous | A503386 | Biochain | Squamous | | M/48 |
| | | | Carcinoma | | |
| 88-B-Squamous | A609219 | Biochain | Squamous Cell | | M/64 |
| | | | Carcinoma | | |
| 100-B-Squamous | A409017 | Biochain | Squamous | | M/64 |
| | | | Carcinoma | | |
| 23-CG-Squamous | CG-109 (1) | Ichilov | Squamous | | M/65 |
| | | | Carcinoma | | |
| 24-CG-Squamous | CG-123 | Ichilov | Squamous | | M/76 |
| | | | Carcinoma | | |
| 25-CG-Squamous | CG-204 | Ichilov | Squamous | | M/72 |
| | | | Carcinoma | | |
| 87-B-Large cell G3 | A609165 | Biochain | Large Cell | 3 | F/47 |
| | | | Carcinoma | | |
| 38-B-Large cell | A504113 | Biochain | Large cell | | M/58 |
| 39-B-Large cell | A504114 | Biochain | Large cell | | F/35 |
| 82-B-Large cell | A609170 | Biochain | Large Cell | | M/68 |
| | | | Neuroendocrine | | |
| | | | Carcinoma | | |
| 30-B-Small cell carci G3 | A501389 | Biochain | small cell | 3 | M/34 |
| 31-B-Small cell carci G3 | A501390 | Biochain | small cell | 3 | F/59 |
| 32-B-Small cell carci G3 | A501391 | Biochain | small cell | 3 | M/30 |
| 33-B-Small cell carci G3 | A504115 | Biochain | small cell | 3 | M |
| 86-B-Small cell carci G3 | A608032 | Biochain | Small Cell | 3 | F/52 |
| | | | Carcinoma | | |
| 83-B-Small cell carci | A609162 | Biochain | Small Cell | | F/47 |
| | | | Carcinoma | | |
| 84-B-Small cell carci | A609167 | Biochain | Small Cell | | F/59 |
| | | | Carcinoma | | |
| 85-B-Small cell carci | A609169 | Biochain | Small Cell | | M/66 |
| | | | Carcinoma | | |
| 46-B-N M44 | A501124 | Biochain | Normal M44 | | F/61 |
| 47-B-N | A503205 | Biochain | Normal PM | | M/26 |
| 48-B-N | A503206 | Biochain | Normal PM | | M/44 |
| 49-B-N | A503384 | Biochain | Normal PM | | M/27 |
| 50-B-N | A503385 | Biochain | Normal PM | | M/28 |
| 90-B-N | A608152 | Biochain | Normal (Pool 2) | | pool 2 |
| | | | PM | | |
| 91-B-N | A607257 | Biochain | Normal (Pool 2) | | pool 2 |
| | | | PM | | |
| 92-B-N | A503204 | Biochain | Normal PM | | m/28 |
| 93-Am-N | 111P0103A | Ambion | Normal PM | | F/61 |
| 96-Am-N | 36853 | Ambion | Normal PM | | F/43 |
| 97-Am-N | 36854 | Ambion | Normal PM | | M/46 |
| 98-Am-N | 36855 | Ambion | Normal PM | | F/72 |
| 99-Am-N | 36856 | Ambion | Normal PM | | M/31 |

TABLE 6
Tissue samples in breast cancer testing panel

| sample rename | Lot no | source | pathology | grade | age | TNM | stage |
|---|---|---|---|---|---|---|---|
| 14-A-IDC G2 | A0135T | ABS | IDC | 2 | 37 | T2N2Mx | |
| 43-B-IDC G2 | A609183 | Biochain | IDC | 2 | 40 | | |
| 54-B-IDC G2 | A605353 | Biochain | IDC | 2 | 41 | | |
| 55-B-IDC G2 | A609179 | Biochain | IDC | 2 | 42 | | |
| 47-B-IDC G2 | A609221 | Biochain | IDC | 2 | 42 | | |
| 17-A-IDC G2 | 4904020036T | ABS | IDC | 2-3 | 42 | T3N1Mx | |
| 42-A-IDC G3 | 6005020031T | ABS | IDC | 3 | 42 | T1cN0Mx | |
| 7-A-IDC G2 | 7263T | ABS | IDC | 2 | 43 | T1N0M0 | stage 1 |
| 48-B-IDC G2 | A609222 | Biochain | IDC | 2 | 44 | | |
| 53-B-IDC G2 | A605151 | Biochain | IDC | 2 | 44 | | |
| 12-A-IDC G2 | 1432T | ABS | IDC | 2 | 46 | T2N0M0 | stage 2A |
| 61-B-IDC G2 | A610029 | Biochain | IDC | 2 | 46 | | |
| 46-B-Carci G2 | A609177 | Biochain | Carcinoma | 2 | 48 | | |
| 16-A-IDC G2 | 4904020032T | ABS | IDC | 2 | 49 | T3N1Mx | |
| 62-B-IDC G2 | A609194 | Biochain | IDC | 2 | 51 | | |
| 49-B-IDC G2 | A609223 | Biochain | IDC | 2 | 54 | | |
| 32-A-Muc Carci | 7116T | ABS | Mucinous | | 54 | T2N0M0 | stage 2A |
| | | | carcinoma | | | | |
| 45-B-IDC G2 | A609181 | Biochain | IDC | 2 | 58 | | |
| 15-A-IDC G2 | 7259T | ABS | IDC | 2 | 59 | T3N1M0 | stage 3A |
| 52-B-ILC G1 | A605360 | Biochain | Invasive | 1 | 60 | | |
| | | | Lobular | | | | |
| | | | Carcinoma | | | | |
| 6-A-IDC G1 | 7238T | ABS | IDC | 1 | 60 | T2N0M0 | stage 2A |
| 26-A-IDC G3 | 7249T | ABS | IDC | 3 | 60 | T2N0M0 | stage 2A |
| 13-A-IDC G2 | A0133T | ABS | IDC | 2 | 63 | T2N1aMx | |
| 50-B-IDC G2 | A609224 | Biochain | IDC | 2 | 69 | | |
| 44-B-IDC G2 | A609198 | Biochain | IDC | 2 | 77 | | |
| 51-B-IDC G1 | A605361 | Biochain | IDC | 1 | 79 | | |
| 31-CG-IDC | CG-154 | Ichilov | IDC | | 83 | | |
| 27-A-IDC G3 | 4907020072T | ABS | IDC | 3 | 91 | T2N0Mx | |
| 36-A-N M7 | 7263N | ABS | Normal | | 43 | | |
| | | | matched to | | | | |
| | | | 7T | | | | |
| 40-A-N M12 | 1432N | ABS | Normal | | 46 | | |
| | | | matched to | | | | |
| | | | 12T | | | | |
| 39-A-N M15 | 7259N | ABS | Normal | | 59 | | |
| | | | matched to | | | | |
| | | | 15T | | | | |
| 35-A-N M6 | 7238N | ABS | Normal | | 60 | | |
| | | | matched to | | | | |
| | | | 6T | | | | |
| 41-A-N M26 | 7249N | ABS | Normal | | 60 | | |
| | | | matched to | | | | |
| | | | 26T | | | | |
| 57-B-N | A609233 | Biochain | Normal PM | | 34 | | |
| 59-B-N | A607155 | Biochain | Normal PM | | 35 | | |
| 60-B-N | A609234 | Biochain | Normal PM | | 36 | | |
| 63-Am-N | 26486 | Ambion | Normal PS | | 43 | | |
| 66-Am-N | 36678 | Ambion | Normal PM | | 45 | | |
| 64-Am-N | 23036 | Ambion | Normal PM | | 57 | | |
| 56-B-N | A609235 | Biochain | Normal PM | | 59 | | |
| 65-Am-N | 31410 | Ambion | Normal PM | | 63 | | |
| 67-Am-N | 073P010602086A | Ambion | Normal PM | | 64 | | |
| 58-B-N | A609232 | Biochain | Normal PM | | 65 | | |

TABLE 7
Tissue samples in normal panel:

| | Lot no. | Source | Tissue | Pathology | Sex/Age |
|---|---|---|---|---|---|
| 1-Am-Colon (C71) | 071P10B | Ambion | Colon | PM | F/43 |
| 2-B-Colon (C69) | A411078 | Biochain | Colon | PM-Pool of 10 | M&F |
| 3-Cl-Colon (C70) | 1110101 | Clontech | Colon | PM-Pool of 3 | M&F |
| 4-Am-Small Intestine | 091P0201A | Ambion | Small Intestine | PM | M/75 |
| 5-B-Small Intestine | A501158 | Biochain | Small Intestine | PM | M/63 |
| 6-B-Rectum | A605138 | Biochain | Rectum | PM | M/25 |
| 7-B-Rectum | A610297 | Biochain | Rectum | PM | M/24 |
| 8-B-Rectum | A610298 | Biochain | Rectum | PM | M/27 |
| 9-Am-Stomach | 110P04A | Ambion | Stomach | PM | M/16 |
| 10-B-Stomach | A501159 | Biochain | Stomach | PM | M/24 |
| 11-B-Esophagus | A603814 | Biochain | Esophagus | PM | M/26 |
| 12-B-Esophagus | A603813 | Biochain | Esophagus | PM | M/41 |
| 13-Am-Pancreas | 071P25C | Ambion | Pancreas | PM | M/25 |
| 14-CG-Pancreas | CG-255-2 | Ichilov | Pancreas | PM | M/75 |
| 15-B-Lung | A409363 | Biochain | Lung | PM | F/26 |
| 16-Am-Lung (L93) | 111P0103A | Ambion | Lung | PM | F/61 |
| 17-B-Lung (L92) | A503204 | Biochain | Lung | PM | M/28 |
| 18-Am-Ovary (O47) | 061P43A | Ambion | Ovary | PM | F/16 |
| 19-B-Ovary (O48) | A504087 | Biochain | Ovary | PM | F/51 |
| 20-B-Ovary (O46) | A504086 | Biochain | Ovary | PM | F/41 |
| 21-Am-Cervix | 101P0101A | Ambion | Cervix | PM | F/40 |
| 22-B-Cervix | A408211 | Biochain | Cervix | PM | F/36 |
| 23-B-Cervix | A504089 | Biochain | Cervix | PM-Pool of 5 | M&F |
| 24-B-Uterus | A411074 | Biochain | Uterus | PM-Pool of 10 | M&F |
| 25-B-Uterus | A409248 | Biochain | Uterus | PM | F/43 |
| 26-B-Uterus | A504090 | Biochain | Uterus | PM-Pool of 5 | M&F |
| 27-B-Bladder | A501157 | Biochain | Bladder | PM | M/29 |
| 28-Am-Bladder | 071P02C | Ambion | Bladder | PM | M/20 |
| 29-B-Bladder | A504088 | Biochain | Bladder | PM-Pool of 5 | M&F |
| 30-Am-Placenta | 021P33A | Ambion | Placenta | PB | F/33 |
| 31-B-Placenta | A410165 | Biochain | Placenta | PB | F/26 |
| 32-B-Placenta | A411073 | Biochain | Placenta | PB-Pool of 5 | M&F |
| 33-B-Breast (B59) | A607155 | Biochain | Breast | PM | F/36 |
| 34-Am-Breast (B63) | 26486 | Ambion | Breast | PM | F/43 |
| 35-Am-Breast (B64) | 23036 | Ambion | Breast | PM | F/57 |
| 36-Cl-Prostate (P53) | 1070317 | Clontech | Prostate | PB-Pool of 47 | M&F |
| 37-Am-Prostate (P42) | 061P04A | Ambion | Prostate | PM | M/47 |
| 38-Am-Prostate (P59) | 25955 | Ambion | Prostate | PM | M/62 |
| 39-Am-Testis | 111P0104A | Ambion | Testis | PM | M/25 |
| 40-B-Testis | A411147 | Biochain | Testis | PM | M/74 |
| 41-Cl-Testis | 1110320 | Clontech | Testis | PB-Pool of 45 | M&F |
| 42-CG-Adrenal | CG-184-10 | Ichilov | Adrenal | PM | F/81 |
| 43-B-Adrenal | A610374 | Biochain | Adrenal | PM | F/83 |
| 44-B-Heart | A411077 | Biochain | Heart | PB-Pool of 5 | M&F |
| 45-CG-Heart | CG-255-9 | Ichilov | Heart | PM | M/75 |
| 46-CG-Heart | CG-227-1 | Ichilov | Heart | PM | F/36 |
| 47-Am-Liver | 081P0101A | Ambion | Liver | PM | M/64 |
| 48-CG-Liver | CG-93-3 | Ichilov | Liver | PM | F/19 |
| 49-CG-Liver | CG-124-4 | Ichilov | Liver | PM | F/34 |
| 50-Cl-BM | 1110932 | Clontech | Bone Marrow | PM-Pool of 8 | M&F |
| 51-CGEN-Blood | WBC#5 | CGEN | Blood | | M |
| 52-CGEN-Blood | WBC#4 | CGEN | Blood | | M |
| 53-CGEN-Blood | WBC#3 | CGEN | Blood | | M |
| 54-CG-Spleen | CG-267 | Ichilov | Spleen | PM | F/25 |
| 55-CG-Spleen | 111P0106B | Ambion | Spleen | PM | M/25 |
| 56-CG-Spleen | A409246 | Biochain | Spleen | PM | F/12 |
| 56-CG-Thymus | CG-98-7 | Ichilov | Thymus | PM | F/28 |
| 58-Am-Thymus | 101P0101A | Ambion | Thymus | PM | M/14 |
| 59-B-Thymus | A409278 | Biochain | Thymus | PM | M/28 |
| 60-B-Thyroid | A610287 | Biochain | Thyroid | PM | M/27 |
| 61-B-Thyroid | A610286 | Biochain | Thyroid | PM | M/24 |
| 62-CG-Thyroid | CG-119-2 | Ichilov | Thyroid | PM | F/66 |
| 63-Cl-Salivary Gland | 1070319 | Clontech | Salivary Gland | PM-Pool of 24 | M&F |
| 64-Am-Kidney | 111P0101B | Ambion | Kidney | PM-Pool of 14 | M&F |
| 65-Cl-Kidney | 1110970 | Clontech | Kidney | PM-Pool of 14 | M&F |
| 66-B-Kidney | A411080 | Biochain | Kidney | PM-Pool of 5 | M&F |
| 67-CG-Cerebellum | CG-183-5 | Ichilov | Cerebellum | PM | M/74 |
| 68-CG-Cerebellum | CG-212-5 | Ichilov | Cerebellum | PM | M/54 |
| 69-B-Brain | A411322 | Biochain | Brain | PM | M/28 |
| 70-Cl-Brain | 1120022 | Clontech | Brain | PM-Pool of 2 | M&F |
| 71-B-Brain | A411079 | Biochain | Brain | PM-Pool of 2 | M&F |
| 72-CG-Brain | CG-151-1 | Ichilov | Brain | PM | F/86 |
| 73-Am-Skeletal Muscle | 101P013A | Ambion | Skeletal Muscle | PM | F/28 |
| 74-Cl-Skeletal Muscle | 1061038 | Clontech | Skeletal Muscle | PM-Pool of 2 | M&F |

### Materials and Experimental Procedures

RNA preparation—RNA was obtained from Clontech (Franklin Lakes, N.J. USA 07417, www.clontech.com), BioChain Inst. Inc. (Hayward, Calif. 94545 USA www.biochain.com), ABS (Wilmington, Del. 19801, USA, www.absbioreagents.com), Ambion (Austin, Tex. 78744 USA, www.ambion.com), or GOG for ovary samples—Pediatic Cooperative Human Tissue Network, Gynecologic Oncology Group Tissue Bank, Children Hospital of Columbus (Columbus Ohio 43205 USA). Alternatively, RNA was generated from tissue samples using TRI-Reagent (Molecular Research Center), according to Manufacturer's instructions. Tissue and RNA samples were obtained from patients or from postmortem. Total RNA samples were treated with DNaseI (Ambion).

RT PCR—Purified RNA (1 μg) was mixed with 150 ng Random Hexamer primers (Invitrogen) and 500 μM dNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units RNasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 25 μl) was incubated for 50 min at 42° C. and then inactivated at 70° C. for 15 min. The resulting cDNA was diluted 1:20 in TE buffer (10 mM Tris pH=8, 1 mM EDTA pH=8).

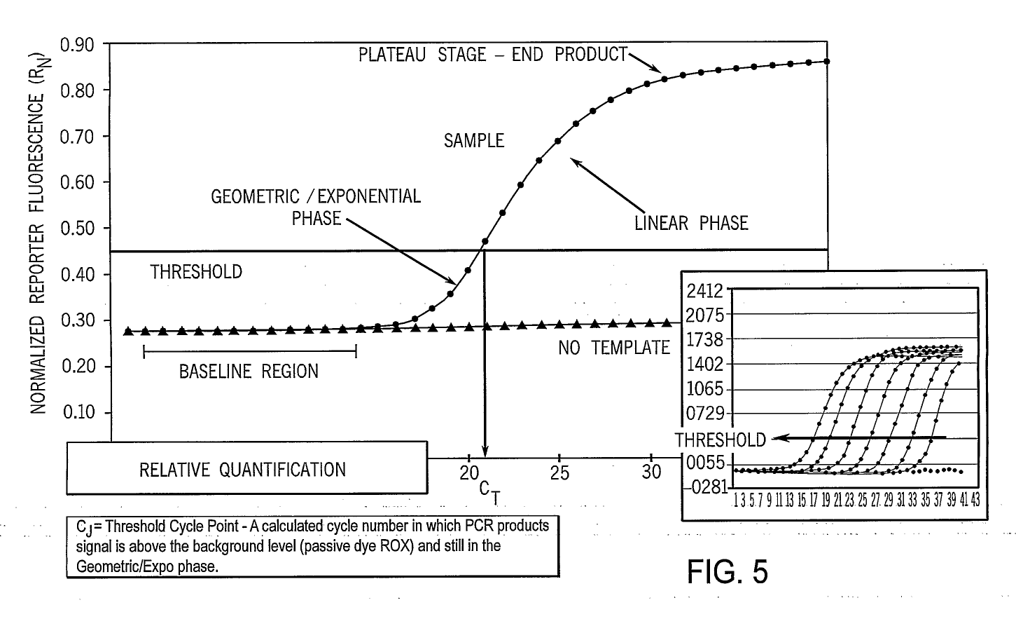

Real-Time RT-PCR analysis—cDNA (5 μL), prepared as described above, was used as a template in Real-Time PCR reactions using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. The relative quantity was calculated using the equation Q=efficiencŷ^{-Ct}. The efficiency of the PCR reaction was calculated from a standard curve, created by using serial dilutions of several reverse transcription (RT) reactions. To minimize inherent differences in the RT reaction, the resulting relative quantities were normalized to the geometric mean of the relative quantities of several housekeeping (HSKP) genes. Schematic summary of quantitative real-time PCR analysis is presented in FIG. 5. As shown, the x-axis shows the cycle number. The C_{T}=Threshold Cycle point, which is the cycle that the amplification curve crosses the fluorescence threshold that was set in the experiment. This point is a calculated cycle number in which PCR product signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

The sequences of the housekeeping genes measured in all the examples below on prostate panel were as follows:

SDHA (GenBank Accession No. NM_004168
(SEQ ID NO:4))
SDHA Forward primer (SEQ ID NO:27):
TGGGAACAAGAGGGCATCTG
SDHA Reverse primer (SEQ ID NO:28):
CCACCACTGCATCAAATTCATG
SDHA-amplicon (SEQ ID NO:29):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323
(SEQ ID NO:6)),
PBGD Forward primer (SEQ ID NO:30):
TGAGAGTGATTCGCGTGGG
PBGD Reverse primer (SEQ ID NO:31):
CCAGGGTACGAGGCTTTCAAT
PBGD-amplicon (SEQ ID NO:32):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194
(SEQ ID NO:5)),
HPRT1 Forward primer (SEQ ID NO:33):
TGACACTGGCAAAACAATGCA
HPRT1 Reverse primer (SEQ ID NO:34):
GGTCCTTTTCACCAGCAAGCT
HPRT1-amplicon (SEQ ID NO:35):
TGACACTGGCAAAACAATGCAGACTTTGCTTTCCTTGGTCAGGCAGTATA
ATCCAAAGATGGTCAAGGTCGCAAGCTTGCTGGTGAAAAGGACC
RPL19 (GenBank Accession No. NM_000981
(SEQ ID NO:7)
RPL19 Forward primer (SEQ ID NO:36):
TGGCAAGAAGAAGGTCTGGTTAG
RPL19 Reverse primer (SEQ ID NO:37):
TGATCAGCCCATCTTTGATGAG
RPL19-amplicon (SEQ ID NO:38):
TGGCAAGAAGAAGGTCTGGTTAGACCCCAATGAGACCAATGAAATCGCCA
ATGCCAACTCCCGTCAGCAGATCCGGAAGCTCATCAAAGATGGGCTGATC
A

The sequences of the housekeeping genes measured in all the examples on ovarian cancer panel were as follows:

SDHA (GenBank Accession No. NM_004168
(SEQ ID NO:4))
SDHA Forward primer (SEQ ID NO:27):
TGGGAACAAGAGGGCATCTG
SDHA Reverse primer (SEQ ID NO:28):
CCACCACTGCATCAAATTCATG
SDHA-amplicon (SEQ ID NO:29):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323
(SEQ ID NO:6)),
PBGD Forward primer (SEQ ID NO:30):
TGAGAGTGATTCGCGTGGG
PBGD Reverse primer (SEQ ID NO:31):
CCAGGGTACGAGGCTTTCAAT
PBGD-amplicon (SEQ ID NO:32):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194
(SEQ ID NO:5)),
HPRT1 Forward primer (SEQ ID NO:33):
TGACACTGGCAAAACAATGCA
HPRT1 Reverse primer (SEQ ID NO:34):
GGTCCTTTTCACCAGCAAGCT
HPRT1-amplicon (SEQ ID NO:35):
TGACACTGGCAAAACAATGCAGACTTTGCTTTCCTTGGTCAGGCAGTATA
ATCCAAAGATGGTCAAGGTCGCAAGCTTGCTGGTGAAAAGGACC
GAPDH (GenBank Accession No. BC026907
(SEQ ID NO:3))
GAPDH Forward primer (SEQ ID NO:39):
TGCACCACCAACTGCTTAGC
GAPDH Reverse primer (SEQ ID NO:40):
CCATCACGCCACAGTTTCC
GAPDH-amplicon (SEQ ID NO:41):
TGCACCACCAACTGCTTAGCACCCCTGGCCAAGGTCATCCATGACAACTT
TGGTATCGTGGAAGGACTCATGACCACAGTCCATGCCATCACTGCCACCC
AGAAGACTGTGGATGG

The sequences of the housekeeping genes measured in all the examples on colon cancer tissue testing panel were as follows:

PBGD (GenBank Accession No. BC019323
(SEQ ID NO:6)),
PBGD Forward primer (SEQ ID NO:30):
TGAGAGTGATTCGCGTGGG
PBGD Reverse primer (SEQ ID NO:31):
CCAGGGTACGAGGCTTTCAAT
PBGD-amplicon (SEQ ID NO:32):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194
(SEQ ID NO:5)),
HPRT1 Forward primer (SEQ ID NO:33):
TGACACTGGCAAAACAATGCA
HPRT1 Reverse primer (SEQ ID NO:34):
GGTCCTTTTCACCAGCAAGCT
HPRT1-amplicon (SEQ ID NO:35):
TGACACTGGCAAAACAATGCAGACTTTGCTTTCCTTGGTCAGGCAGTATA
ATCCAAAGATGGTCAAGGTCGCAAGCTTGCTGGTGAAAAGGACC
G6PD (GenBank Accession No. NM_000402
(SEQ ID NO:8)
G6PD Forward primer (SEQ ID NO:42):
gaggccgtcaccaagaacat
G6PD Reverse primer (SEQ ID NO:43):
ggacagccggtcagagctc
G6PD-amplicon (SEQ ID NO:44):
gaggccgtcaccaagaacattcacgagtcctgcatgagccagataggctg
gaaccgcatcatcgtggagaagcccttcgggagggacctgcagagctctg
accggctgtcc
RPS27A (GenBank Accession No. NM_002954
(SEQ ID NO:1))
RPS27A Forward primer (SEQ ID NO:45):
CTGGCAAGCAGCTGGAAGAT
RPS27A Reverse primer (SEQ ID NO:46):
TTTCTTAGCACCACCACGAAGTC
RPS27A-amplicon (SEQ ID NO:47):
CTGGCAAGCAGCTGGAAGATGGACGTACTTTGTCTGACTACAATATTCAA
AAGGAGTCTACTCTTCATCTTGTGTTGAGACTTCGTGGTGGTGCTAAGAA
A

The sequences of the housekeeping genes measured in all the examples in testing panel were as follows:

Ubiquitin (GenBank Accession No. BC000449
(SEQ ID NO:9))
Ubiquitin Forward primer (SEQ ID NO:48):
ATTTGGGTCGCGGTTCTTG
Ubiquitin Reverse primer (SEQ ID NO:49):
TGCCTTGACATTCTCGATGGT
Ubiquitin-amplicon (SEQ ID NO:50):
ATTTGGGTCGCGGTTCTTGTTTGTGGATCGCTGTGATCGTCACTTGACAA
TGCAGATCTTCGTGAAGACTCTGACTGGTAAGACCATCACCCTCGAGGTT
GAGCCCAGTGACACCATCGAGAATGTCAAGGCA
SDHA (GenBank Accession No. NM_004168
(SEQ ID NO:4))
SDHA Forward primer (SEQ ID NO:27):
TGGGAACAAGAGGGCATCTG
SDHA Reverse primer (SEQ ID NO:28):
CCACCACTGCATCAAATTCATG
SDHA-amplicon (SEQ ID NO:29):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323
(SEQ ID NO:6)),
PBGD Forward primer (SEQ ID NO:30):
TGAGAGTGATTCGCGTGGG
PBGD Reverse primer (SEQ ID NO:31):
CCAGGGTACGAGGCTTTCAAT
PBGD-amplicon (SEQ ID NO:32):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194
(SEQ ID NO:5)),
HPRT1 Forward primer (SEQ ID NO:33):
TGACACTGGCAAAACAATGCA
HPRT1 Reverse primer (SEQ ID NO:34):
GGTCCTTTTCACCAGCAAGCT
HPRT1-amplicon (SEQ ID NO:35):
TGACACTGGCAAAACAATGCAGACTTTGCTTTCCTTGGTCAGGCAGTATA
ATCCAAAGATGGTCAAGGTCGCAAGCTTGCTGGTGAAAAGGACC

The sequences of the housekeeping genes measured in all the examples on breast cancer panel were as follows:

G6PD (GenBank Accession No. NM_000402
(SEQ ID NO:8))
G6PD Forward primer (SEQ ID NO:42):
gaggccgtcaccaagaacat
G6PD Reverse primer (SEQ ID NO:43):
ggacagccggtcagagctc
G6PD-amplicon (SEQ ID NO:44):
gaggccgtcaccaagaacattcacgagtcctgcatgagccagataggctg
gaaccgcatcatcgtggagaagcccttcgggagggacctgcagagctctg
accggctgtcc
SDHA (GenBank Accession No. NM_004168
(SEQ ID NO:4))
SDHA Forward primer (SEQ ID NO:27):
TGGGAACAAGAGGGCATCTG
SDHA Reverse primer (SEQ ID NO:28):
CCACCACTGCATCAAATTCATG
SDHA-amplicon (SEQ ID NO:29):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323
(SEQ ID NO:6)),
PBGD Forward primer (SEQ ID NO:30):
TGAGAGTGATTCGCGTGGG
PBGD Reverse primer (SEQ ID NO:31):
CCAGGGTACGAGGCTTTCAAT
PBGD-amplicon (SEQ ID NO:32):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194
(SEQ ID NO:5)),
HPRT1 Forward primer (SEQ ID NO:33):
TGACACTGGCAAAACAATGCA
HPRT1 Reverse primer (SEQ ID NO:34):
GGTCCTTTTCACCAGCAAGCT
HPRT1-amplicon (SEQ ID NO:35):
TGACACTGGCAAAACAATGCAGACTTTGCTTTCCTTGGTCAGGCAGTATA
ATCCAAAGATGGTCAAGGTCGCAAGCTTGCTGGTGAAAAGGACC

The sequences of the housekeeping genes measured in all the examples on normal tissue samples panel were as follows:

RPL19 (GenBank Accession No. NM_000981
(SEQ ID NO:7)
RPL19 Forward primer (SEQ ID NO:36):
TGGCAAGAAGAAGGTCTGGTTAG
RPL19 Reverse primer (SEQ ID NO:37):
TGATCAGCCCATCTTTGATGAG
RPL19-amplicon (SEQ ID NO:38):
TGGCAAGAAGAAGGTCTGGTTAGACCCCAATGAGACCAATGAAATCGCCA
ATGCCAACTCCCGTCAGCAGATCCGGAAGCTCATCAAAGATGGGCTGATC
A
TATA box (GenBank Accession No. NM_003194
(SEQ ID NO:2)),
TATA box Forward primer (SEQ ID NO:51):
CGGTTTGCTGCGGTAATCAT
TATA box Reverse primer (SEQ ID NO:52):
TTTCTTGCTGCCAGTCTGGAC
TATA box-amplicon (SEQ ID NO:53):
CGGTTTGCTGCGGTAATCATGAGGATAAGAGAGCCACGAACCACGGCACT
GATTTTCAGTTCTGGGAAAATGGTGTGCACAGGAGCCAAGAGTGAAGAAC
AGTCCAGACTGGCAGCAAGAAA
Ubiquitin (GenBank Accession No. BC000449
(SEQ ID NO:9))
Ubiquitin Forward primer (SEQ ID NO:48):
ATTTGGGTCGCGGTTCTTG
Ubiquitin Reverse primer (SEQ ID NO:49):
TGCCTTGACATTCTCGATGGT
Ubiquitin-amplicon (SEQ ID NO:50):
ATTTGGGTCGCGGTTCTTGTTTGTGGATCGCTGTGATCGTCACTTGACAA
TGCAGATCTTCGTGAAGACTCTGACTGGTAAGACCATCACCCTCGAGGTT
GAGCCCAGTGACACCATCGAGAATGTCAAGGCA
SDHA (GenBank Accession No. NM_004168
(SEQ ID NO:4))
SDHA Forward primer (SEQ ID NO:27):
TGGGAACAAGAGGGCATCTG
SDHA Reverse primer (SEQ ID NO:28):
CCACCACTGCATCAAATTCATG
SDHA-amplicon (SEQ ID NO:29):
TGGGAACAAGAGGGCATCTGCTAAAGTTTCAGATTCCATTTCTGCTCAGT
ATCCAGTAGTGGATCATGAATTTGATGCAGTGGTGG

### Actual Marker Examples

The following examples relate to specific actual marker examples. It should be noted that Table and Figures numbering is restarted within each example related to a particular Cluster, as indicated by the titles below.

### Description for Cluster N56180

Cluster N56180 features 7 transcript(s) and 22 segment(s) of interest, the names for which are given in Tables 8 and 9, respectively. The selected protein variants are given in table 10.

TABLE 8
Transcripts of interest
Transcript Name

N56180_T1 (SEQ ID NO: 54)
N56180_T3 (SEQ ID NO: 55)
N56180_T4 (SEQ ID NO: 56)
N56180_T5 (SEQ ID NO: 57)
N56180_T6 (SEQ ID NO: 58)
N56180_T7 (SEQ ID NO: 59)
N56180_T8 (SEQ ID NO: 60)

TABLE 9
Segments of interest

| Segment Name | Corresponding Transcripts |
|---|---|
| N56180_node_2 (SEQ ID NO: 61) | N56180_T1 (SEQ ID NO: 54), N56180_T3 |
| | (SEQ ID NO: 55), N56180_T4 (SEQ ID NO: 56) |
| N56180_node_4 (SEQ ID NO: 62) | N56180_T1 (SEQ ID NO: 54), N56180_T3 |
| | (SEQ ID NO: 55), N56180_T4 (SEQ ID |
| | NO: 56), N56180_T5 (SEQ ID NO: 57) and |
| | N56180_T8 (SEQ ID NO: 60) |
| N56180_node_6 (SEQ ID NO: 63) | N56180_T3 (SEQ ID NO: 55) |
| N56180_node_20 (SEQ ID NO: 64) | N56180_T1 (SEQ ID NO: 54), N56180_T3 |
| | (SEQ ID NO: 55), N56180_T4 (SEQ ID |
| | NO: 56), N56180_T5 (SEQ ID NO: 57), |
| | N56180_T6 (SEQ ID NO: 58) and |
| | N56180_T8 (SEQ ID NO: 60) |
| N56180_node_22 (SEQ ID NO: 65) | N56180_T8 (SEQ ID NO: 60) |
| N56180_node_28 (SEQ ID NO: 66) | N56180_T7 (SEQ ID NO: 59) |
| N56180_node_34 (SEQ ID NO: 67) | N56180_T1 (SEQ ID NO: 54), N56180_T3 |
| | (SEQ ID NO: 55), N56180_T4 (SEQ ID |
| | NO: 56), N56180_T5 (SEQ ID NO: 57), |
| | N56180_T6 (SEQ ID NO: 58) and |
| | N56180_T7 (SEQ ID NO: 59) |
| N56180_node_36 (SEQ ID NO: 68) | N56180_T1 (SEQ ID NO: 54), N56180_T3 |
| | (SEQ ID NO: 55), N56180_T4 (SEQ ID |
| | NO: 56), N56180_T5 (SEQ ID NO: 57), |
| | N56180_T6 (SEQ ID NO: 58) and |
| | N56180_T7 (SEQ ID NO: 59) |
| N56180_node_0 (SEQ ID NO: 69) | N56180_T5 (SEQ ID NO: 57) |
| N56180_node_3 (SEQ ID NO: 70) | N56180_T1 (SEQ ID NO: 54), N56180_T3 |
| | (SEQ ID NO: 55), N56180_T4 (SEQ ID |
| | NO: 56) and N56180_T8 (SEQ ID NO: 60) |
| N56180_node_8 (SEQ ID NO: 71) | N56180_T1 (SEQ ID NO: 54), N56180_T3 |
| | (SEQ ID NO: 55), N56180_T4 (SEQ ID |
| | NO: 56), N56180_T5 (SEQ ID NO: 57) and |
| | N56180_T8 (SEQ ID NO: 60). Table 29 |
| N56180_node_10 (SEQ ID NO: 72) | N56180_T1 (SEQ ID NO: 54), N56180_T3 |
| | (SEQ ID NO: 55), N56180_T4 (SEQ ID |
| | NO: 56) and N56180_T8 (SEQ ID NO: 60) |
| N56180_node_12 (SEQ ID NO: 73) | N56180_T1 (SEQ ID NO: 54), N56180_T3 |
| | (SEQ ID NO: 55) and N56180_T8 (SEQ ID NO: 60) |
| N56180_node_14 (SEQ ID NO: 74) | N56180_T1 (SEQ ID NO: 54), N56180_T3 |
| | (SEQ ID NO: 55), N56180_T5 (SEQ ID |
| | NO: 57) and N56180_T8 (SEQ ID NO: 60) |
| N56180_node_16 (SEQ ID NO: 75) | N56180_T1 (SEQ ID NO: 54) |
| N56180_node_18 (SEQ ID NO: 76) | N56180_T6 (SEQ ID NO: 58) |
| N56180_node_24 (SEQ ID NO: 77) | N56180_T1 (SEQ ID NO: 54), N56180_T3 |
| | (SEQ ID NO: 55), N56180_T4 (SEQ ID |
| | NO: 56), N56180_T5 (SEQ ID NO: 57) and |
| | N56180_T6 (SEQ ID NO: 58) |
| N56180_node_26 (SEQ ID NO: 78) | N56180_T1 (SEQ ID NO: 54), N56180_T3 |
| | (SEQ ID NO: 55), N56180_T4 (SEQ ID |
| | NO: 56), N56180_T5 (SEQ ID NO: 57) and |
| | N56180_T6 (SEQ ID NO: 58) |
| N56180_node_29 (SEQ ID NO: 79) | N56180_T1 (SEQ ID NO: 54), N56180_T3 |
| | (SEQ ID NO: 55), N56180_T4 (SEQ ID |
| | NO: 56), N56180_T5 (SEQ ID NO: 57), |
| | N56180_T6 (SEQ ID NO: 58) and |
| | N56180_T7 (SEQ ID NO: 59) |
| N56180_node_31 (SEQ ID NO: 80) | N56180_T1 (SEQ ID NO: 54), N56180_T3 |
| | (SEQ ID NO: 55), N56180_T4 (SEQ ID |
| | NO: 56), N56180_T5 (SEQ ID NO: 57), |
| | N56180_T6 (SEQ ID NO: 58) and |
| | N56180_T7 (SEQ ID NO: 59) |
| N56180_node_33 (SEQ ID NO: 81) | N56180_T1 (SEQ ID NO: 54), N56180_T3 |
| | (SEQ ID NO: 55), N56180_T4 (SEQ ID |
| | NO: 56), N56180_T5 (SEQ ID NO: 57), |
| | N56180_T6 (SEQ ID NO: 58) and |
| | N56180_T7 (SEQ ID NO: 59) |
| N56180_node_35 (SEQ ID NO: 82) | N56180_T1 (SEQ ID NO: 54), N56180_T3 |
| | (SEQ ID NO: 55), N56180_T4 (SEQ ID |
| | NO: 56), N56180_T5 (SEQ ID NO: 57), |
| | N56180_T6 (SEQ ID NO: 58) and |
| | N56180_T7 (SEQ ID NO: 59) |

TABLE 10
Proteins of interest

| Protein Name | Corresponding Transcript(s) |
|---|---|
| N56180_P2 (SEQ ID NO: 84) | N56180_T1 (SEQ ID NO: 54) |
| N56180_P4 (SEQ ID NO: 85) | N56180_T3 (SEQ ID NO: 55) |
| N56180_P5 (SEQ ID NO: 86) | N56180_T4 (SEQ ID NO: 56) |
| N56180_P6 (SEQ ID NO: 87) | N56180_T5 (SEQ ID NO: 57) |
| N56180_P7 (SEQ ID NO: 88) | N56180_T6 (SEQ ID NO: 58) |
| N56180_P8 (SEQ ID NO: 89) | N56180_T7 (SEQ ID NO: 59) |
| N56180_P9 (SEQ ID NO: 90) | N56180_T8 (SEQ ID NO: 60) |

These sequences are variants of the known protein Calsequestrin, cardiac muscle isoform precursor (SwissProt accession identifier CAQ2_HUMAN (SEQ ID NO:83); known also according to the synonyms Calsequestrin 2), referred to herein as the previously known protein.

Protein Calsequestrin, cardiac muscle isoform precursor (SEQ ID NO: 83) is known or believed to have the following function(s): Calsequestrin is a high-capacity, moderate affinity, calcium-binding protein and thus acts as an internal calcium store in muscle. The release of calcium bound to calsequestrin through a calcium release channel triggers muscle contraction. Binds 40 to 50 moles of calcium (By similarity). Known polymorphisms for this sequence are as shown in Table 11.

TABLE 11
Amino acid mutations for Known Protein

| SNP position(s) on amino | |
|---|---|
| acid sequence | Comment |
| 307 | D -> H (in VTSIP). /FTId = VAR_016075. |
| 67 | Q -> P |

Protein Calsequestrin, cardiac muscle isoform precursor (SEQ ID NO: 83) localization is believed to be This isoform of calsequestrin occurs in the sarcoplasmic reticulum's terminal cisternae luminal spaces of cardiac and slow skeletal muscle cells.

The following GO Annotation(s) apply to the previously known protein. The following annotation(s) were found: striated muscle contraction; heart development; muscle development, which are annotation(s) related to Biological Process; calcium storage, which are annotation(s) related to Molecular Function; and smooth endoplasmic reticulum, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of N56180) may optionally have one or more of the following utilities, as described with regard to the Table 12 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 12
Utilities for Variants of N56180, related to Calsequestrin, cardiac muscle isoform:
CAQ2_HUMAN (SEQ ID NO: 83)

| | | |
|---|---|---|
| Biomarker for | calsequestrin 2 mutations causes | Circ Res. 2002 Oct |
| catecholaminergic polymorphic | severe forms of | 18; 91(8): e21-6. |
| ventricular tachycardia | catecholaminergic polymorphic | Am J Hum Genet. 2001 |
| (CPVT) | ventricular tachycardia | Dec; 69(6): 1378-84. Epub 2001 |
| | | Oct 25. |
| | | Cold Spring Harb Symp Quant |
| | | Biol. 2002; 67: 333-7. |
| | | Trends Cardiovasc Med. 2003 |
| | | May; 13(4): 148-51. |
| Marker for predisposition for | Abnormal calcium signaling and | Circ Res. 2004 Mar |
| sudden cardiac death | sudden cardiac death associated | 5; 94(4): 471-7. Epub 2004 Jan |
| | with mutation of calsequestrin | 08. |
| cardiotoxicity | Interaction between cardiac | Mol Pharmacol. 2005 |
| | calsequestrin and drugs with | Jan; 67(1): 97-104. Epub 2004 |
| | known cardiotoxicity. | Oct 18. |
| Biomarker for heart failure | transgenic mice overexpressing | J Pharmacol Exp Ther. 2000 |
| | both NCX and CSQ | Aug; 294(2): 648-57. |
| | (calsequestrin) | |
| dystrophic skeletal muscle. | Subproteomics analysis of Ca+- | Eur J Biochem. 2004 |
| | binding proteins demonstrates | Oct; 271(19): 3943-52. |
| | decreased calsequestrin | |
| | expression in dystrophic mouse | |
| | skeletal muscle. | |
| left ventricular hypertrophy, | Cardiac-specific overexpression | J Mol Cell Cardiol. 2000 |
| depressed force-frequency | of calsequestrin results in left | Sep; 32(9): 1735-44. |
| relation and pulsus alternans | ventricular hypertrophy, | |
| | depressed force-frequency | |
| | relation and pulsus alternans in | |
| | vivo. (in mice) | |
| Abnormal Ca2+ release and | CASQ2), as well as other genes | Hum Mol Genet. 2005 Mar 9; |
| catecholamine-induced | encoding proteins involved in | [Epub ahead of print] |
| arrhythmias in mitochondrial | SR Ca(2+) handling, showed | |
| cardiomyopathy | decreased expression in Tfam | |
| | knockout hearts | |
| ventricular arrhythmias | Abnormal restitution of the | Biol Res. 2004; 37(4): 603-7. |
| | Ca2+ release channels from a | |
| | luminal Ca-dependent refractory | |
| | state could account for | |
| | ventricular arrhythmias | |
| | associated with mutations in the | |
| | CASQ2 gene. | |
| impairs cardiac function | Overexpression of the | Biochem J. 2003 Apr 1; 371(Pt |
| (Prenatal glucocorticoid | conserved Ca(2+)-binding | 1): 61-9. |
| overexposure) | proteins calreticulin and | |
| | calsequestrin impairs cardiac | |
| | function, leading to premature | |
| | death. | |
| | Prenatal glucocorticoid | |
| | overexposure at the higher dose | |
| | decreased calreticulin protein | |
| | expression (26%; P < 0.05) but | |
| | increased calsequestrin protein | |
| | expression, both 55 and 63 kDa | |
| | bands, by 87% (P < 0.01) and | |
| | 78% (P < 0.01); only the 55 kDa | |
| | calsequestrin band was | |
| | increased at the lower dose | |
| | (66%; P < 0.05). | |
| Cardiac hypertrophy | overexpression of calsequestrin | Proc Natl Acad Sci USA. 2004 |
| | | Mar 2; 101(9): 3106-11. Epub |
| | | 2004 Feb 20. |
| hypertrophy, heart failure, and | Transgenic cardiac | Am J Physiol Heart Circ |
| premature death. | overexpression of canine | Physiol. 2004 |
| | calsequestrin (CSQ) showed | Sep; 287(3): H1096-103. Epub |
| | hypertrophy, heart failure, and | 2004 May 6 |
| | premature death. | |
| cardiac hypertrophy and dilated | Transgenic mice overexpressing | Cell Calcium. 2002 |
| cardiomyopathy | CSQ at the age of 7 weeks | Jul; 32(1): 21-9. |
| | exhibit concentric cardiac | |
| | hypertrophy, and by 13 weeks | |
| | the condition progresses to | |
| | dilated cardiomyopathy. | |
| depressed cardiovascular | Cardiac-specific overexpression | J Biol Chem. 1998 Oct |
| function and hypertrophy | of mouse cardiac calsequestrin | 23; 273(43): 28470-7. |
| | is associated with depressed | |
| | cardiovascular function and | |
| | hypertrophy in transgenic mice. | |

According to other optional embodiments of the present invention, variants of this cluster according to the present invention (amino acid and/or nucleic acid sequences of N56180) may optionally have one or more of the following utilities, some of which are related to utilities described above. It should be noted that these utilities are optionally and preferably suitable for human and non-human animals as subjects, except where otherwise noted.

A non-limiting example of such a utility is the detection, diagnosis and/or determination of ovarian or uterine serous papillary carcinoma. The method comprises detecting a N56180 variant, for example a variant protein, protein fragment, peptide, polynucleotide, polynucleotide fragment and/or oligonucleotide as described herein, optionally and preferably in a serum sample. The expression levels of the N56180 variant as determined in a patient can be further compared to those in a normal individual.

At least 5-fold higher expression of the known CAQ2_HUMAN (SEQ ID NO:83) gene in uterine serous papillary carcinoma as compared with Normal Endometrial Epithelial Cells is described with regard to PCT Application No. WO04108896, hereby incorporated by reference as if fully set forth herein.

Oligonucleotide microarrays were used to profile and compare gene expression patterns between uterine serous papillary carcinoma and ovarian serous papillary carcinoma or normal endometrial epithelial cells. mRNA fingerprints readily distinguish the more biologically aggressive and chemotherapy resistant USPC from OSPC or NEC. The known CAQ2_HUMAN (SEQ ID NO:83) gene is strikingly overexpressed in uterine serous papillary carcinoma as compared with Normal Endometrial Epithelial Cells and may therefore represent a novel diagnostic and therapeutic marker for this highly aggressive subset of endometrial tumors.

Another non-limiting example of such a utility is the detection, diagnosis and/or determination the condition of an ailing organ. Although applicable to numerous organ and organ systems, the CAQ2 variants can be preferably used as marker for diagnosing and distinguishing congestive heart failure. The method comprises detecting a N56180 variant, for example a variant protein, protein fragment, peptide, polynucleotide, polynucleotide fragment and/or oligonucleotide as described herein, optionally and preferably in a serum sample. The expression levels of the N56180 variant as determined in a patient can be further compared to those in a normal individual, and can be used for monitoring disease progression and efficacy of therapeutic agents.

Use of the known CAQ2_HUMAN (SEQ ID NO:83) gene within a diagnostic kit for rapidly diagnosing organ damage, and more preferably heart damage, in a patient is described with regard to WO03020123 patent application, hereby incorporated by reference as if fully set forth herein. The WO03020123 patent application also describes the use of the known CAQ2_HUMAN (SEQ ID NO:83) gene for predicting cardiac mortality rate in a patient.

According to preferred embodiments of the present invention, the levels of the N56180 variant can be used for detection, diagnosis and/or determination the condition of an ailing organ, more preferably for detection, diagnosis and/or determination of heart damage and for predicting cardiac mortality rate in a patient.

The gene STB2 (NM_{—}138959 (SEQ ID NO:696); NP_{—}620409 (SEQ ID NO:697)) (VANGL1) is antisense with CASQ2 gene on human chromosome 1p13 in a tail to tail orientation, and may therefore be co-regulated and co-expressed with one or more N56180 variants according to the present invention, and hence may have one or more diagnostic utilities of N56180 variants according to the present invention as described herein.

STB1 and STB2 genes are located around cancer susceptibility loci or recombination hot spots in the human genome. STB1 is moderately expressed in K-562 (leukemia), G-361 (melanoma), and MKN7 (gastric cancer) cells. STB2 is highly expressed in MKN28, MKN74 (gastric cancer), BxPC-3, PSN-1, and Hs766T (pancreatic cancer) cells. On the other hand, STB1 and STB2 are significantly down-regulated in several cancer cell lines and primary tumors. Xenopus homologue of human STB1 and STB2 regulates negatively the WNT-beta-catenin signaling pathway. Loss-of-function mutations of genes encoding negative regulators of WNT-beta-catenin signaling pathway lead to carcinogenesis. Based on functional aspects and human chromosomal loci, the STB1 and STB2 genes are predicted to be potent tumor suppressor gene candidates. STB1 and STB2 might be suitable targets for tissue engineering in the field of re-generative medicine and for chemoprevention and treatment in the field of clinical oncology. (Katoh M. Int J Mol Med. 2002 July; 10(1): 11-5).

Table 13 below describes diagnostic utilities for the cluster N56180 that were found through microarrays, including the statistical significance thereof and a reference. One or more N56180 variants according to the present invention may optionally have one or more of these utilities.

TABLE 13

| | Statistical | |
|---|---|---|
| Diagnostic utility | significance | reference |
| Gene over expressed in | 1.2E−4 | LaTulippe E, Gerald WL Cancer Research (2002) |
| Primary Prostate Carcinoma | | Comprehensive Gene Expression Analysis of |
| (vs. metastasis). | | Prostate Cancer Reveals Distinct Transcriptional |
| | | Programs Associated with Metastatic Disease |
| Gene over expressed in Non- | 1.1E−10 | Nutt CL, Louis DN. Cancer Res (2003) Gene |
| Classic Glioma (vs. | | expression-based classification of malignant gliomas |
| Classical). | | correlates better with survival than histological |
| | | classification. |
| Gene over expressed in | | GNF database |
| leiomyomas (vs. normal | | (http://www.ncbi.nlm.nih.gov/projects/geo/): |
| uterus). | | GDS484, probe ID: 207317_s_at. |
| Gene under expressed in | | GNF database |
| ischemic cardiomyopathy | | (http://www.ncbi.nlm.nih.gov/projects/geo/): |
| (vs. idiopathic dilated | | GDS651, probe ID: 207317_s_at. |
| cardiomyopathy). Can be | | |
| used in combination with | | |
| other CHF and MI markers | | |
| for differential diagnosis and | | |
| treatment regiment decisions. | | |

Other non-limiting exemplary utilities for N56180 variants according to the present invention are described in greater detail below and also with regard to the previous section on clinical utility.

The heart-selective diagnostic marker prediction engine provided the following results with regard to cluster N56180. Predictions were made for selective expression of transcripts of this contig in heart tissue, according to the previously described methods. The numbers on the y-axis of the first figure below refer to weighted expression of ESTs in each category, as “parts per million” (ratio of the expression of ESTs for a particular cluster to the expression of all ESTs in that category, according to parts per million).

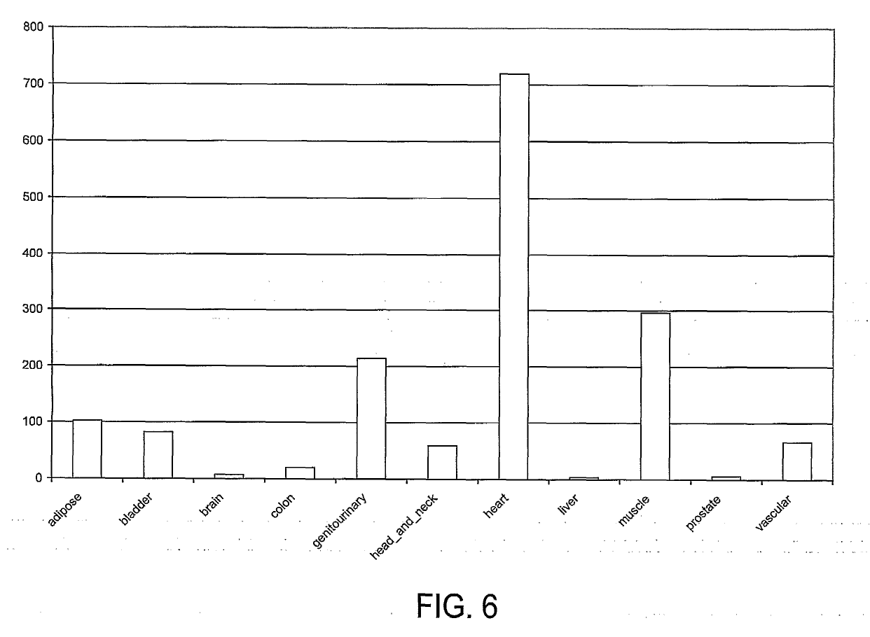

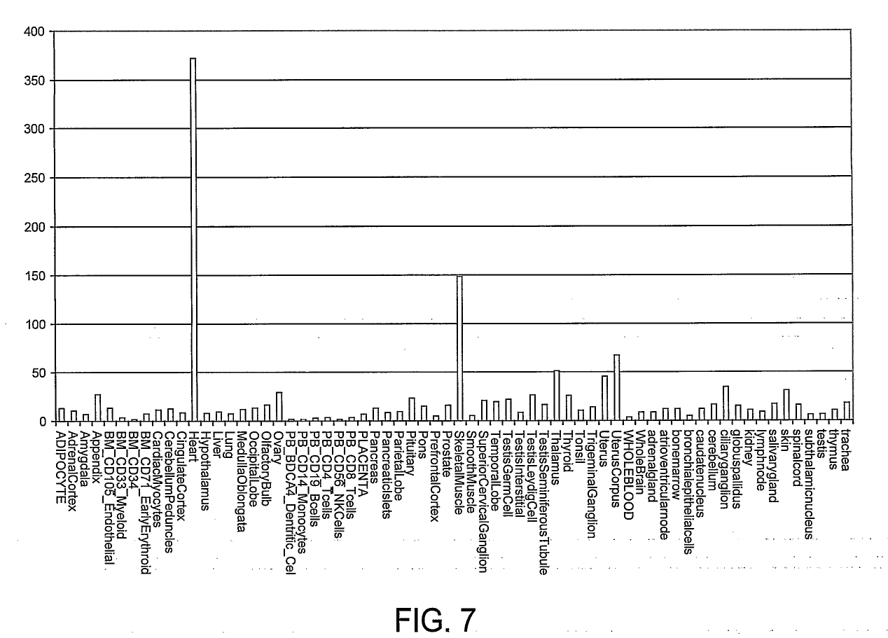

Overall, the following results were obtained as shown with regard to the histogram in FIG. 6, concerning the number of heart-specific clones in libraries/sequences; as well as with regard to the histogram in FIG. 7, concerning the actual expression of oligonucleotides in various tissues, including heart.

This cluster was found to be selectively expressed in heart for the following reasons: in a comparison of the ratio of expression of the cluster in heart specific ESTs to the overall expression of the cluster in non-heart ESTs, which was found to be 11.2; the ratio of expression of the cluster in heart specific ESTs to the overall expression of the cluster in muscle-specific ESTs which was found to be 2.4; and fisher exact test P-values were computed both for library and weighted clone counts to check that the counts are statistically significant, and were found to be 4.30E-14.

One particularly important measure of specificity of expression of a cluster in heart tissue is the previously described comparison of the ratio of expression of the cluster in heart as opposed to muscle. This cluster was found to be specifically expressed in heart as opposed to non-heart ESTs as described above. However, many proteins have been shown to be generally expressed at a higher level in both heart and muscle, which is less desirable. For this cluster, as described above, the ratio of expression of the cluster in heart specific ESTs to the overall expression of the cluster in muscle-specific ESTs which was found to be 11.2, which clearly supports specific expression in heart tissue.

As noted above, cluster N56180 features 7 transcript(s), which were listed in Table 8 above. These transcript(s) encode for protein(s) which are variant(s) of protein Calsequestrin, cardiac muscle isoform precursor (SEQ ID NO: 83). A description of each variant protein according to the present invention is now provided.

Variant protein N56180_P2 (SEQ ID NO:84) according to the present invention has an amino acid sequence; it is encoded by transcript(s) N56180_T1 (SEQ ID NO:54). An alignment is given to the known protein (Calsequestrin, cardiac muscle isoform precursor (SEQ ID NO: 83)). One or more alignments to one or more previously published protein sequences are given in the alignment table located on the attached CDROM. A brief description of the relationship of the variant protein according to the present invention to each such aligned protein is as follows:

Comparison report between N56180_P2 (SEQ ID NO:84) and CAQ2_HUMAN (SEQ ID NO:83):

1. An isolated chimeric polypeptide encoding for N56180_P2 (SEQ ID NO:84), comprising a first amino acid sequence being at least 90% homologous to MKRTHLFIVGIYFLSSCRAEEGLNFPTYDGKDRVVSLSEKNFKQVLKKYDLLCLYYHEPVSSDKVT QKQFQLKEIVLELVAQVLEHKAIGFVMVDAKKEAKLAKKLGFDEEGSLYILKGDRTIEFDGEFAA DVLVEFLLDLIEDPVEIISSKLEVQAFERIEDYIKLIGFFKSEDSEYYKAFEEAAEHFQPYIKFFATFD KGV corresponding to amino acids 1-203 of CAQ2_HUMAN (SEQ ID NO:83), which also corresponds to amino acids 1-203 of N56180_P2 (SEQ ID NO:84), and a second amino acid sequence being at least 70%, optionally at least 80%, preferably at least 85%, more preferably at least 90% and most preferably at least 95% homologous to a polypeptide having the sequence LWLTPVIPTLWEADGGGLHEPWSWRPAWATWLQRNYL (SEQ ID NO: 628) corresponding to amino acids 204-240 of N56180_P2 (SEQ ID NO:84), wherein said first amino acid sequence and second amino acid sequence are contiguous and in a sequential order.

2. An isolated polypeptide encoding for a tail of N56180_P2 (SEQ ID NO:84), comprising a polypeptide being at least 70%, optionally at least about 80%, preferably at least about 85%, more preferably at least about 90% and most preferably at least about 95% homologous to the sequence LWLTPVIPTLWEADGGGLHEPWSWRPAWATWLQRNYL (SEQ ID NO: 628) in N56180_P2 (SEQ ID NO:84).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The glycosylation sites of variant protein N56180_P2 (SEQ ID NO:84), as compared to the known protein Calsequestrin, cardiac muscle isoform precursor (SEQ ID NO: 83), are described in Table 14 (given according to their position(s) on the amino acid sequence in the first column; the second column indicates whether the glycosylation site is present in the variant protein; and the last column indicates whether the position is different on the variant protein).

TABLE 14
Glycosylation site(s)

| Position(s) on known | Present in | Position in |
|---|---|---|
| amino acid sequence | variant protein? | variant protein? |
| 335 | No | |

The variant protein has the following domains, as determined by using InterPro. The domains are described in Table 15:

TABLE 15
InterPro domain(s)

| | Domain | | |
|---|---|---|---|
| InterPro ID | description | Analysis type | Position(s) on protein |
| IPR001393 | Calsequestrin | FPrintScan | 113-142, 154-183, |
| | | | 184-213, 20-43, 47-76, |
| | | | 77-106 |
| IPR001393 | Calsequestrin | HMMPfam | 2-203 |
| IPR001393 | Calsequestrin | ScanRegExp | 20-34 |

Variant protein N56180 P2 (SEQ ID NO:84) is encoded by the following transcript(s): N56180_T1 (SEQ ID NO:54). The coding portion of transcript N56180_T1 (SEQ ID NO:54) starts at position 242 and ends at position 961.

Variant protein N56180_P4 (SEQ ID NO:85) according to the present invention has an amino acid sequence; it is encoded by transcript(s) N56180_T3 (SEQ ID NO:55). An alignment is given to the known protein (Calsequestrin, cardiac muscle isoform precursor (SEQ ID NO: 83)). One or more alignments to one or more previously published protein sequences are given in the alignment table located on the attached CDROM. A brief description of the relationship of the variant protein according to the present invention to each such aligned protein is as follows:

Comparison report between N56180_P4 (SEQ ID NO:85) and CAQ2_HUMAN (SEQ ID NO:83):

1. An isolated chimeric polypeptide encoding for N56180_P4 (SEQ ID NO:85), comprising a first amino acid sequence being at least 90% homologous to MKRTHLFIVGIYFLSSCRAEEGLNFPTYDGKDRVVSLSEKNFKQVLKKYDLLCLYYHEPVSSDKVT QKQFQLKEIVLE corresponding to amino acids 1-78 of CAQ2_HUMAN (SEQ ID NO:83), which also corresponds to amino acids 1-78 of N56180_P4 (SEQ ID NO:85), a second amino acid sequence being at least 70%, optionally at least 80%, preferably at least 85%, more preferably at least 90% and most preferably at least 95% homologous to a polypeptide having the sequence HWQISQWWLHFQTPREEGKMKLLELSESADGAAWKRWGGNSNTHRIQ (SEQ ID NO: 629) corresponding to amino acids 79-125 of N56180_P4 (SEQ ID NO:85), and a third amino acid sequence being at least 90% homologous to LVAQVLEHKAIGFVMVDAKKEAKLAKKLGFDEEGSLYILKGDRTIEFDGEFAADVLVEFLLDLIED PVEIISSKLEVQAFERIEDYIKLIGFFKSEDSEYYKAFEEAAEHFQPYIKFFATFDKGVAKKLSLKMN EVDFYEPFMDEPIAIPNKPYTEEELVEFVKEHQRPTLRRLRPEEMFETWEDDLNGIHIVAFAEKSDP DGYEFLEILKQVARDNTDNPDLSILWIDPDDFPLLVAYWEKTFKIDLFRPQIGVVNVTDADSVWME IPDDDDLPTAEELEDWIEDVLSGKINTEDDDEDDDDDDNSDEEDNDDSDDDDDE corresponding to amino acids 79-399 of CAQ2_HUMAN (SEQ ID NO:83), which also corresponds to amino acids 126-446 of N56180_P4 (SEQ ID NO:85), wherein said first amino acid sequence, second amino acid sequence and third amino acid sequence are contiguous and in a sequential order.

2. An isolated polypeptide encoding for an edge portion of N56180_P4 (SEQ ID NO:85), comprising an amino acid sequence being at least 70%, optionally at least about 80%, preferably at least about 85%, more preferably at least about 90% and most preferably at least about 95% homologous to the sequence encoding for HWQISQWWLHFQTPREEGKMKLLELSESADGAAWKRWGGNSNTHRIQ (SEQ ID NO: 629), corresponding to N56180_P4 (SEQ ID NO:85).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The glycosylation sites of variant protein N56180_P4 (SEQ ID NO:85), as compared to the known protein Calsequestrin, cardiac muscle isoform precursor (SEQ ID NO: 83), are described in Table 16 (given according to their position(s) on the amino acid sequence in the first column; the second column indicates whether the glycosylation site is present in the variant protein; and the last column indicates whether the position is different on the variant protein).

TABLE 16
Glycosylation site(s)

| Position(s) on known | Present in | Position in |
|---|---|---|
| amino acid sequence | variant protein? | variant protein? |
| 335 | Yes | 382 |

The variant protein has the following domains, as determined by using InterPro. The domains are described in Table 17:

TABLE 17
InterPro domain(s)

| | | Analysis | |
|---|---|---|---|
| InterPro ID | Domain description | type | Position(s) on protein |
| IPR001393 | Calsequestrin | FPrintScan | 20-43, 47-76 |
| IPR001393 | Calsequestrin | HMMPfam | 124-428, 2-78 |
| IPR001393 | Calsequestrin | ScanRegExp | 20-34 |
| IPR001393 | Calsequestrin | ScanRegExp | 404-423 |

Variant protein N56180_P4 (SEQ ID NO:85) is encoded by transcript N56180_T3 (SEQ ID NO:55). The coding portion of transcript N56180_T3 (SEQ ID NO:55) starts at position 242 and ends at position 1579.

Variant protein N56180_P5 (SEQ ID NO:86) according to the present invention has an amino acid sequence; it is encoded by transcript(s) N56180_T4 (SEQ ID NO:56). An alignment is given to the known protein (Calsequestrin, cardiac muscle isoform precursor (SEQ ID NO: 83)). One or more alignments to one or more previously published protein sequences are given in the alignment table located on the attached CDROM. A brief description of the relationship of the variant protein according to the present invention to each such aligned protein is as follows:

Comparison report between N56180_P5 (SEQ ID NO:86) and CAQ2_HUMAN (SEQ ID NO:83):

1. An isolated chimeric polypeptide encoding for N56180_P5 (SEQ ID NO:86), comprising a first amino acid sequence being at least 90% homologous to MKRTHLFIVGIYFLSSCRAEEGLNFPTYDGKDRVVSLSEKNFKQVLKKYDLLCLYYHEPVSSDKVT QKQFQLKEIVLELVAQVLEHKAIGFVMVDAKKEAKLAKKLGFDEEGSLYILKGDRTIEFDGEFAA DVLVEFLLD corresponding to amino acids 1-140 of CAQ2_HUMAN (SEQ ID NO:83), which also corresponds to amino acids 1-140 of N56180_P5 (SEQ ID NO:86), and a second amino acid sequence being at least 90% homologous to VAKKLSLKMNEVDFYEPFMDEPIAIPNKPYTEEELVEFVKEHQRPTLRRLRPEEMFETWEDDLNGI HIVAFAEKSDPDGYEFLEILKQVARDNTDNPDLSILWIDPDDFPLLVAYWEKTFKIDLFRPQIGVVN VTDADSVWMEIPDDDDLPTAEELEDWIEDVLSGKINTEDDDEDDDDDDNSDEEDNDDSDDDDDE corresponding to amino acids 203-399 of CAQ2_HUMAN (SEQ ID NO:83), which also corresponds to amino acids 141-337 of N56180_P5 (SEQ ID NO:86), wherein said first amino acid sequence and second amino acid sequence are contiguous and in a sequential order.

2. An isolated chimeric polypeptide encoding for an edge portion of N56180_P5 (SEQ ID NO:86), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise DV, having a structure as follows: a sequence starting from any of amino acid numbers 140−x to 140; and ending at any of amino acid numbers 141+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The glycosylation sites of variant protein N56180_P5 (SEQ ID NO:86), as compared to the known protein Calsequestrin, cardiac muscle isoform precursor (SEQ ID NO: 83), are described in Table 18 (given according to their position(s) on the amino acid sequence in the first column; the second column indicates whether the glycosylation site is present in the variant protein; and the last column indicates whether the position is different on the variant protein).

TABLE 18
Glycosylation site(s)

| Position(s) on known | Present in | Position in |
|---|---|---|
| amino acid sequence | variant protein? | variant protein? |
| 335 | Yes | 273 |

The variant protein has the following domains, as determined by using InterPro. The domains are described in Table 19:

TABLE 19
InterPro domain(s)

| | | Analysis | |
|---|---|---|---|
| InterPro ID | Domain description | type | Position(s) on protein |
| IPR001393 | Calsequestrin | FPrintScan | 113-142, 20-43, 47-76, |
| | | | 77-106 |
| IPR001393 | Calsequestrin | HMMPfam | 141-319, 2-140 |
| IPR001393 | Calsequestrin | ScanRegExp | 20-34 |
| IPR001393 | Calsequestrin | ScanRegExp | 295-314 |

Variant protein N56180_P5 (SEQ ID NO:86) is encoded by transcript N56180_T4 (SEQ ID NO:56). The coding portion of transcript N56180_T4 (SEQ ID NO:56) starts at position 242 and ends at position 1252.

Variant protein N56180_P6 (SEQ ID NO:87) according to the present invention has an amino acid sequence; it is encoded by transcript(s) N56180_T5 (SEQ ID NO:57). An alignment is given to the known protein (Calsequestrin, cardiac muscle isoform precursor (SEQ ID NO: 83)). One or more alignments to one or more previously published protein sequences are given in the alignment table located on the attached CDROM. A brief description of the relationship of the variant protein according to the present invention to each such aligned protein is as follows:

Comparison report between N56180_P6 (SEQ ID NO:87) and CAQ2_HUMAN (SEQ ID NO:83):

1. An isolated chimeric polypeptide encoding for N56180_P6 (SEQ ID NO:87), comprising a first amino acid sequence being at least 70%, optionally at least 80%, preferably at least 85%, more preferably at least 90% and most preferably at least 95% homologous to a polypeptide having the sequence NETEAEQSYV (SEQ ID NO: 631) corresponding to amino acids 1-10 of N56180_P6 (SEQ ID NO:87), a second amino acid sequence being at least 90% homologous to RAEEGLNFPTYDGKDRVVSLSEKNFKQVLKKYDLLCLYYHEPVSSDKVTQKQFQLKEIVLELVAQ VLEHKAIGFVMVDAKKEAKLAKKL corresponding to amino acids 18-106 of CAQ2_HUMAN (SEQ ID NO:83), which also corresponds to amino acids 11-99 of N56180_P6 (SEQ ID NO:87), a third amino acid sequence bridging amino acid sequence comprising of D, and a fourth amino acid sequence being at least 90% homologous to YKAFEEAAEHFQPYIKFFATFDKGVAKKLSLKMNEVDFYEPFMDEPIAIPNKPYTEEELVEFVKEH QRPTLRRLRPEEMFETWEDDLNGIHIVAFAEKSDPDGYEFLEILKQVARDNTDNPDLSILWIDPDDF PLLVAYWEKTFKIDLFRPQIGVVNVTDADSVWMEIPDDDDLPTAEELEDWIEDVLSGKINTEDDDE DDDDDDNSDEEDNDDSDDDDDE corresponding to amino acids 179-399 of CAQ2_HUMAN (SEQ ID NO:83), which also corresponds to amino acids 101-321 of N56180_P6 (SEQ ID NO:87), wherein said first amino acid sequence, second amino acid sequence, third amino acid sequence and fourth amino acid sequence are contiguous and in a sequential order.

2. An isolated polypeptide encoding for a head of N56180_P6 (SEQ ID NO:87), comprising a polypeptide being at least 70%, optionally at least about 80%, preferably at least about 85%, more preferably at least about 90% and most preferably at least about 95% homologous to the sequence NETEAEQSYV (SEQ ID NO: 631) of N56180_P6 (SEQ ID NO:87).

3. An isolated polypeptide encoding for an edge portion of N56180_P6 (SEQ ID NO:87), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise LDY having a structure as follows (numbering according to N56180_P6 (SEQ ID NO:87)): a sequence starting from any of amino acid numbers 99−x to 99; and ending at any of amino acid numbers 101+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: unknown. The protein localization is believed to be unknown because of manual inspection of known protein localization and/or gene structure.

The glycosylation sites of variant protein N56180_P6 (SEQ ID NO:87), as compared to the known protein Calsequestrin, cardiac muscle isoform precursor (SEQ ID NO: 83), are described in Table 20 (given according to their position(s) on the amino acid sequence in the first column; the second column indicates whether the glycosylation site is present in the variant protein; and the last column indicates whether the position is different on the variant protein).

TABLE 20
Glycosylation site(s)

| Position(s) on known | | Position in |
|---|---|---|
| amino acid sequence | variant protein? | variant protein? |
| 335 | Yes | 257 |

The variant protein has the following domains, as determined by using InterPro. The domains are described in Table 21:

TABLE 21
InterPro domain(s)

| | | Analysis | |
|---|---|---|---|
| InterPro ID | Domain description | type | Position(s) on protein |
| IPR001393 | Calsequestrin | FPrintScan | 13-36, 40-69, 70-99 |
| IPR001393 | Calsequestrin | HMMPfam | 10-99, 101-303 |
| IPR001393 | Calsequestrin | ScanRegExp | 13-27 |
| IPR001393 | Calsequestrin | ScanRegExp | 279-298 |

Variant protein N56180_P6 (SEQ ID NO:87) is encoded by the following transcript(s): N56180_T5 (SEQ ID NO:57). The coding portion of transcript N56180_T5 (SEQ ID NO:57) starts at position 1 and ends at position 964.

Variant protein N56180_P7 (SEQ ID NO:88) according to the present invention has an amino acid sequence; it is encoded by transcript(s) N56180_T6 (SEQ ID NO:58). An alignment is given to the known protein (Calsequestrin, cardiac muscle isoform precursor (SEQ ID NO: 83)). One or more alignments to one or more previously published protein sequences are given in the alignment table located on the attached CDROM. A brief description of the relationship of the variant protein according to the present invention to each such aligned protein is as follows:

Comparison report between N56180_P7 (SEQ ID NO:88) and CAQ2_HUMAN (SEQ ID NO:83):

1. An isolated chimeric polypeptide encoding for N56180_P7 (SEQ ID NO:88), comprising a first amino acid sequence being at least 70%, optionally at least 80%, preferably at least 85%, more preferably at least 90% and most preferably at least 95% homologous to a polypeptide having the sequence MSSWLSAGSPSSLSV (SEQ ID NO: 632) corresponding to amino acids 1-15 of N56180_P7 (SEQ ID NO:88), and a second amino acid sequence being at least 90% homologous to VAKKLSLKMNEVDFYEPFMDEPIAIPNKPYTEEELVEFVKEHQRPTLRRLRPEEMFETWEDDLNGI HIVAFAEKSDPDGYEFLEILKQVARDNTDNPDLSILWIDPDDFPLLVAYWEKTFKIDLFRPQIGVVN VTDADSVWMEIPDDDDLPTAEELEDWIEDVLSGKINTEDDDEDDDDDDNSDEEDNDDSDDDDDE corresponding to amino acids 203-399 of CAQ2_HUMAN (SEQ ID NO:83), which also corresponds to amino acids 16-212 of N56180_P7 (SEQ ID NO:88), wherein said first amino acid sequence and second amino acid sequence are contiguous and in a sequential order.

2. An isolated polypeptide encoding for a head of N56180_P7 (SEQ ID NO:88), comprising a polypeptide being at least 70%, optionally at least about 80%, preferably at least about 85%, more preferably at least about 90% and most preferably at least about 95% homologous to the sequence MSSWLSAGSPSSLSV (SEQ ID NO: 632) of N56180_P7 (SEQ ID NO:88).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

The glycosylation sites of variant protein N56180_P7 (SEQ ID NO:88), as compared to the known protein Calsequestrin, cardiac muscle isoform precursor (SEQ ID NO: 83), are described in Table 22 (given according to their position(s) on the amino acid sequence in the first column; the second column indicates whether the glycosylation site is present in the variant protein; and the last column indicates whether the position is different on the variant protein).

TABLE 22
Glycosylation site(s)

| Position(s) on known | Present in | Position in |
|---|---|---|
| amino acid sequence | variant protein? | variant protein? |
| 335 | Yes | 148 |

The variant protein has the following domains, as determined by using InterPro. The domains are described in Table 23:

TABLE 23
InterPro domain(s)

| | Domain | | |
|---|---|---|---|
| InterPro ID | description | Analysis type | Position(s) on protein |
| IPR001393 | Calsequestrin | FPrintScan | 130-158, 161-189, 28-55, |
| | | | 61-88 |
| IPR001393 | Calsequestrin | HMMPfam | 16-194 |
| IPR001393 | Calsequestrin | ScanRegExp | 170-189 |

Variant protein N56180_P7 (SEQ ID NO:88) is encoded by the following transcript(s): N56180_T6 (SEQ ID NO:58). The coding portion of transcript N56180_T6 (SEQ ID NO:58) starts at position 71 and ends at position 706.

Variant protein N56180_P8 (SEQ ID NO:89) according to the present invention has an amino acid sequence; it is encoded by transcript(s) N56180_T7 (SEQ ID NO:59). An alignment is given to the known protein (Calsequestrin, cardiac muscle isoform precursor (SEQ ID NO: 83)). One or more alignments to one or more previously published protein sequences are given in the alignment table located on the attached CDROM. A brief description of the relationship of the variant protein according to the present invention to each such aligned protein is as follows:

Comparison report between N56180_P8 (SEQ ID NO:89) and CAQ2_HUMAN (SEQ ID NO:83):

1. An isolated chimeric polypeptide encoding for N56180_P8 (SEQ ID NO:89), comprising a first amino acid sequence being at least 70%, optionally at least 80%, preferably at least 85%, more preferably at least 90% and most preferably at least 95% homologous to a polypeptide having the sequence MCRGYSTLLNPVS (SEQ ID NO: 633) corresponding to amino acids 1-13 of N56180_P8 (SEQ ID NO:89), and a second amino acid sequence being at least 90% homologous to DGYEFLEILKQVARDNTDNPDLSILWIDPDDFPLLVAYWEKTFKIDLFRPQIGVVNVTDADSVWME IPDDDDLPTAEELEDWIEDVLSGKINTEDDDEDDDDDDNSDEEDNDDSDDDDDE corresponding to amino acids 280-399 of CAQ2_HUMAN (SEQ ID NO:83), which also corresponds to amino acids 14-133 of N56180_P8 (SEQ ID NO:89), wherein said first amino acid sequence and second amino acid sequence are contiguous and in a sequential order.

2. An isolated polypeptide encoding for a head of N56180_P8 (SEQ ID NO:89), comprising a polypeptide being at least 70%, optionally at least about 80%, preferably at least about 85%, more preferably at least about 90% and most preferably at least about 95% homologous to the sequence MCRGYSTLLNPVS (SEQ ID NO: 633) of N56180_P8 (SEQ ID NO:89).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

The glycosylation sites of variant protein N56180_P8 (SEQ ID NO:89), as compared to the known protein Calsequestrin, cardiac muscle isoform precursor (SEQ ID NO: 83), are described in Table 24 (given according to their position(s) on the amino acid sequence in the first column; the second column indicates whether the glycosylation site is present in the variant protein; and the last column indicates whether the position is different on the variant protein).

TABLE 24
Glycosylation site(s)

| Position(s) on known | Present in | Position in |
|---|---|---|
| amino acid sequence | variant protein? | variant protein? |
| 335 | Yes | 69 |

The variant protein has the following domains, as determined by using InterPro. The domains are described in Table 25:

TABLE 25
InterPro domain(s)

| | | Analysis | |
|---|---|---|---|
| InterPro ID | Domain description | type | Position(s) on protein |
| IPR001393 | Calsequestrin | FPrintScan | 51-79, 82-110 |
| IPR001393 | Calsequestrin | HMMPfam | 14-115 |
| IPR001393 | Calsequestrin | ScanRegExp | 91-110 |

Variant protein N56180_P8 (SEQ ID NO:89) is encoded by the following transcript(s): N56180_T7 (SEQ ID NO:59). The coding portion of transcript N56180_T7 (SEQ ID NO:59) starts at position 97 and ends at position 495.

Variant protein N56180_P9 (SEQ ID NO:90) according to the present invention has an amino acid sequence; it is encoded by transcript(s) N56180_T8 (SEQ ID NO:60). An alignment is given to the known protein (Calsequestrin, cardiac muscle isoform precursor (SEQ ID NO: 83)). One or more alignments to one or more previously published protein sequences are given in the alignment table located on the attached CDROM. A brief description of the relationship of the variant protein according to the present invention to each such aligned protein is as follows:

Comparison report between N56180_P9 (SEQ ID NO:90) and CAQ2_HUMAN (SEQ ID NO:83):

1. An isolated chimeric polypeptide encoding for N56180_P9 (SEQ ID NO:90), comprising a first amino acid sequence being at least 90% homologous to MKRTHLFIVGIYFLSSCRAEEGLNFPTYDGKDRVVSLSEKNFKQVLKKYDLLCLYYHEPVSSDKVT QKQFQLKEIVLELVAQVLEHKAIGFVMVDAKKEAKLAKKLGFDEEGSLYILKGDRTIEFDGEFAA DVLVEFLLDLIEDPVEIISSKLEVQAFERIEDYIKLIGFFKSEDSEYYKAFEEAAEHFQPYIKFFATFD KGVAKKLSLKMNEVDFYEPFMDEPIAIPNKPYTEEELVEFVKEHQR corresponding to amino acids 1-246 of CAQ2_HUMAN (SEQ ID NO:83), which also corresponds to amino acids 1-246 of N56180_P9 (SEQ ID NO:90), and a second amino acid sequence being at least 70%, optionally at least 80%, preferably at least 85%, more preferably at least 90% and most preferably at least 95% homologous to a polypeptide having the sequence SRNWTQ (SEQ ID NO: 634) corresponding to amino acids 247-252 of N56180_P9 (SEQ ID NO:90), wherein said first amino acid sequence and second amino acid sequence are contiguous and in a sequential order.

2. An isolated polypeptide encoding for a tail of N56180_P9 (SEQ ID NO:90), comprising a polypeptide being at least 70%, optionally at least about 80%, preferably at least about 85%, more preferably at least about 90% and most preferably at least about 95% homologous to the sequence SRNWTQ (SEQ ID NO: 634) in N56180_P9 (SEQ ID NO:90).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The glycosylation sites of variant protein N56180_P9 (SEQ ID NO:90), as compared to the known protein Calsequestrin, cardiac muscle isoform precursor (SEQ ID NO: 83), are described in Table 26 (given according to their position(s) on the amino acid sequence in the first column; the second column indicates whether the glycosylation site is present in the variant protein; and the last column indicates whether the position is different on the variant protein).

TABLE 26
Glycosylation site(s)

| Position(s) on known | Present | |
|---|---|---|
| amino acid sequence | in variant protein? | Position in variant protein? |
| 335 | No | |

The variant protein has the following domains, as determined by using InterPro. The domains are described in Table 27:

TABLE 27
InterPro domain(s)

| | Domain | | |
|---|---|---|---|
| InterPro ID | description | Analysis type | Position(s) on protein |
| IPR001393 | Calsequestrin | FPrintScan | 113-142, 154-183, |
| | | | 184-213, 20-43, |
| | | | 215-242, 47-76, 77-106 |
| IPR001393 | Calsequestrin | HMMPfam | 2-247 |
| IPR001393 | Calsequestrin | ScanRegExp | 20-34 |

Variant protein N56180_P9 (SEQ ID NO:90) is encoded by the following transcript(s): N56180_T8 (SEQ ID NO:60). The coding portion of transcript N56180_T8 (SEQ ID NO:60) starts at position 242 and ends at position 997.

Table 28 below describes the starting and ending position of N56180_node_{—}6 (SEQ ID NO:63) on each of the relevant transcripts. Experimental results for this segment are described below.

TABLE 28
Segment location on transcripts

| | Segment | Segment |
|---|---|---|
| Transcript name | starting position | ending position |
| N56180_T3 (SEQ ID NO: 55) | 476 | 616 |

Table 29 below describes the starting and ending position of N56180_node_{—}22 (SEQ ID NO:65) on each of the relevant transcripts. Experimental results for this segment are described below.

TABLE 29
Segment location on transcripts

| | Segment | Segment |
|---|---|---|
| Transcript name | starting position | ending position |
| N56180_T8 (SEQ ID NO: 60) | 979 | 1259 |

Table 30 below describes the starting and ending position of N56180_node_{—}33 (SEQ ID NO:81) on each of the relevant transcripts. Experimental results for this segment are described below.

TABLE 30
Segment location on transcripts

| | Segment | Segment |
|---|---|---|
| Transcript name | starting position | ending position |
| N56180_T1 (SEQ ID NO: 54) | 1351 | 1396 |
| N56180_T3 (SEQ ID NO: 55) | 1397 | 1442 |
| N56180_T4 (SEQ ID NO: 56) | 1070 | 1115 |
| N56180_T5 (SEQ ID NO: 57) | 782 | 827 |
| N56180_T6 (SEQ ID NO: 58) | 524 | 569 |
| N56180_T7 (SEQ ID NO: 59) | 313 | 358 |

Table 31 below describes the starting and ending position of N56180_node_{—}34 (SEQ ID NO:67) on each of the relevant transcripts. Experimental results for this segment are described below.

TABLE 31
Segment location on transcripts

| | Segment | Segment |
|---|---|---|
| Transcript name | starting position | ending position |
| N56180_T1 (SEQ ID NO: 54) | 1397 | 1644 |
| N56180_T3 (SEQ ID NO: 55) | 1443 | 1690 |
| N56180_T4 (SEQ ID NO: 56) | 1116 | 1363 |
| N56180_T5 (SEQ ID NO: 57) | 828 | 1075 |
| N56180_T6 (SEQ ID NO: 58) | 570 | 817 |
| N56180_T7 (SEQ ID NO: 59) | 359 | 606 |

Expression of Homo sapiens calsequestrin 2 (cardiac muscle) (CASQ2) N56180 transcripts which are detectable by amplicon as depicted in sequence name N56180 seg33-34 (SEQ ID NO:93) specifically in heart tissue:

Expression of Homo sapiens calsequestrin 2 (cardiac muscle) (CASQ2) transcripts detectable by or according to seg33-34, N56180 seg33-34 (SEQ ID NO:93) amplicon and primers N56180 seg33-34F (SEQ ID NO: 91) and N56180 seg33-34R (SEQ ID NO:92) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44-46, Table 7, above, “Tissue samples in normal panel”), to obtain a value of expression for each sample relative to median of the heart tissue.

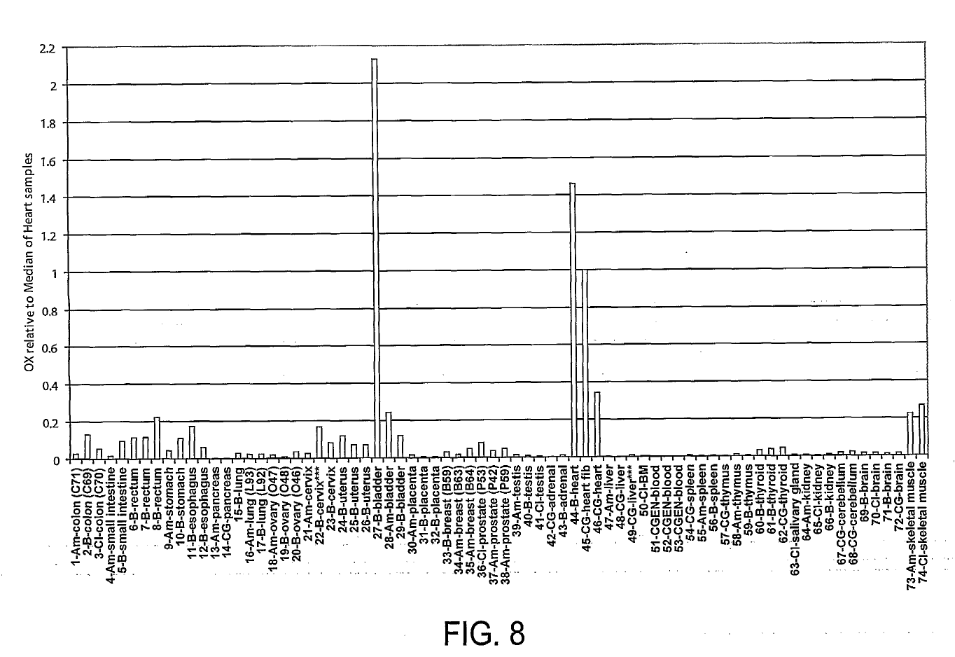

FIG. 8 is a histogram showing relative expression of the above-indicated Homo sapiens calsequestrin 2 (cardiac muscle) (CASQ2) transcripts in heart tissue samples as opposed to other tissues.

As is evident from FIG. 8, the expression of Homo sapiens calsequestrin 2 (cardiac muscle) (CASQ2) transcripts detectable by the above amplicon in heart tissue samples was significantly higher than in most of the other samples (Sample Nos. 1-26, 28-43, 47-74 Table 7, “Tissue samples in normal panel”).

Primer pairs are also optionally and preferably encompassed within the present invention; for example, for the above experiment, the following primer pair was used as a non-limiting illustrative example only of a suitable primer pair: N56180 seg33-34F (SEQ ID NO: 91) forward primer; and N56180 seg33-34R (SEQ ID NO:92) reverse primer.

The present invention also preferably encompasses any amplicon obtained through the use of any suitable primer pair; for example, for the above experiment, the following amplicon was obtained as a non-limiting illustrative example only of a suitable amplicon: N56180 seg33-34 (SEQ ID NO:93).

Primers:

Forward primer N56180 seg33-34F (SEQ ID NO:91):
CTGGATTGAGGATGTGCTTTCTG
Reverse primer N56180 seg33-34R (SEQ ID NO:92):
TTTGGAGTTGGGCTATTCATCAT
Amplicon N56180 seg33-34 (SEQ ID NO:93):
CTGGATTGAGGATGTGCTTTCTGGAAAGATAAACACTGAAGATGATGATG
AAGATGATGATGATGATGATAATTCTGATGAAGAGGATAATGATGACAGT
GATGACGATGATGATGAATAGCCCAACTCCAAA

Expression of Calsequestrin, cardiac muscle isoform transcripts which are detectable by amplicon which are detectable by amplicon as depicted in sequence name N56180seg22 (SEQ ID NO: 96) specifically in heart tissue:

Expression of Calsequestrin, cardiac muscle isoform transcripts detectable by or according to seg 22 node, N56180 amplicon and N56180 seg22F (SEQ ID NO: 94) and N56180 seg22R (SEQ ID NO:95) primers was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44, 45, 46, Table 7, above, “Tissue samples in normal panel”), to obtain a value of expression for each sample relative to median of the heart tissue.

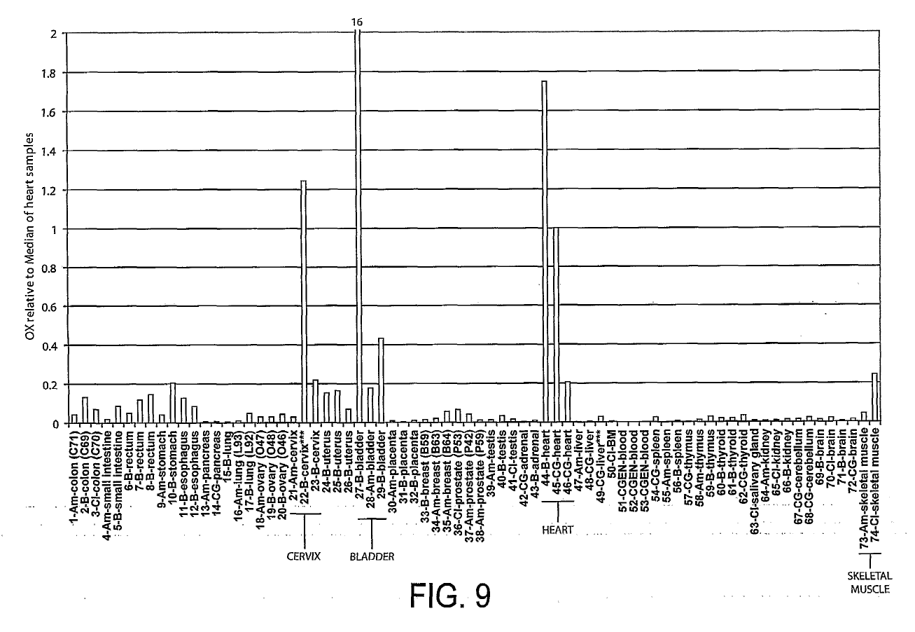

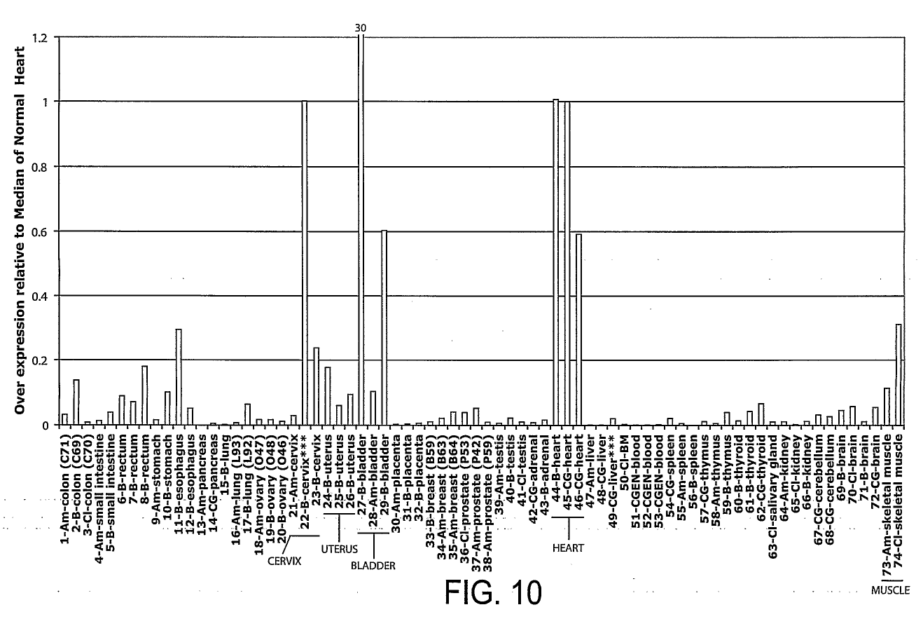

FIG. 9 is a histogram showing specific expression of the above-indicated Calsequestrin, cardiac muscle isoform transcripts in heart tissue samples as opposed to other tissues. As is evident from FIG. 9, the expression of Calsequestrin, cardiac muscle isoform transcripts detectable by the above amplicon in heart tissue samples was significantly higher than in most of the other samples (non-heart tissue sample Nos. 1-21, 23-26, 28-43, 47-74 Table 7, “Tissue samples in normal panel”).

Primer pairs are also optionally and preferably encompassed within the present invention; for example, for the above experiment, the following primer pair was used as a non-limiting illustrative example only of a suitable primer pair: N56180 seg22F (SEQ ID NO: 94) forward primer; and N56180 seg 22R (SEQ ID NO:95) reverse primer.

The present invention also preferably encompasses any amplicon obtained through the use of any suitable primer pair; for example, for the above experiment, the following amplicon was obtained as a non-limiting illustrative example only of a suitable amplicon: N56180 seg22 (SEQ ID NO: 96).

N56180 seg22F (SEQ ID NO:94):
TTGATACCACTTAGTGTAGCTCCAGC
N56180 seg22R (SEQ ID NO:95) (SEQ ID NO:337):
TCAAGTAGTTGCTACAGACGCCA
N56180 seg22 (SEQ ID NO:96):
TTGATACCACTTAGTGTAGCTCCAGCATGGATCAGCAAACTTTTTCTGTA
AAGAACAAAATGGTAAATATTTCAGGTTCTGTGGGCCAGATGGCGTCTGT
AGCAACTACTTGA

Expression of Calsequestrin, cardiac muscle isoformtranscripts which are detectable by amplicon which are detectable by amplicon as depicted in sequence name N56180seg6 (SEQ ID NO: 99) specifically in heart tissue:

Expression of Calsequestrin, cardiac muscle isoform transcripts detectable by or according to seg6, N56180seg6 (SEQ ID NO: 99) amplicon and N56180 seg6F (SEQ ID NO:97) and N56180 seg6R (SEQ ID NO:98) primers was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44, 45, 46, Table 7, above, “Tissue samples in normal panel”), to obtain a value of expression for each sample relative to median of the heart tissue.

FIG. 10 is a histogram showing specific expression of the above-indicated Calsequestrin, cardiac muscle isoform transcripts in heart tissue samples as opposed to other tissues. As is evident from FIG. 10, the expression of Calsequestrin, cardiac muscle isoform transcripts detectable by the above amplicon in heart tissue samples was significantly higher than in most other samples (non-heart tissue sample Nos. 1-21, 23-26, 28, 30-43 47-74 Table 7 above, “Tissue samples in normal panel”).

Primer pairs are also optionally and preferably encompassed within the present invention; for example, for the above experiment, the following primer pair was used as a non-limiting illustrative example only of a suitable primer pair: N56180 seg6F (SEQ ID NO:97) forward primer; and N56180 seg6R (SEQ ID NO:98) reverse primer.

The present invention also preferably encompasses any amplicon obtained through the use of any suitable primer pair; for example, for the above experiment, the following amplicon was obtained as a non-limiting illustrative example only of a suitable amplicon: N56180 seg6 (SEQ ID NO:99).

N56180 seg6F (SEQ ID NO:97):
ATATCCCAGTGGTGGTTGCATT
N56180 seg6R (SEQ ID NO:98):
CCCTCCCCAGCGTTTCC
N56180 seg6 (SEQ ID NO:99):
ATATCCCAGTGGTGGTTGCATTTCCAAACCCCAAGAGAGGAAGGCAAAAT
GAAGTTGCTGGAGTTGAGTGAATCTGCAGATGGAGCTGCGTGGAAACGCT
GGGGAGGG

### Description for Cluster S67314

Cluster S67314 features 4 transcript(s) and 8 segment(s) of interest, the names for which are given in Tables 32 and 33, respectively. The selected protein variants are given in table 34.

TABLE 32
Transcripts of interest
Transcript Name

S67314_PEA_1_T4 (SEQ ID NO: 100)
S67314_PEA_1_T5 (SEQ ID NO: 101)
S67314_PEA_1_T6 (SEQ ID NO: 102)
S67314_PEA_1_T7 (SEQ ID NO: 103)

TABLE 33
Segments of interest

| Segment Name | Corresponding Transcript(s) |
|---|---|
| S67314_PEA_1_node_0 | S67314_PEA_1_T4 (SEQ ID NO: 100), |
| (SEQ ID NO: 104) | S67314_PEA_1_T5 (SEQ ID NO: 101), |
| | S67314_PEA_1_T6 (SEQ ID NO: 102) |
| | and |
| | S67314_PEA_1_T7 (SEQ ID NO: 103) |
| S67314_PEA_1_node_4 | S67314_PEA_1_T4 (SEQ ID NO: 100), |
| (SEQ ID NO: 105) | S67314_PEA_1_T5 (SEQ ID NO: 101), |
| | S67314_PEA_1_T6 (SEQ ID NO: 102) |
| | and |
| | S67314_PEA_1_T7 (SEQ ID NO: 103) |
| S67314_PEA_1_node_11 | S67314_PEA_1_T4 (SEQ ID NO: 100) |
| (SEQ ID NO: 106) | |
| S67314_PEA_1_node_13 | S67314_PEA_1_T7 (SEQ ID NO: 103) |
| (SEQ ID NO: 107) | |
| S67314_PEA_1_node_15 | S67314_PEA_1_T5 (SEQ ID NO: 101) |
| (SEQ ID NO: 108) | |
| S67314_PEA_1_node_17 | S67314_PEA_1_T6 (SEQ ID NO: 102) |
| (SEQ ID NO: 109) | |
| S67314_PEA_1_node_3 | S67314_PEA_1_T7 (SEQ ID NO: 103) |
| (SEQ ID NO: 110) | |
| S67314_PEA_1_node_10 | S67314_PEA_1_T4 (SEQ ID NO: 100), |
| (SEQ ID NO: 111) | S67314_PEA_1_T5 (SEQ ID NO: 101), |
| | S67314_PEA_1_T6 (SEQ ID NO: 102) |
| | and |
| | S67314_PEA_1_T7 (SEQ ID NO: 103) |

TABLE 34
Proteins of interest

| Protein Name | Corresponding Transcript(s) |
|---|---|
| S67314_PEA_1_P4 | S67314_PEA_1_T4 (SEQ ID NO: 100) |
| (SEQ ID NO: 114) | |
| S67314_PEA_1_P5 | S67314_PEA_1_T5 (SEQ ID NO: 101) |
| (SEQ ID NO: 115) | |
| S67314_PEA_1_P6 | S67314_PEA_1_T6 (SEQ ID NO: 102) |
| (SEQ ID NO: 116) | |
| S67314_PEA_1_P7 | S67314_PEA_1_T7 (SEQ ID NO: 103) |
| (SEQ ID NO: 117) | |

These sequences are variants of the known protein Fatty acid-binding protein (SEQ ID NO:112), heart (SwissProt accession identifier FABH_HUMAN (SEQ ID NO:112); known also according to the synonyms H-FABP; Muscle fatty acid-binding protein; M-FABP; Mammary-derived growth inhibitor; MDGI), referred to herein as the previously known protein.

Protein Fatty acid-binding protein (SEQ ID NO:112), heart is known or believed to have the following function(s): FABP are thought to play a role in the intracellular transport of long-chain fatty acids and their acyl-CoA esters. Known polymorphisms for this sequence are as shown in Table 35.

TABLE 35
Amino acid mutations for Known Protein

| SNP position(s) on amino | |
|---|---|
| acid sequence | Comment |
| 1 | V -> A |
| 104 | L -> K |
| 124 | C -> S |
| 129 | E -> Q |

Protein Fatty acid-binding protein (SEQ ID NO:112), heart localization is believed to be Cytoplasmic.

The following GO Annotation(s) apply to the previously known protein. The following annotation(s) were found: negative control of cell proliferation, which are annotation(s) related to Biological Process; and lipid binding, which are annotation(s) related to Molecular Function.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of S67314) may optionally have one or more of the following utilities, as described with regard to the Table 36 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 36
Utilities for Variants of S67314, related to Fatty acid-binding protein (SEQ ID NO: 112)
or FABP: FABPH_HUMAN

| | | |
|---|---|---|
| 1. horses with colic | Determination of I-FABP concentrations in | Nieto JE et al.,, Am J |
| | abdominal fluid and plasma may be useful | Vet Res. 2005 |
| | for predicting survival and the need for | Feb; 66(2): 223-32 |
| | abdominal surgical intervention in horses | |
| | with colic. Furthermore, serum creatine | |
| | kinase activity and color and protein | |
| | concentrations of abdominal fluid may be | |
| | useful in the diagnosis of intestinal ischemia | |
| 3. novel marker for the diagnosis | increased serum H-FABP levels in patients | Arimoto T et al., J |
| of congestive heart failure | with congestive heart failure | Card Fail. 2005 |
| | | Feb; 11(1): 56-60 |
| 4. biomarker of early acute | plasma levels of H-FABP were measured by | Chen L et al. J |
| myocardial infarction | sandwich ELISA in 93 patients with | Huazhong Univ Sci |
| | suspected AMI at admission within 6 h after | Technolog Med Sci. |
| | onset. These results revealed that H-FABP | 2004; 24(5): 449-51, |
| | possessed high diagnostic sensitivity and | 459 |
| | specificity for AMI in early stage, especially | |
| | within 3 h after onset of persistent angina | |
| | pectoris | |
| 5. detection of acute myocardial | abundant in heart and has low | Alhadi HA et al., |
| infarction (AMI) | concentrations in the blood and in tissues | QJM. 2004 |
| | outside the heart. It appears in the blood as | Apr; 97(4): 187-98 |
| | early as 1.5 h after onset of symptoms of | |
| | infarction, peaks around 6 h and returns to | |
| | baseline values in 24 h | |
| 6. diagnosis of acute myocardial | a simple one-step immunochromatography | Watanabe T et al., |
| infarction (AMI) | technique to detect H-FABP in whole blood | Clin Biochem. 2001 |
| | sample. Further studies are required to | Jun; 34(4): 257-63 |
| | identify the value of this point-of-care | |
| | testing (POCT) as a diagnostic marker for | |
| | AMI. | |
| 7. clinical validity of H-FABP as | H-FABP is a low molecular weight | Okamoto F et al., |
| a biochemical diagnostic marker | cytoplasmic protein and present abundantly | Clin Chem Lab Med. |
| in the early phase of acute | in the myocardium. When the myocardium | 2000 Mar; 38(3): 231-8 |
| myocardial infarction (AMI) | is injured, as in the case of myocardial | |
| | infarction, low molecular weight | |
| | cytoplasmic proteins including H-FABP are | |
| | released into the circulation and H-FABP is | |
| | detectable in a blood sample | |
| 8. plasma markers for detection of | H-FABP not only proves to be an excellent | Pelsers MM et al., |
| tissue injury | early marker for cardiac injury in acute | Clin Chim Acta. |
| | coronary syndromes, but also allows | 2005 Feb; 352(1-2): |
| | detection of minor myocardial injury in | 15-35 |
| | heart failure and unstable angina. | |
| 9. diagnosis of neurodegenerative | H-FABP might be a useful biomarker for | Steinacker P et al., |
| diseases | the differentiation between the dementias | Neurosci Lett. 2004 |
| | examined if levels in CSF and serum are | Nov 3; 370(1): 36-9 |
| | determined in parallel | |
| 10. gastric carcinoma | Immunohistochemistry with anti-H-FABP | Hashimoto T et al., |
| | antibody was performed in 669 gastric | Pathobiology. |
| | carcinomas and 60 tubular adenomas of the | 2004; 71(5): 267-73 |
| | stomach. conclusions: A subset of human | |
| | gastric carcinoma expresses H-FABP and its | |
| | expression is associated with FAS status, | |
| | disease progression, tumor aggressiveness | |
| | and poor patient survival. | |
| 11. chronic congestive heart | Concentration of the marker increase in the | Sato Y et al., Heart. |
| failure | absence of ischemic events in the subset of | 2004 |
| | patients with heart failure whose long term | Oct; 90(10): 1110-3 |
| | outcomes are most adverse | |
| 12. diagnosis of brain damage- | post-mortem cerebrospinal fluid samples | Lescuyer P et al., |
| related disorders including | were used as a model of massive brain insult | Proteomics. 2004 |
| cerebrovascular, dementia, and | to identify new markers potentially relevant | Aug; 4(8): 2234-41 |
| other neurodegenerative diseases. | for neurodegeneration. H-FABP, identified | |
| | in this study, is a potential marker of | |
| | Creutzfeldt-Jakob disease and stroke | |
| 13. Plasma marker for diagnosis | Patient studies indicate that B-FABP and H- | Pelsers MM et al., |
| of minor brain injury | FABP are more sensitive markers for minor | Clin Chem. 2004 |
| | brain injury than the currently used markers | Sep; 50(9): 1568-75. |
| | S100B and NSE | Epub 2004 Jun 24. |
| 14. evaluating the severity of | Serum concentrations of H-FABP were | Goto T et al., |
| congestive heart failure | determined in 48 patients with acute | Heart. 2003 |
| | deterioration of congestive heart failure, | Nov; 89(11): 1303-7 |
| | both before and after effective treatment. | |
| | The decreases in H-FABP concentrations | |
| | after treatment correlated with the decrease | |
| | in BNP concentrations | |
| 15. early diagnosis of stroke | H-FABP appears to be a valid serum | Zimmermann-Ivol |
| | biomarker for the early diagnosis of stroke | CG et al., Mol Cell |
| | | Proteomics. 2004 |
| | | Jan; 3(1): 66-72. Epub |
| | | 2003 Oct 26 |
| 16. diagnosis of Creutzfeldt-Jakob | H-FABP detection could be helpful as a | Guillaume E et al., |
| disease (CJD) | blood screening test for a pre-mortem | Proteomics. 2003 |
| | diagnosis of the disease and also to prevent | Aug; 3(8): 1495-9 |
| | the risk of iatrogenic transmission of CJD | |
| | through blood transfusion. | |
| 17. acute coronary syndrome | H-FABP can be an early diagnostic and | Nakata T. et al., |
| | prognostic biochemical marker, particularly | Cardiology. |
| | within the first 6 h from the onset of chest | 2003; 99(2): 96-104 |
| | symptoms, in patients with chest pain at an | |
| | emergency department. | |
| 19. marker of reperfusion after | (H-FABP) is a small, cytosolic protein | de Lemos JA et al., |
| thrombolytic therapy | found in high concentrations in the | Clin Chim Acta. |
| | myocardium. successful reperfusion can be | 2000 Aug; 298(1-2): |
| | detected within the first 60 min after | 85-97 |
| | thrombolysis with either H-FABP. | |
| 20. Marker for diagnosis of | H-FABP appears rapidly in plasma after | Hayashida N et al., |
| myocardial injury in patients | reperfusion and reaches its peak earlier than | Jpn Circ J. 2000 |
| undergoing cardiac surgery. | other available biochemical markers; it | Jan; 64(1): 18-22 |
| | appears also in urine and the levels | |
| | correlated with cardiac function. Plasma and | |
| | urinary H-FABP may be an early and | |
| | sensitive biochemical marker for the | |
| | diagnosis of myocardial injury in patients | |
| | undergoing cardiac surgery. | |
| 21. H-FABP levels in | expression of H-FABP was markedly | Pu L et al., Mol Cell |
| synaptosomal plasma membranes | reduced in aged mouse brain. | Biochem. 1999 |
| and synaptosomal cytosol may be | | Aug; 198(1-2): 69-78 |
| important factors modulating | | |
| neuronal differentiation and | | |
| function | | |
| 22. genetic markers to improve the | relationship between variation in the heart | Gerbens F. J Anim |
| meat quality of pigs by breeding | fatty acid-binding protein (H-FABP) gene | Sci. 1999 |
| | (FABP3) and intramuscular fat (IMF) | Apr; 77(4): 846-52 |
| | content. | |
| 23. Obesity in women | Increased capacity of intracellular fatty acid | Kempen KP et al., |
| | transport in skeletal muscle cells is involved | Eur J Clin Invest. |
| | in the physiological adaptations of fat | 1998 |
| | metabolism to energy restriction in obese | Dec; 28(12): 1030-7. |
| | female subjects. | |
| 24. Monoclonal antibodies to | FABP is abundantly present in heart and | Roos W et al., J |
| human heart fatty acid-binding | some skeletal muscles, was recently found | Immunol Methods. |
| protein. | to be a useful plasma marker for acute | 1995 Jun |
| | myocardial infarction | 14; 183(1): 149-53 |
| 25. Diabetes | expression of the H-FABP gene in aorta | Sakai K et al., Eur J |
| | may be specifically and dramatically | Biochem. 1995 Apr |
| | suppressed in streptozotocin-diabetic rats, | 1; 229(1): 201-6 |
| | and that this suppression appears to be | |
| | regulated by insulin. | |
| 26. Diagnostic value of heart | Among patients with MI admission FABP | Trifonov IR et al., |
| fatty-acid binding protein in early | compared with admission TnI more | Kardiologiia. |
| hospitalized patients with non ST | frequently exceeded diagnostic level (in 18 | 2003; 43(5): 4-8. |
| elevation acute coronary | vs 9 patients, respectively, p = 0.009). | |
| syndrome | Sensitivity and specificity of admission | |
| | levels of FABP and TnI for diagnosis of MI | |
| | were 58 and 85%, 29% and 100%, | |
| | respectively .FABP can be used as | |
| | additional diagnostic tool for myocardial | |
| | infarction (MI) detection in early admitted | |
| | patients with NSTEACS | |
| 27. Elevated levels of H-FABP | Circulating concentrations of cardiac | Ehrhardt S et al., |
| indicated myocardial impairment | proteins in complicated and uncomplicated | Trop Med Int Health. |
| in complicated but not in | Plasmodium falciparum malaria | 2004 |
| uncomplicated falciparum malaria | | Oct; 9(10): 1099-103 |
| 28. Diagnostic value of heart fatty | Myoglobin and hFABP provide little | Alansari SE et al., |
| acid binding protein and | clinical value when measured on admission | Ann Clin Biochem. |
| myoglobin in patients admitted | in patients presenting with chest pain. | 2004 Sep; 41(Pt |
| with chest pain | | 5): 391-6 |
| 29. H-FABP is decreased in | Aberrant expression of FABPs, especially | Cheon MS et al., J |
| brains of patients with Down | H-FABP may alter membrane fluidity and | Neural Transm |
| syndrome and Alzheimer's | signal transduction, and consequently could | Suppl. |
| disease; these may be measured | be involved in cellular dysfunction in | 2003; (67): 225-34 |
| for example in cerebrospinal fluid | neurodegenerative disorders. | |
| as well as other types of samples | | |
| (such as blood samples for | | |
| example). | | |
| 30. Pericardial fluid level of heart- | H-FABP-a sensitive and specific marker for | Tambara K. Int J |
| type cytoplasmic fatty acid- | the early diagnosis of acute myocardial | Cardiol. 2004 |
| binding protein (H-FABP) is an | infarction. Our hypothesis was that serum or | Feb; 93(2-3): 281-4. |
| indicator of severe myocardial | pericardial fluid levels of H-FABP can | |
| ischemia | reflect not only myocardial infarction but | |
| | also myocardial ischemia pericardial fluid | |
| | reflects pathophysiological conditions of | |
| | cardiomyocytes more sensitively than | |
| | circulating blood. | |
| 31. biomarkers may represent | pre-transplant concentration of these tissue | Gok MA et al., Clin |
| reliable pre-transplant indicators | injury biomarkers determined pre-transplant | Chim Acta. 2003 |
| of immediate kidney viability and | did not correlate with subsequent longer- | Dec; 338(1-2): 33-43 |
| short-term kidney function, they | term renal function | |
| do not predict the efficacy of renal | | |
| function in the longer term | | |
| 33. The value of F/M (ratio of | The concentration of hFABP (F), is | Furuhashi M et al., |
| FABP to myoglobin) after | significantly influenced by renal clearance | Nephron Clin Pract. |
| hemodialysis, but not the | and thus has limitations to its usefulness in | 2003; 93(2): C69-74 |
| concentration of hFABP itself, | patients with renal dysfunction. We | |
| might be a newly useful marker | evaluated whether the serum ratio of hFABP | |
| for estimation of cardiac damage | to myoglobin (F/M) might be a useful | |
| and volume overload in | marker for assessing cardiac damage in | |
| hemodialysis patients | hemodialysis patients | |
| 34. H-FABP, a marker of | Increased concentrations of H-FABP | Setsuta K et al. Circ |
| membrane damage, is related to | significantly correlated with the | J. 2004 |
| activated TNF and the Fas/FasL | concentrations of TNF- alpha and sFas | Aug; 68(8): 747-50 |
| system, which suggests a | independent of renal function | |
| pathophysiological role of | | |
| cardiomyocyte necrosis and/or | | |
| apoptosis in patients with | | |
| worsening heart failure. | | |
| 35. Assessment of coronary | FABP and myoglobin perform equally well | Klapper SR, et al., |
| reperfusion | as reperfusion markers, and successful | Heart. 2001 |
| | reperfusion can be assessed, with positive | Mar; 85(3): 278-85 |
| | predictive values of 87% and 88%, or even | |
| | 97% and 95% when infarct size is also taken | |
| | into account | |
| 36. Estimation of myocardial | FABP released from the heart after AMI is | Glatz JF et al., Br |
| infarct size | quantitatively recovered in plasma and that | Heart J. 1994 |
| | FABP is a useful biochemical plasma | Feb; 71(2): 135-40 |
| | marker for the estimation of myocardial | |
| | infarct size in humans. | |

According to other optional embodiments of the present invention, variants of this cluster according to the present invention (amino acid and/or nucleic acid sequences of S67314) may optionally have one or more of the following utilities, some of which are related to utilities described above. It should be noted that these utilities are optionally and preferably suitable for human and nonhuman animals as subjects, except where otherwise noted.

A non-limiting example of such a utility is the detection, diagnosis and/or determination of cardiac toxicity by an anti-cancer chemotherapeutic agent, particularly an anthracycline-type anticancer chemotherapeutic agent, including but not limited to adriamycin, daunorubicin hydrochloride, epirubicin hydrochloride, idarubicin hydrochloride, pirarubicin hydrochloride, or aclarubicin hydrochloride. The method comprises detecting a S67314 variant, for example a variant protein, protein fragment, peptide, polynucleotide, polynucleotide fragment and/or oligonucleotide as described herein, optionally and preferably in a serum sample. Such detection enables cardiotoxicity caused by the chemotherapeutic agent to be optionally and preferably detected sensitively at an early stage, which for example enables physicians to conduct medical procedures at an early stage of cardiotoxicity expression, such as change of pharmaceutical agents and the like.

Use of the known protein (FABPH or FABP) for detecting cardiotoxicity of anthracycline-like chemotherapeutic agents is described with regard to European Patent Application No. EP1491896, hereby incorporated by reference as if fully set forth herein.

For cancer treatment, prolonged administration of anthracycline-type anticancer chemotherapeutic agents is generally employed. While anthracycline-type anticancer chemotherapeutic agents have a wide range of anticancer spectrum, they are known to show cardiotoxicity as a common side effect due to myocardial injury action.

As a method of determining toxicity to the heart of an anthracycline-type anticancer chemotherapeutic agent, an electrocardiogram analysis, a blood biochemical test comprising measurement of Creatine Kinase (CK) in blood, an echocardiogram analysis and the like, which are general tests of cardiac function, are conventionally known and performed. However, since electrocardiogram analysis and echocardiogram analysis do not specifically detect cardiotoxicity of anthracycline-type anticancer chemotherapeutic agents, they do not have sufficient sensitivity to pick up the initial stage of the onset of toxicity of the agents, and can detect only the advanced cardiotoxicity. In addition, only a small amount of creatine kinase flows (escapes) into the blood due to the cardiotoxicity induced by anthracycline-type anticancer chemotherapeutic agents and creatine kinase requires a long time before escape, and therefore, a problem in clinical situation has existed in that cardiotoxicity of anthracycline-type anticancer chemotherapeutic agents is not precisely reflected.

This EP applications describes a method which involves drawing blood from the patients receiving such chemotherapy, comparing the level of Human H-FABP contained therein with that of human H-FABP contained in the blood of healthy volunteers, and further by comparing the value in patients receiving such therapy with a cut-off value for the determination of acute myocardial infarction, so as to determine the level of toxicity. According to preferred embodiments of the present invention, these levels are compared for S67314 variants so as to be able to distinguish between myocardial infarction and/or other acute cardiovascular event, and cardiotoxicity caused by such chemotherapy.

Optionally and preferably, the “determination of toxicity” includes but is not limited to determination of the presence or absence of expressed toxicity in the heart and determination of the level of toxicity when toxicity is present.

The method according to the present invention for detecting and/or diagnosing cardiotoxicity through detecting one or more S67314 variants may also optionally include one or more known methods for the detection of abnormality in the heart, such as electrocardiogram analysis, echocardiogram analysis and the like, more preferably in combination to determine toxicity. By combining a plurality test methods, the toxicity may optionally be determined more accurately and sensitively.

Detecting S67314 variants optionally and preferably includes quantitative measurement of a level of at least one variant in a tissue sample. Also, detecting optionally and preferably includes performing a plurality of measurements over time, for example to determine the level and progress of cardiotoxicity.

The determination results thus obtained are useful for a physician to decide, for a cancer patient under medication with an anthracycline-type anticancer chemotherapeutic agent, if (1) the administration of the same agent is to be continued, (2) the administration is to be stopped (the kind (type) of anticancer agent is changed) or (3) the dose is to be increased or decreased, and the like, and for a patient for whom administration of an anthracycline-type anticancer chemotherapeutic agent was once stopped due to the expression of cardiotoxicity, it is useful for determining if (4) administration of this agent is to be resumed.

As a specific non-limiting example, during the administration period of a chemotherapeutic agent such as adriamycin, for example, blood is taken from a patient at a frequency of at least once a month and the level of at least one S67314 variant is measured using the obtained blood as a sample, for example. The level of a plurality of variants may be measured, and/or the level of the known FABP may optionally be measured, so that this information may be combined for a diagnosis. Depending upon the level measured (and/or levels measured) it may be determined that the administration of the agent is to be stopped and a quick protective measure of cardiac muscle needs to be taken, or alternatively that administration of adriamycin can be continued.

As described above with regard to the Table of Utilities, FABP is known as a marker for transmissible spongiform encephalopathies (TSEs), especially CJD, also as described with regard to US Patent Application No. 20030157580, hereby incorporated by reference as if fully set forth herein. The S67314 variants according to the present invention may also optionally be used for diagnosing a subject having a TSE, preferably CJD. Optionally, such a diagnostic test may be combined with a test suitable for detecting acute myocardial infarction (AMI) as described herein, in order to confirm diagnosis with TSE as opposed to AMI.

Also as described above with regard to the Table of Utilities, FABP is known as a marker for stroke, optionally including early diagnosis of stroke, also as described with regard to US Patent Application No. 20030100038, hereby incorporated by reference as if fully set forth herein. The S67314 variants according to the present invention may also optionally be used for diagnosing a subject having had a stroke. Optionally, such a diagnostic test may be combined with a test suitable for detecting acute myocardial infarction (AMI) as described herein, in order to confirm diagnosis with TSE as opposed to AMI.

Cluster S67314 belongs to a family of proteins which are known to have functions related to the cardiovascular system and functions, including but not limited to, RBP1, FABP7, FABP4, RBP5, PMP2, RBP7, CRABP1, FABP5, RBP2, CRABP2, FABP2, FABP6, FABP1. These functions are described below; one or more variants of cluster S67314 may optionally have one or more diagnostic utilities related to these functions. FABP in cardiac injury surpass the performance of the standard early marker myoglobin. The liver only contains liver-type FABP (L-FABP), but co-expression of H-FABP and L-FABP occurs in the kidney. Similarly, intestinal-type FABP (I-FABP) and L-FABP are found in intestines, and brain-type FABP (B-FABP) and H-FABP occur in the brain. Preliminary but promising applications of these proteins have been demonstrated for liver rejection, viability selection of kidneys from non-heart-beating donors (NHBD), inflammatory and ischemic bowel disease, traumatic brain injury and in the prevention of muscle injury in trained athletes (Pelsers M M, Hermens W T, Glatz J F: “Fatty acid-binding proteins as plasma markers of tissue injury”. Clin Chim Acta. 2005 February; 352(1-2):15-35.).

Some family members have functions related to fatty acid uptake, oxidation and overall metabolic homeostasis (reviewed in Hertzel A V, Bernlohr D A.: The mammalian fatty acid-binding protein multigene family: molecular and genetic insights into function. Trends Endocrinol Metab. 2000 July; 11(5):175-80). The genetic factors of obesity requires consideration of the genetic basis of the underlying etiological factors including energy expenditure and substrate utilization. Basic proteins involved in energy expenditure (the sodium-potassium ATPase and the uncoupling protein) or substrate utilization (fatty acid binding protein) (Goran M I: Genetic influences on human energy expenditure and substrate utilization. Behav Genet. 1997 July; 27(4):389-99.). Members of the family of fatty acid binding proteins are able to regulate mammary gland differentiation locally, and fatty acid binding is not required for this activity (Kurtz A, Spitzer E, Zschiesche W, Wellstein A, Grosse R.: Local control of mammary gland differentiation: mammary-derived growth inhibitor and pleiotrophin. Biochem Soc Symp. 1998; 63:51-69.). Lack of such differentiation may for example lead to a disease state such as cancer for example.

With regard to other cardiac functions, it should be noted that cytoplasmic fatty acid-binding proteins (FABPs) are a family of proteins, expressed in a tissue-specific manner, that bind fatty acid ligands and are involved in shuttling fatty acids to cellular compartments, modulating intracellular lipid metabolism, and regulating gene expression. Several members of the FABP family have been shown to have important roles in regulating metabolism and have links to the development of insulin resistance and the metabolic syndrome. Recent studies demonstrate a role for intestinal FABP in the control of dietary fatty acid absorption and chylomicron secretion. Heart FABP is essential for normal myocardial fatty acid oxidation and modulates fatty acid uptake in skeletal muscle. Liver FABP is directly involved in fatty acid ligand signaling to the nucleus and interacts with peroxisome proliferator-activated receptors in hepatocytes. The adipocyte FABP (aP2) has been shown to affect insulin sensitivity, lipid metabolism and lipolysis, and has recently been shown to play an important role in atherosclerosis. Interestingly, expression of aP2 by the macrophage promotes atherogenesis, thus providing a link between insulin resistance, intracellular fatty acid disposition, and foam cell formation. The FABPs are promising targets for the treatment of dyslipidemia, insulin resistance, and atherosclerosis in humans (Cytoplasmic fatty acid-binding proteins: emerging roles in metabolism and atherosclerosis; Current Opinion in Lipidology. 13(2):141-147, April 2002; Boord, Jeffrey B. a; Fazio, Sergio a,b; Linton, MacRae F. a,c.). All of these functions may optionally be diagnostic utilities of one or more S67314 variants according to the present invention.

The gene PS1D is antisense tail to tail and may therefore be co-regulated with one or more S67314 variants according to the present invention, and hence may have one or more utilities of S67314 variants according to the present invention as described herein.

Table 37 below describes diagnostic utilities for the cluster S67314 that were found through microarrays, including the statistical significance thereof and a reference. One or more S67314 variants according to the present invention may optionally have one or more of these utilities.

TABLE 37

| | Statistical | |
|---|---|---|
| Diagnostic utility | significance | reference |
| Gene over expressed in Diffuse Large B- | 0.045 to 3.6E−5 | 1. Alizadeh AA, Staudt LM Nature (2000) |
| Cell Lymphoma (vs Benign Lymphoid, | | Distinct types of diffuse large B-cell |
| CLL and Follicular Lymphoma). | | lymphoma identified by gene expression |
| | | profiling |
| Gene over expressed in metastasis prostate | 0.012 | 1. Dhanasekaran SM, Chinnaiyan AM |
| cancer (vs. primary) | | Nature (2001) Delineation of prognostic |
| | | biomarkers in prostate cancer |
| Gene over expressed in T1, T2+ bladder | 0.049 | 1. Dyrskjot L, Orntoft TF Nat Genet |
| cancer (vs. Ta) | | (2003) Identifying distinct classes of |
| | | bladder carcinoma using microarrays |
| Gene over expressed in Squamous Cell | 5.2E−6 | 1. Garber ME, Petersen I PNAS (2001) |
| Lung Carcinoma (vs. normal lung) | | Diversity of gene expression in |
| | | adenocarcinoma of the lung. |
| Gene over expressed in severe emphysema | | GNF database |
| (vs. normal or mild emphysema lung) | | (http://www.ncbi.nlm.nih.gov/projects/geo/): |
| | | GDS737, probe ID: 214285_at. |

Other non-limiting exemplary utilities for S67314 variants according to the present invention are described in greater detail below and also with regard to the previous section on clinical utility.

The heart-selective diagnostic marker prediction engine provided the following results with regard to cluster S67314. Predictions were made for selective expression of transcripts of this contig in heart tissue, according to the previously described methods. The numbers on the y-axis of the first figure below refer to weighted expression of ESTs in each category, as “parts per million” (ratio of the expression of ESTs for a particular cluster to the expression of all ESTs in that category, according to parts per million).

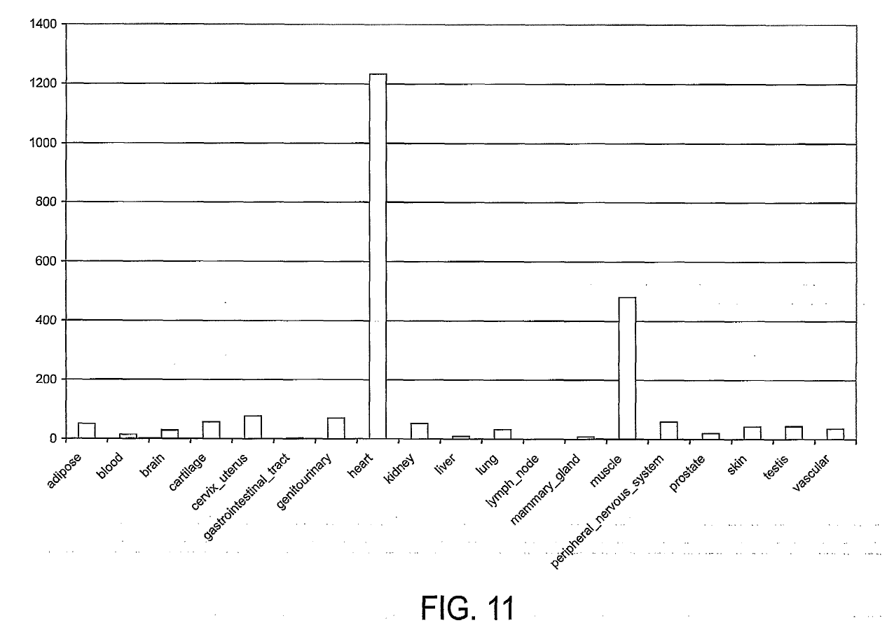

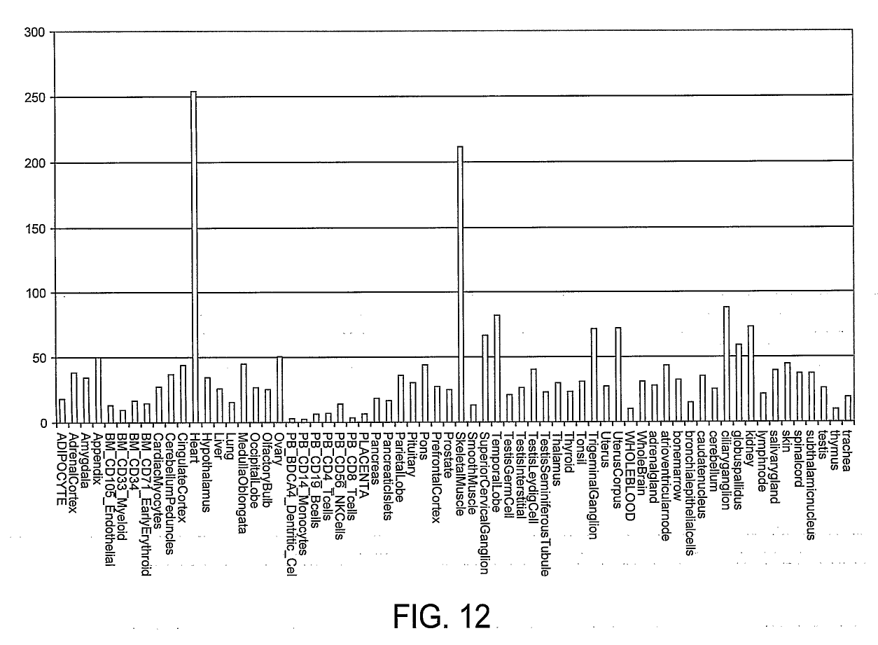

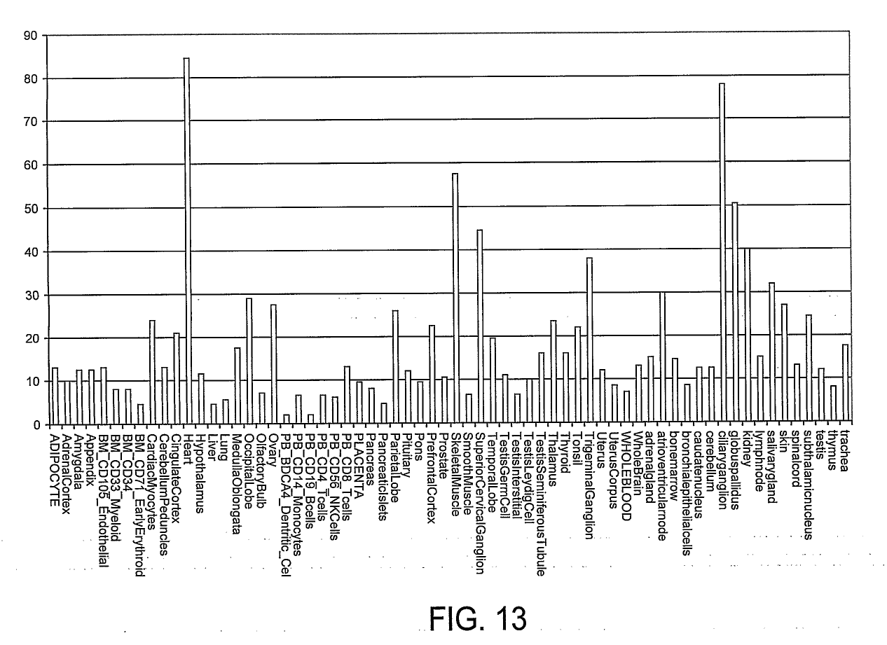

Overall, the following results were obtained as shown with regard to the histogram in FIG. 11, concerning the number of heart-specific clones in libraries/sequences; as well as with regard to the histogram in FIGS. 12-13 concerning the actual expression of oligonucleotides in various tissues, including heart.

This cluster was found to be selectively expressed in heart for the following reasons: in a comparison of the ratio of expression of the cluster in heart specific ESTs to the overall expression of the cluster in non-heart ESTs, which was found to be 13.6; the ratio of expression of the cluster in heart specific ESTs to the overall expression of the cluster in muscle-specific ESTs which was found to be 2.6; and fisher exact test P-values were computed both for library and weighted clone counts to check that the counts are statistically significant, and were found to be 2.30E-25.

One particularly important measure of specificity of expression of a cluster in heart tissue is the previously described comparison of the ratio of expression of the cluster in heart as opposed to muscle. This cluster was found to be specifically expressed in heart as opposed to non-heart ESTs as described above. However, many proteins have been shown to be generally expressed at a higher level in both heart and muscle, which is less desirable. For this cluster, as described above, the ratio of expression of the cluster in heart specific ESTs to the overall expression of the cluster in muscle-specific ESTs which was found to be 13.6, which clearly supports specific expression in heart tissue.

As noted above, cluster S67314 features 4 transcript(s), which were listed in Table 32 above. These transcript(s) encode for protein(s) which are variant(s) of protein Fatty acid-binding protein (SEQ ID NO:112), heart. A description of each variant protein according to the present invention is now provided.

Variant protein S67314_PEA_{—}1_P4 (SEQ ID NO:114) according to the present invention has an amino acid sequence; it is encoded by transcript(s) S67314_PEA_{—}1_T4 (SEQ ID NO:100). An alignment is given to the known protein (Fatty acid-binding protein (SEQ ID NO:112), heart). One or more alignments to one or more previously published protein sequences are given in the alignment table located on the attached CDROM. A brief description of the relationship of the variant protein according to the present invention to each such aligned protein is as follows:

Comparison report between S67314_PEA_{—}1_P4 (SEQ ID NO:114) and FABH_HUMAN_V1 (SEQ ID NO:113):

1. An isolated chimeric polypeptide encoding for S67314_PEA_{—}1_P4 (SEQ ID NO:114), comprising a first amino acid sequence being at least 90% homologous to MVDAFLGTWKLVDSKNFDDYMKSLGVGFATRQVASMTKPTTIIEKNGDILTLKTHSTFKNTEISFK LGVEFDETTADDRKVKSIVTLDGGKLVHLQKWDGQETTLVRELIDGKLIL corresponding to amino acids 1-116 of FABH_HUMAN_V1 (SEQ ID NO:113), which also corresponds to amino acids 1-116 of S67314_PEA_{—}1_P4 (SEQ ID NO:114), and a second amino acid sequence being at least 70%, optionally at least 80%, preferably at least 85%, more preferably at least 90% and most preferably at least 95% homologous to a polypeptide having the sequence VRWATLELYLIGYYYCSFSQACSKKPSPPLRAVEAGTREWLWVRVVSGGNFLCSGFGLTQAGTQI LPYRLHDCGQITFSKCNCKTGINNTNLVGLLGSL (SEQ ID NO: 635) corresponding to amino acids 117-215 of S67314_PEA_{—}1_P4 (SEQ ID NO:114), wherein said first amino acid sequence and second amino acid sequence are contiguous and in a sequential order.

2. An isolated polypeptide encoding for a tail of S67314_PEA_{—}1_P4 (SEQ ID NO:114), comprising a polypeptide being at least 70%, optionally at least about 80%, preferably at least about 85%, more preferably at least about 90% and most preferably at least about 95% homologous to the sequence VRWATLELYLIGYYYCSFSQACSKKPSPPLRAVEAGTREWLWVRVVSGGNFLCSGFGLTQAGTQI LPYRLHDCGQITFSKCNCKTGINNTNLVGLLGSL (SEQ ID NO: 635) in S67314_PEA_{—}1_P4 (SEQ ID NO:114).

It should be noted that the known protein sequence (FABH_HUMAN (SEQ ID NO:112)) has one or more changes than the sequence named as being the amino acid sequence for FABH_HUMAN_Vi (SEQ ID NO:113). These changes were previously known to occur and are listed in table 38 below.

TABLE 38
Changes to FABH_HUMAN_V1 (SEQ ID NO: 113)

| SNP position(s) on amino | |
|---|---|
| acid sequence | Type of change |
| 1 | init_met |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

The variant protein has the following domains, as determined by using InterPro. The domains are described in Table 39:

TABLE 39
InterPro domain(s)

| | | | Position(s) on |
|---|---|---|---|
| InterPro ID | Domain description | Analysis type | protein |
| IPR000463 | Cytosolic fatty-acid | FPrintScan | 5-27, 64-80 |
| | binding protein | | |
| IPR000566 | Lipocalin-related | HMMPfam | 4-117 |
| | protein | | |
| | and Bos/Can/Equ | | |
| | allergen | | |
| IPR000463 | Cytosolic fatty-acid | ScanRegExp | 7-24 |
| | binding | | |
| | protein | | |

Variant protein S67314_PEA_{—}1_P4 (SEQ ID NO:114) is encoded by the following transcript(s): S67314_PEA_{—}1_T4 (SEQ ID NO:100). The coding portion of transcript S67314_PEA_{—}1_T4 (SEQ ID NO:100) starts at position 925 and ends at position 1569.

Variant protein S67314_PEA_{—}1_P5 (SEQ ID NO:115) according to the present invention has an amino acid sequence; it is encoded by transcript(s) S67314_PEA_{—}1_T5 (SEQ ID NO:101). An alignment is given to the known protein (Fatty acid-binding protein (SEQ ID NO:112), heart). One or more alignments to one or more previously published protein sequences are given in the alignment table located on the attached CDROM. A brief description of the relationship of the variant protein according to the present invention to each such aligned protein is as follows:

Comparison report between S67314_PEA_{—}1_P5 (SEQ ID NO:115) and FABH_HUMAN_V1 (SEQ ID NO:113):

1. An isolated chimeric polypeptide encoding for S67314_PEA_{—}1_P5 (SEQ ID NO:115), comprising a first amino acid sequence being at least 90% homologous to MVDAFLGTWKLVDSKNFDDYMKSLGVGFATRQVASMTKPTTIIEKNGDILTLKTHSTFKNTEISFK LGVEFDETTADDRKVKSIVTLDGGKLVHLQKWDGQETTLVRELIDGKLIL corresponding to amino acids 1-116 of FABH_HUMAN_V1 (SEQ ID NO:113), which also corresponds to amino acids 1-116 of S67314_PEA_{—}1_P5 (SEQ ID NO:115), and a second amino acid sequence being at least 70%, optionally at least 80%, preferably at least 85%, more preferably at least 90% and most preferably at least 95% homologous to a polypeptide having the sequence DVLTAWPSIYRRQVKVLREDEITILPWHLQWSREKATKLLRPTLPSYNNHGWEELRVGKSIV (SEQ ID NO: 636) corresponding to amino acids 117-178 of S67314_PEA_{—}1_P5 (SEQ ID NO:115), wherein said first amino acid sequence and second amino acid sequence are contiguous and in a sequential order.

2. An isolated polypeptide encoding for a tail of S67314_PEA_{—}1_P5 (SEQ ID NO:115), comprising a polypeptide being at least 70%, optionally at least about 80%, preferably at least about 85%, more preferably at least about 90% and most preferably at least about 95% homologous to the sequence DVLTAWPSIYRRQVKVLREDEITILPWHLQWSREKATKLLRPTLPSYNNHGWEELRVGKSIV (SEQ ID NO: 636) in S67314_PEA_{—}1_P5 (SEQ ID NO:115).

It should be noted that the known protein sequence (FABH_HUMAN (SEQ ID NO:112)) has one or more changes than the sequence named as being the amino acid sequence for FABH_HUMAN_V1 (SEQ ID NO:113). These changes were previously known to occur and are listed in table 40 below.

TABLE 40
Changes to FABH_HUMAN_V1 (SEQ ID NO:113)

| SNP position(s) on amino | |
|---|---|
| acid sequence | Type of change |
| 1 | init_met |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

The variant protein has the following domains, as determined by using InterPro. The domains are described in Table 41:

TABLE 41
InterPro domain(s)

| | | | Position(s) on |
|---|---|---|---|
| InterPro ID | Domain description | Analysis type | protein |
| IPR000463 | Cytosolic fatty-acid | FPrintScan | 5-27, 64-80 |
| | binding protein | | |
| IPR000566 | Lipocalin-related | HMMPfam | 4-115 |
| | protein | | |
| | and Bos/Can/Equ | | |
| | allergen | | |
| IPR000463 | Cytosolic fatty-acid | ScanRegExp | 7-24 |
| | binding | | |
| | protein | | |

Variant protein S67314_PEA_{—}1_P5 (SEQ ID NO:115) is encoded by the following transcript(s): S67314_PEA_{—}1_T5 (SEQ ID NO:101). The coding portion of transcript S67314_PEA_{—}1_T5 (SEQ ID NO:101) starts at position 925 and ends at position 1458.

Variant protein S67314_PEA_{—}1_P6 (SEQ ID NO:116) according to the present invention has an amino acid sequence; it is encoded by transcript(s) S67314_PEA_{—}1_T6 (SEQ ID NO:102). An alignment is given to the known protein (Fatty acid-binding protein (SEQ ID NO:112), heart) at the end of the application. One or more alignments to one or more previously published protein sequences are given in the alignment table located on the attached CDROM. A brief description of the relationship of the variant protein according to the present invention to each such aligned protein is as follows:

Comparison report between S67314_PEA_{—}1_P6 (SEQ ID NO:116) and FABH_HUMAN_V1 (SEQ ID NO:113):

1. An isolated chimeric polypeptide encoding for S67314_PEA_{—}1_P6 (SEQ ID NO:116), comprising a first amino acid sequence being at least 90% homologous to MVDAFLGTWKLVDSKNFDDYMKSLGVGFATRQVASMTKPTTIIEKNGDILTLKTHSTFKNTEISFK LGVEFDETTADDRKVKSIVTLDGGKLVHLQKWDGQETTLVRELIDGKLIL corresponding to amino acids 1-116 of FABH_HUMAN_V1 (SEQ ID NO:113), which also corresponds to amino acids 1-116 of S67314_PEA_{—}1_P6 (SEQ ID NO:116), and a second amino acid sequence being at least 70%, optionally at least 80%, preferably at least 85%, more preferably at least 90% and most preferably at least 95% homologous to a polypeptide having the sequence MEKLQLRNVK (SEQ ID NO: 637) corresponding to amino acids 117-126 of S67314_PEA_{—}1_P6 (SEQ ID NO:116), wherein said first amino acid sequence and second amino acid sequence are contiguous and in a sequential order.

2. An isolated polypeptide encoding for a tail of S67314_PEA_{—}1_P6 (SEQ ID NO:116), comprising a polypeptide being at least 70%, optionally at least about 80%, preferably at least about 85%, more preferably at least about 90% and most preferably at least about 95% homologous to the sequence MEKLQLRNVK (SEQ ID NO: 637) in S67314_PEA_{—}1_P6 (SEQ ID NO:116).

It should be noted that the known protein sequence (FABH_HUMAN (SEQ ID NO:112)) has one or more changes than the sequence named as being the amino acid sequence for FABH_HUMAN_V1 (SEQ ID NO:113). These changes were previously known to occur and are listed in table 42 below.

TABLE 42
Changes to FABH_HUMAN_V1 (SEQ ID NO:113)

| SNP position(s) on amino | |
|---|---|
| acid sequence | Type of change |
| 1 | init_met |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

The variant protein has the following domains, as determined by using InterPro. The domains are described in Table 43:

TABLE 43
InterPro domain(s)

| | | | Position(s) on |
|---|---|---|---|
| InterPro ID | Domain description | Analysis type | protein |
| IPR000463 | Cytosolic fatty-acid | FPrintScan | 5-27, 64-80 |
| | binding protein | | |
| IPR000566 | Lipocalin-related | HMMPfam | 4-119 |
| | protein | | |
| | and Bos/Can/Equ | | |
| | allergen | | |
| IPR000463 | Cytosolic fatty-acid | ScanRegExp | 7-24 |
| | binding | | |
| | protein | | |

Variant protein S67314_PEA_{—}1_P6 (SEQ ID NO:116) is encoded by the following transcript(s): S67314_PEA_{—}1_T6 (SEQ ID NO:102). The coding portion of transcript S67314_PEA_{—}1_T6 (SEQ ID NO:102) starts at position 925 and ends at position 1302.

Variant protein S67314_PEA_{—}1_P7 (SEQ ID NO:117) according to the present invention has an amino acid sequence; it is encoded by transcript(s) S67314_PEA_{—}1_T7 (SEQ ID NO:103). An alignment is given to the known protein (Fatty acid-binding protein (SEQ ID NO:112), heart) at the end of the application. One or more alignments to one or more previously published protein sequences are given in the alignment table located on the attached CDROM. A brief description of the relationship of the variant protein according to the present invention to each such aligned protein is as follows:

Comparison report between S67314_PEA_{—}1_P7 (SEQ ID NO:117) and FABH_HUMAN_V1 (SEQ ID NO:113):

1. An isolated chimeric polypeptide encoding for S67314_PEA_{—}1_P7 (SEQ ID NO:117), comprising a first amino acid sequence being at least 90% homologous to MVDAFLGTWKLVDSKNFDDYMKSL corresponding to amino acids 1-24 of FABH_HUMAN_V1 (SEQ ID NO:113), which also corresponds to amino acids 1-24 of S67314_PEA_{—}1_P7 (SEQ ID NO:117), a second amino acid sequence being at least 70%, optionally at least 80%, preferably at least 85%, more preferably at least 90% and most preferably at least 95% homologous to a polypeptide having the sequence AHILITFPLPS (SEQ ID NO: 638) corresponding to amino acids 25-35 of S67314_PEA_{—}1_P7 (SEQ ID NO:117), and a third amino acid sequence being at least 90% homologous to GVGFATRQVASMTKPTTIIEKNGDILTLKTHSTFKNTEISFKLGVEFDETTADDRKVKSIVTLDGGK LVHLQKWDGQETTLVRELIDGKLILTLTHGTAVCTRTYEKEA corresponding to amino acids 25-133 of FABH_HUMAN_V1 (SEQ ID NO:113), which also corresponds to amino acids 36-144 of S67314_PEA_{—}1_P7 (SEQ ID NO:117), wherein said first amino acid sequence, second amino acid sequence and third amino acid sequence are contiguous and in a sequential order.

2. An isolated polypeptide encoding for an edge portion of S67314_PEA_{—}1_P7 (SEQ ID NO:117), comprising an amino acid sequence being at least 70%, optionally at least about 80%, preferably at least about 85%, more preferably at least about 90% and most preferably at least about 95% homologous to the sequence encoding for AHILITFPLPS (SEQ ID NO: 638), corresponding to S67314_PEA_{—}1_P7 (SEQ ID NO:117).

It should be noted that the known protein sequence (FABH_HUMAN (SEQ ID NO:112)) has one or more changes than the sequence named as being the amino acid sequence for FABH_HUMAN_V1 (SEQ ID NO:113). These changes were previously known to occur and are listed in table 44 below.

TABLE 44
Changes to FABH_HUMAN_V1 (SEQ ID NO:113)

| SNP position(s) on amino | |
|---|---|
| acid sequence | Type of change |
| 1 | init_met |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

The variant protein has the following domains, as determined by using InterPro. The domains are described in Table 45:

TABLE 45
InterPro domain(s)

| | | | Position(s) on |
|---|---|---|---|
| InterPro ID | Domain description | Analysis type | protein |
| IPR000463 | Cytosolic fatty-acid | FPrintScan | 122-142, 5-27 |
| | binding | | |
| | protein | | |
| IPR000566 | Lipocalin-related | HMMPfam | 4-143 |
| | protein | | |
| | and Bos/Can/Equ | | |
| | allergen | | |
| IPR000463 | Cytosolic fatty-acid | ScanRegExp | 7-24 |
| | binding | | |
| | protein | | |

Variant protein S67314_PEA_{—}1_P7 (SEQ ID NO:117) is encoded by the following transcript(s): S67314_PEA_{—}1_T7 (SEQ ID NO:103). The coding portion of transcript S67314_PEA_{—}1_T7 (SEQ ID NO:103) starts at position 925 and ends at position 1356.

Table 46 below describes the starting and ending position of S67314_PEA_{—}1_node_{—}4 (SEQ ID NO:105) on each of the relevant transcripts. Experimental results of this segment are described below.

TABLE 46
Segment location on transcripts

| | Segment | Segment |
|---|---|---|
| | starting | ending |
| Transcript name | position | position |
| S67314_PEA_1_T4 (SEQ ID NO:100) | 998 | 1170 |
| S67314_PEA_1_T5 (SEQ ID NO:101) | 998 | 1170 |
| S67314_PEA_1_T6 (SEQ ID NO:102) | 998 | 1170 |
| S67314_PEA_1_T7 (SEQ ID NO:103) | 1031 | 1203 |

Table 47 below describes the starting and ending position of S67314_PEA_{—}1_node_{—}11 (SEQ ID NO:106) on each of the relevant transcripts. Experimental results for this segment are described below.

TABLE 47
Segment location on transcripts

| | Segment | Segment |
|---|---|---|
| | starting | ending |
| Transcript name | position | position |
| S67314_PEA_1_T4 (SEQ ID NO:100) | 1273 | 2110 |

Table 48 below describes the starting and ending position of S67314_PEA_{—}1_node_{—}15 (SEQ ID NO:108) on each of the relevant transcripts. Experimental results for this segment are described below.

TABLE 48
Segment location on transcripts

| | Segment | Segment |
|---|---|---|
| | starting | ending |
| Transcript name | position | position |
| S67314_PEA_1_T5 (SEQ ID NO:101) | 1273 | 1733 |

Expression of FABH_HUMAN (SEQ ID NO:112) Fatty acid-binding protein (SEQ ID NO:112) transcripts which are detectable by amplicon as depicted in sequence name S67314seg11 (SEQ ID NO: 120) specifically in heart tissue:

Expression of FABH_HUMAN (SEQ ID NO:112) Fatty acid-binding protein (SEQ ID NO:112) transcripts detectable by or according to seg11, S67314 seg11 (SEQ ID NO:120) amplicon and S67314 seg11F (SEQ ID NO:118) and S67314 seg11R (SEQ ID NO:119) primers was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicon was normalized to the geometric mean of the quantities of the housekeeping genes. The normalized quantity of each RT sample was then divided by the median of the quantities of the heart samples (Sample Nos. 44, 45, 46, Table 7, above, “Tissue samples in normal panel”), to obtain a value of expression for each sample relative to median of the heart tissue.

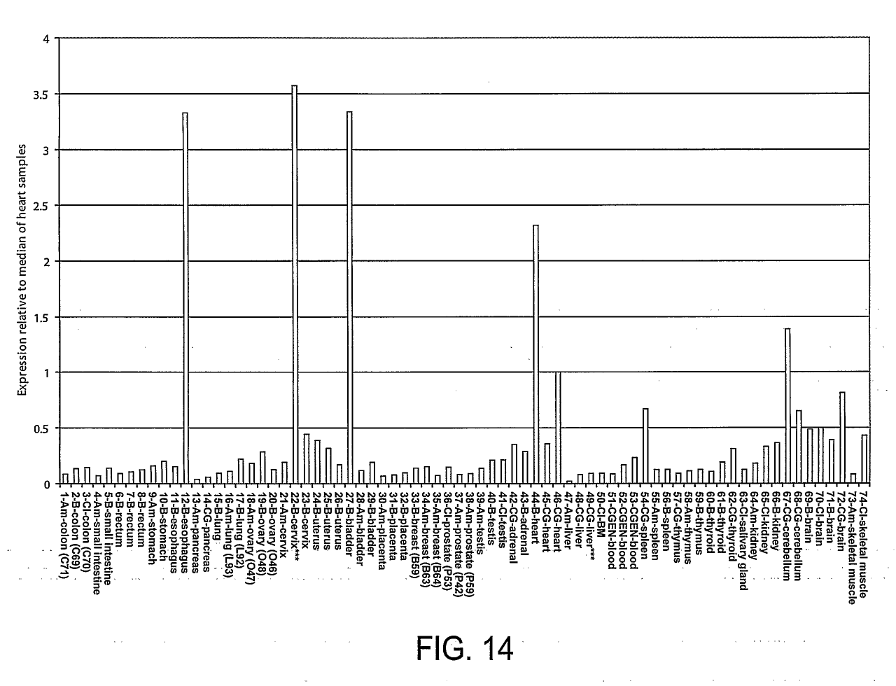

FIG. 14 is a histogram showing specific expression of the above-indicated FABH_HUMAN (SEQ ID NO:112) Fatty acid-binding protein (SEQ ID NO:112) transcripts in heart tissue samples as opposed to other tissues.

As is evident from FIG. 14, the expression of FABH_HUMAN (SEQ ID NO:112) Fatty acid-binding protein (SEQ ID NO:112) transcripts detectable by the above amplicon in normal heart tissue samples was significantly higher than in the other samples (Sample Nos. 1-43, 47-74, Table 7, “Tissue samples in normal panel”).

Primer pairs are also optionally and preferably encompassed within the present invention; for example, for the above experiment, the following primer pair was used as a non-limiting illustrative example only of a suitable primer pair: S67314 seg11F (SEQ ID NO:118) forward primer; and S67314 seg11R (SEQ ID NO:119) reverse primer.

The present invention also preferably encompasses any amplicon obtained through the use of any suitable primer pair; for example, for the above experiment, the following amplicon was obtained as a non-limiting illustrative example only of a suitable amplicon: S67314 seg11 (SEQ ID NO:120).

S67314 seg11F (SEQ ID NO:118):
TCCCCTGAGAGCTGTAGAAGCT
S67314 seg11R (SEQ ID NO:119):
CGGCCTGTGTGAGTCCAAA
S67314 seg11 (SEQ ID NO:120):
TCCCCTGAGAGCTGTAGAAGCTGGGACAAGAGAGTGGTTGTGGGTCAGGG
TGGTATCAGGTGGGAATTTTCTGTGTAGTGGCTTTGGACTCACACAGGCC
G

Expression of FABH_HUMAN (SEQ ID NO:112) Fatty acid-binding protein (SEQ ID NO:112) S67314 transcripts, which are detectable by amplicon as depicted in sequence name S67314 seg15 (SEQ ID NO:123) specifically in heart tissue:

Expression of FABH_HUMAN (SEQ ID NO:112) Fatty acid-binding protein (SEQ ID NO:112) transcripts detectable by or according to segment 15, S67314 seg15 (SEQ ID NO:123) amplicon and S67314 seg15F (SEQ ID NO: 121) and S67314 seg15R (SEQ ID NO: 122) primers was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicon was normalized to the geometric mean of the quantities of the housekeeping genes. The normalized quantity of each RT sample was then divided by the median of the quantities of the heart samples (Sample Nos. 44-46, Table 7, above, “Tissue samples in normal panel”), to obtain a value of expression for each sample relative to median of the heart tissue.

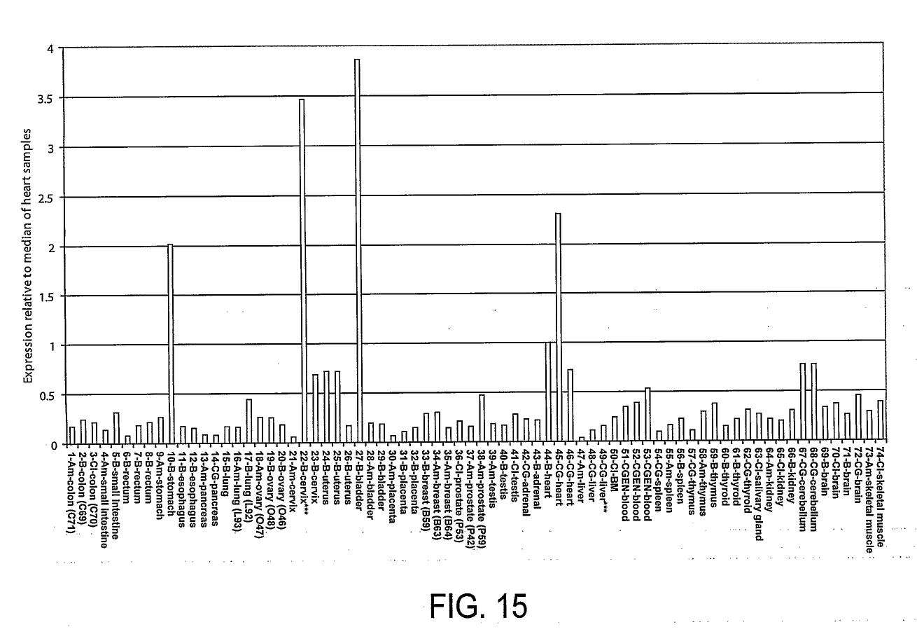

FIG. 15 is a histogram showing specific expression of the above-indicated FABH_HUMAN (SEQ ID NO:112) Fatty acid-binding protein (SEQ ID NO:112) transcripts in heart tissue samples as opposed to other tissues.

As is evident from FIG. 15, the expression of FABH_HUMAN (SEQ ID NO:112) Fatty acid-binding protein (SEQ ID NO:112) transcripts detectable by the above amplicon in normal heart tissue samples was significantly higher than in most other samples (Sample Nos. 1-9, 11-21, 23-26, 28-43, 47-74 Table 7, “Tissue samples in normal panel”).

Primer pairs are also optionally and preferably encompassed within the present invention; for example, for the above experiment, the following primer pair was used as a non-limiting illustrative example only of a suitable primer pair: S67314 seg15F (SEQ ID NO: 121) forward primer; and S67314 seg15R (SEQ ID NO: 122) reverse primer.

The present invention also preferably encompasses any amplicon obtained through the use of any suitable primer pair; for example, for the above experiment, the following amplicon was obtained as a non-limiting illustrative example only of a suitable amplicon: S67314 seg15 (SEQ ID NO:123).

Forward primer - S67314 seg15-F (SEQ ID NO:121):
TTCCTTGGCATCTCCAATGG
Reverse primer - S67314 seg15-R (SEQ ID NO:122):
GCCAACTCTCAGCTCCTCCC
Amplicon (SEQ ID NO:123):
TTCCTTGGCATCTCCAATGGAGTAGAGAGAAGGCAACAAAGCTTCTCAGA
CCCACATTACCGAGCTATAACAACCATGGCTGGGAGGAGCTGAGAGTTGG
C

Expression of FABH_HUMAN (SEQ ID NO:112) Fatty acid-binding protein (SEQ ID NO:112) S67314 transcripts which are detectable by amplicon as depicted in sequence name S67314seg4 (SEQ ID NO: 126) specifically in heart tissue:

Expression of FABH_HUMAN (SEQ ID NO:112) Fatty acid-binding protein (SEQ ID NO:112) S67314 transcripts detectable by or according to seg4 node, S67314 seg4 (SEQ ID NO: 126) amplicon and primers S67314seg4F (SEQ ID NO: 124) and S67314seg4R (SEQ ID NO: 125) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44-46, Table 7, above, “Tissue samples in normal panel”), to obtain a value of relative expression for each sample relative to median of the heart samples.

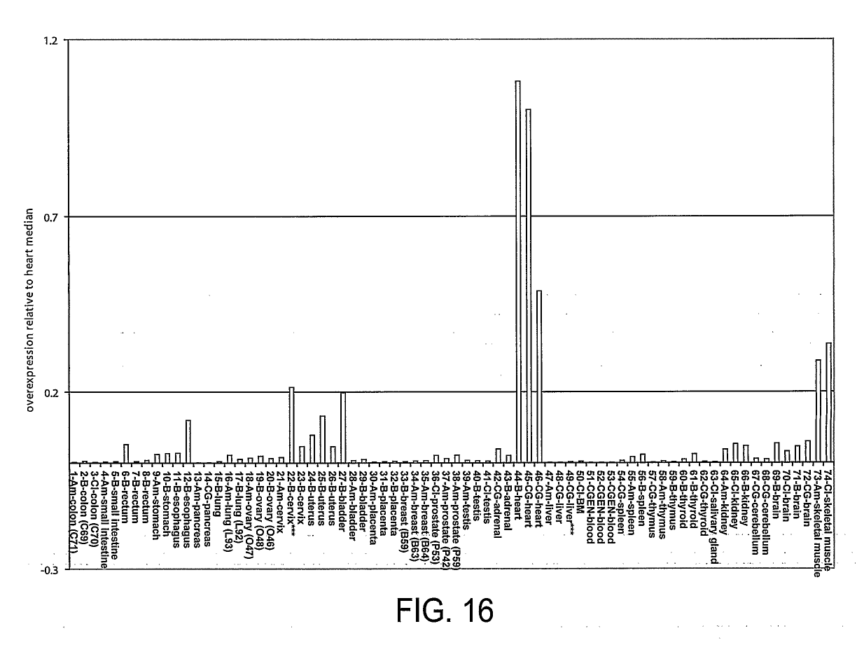

FIG. 16 is a histogram showing relative expression of the above-indicated FABH_HUMAN (SEQ ID NO:112) Fatty acid-binding protein (SEQ ID NO:112) transcripts in heart tissue samples as opposed to other tissues.

As is evident from FIG. 16, the expression of FABH_HUMAN (SEQ ID NO:112) Fatty acid-binding protein (SEQ ID NO:112) transcripts detectable by the above amplicon in normal heart tissue samples was significantly higher than in the other samples (Sample Nos. 44-46 Table 7, “Tissue samples in normal panel”).

Primer pairs are also optionally and preferably encompassed within the present invention; for example, for the above experiment, the following primer pair was used as a non-limiting illustrative example only of a suitable primer pair: S67314seg4F (SEQ ID NO: 124) forward primer; and S67314seg4R (SEQ ID NO: 125) reverse primer.

The present invention also preferably encompasses any amplicon obtained through the use of any suitable primer pair; for example, for the above experiment, the following amplicon was obtained as a non-limiting illustrative example only of a suitable amplicon: S67314seg4 (SEQ ID NO: 126).

Primers:

Forward primer S67314 seg4F (SEQ ID NO:124):
CCAAGCCTACCACAATCATCG
Reverse primer S67314 seg4R (SEQ ID NO:125):
CTCCACCCCAACTTAAAGCT
Amplicon S67314 seg4 (SEQ ID NO:126):
CCAAGCCTACCACAATCATCGAAAAGAATGGGGACATTCTCACCCTAAAA
ACACACAGCACCTTCAAGAACACAGAGATCAGCTTTAAGTTGGGGGTGGA
G

### Description for Cluster HUMNATPEP

Cluster HUMNATPEP features 4 transcript(s) and 7 segment(s) of interest, the names for which are given in Tables 49 and 50, respectively. The selected protein variants are given in table 51.

TABLE 49
Transcripts of interest

| Transcript Name | |
|---|---|
| HUMNATPEP_PEA_1_T1 | (SEQ ID NO:127) |
| HUMNATPEP_PEA_1_T2 | (SEQ ID NO:128) |
| HUMNATPEP_PEA_1_T3 | (SEQ ID NO:129) |
| HUMNATPEP_PEA_1_T4 | (SEQ ID NO:130) |

TABLE 50
Segments of interest

Segment Name

| | |
|---|---|
| HUMNATPEP_PEA_1_node 0 (SEQ ID NO:131) | HUMNATPEP_PEA_1_T1 (SEQ ID |
| | NO:127), HUMNATPEP_PEA_1_T2 (SEQ |
| | ID NO:128), HUMNATPEP_PEA_1_T3 |
| | (SEQ ID NO:129) and |
| | HUMNATPEP_PEA_1_T4 (SEQ ID |
| | NO:130) |
| HUMNATPEP_PEA_1_node 1 (SEQ ID NO:132) | HUMNATPEP_PEA_1_T1 (SEQ ID |
| | NO:127), HUMNATPEP_PEA_1_T2 (SEQ |
| | ID NO:128) and HUMNATPEP_PEA_1_T3 |
| | (SEQ ID NO:129) |
| HUMNATPEP_PEA_1_node 2 (SEQ ID NO:133) | HUMNATPEP_PEA_1_T2 (SEQ ID |
| | NO:128) and HUMNATPEP_PEA_1_T3 |
| | (SEQ ID NO:129) |
| HUMNATPEP_PEA_1_node 3 (SEQ ID NO:134) | HUMNATPEP_PEA_1_T1 (SEQ ID |
| | NO:127), HUMNATPEP_PEA_1_T2 (SEQ |
| | ID NO:128) and HUMNATPEP_PEA_1_T3 |
| | (SEQ ID NO:129) |
| HUMNATPEP_PEA_1_node 4 (SEQ ID NO:135) | HUMNATPEP_PEA_1_T1 (SEQ ID |
| | NO:127), HUMNATPEP_PEA_1_T2 (SEQ |
| | ID NO:128), HUMNATPEP_PEA_1_T3 |
| | (SEQ ID NO:129) and |
| | HUMNATPEP_PEA_1_T4 (SEQ ID |
| | NO:130) |
| HUMNATPEP_PEA_1_node 5 (SEQ ID NO:136) | HUMNATPEP_PEA_1_T1 (SEQ ID |
| | NO:127) and HUMNATPEP_PEA_1_T3 |
| | (SEQ ID NO:129) |
| HUMNATPEP_PEA_1_node 6 (SEQ ID NO:137) | HUMNATPEP_PEA_1_T1 (SEQ ID |
| | NO:127), HUMNATPEP_PEA_1_T2 (SEQ |
| | ID NO:128), HUMNATPEP_PEA_1_T3 |
| | (SEQ ID NO:129) and |
| | HUMNATPEP_PEA_1_T4 (SEQ ID |
| | NO:130) |

TABLE 51
Proteins of interest

| Protein Name | Corresponding Transcript(s) |
|---|---|
| HUMNATPEP_PEA_1_P2 (SEQ ID NO:139) | HUMNATPEP_PEA_1_T1 (SEQ ID NO:127) |
| HUMNATPEP_PEA_1_P3 (SEQ ID NO:140) | HUMNATPEP_PEA_1_T2 (SEQ ID NO:128) |
| | HUMNATPEP_PEA_1_T3 (SEQ ID NO:129) |
| HUMNATPEP_PEA_1_P7 (SEQ ID NO:141) | HUMNATPEP_PEA_1_T4 (SEQ ID NO:130) |

These sequences are variants of the known protein Natriuretic peptides B precursor [Contains: Gamma-brain natriuretic peptide; Brain natriuretic peptide 32 (BNP-32)] (SwissProt accession identifier ANFB_HUMAN (SEQ ID NO:138)), referred to herein as the previously known protein.

Protein Natriuretic peptides B precursor (SEQ ID NO: 138) [Contains: Gamma-brain natriuretic peptide; Brain natriuretic peptide 32 (BNP-32)] is known or believed to have the following function(s): Acts as a cardiac hormone with a variety of biological actions including natriuresis, diuresis, vasorelaxation, and inhibition of renin and aldosterone secretion. It is thought to play a key role in cardiovascular homeostasis. Helps restore the body's salt and water balance. Improves heart function. Known polymorphisms for this sequence are as shown in Table 52.

TABLE 52
Amino acid mutations for Known Protein

| SNP position(s) | |
|---|---|
| on amino | |
| acid sequence | Comment |
| 25 | R -> L (in dbSNP:5227)./FTId = VAR_014580. |
| 47 | R -> H (in dbSNP:5229)./FTId = VAR_014581. |
| 93 | M -> L (in dbSNP:5230)./FTId = VAR_014582. |

Protein Natriuretic peptides B precursor (SEQ ID NO: 138) [Contains: Gamma-brain natriuretic peptide; Brain natriuretic peptide 32 (BNP-32)] localization is believed to be Secreted.

The previously known protein also has the following indication(s) and/or potential therapeutic use(s): Hepatic dysfunction; Hypertension; Heart failure; Asthma; Renal failure. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Atrial peptide agonist; Diuretic. A therapeutic role for a protein represented by the cluster has been predicted. The cluster was assigned this field because there was information in the drug database or the public databases (e.g., described herein above) that this protein, or part thereof, is used or can be used for a potential therapeutic indication: Hepatoprotective; Antihypertensive; Antihypertensive, diuretic; Cardiostimulant; Vasodilator, coronary; Urological; Antiasthma; COPD treatment.

The following GO Annotation(s) apply to the previously known protein. The following annotation(s) were found: fluid secretion, which are annotation(s) related to Biological Process; diuretic hormone, which are annotation(s) related to Molecular Function; and extracellular space, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of HUMNATPEP) may optionally have one or more of the utilities described with regard to variants of ANP (cluster HUMCDDANF).

According to optional but preferred embodiments of the present invention, variants of this cluster according to the present invention may also have one or more of the following utilities, as described with regard to Table 53a below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 53a
Utilities for Variants of HUMNATPEP, related to BNP

| | | |
|---|---|---|
| Marker for congestive | BNP levels in serum increase in CHF, | Clin Cardiol. 2004 |
| heart failure (CHF) | NT-proBNP is elevated in patients with stable angina | Sep; 27(9): 489-94. |
| and for acute and | pectoris and has a close correlation to disease severity | Am Heart J. 2004 |
| chronic coronary heart | | Oct; 148(4): 612-20. |
| disease | | |
| marker of long-term | NT-pro-BNP was measured in baseline serum samples | N Engi J Med. 2005 |
| mortality in patients | from 1034 patients referred for angiography because of | Feb 17; 352(7): 666- |
| with stable coronary | symptoms or signs of coronary heart disease. The rate | 75. |
| disease | of death from all causes was determined after a median | |
| | follow-up of nine years. The median NT-pro-BNP | |
| | level was significantly lower among patients who | |
| | survived than among those who died | |
| Marker for lone atrial | Median levels of nt-pro-BNP were significantly | J Am Coil Cardiol. |
| fibrillation | elevated in subjects with lone atrial fibrillation as | 2005 Jan 4; 45(1): 82-. |
| | compared with control subjects | 6. |
| Marker for coronary | BNP levels in patients with unstable angina and | Clin Exp Med. 2004 |
| heart disease, | myocardial infarction were significantly increased with | Sep; 4(1): 44-9. |
| especially in acute | respect to the group with stable angina (P < 0.01). | |
| coronary syndromes, | Analysis of peptide levels in relation to the number of | |
| even in the absence of | involved vessels demonstrated a significant increase in | |
| systolic dysfunction | patients with three-vessel disease compared with | |
| | subjects with one or two vessels involved (P < 0.03); | |
| | among subjects with mono-vessel disease, patients with | |
| | left descendent anterior stenosis had a more-marked | |
| | BNP elevation than subjects with stenosis in other | |
| | regions (P < 0.01). | |
| Predicts (in the | The prognostic value of plasma N-terminal-pro-brain | Am J Cardiol. 2004 |
| medium-term) good | natriuretic peptide (NT-pro-BNP) in 71 transplanted | Dec 15; 94(12): 1585- |
| chances of survival in | heart recipients during a 38 +/− 2 month follow-up. The | 7. |
| transplanted heart | negative predictive value of NT-pro-BNP levels | |
| recipients | </=800 pg/ml to predict death was 97% (95% | |
| | confidence interval 92 to 100). | |
| BNP levels before | Among 1,172 consecutive patients, the occurrence of | Am J Cardiol. 2004 |
| percutaneous coronary | death or MI increased significantly with baseline NT- | Dec 15; 94(12): 1481- |
| intervention (PCI) | pro-BNP before PCI | 5. |
| provides important, | | |
| independent prognostic | | |
| information for the | | |
| occurrence of death or | | |
| nonfatal MI during | | |
| long-term follow-up. | | |
| BNP and ANP can be | Plasma BNP (pmol/L) and ANP (pmol/L) were | J Renin Angiotensin |
| useful diagnostic tools | determined in 68 hypertensive patients with dilated | Aldosterone Syst. |
| in hypertensive CHF | cardiomyopathy, NYHA class III-IV and ejection | 2004 Sep; 5(3): 121-9. |
| patients with | fraction (EF) < or = 40%, and in 26 normal controls. | |
| moderate-to-severe LV | Statistical analysis for BNP and ANP was done by | |
| dysfunction and also | Students t-test. The patient group was randomly | |
| used for progonosis | subdivided into two subgroups of 34 patients, each | |
| | treated with either an ARB, irbesartan, or an ACE | |
| | inhibitor (ACE-I), captopril. BNP and ANP were | |
| | measured in both subsamples and correlated with | |
| | clinical, functional and neurohormonal parameters | |
| | throughout a follow-up period of six months and at the | |
| | sixth month. RESULTS: The mean EF in the patient | |
| | sample was 33.43 +/− 6.52% and in the controls was | |
| | 61.96 +/− 3.53% (p = 0.000). The mean BNP (pmol/L) in | |
| | patients was 44.78 +/− 54.36 and in the controls was | |
| | 7.12 +/− 8.28 (p = 0.000) and the mean ANP (pmol/L) | |
| | was 30.32 +/− 25.97 in patients and 11.18 +/− 7.92 in | |
| | controls (p = 0.000). A statistically significant difference | |
| | was found between patients and healthy controls. | |
| | Significant correlations were found between natriuretic | |
| | peptides and EF. Between the baseline phase and the | |
| | sixth month, BNP and ANP decreased significantly in | |
| | the ARB group. At the sixth month, both BNP and | |
| | ANP were lower in the ARB group. Evidence of | |
| | clinical benefit was found with both ARB or ACE-I | |
| | treatment throughout the six months, with patients | |
| | moving from classes III and IV to class II NYHA. | |
| | Improvement of EF was also found, with transition of | |
| | patients with lower EF (even <30%) to higher values. | |
| | EF was higher in the ARB group at the sixth month. | |
| diagnosis of left | BNP levels are elevated in asymptomatic or | Diabetes Metab. 2004 |
| ventricular dysfunction | symptomatic left ventricular dysfunction, hypertrophy | Sep; 30(4): 381-6. |
| | and coronary artery disease. | |
| Prediction of | BNP levels are elevated in infants with PPHN but not | Pediatrics. 2004 |
| pulmonary | in infants with other forms of respiratory distress not | Nov; 114(5): 1297- |
| hypertension (PPHN) | associated with PPHN | 304. |
| of term or near-term | | |
| infants with respiratory | | |
| distress | | |
| BNP as a predictor of | In patients with persistent atrial fibrillation, BNP levels | Can J Cardiol. 2004 |
| successful | are associated with successful cardioversion and | Oct; 20(12): 1245-8. |
| cardioversion in | maintenance of sinus rhythm two weeks after | |
| patients with persistent | cardioversion. | |
| atrial fibrillation | | |
| BNP reflects the | BNP was the only biochemical parameter that | Int J Cardiol. 2004 |
| remodelling process in | independently predicted interventricular septal diastolic | Nov; 97(2): 251-6. |
| hypertension. | diameter (p < 0.05), left ventricular mass index (p < 0.01) | |
| | and ratio of the velocity-time integrals of the E and A | |
| | waves of the mitral inflow in a stepwise logistic | |
| | regression analysis (p < 0.05). | |
| predict mortality in | growing evidence supports the hypothesis that BNP | Int J Infect Dis. 2004 |
| septic shock | could be an early predictor of mortality in septic shock | Sep; 8(5): 271-4 |
| Can be used for | A decrease in BNP level of > or = 50% during the first | Congest Heart Fail. |
| predicting response to | 3 months on epoprostenol was strongly predictive of | 2004 Sep- |
| epoprostenol therapy | event-free survival (p = 0.003 | Oct; 10(5): 221-5. |
| in pulmonary arterial | | |
| hypertension | | |
| Can be used as a | Increased NT pro-BNP was closely linked to severity | Am J Cardiol. 2004 |
| marker to evaluate | | Sep 15; 94(6): 740-5. |
| severity of aortic | | |
| stenosis, monitor | | |
| disease progression at | | |
| an early stage, and | | |
| decide on the optimal | | |
| time for aortic valve | | |
| replacement AVR. | | |
| Strong predictor of | B-type natriuretic peptide levels by themselves were | N Engl J Med. 2002 |
| heart failure (HF) in | more accurate than any historical or physical findings | Jul 18; 347(3): 161-7 |
| patients with the chief | or laboratory values in identifying congestive heart | |
| complaint of dyspnea | failure as the cause of dyspnea | |
| BNP ratio before and | BNP is elevated in end-stage renal disease before | Gun Nephrol. 1995 |
| after dialysis could be | dialysis and drops 20-40% after a dialysis session. | Nov; 44 Suppl 1: S61- |
| used as a measure of | | 4 |
| volume reduction and | | |
| resultant decreased left | | |
| ventricular wall | | |
| tension in patients with | | |
| end-stage renal disease | | |
| A rapid assay for BNP | Patients diagnosed with evidence of abnormal LV | Circulation. 2002 Feb |
| can reliably detect the | diastolic function (n = 119) had a mean BNP | 5; 105(5): 595-601 |
| presence of diastolic | concentration of 286 +/− 31 pg/mL; those in the normal | |
| abnormalities on | LV group (n = 175) had a mean BNP concentration of | |
| echocardiography | 33 +/− 3 pg/mL | |
| Screening: can be used | At study entry, plasma BNP and the heart failure | J Am Coil Cardiol. |
| to identify patients | survival score (HFSS) showed a significant correlation | 2001 |
| with heart failure who | (r = −0.706). During follow-up, Kaplan-Meier estimates | Dec; 38(7): 1934-41. |
| have an increased risk | of freedom from clinical events differed significantly | |
| of deterioration of their | for patients above and below the 75th percentile | |
| functional status | concentrations of plasma BNP (p < 0.0001). Changes | |
| | in plasma BNP were significantly related to changes in | |
| | limitations of physical activity, as demonstrated by | |
| | logistic regression analysis (chi-square statistic = 24.9, | |
| | p < 0.0001). Proportional hazards analysis confirmed | |
| | BNP as a powerful predictor of functional status | |
| | deterioration (p < 0.0001). | |
| plasma biomarker for | Elevation of the plasma BNP concentration is more | Mayo Clin Proc. |
| ventricular | specifically related to left ventricular hypertrophy | 2001 |
| hypertrophy in dialysis | compared with the other natriuretic peptides levels in | Nov; 76(11): 1111-9 |
| patients with end-stage | patients with end-stage renal disease independent of | |
| renal disease (ESRD) | congestive heart failure | |
| BNP levels are a | BNP levels, in addition to other neurohormonal, | Circulation. 2002 |
| strong, independent | clinical, and hemodynamic variables, were obtained | May |
| predictor of sudden | from 452 patients with a left ventricular ejection | 21; 105(20): 2392-7 |
| death in patients with | fraction (LVEF) < or = 35%. For prediction of sudden | |
| CHF. | death, only survivors without heart transplantation | |
| | (HTx) or a mechanical assist device and patients who | |
| | died suddenly were analyzed. Up to 3 years, 293 | |
| | patients survived without HTx or a mechanical assist | |
| | device, 89 patients died, and 65 patients underwent | |
| | HTx. Mode of death was sudden in 44 patients (49%), | |
| | whereas 31 patients (35%) had pump failure and 14 | |
| | patients (16%) died from other causes. Univariate risk | |
| | factors of sudden death were log BNP (P = 0.0006), log | |
| | N-terminal atrial natriuretic peptide (P = 0.003), LVEF | |
| | (P = 0.005), log N-terminal BNP (P = 0.006), systolic | |
| | blood pressure (P = 0.01), big endothelin (P = 0.03), and | |
| | NYHA class (P = 0.04). In the multivariate model, log | |
| | BNP level was the only independent predictor of | |
| | sudden death (P = 0.0006). Using a cutoff point of log | |
| | BNP < 2.11 (130 pg/mL), Kaplan-Meier sudden death- | |
| | free survival rates were significantly higher in patients | |
| | below (99%) compared with patients above (81%) this | |
| | cutoff point (P = 0.0001). | |
| Marker for allograft | Higher BNP levels in patients long after heart | Am J Cardiol. 2004 |
| performance after | transplantation are associated with allograft | Aug 15; 94(4): 454-8 |
| heart transplantation | dysfunction and cardiac allograft vasculopathy and are | |
| | strongly and independently predictive of cardiovascular | |
| | death | |
| Can be used as a | 233 individuals with HF risk factors attended an HF | Congest Heart Fail. |
| screening tool to detect | screening sessions - all specifically without a history of | 2003 May- |
| patients progressing | HF. Of the 233 subjects screened, the majority (92%) | Jun; 9(3): 127-32. |
| from stage A to stage | had > or = 1 risk factor with an average of 2.8 risk | |
| B in heart failure | factors for HF. Many subjects also had symptoms | |
| | consistent with HF. A total of 24 subjects (10.3%) had | |
| | a BNP level >100 pg/mL, and a total of 32 subjects | |
| | (13.7%) had a level >80 pg/mL. The follow-up data | |
| | showed that all 24 subjects saw their physician within 6 | |
| | months after the screening. By 12 months following the | |
| | initial screening program, 21 of the 24 subjects with | |
| | elevated BNP levels (88%) underwent further testing | |
| | and 18 of the 24 (67%) had changes in their | |
| | medications. BNP screening identifies subjects at high | |
| | risk for developing HF | |
| Prognostic determinant | NT-proBNP appeared to be more sensitive than | Circulation. 2003 |
| in light-chain | conventional echocardiographic parameters in | May |
| amyloidosis | detecting clinical improvement or worsening of | 20; 107(19): 2440-5. |
| | amyloid cardiomyopathy during follow-up | Epub 2003 Apr 28 |
| Marker for myocarditis | Immunohistochemical analysis of endomyocardial | Int J Cardiol. 1995 |
| | biopsy specimens showed ANP and BNP | Dec; 52(3): 213-22. |
| | immunoreactivity in the early myocarditis group (ANP | |
| | in 4/10 and BNP in 3/10) and the late myocarditis | |
| | group (ANP and BNP in 4/10), but not in the controls | |
| | (0/8). | |
| Marker for assessment | BNP level was increased in non-ST elevation acute | Yonsei Med J. 2004 |
| of myocardial | coronary syndrome patients compared with stable | Apr 30; 45(2): 255-62 |
| ischemia in non-ST | angina patients (133.9 +/− 87.4 vs. 12.2 +/− 9.2 pg/mL, | |
| elevation acute | p < 0.05). | |
| coronary syndrome | | |
| Marker for symptom | Plasma natriuretic peptide levels are elevated in | Circulation. 2003 Apr |
| onset in aortic stenosis | symptomatic patients with aortic stenosis. | 15; 107(14): 1884-90. |
| | Measurement of natriuretic peptides may complement | Epub 2003 Mar 31 |
| | clinical and echocardiographic evaluation of patients | |
| | with aortic stenosis. | |
| Marker for Acute | Plasma N-terminal pro-brain natriuretic peptide is | Eur Respir J. 2003 |
| pulmonary embolism | elevated in the majority of cases of pulmonary | Oct; 22(4): 649-53 |
| | embolism resulting in right ventricular overload. | |
| | Plasma levels reflect the degree of right ventricular | |
| | overload and may help to predict short-term outcome | |
| Assessing the | Plasma concentrations of ANP and BNP in the | Circ J. 2004 |
| myocardial infarction | peripheral blood were measured in 88 asymptomatic | Oct; 68(10): 923-7 |
| size | (New York Heart Association class I) patients with | |
| | previous MI. The infarct size was quantitatively | |
| | calculated from rest thallium-201 myocardial single | |
| | photon emission computed tomography. In multivariate | |
| | linear regression analysis that included MI size, | |
| | hemodynamic parameters, and age as covariables, only | |
| | BNP concentrations had a significant association with | |
| | MI size (p = 0.0001) | |
| Marker for rheumatic | increased plasma BNP levels in patients with rheumatic | Eur J Heart Fail. |
| heart disease | heart disease compared with healthy subjects. | 2004 Oct; 6(6): 757-60 |
| Detection of | Fifty-one patients with valve disease underwent single | Int J Cardiol. 2004 |
| asymptomatic valvular | valve surgery (mitral stenosis, MS, 13; mitral | Jul; 96(1): 21-4. |
| disease, and is a | regurgitation, MR, 16; aortic stenosis, AS, 14; aortic | |
| clinical marker for | regurgitation, AR, 8 patients). Blood samples, | |
| determining the | echocardiographic and cardiac catheterization data | |
| optimal surgical timing | were obtained before operation and echocardiographic | |
| | examination were performed after 1-year of operations. | |
| | RESULTS: In patients subjected to single heart valve | |
| | surgery, plasma BNP mean levels were 214.6 +/− 48.5 | |
| | pg/ml. In plasma BNP levels, there was only | |
| | significant difference between MS and AS group (MS | |
| | 67.5 +/− 9.7 vs. AS 314.3 +/− 112.0 pg/ml, P = 0.04). There | |
| | were no relationships between plasma BNP levels and | |
| | pre-operative cardiac functions. After 1-year of the | |
| | valve surgery, NYHA functional class was reduced in | |
| | 36 patients (70.6%) and plasma BNP levels before the | |
| | surgery significantly correlated with post-operative | |
| | NYHA functional class | |
| Marker for | plasma ANP and BNP levels could be markers for | Med Pediatr Oncol. |
| cardiotoxicity of drugs | doxorubicin-induced cardiotoxicity in children | 2001 Jul; 37(1): 4-9. |
| Marker for chronic | plasma BNP or ANP level may be a useful indicator | Respir Med. 1999 |
| respiratory failure with | for detecting the presence of cor pulmonale in patients | Jul; 93(7): 507-14 |
| cor pulmonale | with chronic respiratory failure. | |
| Marker for lung cancer | Human small cell lung cancer cells produce brain | Oncology. |
| | natriuretic peptide. | 1999; 56(2): 155-9 |
| Marker for adrenal | immunoreactive BNP is present both in normal human | Eur J Endocrinol. |
| related tumors | adrenal glands and in adrenal tumors. Multiple | 1996 Sep; 135(3): 352- |
| | molecular forms of BNP were found to be present in | 6 |
| | the tumor tissues of pheochromocytoma and | |
| | aldosteronoma | |

According to other optional embodiments of the present invention, variants of this cluster according to the present invention (amino acid and/or nucleic acid sequences of HUMNATPEP) may optionally have one or more of the following utilities, some of which are related to utilities described above. It should be noted that these utilities are optionally and preferably suitable for human and non-human animals as subjects, except where otherwise noted.

A non-limiting example of such a utility is the detection of coronary artery disease (in conjunction with stress testing, such that the subject undergoes cardiac stress testing, during or after which BNP is detected and/or measured, quantitatively or qualitatively), optionally and preferably including risk assessment and stratification. For example, the method could optionally be performed as follows: measuring a baseline level of a marker related to BNP in the subject; inducing a cardiac stress in the subject; measuring the marker related to BNP level immediately post cardiac stress; and calculating a relative change in the marker related to BNP level; wherein coronary artery disease is detected in the subject if the relative change in marker related to BNP level after cardiac stress is greater than a predetermined clinically effective threshold value. Optionally and preferably, risk assessment/stratification may be performed according to the relative change; the higher the relative change, the greater the risk (and presumably also the more severe the coronary artery disease). Cardiac stress may optionally be induced through exercise stress testing, for example, and/or pharmacologic stress testing (for example through the administration of dobutamine).

Optionally, the test may be performed in a subject with no previous history of cardiac disease, and/or in a subject with one or risk factors for cardiac disease, including but not limited to, age greater than 35 years, history of smoking, diabetes mellitus, obesity, high blood pressure, high cholesterol, elevated low density lipoproteins and family history of cardiac disease.

Use of the known protein, BNP, for such a diagnostic utility has been described in US Patent Application No. 20040243010, hereby incorporated by reference as if fully set forth herein.

Another non-limiting example of such a utility is the diagnosis of acute coronary syndrome, for example related to some type of myocardial injury, optionally and preferably by performing a method of diagnosing myocardial ischemia and/or myocardial necrosis in a subject, the method comprising: determining a level of BNP variant according to the present invention (a HUMNATPEP protein, protein fragment, oligonucleotide or fragment thereof) in a sample obtained from the subject; and correlating the level of BNP variant to the presence or absence of myocardial ischemia in the subject. The term “acute coronary syndromes” (“ACS”) has been applied to a group of coronary disorders that result from ischemic insult to the heart, also referred to as myocardial injury or myocardial damage, that is commonly secondary to atherosclerosis or hypertension, and is the leading cause of death in the United States. ACS is commonly caused by occlusion associated with coronary artery disease cause by atherosclerotic plaque formation and progression to either further occlusion or fissure. ACS can be manifested as stable angina, unstable angina, or myocardial infarction.

The terms “ischemia and ischemic” relate to damage to the myocardium as a result of a reduction of blood flow to the heart. The terms “angina pectoris”, “stable angina”, “unstable angina”, “silent ischemia” are generally related to myocardial ischemia. One skilled in the art will recognize these terms, which are described in “The Merck Manual of Diagnosis and Therapy” Seventeenth Edition, 1999, Ed. Keryn A. G. Lane, pp. 1662-1668, incorporated by reference as if fully set forth herein. The term ischemia is also related to what one skilled in the art would consider as minor myocardial injury or damage. The term ischemia is further described in the Journal of the American College of Cardiology 36, 959-969 (2000), incorporated by reference as if fully set forth herein.

The terms “necrosis and necrotic” relate to myocardial cell death as a result of a reduction or stoppage of blood flow to the heart. Myocardial necrosis is a condition of the heart which is more severe than myocardial ischemia. The term “myocardial infarction” is generally related to myocardial necrosis. One skilled in the art will recognize these terms, which are described in “The Merck Manual of Diagnosis and Therapy” Seventeenth Edition, 1999, Ed. Keryn A. G. Lane, pp. 1668-1677, incorporated by reference as if fully set forth herein. The term necrosis is also related to what one skilled in the art would consider as major myocardial injury or damage. The terms myocardial infarction and necrosis are further described in the Journal of the American College of Cardiology 36, 959-969 (2000), incorporated by reference as if fully set forth herein.

This method may optionally be performed concurrently with or following stress testing (described above). Correlating may optionally be performed (for example) by comparing the variant BNP level to a threshold variant BNP level, whereby, when the variant BNP level exceeds the threshold variant BNP level, the subject is diagnosed as having myocardial ischemia and/or myocardial necrosis. Optionally and preferably, the method is able to distinguish between myocardial ischemia and myocardial necrosis.

Use of the known protein, BNP, for such a diagnostic utility has been described in US Patent Application No. 20030109420, hereby incorporated by reference as if fully set forth herein.

Another non-limiting example of such a utility is the method of diagnosing cardiac transplant rejection, optionally including predicting and/or detecting such rejection, in a subject, by using a BNP variant according to the present invention. Optionally and preferably, a plurality of measurements may be made over time, for example to monitor a subject undergoing cardiac transplant.

Use of the known protein, BNP, for such a diagnostic utility has been described in U.S. Pat. No. 6,117,644, hereby incorporated by reference as if fully set forth herein.

Cluster HUMNATPEP encodes for variants of BNP, which belongs to a family of proteins that includes ANP and CNP. These proteins have a number of functions as described below for CRP (and as described elsewhere herein for ANP and variants thereof according to the present invention). These functions may optionally relate to one or more diagnostic utilities of variants of BNP (HUMNATPEP polypeptides and fragments, peptides, oligonucleotides and fragments and any other variant biomarker related thereto).

CNP mRNA is also found in the vascular endothelium, consistent with the peptide's putative autocrine/paracrine role in the regulation of vascular tone and cell growth (Komatsu et al., 1992; Furuya et al., 1993). Several cytokines, including transforming growth factor-h, interleukin-1a, tumor necrosis factor-a, and endotoxin, stimulate CNP mRNA expression (Yamamoto et al., 1997). CNP is more potent than ANP in eliciting smooth muscle relaxation but is a less potent inducer of diuresis and natriuresis (Sudoh et al., 1990; Clavell et al., 1993). Thus, in the cardiovascular system, CNP is likely to have primary local roles in the blood vessel wall rather than as a circulating natriuretic hormone (Komatsu et al., 1992). The 22-amino acid fragment is the mature and more active form and is expressed in the nervous system and endothelial cells (Ogawa et al., 1992; Espiner et al., 1995; Suzuki et al., 2001).

Table 53b below describes diagnostic utilities for the cluster HUMNATPEP that were found through microarrays, including the statistical significance thereof and a reference. One or more HUMNATPEP variants according to the present invention may optionally have one or more of these utilities.

TABLE 53b

| | Statistical | |
|---|---|---|
| Diagnostic utility | significance | reference |
| Gene over expressed in transitional cell | 0.006 | Ramaswamy S; Golub TR PNAS (2001) |
| carcinoma (vs. normal bladder), therefore | | Multiclass cancer diagnosis using tumor |
| could be used for the diagnosis of bladder | | gene expression signatures. |
| cancer | | |
| Staging and grading; detection of | 2.6e⁻⁶, 3.4e⁻⁴ | Dyrskjot L, Orntoft TF Nat Genet (2003) |
| advanced bladder tumors (stage Ta vs. T1 | respectively | Identifying distinct classes of bladder |
| & T2+, grade 2 vs. grade 3), over | | carcinoma using microarrays. |
| expression in the advanced tumors. | | |
| Gene over expressed in renal cell | 0.007 | Ramaswamy S; Golub TR PNAS (2001) |
| carcinoma (vs. normal) | | Multiclass cancer diagnosis using tumor |
| | | gene expression signatures. |
| Identification of thymus dysfunction, gene | | GNF database |
| over expressed in naive mature | | (http://www.ncbi.nlm.nih.gov/projects/geo/): |
| thymocytes (vs. immature thymocytes) | | GBS785, probe 206801_at |

Other non-limiting exemplary utilities for HUMNATPEP variants according to the present invention are described in greater detail below and also with regard to the previous section on clinical utility.

The heart-selective diagnostic marker prediction engine provided the following results with regard to cluster HUMNATPEP. Predictions were made for selective expression of transcripts of this contig in heart tissue, according to the previously described methods. The numbers on the y-axis of the first figure below refer to weighted expression of ESTs in each category, as “parts per million” (ratio of the expression of ESTs for a particular cluster to the expression of all ESTs in that category, according to parts per million).

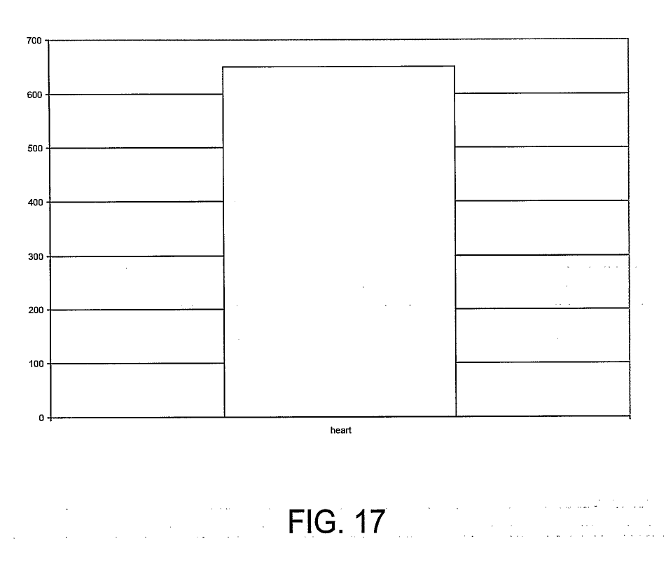

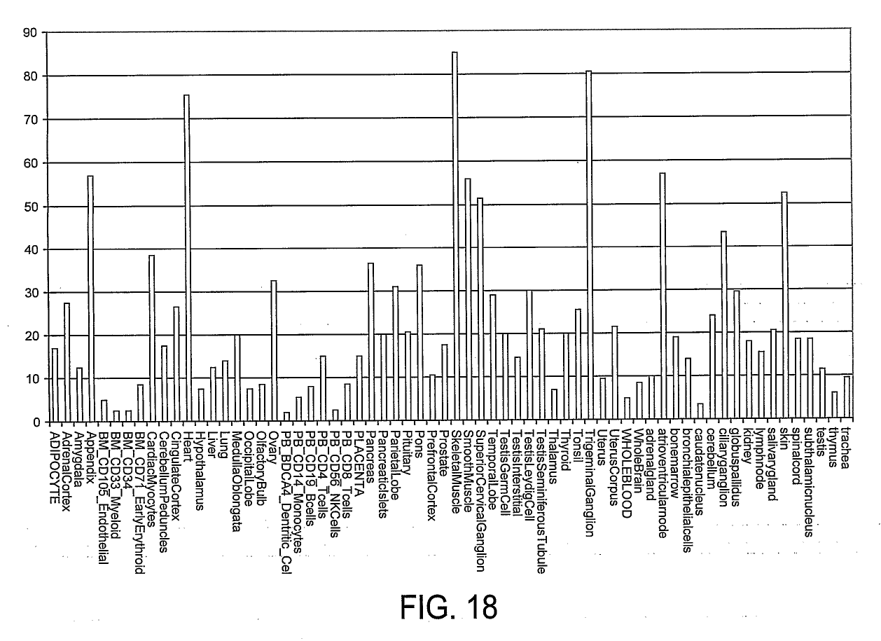

Overall, the following results were obtained as shown with regard to the histogram in FIG. 17, concerning the number of heart-specific clones in libraries/sequences; as well as with regard to the histogram in FIG. 18, concerning the actual expression of oligonucleotides in various tissues, including heart.

This cluster was found to be selectively expressed in heart for the following reasons: in a comparison of the ratio of expression of the cluster in heart specific ESTs to the overall expression of the cluster in non-heart ESTs, which was found to be 17.3; the ratio of expression of the cluster in heart specific ESTs to the overall expression of the cluster in muscle-specific ESTs which was found to be 351.5; and fisher exact test P-values were computed both for library and weighted clone counts to check that the counts are statistically significant, and were found to be 8.20E-17.

One particularly important measure of specificity of expression of a cluster in heart tissue is the previously described comparison of the ratio of expression of the cluster in heart as opposed to muscle. This cluster was found to be specifically expressed in heart as opposed to non-heart ESTs as described above. However, many proteins have been shown to be generally expressed at a higher level in both heart and muscle, which is less desirable. For this cluster, as described above, the ratio of expression of the cluster in heart specific ESTs to the overall expression of the cluster in muscle-specific ESTs which was found to be 17.3, which clearly supports specific expression in heart tissue.

As noted above, cluster HUMNATPEP features 4 transcript(s), which were listed in Table 49 above. These transcript(s) encode for protein(s) which are variant(s) of protein Natriuretic peptides B precursor (SEQ ID NO: 138) [Contains: Gamma-brain natriuretic peptide; Brain natriuretic peptide 32 (BNP-32)]. A description of each variant protein according to the present invention is now provided.

Variant protein HUMNATPEP_PEA_{—}1_P2 (SEQ ID NO:139) according to the present invention has an amino acid sequence; it is encoded by transcript(s) HUMNATPEP_PEA_{—}1_T1 (SEQ ID NO:127). An alignment is given to the known protein (Natriuretic peptides B precursor (SEQ ID NO: 138) [Contains: Gamma-brain natriuretic peptide; Brain natriuretic peptide 32 (BNP-32)]) at the end of the application. One or more alignments to one or more previously published protein sequences are given in the alignment table located on the attached CDROM. A brief description of the relationship of the variant protein according to the present invention to each such aligned protein is as follows:

Comparison report between HUMNATPEP_PEA_{—}1_P2 (SEQ ID NO:139) and ANFB_HUMAN (SEQ ID NO:138):

1. An isolated chimeric polypeptide encoding for HUMNATPEP_PEA_{—}1_P2 (SEQ ID NO:139), comprising a first amino acid sequence being at least 90% homologous to MDPQTAPSRALLLLLFLHLAFLGGRSHPLGSPGSASDLETSGLQEQRNHLQGKLSELQVEQTSLEPL QESPRPTGVWKSREVATEGIRGHRKMVLYTLRAPRSPKMVQGSGCFGRKMDRISSSSGLGCK corresponding to amino acids 1-129 of ANFB_HUMAN (SEQ ID NO:138), which also corresponds to amino acids 1-129 of HUMNATPEP_PEA_{—}1_P2 (SEQ ID NO:139), and a second amino acid sequence being at least 70%, optionally at least 80%, preferably at least 85%, more preferably at least 90% and most preferably at least 95% homologous to a polypeptide having the sequence GKHPLPPRPPSPIPVCDTVRVTLGFVVSGNHTL (SEQ ID NO: 640) corresponding to amino acids 130-162 of HUMNATPEP_PEA_{—}1_P2 (SEQ ID NO:139), wherein said first amino acid sequence and second amino acid sequence are contiguous and in a sequential order.

2. An isolated polypeptide encoding for a tail of HUMNATPEP_PEA_{—}1_P2 (SEQ ID NO:139), comprising a polypeptide being at least 70%, optionally at least about 80%, preferably at least about 85%, more preferably at least about 90% and most preferably at least about 95% homologous to the sequence GKHPLPPRPPSPIPVCDTVRVTLGFVVSGNHTL (SEQ ID NO: 640) in HUMNATPEP_PEA_{—}1_P2 (SEQ ID NO:139).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The variant protein has the following domains, as determined by using InterPro. The domains are described in Table 54:

TABLE 54
InterPro domain(s)

| | | | Position(s) |
|---|---|---|---|
| InterPro ID | Domain description | Analysis type | on protein |
| IPR000663 | Natriuretic peptide | FPrintScan | 109-118, |
| | | | 118-127 |
| IPR002408 | Natriuretic peptide, | FPrintScan | 11-27, 110-120, |
| | brain type | | 120-133, 28-38, |
| | | | 43-61 |
| IPR000663 | Natriuretic peptide | HMMPfam | 46-128 |
| IPR000663 | Natriuretic peptide | HMMSmart | 105-128 |
| IPR000663 | Natriuretic peptide | ScanRegExp | 112-128 |
| IPR002408 | Natriuretic peptide, | BlastProDom | 27-129 |
| | brain type | | |

Variant protein HUMNATPEP_PEA_{—}1_P2 (SEQ ID NO:139) is encoded by the following transcript(s): HUMNATPEP_PEA_{—}1_T1 (SEQ ID NO:127). The coding portion of transcript HUMNATPEP_PEA_{—}1_T1 (SEQ ID NO:127) starts at position 249 and ends at position 734.

Variant protein HUMNATPEP_PEA_{—}1_P3 (SEQ ID NO:140) according to the present invention has an amino acid sequence; it is encoded by transcript(s) HUMNATPEP_PEA_{—}1_T2 (SEQ ID NO:128). An alignment is given to the known protein (Natriuretic peptides B precursor (SEQ ID NO: 138) [Contains: Gamma-brain natriuretic peptide; Brain natriuretic peptide 32 (BNP-32)]) at the end of the application. One or more alignments to one or more previously published protein sequences are given in the alignment table located on the attached CDROM. A brief description of the relationship of the variant protein according to the present invention to each such aligned protein is as follows:

Comparison report between HUMNATPEP_PEA_{—}1_P3 (SEQ ID NO:140) and ANFB_HUMAN (SEQ ID NO:138):

1. An isolated chimeric polypeptide encoding for HUMNATPEP_PEA_{—}1_P3 (SEQ ID NO:140), comprising a first amino acid sequence being at least 90% homologous to MDPQTAPSRALLLLLFLHLAFLGGRSHPLGSPGSASDLETSGLQ corresponding to amino acids 1-44 of ANFB_HUMAN (SEQ ID NO:138), which also corresponds to amino acids 1-44 of HUMNATPEP_PEA_{—}1_P3 (SEQ ID NO:140), and a second amino acid sequence being at least 70%, optionally at least 80%, preferably at least 85%, more preferably at least 90% and most preferably at least 95% homologous to a polypeptide having the sequence VRAEGSSGGLDSSNERVLTCCPKRPSSFLWN (SEQ ID NO: 641) corresponding to amino acids 45-75 of HUMNATPEP_PEA_{—}1_P3 (SEQ ID NO:140), wherein said first amino acid sequence and second amino acid sequence are contiguous and in a sequential order.

2. An isolated polypeptide encoding for a tail of HUMNATPEP_PEA_{—}1_P3 (SEQ ID NO:140), comprising a polypeptide being at least 70%, optionally at least about 80%, preferably at least about 85%, more preferably at least about 90% and most preferably at least about 95% homologous to the sequence VRAEGSSGGLDSSNERVLTCCPKRPSSFLWN (SEQ ID NO: 641) in HUMNATPEP_PEA_{—}1_P3 (SEQ ID NO:140).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The variant protein has the following domains, as determined by using InterPro. The domains are described in Table 55:

TABLE 55
InterPro domain(s)

| | | | Position(s) |
|---|---|---|---|
| InterPro ID | Domain description | Analysis type | on protein |
| IPR002408 | Natriuretic peptide, brain type | FPrintScan | 11-27, |
| | | | 28-38 |
| IPR002408 | Natriuretic peptide, brain type | BlastProDom | 27-54 |
| IPR002408 | Natriuretic peptide, brain type | FPrintScan | 11-27, |
| | | | 28-38 |
| IPR002408 | Natriuretic peptide, brain type | BlastProDom | 27-54 |

Variant protein HUMNATPEP_PEA_{—}1_P3 (SEQ ID NO:140) is encoded by the following transcript(s): HUMNATPEP_PEA_{—}1_T2 (SEQ ID NO: 128). The coding portion of transcript HUMNATPEP_PEA_{—}1_T2 (SEQ ID NO:128) starts at position 249 and ends at position 473.

Variant protein HUMNATPEP_PEA_{—}1_P7 (SEQ ID NO:141) according to the present invention has an amino acid sequence; it is encoded by transcript(s) HUMNATPEP_PEA_{—}1_T4 (SEQ ID NO:130). An alignment is given to the known protein (Natriuretic peptides B precursor (SEQ ID NO: 138) [Contains: Gamma-brain natriuretic peptide; Brain natriuretic peptide 32 (BNP-32)]) at the end of the application. One or more alignments to one or more previously published protein sequences are given in the alignment table located on the attached CDROM. A brief description of the relationship of the variant protein according to the present invention to each such aligned protein is as follows:

Comparison report between HUMNATPEP_PEA_{—}1_P7 (SEQ ID NO:141) and ANFB_HUMAN (SEQ ID NO:138):

1. An isolated chimeric polypeptide encoding for HUMNATPEP_PEA_{—}1_P7 (SEQ ID NO:141), comprising a first amino acid sequence being at least 90% homologous to MVLYTLRAPRSPKMVQGSGCFGRKMDRISSSSGLGCKVLRRH corresponding to amino acids 93-134 of ANFB_HUMAN (SEQ ID NO:138), which also corresponds to amino acids 1-42 of HUMNATPEP_PEA_{—}1_P7 (SEQ ID NO:141).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

The variant protein has the following domains, as determined by using InterPro. The domains are described in Table 56:

TABLE 56
InterPro domain(s)

| | | | Position(s) on |
|---|---|---|---|
| InterPro ID | Domain description | Analysis type | protein |
| IPR000663 | Natriuretic peptide | FPrintScan | 17-26, 26-35 |
| IPR002408 | Natriuretic peptide, brain | FPrintScan | 18-28, 28-41 |
| | type | | |
| IPR000663 | Natriuretic peptide | HMMPfam | 1-36 |
| IPR000663 | Natriuretic peptide | HMMSmart | 13-36 |
| IPR000663 | Natriuretic peptide | ScanRegExp | 20-36 |
| IPR002408 | Natriuretic peptide, brain | BlastProDom | 1-42 |
| | type | | |

Variant protein HUMNATPEP_PEA_{—}1_P7 (SEQ ID NO:141) is encoded by the following transcript(s): HUMNATPEP_PEA_{—}1_T4 (SEQ ID NO:130). The coding portion of transcript HUMNATPEP_PEA_{—}1_T4 (SEQ ID NO:130) starts at position 257 and ends at position 382.

Table 57 below describes the starting and ending position of HUMNATPEP_PEA_{—}1_node_{—}2 (SEQ ID NO:133) on each of the relevant transcripts. Experimental results for this segment are described below.

TABLE 57
Segment location on transcripts

| | Segment | Segment |
|---|---|---|
| Transcript name | starting position | ending position |
| HUMNATPEP_PEA_1_T2 | 381 | 612 |
| (SEQ ID NO: 128) | | |
| HUMNATPEP_PEA_1_T3 | 381 | 612 |
| (SEQ ID NO: 129) | | |

Table 58 below describes the starting and ending position of HUMNATPEP_PEA_{—}1_node_{—}3 (SEQ ID NO:134) on each of the relevant transcripts. Experimental results for this segment are described below.

TABLE 58
Segment location on transcripts

| | Segment | Segment |
|---|---|---|
| Transcript name | starting position | ending position |
| HUMNATPEP_PEA_1_T1 (SEQ | 381 | 508 |
| ID NO: 127) | | |
| HUMNATPEP_PEA_1_T2 (SEQ | 613 | 740 |
| ID NO: 128) | | |
| HUMNATPEP_PEA_1_T3 (SEQ | 613 | 740 |
| ID NO: 129) | | |

Table 59 below describes the starting and ending position of HUMNATPEP_PEA_{—}1_node_{—}4 (SEQ ID NO:135) on each of the relevant transcripts. Experimental results for this segment are described below.

TABLE 59
Segment location on transcripts

| | Segment | Segment |
|---|---|---|
| Transcript name | starting position | ending position |
| HUMNATPEP_PEA_1_T1 (SEQ | 509 | 636 |
| ID NO: 127) | | |
| HUMNATPEP_PEA_1_T2 (SEQ | 741 | 868 |
| ID NO: 128) | | |
| HUMNATPEP_PEA_1_T3 (SEQ | 741 | 868 |
| ID NO: 129) | | |
| HUMNATPEP_PEA_1_T4 (SEQ | 241 | 368 |
| ID NO: 130) | | |

Table 60 below describes the starting and ending position of HUMNATPEP_PEA_{—}1_node_{—}5 (SEQ ID NO:136) on each of the relevant transcripts. Experimental results for this segment are described below.

TABLE 60
Segment location on transcripts

| | Segment | Segment |
|---|---|---|
| Transcript name | starting position | ending position |
| HUMNATPEP_PEA_1_T1 (SEQ | 637 | 1178 |
| ID NO: 127) | | |
| HUMNATPEP_PEA_1_T3 (SEQ | 869 | 1410 |
| ID NO: 129) | | |

Expression of Homo sapiens natriuretic peptide precursor B (NPPB) HUMNATPEP transcripts which are detectable by amplicon as depicted in sequence name HUMNATPEP seg3-4WT (SEQ ID NO: 144) specifically in heart tissue

Expression of Homo sapiens natriuretic peptide precursor B (NPPB) transcripts detectable by or according to seg3-4 node, HUMNATPEP seg3-4WT (SEQ ID NO: 144) amplicon and primers HUMNATPEP seg3-4WT-F (SEQ ID NO: 142) and HUMNATPEP seg3-4WT-R (SEQ ID NO: 143) was measured by real time PCR (this transcript relates to the known protein, or “WT” protein). In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44-46, Table 7, “Tissue samples in normal panel” above), to obtain a value of expression for each sample relative to median of the heart samples.

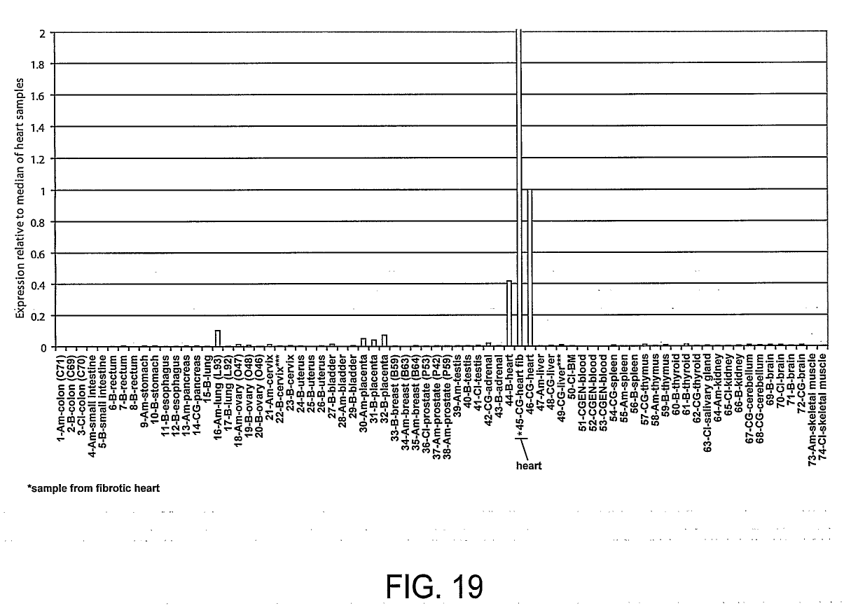

FIG. 19 is a histogram showing relative expression of the above-indicated Homo sapiens natriuretic peptide precursor B (NPPB) known protein transcripts in heart tissue samples as opposed to other tissues.

As is evident from FIG. 19, the expression of Homo sapiens natriuretic peptide precursor B (NPPB) transcripts detectable by the above amplicon in heart tissue samples was higher than in the other samples (Sample Nos. 44-46 Table 7, “Tissue samples in normal panel”). Note that the expression of the above amplicon in one of the heart samples, sample no. 45, was higher compared to its expression in the other two heart samples (sample 44 and 46). Sample no. 45 is from fibrotic heart, and samples 44 and 46 are samples from normal hearts.

Primer pairs are also optionally and preferably encompassed within the present invention; for example, for the above experiment, the following primer pair was used as a non-limiting illustrative example only of a suitable primer pair: HUMNATPEP seg3-4WT-F (SEQ ID NO: 142) forward primer; and HUMNATPEP seg3-4WT-R (SEQ ID NO: 143) reverse primer.

The present invention also preferably encompasses any amplicon obtained through the use of any suitable primer pair; for example, for the above experiment, the following amplicon was obtained as a non-limiting illustrative example only of a suitable amplicon: HUMNATPEP seg3-4WT (SEQ ID NO: 144).

Forward primer HUMNATPEP seg3-4WT-F
(SEQ ID NO:142):
GTCCGGGTTACAGGAGCAGC
Reverse primer HUMNATPEP seg3-4WT-R
(SEQ ID NO:143):
CCGCCTCAGCACTTTGCAG
Amplicon HUMNATPEP seg3-4WT (SEQ ID NO:144):
GTCCGGGTTACAGGAGCAGCGCAACCATTTGCAGGGCAAACTGTCGGAGC
TGCAGGTGGAGCAGACATCCCTGGAGCCCCTCCAGGAGAGCCCCCGTCCC
ACAGGTGTCTGGAAGTCCCGGGAGGTAGCCACCGAGGGCATCCGTGGGCA
CCGCAAAATGGTCCTCTACACCCTGCGGGCACCACGAAGCCCCAAGATGG
TGCAAGGGTCTGGCTGCTTTGGGAGGAAGATGGACCGGATCAGCTCCTCC
AGTGGCCTGGGCTGCAAAGTGCTGAGGCGG

Expression of ANFB_HUMAN (SEQ ID NO:138) Natriuretic peptide HUMNATPEP transcripts which are detectable by amplicon as depicted in sequence name HUMNATPEP seg2 (SEQ ID NO: 147) specifically in heart tissue:

Expression of ANFB_HUMAN (SEQ ID NO:138) Natriuretic peptide transcripts detectable by or according to seg2 node, HUMNATPEPseg2 (SEQ ID NO: 147) amplicon and HUMNATPEPseg2F2 (SEQ ID NO: 145), HUMNATPEPseg2R2 (SEQ ID NO: 146) primers was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44-46, Table 7, “Tissue samples in normal panel” above), to obtain a value of expression for each sample relative to median of the heart.

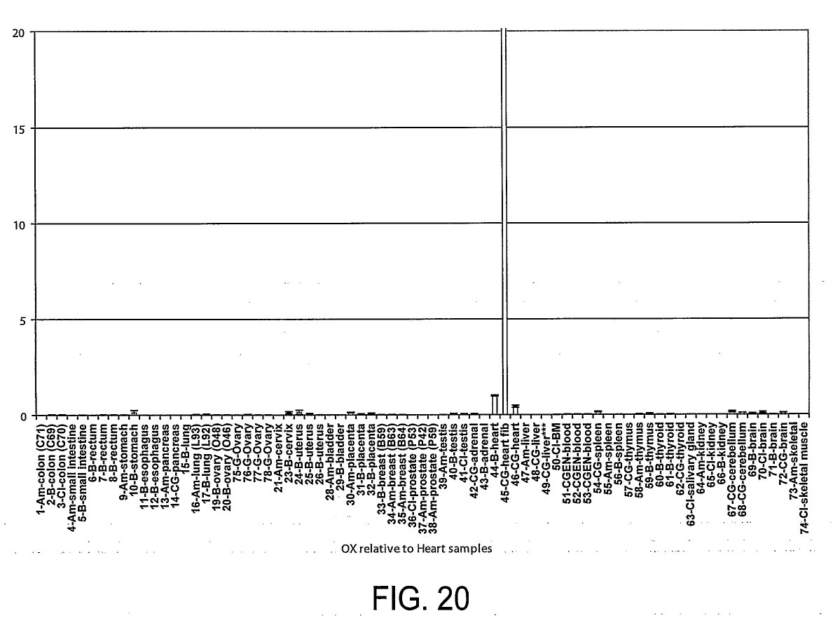

FIG. 20 is a histogram showing relative expression of the above-indicated ANFB_HUMAN (SEQ ID NO:138) Natriuretic peptides transcripts in heart tissue samples as opposed to other tissues.

As is evident from FIG. 20, the expression of ANFB_HUMAN (SEQ ID NO:138) Natriuretic peptide transcripts detectable by the above amplicon in heart tissue samples was higher than in most of the other samples (Sample Nos. 1-26, 28-43, 47-74 Table 7, “Tissue samples in normal panel”). Note that the expression of the above amplicon in one of the heart samples, sample no. 45, was higher compared to its expression in the other two heart samples (sample 44 and 46). Sample no. 45 is from fibrotic heart, and samples 44 and 46 are samples from normal hearts.

Primer pairs are also optionally and preferably encompassed within the present invention; for example, for the above experiment, the following primer pair was used as a non-limiting illustrative example only of a suitable primer pair: HUMNATPEPseg2F2 (SEQ ID NO: 145) forward primer; and HUMNATPEPseg2R2 (SEQ ID NO: 146) reverse primer.

The present invention also preferably encompasses any amplicon obtained through the use of any suitable primer pair; for example, for the above experiment, the following amplicon was obtained as a non-limiting illustrative example only of a suitable amplicon: HUMNATPEPseg2 (SEQ ID NO: 147).

| | |
|---|---|
| Forward primer HUMNATPEPseg2F2: | (SEQ ID NO: 145) |
| GCAGCAATGAAAGGGTCCTC | |
| Reverse primer HUMNATPEPseg2R2: | (SEQ ID NO: 146) |
| CATGGCACCCAAGTGAACC | |
| Amplicon HUMNATPEPseg2: | (SEQ ID NO: 147) |

GCAGCAATGAAAGGGTCCTCACCTGCTGTCCCAAGAGGCCCTCATCTTTCCTTTGGAATTAGT
GATAAAGGAATCAGAAAATGGAGAGACTGGGTGCCCTGACCCTGTACCCAAGGCAGTCGGTT
CACTTGGGTGCCATG

Expression of ANFB_HUMAN (SEQ ID NO:138) Natriuretic peptides HUMNATPEP transcripts which are detectable by amplicon as depicted in sequence name HUMNATPEPseg5 (SEQ ID NO: 150) specifically in heart tissue:

Expression of ANFB_HUMAN (SEQ ID NO:138) Natriuretic peptides transcripts detectable by or according to seg5 node, HUMNATPEPseg5 (SEQ ID NO: 150) amplicon and HUMNATPEPseg5F (SEQ ID NO: 148), HUMNATPEPseg5R (SEQ ID NO:149) primers was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicons was normalized to the geometric mean of the quantities of the housekeeping genes. The normalized quantity of each RT sample was then divided by the median of the quantities of the heart tissue samples (Sample Nos. 44-46, Table 7 above, Tissue samples in normal panel), to obtain a value of expression for each sample relative to median of the heart tissue.

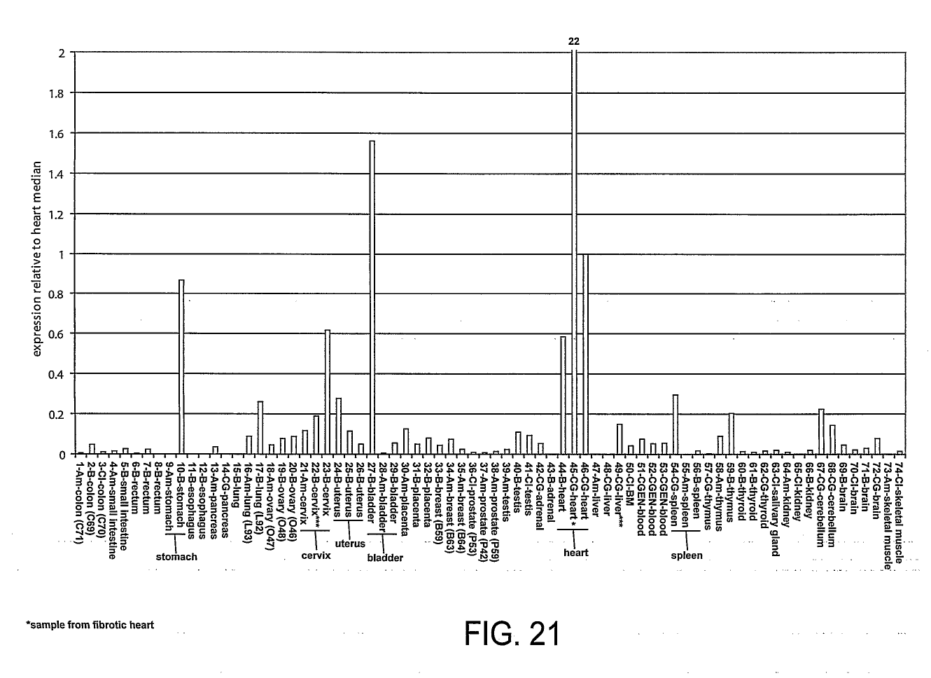

FIG. 21 is a histogram showing relative expression of the above-indicated ANFB_HUMAN (SEQ ID NO:138) Natriuretic peptides transcripts in heart tissue samples as opposed to other tissues.

As is evident from FIG. 21, the expression of ANFB_HUMAN (SEQ ID NO:138) Natriuretic peptides transcripts detectable by the above amplicon in heart tissue samples was significantly higher than in most other samples (Sample Nos. 1-9, 11-22, 24-26, 28-43, 47-74 Table 7, “Tissue samples in normal panel” above). Note that the expression of the above amplicon in one of the heart samples, sample no. 45, was higher compared to its expression in the other two heart samples (sample 44 and 46). Sample no. 45 is from fibrotic heart, and samples 44 and 46 are samples from normal hearts.

Primer pairs are also optionally and preferably encompassed within the present invention; for example, for the above experiment, the following primer pair was used as a non-limiting illustrative example only of a suitable primer pair: HUMNATPEPseg5 forward primer (SEQ ID NO: 148); and HUMNATPEPseg5 reverse primer (SEQ ID NO: 149).

The present invention also preferably encompasses any amplicon obtained through the use of any suitable primer pair; for example, for the above experiment, the following amplicon was obtained as a non-limiting illustrative example only of a suitable amplicon: HUMNATPEPseg5 (SEQ ID NO: 150).

| | |
|---|---|
| HUMNATPEPseg5 Forward primer: | (SEQ ID NO: 148) |
| CTTCCCCCATTCCAGTGTGT | |
| HUMNATPEPseg5 Reverse primer: | (SEQ ID NO: 149) |
| GAGGAAGCGATGTCCAGGTG | |
| HUMNATPEPseg5 | (SEQ ID NO: 150) |
| Amplicon: | |

CTTCCCCCATTCCAGTGTGTGACACTGTTAGAGTCACTTTGGGGTTTGTTGTCTCTGGGAACCA
CACTCTTTGAGAAAAGGTCACCTGGACATCGCTTCCTC

### Description for Cluster HUMCDDANF

Cluster HUMCDDANF features 3 transcript(s) and 10 segment(s) of interest, the names for which are given in Tables 61 and 62, respectively. The selected protein variants are given in table 63.

TABLE 61
Transcripts of interest
Transcript Name

HUMCDDANF_PEA_1_T6 (SEQ ID NO: 151)
HUMCDDANF_PEA_1_T7 (SEQ ID NO: 152)
HUMCDDANF_PEA_1_T8 (SEQ ID NO: 153)

TABLE 62
Segments of interest

Segment Name

| | |
|---|---|
| HUMCDDANF_PEA_1_node_2 (SEQ ID NO: 154) | HUMCDDANF_PEA_1_T6 (SEQ ID |
| | NO: 151), HUMCDDANF_PEA_1_T7 |
| | (SEQ ID NO: 152) and |
| | HUMCDDANF_PEA_1_T8 (SEQ ID |
| | NO: 153) |
| HUMCDDANF_PEA_1_node_4 (SEQ ID NO: 155) | HUMCDDANF_PEA_1_T7 (SEQ ID |
| | NO: 152) and HUMCDDANF_PEA_1_T8 |
| | (SEQ ID NO: 153) |
| HUMCDDANF_PEA_1_node_5 (SEQ ID NO: 156) | HUMCDDANF_PEA_1_T6 (SEQ ID |
| | NO: 151), HUMCDDANF_PEA_1_T7 |
| | (SEQ ID NO: 152) and |
| | HUMCDDANF_PEA_1_T8 (SEQ ID |
| | NO: 153) |
| HUMCDDANF_PEA_1_node_6 (SEQ ID NO: 157) | HUMCDDANF_PEA_1_T6 (SEQ ID |
| | NO: 151) and HUMCDDANF_PEA_1_T8 |
| | (SEQ ID NO: 153) |
| HUMCDDANF_PEA_1_node_7 (SEQ ID NO: 158) | HUMCDDANF_PEA_1_T6 (SEQ ID |
| | NO: 151) and HUMCDDANF_PEA_1_T8 |
| | (SEQ ID NO: 153) |
| HUMCDDANF_PEA_1_node_8 (SEQ ID NO: 159) | HUMCDDANF_PEA_1_T6 (SEQ ID |
| | NO: 151), HUMCDDANF_PEA_1_T7 |
| | (SEQ ID NO: 152) and |
| | HUMCDDANF_PEA_1_T8 (SEQ ID |
| | NO: 153) |
| HUMCDDANF_PEA_1_node_10 (SEQ ID NO: 160) | HUMCDDANF_PEA_1_T6 (SEQ ID |
| | NO: 151), HUMCDDANF_PEA_1_T7 |
| | (SEQ ID NO: 152) and |
| | HUMCDDANF_PEA_1_T8 (SEQ ID |
| | NO: 153) |
| HUMCDDANF_PEA_1_node_3 (SEQ ID NO: 161) | HUMCDDANF_PEA_1_T6 (SEQ ID |
| | NO: 151), HUMCDDANF_PEA_1_T7 |
| | (SEQ ID NO: 152) and |
| | HUMCDDANF_PEA_1_T8 (SEQ ID |
| | NO: 153) |
| HUMCDDANF_PEA_1_node_11 (SEQ ID NO: 162) | HUMCDDANF_PEA_1_T6 (SEQ ID |
| | NO: 151), HUMCDDANF_PEA_1_T7 |
| | (SEQ ID NO: 152) and |
| | HUMCDDANF_PEA_1_T8 (SEQ ID |
| | NO: 153) |
| HUMCDDANF_PEA_1_node_12 (SEQ ID NO: 163) | HUMCDDANF_PEA_1_T6 (SEQ ID |
| | NO: 151), HUMCDDANF_PEA_1_T7 |
| | (SEQ ID NO: 152) and |
| | HUMCDDANF_PEA_1_T8 (SEQ ID |
| | NO: 153) |

TABLE 63
Proteins of interest

| Protein Name | Corresponding Transcript(s) |
|---|---|
| HUMCDDANF_PEA_1_P6 (SEQ ID NO: 165) | HUMCDDANF_PEA_1_T6 (SEQ ID NO: 151) |
| HUMCDDANF_PEA_1_P9 (SEQ ID NO: 166) | HUMCDDANF_PEA_1_T7 (SEQ ID NO: 152); |
| | HUMCDDANF_PEA_1_T8 (SEQ ID NO: 153) |

These sequences are variants of the known protein Atrial natriuretic factor precursor (SEQ ID NO:164) (SwissProt accession identifier ANF_HUMAN (SEQ ID NO:164); known also according to the synonyms ANF; Atrial natriuretic peptide; ANP; Prepronatriodilatin; CDP), referred to herein as the previously known protein.

Protein Atrial natriuretic factor precursor (SEQ ID NO:164) is known or believed to have the following function(s): Atrial natriuretic factor (ANF) is a potent vasoactive substance synthesized in mammalian atria and is thought to play a key role in cardiovascular homeostasis. Has a cGMP-stimulating activity.

A-type natriuretic peptide (ANP) (also referred to as atrial natriuretic peptide, atrial natriuretic factor (ANF) or cardiodilatin (Forssmann et al Histochem Cell Biol 110: 335-357, 1998) is a 28 amino acid peptide that is synthesized, stored, and released atrial myocytes in response to atrial distension, angiotensin II stimulation, endothelin, and sympathetic stimulation (beta-adrenoceptor mediated). ANP is synthesized as a precursor molecule (pro-ANP) that is converted to an active form, ANP, by proteolytic cleavage and also forming N-terminal ANP (1-98). N-terminal ANP and ANP have been reported to increase in patients exhibiting atrial fibrillation and heart failure (Rossi et al. Journal of the American College of Cardiology 35: 1256-62, 2000). In addition to atrial natriuretic peptide (ANP99-126) itself, linear peptide fragments from its N-terminal prohormone segment are known; such fragments are also expected to occur in ANP variants according to the present invention. As the skilled artisan will recognize, however, because of its relationship to ANP variant, the concentration of N-terminal ANP variant molecule can also provide diagnostic or prognostic information in patients. The phrase “marker related to ANP variant or ANP variant related peptide” refers to any polypeptide that originates from the pro-ANP variant molecule (1-126), or a variant thereof. Proteolytic degradation of ANP variant and of peptides related to ANP variant may occur and these proteolytic fragments are also encompassed it the term “ANP variant related peptides.”

Elevated levels of ANP are found during hyperyolemia, atrial fibrillation and congestive heart failure. ANP is involved in the long-term regulation of sodium and water balance, blood volume and arterial pressure. This hormone decreases aldosterone release by the adrenal cortex, increases glomerular filtration rate (GFR), produces natriuresis and diuresis (potassium sparing), and decreases renin release thereby decreasing angiotensin II. These actions contribute to reductions in blood volume and therefore central venous pressure (CVP), cardiac output, and arterial blood pressure. Several isoforms of ANP have been identified, and their relationship to stroke incidence studied. See, e.g., Rubatu et al., Circulation 100:1722-6, 1999; Estrada et al., Am. J. Hypertens. 7:1085-9, 1994.

Chronic elevations of ANP appear to decrease arterial blood pressure primarily by decreasing systemic vascular resistance. The mechanism of systemic vasodilation may involve ANP receptor-mediated elevations in vascular smooth muscle cGMP as well as by attenuating sympathetic vascular tone. This latter mechanism may involve ANP acting upon sites within the central nervous system as well as through inhibition of norepinephrine release by sympathetic nerve terminals. ANP may be viewed as a counter-regulatory system for the renin-angiotensin system.

Known polymorphisms for this sequence are as shown in Table 64.

TABLE 64
Amino acid mutations for Known Protein

| SNP position(s) on amino | |
|---|---|
| acid sequence | Comment |
| 32 | V -> M (in dbSNP: 5063). |
| | /FTId = VAR_014579. |
| 152-153 | Missing (in isoform 2). |
| | /FTId = VAR_000594. |
| 65 | E -> D |

Protein Atrial natriuretic factor precursor (SEQ ID NO:164) localization is believed to be Secreted.

It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Aldosterone antagonist; Diuretic; Electrolyte absorption agonist. A therapeutic role for a protein represented by the cluster has been predicted. The cluster was assigned this field because there was information in the drug database or the public databases (e.g., described herein above) that this protein, or part thereof, is used or can be used for a potential therapeutic indication: Antihypertensive, diuretic; Antiasthma; Urological; Cardiostimulant.

The following GO Annotation(s) apply to the previously known protein. The following annotation(s) were found: physiological processes; blood pressure regulation, which are annotation(s) related to Biological Process; hormone, which are annotation(s) related to Molecular Function; and extracellular, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

It should be noted that a number of diagnostic utilities are described for variants of HUMCDDANF herein; these utilities may also optionally be applied to various forms of these variants, including but not limited to, propeptides, propeptides after cleavage, biologically active peptides, and/or fragments of these variants (optionally including degradation products of these variants).

According to optional but preferred embodiments of the present invention, variants of this cluster according to the present invention (amino acid and/or nucleic acid sequences of HUMCDDANF) may optionally have one or more of the following utilities, as described with regard to the Table 65 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 65
Utilities for Variants of HUMCDDANF, related to ANP (Atrial Natriuretic Peptide/Factor,
ANF, NPPA, Prepronatriodilatin, Cardiodilatin-related peptide, CDP, ANF_HUMAN
(SEQ ID NO: 164), ANP_HUMAN)

| Diagnostic entity | Rationale | Reference |
|---|---|---|
| Administration in | In a subgroup of patients with | Vervoort et al., Am |
| diagnostic test of | uncomplicated type 1 diabetes, an increase | J Kidney Dis. 2002 |
| vascular dysfunction | in glomerular permselectivity can be | Jul; 40(1): 9-15. |
| (such as in diabetes | unmasked by the infusion of ANP. | |
| mellitus) | | |
| Assessing and | Treatment of hypertension with perindopril | Yalcin et al., Clin |
| monitoring | reduces plasma atrial natriuretic peptide | Cardiol. 2000 |
| antihypertensive therapy | levels, left ventricular mass, and improves | Jun; 23(6): 437-41 |
| | echocardiographic parameters of diastolic | |
| | function. | |
| Assessing volume | Regional stress but not myocarditis itself is | Benvenuti et al., Int |
| overload (myocardial | probably responsible for ventricular ANP | J Cardiol. 2003 |
| compromise) and | expression in myocarditis. | Mar; 88(1): 57-61 |
| prognosis in Chagas | | |
| disease (chronic | | |
| chagasic | | |
| cardiomyopathy) | | |
| Assessing volume | ANP levels are a good indicator of volume | Kula et al., Can J |
| overload in Rheumatic | overload. Drugs such as angiotensin- | Cardiol. 2003 Mar |
| Cardiac disease | converting enzyme inhibitors should be | 31; 19(4): 405-8 |
| | introduced at an early stage of rheumatic | |
| | MR because heart failure may progress | |
| | silently, even if patients are taking digoxin. | |
| Assessment of dry | Assessment of dry weight in haemodialysis | Wolfram et al., |
| weight in haemodialysis | patients by the volume markers ANP and | Nephrol Dial |
| patients | cGMP. | Transplant. 1996; 11 |
| | | Suppl 2: 28-30 |
| Diagnosis and assessing | Measurement of plasma atrial natriuretic | Weir et al., Acta |
| treatment of patent | peptide concentration has a role in predicting | Paediatr. 1992 |
| ductus arteriosus | when indomethacin treatment is indicated. | Sep; 81(9): 672-5 |
| Diagnosis and prognosis | Plasma BNP (pmol/L) and ANP (pmol/L) | Falcao et al., J |
| of CHF, cardiac | were determined in 68 hypertensive patients | Renin Angiotensin |
| hypertrophy and/or MI | with dilated cardiomyopathy, NYHA class | Aldosterone Syst. |
| | III-IV and ejection fraction (EF) < or =40%, | 2004 Sep; 5(3): 121-9; |
| | and in 26 normal controls. The patient group | Squire et al., Clin |
| | was randomly subdivided into two | Sci (Lond). 2004 |
| | subgroups of 34 patients, each treated with | Sep; 107(3): 309-16 |
| | either an angiotensin receptor blocker | |
| | (ARB), irbesartan, or an ACE inhibitor | |
| | (ACE-I), captopril. RESULTS: The mean | |
| | EF in the patient sample was 33.43 +/− 6.52% | |
| | and in the controls was 61.96 +/− 3.53% | |
| | (p = 0.000). The mean BNP (pmol/L) in | |
| | patients was 44.78 +/− 54.36 and in the | |
| | controls was 7.12 +/− 8.28 (p = 0.000) and the | |
| | mean ANP (pmol/L) was 30.32 +/− 25.97 in | |
| | patients and 11.18 +/− 7.92 in controls | |
| | (p = 0.000). A statistically significant | |
| | difference was found between patients and | |
| | healthy controls. Significant correlations | |
| | were found between natriuretic peptides and | |
| | EF. Between the baseline phase and the sixth | |
| | month, BNP and ANP decreased | |
| | significantly in the ARB group. At the sixth | |
| | month, both BNP and ANP were lower in | |
| | the ARB group. Evidence of clinical benefit | |
| | was found with both ARB and ACE-I | |
| | treatment throughout the six months, with | |
| | patients moving from classes III and IV to | |
| | class II NYHA. Improvement of EF was also | |
| | found, with transition of patients with lower | |
| | EF (even <30%) to higher values. EF was | |
| | higher in the ARB group at the sixth month. | |
| | Diagnosis using both N-ANP and N-BNP | |
| | identifies a greater number of patients at risk | |
| | of death or heart failure than either peptide | |
| | alone | |
| Diagnosis of alcohol | Although mean ANP levels increased | Kiefer et al., Acta |
| withdrawal state | significantly in alcoholics between days 1 | Psychiatr Scand. |
| | and 14, they remained diminished compared | 2002 Jan; 105(1): 65-70. |
| | to controls. Individuals in a subgroup of | Kovacs GL. Curr |
| | alcoholics with decreased ANP levels during | Med Chem. 2003 |
| | withdrawal were found to have significantly | Dec; 10(23): 2559-76. |
| | elevated scores for mean and maximum | |
| | craving and a trend to an elevated self-rated | |
| | anxiety on day 14. | |
| | In an early phase of acute withdrawal, | |
| | plasma levels of atrial natriuretic peptide | |
| | (ANP) are high. In patients with delirium | |
| | tremens (DT) elevated levels of ANP were | |
| | observed days before the actual onset of DT. | |
| | It is concluded that the altered plasma ANP | |
| | secretion might be associated with, and | |
| | therefore used as, an indicator of the onset of | |
| | DT. | |
| Diagnosis of and | High levels of 5-HIAA excretion and plasma | Zuetenhorst et al., |
| volume overload and | ANP were found to be associated with | Cancer. 2003 Apr |
| prognosis assessment in | carcinoid heart disease. | 1; 97(7): 1609-15 |
| carcinoid heart disease | | |
| Diagnosis of arrhythmia | N-ANP levels were normalized following | Erbay et al., Clin |
| and/or its risk | septal closure in most patients, except in | Sci (Lond). 2004 |
| | those with atrial fibrillation attacks | Sep; 107(3): 297-302 |
| | following corrective surgery. | |
| Diagnosis of brain | A statistically significant increase in the | Nogami et al., |
| infarction | number of ANP-immunoreactive glial cells | Histochem J. 2001 |
| | (mainly astrocytes) was observed in the | Feb; 33(2): 87-90 |
| | white matter surrounding the brain infarction | |
| | compared with the intact area. | |
| Diagnosis of cardiac | Cardiac rhabdomyomas exhibit a fetal | Benvenuti et al., |
| rhabdomyoma | pattern of atrial natriuretic peptide | Exp Mol Pathol. |
| | immunoreactivity. | 2001 Feb; 70(1): 65-9 |
| Diagnosis of | A direct contributory role of ANP in the | Rubattu et al., J |
| cerebrovascular | development of hypertension and of | Hypertens. 2001 |
| disorders or their risk | cerebrovascular disorders has been | Nov; 19(11): 1923-31 |
| | suggested by the use of molecular genetic | |
| | approaches | |
| Diagnosis of | Quantitative RT-PCR analysis showed a | Rubattu et al., J |
| Doxorubicin | selective 5-fold increase of ANP mRNA in | Hypertens. 2001 |
| cardiotoxicity | Dox-treated dog hearts in comparison to | Nov; 19(11): 1923-31 |
| | controls. Similarly, northern analysis gave a | |
| | selective 4.5-fold increase in ANP | |
| | transcripts in Dox-treated rat hearts. On the | |
| | other hand, there was a selective decrease | |
| | (approximately 39%) of ANP transcripts in | |
| | Dox-treated cardiac cultures relative to | |
| | controls. | |
| Diagnosis of early | ANP but not BNP appears to be a sensitive | Bayerle-Eder et al., |
| diastolic dysfunction | biochemical marker for early diastolic | Horm Metab Res. |
| | dysfunction in Type 1 diabetes. | 2003 |
| | | May; 35(5): 301-7 |
| Diagnosis of fetal | ANF levels in amniotic fluid and in maternal | Di Lieto et al., J |
| cardiac malformations | venous blood are increased early in the case | Matern Fetal |
| | of fetuses with cardiac malformations, with | Neonatal Med. 2002 |
| | or without associated karyotype alteration. | Mar; 11(3): 183-7 |
| | Chromosomally abnormal fetuses without | |
| | heart malformations have normal ANF | |
| | levels. | |
| Diagnosis of gastric | Atrial natriuretic peptide (ANP) is present in | Gower et al., Am J |
| pathology including | gastric mucosa. | Physiol Gastrointest |
| gastric dilatation and | | Liver Physiol. 2003 |
| gastric cancer | | Apr; 284(4): G638-45 |
| Diagnosis of low venous | Formerly preeclamptic women with a | Aardenburg et al., J |
| capacitance states, such | subnormal plasma volume differ from | Soc Gynecol |
| as may be tendency for | controls with a normal plasma volume by a | Investig. 2005 |
| preeclampsia | reduced venous capacitance. During volume | Feb; 12(2): 107-11 |
| | loading, patients differed from controls by a | |
| | larger rise in alpha-ANP, pulse rate, and | |
| | cardiac output, and by a lower estimated | |
| | venous capacitance. | |
| Diagnosis of | Immunohistochemical analysis of | Takemura et al., Int |
| myocarditis | endomyocardial biopsy specimens showed | J Cardiol. 1995 |
| | ANP and BNP immunoreactivity in the early | Dec; 52(3): 213-22 |
| | myocarditis group (ANP in 4/10 and BNP in | |
| | 3/10) and the late myocarditis group (ANP | |
| | and BNP in 4/10), but not in the controls | |
| | (0/8). | |
| Diagnosis of paroxysmal | During the onset period of Supraventricular | Schiffrin et al., N |
| atrial tachycardia | Tachycardia, plasma ET (endothelin) and | Engl J Med. 1985 |
| | ANP were markedly elevated and 30 | May |
| | minutes after its termination they were | 2; 312(18): 1196-7.; |
| | lowered significantly, but their | |
| | concentrations were still 2-fold higher than | |
| | those of the control group. | |
| Diagnosis of perinatal | Perinatal hypoxia causes ventricular | Hohimer et al., |
| hypoxia | enlargement associated with increased atrial | High Alt Med Biol. |
| | natriuretic peptide (ANP) mRNA levels in | 2003 |
| | newborn mice. | Summer; 4(2): 241-54 |
| Diagnosis of | ANP could be involved in the neovascular | Rollin et al., Mol |
| proliferative diabetic | and fibrotic complications of proliferative | Vis. 2004 Jul |
| retinopathy or its risk | diabetic retinopathy (PDR): ANP was | 15; 10: 450-7 |
| | immunohistochemically localized in the | |
| | epiretinal membranes of patients with PDR. | |
| | Vitreous ANP concentrations were | |
| | significantly higher in patients with active | |
| | PDR compared to patients with quiescent | |
| | PDR, diabetes without PDR or controls | |
| | <0.05. Significant differences were also | |
| | observed between vitreous ANP levels in | |
| | diabetic patients without PDR and control | |
| | subjects. There was no significant | |
| | correlation between serum and vitreous ANP | |
| | levels in any of the patient groups. | |
| Diagnosis of diabetic | Diabetic pregnancy is associated with lower | Rollin et al., Mol |
| pregnancy | levels of ANP compared to non-diabetic | Vis. 2004 Jul |
| | pregnancy. Levels of ANP (p = 0.03) are | 15; 10: 450-7 |
| | significantly lower in diabetes than in non- | |
| | diabetes subjects throughout pregnancy and | |
| | postpartum. | |
| Diagnosis of pulmonary | Atrial natriuretic peptide and cGMP levels | Wiedemann et al., J |
| hypertension | were increased about tenfold and fivefold | Am Coll Cardiol. |
| | compared with controls in both primary and | 2001 |
| | nonprimary pulmonary hypertension. | Oct; 38(4): 1130-6 |
| Diagnosis of radiation | Circulating levels of ANP were measured in | Wondergem et al., J |
| mediated cardiac | patients who have been irradiated on a large | Am Coll Cardiol. |
| dysfunction | part of the heart (50-80%; Hodgkin's | 2001 |
| | disease) or smaller part of the heart (20-30%; | Oct; 38(4): 1130-6 |
| | primary breast cancer). RESULTS: | |
| | ANP plasma levels of 121 patients | |
| | (Hodgkin's disease, 73 patients; breast | |
| | cancer, 48 patients) and 67 controls were | |
| | examined. ANP plasma levels of both | |
| | Hodgkin patients (28.8 +/− 2.2, P = 0.003) and | |
| | breast cancer patients (20.4 +/− 2.8 ng/l, | |
| | P = 0.01) were significantly elevated when | |
| | compared to age-matched controls (13.5 +/− | |
| | 1.2 ng/l). Patients with clinical symptoms of | |
| | cardiovascular disease (n = 25) had | |
| | significantly higher ANP plasma levels | |
| | (P < 0.001) compared to patients in the same | |
| | treatment group without evidence of cardiac | |
| | disease (50.2 +/− 7.5 vs. 23.3 +/− 1.3 ng/l, | |
| | P < 0.001, and 38.2 +/− 12.4 vs. 16.3 +/− 1.6 ng/l, | |
| | P < 0.001, for Hodgkin's disease and | |
| | breast cancer, respectively). Eight patients | |
| | suffered from essential hypertension (n = 8), | |
| | whereas the remaining group of 17 patients | |
| | showed a variety of cardiac disorders (i.e. | |
| | myocardial infarction, decreasing ventricular | |
| | function, and atrial fibrillations). | |
| Diagnosis of salt | ProANP(1-30) correlated with salt | Melander et al., |
| sensitive hypertension | sensitivity at baseline (r = 0.76, P < 0.000001), | 2002 |
| | after the low-(r = 0.80, P < 0.0000001) and | May; 39(5): 996-9 |
| | high-salt diets (r = 0.85, P < 0.00000001). | |
| | Changes in mean blood pressure (deltaMBP) | Kato N. et al. |
| | were significantly correlated with changes in | Hypertens Res. |
| | atrial natriuretic peptide during salt loading | 2002 |
| | (r = −0.34, p = 0.0018). | Nov; 25(6): 801-9. |
| Diagnosis of small cell | A tumor cell line from a patient with small | Johnson et al., |
| lung carcinoma | cell lung carcinoma and hyponatremia was | Cancer. 1997 Jan |
| | able ectopically to produce, process, and | 1; 79(1): 35-44 |
| | secrete ANP in the same immunoreactive | |
| | form as the biologically active molecule. | |
| | Preliminary studies show that tumor cell line | |
| | NCI-H1284 contains an enzyme that can | |
| | cleave precursors at the same amino acid | |
| | sequences needed to produce ANP-(S99- | |
| | Y126) from pro-ANP. | |
| Differential diagnosis of | Chronic administration of atrial natriuretic | Lee et al., J Androl. |
| male infertility | peptide reduces testosterone production of | 2003 Nov |
| | testes in mice. | Dec; 24(6): 912-7 |
| Differentiation of recent | An inverse relation was demonstrated | van den Berg et al., |
| atrial fibrillation from | between the duration of AF (atrial | Europace. 2004 |
| prolonged atrial | fibrillation) and plasma ANP concentration. | Sep; 6(5): 433-7 |
| fibrillation. Predicting | In addition, a reduced ANP response to | |
| cardioversion and/or | exercise has been shown to be predictive of | |
| maze operation success. | unsuccessful cardioversion of AF to sinus | |
| Measuring atrial | rhythm. Finally, ANP has also been shown | |
| degeneration. | to predict outcome after a maze operation. | |
| | Outcome was poor when preoperative | |
| | plasma ANP concentration was low. | |
| | Moreover, high atrial collagen content, as a | |
| | measure of atrial degeneration, correlated | |
| | with low ANP. These data indicate that ANP | |
| | may serve as a marker of atrial integrity, | |
| | which may help in selecting AF patients for | |
| | therapeutic interventions. | |
| Monitoring menstrual | The strongest ANP immunostaining was | Ivanova et al., |
| cycle and/or differential | observed in granulosa cells obtained from | Reprod Biol. 2003 |
| diagnosis of female | large follicles. The ANP immunostaining | Jul; 3(2): 173-81. |
| infertility | detected by Mab 5D3 had granular | |
| | appearance moderately expressed in the | |
| | submembrane region of granulosa cells of all | |
| | types of follicles. | |
| Prognosis in patients on | Plasma levels of the vasoactive peptides | Odar-Cederlof et |
| hemodialysis | ANP and NPY are the most important group | al., ASAIO J. 2003 |
| | in a hierarchy of variables that predict | Jan-Feb; 49(1): 74-80 |
| | imminent death in hemodialysis patients, | |
| | and NPY is associated with late death. ANP | |
| | and NPY apparently sum up the detrimental | |
| | influence of many factors in hemodialysis | |
| | patients. | |
| Prognostic marker in | Pro-atrial natriuretic peptide is a prognostic | Morgenthaler et al., |
| sepsis | marker in sepsis, similar to the APACHE II | Crit Care. 2005 |
| | score. | Feb; 9(1): R37-45. |
| | | Epub 2004 Dec 17. |
| Protection against | Preconditioning of livers with ANP | Gerwig et al., J |
| hepatic preservation | markedly reduces hepatic ischemia- | Hepatol. 2003 |
| injury in liver | reperfusion injury. Preconditioning with | Sep; 39(3): 341-8 |
| transplantation process. | both ANP and 8-Br-cGMP significantly | |
| Diagnosis of such injury | reduced caspase-3-like activity and the | |
| and/or a tendency | number of triphosphate nick-end labeling- | |
| thereto. | positive cells. After ischemia, degenerative | |
| | cell changes were clearly reduced in ANP | |
| | pretreated livers. After reperfusion, ANP | |
| | preconditioning led to a significant reduction | |
| | of necrotic hepatocytes and endothelial cells | |
| | in periportal zones. Cell proliferation was | |
| | not affected. | |
| Treatment and | Pretreatment with atrial natriuretic peptide | Strohle et al., Am J |
| prevention of panic | resulted in significantly lower Acute Panic | Psychiatry. 2001 |
| attack and anxiety | Inventory scores than pretreatment with | Sep; 158(9): 1514-6 |
| disorders. Diagnosis of | placebo. | |
| such panic attacks | Atrial natriuretic peptide (ANP) is causally | Strohle A. |
| and/or anxiety disorders, | involved in sodium lactate-induced panic | Pharmacopsychiatry |
| and/or a tendency | attacks. Furthermore, preclinical and clinical | 2003 Nov; 36 |
| thereto. | data on its anxiolytic activity suggest that | Suppl 3: S207-14. |
| | non-peptidergic ANP receptor ligands may | |
| | be of potential use in the treatment of | |
| | anxiety disorders. | |
| Treatment and/or | The suprarenal abdominal aortic cross- | Mitaka et al., Crit |
| prevention of renal | clamping during aortic aneurysm repair | Care Med. 2003 |
| failure. Including during | causes renal dysfunction after surgery. | Aug; 31(8): 2205-10 |
| surgery, such as aortic | Prophylactic continuous ANP infusion | |
| dissection repair. | limited adverse changes after aortic cross- | |
| Diagnosis of such renal | clamping in urine volume, renal blood flow, | |
| failure or a tendency | and creatinine clearance and serum | |
| thereto. | creatinine concentrations. | |
| Relation to obesity, | Atrial natriuretic peptide (ANP) is a lipolytic | Moro et al., |
| optionally including | agent on isolated human fat cells. | Metabolism. 2005 |
| particular type(s) of | | Jan; 54(1): 122-31 |
| underlying condition(s) | | |
| associated with obesity | | |
| ANP possesses anti- | ANP and its receptors have been shown to | Kiemer AK, Ann |
| inflammatory activity | be expressed and differentially regulated in | Rheum Dis. 2001 |
| and so may be a marker | the immune system, so it has been suggested | Nov; 60 Suppl |
| for inflammation | that ANP has immunomodulatory potency. | 3: iii68-70. |
| | Specifically, ANP was shown to reduce the | |
| | secretion of inflammatory mediators in | |
| | macrophages. | |

According to other optional embodiments of the present invention, variants of this cluster according to the present invention (amino acid and/or nucleic acid sequences of HUMCDDANF) may optionally have one or more of the following utilities, some of which are related to utilities described above. It should be noted that these utilities are optionally and preferably suitable for human and nonhuman animals as subjects, except where otherwise noted.

A non-limiting example of such a utility is diagnosis of stroke and cerebral injury. Optionally and preferably, a plurality of blood pressure related markers is used, including any combination of two or more of ANP (known protein and/or corresponding oligonucleotides), ANP variants according to the present invention (amino acid and/or nucleic acid sequences of HUMCDDANF), BNP (known protein and/or corresponding oligonucleotides) or BNP variants according to the present invention (amino acid and/or nucleic acid sequences of HUMNATPEP). Preferably, the combination includes at least one ANP variant and/or at least one BNP variant according to the present invention.

Optionally, the combination may include one or more markers selected from the group consisting of specific markers of neural tissue injury, markers related to coagulation and hemostasis, and markers related to inflammation, and markers related to apoptosis. These markers may optionally include one or more of VEGF or a variant thereof (as described for example in U.S. Pat. No. 6,783,954, hereby incorporated by reference as if fully set forth herein) or CRP or a variant thereof as described herein with regard to cluster HSCREACT.

Use of the known protein, ANP, for diagnosis of stroke and cerebral injury has been described in US Patent Application No. 20040219509, hereby incorporated by reference as if fully set forth herein.

According to other preferred embodiments of the present invention, there is provided another non-limiting example of such a utility, related to a method of characterizing a risk of future cerebral vasospasm in a subject suffering from a subarrachnoid hemorrhage, comprising: determining the presence or amount of a plurality of subject-derived markers in a sample obtained from the subject, wherein the plurality of markers are independently selected from the group consisting of specific markers of neural tissue injury, markers related to blood pressure regulation, markers related to inflammation, and markers related to apoptosis; and correlating the presence or amount of the plurality of markers to the risk of a future cerebral vasospasm in the subject.

Use of the known protein, ANP, for such a diagnostic utility has been described in US Patent Application No. 2004-0209307, hereby incorporated by reference as if fully set forth herein.

According to still other preferred embodiments of the present invention, there is provided another non-limiting example of such a utility, related to a diagnosis of sepsis, optionally and preferably by a pro-hormone or propeptide of HUMCDDANF (ANP variants) according to the present invention. Alternatively or additionally, the marker may be a prohormone or propeptide of HUMNATPEP (BNP variants) according to the present invention. Such a method optionally and preferably comprises a method for the differential-diagnostic early detection and detection, for the assessment of the severity, and for the assessment of the success of a therapeutic treatment of sepsis and severe infections, in particular sepsis-like systematic infections, characterized in that the content of at least one peptide prohormone variant as described herein and/or of a partial peptide derived therefrom, which is not the mature hormone obtainable from the peptide prohormone, is determined in a sample of a biological fluid of a patient, and the presence of a sepsis or sepsis-like systematic infection, its severity and/or the success of a therapeutic treatment are determined from the detected presence and/or amount of the determined peptide prohormone.

Optionally and preferably, the determination of the prohormone variant and of partial peptides derived therefrom in a serum or plasma of a patient in whom there is a risk of sepsis and in whom symptoms typical of sepsis are found is a valuable diagnostic aid for early detection, i.e. for the detection of infections which may lead to sepsis, and their differentiation from noninfectious etiologies, for the detection of the severity and for the assessment of the success of a treatment of sepsis and sepsis-like systemic infections. The determination is also valuable for diagnosis to distinguish symptoms attributable to systemic microbial infections from other symptoms of noninfectious etiology which, owing to their clinical picture, might suggest a sepsis but in reality are not attributable to a systemic microbial infection, for example from symptoms attributable to noninfectious inflammations of individual organs, to postoperative rejection reactions or cancers. Furthermore, systemic inflammations can be distinguished from local ones.

Use of the known proteins, ANP or BNP, for such a diagnostic utility has been described in US Patent Application No. 20040180396, hereby incorporated by reference as if fully set forth herein.

Yet another non-limiting example of such a utility includes predicting, detecting and monitoring treatment of cardiomyopathies and myocarditis with ANP variants and/or BNP variants according to the present invention as described herein, optionally including one or more of known ANP and/or known BNP. According to preferred embodiments of the present invention, there is provided a method of diagnosing or detecting cardiomyopathies or myocarditis in a patient following an infection. The method comprises obtaining a sample of a biological fluid from the patient, and determining the level of a ANP variant, a BNP variant, ANP, BNP or a fragment thereof, or a combination thereof (but including at least one variant according to the present invention), within the sample of body fluid. The current invention also relates to the monitoring of treatment of cardiomyopathies or myocarditis as a result of an infection, by determining the levels of these proteins and/or fragments, and/or related oligonucleotides of such variants and/or known proteins, at one or more than period prior to and optionally subsequent to, treatment. Multiple samples may optionally be taken over time to assess the effect of treatment, for example

Use of the known proteins, ANP or BNP, for such a diagnostic utility has been described in US Patent Application No. 20040132013, hereby incorporated by reference as if fully set forth herein.

Yet another non-limiting example of such a utility includes diagnosis of dyspnea, chest pain, and/or neurologic dysfunction, and/or differential diagnosis between systolic heart failure and diastolic heart failure, and/or differential diagnosis between atrial fibrillation and congestive heart failure, and/or between atrial fibrillation and myocardial infarction, using an ANP variant and/or a BNP variant according to the present invention. Optionally, one or more of vasopressin, endothelin-2, calcitonin gene related peptide, calcitonin, urotensin 2, and angiotensin 2 may be used in addition for differential diagnosis between systolic heart failure and diastolic heart failure. Optionally, one or more of free cardiac troponin I, free cardiac troponin T, cardiac troponin I in a complex comprising one or both of troponin T and troponin C, cardiac troponin T in a complex comprising one or both of troponin I and troponin C, total cardiac troponin I, total cardiac troponin T, and myoglobin may be used in addition for differential diagnosis between atrial fibrillation and myocardial infarction.

Use of the known proteins, ANP or BNP, for such a diagnostic utility has been described in US Patent Application No. 20040121343, hereby incorporated by reference as if fully set forth herein.

Yet another non-limiting example of such a utility includes diagnosis of a vascular disease including cardiovascular, stroke, pulmonary, renovascular, cerebrovascular, thrombotic or generalized arterial or venous condition or event including acute coronary syndrome (including but not limited to acute myocardial infarction, heart failure, atheromoma or a thrombotic condition), using an ANP variant and/or a BNP variant according to the present invention. Optionally, one or more of myoglobin, myosin light chain (MLC), myosin heavy chain (MHQ, total creatine kinase (CK) including CK-MB, lactate dehydrogenase (LDHH4), aspartate aminotransferase (AST), cardiac troponin I and T (cTn-1 and cTn-T, respectively) and cTn-1 and cTn-1 RNA, fatty acid binding protein (FAB protein) including FABP1 and human heart-type, glycogen phosphorylase-BB isoenzyme, a-atrial natriuretic peptide (ANP), cytoplasmic FABP, brain natriuretic peptide (BNP), adrenomedullin (ADM), low density lipoprotein (LDL), very low density lipoprotein (VLDL), high density lipoprotein (HDL) and intermediate density lipoprotein (11DL), C reactive protein (CRP), serum amyloid A, P-selectin, prostaglandins, platelet-activating factor (PAF), histamine, tumor necrosis factor a (TNFa), soluble TNF receptor 2 (sTNFR2), fibrin, fibrinogen, fibronolytic peptides, modified haemoglobin (HbAlc), ferritin, soluble intercellular adhesion molecule (ICAM) including soluble intercellular adhesion molecule-1 (ICAM1), heat shock proteins, apoB, apoA, apoE, homocysteine or parts thereof, Streptococcus sp., Porphyromonas gingivalis, Helicobacter pylori and Chlamydia pneumoniae or immunological relatives thereof, necrosis and platelet markers, leptin, vasopeptidase inhibitor of cardiac endogenous kinins, heparin, metalloproteinase-9, metalloproteinase-1 including its tissue inhibitor, angiotensin-converting enzyme, CD95/Apol/Fas, hepatocyte-63 growth factor, soluble vascular cell adhesion molecule-1 (VCAM1), plasma brain natriuretic peptide, angiotensin II type receptor, endothelial constitutive nitric oxide synthase, glycoprotein IIE genetic polymorphisms, factor V11a, thrombin, endothelin-1, cardiac myofibrillar proteins, Fas and Fas ligand, ligands thereof or binding partners thereof may be used in addition for diagnosis of such a vascular disease.

Use of the known proteins, ANP or BNP, for such a diagnostic utility has been described in PCT Application No. WO 0223191, hereby incorporated by reference as if fully set forth herein.

Yet another non-limiting example of such a utility includes diagnosis of cardiac decompensation risks, preferably in a method for identifying the risk of onset of cardiac decompensation, using an ANP variant and/or a BNP variant according to the present invention.

Use of the known proteins, ANP or BNP, for such a diagnostic utility has been described in PCT Application No. WO 03035907, hereby incorporated by reference as if fully set forth herein. Other non-limiting exemplary utilities for HUMCDDANF variants according to the present invention are described in greater detail below and also with regard to the previous section on clinical utility.

The heart-selective diagnostic marker prediction engine provided the following results with regard to cluster HUMCDDANF. Predictions were made for selective expression of transcripts of this contig in heart tissue, according to the previously described methods. The numbers on the y-axis of the first figure below refer to weighted expression of ESTs in each category, as “parts per million” (ratio of the expression of ESTs for a particular cluster to the expression of all ESTs in that category, according to parts per million).

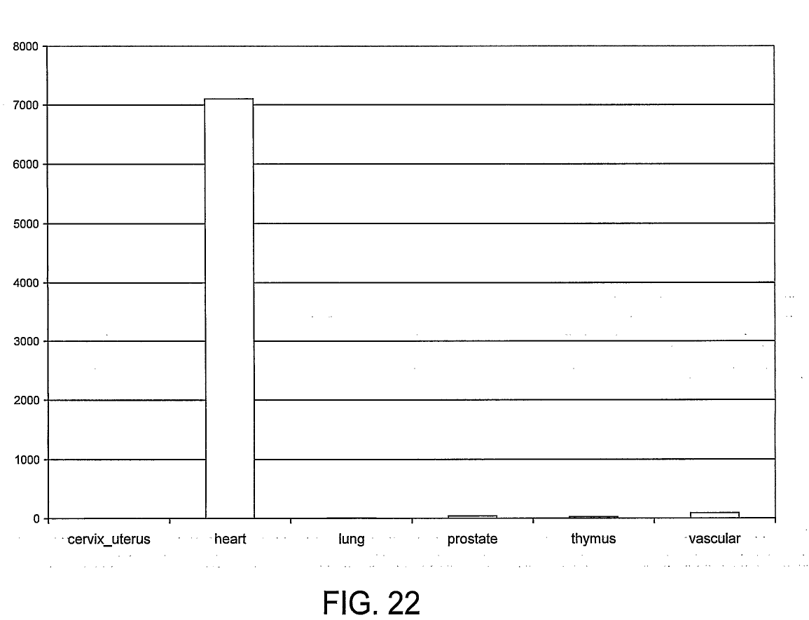

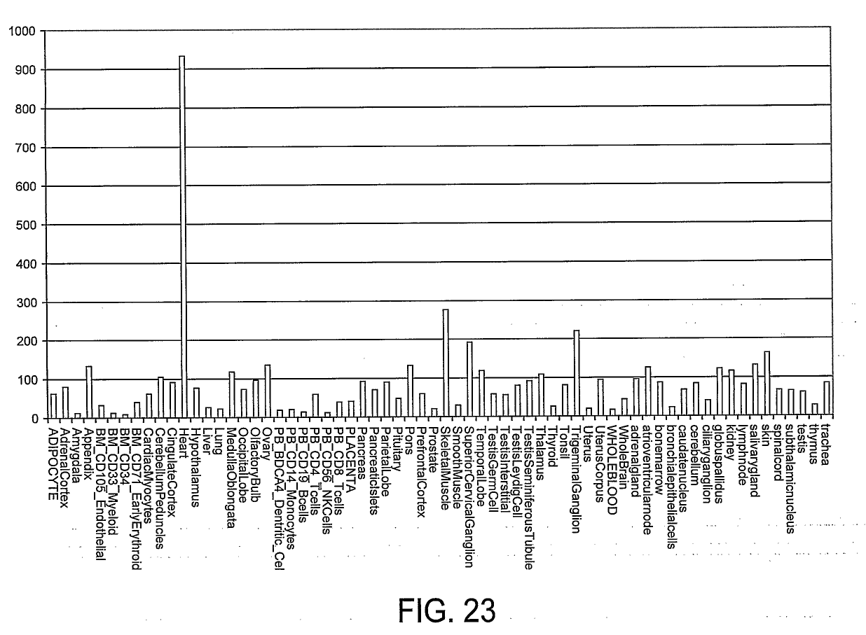

Overall, the following results were obtained as shown with regard to the histogram in FIG. 22, concerning the number of heart-specific clones in libraries/sequences; as well as with regard to the histogram in FIG. 23, concerning the actual expression of oligonucleotides in various tissues, including heart.

This cluster was found to be selectively expressed in heart for the following reasons: in a comparison of the ratio of expression of the cluster in heart specific ESTs to the overall expression of the cluster in non-heart ESTs, which was found to be 53.5; the ratio of expression of the cluster in heart specific ESTs to the overall expression of the cluster in muscle-specific ESTs which was found to be 3833.7; and fisher exact test P-values were computed both for library and weighted clone counts to check that the counts are statistically significant, and were found to be 1.40E-245.

One particularly important measure of specificity of expression of a cluster in heart tissue is the previously described comparison of the ratio of expression of the cluster in heart as opposed to muscle. This cluster was found to be specifically expressed in heart as opposed to non-heart ESTs as described above. However, many proteins have been shown to be generally expressed at a higher level in both heart and muscle, which is less desirable. For this cluster, as described above, the ratio of expression of the cluster in heart specific ESTs to the overall expression of the cluster in muscle-specific ESTs which was found to be 53.5, which clearly supports specific expression in heart tissue.

As noted above, cluster HUMCDDANF features 3 transcript(s), which were listed in Table 61 above. These transcript(s) encode for protein(s) which are variant(s) of protein Atrial natriuretic factor precursor (SEQ ID NO:164). A description of each variant protein according to the present invention is now provided.

Variant protein HUMCDDANF_PEA_{—}1_P6 (SEQ ID NO:165) according to the present invention has an amino acid sequence; it is encoded by transcript(s) HUMCDDANF_PEA_{—}1_T6 (SEQ ID NO:151). An alignment is given to the known protein (Atrial natriuretic factor precursor (SEQ ID NO:164)) at the end of the application. One or more alignments to one or more previously published protein sequences are given in the alignment table located on the attached CDROM. A brief description of the relationship of the variant protein according to the present invention to each such aligned protein is as follows:

Comparison report between HUMCDDANF_PEA_{—}1_P6 (SEQ ID NO:165) and ANF_HUMAN (SEQ ID NO:164):

1. An isolated chimeric polypeptide encoding for HUMCDDANF_PEA_{—}1_P6 (SEQ ID NO:165), comprising a first amino acid sequence being at least 90% homologous to MSSFSTTTVSFLLLLAFQLLGQTRANPMYNAVSNADLMDFKNLLDHLEEKMPLEDEVVPPQVLSE PNEEAGAALSPLPEVPPWTGEVSPAQRDGGALGRGPWDSSDRSALLKSKLRALLTAPRSLRRSSCF GGRMDRIGAQSGLGCNSFR corresponding to amino acids 1-150 of ANF_HUMAN (SEQ ID NO:164), which also corresponds to amino acids 1-150 of HUMCDDANF_PEA_{—}1_P6 (SEQ ID NO:165), and a second amino acid sequence being at least 70%, optionally at least 80%, preferably at least 85%, more preferably at least 90% and most preferably at least 95% homologous to a polypeptide having the sequence VRGTGDGNGMGWTLLGDTFSRKGTNAEAHSLSSFCPNTQSAPWVSGHAIYCP (SEQ ID NO: 642) corresponding to amino acids 151-202 of HUMCDDANF_PEA_{—}1_P6 (SEQ ID NO:165), wherein said first amino acid sequence and second amino acid sequence are contiguous and in a sequential order.

2. An isolated polypeptide encoding for a tail of HUMCDDANF_PEA_{—}1_P6 (SEQ ID NO:165), comprising a polypeptide being at least 70%, optionally at least about 80%, preferably at least about 85%, more preferably at least about 90% and most preferably at least about 95% homologous to the sequence VRGTGDGNGMGWTLLGDTFSRKGTNAEAHSLSSFCPNTQSAPWVSGHAIYCP (SEQ ID NO: 642) in HUMCDDANF_PEA_{—}1_P6 (SEQ ID NO:165).

The location of the variant protein was determined according to results from a number of different software programs and analyses, including analyses from SignalP and other specialized programs. The variant protein is believed to be located as follows with regard to the cell: secreted.

The variant protein has the following domains, as determined by using InterPro. The domains are described in Table 66:

TABLE 66
InterPro domain(s)

| | | | Position(s) |
|---|---|---|---|
| InterPro ID | Domain description | Analysis type | on protein |
| IPR000663 | Natriuretic peptide | FPrintScan | 127-136, 136-145 |
| IPR002407 | Natriuretic peptide, | FPrintScan | 109-127, 11-29, |
| | atrial type | | 128-150, 32-50, |
| | | | 51-69, 72-89, |
| | | | 92-108 |
| IPR000663 | Natriuretic peptide | HMMPfam | 43-146 |
| IPR000663 | Natriuretic peptide | HMMSmart | 123-146 |
| IPR000663 | Natriuretic peptide | ScanRegExp | 130-146 |
| IPR002407 | Natriuretic peptide, | BlastProDom | 121-149 |
| | atrial type | | |
| IPR000663 | Natriuretic peptide | BlastProDom | 150-150, 26-120 |

Variant protein HUMCDDANF_PEA_{—}1_P6 (SEQ ID NO:165) is encoded by the following transcript(s): HUMCDDANF_PEA_{—}1_T6 (SEQ ID NO:151). The coding portion of transcript HUMCDDANF_PEA_{—}1_T6 (SEQ ID NO:151) starts at position 104 and ends at position 709.

Variant protein HUMCDDANF_PEA_{—}1_P9 (SEQ ID NO:166) according to the present invention has an amino acid sequence; it is encoded by transcript(s) HUMCDDANF_PEA_{—}1_T7 (SEQ ID NO:152) and HUMCDDANF_PEA_{—}1_T8 (SEQ ID NO:153). An alignment is given to the known protein (Atrial natriuretic factor precursor (SEQ ID NO:164)) at the end of the application. One or more alignments to one or more previously published protein sequences are given in the alignment table located on the attached CDROM. A brief description of the relationship of the variant protein according to the present invention to each such aligned protein is as follows:

Comparison report between HUMCDDANF_PEA_{—}1_P9 (SEQ ID NO:166) and ANF_HUMAN (SEQ ID NO:164):

1. An isolated chimeric polypeptide encoding for HUMCDDANF_PEA_{—}1_P9 (SEQ ID NO:166), comprising a first amino acid sequence being at least 90% homologous to MSSFSTTTVSFLLLLAFQLLGQTRANPMYNAVSNADLMDFK corresponding to amino acids 1-41 of ANF_HUMAN (SEQ ID NO:164), which also corresponds to amino acids 1-41 of HUMCDDANF_PEA_{—}1_P9 (SEQ ID NO:166), and a second amino acid sequence being at least 70%, optionally at least 80%, preferably at least 85%, more preferably at least 90% and most preferably at least 95% homologous to a polypeptide having the sequence VGPGKRVQSGARGLSDAVLTPLDFLQVSEVYPFPCIFLF (SEQ ID NO: 643) corresponding to amino acids 42-80 of HUMCDDANF_PEA_{—}1_P9 (SEQ ID NO:166), wherein said first amino acid sequence and second amino acid sequence are contiguous and in a sequential order.

2. An isolated polypeptide encoding for a tail of HUMCDDANF_PEA_{—}1_P9 (SEQ ID NO:166), comprising a polypeptide being at least 70%, optionally at least about 80%, preferably at least about 85%, more preferably at least about 90% and most preferably at least about 95% homologous to the sequence VGPGKRVQSGARGLSDAVLTPLDFLQVSEVYPFPCIFLF (SEQ ID NO: 643) in HUMCDDANF_PEA_{—}1_P9 (SEQ ID NO:166).

The location of the variant protein was determined according to results from a number of different software programs and analyses, including analyses from SignalP and other specialized programs. The variant protein is believed to be located as follows with regard to the cell: secreted.

The variant protein has the following domains, as determined by using InterPro. The domains are described in Table 67:

TABLE 67
InterPro domain(s)

| | | Analysis | Position(s) |
|---|---|---|---|
| InterPro ID | Domain description | type | on protein |
| IPR002407 | Natriuretic peptide, atrial type | FPrintScan | 11-29, 32-50 |

Variant protein HUMCDDANF_PEA_{—}1_P9 (SEQ ID NO:166) is encoded by the following transcript(s): HUMCDDANF_PEA_{—}1_T7 (SEQ ID NO:152) and HUMCDDANF_PEA_{—}1_T8 (SEQ ID NO:153).

The coding portion of transcript HUMCDDANF_PEA_{—}1_T7 (SEQ ID NO:152) starts at position 104 and ends at position 343.

### Description for Cluster HSACMHCP

Cluster HSACMHCP features 7 transcript(s) and 61 segment(s) of interest, the names for which are given in Tables 68 and 69. The selected protein variants are given in table 70.

TABLE 68
Transcripts of interest
Transcript Name

HSACMHCP_PEA_1_T2 (SEQ ID NO: 167)
HSACMHCP_PEA_1_T3 (SEQ ID NO: 168)
HSACMHCP_PEA_1_T4 (SEQ ID NO: 169)
HSACMHCP_PEA_1_T6 (SEQ ID NO: 170)
HSACMHCP_PEA_1_T7 (SEQ ID NO: 171)
HSACMHCP_PEA_1_T13 (SEQ ID NO: 172)
HSACMHCP_PEA_1_T17 (SEQ ID NO: 173)

TABLE 69
Segments of interest

| Segment Name | Corresponding Transcript(s) |
|---|---|
| HSACMHCP_PEA_1_node_20 (SEQ ID NO: 174) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_22 (SEQ ID NO: 175) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_25 (SEQ ID NO: 176) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_43 (SEQ ID NO: 177) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_45 (SEQ ID NO: 178) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_46 (SEQ ID NO: 179) | HSACMHCP_PEA_1_T17 (SEQ ID |
| | NO: 173) |
| HSACMHCP_PEA_1_node_48 (SEQ ID NO: 180) | HSACMHCP_PEA_1_T13 (SEQ ID |
| | NO: 172) |
| HSACMHCP_PEA_1_node_49 (SEQ ID NO: 181) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_57 (SEQ ID NO: 182) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_59 (SEQ ID NO: 183) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_81 (SEQ ID NO: 189) | HSACMHCP_PEA_1_T3 (SEQ ID |
| | NO: 168) |
| HSACMHCP_PEA_1_node_87 (SEQ ID NO: 190) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_89 (SEQ ID NO: 191) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_96 (SEQ ID NO: 192) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_97 (SEQ ID NO: 193) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_100 (SEQ ID NO: 194) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_105 (SEQ ID NO: 195) | HSACMHCP_PEA_1_T7 (SEQ ID |
| | NO: 171) |
| HSACMHCP_PEA_1_node_106 (SEQ ID NO: 196) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_107 (SEQ ID NO: 197) | HSACMHCP_PEA_1_T2 (SEQ ID |
| | NO: 167), HSACMHCP_PEA_1_T6 (SEQ |
| | ID NO: 170) and HSACMHCP_PEA_1_T7 |
| | (SEQ ID NO: 171) |
| HSACMHCP_PEA_1_node_108 (SEQ ID NO: 198) | HSACMHCP_PEA_1_T2 (SEQ ID |
| | NO: 167) and HSACMHCP_PEA_1_T7 |
| | (SEQ ID NO: 171) |
| HSACMHCP_PEA_1_node_111 (SEQ ID NO: 199) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_113 (SEQ ID NO: 200) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_16 (SEQ ID NO: 201) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_18 (SEQ ID NO: 202) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_23 (SEQ ID NO: 203) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_27 (SEQ ID NO: 204) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_29 (SEQ ID NO: 205) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_31 (SEQ ID NO: 206) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_33 (SEQ ID NO: 207) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_35 (SEQ ID NO: 208) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_37 (SEQ ID NO: 209) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_39 (SEQ ID NO: 210) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_40 (SEQ ID NO: 211) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T17 (SEQ ID |
| | NO: 173) |
| HSACMHCP_PEA_1_node_51 (SEQ ID NO: 212) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_53 (SEQ ID NO: 213) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_55 (SEQ ID NO: 214) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_69 (SEQ ID NO: 215) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_72 (SEQ ID NO: 216) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_73 (SEQ ID NO: 217) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_74 (SEQ ID NO: 218) | HSACMHCP_PEA_1_T2 (SEQ TD |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_77 (SEQ ID NO: 219) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_78 (SEQ ID NO: 220) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_80 (SEQ ID NO: 221) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_82 (SEQ ID NO: 222) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_83 (SEQ ID NO: 223) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_84 (SEQ ID NO: 224) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_85 (SEQ ID NO: 225) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_90 (SEQ ID NO: 226) | HSACMHCP_PEA_1_T4 (SEQ ID |
| | NO: 169) |
| HSACMHCP_PEA_1_node_91 (SEQ ID NO: 227) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_92 (SEQ ID NO: 228) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_93 (SEQ ID NO: 229) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_95 (SEQ ID NO: 230) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_98 (SEQ ID NO: 231) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_103 (SEQ ID NO: 232) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_104 (SEQ ID NO: 233) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |
| HSACMHCP_PEA_1_node_109 (SEQ ID NO: 234) | HSACMHCP_PEA_1_T2 (SEQ ID |
| | NO: 167), HSACMHCP_PEA_1_T3 (SEQ |
| | ID NO: 168), HSACMHCP_PEA_1_T4 |
| | (SEQ ID NO: 169), |
| | HSACMHCP_PEA_1_T6 (SEQ ID |
| | NO: 170), HSACMHCP_PEA_1_T7 (SEQ |
| | ID NO: 171) and |
| | HSACMHCP_PEA_1_T13 (SEQ ID |
| | NO: 172) |

TABLE 70
Proteins of interest

| Protein Name | Corresponding Transcript(s) |
|---|---|
| HSACMHCP_PEA_1_P2 (SEQ ID NO: 239) | HSACMHCP_PEA_1_T2 (SEQ ID NO: 167); |
| | HSACMHCP_PEA_1_T6 (SEQ ID NO: 170) |
| HSACMHCP_PEA_1_P3 (SEQ ID NO: 240) | HSACMHCP_PEA_1_T3 (SEQ ID NO: 168) |
| HSACMHCP_PEA_1_P4 (SEQ ID NO: 241) | HSACMHCP_PEA_1_T4 (SEQ ID NO: 169) |
| HSACMHCP_PEA_1_P6 (SEQ ID NO: 242) | HSACMHCP_PEA_1_T7 (SEQ ID NO: 171) |
| HSACMHCP_PEA_1_P12 (SEQ ID NO: 243) | HSACMHCP_PEA_1_T13 (SEQ ID NO: 172) |
| HSACMHCP_PEA_1_P16 (SEQ ID NO: 244) | HSACMHCP_PEA_1_T17 (SEQ ID NO: 173) |

These sequences are variants of the known protein Myosin heavy chain (SEQ ID NO:235), cardiac muscle alpha isoform (SwissProt accession identifier MYH6_HUMAN (SEQ ID NO:235); known also according to the synonyms MyHC-alpha), referred to herein as the previously known protein.

Protein Myosin heavy chain (SEQ ID NO:235), cardiac muscle alpha isoform is known or believed to have the following function(s): Muscle contraction. Known polymorphisms for this sequence are as shown in Table 71.

TABLE 71
Amino acid mutations for Known Protein

| SNP position(s) on amino | |
|---|---|
| acid sequence | Comment |
| 88 | Q -> E |
| 574 | Q -> P |
| 608 | A -> G |
| 744 | T -> A |
| 790 | M -> I |
| 1014 | V -> A |
| 1021 | S -> T |
| 1101 | A -> V |
| 1290 | A -> S |
| 1373 | W -> C |
| 1533 | K -> N |
| 1540 | L -> M |
| 1577-1578 | KL -> NV |
| 1705-1706 | EQ -> DR |
| 1733 | E -> D |
| 1734 | A -> S |
| 1737 | T -> S |
| 1763 | D -> H |
| 1788 | M -> I |
| 1871 | D -> N |
| 1882 | R -> G |
| 1890 | Q -> R |
| 1933 | Missing |

Protein Myosin heavy chain (SEQ ID NO:235), cardiac muscle alpha isoform localization is believed to be Thick filaments of the myofibrils.

The following GO Annotation(s) apply to the previously known protein. The following annotation(s) were found: muscle contraction; striated muscle contraction; muscle development, which are annotation(s) related to Biological Process; microfilament motor; actin binding; calmodulin binding; ATP binding, which are annotation(s) related to Molecular Function; and muscle myosin; muscle thick filament; myosin, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of HSACMHCP may optionally have one or more of the following utilities, as described with regard to the Table 72 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

Table 72: Utilities for Variants of HSACMHCP, related to Myosin heavy chain (SEQ ID NO:235), cardiac muscle alpha isoform: MYH6_HUMAN (SEQ ID NO:235)

| | | |
|---|---|---|
| Genetic Marker | ASD—(Atrial septal defect) is one of the most | Nat Genet. 2005 Feb 27 |
| for ASD | common forms of congenital heart malformation. A new | |
| | locus linked with atrial septal defect on chromosome | |
| | 14q12 in a large family with dominantly inherited atrial | |
| | septal defect. The underlying mutation is a missense | |
| | substitution, I820N, in alpha-myosin heavy chain | |
| | (MYH6). | |
| Genetic | Hypertrophic Cardiomyopathy is an excessive | Circulation. 2002; |
| Marker for | thickening of the heart muscle accompanied with | 105: 446 |
| Hypertrophic | abnormal microscopic features. | |
| Cardiomyopathy | G→A transition (exon 20, nucleotide 2384) was | |
| | identified in the α-cardiac myosin heavy chain gene that | |
| | is substitutes glutamine for arginine at residue 795 | |
| | (Arg795Gln). | |
| | Substitution of a hydrophilic glutamine residue | |
| | for the appropriate basic arginine residue could disrupt | |
| | this αhelical domain and potentially disturb to the | |
| | interaction between the myosin heavy chain and the light | |
| | chain interactions. | |
| Predictive | Finding of no αMyHC protein in a setting of LV | Circulation Research. |
| Factor for the Risk of | dysfunction suggests that changes in cardiac MyHC | 2000; 86: 386 |
| Heart Failure (LV | expression can precede the development of overt | |
| dysfunction) | myocardial failure. | |
| Predictive Factor for | Myosin heavy chain (SEQ ID NO: 235) alpha gene | Mol Med. 2002 |
| disease progression in | expression can be selectively associated with alterations | Nov; 8(11): 750-60. |
| human DCM. | in dilated cardiomyopathy (DCM) phenotype. | |
| | Improvement in DCM phenotype was directly related to | |
| | a coordinate increase in alpha- and a decrease in beta- | |
| | myosin heavy chain mRNA expression. | |

According to other optional embodiments of the present invention, variants of this cluster according to the present invention (amino acid and/or nucleic acid sequences of HSACMHCP may optionally have one or more of the following utilities, some of which are related to utilities described above. It should be noted that these utilities are optionally and preferably suitable for human and non-human animals as subjects, except where otherwise noted.

A non-limiting example of such a utility is the detection, diagnosis and/or determination of myocardial failure. The method comprises detecting a HSACMHCP variant, for example a variant protein, protein fragment, peptide, polynucleotide, polynucleotide fragment and/or oligonucleotide as described herein, in a sample of a myocardial tissue from a ventricle of the heart, and optionally and preferably in a serum sample. The method further comprises quantitating the expression of alpha-myosin heavy chain (alpha-MHC) in the sample; and determining by statistical analysis if the expression in the sample is significantly different than the expression in normal subject.

Use of known protein alpha-MHC for diagnosing myocardial failure in human is described with regard to PCT Application No. WO9833942, hereby incorporated by reference as if fully set forth herein. The PCT application describes a method of diagnosing myocardial failure in a human comprising: obtaining a sample of myocardial tissue from a ventricle of the heart of the human; quantitating the expression of a-myosin heavy chain (a-MHC), b-myosin heavy chain (P-MHC), or both in the sample; and determining by statistical analysis if the expression of alpha-MHC, P-MHC, or both in the sample is significantly different than their expression in normal human ventricular myocardial tissue.

Another example of use of known alpha-MHC for the detection of heart disease, such as myocardial infraction and myocardial disease, is described with regard to EP Application No. EP0131834, and U.S. Pat. No. 4,943,427 hereby incorporated by reference as if fully set forth herein. These applications describe a method of diagnosis of atrial myocardial infarction, which possibly is combined with ventricular myocardial infarction preferably using detection of secretion of myosin heavy chain into the blood during myocardial infarction by performing an immunoassay with the monoclonal antibody specific for a-MHC. U.S. Pat. No. 4,943,427 describes a method for diagnosis of heart disease comprising a radiolabeled monoclonal antibody having specificity to cardiac myosin heavy chain or its active fragment, useful for example for topographic diagnosis of heart disease such as myocardial infarction and myocardial disease, preferably by imaging.

Another non-limiting example of possible a utility is the detection, diagnosis and/or determination of Hypertrophic Cardiomyopathy. The method comprises detecting a HSACMHCP variant, for example a variant protein, protein fragment, peptide, polynucleotide, polynucleotide fragment and/or oligonucleotide as described herein, for detection, diagnosis and/or determination of Hypertrophic cardiomyopathy. Use of known protein alpha-MHC for detecting the presence or absence of a mutation associated with hypertrophic cardiomyopathy (HC), including diagnosing familial HC (FHC) in a subject is described with regard to PCT Application No. WO9533856, hereby incorporated by reference as if fully set forth herein. The methods include providing DNA which encodes a sarcomeric thin filament protein (e.g., alpha-tropomyosin or cardiac troponin T) and detecting the presence or absence of a mutation in the amplified product which is associated with HC.

Another non-limiting example of possoble utility of HSACMHCP variant, for example a variant protein, protein fragment, peptide, polynucleotide, polynucleotide fragment and/or oligonucleotide as described herein, is for monitoring cell differentiation, useful for example in drug discovery or pharmacokinetic or pharmacological profiling. An example of method of use of known a-MHC for monitoring cell differentiation by the differentiation-dependent expression of a secreted reporter proteins, subject is described with regard to PCT Application No. WO05005662, hereby incorporated by reference as if fully set forth herein. The PCT describes a method of monitoring cell differentiation comprising: (a) culturing cells capable of differentiating into at least one particular cell type containing at least one recombinant nucleic acid molecule comprising a reporter gene encoding a product that is secreted upon cell differentiation, or maintaining a non-human animal comprising such cells, under conditions allowing differentiation of the cells; and (b) determining the amount or activity of the reporter gene product either within a body fluid of said transgenic non-human animal or the cell culture medium.

Another non-limiting example of possible a utility is the detection, diagnosis and/or determination of organ failure, more preferably heart failure. The method comprises detecting a HSACMHCP variant, for example a variant protein, protein fragment, peptide, polynucleotide, polynucleotide fragment and/or oligonucleotide as described herein, for detection, diagnosis and/or determination of organ failure, more preferably heart failure. Use of known protein alpha-MHC for predicting cardiac mortality rate, by contacting patient sample with antibody that binds to marker of cell injury, and to a second antibody that binds to a marker of organ adaptation, and determining the binding, is described with regard to PCT Application No. WO03020123, hereby incorporated by reference as if fully set forth herein.

Another non-limiting example of possible a utility is the detection, diagnosis and/or determination of systemic vasculature events. The method comprises detecting a HSACMHCP variant, for example a variant protein, protein fragment, peptide, polynucleotide, polynucleotide fragment and/or oligonucleotide as described herein, for detection, diagnosis and/or determination of systemic vasculature events, including but not limited to cardiovascular disease, stroke, pulmonary, renovascular, cerebrovascular, thrombotic or generalized arterial or venous condition or event including acute coronary syndrome such as but not limited to acute myocardial infarction, heart failure, atheromoma or a thrombotic condition. The identification of these parameters or more particularly a pattern of parameters enables the diagnosis of a condition or event or the determination of the risk of development of a condition or event associated to the systemic vasculature. It is also useful in determining the risk of a vascular disease including cardiovascular, stroke, pulmonary, renovascular, cerebrovascular, thrombotic or generalized arterial or venous conditions or events in a healthy subject or a subject entering into an exposure to risk such as surgery or chemotherapy. The present invention is useful inter alia for the identification and/or quantitation of biochemical markers of conditions or events in the systemic vasculature such as heart disease, heart disorders, infections of the heart, stroke and thrombosis as well as the determination of a risk of development of these conditions including the absence of disorders or absence of risk of the development of a disorder. An example of method of use of known a-MHC for diagnosis of systemic vasculature events is described with regard to PCT Application No. WO0223191, hereby incorporated by reference as if fully set forth herein. The PCT describes a diagnostic assay for systemic vasculature events comprises assaying an array of markers and correlating the results.

Another non-limiting example of a possible utility is the detection, diagnosis and/or determination of dissecting aortic aneurysm. The method comprises detecting a HSACMHCP variant, for example a variant protein, protein fragment, peptide, polynucleotide, polynucleotide fragment and/or oligonucleotide as described herein, for detection, diagnosis and/or determination of dissecting aortic aneurysm. An example of method of use of known alpha-MHC for diagnosis of dissecting aortic aneurysm is described with regard to EP Application No. EP0782863, hereby incorporated by reference as if fully set forth herein. The EP application describes diagnosing of dissecting aortic aneurysm by assaying the heavy chain of a smooth muscle myosin present in the blood of a patient by using an antibody against the heavy chain.

Another non-limiting example of possible a utility is the detection, diagnosis and/or determination of graft rejection. The method comprises detecting a HSACMHCP variant, for example a variant protein, protein fragment, peptide, polynucleotide, polynucleotide fragment and/or oligonucleotide as described herein, for detection, diagnosis and/or determination of graft rejection, more preferably cardiac transplant rejection. Allograft rejection is initiated by an immune response to donor major histocompatibility complex proteins. After allogeneic heart transplantation, de novo CD4+ T cell and B cell autoimmune responses to contractile proteins of cardiac muscle, e.g. cardiac myosin (CM), are elicited. The transplantation induced autoimmune response to cardiac myosin plays a significant role in cardiac transplant rejection. An example of a method of use of known a-MHC for diagnosis of graft rejection against a heart transplanted into a mammalian recipient, is described with regard to U.S. Pat. No. 6,358,751, hereby incorporated by reference as if fully set forth herein. The US patent describes a method comprising: detecting the presence of immune reactivity to autologous contractile proteins expressed in cardiac tissue and native to said mammalian recipient wherein said autologous contractile protein is a-myosin heavy chain; wherein the presence of said immune reactivity is indicative of rejection of said transplanted heart.

Cluster HSACMHCP belongs to a family of proteins which are known to have functions related to noninvasive infarct sizing, hypertrophic cardiomyopathy and muscle plasticity in response to various mechanical perturbations, including but not limited to, MYH13, MYH4, MYH7, MYH8, MYH3, MYH11, MYH14. These functions are described below; one or more variants of cluster HSACMHCP may optionally have one or more diagnostic utilities related to these functions.

Myosin light chains which may have value in noninvasive infarct sizing (Foreback C C.: Biochemical diagnosis of myocardial infarction. Henry Ford Hosp Med J. 1991; 39(3-4):159-64.); Acute myeloid leukaemia (AML) associated with the inversion chromosome 16 involving MYH11 (Kuss B J et al.: The biological significance of the multidrug resistance gene MRP in inversion 16 leukemias. Leuk Lymphoma. 1996 February; 20(5-6):357-64.); The first known cause of HCM (hypertrophic cardiomyopathy) was a point mutation in the cardiac beta-myosin heavy chain gene on chromosome 14 (Vosberg H P.: Identification of gene defects by linkage analysis: use in inherited cardiomyopathies. Eur Heart J. 1994 December; 15 Suppl D:20-3.); as the regulation of the myosin gene family is under the control of a complex set of processes including, but not limited to, activity, hormonal, and metabolic factors, this protein will serve as a cellular “marker” for studies of muscle plasticity in response to various mechanical perturbations in which the quantity and type of myosin isoform, along with other important cellular proteins, are altered in expression. (Baldwin K M, Haddad F.: Skeletal muscle plasticity: cellular and molecular responses to altered physical activity paradigms. Am J Phys Med Rehabil. 2002 November; 81 (11 Suppl):S40-51.). Smooth muscle myosin heavy chain (MYH11) is a specific marker of smooth muscle cells (Couffinhal T, et al.: Kinetics of adventitial repair in the rat carotid model. Coron Artery Dis. 2001 December; 12(8):635-48.); Studies using an assay developed against smooth muscle myosin heavy chain, a protein which is released from the aortic medial smooth muscle cells on insult to the aortic wall, showed promising results for use of this assay in the diagnosis of aortic dissection. (Suzuki T, Katoh H, Nagai R.: Biochemical diagnosis of aortic dissection: from bench to bedside. Jpn Heart J. 1999 September; 40(5):527-34.). Age-related sarcopenia is associated with Myosin Heavy chain proteins (Short K R, Nair K S.: Mechanisms of sarcopenia of aging. J Endocrinol Invest. 1999; 22(5 Suppl):95-105.). Hypertrophic cardiomyopathy is indicated by mutations in Myosin heavy chain (SEQ ID NO:235) beta (Seiler C.: Hypertrophic cardiomyopathy: spontaneous course Schweiz Med Wochenschr. 1995 Oct. 14; 125(41):1931-9.). Recent identification of mutations in the beta myosin heavy chain gene and genotype-phenotype correlation in HCM patients have shown that the beta myosin heavy chain mutations are also prognosticators in HCM families. (Marian A J.: Sudden cardiac death in patients with hypertrophic cardiomyopathy: from bench to bedside with an emphasis on genetic markers. Clin Cardiol. 1995 April; 18(4): 189-98.). All of these functions may optionally be diagnostic utilities of one or more HSACMHCP variants according to the present invention.

Table 73 below describes diagnostic utilities for the cluster HSACMHCP that were found through microarrays, including the statistical significance thereof and a reference. One or more HSACMHCP variants according to the present invention may optionally have one or more of these utilities.

TABLE 73

| | Statistical | |
|---|---|---|
| Diagnostic utility | significance | reference |
| Gene over expressed | 3.5E−4 | 1. Bhattacharjee A, Meyerson M |
| in lung cancer | | PNAS (2001) Classification of |
| Metastasis | | human lung carcinomas by |
| (vs. primary cancer). | | mRNA expression profiling |
| | | reveals distinct adenocarcinoma |
| | | subclasses |
| Gene over expressed | 6.1E−7 | 1. Nutt CL, Louis DN. Cancer |
| in Non-Classic Glioma | | Res (2003) Gene expression- |
| (vs. Classical). | | based classification of |
| | | malignant gliomas correlates |
| | | better with survival than |
| | | histological classification. |

Other non-limiting exemplary utilities for HSACMHCP variants according to the present invention are described in greater detail below and also with regard to the previous section on clinical utility.

The heart-selective diagnostic marker prediction engine provided the following results with regard to cluster HSACMHCP. Predictions were made for selective expression of transcripts of this contig in heart tissue, according to the previously described methods. The numbers on the y-axis of the first figure below refer to weighted expression of ESTs in each category, as “parts per million” (ratio of the expression of ESTs for a particular cluster to the expression of all ESTs in that category, according to parts per million).

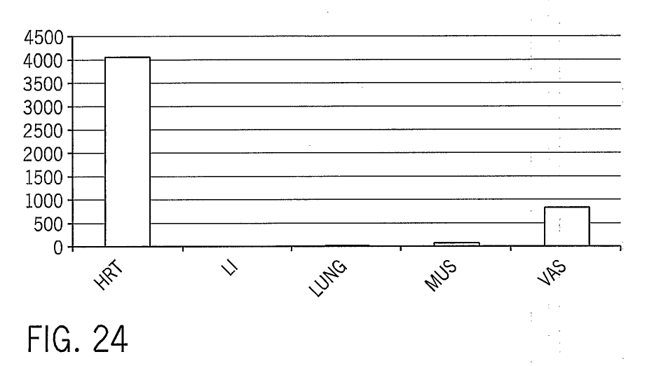

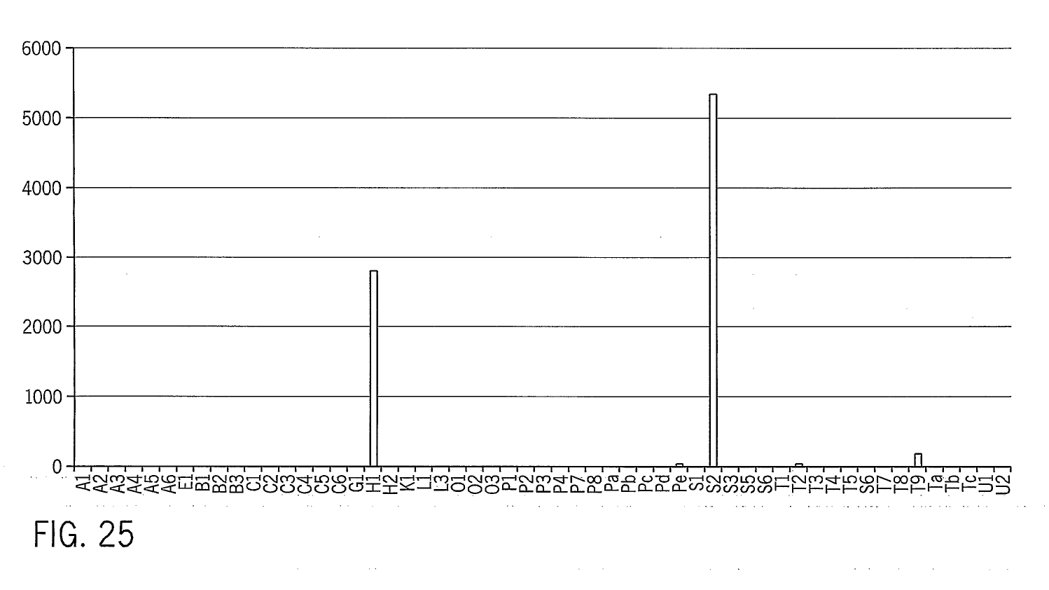

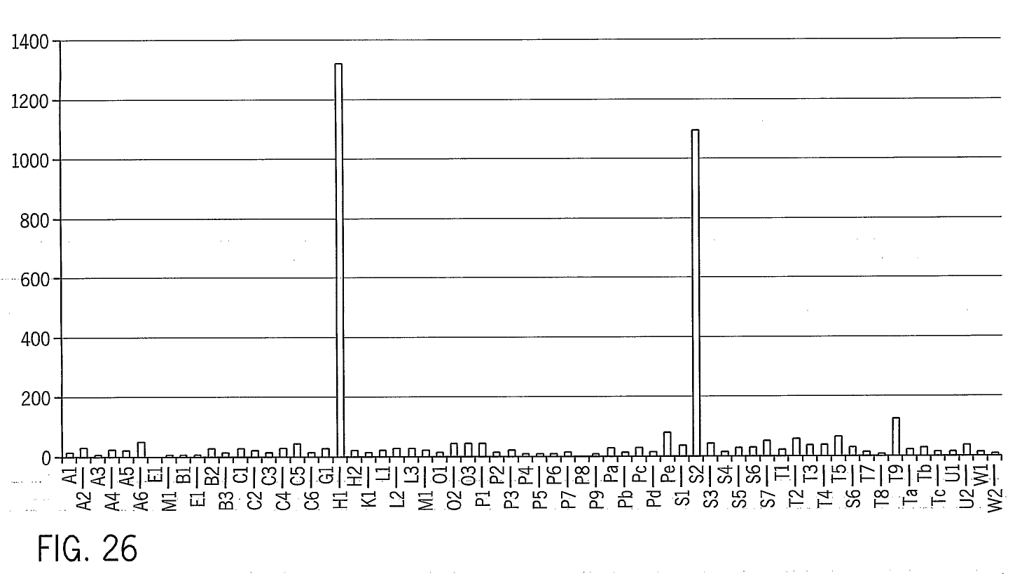

Overall, the following results were obtained as shown with regard to the histogram in FIG. 24, concerning the number of heart-specific clones in libraries/sequences; as well as with regard to the histogram in FIGS. 25-26, concerning the actual expression of oligonucleotides in various tissues, including heart.

This cluster was found to be selectively expressed in heart for the following reasons: in a comparison of the ratio of expression of the cluster in heart specific ESTs to the overall expression of the cluster in non-heart ESTs, which was found to be 24; the ratio of expression of the cluster in heart specific ESTs to the overall expression of the cluster in muscle-specific ESTs which was found to be 92.5; and fisher exact test P-values were computed both for library and weighted clone counts to check that the counts are statistically significant, and were found to be 3.20E-47.

One particularly important measure of specificity of expression of a cluster in heart tissue is the previously described comparison of the ratio of expression of the cluster in heart as opposed to muscle. This cluster was found to be specifically expressed in heart as opposed to non-heart ESTs as described above. However, many proteins have been shown to be generally expressed at a higher level in both heart and muscle, which is less desirable. For this cluster, as described above, the ratio of expression of the cluster in heart specific ESTs to the overall expression of the cluster in muscle-specific ESTs which was found to be 24, which clearly supports specific expression in heart tissue.

As noted above, cluster HSACMHCP features 7 transcript(s), which were listed in Table 68 above. These transcript(s) encode for protein(s) which are variant(s) of protein Myosin heavy chain (SEQ ID NO:235), cardiac muscle alpha isoform. A description of each variant protein according to the present invention is now provided.

Variant protein HSACMHCP_PEA_{—}1_P2 (SEQ ID NO:239) according to the present invention has an amino acid sequence; it is encoded by transcript(s) HSACMHCP_PEA_{—}1_T2 (SEQ ID NO:167). An alignment is given to the known protein (Myosin heavy chain (SEQ ID NO:235), cardiac muscle alpha isoform) at the end of the application. One or more alignments to one or more previously published protein sequences are given in the alignment table located on the attached CDROM. A brief description of the relationship of the variant protein according to the present invention to each such aligned protein is as follows:

Comparison report between HSACMHCP_PEA_{—}1_P2 (SEQ ID NO:239) and MYH6_HUMAN_V1 (SEQ ID NO:236):

1. An isolated chimeric polypeptide encoding for HSACMHCP_PEA_{—}1_P2 (SEQ ID NO:239), comprising a first amino acid sequence being at least 90% homologous to MTDAQMADFGAAAQYLRKSEKERLEAQTRPFDIRTECFVPDDKEEFVKAKILSREGGKVIAETEN GKTVTVKEDQVLQQNPPKFDKIEDMAMLTFLHEPAVLFNLKERYAAWMIYTYSGLFCVTVNPYK WLPVYNAEVVAAYRGKKRSEAPPHIFSISDNAYQYMLTDRENQSILITGESGAGKTVNTKRVIQYF ASIAAIGDRGKKDNANANKGTLEDQIIQANPALEAFGNAKTVRNDNSSRFGKFIRIHFGATGKLAS ADIETYLLEKSRVIFQLKAERNYHIFYQILSNKKPELLDMLLVTNNPYDYAFVSQGEVSVASIDDSE ELMATDSAFDVLGFTSEEKAGVYKLTGAIMHYGNMKFKQKQREEQAEPDGTEDADKSAYLMGL NSADLLKGLCHPRVKVGNEYVTKGQSVQQVYYSIGALAKAVYEKMFNWMVTRINATLETKQPR QYFIGVLDIAGFEIFDFNSFEQLCINFTNEKLQQFFNHHMFVLEQEEYKKEGIEWTFIDFGMDLQACI DLIEKPMGIMSILEEECMFPKATDMTFKAKLYDNHLGKSNNFQKPRNIKGKQEAHFSLIHYAGTVD YNILGWLEKNKDPLNETVVALYQKSSLKLMATLFSSYATADTGDSGKSKGGKKKGSSFQTVSAL HRENLNKLMTNLRTTHPHFVRCIIPNERKAPGVMDNPLVMHQLRCNGVLEGIRICRKGFPNRILYG DFRQRYRILNPVAIPEGQFIDSRKGTEKLLSSLDIDHNQYKFGHTKVFFKAGLLGLLEEMRDERLSR IITRMQAQARGQLMRIEFKKIVERRDALLVIQWNIRAFMGVKNWPWMKLYFKIKPLLKSAETEKE MATMKEEFGRIKETLEKSEARRKELEEKMVSLLQEKNDLQLQVQAEQDNLNDAEERCDQLIKNKI QLEAKVKEMNERLEDEEEMNAELTAKKRKLEDECSELKKDIDDLELTLAKVEKEKHATENKVKN LTEEMAGLDEIIAKLTKEKKALQEAHQQALDDLQVEEDKVNSLSKSKVKLEQQVDDLEGSLEQEK KVRMDLERAKRKLEGDLKLTQESIMDLENDKLQLEEKLKKKEFDINQQNSKIEDEQALALQLQKK LKENQARIEELEEELEAERTARAKVEKLRSDLSRELEEISERLEEAGGATSVQIEMNKKREAEFQK MRRDLEEATLQHEATAAALRKKHADSVAELGEQIDNLQRVKQKLEKEKSEFKLELDDVTSNMEQ IIKAKANLEKVSRTLEDQANEYRVKLEEAQRSLNDFTTQRAKLQTENGELARQLEEKEALISQLTR GKLSYTQQMEDLKRQLEEEGKAKNALAHALQSARHDCDLLREQYEEETEAKAELQRVLSKANSE VAQWRTKYETDAIQRTEELEEAKKKLAQRLQDAEEAVEAVNAKCSSLEKTKHRLQNEIEDLMVD VERSNAAAAALDKKQRNFDKILAEWKQKYEESQSELESSQKEARSLSTELFKLKNAYEESLEHLET FKRENKNLQEEISDLTEQLGEGGKNVHELEKVRKQLEVEKLELQSALEEAEASLEHEEGKILRAQL EFNQIKAEIERKLAEKDEEMEQAKRNHQRVVDSLQTSLDAETRSRNEVLRVKKKMEGDLNEMEIQ LSHANRMAAEAQKQVKSLQSLLKDTQIQLDDAVRANDDLKENIAIVERRNNLLQAELEELRAVVE QTERSRKLAEQELIETSERVQLLHSQNTSLINQKKKMESDLTQLQSEVEEAVQECRNAEEKAKKAI TDAAMMAEELKKEQDTSAHLERMKKNMEQTIKDLQHRLDEAEQIALKGGKKQLQKLEARVREL EGELEAEQKRNAESVKGMRKSERRIKELTYQ corresponding to amino acids 1-1855 of MYH6_HUMAN_V1 (SEQ ID NO:236), which also corresponds to amino acids 1-1855 of HSACMHCP_PEA_{—}1_P2 (SEQ ID NO:239), and a second amino acid sequence being at least 70%, optionally at least 80%, preferably at least 85%, more preferably at least 90% and most preferably at least 95% homologous to a polypeptide having the sequence VRRTPDTGSRCGSFFSGPTAPPSQGSSHLLLEMLLVDLTFFSRSAVSLT (SEQ ID NO: 644) corresponding to amino acids 1856-1904 of HSACMHCP_PEA_{—}1_P2 (SEQ ID NO:239), wherein said first amino acid sequence and second amino acid sequence are contiguous and in a sequential order.

2. An isolated polypeptide encoding for a tail of HSACMHCP_PEA_{—}1_P2 (SEQ ID NO:239), comprising a polypeptide being at least 70%, optionally at least about 80%, preferably at least about 85%, more preferably at least about 90% and most preferably at least about 95% homologous to the sequence VRRTPDTGSRCGSFFSGPTAPPSQGSSHLLLEMLLVDLTFFSRSAVSLT (SEQ ID NO: 644) in HSACMHCP_PEA_{—}1_P2 (SEQ ID NO:239).

It should be noted that the known protein sequence (MYH6_HUMAN (SEQ ID NO:235)) has one or more changes than the sequence named as being the amino acid sequence for MYH6_HUMAN_V1 (SEQ ID NO:236). These changes were previously known to occur and are listed in table 74 below.

TABLE 74
Changes to MYH6_HUMAN_V1 (SEQ ID NO: 236)

| SNP position(s) on amino | |
|---|---|
| acid sequence | Type of change |
| 89 | conflict |
| 1735 | conflict |

The location of the variant protein was determined according to results from a number of different software programs and analyses, including analyses from SignalP and other specialized programs. The variant protein is believed to be located as follows with regard to the cell: intracellularly.

The phosphorylation sites of variant protein HSACMHCP_PEA_{—}1_P2 (SEQ ID NO:239), as compared to the known protein Myosin heavy chain (SEQ ID NO:235), cardiac muscle alpha isoform, are described in Table 75 (given according to their position(s) on the amino acid sequence in the first column; the second column indicates whether the phosphorylation site is present in the variant protein; and the last column indicates whether the position is different on the variant protein).

TABLE 75
Phosphorylation site(s)

| Position(s) on known | Present in | Position in |
|---|---|---|
| amino acid sequence | variant protein? | variant protein? |
| 129 | Yes | 129 |

The variant protein has the following domains, as determined by using InterPro. The domains are described in Table 76:

TABLE 76
InterPro domain(s)

| InterPro ID | Domain description | Analysis type | Position(s) on protein |
|---|---|---|---|
| IPR001609 | Myosin head (motor domain) | FPrintScan | 115-134, 171-196, 226-253, |
| | | | 457-485, 511-539 |
| IPR001609 | Myosin head (motor domain) | HMMPfam | 87-768 |
| IPR002928 | Myosin tail | HMMPfam | 1070-1856 |
| IPR004009 | Myosin N-terminal SH3-like | HMMPfam | 34-77 |
| | domain | | |
| IPR001609 | Myosin head (motor domain) | HMMSmart | 79-781 |
| IPR001609 | Myosin head (motor domain) | BlastProDom | 168-253 |
| IPR000048 | IQ calmodulin-binding region | ProfileScan | 783-812 |

Variant protein HSACMHCP_PEA_{—}1_P2 (SEQ ID NO:239) is encoded by the following transcript(s): HSACMHCP_PEA_{—}1_T2 (SEQ ID NO:167). The coding portion of transcript HSACMHCP_PEA_{—}1_T2 (SEQ ID NO:167) starts at position 78 and ends at position 5789.

Variant protein HSACMHCP_PEA_{—}1_P3 (SEQ ID NO:240) according to the present invention has an amino acid sequence; it is encoded by transcript(s) HSACMHCP_PEA_{—}1_T3 (SEQ ID NO:168). An alignment is given to the known protein (Myosin heavy chain (SEQ ID NO:235), cardiac muscle alpha isoform) at the end of the application. One or more alignments to one or more previously published protein sequences are given in the alignment table located on the attached CDROM. A brief description of the relationship of the variant protein according to the present invention to each such aligned protein is as follows:

Comparison report between HSACMHCP_PEA_{—}1_P3 (SEQ ID NO:240) and MYH6_HUMAN_V2 (SEQ ID NO:237):

1. An isolated chimeric polypeptide encoding for HSACMHCP_PEA_{—}1_P3 (SEQ ID NO:240), comprising a first amino acid sequence being at least 90% homologous to MTDAQMADFGAAAQYLRKSEKERLEAQTRPFDIRTECFVPDDKEEFVKAKILSREGGKVIAETEN GKTVTVKEDQVLQQNPPKFDKIEDMAMLTFLHEPAVLFNLKERYAAWMIYTYSGLFCVTVNPYK WLPVYNAEVVAAYRGKKRSEAPPHIFSISDNAYQYMLTDRENQSILITGESGAGKTVNTKRVIQYF ASIAAIGDRGKKDNANANKGTLEDQIIQANPALEAFGNAKTVRNDNSSRFGKFIRIHFGATGKLAS ADIETYLLEKSRVIFQLKAERNYHIFYQILSNKKPELLDMLLVTNNPYDYAFVSQGEVSVASIDDSE ELMATDSAFDVLGFTSEEKAGVYKLTGAIMHYGNMKFKQKQREEQAEPDGTEDADKSAYLMGL NSADLLKGLCHPRVKVGNEYVTKGQSVQQVYYSIGALAKAVYEKMFNWMVTRINATLETKQPR QYFIGVLDIAGFEIFDFNSFEQLCINFTNEKLQQFFNHHMFVLEQEEYKKEGIEWTFIDFGMDLQACI DLIEKPMGIMSILEEECMFPKATDMTFKAKLYDNHLGKSNNFQKPRNIKGKQEAHFSLIHYAGTVD YNILGWLEKNKDPLNETVVALYQKSSLKLMATLFSSYATADTGDSGKSKGGKKKGSSFQTVSAL HRENLNKLMTNLRTTHPHFVRCIIPNERKAPGVMDNPLVMHQLRCNGVLEGIRICRKGFPNRILYG DFRQRYRILNPVAIPEGQFIDSRKGTEKLLSSLDIDHNQYKFGHTKVFFKAGLLGLLEEMRDERLSR IITRMQAQARGQLMRIEFKKIVERRDALLVIQWNIRAFMGVKNWPWMKLYFKIKPLLKSAETEKE MATMKEEFGRIKETLEKSEARRKELEEKMVSLLQEKNDLQLQVQAEQDNLNDAEERCDQLIKNKI QLEAKVKEMNERLEDEEEMNAELTAKKRKLEDECSELKKDIDDLELTLAKVEKEKHATENKVKN LTEEMAGLDEIIAKLTKEKKALQEAHQQALDDLQVEEDKVNSLSKSKVKLEQQVDDLEGSLEQEK KVRMDLERAKRKLEGDLKLTQESIMDLENDKLQLEEKLKKKEFDINQQNSKIEDEQALALQLQKK LKENQARIEELEEELEAERTARAKVEKLRSDLSRELEEISERLEEAGGATSVQIEMNKKREAEFQK MRRDLEEATLQHEATAAALRKKHADSVAELGEQIDNLQRVKQKLEKEKSEFKLELDDVTSNMEQ IIKAKANLEKVSRTLEDQANEYRVKLEEAQRSLNDFTTQRAKLQTENGELARQLEEKEALISQLTR GKLSYTQQMEDLKRQLEEEGK corresponding to amino acids 1-1326 of MYH6_HUMAN_V2 (SEQ ID NO:237), which also corresponds to amino acids 1-1326 of HSACMHCP_PEA_{—}1_P3 (SEQ ID NO:240), and a second amino acid sequence being at least 70%, optionally at least 80%, preferably at least 85%, more preferably at least 90% and most preferably at least 95% homologous to a polypeptide having the sequence VRPSGEGGQA (SEQ ID NO: 645) corresponding to amino acids 1327-1336 of HSACMHCP_PEA_{—}1_P3 (SEQ ID NO:240), wherein said first amino acid sequence and second amino acid sequence are contiguous and in a sequential order.

2. An isolated polypeptide encoding for a tail of HSACMHCP_PEA_{—}1_P3 (SEQ ID NO:240), comprising a polypeptide being at least 70%, optionally at least about 80%, preferably at least about 85%, more preferably at least about 90% and most preferably at least about 95% homologous to the sequence VRPSGEGGQA (SEQ ID NO: 645) in HSACMHCP_PEA_{—}1_P3 (SEQ ID NO:240).

It should be noted that the known protein sequence (MYH6_HUMAN (SEQ ID NO:235)) has one or more changes than the sequence named as being the amino acid sequence for MYH6_HUMAN_V2 (SEQ ID NO:237). These changes were previously known to occur and are listed in table 77 below.

TABLE 77
Changes to MYH6_HUMAN_V2 (SEQ ID NO: 237)

| SNP position(s) on amino | |
|---|---|
| acid sequence | Type of change |
| 89 | conflict |

The location of the variant protein was determined according to results from a number of different software programs and analyses, including analyses from SignalP and other specialized programs. The variant protein is believed to be located as follows with regard to the cell: intracellularly.

The phosphorylation sites of variant protein HSACMHCP_PEA_{—}1_P3 (SEQ ID NO:240), as compared to the known protein Myosin heavy chain (SEQ ID NO:235), cardiac muscle alpha isoform, are described in Table 78 (given according to their position(s) on the amino acid sequence in the first column; the second column indicates whether the phosphorylation site is present in the variant protein; and the last column indicates whether the position is different on the variant protein).

TABLE 78
Phosphorylation site(s)

| Position(s) on known | Present in | Position in |
|---|---|---|
| amino acid sequence | variant protein? | variant protein? |
| 129 | Yes | 129 |

The variant protein has the following domains, as determined by using InterPro. The domains are described in Table 79:

TABLE 79
InterPro domain(s)

| InterPro ID | Domain description | Analysis type | Position(s) on protein |
|---|---|---|---|
| IPR001609 | Myosin head (motor domain) | FPrintScan | 115-134, 171-196, 226-253, |
| | | | 457-485, 511-539 |
| IPR001609 | Myosin head (motor domain) | HMMPfam | 87-768 |
| IPR002928 | Myosin tail | HMMPfam | 1070-1328 |
| IPR004009 | Myosin N-terminal SH3-like | HMMPfam | 34-77 |
| | domain | | |
| IPR001609 | Myosin head (motor domain) | HMMSmart | 79-781 |
| IPR001609 | Myosin head (motor domain) | BlastProDom | 168-253 |
| IPR000048 | IQ calmodulin-binding region | ProfileScan | 783-812 |

Variant protein HSACMHCP_PEA_{—}1_P3 (SEQ ID NO:240) is encoded by the following transcript(s): HSACMHCP_PEA_{—}1_T3 (SEQ ID NO:168). The coding portion of transcript HSACMHCP_PEA_{—}1_T3 (SEQ ID NO:168) starts at position 78 and ends at position 4085.

Variant protein HSACMHCP_PEA_{—}1_P4 (SEQ ID NO:241) according to the present invention has an amino acid sequence; it is encoded by transcript(s) HSACMHCP_PEA_{—}1_T4 (SEQ ID NO:169). An alignment is given to the known protein (Myosin heavy chain (SEQ ID NO:235), cardiac muscle alpha isoform) at the end of the application. One or more alignments to one or more previously published protein sequences are given in the alignment table located on the attached CDROM. A brief description of the relationship of the variant protein according to the present invention to each such aligned protein is as follows:

Comparison report between HSACMHCP_PEA_{—}1_P4 (SEQ ID NO:241) and MYH6_HUMAN_V2 (SEQ ID NO:237):

1. An isolated chimeric polypeptide encoding for HSACMHCP_PEA_{—}1_P4 (SEQ ID NO:241), comprising a first amino acid sequence being at least 90% homologous to MTDAQMADFGAAAQYLRKSEKERLEAQTRPFDIRTECFVPDDKEEFVKAKILSREGGKVIAETEN GKTVTVKEDQVLQQNPPKFDKIEDMAMLTFLHEPAVLFNLKERYAAWMIYTYSGLFCVTVNPYK WLPVYNAEVVAAYRGKKRSEAPPHIFSISDNAYQYMLTDRENQSILITGESGAGKTVNTKRVIQYF ASIAAIGDRGKKDNANANKGTLEDQIIQANPALEAFGNAKTVRNDNSSRFGKFIRIHFGATGKLAS ADIETYLLEKSRVIFQLKAERNYHIFYQILSNKKPELLDMLLVTNNPYDYAFVSQGEVSVASIDDSE ELMATDSAFDVLGFTSEEKAGVYKLTGAIMHYGNMKFKQKQREEQAEPDGTEDADKSAYLMGL NSADLLKGLCHPRVKVGNEYVTKGQSVQQVYYSIGALAKAVYEKMFNWMVTRINATLETKQPR QYFIGVLDIAGFEIFDFNSFEQLCINFTNEKLQQFFNHHMFVLEQEEYKKEGIEWTFIDFGMDLQACI DLIEKPMGIMSILEEECMFPKATDMTFKAKLYDNHLGKSNNFQKPRNIKGKQEAHFSLIHYAGTVD YNILGWLEKNKDPLNETVVALYQKSSLKLMATLFSSYATADTGDSGKSKGGKKKGSSFQTVSAL HRENLNKLMTNLRTTHPHFVRCIIPNERKAPGVMDNPLVMHQLRCNGVLEGIRICRKGFPNRILYG DFRQRYRILNPVAIPEGQFIDSRKGTEKLLSSLDIDHNQYKFGHTKVFFKAGLLGLLEEMRDERLSR IITRMQAQARGQLMRIEFKKIVERRDALLVIQWNIRAFMGVKNWPWMKLYFKIKPLLKSAETEKE MATMKEEFGRIKETLEKSEARRKELEEKMVSLLQEKNDLQLQVQAEQDNLNDAEERCDQLIKNKI QLEAKVKEMNERLEDEEEMNAELTAKKRKLEDECSELKKDIDDLELTLAKVEKEKHATENKVKN LTEEMAGLDEIIAKLTKEKKALQEAHQQALDDLQVEEDKVNSLSKSKVKLEQQVDDLEGSLEQEK KVRMDLERAKRKLEGDLKLTQESIMDLENDKLQLEEKLKKKEFDINQQNSKIEDEQALALQLQKK LKENQARIEELEEELEAERTARAKVEKLRSDLSRELEEISERLEEAGGATSVQIEMNKKREAEFQK MRRDLEEATLQHEATAAALRKKHADSVAELGEQIDNLQRVKQKLEKEKSEFKLELDDVTSNMEQ IIKAKANLEKVSRTLEDQANEYRVKLEEAQRSLNDFTTQRAKLQTENGELARQLEEKEALISQLTR GKLSYTQQMEDLKRQLEEEGKAKNALAHALQSARHDCDLLREQYEEETEAKAELQRVLSKANSE VAQWRTKYETDAIQRTEELEEAKKKLAQRLQDAEEAVEAVNAKCSSLEKTKHRLQNEIEDLMVD VERSNAAAAALDKKQRNFDKILAEWKQKYEESQSELESSQKEARSLSTELFKLKNAYEESLEHLET FKRENKNLQ corresponding to amino acids 1-1508 of MYH6_HUMAN-V2 (SEQ ID NO:237), which also corresponds to amino acids 1-1508 of HSACMHCP_PEA_{—}1_P4 (SEQ ID NO:241), and a second amino acid sequence being at least 70%, optionally at least 80%, preferably at least 85%, more preferably at least 90% and most preferably at least 95% homologous to a polypeptide having the sequence GVLGVQEARDELVGGRAMQGQGEHRL (SEQ ID NO: 646) corresponding to amino acids 1509-1534 of HSACMHCP_PEA_{—}1_P4 (SEQ ID NO:241), wherein said first amino acid sequence and second amino acid sequence are contiguous and in a sequential order.

2. An isolated polypeptide encoding for a tail of HSACMHCP_PEA_{—}1_P4 (SEQ ID NO:241), comprising a polypeptide being at least 70%, optionally at least about 80%, preferably at least about 85%, more preferably at least about 90% and most preferably at least about 95% homologous to the sequence GVLGVQEARDELVGGRAMQGQGEHRL (SEQ ID NO: 646) in HSACMHCP_PEA_{—}1_P4 (SEQ ID NO:241).

It should be noted that the known protein sequence (MYH6_HUMAN (SEQ ID NO:235)) has one or more changes than the sequence named as being the amino acid sequence for MYH6_HUMAN_V2 (SEQ ID NO:237). These changes were previously known to occur and are listed in table 80 below.

TABLE 80
Changes to MYH6_HUMAN_V2 (SEQ ID NO: 237)

| SNP position(s) on amino | |
|---|---|
| acid sequence | Type of change |
| 89 | Conflict |

The location of the variant protein was determined according to results from a number of different software programs and analyses, including analyses from SignalP and other specialized programs. The variant protein is believed to be located as follows with regard to the cell: intracellularly.

The phosphorylation sites of variant protein HSACMHCP_PEA_{—}1_P4 (SEQ ID NO:241), as compared to the known protein Myosin heavy chain (SEQ ID NO:235), cardiac muscle alpha isoform, are described in Table 81 (given according to their position(s) on the amino acid sequence in the first column; the second column indicates whether the phosphorylation site is present in the variant protein; and the last column indicates whether the position is different on the variant protein).

TABLE 81
Phosphorylation site(s)

| Position(s) on known | Present in | Position in |
|---|---|---|
| amino acid sequence | variant protein? | variant protein? |
| 129 | yes | 129 |

The variant protein has the following domains, as determined by using InterPro. The domains are described in Table 82:

TABLE 82
InterPro domain(s)

| | Domain | | Position(s) |
|---|---|---|---|
| InterPro ID | description | Analysis type | on protein |
| IPR001609 | Myosin head | FPrintScan | 115-134, 171-196, |
| | (motor domain) | | 226-253, 457-485, |
| | | | 511-539 |
| IPR001609 | Myosin head | HMMPfam | 87-768 |
| | (motor domain) | | |
| IPR002928 | Myosin tail | HMMPfam | 1070-1508 |
| IPR004009 | Myosin N-terminal | HMMPfam | 34-77 |
| | SH3-like domain | | |
| IPR001609 | Myosin head | HMMSmart | 79-781 |
| | (motor domain) | | |
| IPR001609 | Myosin head | BlastProDom | 168-253 |
| | (motor domain) | | |
| IPR000048 | IQ calmodulin- | ProfileScan | 783-812 |
| | binding region | | |

Variant protein HSACMHCP_PEA_{—}1_P4 (SEQ ID NO:241) is encoded by the following transcript(s): HSACMHCP_PEA_{—}1_T4 (SEQ ID NO:169). The coding portion of transcript HSACMHCP_PEA_{—}1_T4 (SEQ ID NO:169) starts at position 78 and ends at position 4679.

Variant protein HSACMHCP_PEA_{—}1_P6 (SEQ ID NO:242) according to the present invention has an amino acid sequence; it is encoded by transcript(s) HSACMHCP_PEA_{—}1_T7 (SEQ ID NO:171). An alignment is given to the known protein (Myosin heavy chain (SEQ ID NO:235), cardiac muscle alpha isoform) at the end of the application. One or more alignments to one or more previously published protein sequences are given in the alignment table located on the attached CDROM. A brief description of the relationship of the variant protein according to the present invention to each such aligned protein is as follows:

Comparison report between HSACMHCP_PEA_{—}1_P6 (SEQ ID NO:242) and MYH6_HUMAN_V1 (SEQ ID NO:236):

1. An isolated chimeric polypeptide encoding for HSACMHCP_PEA_{—}1_P6 (SEQ ID NO:242), comprising a first amino acid sequence being at least 90% homologous to MTDAQMADFGAAAQYLRKSEKERLEAQTRPFDIRTECFVPDDKEEFVKAKILSREGGKVIAETEN GKTVTVKEDQVLQQNPPKFDKIEDMAMLTFLHEPAVLFNLKERYAAWMIYTYSGLFCVTVNPYK WLPVYNAEVVAAYRGKKRSEAPPHIFSISDNAYQYMLTDRENQSILITGESGAGKTVNTKRVIQYF ASIAAIGDRGKKDNANANKGTLEDQIIQANPALEAFGNAKTVRNDNSSRFGKFIRIHFGATGKLAS ADIETYLLEKSRVIFQLKAERNYHIFYQILSNKKPELLDMLLVTNNPYDYAFVSQGEVSVASIDDSE ELMATDSAFDVLGFTSEEKAGVYKLTGAIMHYGNMKFKQKQREEQAEPDGTEDADKSAYLMGL NSADLLKGLCHPRVKVGNEYVTKGQSVQQVYYSIGALAKAVYEKMFNWMVTRINATLETKQPR QYFIGVLDIAGFEIFDFNSFEQLCINFTNEKLQQFFNHHMFVLEQEEYKKEGIEWTFIDFGMDLQACI DLIEKPMGIMSILEEECMFPKATDMTFKAKLYDNHLGKSNNFQKPRNIKGKQEAHFSLIHYAGTVD YNILGWLEKNKDPLNETVVALYQKSSLKLMATLFSSYATADTGDSGKSKGGKKKGSSFQTVSAL HRENLNKLMTNLRTTHPHFVRCIIPNERKAPGVMDNPLVMHQLRCNGVLEGIRICRKGFPNRILYG DFRQRYRILNPVAIPEGQFIDSRKGTEKLLSSLDIDHNQYKFGHTKVFFKAGLLGLLEEMRDERLSR IITRMQAQARGQLMRIEFKKIVERRDALLVIQWNIRAFMGVKNWPWMKLYFKIKPLLKSAETEKE MATMKEEFGRIKETLEKSEARRKELEEKMVSLLQEKNDLQLQVQAEQDNLNDAEERCDQLIKNKI QLEAKVKEMNERLEDEEEMNAELTAKKRKLEDECSELKKDIDDLELTLAKVEKEKHATENKVKN LTEEMAGLDEIIAKLTKEKKALQEAHQQALDDLQVEEDKVNSLSKSKVKLEQQVDDLEGSLEQEK KVRMDLERAKRKLEGDLKLTQESIMDLENDKLQLEEKLKKKEFDINQQNSKIEDEQALALQLQKK LKENQARIEELEEELEAERTARAKVEKLRSDLSRELEEISERLEEAGGATSVQIEMNKKREAEFQK MRRDLEEATLQHEATAAALRKKHADSVAELGEQIDNLQRVKQKLEKEKSEFKLELDDVTSNMEQ IIKAKANLEKVSRTLEDQANEYRVKLEEAQRSLNDFTTQRAKLQTENGELARQLEEKEALISQLTR GKLSYTQQMEDLKRQLEEEGKAKNALAHALQSARHDCDLLREQYEEETEAKAELQRVLSKANSE VAQWRTKYETDAIQRTEELEEAKKKLAQRLQDAEEAVEAVNAKCSSLEKTKHRLQNEIEDLMVD VERSNAAAAALDKKQRNFDKILAEWKQKYEESQSELESSQKEARSLSTELFKLKNAYEESLEHLET FKRENKNLQEEISDLTEQLGEGGKNVHELEKVRKQLEVEKLELQSALEEAEASLEHEEGKILRAQL EFNQIKAEIERKLAEKDEEMEQAKRNHQRVVDSLQTSLDAETRSRNEVLRVKKKMEGDLNEMEIQ LSHANRMAAEAQKQVKSLQSLLKDTQIQLDDAVRANDDLKENIAIVERRNNLLQAELEELRAVVE QTERSRKLAEQELIETSERVQLLHSQNTSLINQKKKMESDLTQLQSEVEEAVQECRNAEEKAKKAI TD corresponding to amino acids 1-1763 of MYH6_HUMAN_V1 (SEQ ID NO:236), which also corresponds to amino acids 1-1763 of HSACMHCP_PEA_{—}1_P6 (SEQ ID NO:242), and a second amino acid sequence being at least 70%, optionally at least 80%, preferably at least 85%, more preferably at least 90% and most preferably at least 95% homologous to a polypeptide having the sequence VSDRPPSASPKDRNKALGPGQATVL (SEQ ID NO: 647) corresponding to amino acids 1764-1788 of HSACMHCP_PEA_{—}1_P6 (SEQ ID NO:242), wherein said first amino acid sequence and second amino acid sequence are contiguous and in a sequential order.

2. An isolated polypeptide encoding for a tail of HSACMHCP_PEA_{—}1_P6 (SEQ ID NO:242), comprising a polypeptide being at least 70%, optionally at least about 80%, preferably at least about 85%, more preferably at least about 90% and most preferably at least about 95% homologous to the sequence VSDRPPSASPKDRNKALGPGQATVL (SEQ ID NO: 647) in HSACMHCP_PEA_{—}1_P6 (SEQ ID NO:242).

It should be noted that the known protein sequence (MYH6_HUMAN (SEQ ID NO:235)) has one or more changes than the sequence named as being the amino acid sequence for MYH6_HUMAN_V1 (SEQ ID NO:236). These changes were previously known to occur and are listed the 83 table below.

TABLE 83
Changes to MYH6_HUMAN_V1 (SEQ ID NO: 236)

| SNP position(s) on amino | |
|---|---|
| acid sequence | Type of change |
| 89 | Conflict |
| 1735 | Conflict |

The location of the variant protein was determined according to results from a number of different software programs and analyses, including analyses from SignalP and other specialized programs. The variant protein is believed to be located as follows with regard to the cell: intracellularly.

The phosphorylation sites of variant protein HSACMHCP_PEA_{—}1_P6 (SEQ ID NO:242), as compared to the known protein Myosin heavy chain (SEQ ID NO:235), cardiac muscle alpha isoform, are described in Table 84 (given according to their position(s) on the amino acid sequence in the first column; the second column indicates whether the phosphorylation site is present in the variant protein; and the last column indicates whether the position is different on the variant protein).

TABLE 84
Phosphorylation site(s)

| Position(s) on known | Present in | Position in |
|---|---|---|
| amino acid sequence | variant protein? | variant protein? |
| 129 | Yes | 129 |

The variant protein has the following domains, as determined by using InterPro. The domains are described in Table 85:

TABLE 85
InterPro domain(s)

| InterPro ID | Domain description | Analysis type | Position(s) on protein |
|---|---|---|---|
| IPR001609 | Myosin head (motor domain) | FPrintScan | 115-134, 171-196, 226-253, |
| | | | 457-485, 511-539 |
| IPR001609 | Myosin head (motor domain) | HMMPfam | 87-768 |
| IPR002928 | Myosin tail | HMMPfam | 1070-1764 |
| IPR004009 | Myosin N-terminal SH3-like | HMMPfam | 34-77 |
| | domain | | |
| IPR001609 | Myosin head (notor domain) | HMMSmart | 79-781 |
| IPR001609 | Myosin head (motor domain) | BlastProDom | 168-253 |
| IPR000048 | IQ calmodulin-binding region | ProfileScan | 782-812 |

Variant protein HSACMHCP_PEA_{—}1_P6 (SEQ ID NO:242) is encoded by the following transcript(s): HSACMHCP_PEA_{—}1_T7 (SEQ ID NO:171). The coding portion of transcript HSACMHCP_PEA_{—}1_T7 (SEQ ID NO:171) starts at position 78 and ends at position 5441.

Variant protein HSACMHCP_PEA_{—}1_P12 (SEQ ID NO:243) according to the present invention has an amino acid sequence; it is encoded by transcript(s) HSACMHCP_PEA_{—}1_T13 (SEQ ID NO:172). An alignment is given to the known protein (Myosin heavy chain (SEQ ID NO:235), cardiac muscle alpha isoform) at the end of the application. One or more alignments to one or more previously published protein sequences are given in the alignment table located on the attached CDROM. A brief description of the relationship of the variant protein according to the present invention to each such aligned protein is as follows:

Comparison report between HSACMHCP_PEA_{—}1_P12 (SEQ ID NO:243) and MYH6_HUMAN_V3 (SEQ ID NO:238):

1. An isolated chimeric polypeptide encoding for HSACMHCP_PEA_{—}1_P12 (SEQ ID NO:243), comprising a first amino acid sequence being at least 70%, optionally at least 80%, preferably at least 85%, more preferably at least 90% and most preferably at least 95% homologous to a polypeptide having the sequence MGLWKPGSVLSDSLFASSPCPQ (SEQ ID NO: 648) corresponding to amino acids 1-22 of HSACMHCP_PEA_{—}1_P12 (SEQ ID NO:243), and a second amino acid sequence being at least 90% homologous to PMGIMSILEEECMFPKATDMTFKAKLYDNHLGKSNNFQKPRNIKGKQEAHFSLIHYAGTVDYNIL GWLEKNKDPLNETVVALYQKSSLKLMATLFSSYATADTGDSGKSKGGKKKGSSFQTVSALHREN LNKLMTNLRTTHPHFVRCIIPNERKAPGVMDNPLVMHQLRCNGVLEGIRICRKGFPNRILYGDFRQ RYRILNPVAIPEGQFIDSRKGTEKLLSSLDIDHNQYKFGHTKVFFKAGLLGLLEEMRDERLSRIITRM QAQARGQLMRIEFKKIVERRDALLVIQWNIRAFMGVKNWPWMKLYFKIKPLLKSAETEKEMATM KEEFGRIKETLEKSEARRKELEEKMVSLLQEKNDLQLQVQAEQDNLNDAEERCDQLIKNKIQLEA KVKEMNERLEDEEEMNAELTAKKRKLEDECSELKKDIDDLELTLAKVEKEKHATENKVKNLTEE MAGLDEIIAKLTKEKKALQEAHQQALDDLQVEEDKVNSLSKSKVKLEQQVDDLEGSLEQEKKVR MDLERAKRKLEGDLKLTQESIMDLENDKLQLEEKLKKKEFDINQQNSKIEDEQALALQLQKKLKE NQARIEELEEELEAERTARAKVEKLRSDLSRELEEISERLEEAGGATSVQIEMNKKREAEFQKMRR DLEEATLQHEATAAALRKKHADSVAELGEQIDNLQRVKQKLEKEKSEFKLELDDVTSNMEQIIKA KANLEKVSRTLEDQANEYRVKLEEAQRSLNDFTTQRAKLQTENGELARQLEEKEALISQLTRGKL SYTQQMEDLKRQLEEEGKAKNALAHALQSARHDCDLLREQYEEETEAKAELQRVLSKANSEVAQ WRTKYETDAIQRTEELEEAKKKLAQRLQDAEEAVEAVNAKCSSLEKTKHRLQNEIEDLMVDVER SNAAAAALDKKQRNFDKILAEWKQKYEESQSELESSQKEARSLSTELFKLKNAYEESLEHLETFKR ENKNLQEEISDLTEQLGEGGKNVHELEKVRKQLEVEKLELQSALEEAEASLEHEEGKILRAQLEFN QIKAEIERKLAEKDEEMEQAKRNHQRVVDSLQTSLDAETRSRNEVLRVKKKMEGDLNEMEIQLSH ANRMAAEAQKQVKSLQSLLKDTQIQLDDAVRANDDLKENIAIVERRNNLLQAELEELRAVVEQTE RSRKLAEQELIETSERVQLLHSQNTSLINQKKKMESDLTQLQSEVEEAVQECRNAEEKAKKAITDA AMMAEELKKEQDTSAHLERMKKNMEQTIKDLQHRLDEAEQIALKGGKKQLQKLEARVRELEGE LEAEQKRNAESVKGMRKSERRIKELTYQTEEDKKNLLRLQDLVDKLQLKVKAYKRQAEEAEEQA NTNLSKFRKVQHELDEAEERADIAESQVNKLRAKSRDIGAKQKMHDEE corresponding to amino acids 528-1939 of MYH6_HUMAN_V3 (SEQ ID NO:238), which also corresponds to amino acids 23-1434 of HSACMHCP_PEA_{—}1_P12 (SEQ ID NO:243), wherein said first amino acid sequence and second amino acid sequence are contiguous and in a sequential order.

2. An isolated polypeptide encoding for a head of HSACMHCP_PEA_{—}1_P12 (SEQ ID NO:243), comprising a polypeptide being at least 70%, optionally at least about 80%, preferably at least about 85%, more preferably at least about 90% and most preferably at least about 95% homologous to the sequence MGLWKPGSVLSDSLFASSPCPQ (SEQ ID NO: 648) of HSACMHCP_PEA_{—}1_P12 (SEQ ID NO:243).

It should be noted that the known protein sequence (MYH6_HUMAN (SEQ ID NO:235)) has one or more changes than the sequence named as being the amino acid sequence for MYH6_HUMAN_V3 (SEQ ID NO:238). These changes were previously known to occur and are listed in table 86 below.

TABLE 86
Changes to MYH6_HUMAN_V3 (SEQ ID NO: 238)

| SNP position(s) on amino | |
|---|---|
| acid sequence | Type of change |
| 1735 | Conflict |

The location of the variant protein was determined according to results from a number of different software programs and analyses, including analyses from SignalP and other specialized programs. The variant protein is believed to be located as follows with regard to the cell: intracellularly.

The phosphorylation sites of variant protein HSACMHCP_PEA_{—}1_P12 (SEQ ID NO:243), as compared to the known protein Myosin heavy chain (SEQ ID NO:235), cardiac muscle alpha isoform, are described in Table 87 (given according to their position(s) on the amino acid sequence in the first column; the second column indicates whether the phosphorylation site is present in the variant protein; and the last column indicates whether the position is different on the variant protein).

TABLE 87
Phosphorylation site(s)

| Position(s) on known | Present in | Position in |
|---|---|---|
| amino acid sequence | variant protein? | variant protein? |
| 129 | No | |

The variant protein has the following domains, as determined by using InterPro. The domains are described in Table 88:

TABLE 88
InterPro domain(s)

| | Domain | | Position(s) |
|---|---|---|---|
| InterPro ID | description | Analysis type | on protein |
| IPR000533 | Tropomyosin | FPrintScan | 1308-1325, 1337-1357, |
| | | | 1369-1397, 1399-1422 |
| IPR001609 | Myosin head | HMMPfam | 22-263 |
| | (motor domain) | | |
| IPR002928 | Myosin tail | HMMPfam | 565-1424 |
| IPR001609 | Myosin head | HMMSmart | 1-276 |
| | (motor domain) | | |
| IPR000048 | IQ calmodulin- | ProfileScan | 278-307 |
| | binding region | | |

Variant protein HSACMHCP_PEA_{—}1_P12 (SEQ ID NO:243) is encoded by the following transcript(s): HSACMHCP_PEA_{—}1_T13 (SEQ ID NO:172). The coding portion of transcript HSACMHCP_PEA_{—}1_T13 (SEQ ID NO:172) starts at position 67 and ends at position 4368.

Variant protein HSACMHCP_PEA_{—}1_P16 (SEQ ID NO:244) according to the present invention has an amino acid sequence; it is encoded by transcript(s) HSACMHCP_PEA_{—}1_T17 (SEQ ID NO:173). An alignment is given to the known protein (Myosin heavy chain (SEQ ID NO:235), cardiac muscle alpha isoform) at the end of the application. One or more alignments to one or more previously published protein sequences are in the alignment table located on the attached CDROM. A brief description of the relationship of the variant protein according to the present invention to each such aligned protein is as follows:

Comparison report between HSACMHCP_PEA_{—}1_P16 (SEQ ID NO:244) and MYH6_HUMAN_V2 (SEQ ID NO:237):

1. An isolated chimeric polypeptide encoding for HSACMHCP_PEA_{—}1_P16 (SEQ ID NO:244), comprising a first amino acid sequence being at least 90% homologous to MTDAQMADFGAAAQYLRKSEKERLEAQTRPFDIRTECFVPDDKEEFVKAKILSREGGKVIAETEN GKTVTVKEDQVLQQNPPKFDKIEDMAMLTFLHEPAVLFNLKERYAAWMIYTYSGLFCVTVNPYK WLPVYNAEVVAAYRGKKRSEAPPHIFSISDNAYQYMLTDRENQSILITGESGAGKTVNTKRVIQYF ASIAAIGDRGKKDNANANKGTLEDQIIQANPALEAFGNAKTVRNDNSSRFGKFIRIHFGATGKLAS ADIETYLLEKSRVIFQLKAERNYHIFYQILSNKKPELLDMLLVTNNPYDYAFVSQGEVSVASIDDSE ELMATDSAFDVLGFTSEEKAGVYKLTGAIMHYGNMKFKQKQREEQAEPDGTEDADKSAYLMGL NSADLLKGLCHPRVKVGNEYVTKGQSVQQVYYSIGALAKAVYEKMFNWMVTRINATLETKQPR QYFIGVLDIAGFEIFDFNSFEQLCINFTNEKLQQFFNHHMFVLEQEEYKKEGIEWTFIDFGMDLQACI DLIEK corresponding to amino acids 1-527 of MYH6_HUMAN_V2 (SEQ ID NO:237), which also corresponds to amino acids 1-527 of HSACMHCP_PEA_{—}1_P16 (SEQ ID NO:244), and a second amino acid sequence being at least 70%, optionally at least 80%, preferably at least 85%, more preferably at least 90% and most preferably at least 95% homologous to a polypeptide having the sequence VPPWPHHLCPLLCHPDKVVAESLLHPRN (SEQ ID NO: 649) corresponding to amino acids 528-555 of HSACMHCP_PEA_{—}1_P16 (SEQ ID NO:244), wherein said first amino acid sequence and second amino acid sequence are contiguous and in a sequential order.

2. An isolated polypeptide encoding for a tail of HSACMHCP_PEA_{—}1_P16 (SEQ ID NO:244), comprising a polypeptide being at least 70%, optionally at least about 80%, preferably at least about 85%, more preferably at least about 90% and most preferably at least about 95% homologous to the sequence VPPWPHHLCPLLCHPDKVVAESLLHPRN (SEQ ID NO: 649) in HSACMHCP_PEA_{—}1_P16 (SEQ ID NO:244).

It should be noted that the known protein sequence (MYH6_HUMAN (SEQ ID NO:235)) has one or more changes than the sequence named as being the amino acid sequence for MYH6_HUMAN_V2 (SEQ ID NO:237). These changes were previously known to occur and are listed in table 89 below.

TABLE 89
Changes to MYH6_HUMAN_V2 (SEQ ID NO: 237)

| SNP position(s) on amino | |
|---|---|
| acid sequence | Type of change |
| 89 | conflict |

The location of the variant protein was determined according to results from a number of different software programs and analyses, including analyses from SignalP and other specialized programs. The variant protein is believed to be located as follows with regard to the cell: intracellularly.

The phosphorylation sites of variant protein HSACMHCP_PEA_{—}1_P16 (SEQ ID NO:244), as compared to the known protein Myosin heavy chain (SEQ ID NO:235), cardiac muscle alpha isoform, are described in Table 90 (given according to their position(s) on the amino acid sequence in the first column; the second column indicates whether the phosphorylation site is present in the variant protein; and the last column indicates whether the position is different on the variant protein).

TABLE 90
Phosphorylation site(s)

| Position(s) on known | Present in | Position in |
|---|---|---|
| amino acid sequence | variant protein? | variant protein? |
| 129 | yes | 129 |

The variant protein has the following domains, as determined by using InterPro. The domains are described in Table 91:

TABLE 91
InterPro domain(s)

| | Domain | | Position(s) |
|---|---|---|---|
| InterPro ID | description | Analysis type | on protein |
| IPR001609 | Myosin head | FPrintScan | 115-134, 171-196, |
| | (motor domain) | | 226-253, 457-485, |
| | | | 511-539 |
| IPR001609 | Myosin head | HMMPfam | 87-530 |
| | (motor domain) | | |
| IPR004009 | Myosin N-terminal | HMMPfam | 34-77 |
| | SH3-like domain | | |
| IPR001609 | Myosin head | HMMSmart | 79-554 |
| | (motor domain) | | |
| IPR001609 | Myosin head | BlastProDom | 168-253 |
| | (motor domain) | | |

Variant protein HSACMHCP_PEA_{—}1_P16 (SEQ ID NO:244) is encoded by the following transcript(s): HSACMHCP_PEA_{—}1_T17 (SEQ ID NO:173). The coding portion of transcript HSACMHCP_PEA_{—}1_T17 (SEQ ID NO:173) starts at position 78 and ends at position 1742.

Table 92 below describes the starting and ending position of HSACMHCP_PEA_{—}1_node_{—}46 (SEQ ID NO:179) on the relevant transcript. Experimental results for this segment are described below.

TABLE 92
Segment location on transcripts

| | Segment | Segment |
|---|---|---|
| Transcript name | starting position | ending position |
| HSACMHCP_PEA_1_T17 | 1659 | 2477 |
| (SEQ ID NO: 173) | | |

Table 93 below describes the starting and ending position of HSACMHCP_PEA_{—}1_node_{—}106 (SEQ ID NO:196) on each of the relevant transcripts. Experimental results for this segment are described below.

TABLE 93
Segment location on transcripts

| | Segment | Segment |
|---|---|---|
| Transcript name | starting position | ending position |
| HSACMHCP_PEA_1_T2 | 5367 | 5642 |
| (SEQ ID NO: 167) | | |
| HSACMHCP_PEA_1_T3 | 5704 | 5979 |
| (SEQ ID NO: 168) | | |
| HSACMHCP_PEA_1_T4 | 5471 | 5746 |
| (SEQ ID NO: 169) | | |
| HSACMHCP_PEA_1_T6 | 5367 | 5642 |
| (SEQ ID NO: 170) | | |
| HSACMHCP_PEA_1_T7 | 5565 | 5840 |
| (SEQ ID NO: 171) | | |
| HSACMHCP_PEA_1_T13 | 3841 | 4116 |
| (SEQ ID NO: 172) | | |

Expression of Homo sapiens myosin, heavy polypeptide 6, cardiac muscle, alpha (cardiomyopathy, hypertrophic 1) (MYH6) HSACMHCP transcripts which are detectable by amplicon as depicted in sequence name HSACMHCP seg106 (SEQ ID NO: 247) specifically in heart tissue:

Expression of Homo sapiens myosin, heavy polypeptide 6, cardiac muscle, alpha (cardiomyopathy, hypertrophic 1) (MYH6) transcripts detectable by or according to seg106—HSACMHCP seg106 (SEQ ID NO: 247) amplicon and primers HSACMHCP seg106F (SEQ ID NO: 245) and HSACMHCP seg106R (SEQ ID NO: 246) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44-46, Table 7, above, “Tissue samples in normal panel”), to obtain a value of relative expression for each sample relative to median of the heart samples.

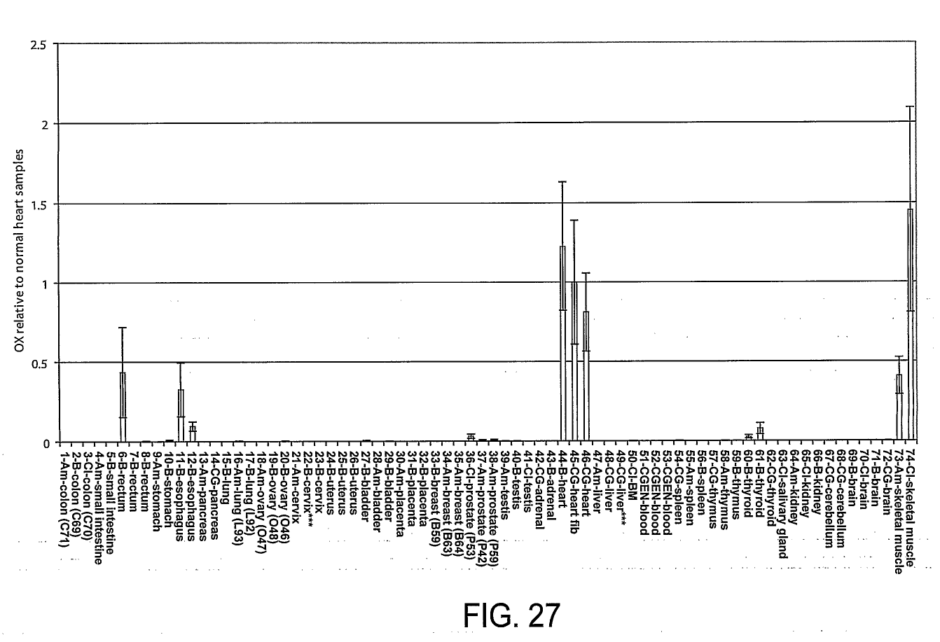

FIG. 27 is a histogram showing relative expression of the above-indicated Homo sapiens myosin, heavy polypeptide 6, cardiac muscle, alpha (cardiomyopathy, hypertrophic 1) (MYH6) transcripts in heart tissue samples as opposed to other tissues. Values represent the average of duplicate experiments. Error bars indicate the minimal and maximal values obtained

As is evident from FIG. 27, the expression of Homo sapiens myosin, heavy polypeptide 6, cardiac muscle, alpha (cardiomyopathy, hypertrophic 1) (MYH6) transcripts detectable by the above amplicon in heart tissue samples was significantly higher than in most of the other samples (Sample Nos. 1-43, 47-72 Table 7, “Tissue samples in normal panel”), except for the skeletal muscle samples.

Primer pairs are also optionally and preferably encompassed within the present invention; for example, for the above experiment, the following primer pair was used as a non-limiting illustrative example only of a suitable primer pair: HSACMHCP seg106F (SEQ ID NO: 245) forward primer; and HSACMHCP seg106R (SEQ ID NO: 246) reverse primer.

The present invention also preferably encompasses any amplicon obtained through the use of any suitable primer pair; for example, for the above experiment, the following amplicon was obtained as a non-limiting illustrative example only of a suitable amplicon: HSACMHCP seg106 (SEQ ID NO: 247).

Primers:

Forward primer HSACMHCP seg106F (SEQ ID NO:245): CCGCCATGATGGCAGAG Reverse primer HSACMHCP seg106R (SEQ ID NO:246): CCGGTGCTGCAGGTCCT Amplicon HSACMHCP seg106 (SEQ ID NO:247): CCGCCATGATGGCAGAGGAGCTGAAGAAGGAGCAGGACACCAGCGCCCACCTGGAGCGCATG AAGAAGAACATGGAGCAGACCATTAAGGACCTGCAGCACCGG

Expression of HSACMHCP HSACMHCP transcripts which are detectable by amplicon as depicted in sequence name HSACMHCP seg46 (SEQ ID NO:250) specifically in heart tissue: Expression of HSACMHCP transcripts detectable by or according to seg46-HSACMHCP seg46 (SEQ ID NO:250) amplicon and primers HSACMHCP seg46F (SEQ ID NO:248) and HSACMHCP seg46R (SEQ ID NO:249) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44-46, Table 7, above, “Tissue samples in normal panel”), to obtain a value of relative expression for each sample relative to median of the heart samples.

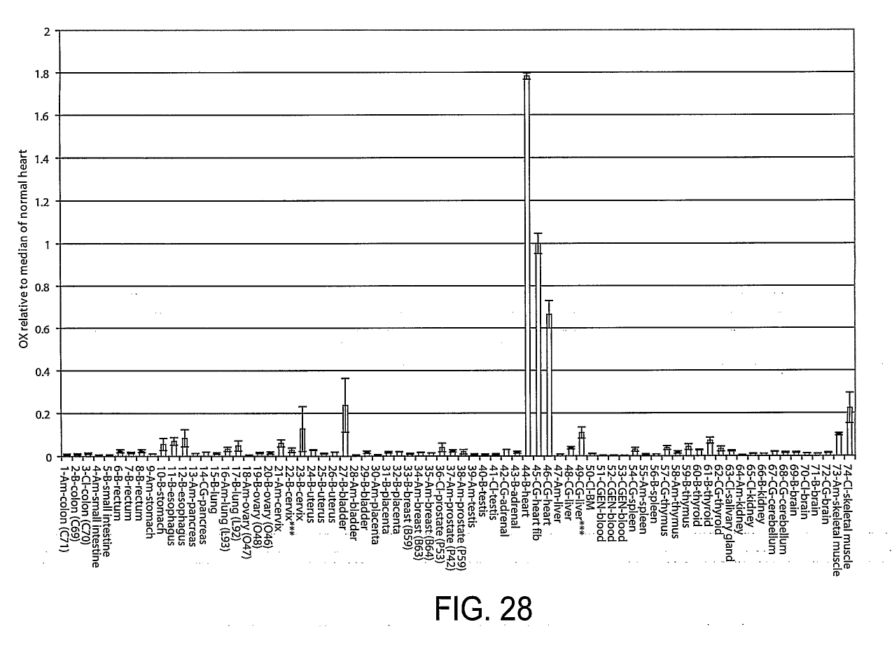

FIG. 28 is a histogram showing relative expression of the above-indicated HSACMHCP transcripts in heart tissue samples as opposed to other tissues. Values represent the average of duplicate experiments. Error bars indicate the minimal and maximal values obtained

As is evident from FIG. 28, the expression of HSACMHCP transcripts detectable by the above amplicon in heart tissue samples was significantly higher than in the other samples (Sample Nos. 1-43, 47-74 Table 7, “Tissue samples in normal panel”), including the skeletal muscle samples.

Primer pairs are also optionally and preferably encompassed within the present invention; for example, for the above experiment, the following primer pair was used as a non-limiting illustrative example only of a suitable primer pair: HSACMHCP seg46F (SEQ ID NO:248) forward primer; and HSACMHCP seg46R (SEQ ID NO:249) reverse primer.

The present invention also preferably encompasses any amplicon obtained through the use of any suitable primer pair; for example, for the above experiment, the following amplicon was obtained as a non-limiting illustrative example only of a suitable amplicon: HSACMHCP seg46 (SEQ ID NO:250).

Primers:

| | |
|---|---|
| Forward primer HSACMHCP seg46F: | (SEQ ID NO: 248) |
| CTTTCTCCAGGCCCAGCTTC | |
| Reverse primer HSACMHCP seg46R: | (SEQ ID NO: 249) |
| AAAGTGCAAGGTCCTGGGAA | |
| Amplicon HSACMHCP seg46: | (SEQ ID NO: 250) |

CTTTCTCCAGGCCCAGCTTCTCCCCACTGTGAAGTCATGGGCATGAACAGGATGATCCCCCCA
CTCTTCCTTTCCCAGGACCTTGCACTTT

### Description for Cluster HSCREACT

Cluster HSCREACT features 10 transcript(s) and 55 segment(s) of interest, the names for which are given in Tables 94 and 95 respectively. The selected protein variants are given in table 96.

TABLE 94
Transcripts of interest
Transcript Name

HSCREACT_PEA_1_T12 (SEQ ID NO: 251)
HSCREACT_PEA_1_T13 (SEQ ID NO: 252)
HSCREACT_PEA_1_T15 (SEQ ID NO: 253)
HSCREACT_PEA_1_T22 (SEQ ID NO: 254)
HSCREACT_PEA_1_T29 (SEQ ID NO: 255)
HSCREACT_PEA_1_T30 (SEQ ID NO: 256)
HSCREACT_PEA_1_T32 (SEQ ID NO: 257)
HSCREACT_PEA_1_T33 (SEQ ID NO: 258)
HSCREACT_PEA_1_T38 (SEQ ID NO: 259)
HSCREACT_PEA_1_T39 (SEQ ID NO: 260)

TABLE 95
Segments of interest

| Segment Name | Corresponding Transcript(s) |
|---|---|
| HSCREACT_PEA_1_node_63 (SEQ ID NO: 261) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_2 (SEQ ID NO: 262) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_3 (SEQ ID NO: 263) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_4 (SEQ ID NO: 264) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_5 (SEQ ID NO: 265) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_8 (SEQ ID NO: 266) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_9 (SEQ ID NO: 267) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_10 (SEQ ID NO: 268) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_11 (SEQ ID NO: 269) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_12 (SEQ ID NO: 270) | HSCREACT_PEA_1_T13 (SEQ ID |
| | NO: 252), HSCREACT_PEA_1_T15 |
| | (SEQ ID NO: 253), |
| | HSCREACT_PEA_1_T22 (SEQ ID |
| | NO: 254), HSCREACT_PEA_1_T29 |
| | (SEQ ID NO: 255), |
| | HSCREACT_PEA_1_T30 (SEQ ID |
| | NO: 256) and HSCREACT_PEA_1_T38 |
| | (SEQ ID NO: 259) |
| HSCREACT_PEA_1_node_13 (SEQ ID NO: 271) | HSCREACT_PEA_1_T22 (SEQ ID |
| | NO: 254) |
| HSCREACT_PEA_1_node_14 (SEQ ID NO: 272) | HSCREACT_PEA_1_T22 (SEQ ID |
| | NO: 254) |
| HSCREACT_PEA_1_node_15 (SEQ ID NO: 273) | HSCREACT_PEA_1_T22 (SEQ ID |
| | NO: 254) |
| HSCREACT_PEA_1_node_16 (SEQ ID NO: 274) | HSCREACT_PEA_1_T22 (SEQ ID |
| | NO: 254) |
| HSCREACT_PEA_1_node_17 (SEQ ID NO: 275) | HSCREACT_PEA_1_T22 (SEQ ID |
| | NO: 254) |
| HSCREACT_PEA_1_node_18 (SEQ ID NO: 276) | HSCREACT_PEA_1_T22 (SEQ ID |
| | NO: 254) |
| HSCREACT_PEA_1_node_19 (SEQ ID NO: 277) | HSCREACT_PEA_1_T22 (SEQ ID |
| | NO: 254) |
| HSCREACT_PEA_1_node_20 (SEQ ID NO: 278) | HSCREACT_PEA_1_T22 (SEQ ID |
| | NO: 254) |
| HSCREACT_PEA_1_node_21 (SEQ ID NO: 279) | HSCREACT_PEA_1_T22 (SEQ ID |
| | NO: 254) |
| HSCREACT_PEA_1_node_22 (SEQ ID NO: 280) | HSCREACT_PEA_1_T22 (SEQ ID |
| | NO: 254) |
| HSCREACT_PEA_1_node_23 (SEQ ID NO: 281) | HSCREACT_PEA_1_T22 (SEQ ID |
| | NO: 254) |
| HSCREACT_PEA_1_node_24 (SEQ ID NO: 282) | HSCREACT_PEA_1_T22 (SEQ ID |
| | NO: 254) |
| HSCREACT_PEA_1_node_30 (SEQ ID NO: 283) | HSCREACT_PEA_1_T12 (SEQ ID |
| | NO: 251), HSCREACT_PEA_1_T13 |
| | (SEQ ID NO: 252), |
| | HSCREACT_PEA_1_T32 (SEQ ID |
| | NO: 257) and HSCREACT_PEA_1_T39 |
| | (SEQ ID NO: 260) |
| HSCREACT_PEA_1_node_31 (SEQ ID NO: 284) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T30 |
| | (SEQ ID NO: 256), |
| | HSCREACT_PEA_1_T32 (SEQ ID |
| | NO: 257), HSCREACT_PEA_1_T38 |
| | (SEQ ID NO: 259) and |
| | HSCREACT_PEA_1_T39 (SEQ ID |
| | NO: 260) |
| HSCREACT_PEA_1_node_32 (SEQ ID NO: 285) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T30 |
| | (SEQ ID NO: 256), |
| | HSCREACT_PEA_1_T32 (SEQ ID |
| | NO: 257), HSCREACT_PEA_1_T38 |
| | (SEQ ID NO: 259) and |
| | HSCREACT_PEA_1_T39 (SEQ ID |
| | NO: 260) |
| HSCREACT_PEA_1_node_33 (SEQ ID NO: 286) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T30 |
| | (SEQ ID NO: 256), |
| | HSCREACT_PEA_1_T32 (SEQ ID |
| | NO: 257), HSCREACT_PEA_1_T38 |
| | (SEQ ID NO: 259) and |
| | HSCREACT_PEA_1_T39 (SEQ ID |
| | NO: 260) |
| HSCREACT_PEA_1_node_34 (SEQ ID NO: 287) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T30 |
| | (SEQ ID NO: 256), |
| | HSCREACT_PEA_1_T32 (SEQ ID |
| | NO: 257), HSCREACT_PEA_1_T38 |
| | (SEQ ID NO: 259) and |
| | HSCREACT_PEA_1_T39 (SEQ ID |
| | NO: 260) |
| HSCREACT_PEA_1_node_35 (SEQ ID NO: 288) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T30 |
| | (SEQ ID NO: 256), |
| | HSCREACT_PEA_1_T32 (SEQ ID |
| | NO: 257), HSCREACT_PEA_1_T38 |
| | (SEQ ID NO: 259) and |
| | HSCREACT_PEA_1_T39 (SEQ ID |
| | NO: 260) |
| HSCREACT_PEA_1_node_36 (SEQ ID NO: 289) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_37 (SEQ ID NO: 290) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_38 (SEQ ID NO: 291) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_39 (SEQ ID NO: 292) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_40 (SEQ ID NO: 293) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_41 (SEQ ID NO: 294) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253). |
| HSCREACT_PEA_1_node_42 (SEQ ID NO: 295) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_43 (SEQ ID NO: 296) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_44 (SEQ ID NO: 297) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_45 (SEQ ID NO: 298) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_46 (SEQ ID NO: 299) | HSCREACT_PEA_1_T12 (SEQ ID |
| | NO: 251), HSCREACT_PEA_1_T13 |
| | (SEQ ID NO: 252) and |
| | HSCREACT_PEA_1_T15 (SEQ ID |
| | NO: 253) |
| HSCREACT_PEA_1_node_47 (SEQ ID NO: 300) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253) and HSCREACT_PEA_1_T22 |
| | (SEQ ID NO: 254) |
| HSCREACT_PEA_1_node_48 (SEQ ID NO: 301) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253) and HSCREACT_PEA_1_T22 |
| | (SEQ ID NO: 254) |
| HSCREACT_PEA_1_node_49 (SEQ ID NO: 302) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253) and HSCREACT_PEA_1_T22 |
| | (SEQ ID NO: 254) |
| HSCREACT_PEA_1_node_50 (SEQ ID NO: 303) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253) and HSCREACT_PEA_1_T22 |
| | (SEQ ID NO: 254) |
| HSCREACT_PEA_1_node_51 (SEQ ID NO: 304) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253) and HSCREACT_PEA_1_T22 |
| | (SEQ ID NO: 254) |
| HSCREACT_PEA_1_node_52 (SEQ ID NO: 305) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253) and HSCREACT_PEA_1_T22 |
| | (SEQ ID NO: 254) |
| HSCREACT_PEA_1_node_53 (SEQ ID NO: 306) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257) and HSCREACT_PEA_1_T33 |
| | (SEQ ID NO: 258) |
| HSCREACT_PEA_1_node_54 (SEQ ID NO: 307) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257) and HSCREACT_PEA_1_T33 |
| | (SEQ ID NO: 258) |
| HSCREACT_PEA_1_node_55 (SEQ ID NO: 308) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257) and HSCREACT_PEA_1_T33 |
| | (SEQ ID NO: 258) |
| HSCREACT_PEA_1_node_56 (SEQ ID NO: 309) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257) and HSCREACT_PEA_1_T33 |
| | (SEQ ID NO: 258) |
| HSCREACT_PEA_1_node_57 (SEQ ID NO: 310) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257) and HSCREACT_PEA_1_T33 |
| | (SEQ ID NO: 258) |
| HSCREACT_PEA_1_node_58 (SEQ ID NO: 311) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258) and |
| | HSCREACT_PEA_1_T39 (SEQ ID |
| | NO: 260) |
| HSCREACT_PEA_1_node_59 (SEQ ID NO: 312) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_60 (SEQ ID NO: 313) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_61 (SEQ ID NO: 314) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |
| HSCREACT_PEA_1_node_64 (SEQ ID NO: 315) | HSCREACT_PEA_1_T12 (SEQ ID |
| | NO: 251), HSCREACT_PEA_1_T13 |
| | (SEQ ID NO: 252), |
| | HSCREACT_PEA_1_T15 (SEQ ID |
| | NO: 253), HSCREACT_PEA_1_T22 |
| | (SEQ ID NO: 254), |
| | HSCREACT_PEA_1_T29 (SEQ ID |
| | NO: 255), HSCREACT_PEA_1_T30 |
| | (SEQ ID NO: 256), |
| | HSCREACT_PEA_1_T32 (SEQ ID |
| | NO: 257), HSCREACT_PEA_1_T33 |
| | (SEQ ID NO: 258), |
| | HSCREACT_PEA_1_T38 (SEQ ID |
| | NO: 259) and HSCREACT_PEA_1_T39 |
| | (SEQ ID NO: 260) |

TABLE 96
Proteins of interest

| Protein Name | Corresponding Transcript(s) |
|---|---|
| HSCREACT_PEA_1_P9 (SEQ ID NO: 317) | HSCREACT_PEA_1_T12 (SEQ ID NO: 251); |
| | HSCREACT_PEA_1_T32 (SEQ ID NO: 257); |
| | HSCREACT_PEA_1_T39 (SEQ ID NO: 260) |
| HSCREACT_PEA_1_P10 (SEQ ID NO: 318) | HSCREACT_PEA_1_T13 (SEQ ID NO: 252) |
| HSCREACT_PEA_1_P12 (SEQ ID NO: 319) | HSCREACT_PEA_1_T15 (SEQ ID NO: 253); |
| | HSCREACT_PEA_1_T30 (SEQ ID NO: 256); |
| | HSCREACT_PEA_1_T38 (SEQ ID NO: 259) |
| HSCREACT_PEA_1_P16 (SEQ ID NO: 320) | HSCREACT_PEA_1_T22 (SEQ ID NO: 254) |
| HSCREACT_PEA_1_P22 (SEQ ID NO: 321) | HSCREACT_PEA_1_T29 (SEQ ID NO: 255) |
| HSCREACT_PEA_1_P28 (SEQ ID NO: 322) | HSCREACT_PEA_1_T33 (SEQ ID NO: 258) |

These sequences are variants of the known protein C-reactive protein precursor (SEQ ID NO:316) (SwissProt accession identifier CRP_HUMAN (SEQ ID NO:316)), referred to herein as the previously known protein.

Protein C-reactive protein precursor (SEQ ID NO:316) is known or believed to have the following function(s): Displays several functions associated with host defense: it promotes agglutination, bacterial capsular swelling, phagocytosi and complement fixation through its calcium-dependent binding to phosphorylcholine. Can interact with DNA and histones and may scavenge nuclear material released from damaged circulating cells. Known polymorphisms for this sequence are as shown in Table 97.

TABLE 97
Amino acid mutations for Known Protein

| SNP position(s) on amino | |
|---|---|
| acid sequence | Comment |
| 49 | K -> G |
| 52 | T -> G |
| 67-82 | YSIFSYATKRQDNEIL -> |
| | TVFSRMPPRDKTMRFF |
| 80-98 | Missing |
| 170 | L -> V |

Protein C-reactive protein precursor (SEQ ID NO:316) localization is believed to be Secreted.

The previously known protein also has the following indication(s) and/or potential therapeutic use(s): Infarction, myocardial; Surgery adjunct; Coronary artery bypass grafting; Systemic inflammatory response syndrome. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Polymorphonuclear neutrophil inhibitor. A therapeutic role for a protein represented by the cluster has been predicted. The cluster was assigned this field because there was information in the drug database or the public databases (e.g., described herein above) that this protein, or part thereof, is used or can be used for a potential therapeutic indication: Septic shock treatment; Cardiovascular.

The following GO Annotation(s) apply to the previously known protein. The following annotation(s) were found: acute-phase response; inflammatory response, which are annotation(s) related to Biological Process; ligand binding or carrier, which are annotation(s) related to Molecular Function; and extracellular space, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of HSCREACT may optionally have one or more of the following utilities, as described with regard to Table 98 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 98
Utilities for Variants of HSCREACT, related to C-reactive protein precursor (SEQ ID
NO: 316) isoform: CRP_HUMAN (SEQ ID NO: 316)

| Utility | Reason | Reference |
|---|---|---|
| A risk factor for atherosclerosis | elevated CRP concentration | Vnitr Lek. 2004 |
| progression and its complications | | Nov; 50(11): 852-7. |
| (myocardial infarction and stroke) | | |
| prognostic marker in acute coronary | elevated CRP concentration | Am Heart Hosp J. 2004 |
| syndromes, following angioplasty, and | | Fall; 2(4 Suppl 1): 4-9. |
| in the long-term management of post- | | |
| infarction patients. | | |
| myocardial infarction risk factor | elevated CRP concentration | J Cardiovasc Nurs. 2004 Nov-Dec; |
| | | 19(6): 425-9. |
| nonlipid cardiovascular event risk | elevated CRP concentration | J Cardiovasc Nurs. 2004 Nov-Dec; |
| marker | | 19(6): 425-9. |
| plasma level of CRP is considered to | elevated plasma level of CRP | Curr Opin Lipidol. 2004 |
| reflect the inflammatory condition of | | Dec; 15(6): 651-7. |
| the patient and/or the vessel wall | | |
| a marker for inflammatory bowel | serum levels of CRP correlate | Inflamm Bowel Dis. 2004 |
| disease. | well with disease activity and | Sep; 10(5): 661-5. |
| | with other markers of | |
| | inflammation as the CDAI, | |
| | serum amyloid, IL-6 and faecal | |
| | calprotectin. | |
| a valuable marker to detect and follow | serum levels of CRP correlate | Inflamm Bowel Dis. 2004 |
| up disease activity in Crohn's disease | well with disease activity and | Sep; 10(5): 661-5. |
| (CD). | with other markers of | |
| | inflammation as the CDAI, | |
| | serum amyloid, IL-6 and faecal | |
| | calprotectin. | |
| active mediator in the pathogenesis of | macrophage recruitment, and | Am J Med. 2004 Oct |
| atherosclerosis. | foam cell generation | 1; 117(7): 499-507. |
| Serum procalcitonin and C-reactive | elevated plasma level of CRP | Clin Infect Dis. 2004 Jul |
| protein levels as markers of bacterial | | 15; 39(2): 206-17. Epub 2004 |
| infection | | Jul 02. |
| a marker to identify women at risk for | elevated CRP concentration | J Fam Pract. 2004 |
| heart disease After menopause. | | Jul; Suppl: S18-24. |
| prediction of type 2 diabetes. | elevated CRP concentration | Circulation. 2004 Jun |
| | | 15; 109(23): 2818-25. |
| ischemic heart disease in peritoneal | elevated CRP concentration | Nephrology (Carlton). 2003 |
| dialysis (PD) patients. | | Oct; 8 Suppl: S40-4. |
| Anti-myeloperoxidase (anti-MPO) | C-reactive protein and anti- | Ren Fail. 2003 |
| associated vasculitis | MPO titer, have substantial | Nov; 25(6): 1057-66. |
| | values in monitoring disease | |
| | activity | |
| diagnosis of sepsis in full-term or near- | elevated hsCRP concentration | Adv Neonatal Care. 2003 |
| term infants. | | Feb; 3(1): 3-13. |
| surrogate endpoint in cardiovascular | Various cardiovascular drugs, | Vascul Pharmacol. 2002 |
| disease treatments. | notably statins, can induce a | Aug; 39(3): 99-104. |
| | marked decrease in low levels | |
| | of CRP | |
| cardiac hypertrophy | elevated CRP concentration | Am J Kidney Dis. 2002 |
| | | Dec; 40(6): 1340-1. |
| a marker for all-cause and | elevated CRP concentration | Nephrol Dial Transplant. |
| cardiovascular mortality in | | 2002; 17 Suppl 8: 29-32; |
| haemodialysis patients. | | discussion 39-40. |
| a prognostic indicator in advanced | a predictor of survival has been | Curr Oncol Rep. 2002 |
| cancer. | shown in multiple myeloma, | May; 4(3): 250-5. |
| | melanoma, lymphoma, ovarian, | |
| | renal, pancreatic, and | |
| | gastrointestinal tumors. | |
| secondary prevention of coronary | elevated CRP concentration | Clin Chim Acta. 2001 Sep |
| events. | | 15; 311(1): 49-52. |
| primary prevention of ischemic heart | elevated CRP concentration | Clin Chim Acta. 2001 Sep |
| disease. | | 15; 311(1): 45-8. |
| a marker of chronic inflammation in | elevated CRP concentration | Blood Purif. 2000; 18(3): 183-90. |
| uremic patients. | | |
| marker of risk of restenosis | elevated CRP concentration | Semin Interv Cardiol. 1999 |
| | | Sep; 4(3): 105-10. |
| monitor IL-2 cancer immunotherapy | abnormally high pretreatment | J Biol Regul Homeost Agents. |
| | values of inflammatory | 1999 Apr-Jun; 13(2): 110-4. |
| | response markers, including | |
| | ESR, CRP, neopterin and SIL- | |
| | 2R, may predict a lack of IL-2 | |
| | efficacy | |
| cardiovascular disease risk. | elevated CRP levels (as | Cleve Clin J Med. 2003 |
| | measured by highly sensitive | Jul; 70(7): 634-40. |
| | assays) are associated with | |
| | increased cardiovascular risk. | |
| | Of patients at intermediate risk | |
| | and at high risk | |
| human breast cancer | Secondary marker combined | In Vivo. 1993 Nov-Dec; |
| | with CEA | 7(6B): 607-13. |
| predict the prognosis, to monitor the | non-specific tumor markers | Nippon Rinsho. 1996 |
| effects of the treatment, to detect | such as LDH, ESR, CRP and | Jun; 54(6): 1621-5. |
| minimum residual disease and to | beta 2-MG are generally used | |
| diagnose early relapse in patients' with | | |
| peripheral blood with | | |
| lymphoproliferative disorders, | | |
| especially malignant lymphomas. | | |
| Predictors of joint damage in | Unspecific markers of | APMIS. 1996 Feb; 104(2): 81-93. |
| rheumatoid arthritis. | inflammation, notably ESR or | |
| | CRP when persistently | |
| | elevated, are useful to monitor | |
| | disease course | |
| rheumatoid arthritis. | C-reactive protein (CRP) is a | Cell Mol Biol (Noisy-le- |
| | sensitive marker, and is used to | grand). 1995 Dec; 41(8): 993-1006. |
| | monitor inflammatory and | |
| | infectious diseases. In | |
| | rheumatoid arthritis (RA), CRP | |
| | correlates with disease activity | |
| | and response to therapy, and | |
| | CRP levels are influenced by | |
| | disease-modifying drugs and | |
| | corticosteroids. | |
| C-reactive protein: a biomarker of | CRP (p = 0.002) and DFI (p = | J Urol. 2005 Jan; 173(1): 52-5. |
| survival in patients with metastatic | 0.0497) were found to have an | |
| renal cell carcinoma treated with | independent role in survival. | |
| subcutaneous interleukin-2 based | When we correlated clinical | |
| immunotherapy. | and biohumoral factors, only | |
| | CRP correlated with DFI (p = | |
| | 0.021) and prior nephrectomy | |
| | (p = 0.041). | |
| Diagnosing melanoma patients entering | CRP yielded a sensitivity of | Br J Cancer. 2004 Aug |
| American Joint Committee on Cancer | 0.769 together with a | 16; 91(4): 699-702. |
| stage IV, C-reactive protein in serum is | specificity of 0.904 in | |
| superior to lactate dehydrogenase. | diagnosing AJCC stage IV | |
| | entry. | |
| Increased C-reactive protein levels in | elevated CRP concentration | J Clin Endocrinol Metab. 2004 |
| the polycystic ovary syndrome: a | | May; 89(5): 2160-5. |
| marker of cardiovascular disease. | | |
| type 2 diabetes | Elevated CRP levels are a | Diabetes. 2004 |
| | strong independent predictor of | Mar; 53(3): 693-700. |
| | type 2 diabetes and may | |
| | mediate associations of TNF- | |
| | alphaR2 and IL-6 with type 2 | |
| | diabetes. | |
| esophageal squamous cell carcinoma. | A high CRP level is associated | J Surg Oncol. 2003 |
| | with tumor progression and | Aug; 83(4): 248-52. |
| | poor survival in patients with | |
| | esophageal squamous cell | |
| | carcinoma. | |
| CRP holds promise as a diagnostic and | CRP levels were correlated | Med Pediatr Oncol. 2003 |
| prognostic index and follow-up monitor | with stage, absence or presence | Jul; 41(1): 21-5. |
| in pediatric and adolescent patients with | of B symptoms, and prognosis | |
| Hodgkin disease | | |
| microalbuminuria in patients with non- | a high frequency of | Br J Haematol. 2003 |
| Hodgkin's lymphoma | microalbuminuria (>or=20 microg/min) | Apr; 121 (2): 275-9. |
| | and UAE | |
| | correlated strongly with serum | |
| | levels of CRP, IL-6 and TNF- | |
| | alpha. UAE, CRP, IL-6 and | |
| | TNF-alpha were significantly | |
| | higher in patients with | |
| | advanced disease stage, B | |
| | symptoms and in high-risk | |
| | patients according to the | |
| | International Prognostic Index. | |
| lung cancer | CYFRA 21-1, NSE, and CRP | Int J Clin Oncol. 2002 |
| | yielded an increase in | Jun; 7(3): 145-51. |
| | sensitivity of approximately | |
| | 20%, i.e., 92%, compared with | |
| | that of the best single marker | |
| Correlation between the uptake of Tc- | a positive correlation between | Clin Lab Haematol. 2002 |
| 99m-sestaMIBI and prognostic factors | Tc-99m-MIBI intensity and C- | Jun; 24(3): 155-9. |
| in patients with multiple myeloma. | reactive protein (CRP; r = 0.506, | |
| | P < 0.01), erythrocyte | |
| | sedimentation rate (ESR; | |
| | r = 0.368, P < 0.05), beta2- | |
| | microglobulin (beta2M; | |
| | r = 0.749, P < 0.001), | |
| | interleukin-6 (IL-6; r = 0.823, P < 0.001), | |
| | soluble Interleukin-6 | |
| | receptor (sIL-6r; r = 0.806, P < | |
| | 0.001), serum calcium | |
| | (r = 0.578, P < 0.001) and bone | |
| | alkaline phosphatase (BAP; | |
| | r = 0.472, P < 0.01). | |
| malignant fibrous histiocytoma | Patients with MFH frequently | Int J Cancer. 2002 May |
| | present with an inflammatory | 10; 99(2): 167-70. |
| | PNS, such as elevated serum | |
| | CRP level, which can be a | |
| | useful marker of disease | |
| | activity and a valuable | |
| | prognostic indicator. | |
| IL-6 especially in the combination with | The levels of IL-6, CRP and | Pol Merkuriusz Lek. 2001 |
| CRP and TPS may be useful in the | TPS were decreased above cut | Sep; 11(63): 210-3. |
| diagnosis and the evaluation of therapy | off values in patients with | |
| of patients with ovarian carcinoma | remission, and did not changed | |
| | in patients with progression and | |
| | stabilization disease | |
| Not traditional prognostic factors in | C-reactive protein, ferritin and | Minerva Urol Nefrol. 2001 |
| human conventional renal carcinoma | the proliferative activity | Dec; 53(4): 211-9. |
| | indexes (Ki67 and AgNOR) | |
| | appear to be, at present, the best | |
| | prognostic tools. | |
| type 2 diabetes mellitus | Elevated levels of CRP and IL- | JAMA. 2001 Nov |
| | 6 predict the development of | 14; 286(18): 2233. |
| | type 2 DM | |
| hepatocellular carcinoma in patients | very high values of CRP in | J Gastroenterol Hepatol. 2000 |
| with cirrhosis | patients with cirrhosis may | Apr; 15(4): 417-21. |
| | suggest the presence of a | |
| | diffuse-type HCC. | |
| tubo-ovarian complex (TOC). | Determination of CRP, and | Int J Gynaecol Obstet. 1999 |
| | somewhat less ESR, is more | Mar; 64(3): 273-9. |
| | suitable for following the | |
| | treatment of TOC in the acute | |
| | phase while CA-125 and | |
| | measuring of TOC size is more | |
| | suitable in the subacute phase. | |
| C-reactive protein serum level is a | there is a significant | Leuk Lymphoma. 1998 |
| valuable and simple prognostic marker | relationship between CRP and | Oct; 31(3-4): 351-7 |
| in non Hodgkin's lymphoma | IL-6 levels (p < 0.00017), and | |
| | CRP levels and B symptoms | |
| | (p < 0.001). | |
| indicator for prognosis in colorectal | A preoperative serum elevation | Am J Surg. 1998 |
| cancer. | of CRP was thus found to be an | Oct; 176(4): 335-8. |
| | indicator of the malignant | |
| | potential of the tumor as well as | |
| | a predictor of the prognosis of | |
| | patients with colorectal cancer. | |
| advanced epithelial ovarian cancer. | high IL-6 and/or CRP serum | Gynecol Oncol. 1998 |
| | levels may represent an | Jun; 69(3): 248-52. |
| | important and independent | |
| | prognostic factor of the likely | |
| | outcome in cancer patients. | |
| heterotopic ossification after spinal cord | erum CRP is a useful and more | J Spinal Cord Med. |
| injury. | specific test than is ESR for | 2004; 27(5): 434-7. |
| | monitoring the inflammatory | |
| | activity of HO after SCI. The | |
| | normalization of CRP was seen | |
| | during the first 3 to 4 weeks of | |
| | etidronate therapy, indicating a | |
| | resolution of acute-phase | |
| | inflammatory reaction. | |
| Autoimmune pancreatitis with | The serum amylase, C-reactive | J Gastroenterol. 2004 |
| pseudocysts | protein (CRP), and IgG | Oct; 39(10): 1005-10. |
| | (especially IgG4) were | |
| | elevated, and abdominal | |
| | computed tomography (CT) | |
| | revealed diffuse enlargement of | |
| | the pancreas and pseudocysts. | |
| a prognostic role for C-reactive protein | The response of SAA, CRP and | Scand J Gastroenterol. 2004 |
| and hepatocyte growth factor after | IGF-I was lower in PH patients | Nov; 39(11): 1141-8. |
| laparotomy or partial hepatectomy in | than in laparotomy patients, A | |
| patients with colorectal liver metastases | higher HGF and CRP response | |
| | was associated with a poorer | |
| | prognosis. | |
| coronary artery ectasia (CAE) | Elevated CRP levels | Am J Cardiol. 2004 Nov |
| | | 15; 94(10): 1303-6. |
| diagnostic value of C-reactive protein, | Evaluation of the clinical | Saudi Med J. 2004 |
| white blood cell count and neutrophil | symptoms and signs combined | Sep; 25(9): 1212-5. |
| percentage in childhood appendicitis. | with the triple tests can improve | J Med Assoc Thai. 2004 |
| | the diagnostic accuracy and | Mar; 87(3): 296-303. |
| | significantly reduce the | |
| | incidence of perforation and the | |
| | rate of negative laparotomy. | |
| C-reactive protein as a predictor of | | Neuro Endocrinol Lett. 2004 |
| threatening preterm delivery. | | Aug; 25(4): 302-6. |
| C-reactive protein as a prognostic | Increased serum CRP was | Dig Dis Sci. 2004 |
| variable that reflects uncontrolled up- | associated with recurrent | Jun; 49(6): 970-6. |
| regulation of the IL-1-IL-6 network | disease and shorter survival | |
| system in colorectal carcinoma. | time | |
| -reactive protein levels and the | CRP levels are elevated in | Circulation. 2004 Aug |
| expansion of screen-detected abdominal | larger aneurysms but do not | 17; 110(7): 862-6. Epub 2004 |
| aortic aneurysms in men. | appear to be associated with | Aug 09. |
| | rapid expansion. The most | |
| | useful predictor of aneurysmal | |
| | expansion in men is aortic | |
| | diameter. | |
| The predictive and diagnostic values of | PCT and CRP are comparable | J Chin Med Assoc. 2004 |
| procalcitonin and C-reactive protein for | with each other in prediction of | May; 67(5): 217-21. |
| clinical outcome in febrile neutropenic | the clinical severity of febrile | |
| patients. | neutropenic attacks. | |
| | Furthermore, serum CRP levels | |
| | correlate with the duration of | |
| | fever. | |
| C-reactive protein as an indicator of | Daily measurement of CRP is | Intensive Care Med. 1998 |
| sepsis. | useful in the detection of sepsis | Oct; 24(10): 1052-6. |
| | and it is more sensitive than the | |
| | currently used markers, such as | |
| | BT and WBC. | |
| CRP as en early and preclinical marker | difference of CRP | Ginekol Pol. 1997 |
| of postsurgery inflammation process. | concentration was detected in | Jan; 68(1): 36-40. |
| | 24th hour after operation | |
| prediction of severity of acute | The serum concentration of IL- | J Chin Med Assoc. 2004 |
| pancreatitis | 6 on the first day and/or the | Sep; 67(9): 442-6. |
| | serum concentration of CRP on | Gastroenterol Clin North Am. |
| | the 2nd day of admission are | 2004 Dec; 33(4): 871-90. |
| | useful for early prediction of | |
| | the severity of acute | |
| | pancreatitis. | |
| Severe acute pancreatitis (SAP) and | CRP levels differed | Clin Chem Lab Med. 2004 |
| mild acute pancreatitis (MAP) | significantly on days 2-5 | May; 42(5): 549-55. |
| Behcet's disease and risk of venous | protein S, AT, alpha(1)- | Br J Haematol. 2004 |
| thrombosis | antitrypsin, fibrinogen, factor | Aug; 126(4): 550-6. |
| | VIII, VWF and CRP levels | J Eur Acad Dermatol |
| | were significantly higher in | Venereol. 2004 |
| | patients than in controls | May; 18(3): 318-20. |
| | serum CRP and IL-6 levels are | |
| | elevated in patients with active | |
| | BD | |
| ankylosing spondylitis | data suggest that CRP is a | South Med J. 2004 |
| | better marker of disease activity | Apr; 97(4): 350-3. |
| | than ESR, Hp, and beta2MG. | |
| C-reactive protein as a predictor of | | Arthritis Rheum. 2005 |
| infliximab treatment outcome in | | Jan; 52(1): 42-8. |
| patients with rheumatoid arthritis: | | |
| defining subtypes of nonresponse and | | |
| subsequent response to etanercept. | | |
| Does the type of hormone replacement | The 2 forms of HRT therapy | |
| therapy affect C-reactive protein levels | differ in effect, which is | |
| in postmenopausal women | expressed as a change in CRP | |
| | concentration. A tendency to | |
| | increase CRP values when | |
| | using oral HRT is observed, | |
| | while such an effect is not | |
| | observed in case of transdermal | |
| | therapy after 3 months. | |
| joint inflammatory activity in RA | ultrasonography findings | Ann Rheum Dis. 2005 |
| | correlated better with CRP and | Mar; 64(3): 375-81. |
| | ESR to compare the clinical | |
| | assessment of overall | |
| | inflammatory activity in | |
| | patients with rheumatoid | |
| | arthritis (RA) | |
| diabetes in infants and young adults | Levels of hs CRP were 3-fold | Presse Med. 2005 Jan 29; 34(2 |
| | greater in diabetic patients | Pt 1): 89-93. |
| | without complications than in | |
| | controls and 5-fold greater in | |
| | diabetic patients with | |
| | subclinical complications. High | |
| | sensitive CRP therefore appears | |
| | to be an interesting indicator of | |
| | the risk for developing | |
| | complications. | |
| Cardiovascular risk factors in women | levels of several inflammatory | Arthritis Rheum. 2004 |
| with and without rheumatoid arthritis. | biomarkers linked to CVD were | Nov; 50(11): 3444-9. |
| | significantly elevated in women | |
| | with RA, including CRP | |
| mumps orchitis | CRP titers in mumps patients | Pediatr Infect Dis J. 2004 |
| | with orchitis and meningitis. | Oct; 23(10): 971. |
| | The serum CRP titers were | |
| | significantly higher in the | |
| | patients with orchitis than in | |
| | those with meningitis. | |
| severe closed trauma | The course of serum CRP | Enferm Infecc Microbiol Clin. |
| | levels is different in the group | 2001 Feb; 19(2): 61-5. |
| | of patients with severe blunt | |
| | trauma and infection, compared | |
| | with the non-infected group | |
| | during the first week after | |
| | injury and it could be an useful | |
| | supplementary marker for | |
| | infection after postinjury day 4. | |
| | A value of 110 mg/l or higher | |
| | for CRP should suggest an | |
| | underlying infectious | |
| | complication. | |
| Pneumonia | Serum CRP level is a useful | Chest. 2004 Apr; 125(4): 1335-42. |
| | marker for establishing the | |
| | diagnosis of CAP in adult | |
| | patients with lower respiratory | |
| | tract infections. High CRP | |
| | values are especially high in | |
| | patients with pneumonias | |
| | caused by S pneumoniae or L | |
| | pneumophila. Moreover, high | |
| | CRP values are suggestive of | |
| | severity, which may be of value | |
| | in deciding about the | |
| | appropriateness of inpatient | |
| | care. | |
| outcomes after statin therapy | Patients who have low CRP | N Engl J Med. 2005 Jan |
| | levels after statin therapy have | 6; 352(1): 20-8. |
| | better clinical outcomes than | |
| | those with higher CRP levels, | |
| | regardless of the resultant level | |
| | of LDL cholesterol. Strategies | |
| | to lower cardiovascular risk | |
| | with statins should include | |
| | monitoring CRP as well as | |
| | cholesterol. | |
| Sinusitis | The CRP rapid test has a | Br J Gen Pract. 2004 |
| | substantial influence on the | Sep; 54(506): 659-62. |
| | treatment of sinusitis, and | |
| | implementing the test in | |
| | general practice may lead to a | |
| | reduction in antibiotic | |
| | prescribing to patients with | |
| | sinusitis | |
| Post transplant diabetes mellitus and | high serum levels of CRP and | Transplantation. 2005 Feb |
| atherosclerotic events in renal | hyperhomocystinemia were | 27; 79(4): 438-43. |
| transplant recipients | found to be among the | |
| | nontraditional factors | |
| | contributing to AE | |
| Factors predisposing to pre-eclampsia | GDM-PE had elevated body | J Hypertens. 2004 |
| in women with gestational diabetes | mass index, blood pressure, | Dec; 22(12): 2371-8. |
| | fasting glucose, insulin, uric | |
| | acid, and G-reactive protein | |
| | (CRP), which have all been | |
| | linked with the ‘metabolic | |
| | syndrome’. They had a greater | |
| | degree of microalbuminuria and | |
| | more frequently reported a | |
| | family history of hypertension | |
| | and maternal gestational | |
| | diabetes. | |
| Baseline predictors of vascular events | Antiphospholipid antibodies | |
| in Systemic lupus erythematosus | and CRP support the role of | |
| | inflammation and | |
| | autoimmunity in the | |
| | development of accelerated | |
| | atherosclerosis in SLE. | |
| Atherosclerosis | A high plasma level of high- | J Atheroscler Thromb. |
| | sensitivity C-reactive protein | 2004; 11(5): 265-70. |
| | (hs-CRP) is an important | |
| | predictor of atherosclerotic | |
| | diseases. | |
| hospital-acquired severe acute | elevated levels of LDH and | Mayo Clin Proc. 2004 |
| respiratory syndrome. | CRP correlated with severe | Nov; 79(11): 1372-9. |
| | illness requiring mechanical | |
| | ventilatory support. | |
| atrial fibrillation | high levels of CRP | Am J Cardiol. 2004 Nov |
| | independently predicted an | 15; 94(10): 1255-9. |
| | increased risk of AF among a | |
| | large, prospectively studied | |
| | patient cohort that was assessed | |
| | angiographically. Increased | |
| | CRP is a new risk marker for | |
| | AF propensity, | |
| acute pancreatitis with a significant | A favourable turn in the course | Rozhl Chir. 2004 |
| multiorgan dysfunction | of the disease was signalized by | Sep; 83(9): 443-50. |
| | a drop in the serum CRP and by | |
| | a decreasing need for | |
| | vasopressors medication. | |
| acute pancreatitis | Elevated CRP levels above 130 mg/L | Gastroenterol Clin North Am. |
| | can also predict a severe | 2004 Dec; 33(4): 855-69, viii. |
| | course of acute pancreatitis | |
| bacterial peritonitis, and malignant and | The sensitivity and specificity | J Natl Med Assoc. 2005 |
| cirrhotic ascites. | of ascitic fluid CRP, TNF-alpha | Feb; 97(2): 276-80. |
| | and TNF-r values were found to | |
| | be similar. Ascitic fluid | |
| | Creactive protein to | |
| | differentiate SBP and malignant | |
| | ascitic from cirrhotic ascites are | |
| | cheap, practical and safe tests | |
| | used in the differential | |
| | diagnosis of ascites. | |
| Procalcitonin, C-reactive protein, and | serum CRP and endotoxin, | Bone Marrow Transplant. |
| endotoxin after bone marrow | correlated with the severity of | 2003 Jun; 31(12): 1137-42. |
| transplantation: identification of | sepsis (8.2 ng/ml in ‘sepsis’ and | |
| children at high risk of morbidity and | 22.3 ng/ml in ‘severe sepsis’, | |
| mortality from sepsis. | P = 0.028) and provided useful | |
| | prognostic information during | |
| | septic episodes. | |
| Pharyngitis | The CRP test seems to be a | Scand J Clin Lab Invest. |
| | valid test for monitoring the | 2002; 62(7): 521-5. |
| | clinical course in GAS | |
| | pharyngitis. | |
| paroxysmal atrial fibrillation | longer AF duration is | Heart Vessels. 2005 |
| | associated with CRP elevation | Mar; 20(2): 45-9. |
| | and atrial structural remodeling, | |
| | as approximated by larger LA | |
| | diameter. | |
| Progression of native coronary plaques | increased pre-procedural C | Heart. 2005 Apr; 91(4): 535-6. |
| and in-stent restenosis | reactive protein. | |
| predicts outcome in patients undergoing | in patients who have undergone | Br J Cancer. 2005 Jan |
| resection for ductal adenocarcinoma | potentially curative resection | 17; 92(1): 21-3. |
| head of pancreas. | for ductal adenocarcinoma of | |
| | the head of pancreas, the | |
| | presence of a systemic | |
| | inflammatory response (CRP) | |
| | predicts poor outcome. | |
| risk of colorectal cancer | C-reactive protein elevation | Gastroenterol Nurs. 2004 Sep-Oct; |
| | | 27(5): 246-7. |
| non-small-cell lung cancer | C-reactive protein, interleukin- | Br J Cancer. 2004 Dec |
| | 6, soluble gp130, soluble TNF | 13; 91(12): 1993-5. |
| | receptor, interleukin-1 receptor | |
| | antagonist and IL-8 | |
| | concentrations were | |
| | significantly elevated in cancer | |
| | patients (P < or = 0.001). Cancer | |
| | patients with elevated C- | |
| | reactive protein concentrations | |
| | had greater concentrations of | |
| | interleukin-6 (P < 0.01) and | |
| | interleukin-1 receptor | |
| | antagonist (P < 0.05). | |
| colon cancer | Plasma CRP concentrations are | JAMA. 2004 Feb |
| | elevated among persons who | 4; 291(5): 585-90. |
| | subsequently develop colon | |
| | cancer. These data support the | |
| | hypothesis that inflammation is | |
| | a risk factor for the | |
| | development of colon cancer in | |
| | average-risk individuals. | |
| breast tumors | The concentrations of IL-6 and | Pol Merkuriusz Lek. 2003 |
| | CRP were significantly | Aug; 15(86): 115-7. |
| | increased in carcinoma patients | |
| | relative to control group and to | |
| | benign breast tumor group. The | |
| | frequency of higher levels and | |
| | absolute value of IL-6 and CRP | |
| | showed tendency to significant | |
| | increase with the stage of | |
| | disease. A positive correlation | |
| | was observed between the | |
| | concentrations of IL-6 and | |
| | CRP. | |
| type 1 diabetes | elevated CRP levels in infants | Diabetes. 2004 |
| | and young children before the | Oct; 53(10): 2569-73. |
| | onset of type 1 diabetes adds to | |
| | the evidence that the disease is | |
| | an immunoinflammatory | |
| | disorder. The elevated CRP | |
| | levels may provide an | |
| | additional marker for risk of | |
| | progression to type 1 diabetes. | |
| increased risk of incident | High plasma levels of CRP | Diabetes Care. 2004 |
| cardiovascular events | were associated with an | Apr; 27(4): 889-94. |
| | increased risk of incident | |
| | cardiovascular events among | |
| | diabetic men, independent of | |
| | currently established lifestyle | |
| | risk factors, blood lipids, and | |
| | glycemic control. | |
| hypo/hyperalphalipoproteinemias | Plasma CRP levels showed a | Metabolism. 2003 |
| | particularly strong correlation | Apr; 52(4): 432-6. |
| | with plasma HDL-C | |
| | concentrations | |
| penile vascular disease | CRP levels correlate | Int J Impot Res. 2003 |
| | significantly with increasing | Aug; 15(4): 231-6. |
| | severity of penile vascular | |
| | disease as measured by penile | |
| | Doppler. | |
| ischemic events | C-reactive protein predicts | Stroke. 2003 Oct; 34(10): 2463-8. |
| | further ischemic events in first- | Epub 2003 Sep 18. |
| | ever transient ischemic attack | |
| | or stroke patients with | |
| | intracranial large-artery | |
| | occlusive disease. | |
| Peripheral vascular disease | CRP levels where found | Vasc Med. 2004 |
| | correlated with ankle-brachial | May; 9(2): 107-15 |
| | index | |

According to other optional embodiments of the present invention, variants of this cluster according to the present invention (amino acid and/or nucleic acid sequences of HSCREACT may optionally have one or more of the following utilities, some of which are related to utilities described above. It should be noted that these utilities are optionally and preferably suitable for human and non-human animals as subjects, except where otherwise noted.

A non-limiting example of such a utility is the detection, diagnosis and/or determination of clinical events such as acute coronary syndrome, in patients with signs and symptoms of suspected cardiac origin. The method comprises detecting a HSCREACT variant, for example a variant protein, protein fragment, peptide, polynucleotide, polynucleotide fragment and/or oligonucleotide as described herein, optionally and preferably in a serum sample. Use of known C reactive protein for diagnosis and risk stratification of clinical events such as acute coronary syndrome, in patients with signs and symptoms of suspected cardiac origin is described with regard to PCT Application No. WO04103150, hereby incorporated by reference as if fully set forth herein. The PCT application describes a method for diagnosis of a clinical condition in a patient or for risk stratification of a patient possibly with the condition, comprising the steps of: a) obtaining from the patient at least one sample of a substance stream which has been in contact with a tissue suspected of undergoing the clinical condition; b) conducting at least a first in vitro diagnostic assay on the sample and optionally additional in vitro diagnostic assays; c) measuring and analyzing the patient's electrocardiogram (ECG); and d) applying an algorithm to combine the results of the assay (s) of step (b) and the electrocardiogram in step (c) using an algorithm to provide a positive or negative diagnosis or risk stratification of the clinical condition. Another example of use of known C reactive protein for diagnosis and evaluation of acute coronary syndromes origin is described with regard to PCT Application No. WO02089657, hereby incorporated by reference as if fully set forth herein. The PCT application describes a method for diagnosis and evaluation of acute coronary syndromes using a panel of markers comprising one or more specific markers for myocardial injury and one or more non-specific markers for myocardial injury. A variety of markers are disclosed for assembling a panel of markers for such diagnosis and evaluation. In various aspects, the invention provides methods for the early detection and differentiation of stable angina, unstable angina, and myocardial infarction. CRP is a nonspecific marker for ACS, and elevations of the CRP concentration in plasma may occur from unrelated conditions involving activation of the immune system. Despite its high degree of non-specificity for ACS, CRP may be useful in the identification of unstable angina and AMI when used with another marker that is specific for cardiac tissue injury. Another example of use of known C reactive protein for diagnosis and distinguishing amongst a plurality of cardiovascular disorders is described with regard to US Application No. US20040253637, hereby incorporated by reference as if fully set forth herein. The US application describes a method for analyzing a subject sample for a plurality of subject-derived markers selected to distinguish amongst a plurality of cardiovascular disorders, comprising: assaying said sample for the presence or amount of one or more subject-derived markers related to blood pressure regulation, and for the presence or amount of one or more subject-derived markers related to myocardial injury, and characterizing said subject's risk of having developed or of developing each of said plurality of cardiovascular disorders based upon the presence or amount of said markers, wherein the amount of at least one of said one or more subject-derived markers is not compared to a predetermined threshold amount.

Another non-limiting example of a possible utility is the prediction, detection, diagnosis and/or determination of increased risk of system failure or mortality. The method comprises detecting a HSCREACT variant, for example a variant protein, protein fragment, peptide, polynucleotide, polynucleotide fragment and/or oligonucleotide as described herein, optionally and preferably in a serum sample. Use of known C reactive protein for prediction of increased risk of system failure or mortality is described with regard to PCT Application No. WO0196864, hereby incorporated by reference as if fully set forth herein. The PCT application describes a method comprising: a) adding one or more reagents to a test sample from a patient comprising at least part of a blood sample from the patient in order to cause formation of a complex comprising at least one acute phase protein and at least one human lipoprotein, while causing substantially no fibrin polymerization; b) measuring the formation of said complex over time so as to derive a time-dependent measurement profile; and c) determining a slope and/or total change in the time-dependent measurement profile so as to diagnose a condition of the patient. More preferably, wherein said at least one human lipoprotein comprises one or more of chylomicrons or remnants thereof, VLDL, IDL, LDL or HDL, and wherein said at least one acute phase protein comprises CRP.

Another non-limiting example of preferred utility is the detection, diagnosis and/or determination of haemostatic dysfunction in patients, which can lead to bleeding or thrombosis or particularly to Disseminated Intravascular Coagulation (DIC). The method comprises detecting a HSCREACT variant, for example a variant protein, protein fragment, peptide, polynucleotide, polynucleotide fragment and/or oligonucleotide as described herein, optionally and preferably in a serum sample. Use of known C reactive protein for prediction of haemostatic dysfunction in patients is described with regard to PCT Application No. WO0113125, hereby incorporated by reference as if fully set forth herein. The PCT application describes a method for detecting a precipitate in a test sample in the absence of clot formation. The precipitate detection allows for the prediction haemostatic dysfunction in patients. Preferably, the described method comprising: a) adding a reagent to a test sample comprising at least a component of a blood sample from a patient; b) measuring the formation of a precipitate due to the reaction of the test sample and the reagent, over time so as to derive a timedependent measurement profile, said reagent capable of forming a precipitate in the test sample without causing substantial fibrin polymerization. More preferably, a method for determining in a patient sample the presence of a complex of proteins comprising a C-reactive protein, comprising: a) obtaining a test sample from a patient; b) adding an alcohol, a clot inhibitor, and a metal cation; wherein a precipitate is formed which comprises a complex of proteins including C reactive protein.

Another non-limiting example of preferred utility is the differential diagnosis of diseases and conditions, that exhibit one or more similar or identical symptoms, such as dyspnea, fever, chest pain, abdominal pain, disturbances in metabolic state, neurologic dysfunction, hypertension, dizziness, and headache. The method comprises detecting a HSCREACT variant, for example a variant protein, protein fragment, peptide, polynucleotide, polynucleotide fragment and/or oligonucleotide as described herein, optionally and preferably in a serum sample. Use of known C reactive protein for differential diagnosis of diseases and conditions, that exhibit one or more similar or identical symptoms, such as dyspnea, fever, chest pain, abdominal pain, disturbances in metabolic state, neurologic dysfunction, hypertension, dizziness, and headache is described with regard to PCT Application No. WO2004059293, hereby incorporated by reference as if fully set forth herein. The PCT application describes a method for differential diagnosis of diseases and conditions, that exhibit one or more similar or identical symptoms, such as dyspnea, fever, chest pain, abdominal pain, disturbances in metabolic state, neurologic dysfunction, hypertension, dizziness, and headache.

The clinical presentation of certain diseases and conditions can often be strikingly similar, even though the underlying diseases, and the appropriate treatments to be given to one suffering from the various diseases, can be completely distinct. Differential diagnosis methods involve the comparison of symptoms and/or diagnostic test results known to be associated with one or more diseases that exhibit a similar clinical presentation to the symptoms and/or diagnostic results exhibited by the subject, in order to identify the underlying disease or condition present in the subject. In the case of dyspnea, the plurality of markers are preferably selected to rule in or out one or more, and preferably a plurality, of the following diagnoses: asthma, atrial fibrillation, chronic obstructive pulmonary disease (“COPD”), tracheal stenosis, obstructive endobroncheal tumor, pulmonary fibrosis, pneumoconiosis, lymphangitic carcinoniatosis, kyphoscoliosis, pleural effusion, amyotrophic lateral sclerosis, congestive heart failure, coronary artery disease, myocardial infarction, acute coronary syndrome, cardiomyopathy, valvular dysfunction, left ventricle hypertrophy, pericarditis, arrhythmia, pulmonary embolism, metabolic acidosis, chronic bronchitis, pneumonia, anxiety, sepsis, or aneunismic dissection. In a particularly preferred embodiment, the methods relate to defining the cause of dyspnea to rule in or rule out myocardial ischemia and cardiac necrosis, heart failure and pulmonary embolism. In yet another particularly preferred embodiment, the methods relate to defining the cause of dyspnea to rule in or rule out myocardial ischemia and cardiac necrosis, heart failure, pulmonary embolism and atrialfibrillation. The plurality of markers may also be used for prediction of risk that a subject may suffer from a future clinical outcome such as death or one or more nonfatal complications such as might require rehospitalization. In the case of abdominal pain, the plurality of markers are preferably selected to rule in or out a plurality of the following: aortic aneurysm, mesenteric embolism, pancreatitis, appendicitis, myocardial infarction, one or more infectious diseases described above, 5 influenza esophageal carcinoma, gastric adenocarcinoma, colorectal adenocarci 2 noma, pancreatic tumors including ductal adenocarcinoma, cystadenocarcinonia, and insulinoma. In the case of disturbances of metabolic state, the plurality of markers are preferably selected to rule in or out a plurality of the following: diabetes mellitus, diabetic ketoacidosis, alcoholic ketoacidosis, respiratory acidosis, respiratory alkalosis, nonketogenic hyperglycemia, hypoglycemia, renal failure, interstitial renal disease, COPD, pneumonia, pulmonary and edema, asthma. Such panels may diagnose one or more, and preferably distinguish between a plurality of, cardiovascular disorders selected from the group consisting of myocardial infarction, congestive heart failure, acute coronary syndrome, ST elevated ACS, non-ST elevated ACS, unstable angina, and/or pulmonary embolism; and/or predict risk that a subject may suffer from a future clinical outcome such as death, nonfatal myocardial infarction, recurrent ischemia requiring urgent revascularization, and/or recurrent ischemia requiring rehospitalization; and/or predict a risk of a future outcome in such diseases. Still other particularly preferred markers are those predictive of a subsequent cerebral vasospasm in patients presenting with subarachnoid hemorrhage, such as one or more markers related to blood pressure regulation, markers related to inflammation, markers related to apoptosis, and/or specific markers of neural tissue injury. In the case of neurologic dysfunction, the plurality of markers are preferably selected to rule in or out a plurality of the following: stroke, brain tumor, cerebral hypoxia, hypoglycemia, migraine, atrial fibrillation, myocardial infarctio'n, cardiac ischemia, peripheral vascular disease and seizure.

The WO2004059293 application preferably describes a method of symptom-based diagnosis of a subject, comprising: analyzing a test sample obtained from said subject for the presence or amount of a plurality of subject-derived markers, wherein said markers are selected to identify the presence or absence in said subject of a plurality of conditions within the differential diagnosis of a symptom exhibited by said subject; and correlating the presence or amount of said markers in said test sample to the presence or absence of each of said plurality of conditions. The WO2004059293 application further describes a method as above, wherein said symptom is selected from the group consisting of dyspnea, fever, chest pain, abdominal pain, disturbances in metabolic state, neurologic dysfunction, hypertension, dizziness, and headache. The WO2004059293 application further describes a method as above, wherein said symptom is dyspnea, and said plurality of conditions are selected from the group consisting of asthma, chronic obstructive pulmonary disease (“COPD”), tracheal stenosis, obstructive endobroncheal tumor, pulmonary fibrosis, pneumoconiosis, lymphangitic carcinomatosis, kyphoscoliosis, pleural effusion, amyotrophic lateral sclerosis, congestive heart failure, coronary artery disease, myocardial infarction, atrial fibrillation, cardiomyopathy, valvular dysfunction, left ventricle hypertrophy, pericarditis, arrhythmia, pulmonary embolism, metabolic acidosis, chronic bronchitis, pneumonia, anxiety, sepsis, and aneurismic dissection, myocardial infarction and pulmonary embolism, myocardial infarction and congestive heart failure, pulmonary embolism and congestive heart failure, or myocardial infarction, pulmonary embolism, and congestive heart failure. The WO2004059293 application further describes a method as above, wherein said symptom is chest pain, and said plurality of conditions are selected from the group consisting of stable angina, unstable angina, myocardial ischemia, cardiac necrosis, atrial fibrillation, myocardial infarction, musculoskeletal injury, cholecystitis, gastroesophageal reflux, pulmonary embolism, pericarditis, aortic dissection, pneumonia, anxiety, aortic dissection, myocardial ischemia, myocardial necrosis, myocardial infarction, and atrial fibrillation. The WO2004059293 application further describes a method as above, wherein said symptom is abdominal pain, and said plurality of conditions are selected from the group consisting of aortic dissection, mesenteric embolism, pancreatitis, appendicitis, myocardial ischemia, myocardial infarction, an infectious disease, influenza, esophageal carcinoma, gastric adenocarcinoma, colorectal adenocarcinoma, pancreatic ductal adenocarcinoma, cystadenocarcinoma, and insulinoma. The WO2004059293 application further describes a method as above, wherein said symptom is neurologic dysfunction, and said plurality of conditions are selected from the group consisting of stroke, ischemic stroke, subarachnoid hemorrhage, transient ischemic attack, intracerebral hemorrhage, hemorrhagic stroke, brain tumor, cerebral hypoxia, hypoglycemia, migraine, atrial fibrillation, myocardial infarction, cardiac ischemia, peripheral vascular disease, migraine, and seizure, ischemic stroke, hemorrhagic stroke, transient ischemic attack, atrial fibrillation, myocardial ischemia, and myocardial infarction.

Another non-limiting example of preferred utility is the detection, diagnosis and/or determination of multiple sclerosis, Alzheimer's disease and ischemia.

The method comprises detecting a HSCREACT variant, for example a variant protein, protein fragment, peptide, polynucleotide, polynucleotide fragment and/or oligonucleotide as described herein, optionally and preferably in a serum sample.

Use of known C reactive protein for diagnosing or monitoring various diseases including multiple sclerosis, Alzheimer's disease and ischemia is described with regard to PCT Application No. WO2004030522, hereby incorporated by reference as if fully set forth herein. The PCT application describes a method for diagnosis and monitoring of diseases and conditions by quantifying markers, including degradation products of disease-associated proteins, such as diketopiperazines composed of the two N-terminal amino acids or the two C-terminal amino acids of such proteins. The methods are useful for diagnosing or monitoring various diseases, including multiple sclerosis, rheumatoid arthritis, acute respiratory distress syndrome, cystic fibrosis, diabetes mellitus, Alzheimer's disease, Parkinson's disease, inflammation, ischemia, cerebral ischemia, placental ischemia, myocardial infarction, prostate cancer, pancreatitis, emphysema, renal disease, cancer, chemotherapy, hemoglobinopathies, anemnias or congestive heart failure.

Another non-limiting example of preferred utility is the detection, diagnosis, monitoring and/or determination of various conditions, including but not limited to bacterial infections, tumors associated with necrosis, acute myocardial infarction, acute inflammation phases of rheumatoid arthritis and transplant rejection, acute appendicitis, and inflammatory bowel disease and cardiovascular disease. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for diagnosing or monitoring various conditions including but not limited to bacterial infections, tumors associated with necrosis, acute myocardial infarction, acute inflammation phases of rheumatoid arthritis and transplant rejection, acute appendicitis, and inflammatory bowel disease and cardiovascular disease is described with regard to PCT Application No. WO2004025248, hereby incorporated by reference as if fully set forth herein.

C-reactive protein (CRP) is an acute phase reactant that is measured in order to assess the upper respiratory differential (URD) of patients. The concentration of CRP increases several-fold in response to different types of tissue damage and inflammation (Crockson et al, 1966). CRP is considered to be a prototypic acute phase reactant, synthesized in the liver as part of a coordinated response by hepatocytes to tissue injury or inflammation. Also, results from the 1997 Physicians Health Study have sparked interest in the utility of C-reactive protein (CRP) as being a significant disease indicator, particularly for males aged 40-84 (Luhr and Modi, 2000; Ridker et al, 1997). A rapid and accurate method for measuring CRP is needed to distinguish between allergic responses and viral and bacterial infections, and thereby to avoid unnecessary prescription of antibiotics.

The WO2004025248 application describes antibody pair screening methods for measuring CRP for rapid and accurate diagnosis and monitoring of various conditions including but not limited to bacterial infections, tumors associated with necrosis, acute myocardial infarction, acute inflammation phases of rheumatoid arthritis and transplant rejection, acute appendicitis, and inflammatory bowel disease and cardiovascular diseases.

Another non-limiting example of preferred utility is the early detection, diagnosis, monitoring and/or determination of sepsis. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for early detection of sepsis is described with regard to PCT Application No. WO03084388, hereby incorporated by reference as if fully set forth herein. The PCT describes A method of detecting early sepsis in a patient, wherein said method comprising the steps of. a) monitoring a plurality of biological markers over a period of time, b) independently deriving for each marker a marker statistic that is a statistical measure of extreme value of said marker over said period of time, and c) applying a decision rule to the marker statistics from step (b) to detect early sepsis in said patient.

Another non-limiting example of preferred utility is the early detection, diagnosis, monitoring and/or determination of host response to infection, systematic inflammatory response syndrome, or sepsis, involves correlating the lipoprotein measurement to an abnormality found in patients having severe infection. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for diagnosing and monitoring hemostatic dysfunction, severe infection and systematic inflammatory response syndrome is described with regard to PCT Application No. WO03073099, hereby incorporated by reference as if fully set forth herein. As described in WO 01/96864 (Dec. 20, 2001), a calcium-dependent complex between C reactive protein (CRP) and lipoprotein (particularly very low density lipoprotein (VLDL)) has been identified as the molecular mechanism underlying the biphasic waveform. The complex may be used to identify patients with sepsis, SIRS and septicaernia in addition to patients with other hemostatic dysfunction that can lead to bleeding or thrombosis including DIC. Further, WO 01/96864 describes detecting the complex by a clotting assay, latex agglutination or gold sol assay, and immunoassay whereby the precipitate is formed prior to or in the absence of clot formation, depending on the reagent used.

Another non-limiting example of preferred utility is for diagnostic assays for determining drug responsiveness. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for determining drug responsiveness is described with regard to PCT Application No. WO03062792, hereby incorporated by reference as if fully set forth herein. The PCT provides diagnostic assays for measuring the response to a drug or steroid by comparing mRNA levels expressed by a gene that is expected to respond to the drug to mRNA levels expressed by a gene that is not expected to respond to the drug. The assay also can be carried out at the protein level, by comparing the concentrations and/or activities (for example enzymatic activities) of the proteins corresponding to these mRNA species. The invention is based on observations that the expression of drug-sensitive genes, or changes in the expression of drug-sensitive genes, are useful as a marker for the cellular response to the drug or steroid. For example, the steroid responsive and non-responsive genes may be chosen depending upon their ability to respond to IL-1 and IL-6, administered separately or in combination, with or without steroids. For example, both the SA,41 gene and SA,42 genes respond to IL-1 strongly and IL-6 weakly, but only the responses of the SA,41 gene are augmented by glucocorticoid administration. By comparison, the C reactive protein gene (CRP) responds weakly to IL-1 and strongly to IL-6, and both responses are augmented by glucocorticoid administration. The SAA genes and the CRP gene therefore are markers for subsets of genes that respond differentially to certain cy-tokines alone or in combination with steroids and/or other drugs. Thus, a comparison of the RNA or protein levels of the SAA genes and the CRP genes, and/or other genes, may provide useful RNA or protein profiles which predict, determine, measure or monitor a subject's ability to respond to steroids at certain points during an acute phase response (i.e., depending upon the “Mix” of cytokines present at that point in time) or to certain cytokines, cytokine antagonists, antiinflammatory or other drug treatments in the absence of, or in combination with, endogenous or exogenous (i.e., therapeutically administered) steroids.

Another non-limiting example of preferred utility is for the detection, diagnosis and/or determination of disorders of iron metabolism, and subsequent recommendation for the treatment of these disorders. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for a method for detecting disorders of iron metabolism is described with regard to PCT Application No. WO03025583 and US application US20030232393, hereby incorporated by reference as if fully set forth herein. The PCT provides a method for detecting disorders of iron metabolism and in particular the differential diagnosis of disorders of iron metabolism by means of three independent parameters. The differential diagnosis can be used to classify disorders of iron metabolism and to recommend the required treatment and to monitor the progress and response to treatment. The PCT and the US applications provide a method for determining disorders of iron metabolism comprising the determination of: (i) a parameter which allows a determination of the total body iron stores, (ii) a parameter which allows a determination of at least one of the erythropoietic maturation process and its activity, and (iii) a parameter which allows a determination of unspecific disorders of iron metabolism. Wherein the parameter (i) in the method above is at least one of erythrocyte ferritin, zinc protoporphyrin, haemoglobin, myoglobin, transferrin, transferrin saturation, ferritin, haemosiderin, catalase, peroxidase and cytochrome; the parameter (ii) is at least one of erythrocyte indices, reticulocyte indices, FS-e (forward scatter erythrocytes) and soluble transferrin receptor (sTfR); and the parameter (iii) is at least one of acute phase proteins, regulators of acute phase protein synthesis and disorders of reticulocyte synthesis. C-reactive protein (CRP) is an examples of acute phase protein whose amount or concentration is determined in order to determine unspecific disorders of iron metabolism.

Another non-limiting example of preferred utility is for the detection, diagnosis and/or determination of stroke and cerebral injury. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for the diagnosis and evaluation of stroke and transient ischemic attacks is described with regard to PCT Application No. WO03016910, hereby incorporated by reference as if fully set forth herein. The PCT provides a method for the diagnosis and evaluation of stroke and transient ischemic attacks by, for example, by analyzing patient's samples for the presence or amount of a panel of markers comprising one or more specific markers for cerebral injury and one or more non-specific markers for cerebral injury. In various aspects, the invention provides methods for early detection and differentiation of stroke types and transient ischemic attacks, for determining the prognosis of a patient presenting with stroke symptoms, and identifying a patient at risk for cerebral vasospasm. According to the PCT, CRP may be useful in the identification of unstable angina and AMI when used with another marker that is specific for cardiac tissue injury.

Another non-limiting example of preferred utility is for the detection, diagnosis and/or determination of liver disfunction. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for the detection, diagnosis and/or determination of liver disfunction is described with regard to PCT Application No. WO03012450, hereby incorporated by reference as if fully set forth herein. The PCT provides a method for detecting and monitoring liver damage in a subject, by measuring a panel of components, including kallikrein-like peptidase, along with other blood enzymes and/or complement components. The liver damage panel members might include complement components O a, C4a, C I s and MASP-1, plasma prekallikrein and kallikrein/inhibitor (e.g. alpha macroglobulin and C I inhibitor) complexes, as well as serum amyloid protein (SAP) and/or C-reactive protein (CRP) representing acute phase proteins.

Another non-limiting example of preferred utility is for the detection, diagnosis and/or determination of deep venous thrombosis (DVT). The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for the detection, diagnosis and/or determination of deep venous thrombosis is described with regard to US patent Application No. US20040029286, hereby incorporated by reference as if fully set forth herein. The US application provides diagnostic method for patients with suspected deep venous thrombosis wherein by combined testing of patients' samples for D-dimer and CRP both the sensitivity and the negative predictive value of DVT diagnostics are increased up to 100%. A method for the diagnostic exclusion of the presence of deep venous thrombosis (DVT) in a patient at risk, comprising the following steps: (a) testing of a liquid sample from said patient with a reagent for the detection of D-dimer; (b) evaluation of the test result: (i) if the D-dimer concentration is found to be equal to or above a predefined cut-off value, DVT is not excluded with said patient; (ii) if the D-dimer concentration is found to be below the said cut-off value, further testing according to (c) is performed: (c) testing of a liquid sample from said patient with a reagent for the detection of C-reactive protein (CRP); (d) evaluation of the test result: if the CRP-concentration is found below a predefined cut-off value, DVT is excluded with said patient.

Another non-limiting example of preferred utility is for the detection, diagnosis and/or determination of arteriosclerosis. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for the detection, diagnosis and/or determination of arteriosclerosis is described with regard to US patent Application No. US20030077668, hereby incorporated by reference as if fully set forth herein. The US application provides a method for detecting LDL and denatured LDL (particularly, oxidized LDL) having a significant concerning with the onset and progress of arteriosclerosis and Alzheimer's disease, wherein a complex of denatured low density lipoprotein (particularly, oxidized LDL) with an acute phase reactant, blood coagulation fibrinolytic related protein or disinfectant substance produced by macrophage is used as a measuring subject. C-reactive protein (CRP) is an example of the acute phase reactant protein above.

Another non-limiting example of preferred utility is for assessing pentraxin-binding of particles for use in diagnosis of disease or abnormality, such as immune or autoimmune disorders, inflammatory disorders, sepsis, bacterial infection or cancer. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for assessing pentraxin-binding of particles for use in diagnosis of disease or abnormality, such as immune or autoimmune disorders, inflammatory disorders, sepsis, bacterial infection or cancer is described with regard to US patent Application No. US20030022245, hereby incorporated by reference as if fully set forth herein. The US application describes a method of assessing pentraxin-binding of particles for use in diagnosis of disease or abnormality, comprising: (a) exposing a biological test sample containing particles that comprise a pentraxin-binding receptor from a test subject to a ligand comprising a pentraxin in the presence of calcium; (b) determining quantitatively the level of binding between particles and ligand in said test sample; and (c) comparing the level of binding in said test sample to the level of binding in a control biological sample containing said particles from a healthy subject of the same species as the subject supplying the test sample, wherein a change in the level of binding in said test sample from that of the control sample is indicative of disease or abnormality, wherein the said pentraxin is C-reactive protein. The US application preferably describes the above method wherein an increase or decrease in the level of CRP bound to particles or the number of CRP-particles bound to cells, in said test sample over the same percentage or number in said control sample is indicative of an immune or auto-immune disorder. The US application more preferably describes the above method wherein said disorder is selected from the group consisting of diabetes, multiple sclerosis, Sjorgen's Syndrome rheumatoid arthritis, and systemic lupus erythematosis. The US application more preferably describes the above method wherein a decrease in the percentage of CRP-bound particles or the number of CRP-articles bound per cell, in said test sample over the said same percentage or number in said control sample is indicative of an inflammatory disorder or sepsis. The US application more preferably describes the above method wherein bound per cell, in said test sample over the same percentage or number in said control sample is indicative of cancer. The US application further describes the above method comprising examining the binding of said CRP in a fluorescent activated cell sorting assay and generating a light scatter region, wherein a decrease in the appearance of a distinct cluster of CRP-bound cells in a light scatter region equivalent to that of lymphocytes in said test sample of an infected mammal over the same percentage in an uninfected control is indicative of the presence of a bacterial infection.

Another non-limiting example of preferred utility is for the detection, diagnosis and/or determination of Bladder cancer. The method comprises detecting a HSCREACT variant, for example a variant protein, protein fragment, peptide, polynucleotide, polynucleotide fragment and/or oligonucleotide as described herein, optionally and preferably in a serum sample. A method for use of known C reactive protein for the detection, diagnosis and/or determination of Bladder cancer is described with regard to No. U.S. Pat. No. 4,447,545, hereby incorporated by reference as if fully set forth herein. The US patent discloses a method for screening populations to detect potential bladder cancer patients. The screening test is based on a discovered correlation between the respective ratios of C-reactive protein to total protein in urine and serum and the incidence of bladder cancer. The above method for screening a patient for the presence of a bladder carcinoma, comprising the steps of: (A) assaying for the ratio of C-reactive protein to total protein in a sample of the urine of said patient; (B) assaying for the ratio of C-reactive protein to total protein in a sample of the serum of said patient; and (C) comparing the ratio determined by assay in step A to the ratio determined by assay in step B, whereby (i) a finding of the absence of C-reactive protein in said urine sample is indicative of the absence of a bladder carcinoma; and (ii) a finding that the ratio determined in step A is greater than the ratio determined in step B is indicative of the presence of a bladder carcinoma.

Another non-limiting example of preferred utility is for the detection, diagnosis and/or determination of macular degeneration. The method comprises detecting a HSCREACT variant, for example a variant protein, protein fragment, peptide, polynucleotide, polynucleotide fragment and/or oligonucleotide as described herein, optionally and preferably in a serum sample. Macular degeneration is a clinical term that is used to describe a variety of diseases that are all characterized by a progressive loss of central vision associated with abnormalities of Bruch's membrane, the neural retina and the retinal pigment epithelium. A method for use of known C reactive protein for the detection, diagnosis and/or determination of macular degeneration is described with regard to PCT application No. WO0106262, hereby incorporated by reference as if fully set forth herein. The PCT discloses a method for diagnosing macular degeneration (MD), especially age-related macular degeneration (AMD), exudative or neovascular forms, by detecting a marker for arterial wall disruptive disorder, particularly abdominal aortic aneurysm. According to the above method of diagnosing macular degeneration, the said marker is for example, Complement reactive protein (CRP). Another example of use of known Complement reactive protein for for the detection, diagnosis and/or determination of macular degeneration is described with regard to PCT application No. WO0052479, and US patent application No: US20030149997, all hereby incorporated by reference as if fully set forth herein. The US application provides diagnostics, therapeutics and drug screening assays for arterial wall disruptive disorders, based on the discovery of a high level of correlation between the incidence of arterial wall disruptive disorders and the incidence of Age Related Macular Degeneration (AMD). In one embodiment, the arterial wall disruptive disorder is an aortic aneurysm. The above method for diagnosing, or determining a predisposition to developing, an arterial wall disruptive disorder in a subject, comprising detecting one or more genotypic or phenotypic markers for macular degeneration in the eye, wherein said marker is indicative of arterial wall disruptive disorder or of a predisposition to developing arterial wall disruptive disorder. The said drusen-associated marker is for example, complement reactive protein (CRP).

Another non-limiting example of preferred utility is for early diagnosis of premalignant lesions or determining prognosis of malignant lesions. The method comprises detecting a HSCREACT variant, for example a variant protein, protein fragment, peptide, polynucleotide, polynucleotide fragment and/or oligonucleotide as described herein, optionally and preferably in a serum sample. A method for use of known Complement reactive protein for early diagnosis of premalignant lesions or determining prognosis of malignant lesions is described with regard to US patent application No. US20030129677, hereby incorporated by reference as if fully set forth herein. The US application discloses a method for early diagnosis of premalignant lesions or determining prognosis of malignant lesions in patients by measuring quantity of complement regulatory protein-anti-CRP antibody complex as compared to normal level, in a sample.

Another non-limiting example of preferred utility is for the early detection of pregnancy failure, spontaneous abortion or premature birth. The method comprises detecting a HSCREACT variant, for example a variant protein, protein fragment, peptide, polynucleotide, polynucleotide fragment and/or oligonucleotide as described herein, optionally and preferably in a serum sample. A method for use of known Complement reactive protein for the early detection of pregnancy failure, spontaneous abortion or premature birth is described with regard to US patent application No. US20030129674, hereby incorporated by reference as if fully set forth herein. The US application discloses a method for diagnosing a predisposition for pregnancy failure, spontaneous abortion or premature birth in a pregnant patient comprising: (a) contacting a physiological fluid potentially comprising a cell membrane-associated complement regulatory protein (CRP) from the patient with a anti-CRP antibody to form an CRP-antibody complex; and (b) measuring the quantity of CRP-antibody complex in the physiological fluid as compared to a normal control level, wherein the quantity of CRP-antibody complex as compared to the normal control is indicative for a predisposition for pregnancy failure, spontaneous abortion or premature birth.

Cluster HSCREACT belongs to a family of proteins which are known to have functions related to neuronal associated disorders, including but not limited to, APCS (Amyloid P), NPTX1, NPTXR, NPTX2, PTX3, PAPPA & PAPPA2. These functions are described below; one or more variants of cluster HSCREACT may optionally have one or more diagnostic utilities related to these functions.

APCS (Amyloid P) deposit, and radiolabeled SAP is a specific, sensitive, quantitative diagnostic tracer for systemic amyloid deposits in vivo (Lovat L B, et al. Scintigraphy with 123]-serum amyloid P component in Alzheimer disease. Alzheimer Dis Assoc Disord. 1998 September; 12(3):208-10; Hawkins P N, Myers M J, Lavender J P, et al. Diagnostic radionuclide imaging of amyloid: biological targeting by circulating human serum amyloid P component. Lancet 1988; i:1413-1418).

NPTX1 is overexpressed in Szchizophrenia (Gabor et al. Nature Biotech. 22 (5) 615-621-2004). Based on its function and expression profile, NPTX1 could play a role in the development of GTS (Tourette and OCD): (Zhang, H et al. Genomewide scan of hoarding in sib pairs in which both sibs have Gilles de la Tourette syndrome. Am. J. Hum. Genet. 70: 896-904, 2002.). NPTX1 is a among a group of Neuronal-associated genes in melanoma (Seftor et al. Expression of multiple molecular phenotypes by aggressive melanoma tumor cells: role in vasculogenic mimicry Critical Reviews in Oncology/Hematology 000 (2001) 000-000 (uncorrected proof));

All of these functions may optionally be diagnostic utilities of one or more HSCREACT variants according to the present invention.

Table 99 below describes diagnostic utilities for the cluster HSCREACT that were found through microarrays, including the statistical significance thereof and a reference. One or more HSCREACT variants according to the present invention may optionally have one or more of these utilities.

TABLE 99

| | Statistical | |
|---|---|---|
| Diagnostic utility | significance | reference |
| Gene over expressed | 0.003 | 1. Segal NH, Houghton AN. J |
| in melanoma (vs. Soft | | Clin Oncol (2003) Classification |
| Tissue Sarcoma) | | of clear-cell sarcoma as a |
| | | subtype of melanoma by |
| | | genomic profiling. |

Other non-limiting exemplary utilities for HSACMHCP variants according to the present invention are described in greater detail below and also with regard to the previous section on clinical utility.

As noted above, cluster HSCREACT features 10 transcript(s), which were listed in Table 94 above. These transcript(s) encode for protein(s) which are variant(s) of protein C-reactive protein precursor (SEQ ID NO:316). A description of each variant protein according to the present invention is now provided.

Variant protein HSCREACT_PEA_{—}1_P9 (SEQ ID NO:317) according to the present invention has an amino acid sequence; it is encoded by transcript(s) HSCREACT_PEA_{—}1_T12 (SEQ ID NO:251). An alignment is given to the known protein (C-reactive protein precursor (SEQ ID NO:316)) at the end of the application. One or more alignments to one or more previously published protein sequences are given in the alignment table located on the attached CDROM. A brief description of the relationship of the variant protein according to the present invention to each such aligned protein is as follows:

Comparison report between HSCREACT_PEA_{—}1_P9 (SEQ ID NO:317) and CRP_HUMAN (SEQ ID NO:316):

1. An isolated chimeric polypeptide encoding for HSCREACT_PEA_{—}1_P9 (SEQ ID NO:317), comprising a first amino acid sequence being at least 90% homologous to MEKLLCFLVLTSLSHAFGQTDMSRKAFVFPKESDTSYVSLKAPLTKPLKAFTVCLHFYTELSST corresponding to amino acids 1-64 of CRP_HUMAN (SEQ ID NO:316), which also corresponds to amino acids 1-64 of HSCREACT_PEA_{—}1_P9 (SEQ ID NO:317), a second amino acid sequence bridging amino acid sequence comprising of H, and a third amino acid sequence being at least 90% homologous to EINTIYLGGPFSPNVLNWRALKYEVQGEVFTKPQLWP corresponding to amino acids 188-224 of CRP_HUMAN (SEQ ID NO:316), which also corresponds to amino acids 66-102 of HSCREACT_PEA_{—}1_P9 (SEQ ID NO:317), wherein said first amino acid sequence, second amino acid sequence and third amino acid sequence are contiguous and in a sequential order.

2. An isolated polypeptide encoding for an edge portion of HSCREACT_PEA_{—}1_P9 (SEQ ID NO:317), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise

THE having a structure as follows (numbering according to HSCREACT_PEA_{—}1_P9 (SEQ ID NO:317)): a sequence starting from any of amino acid numbers 64−x to 64; and ending at any of amino acid numbers 66+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The phosphorylation sites of variant protein HSCREACT_PEA_{—}1_P9 (SEQ ID NO:317), as compared to the known protein C-reactive protein precursor (SEQ ID NO:316), are described in Table 100 (given according to their position(s) on the amino acid sequence in the first column; the second column indicates whether the phosphorylation site is present in the variant protein; and the last column indicates whether the position is different on the variant protein).

TABLE 100
Phosphorylation site(s)

| Position(s) on known | Present in | Position in |
|---|---|---|
| amino acid sequence | variant protein? | variant protein? |
| 19 | yes | 19 |

The variant protein has the following domains, as determined by using InterPro. The domains are described in Table 101:

TABLE 101
InterPro domain(s)

| | Domain | | Position(s) |
|---|---|---|---|
| InterPro ID | description | Analysis type | on protein |
| IPR001759 | Pentaxin | HMMPfam | 25-64, 66-98 |
| IPR001759 | Pentaxin | HMMSmart | 19-102 |
| IPR001759 | Pentaxin | BlastProDom | 19-64, 79-102 |

Variant protein HSCREACT_PEA_{—}1_P9 (SEQ ID NO:317) is encoded by the following transcript(s): HSCREACT_PEA_{—}1_T12 (SEQ ID NO:251). The coding portion of transcript HSCREACT_PEA_{—}1_T12 (SEQ ID NO:251) starts at position 117 and ends at position 422. The transcript also has the following SNPs as listed in Table 102 (given according to their position on the nucleotide sequence, with the alternative nucleic acid listed; the last column indicates whether the SNP is known or not; the presence of known SNPs in variant protein HSCREACT_PEA_{—}1_P9 (SEQ ID NO:317) sequence provides support for the deduced sequence of this variant protein according to the present invention).

TABLE 102
Nucleic acid SNPs

| SNP position on | Alternative | Previously |
|---|---|---|
| nucleotide sequence | nucleic acid | known SNP? |
| 11 | G -> C | Yes |
| 11 | G -> T | Yes |
| 90 | T -> | No |
| 90 | T -> C | No |
| 129 | T -> | No |
| 164 | T -> | No |
| 194 | T -> | No |
| 317 | T -> C | No |
| 420 | C -> | No |
| 420 | C -> G | No |
| 527 | C -> T | Yes |
| 641 | C -> A | Yes |
| 649 | C -> T | Yes |
| 891 | C -> A | Yes |
| 893 | C -> T | No |
| 1139 | T -> | No |
| 1212 | T -> C | Yes |
| 1233 | C -> T | Yes |
| 1310 | T -> C | Yes |
| 1315 | C -> T | Yes |
| 1318 | C -> T | Yes |
| 1334 | C -> T | Yes |
| 1426 | C -> T | Yes |
| 1498 | A -> T | Yes |
| 1507 | G -> A | Yes |

Variant protein HSCREACT_PEA_{—}1_P10 (SEQ ID NO:318) according to the present invention has an amino acid sequence; it is encoded by transcript(s) HSCREACT_PEA_{—}1_T3 (SEQ ID NO:252). An alignment is given to the known protein (C-reactive protein precursor (SEQ ID NO:316)) at the end of the application. One or more alignments to one or more previously published protein sequences are given in the alignment table located on the attached CDROM. A brief description of the relationship of the variant protein according to the present invention to each such aligned protein is as follows:

Comparison report between HSCREACT_PEA_{—}1_P10 (SEQ ID NO:318) and CRP_HUMAN (SEQ ID NO:316):

1. An isolated chimeric polypeptide encoding for HSCREACT_PEA_{—}1_P10 (SEQ ID NO:318), comprising a first amino acid sequence being at least 90% homologous to MEKLLCFLVLTSLSHAFGQTDMSRKAFVFPKESDTSYVSLKAPLTKPLKAFTVCLHFYTELSSTRG corresponding to amino acids 1-66 of CRP_HUMAN (SEQ ID NO:316), which also corresponds to amino acids 1-66 of HSCREACT_PEA_{—}1_P10 (SEQ ID NO:318).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The phosphorylation sites of variant protein HSCREACT_PEA_{—}1_P10 (SEQ ID NO:318), as compared to the known protein C-reactive protein precursor (SEQ ID NO:316), are described in Table 103 (given according to their position(s) on the amino acid sequence in the first column; the second column indicates whether the phosphorylation site is present in the variant protein; and the last column indicates whether the position is different on the variant protein).

TABLE 103
Phosphorylation site(s)

| Position(s) on known | Present in | Position in |
|---|---|---|
| amino acid sequence | variant protein? | variant protein? |
| 19 | yes | 19 |

The variant protein has the following domains, as determined by using InterPro. The domains are described in Table 104:

TABLE 104
InterPro domain(s)

| | Domain | | Position(s) |
|---|---|---|---|
| InterPro ID | description | Analysis type | on protein |
| IPR001759 | Pentaxin | HMMPfam | 25-66 |
| IPR001759 | Pentaxin | BlastProDom | 19-66 |

Variant protein HSCREACT_PEA_{—}1_P10 (SEQ ID NO:318) is encoded by the following transcript(s): HSCREACT_PEA_{—}1_T13 (SEQ ID NO:252). The coding portion of transcript HSCREACT_PEA_{—}1_T13 (SEQ ID NO:252) starts at position 117 and ends at position 314. The transcript also has the following SNPs as listed in Table 105 (given according to their position on the nucleotide sequence, with the alternative nucleic acid listed; the last column indicates whether the SNP is known or not; the presence of known SNPs in variant protein HSCREACT_PEA_{—}1_P10 (SEQ ID NO:318) sequence provides support for the deduced sequence of this variant protein according to the present invention).

TABLE 105
Nucleic acid SNPs

| SNP position on nucleotide | Alternative | Previously |
|---|---|---|
| sequence | nucleic acid | known SNP? |
| 11 | G -> C | Yes |
| 11 | G -> T | Yes |
| 90 | T -> | No |
| 90 | T -> C | No |
| 129 | T -> | No |
| 164 | T -> | No |
| 194 | T -> | No |
| 321 | T -> C | No |
| 424 | C -> | No |
| 424 | C -> G | No |
| 531 | C -> T | Yes |
| 645 | C -> A | Yes |
| 653 | C -> T | Yes |
| 895 | C -> A | Yes |
| 897 | C -> T | No |
| 1143 | T -> | No |
| 1216 | T -> C | Yes |
| 1237 | C -> T | Yes |
| 1314 | T -> C | Yes |
| 1319 | C -> T | Yes |
| 1322 | C -> T | Yes |
| 1338 | C -> T | Yes |
| 1430 | C -> T | Yes |
| 1502 | A -> T | Yes |
| 1511 | G -> A | Yes |

Variant protein HSCREACT_PEA_{—}1_P12 (SEQ ID NO:319) according to the present invention has an amino acid sequence; it is encoded by transcript(s) HSCREACT_PEA_{—}1_T15 (SEQ ID NO:253). An alignment is given to the known protein (C-reactive protein precursor (SEQ ID NO:316)) at the end of the application. One or more alignments to one or more previously published protein sequences are given in the alignment table located on the attached CDROM. A brief description of the relationship of the variant protein according to the present invention to each such aligned protein is as follows:

Comparison report between HSCREACT_PEA_{—}1_P12 (SEQ ID NO:319) and CRP_HUMAN (SEQ ID NO:316):

1. An isolated chimeric polypeptide encoding for HSCREACT_PEA_{—}1_P12 (SEQ ID NO:319), comprising a first amino acid sequence being at least 90% homologous to MEKLLCFLVLTSLSHAFGQTDMSRKAFVFPKESDTSYVSLKAPLTKPLKAFTVCLHFYTELSSTRG corresponding to amino acids 1-66 of CRP_HUMAN (SEQ ID NO:316), which also corresponds to amino acids 1-66 of HSCREACT_PEA_{—}1_P12 (SEQ ID NO:319), and a second amino acid sequence being at least 90% homologous to PNVLNWRALKYEVQGEVFTKPQLWP corresponding to amino acids 200-224 of CRP_HUMAN (SEQ ID NO:316), which also corresponds to amino acids 67-91 of HSCREACT_PEA_{—}1_P12 (SEQ ID NO:319), wherein said first amino acid sequence and second amino acid sequence are contiguous and in a sequential order.

2. An isolated chimeric polypeptide encoding for an edge portion of HSCREACT_PEA_{—}1_P12 (SEQ ID NO:319), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GP, having a structure as follows: a sequence starting from any of amino acid numbers 66−x to 66; and ending at any of amino acid numbers 67+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The phosphorylation sites of variant protein HSCREACT_PEA_{—}1_P12 (SEQ ID NO:319), as compared to the known protein C-reactive protein precursor (SEQ ID NO:316), are described in Table 106 (given according to their position(s) on the amino acid sequence in the first column; the second column indicates whether the phosphorylation site is present in the variant protein; and the last column indicates whether the position is different on the variant protein).

TABLE 106
Phosphorylation site(s)

| Position(s) on known | Present in | Position in |
|---|---|---|
| amino acid sequence | variant protein? | variant protein? |
| 19 | yes | 19 |

The variant protein has the following domains, as determined by using InterPro. The domains are described in Table 107:

TABLE 107
InterPro domain(s)

| | Domain | | Position(s) |
|---|---|---|---|
| InterPro ID | description | Analysis type | on protein |
| IPR001759 | Pentaxin | HMMPfam | 25-66, 67-87 |
| IPR001759 | Pentaxin | HMMSmart | 19-91 |
| IPR001759 | Pentaxin | BlastProDom | 19-84 |

Variant protein HSCREACT_PEA_{—}1_P12 (SEQ ID NO:319) is encoded by the following transcript(s): HSCREACT_PEA_{—}1_T5 (SEQ ID NO:253). The coding portion of transcript HSCREACT_PEA_{—}1_T15 (SEQ ID NO:253) starts at position 117 and ends at position 389. The transcript also has the following SNPs as listed in Table 108 (given according to their position on the nucleotide sequence, with the alternative nucleic acid listed; the last column indicates whether the SNP is known or not; the presence of known SNPs in variant protein HSCREACT_PEA_{—}1_P12 (SEQ ID NO:319) sequence provides support for the deduced sequence of this variant protein according to the present invention).

TABLE 108
Nucleic acid SNPs

| SNP position on | Alternative | Previously |
|---|---|---|
| nucleotide sequence | nucleic acid | known SNP? |
| 11 | G -> C | Yes |
| 11 | G -> T | Yes |
| 90 | T -> | No |
| 90 | T -> C | No |
| 129 | T -> | No |
| 164 | T -> | No |
| 194 | T -> | No |
| 387 | C -> | No |
| 387 | C -> G | No |
| 494 | C -> T | Yes |
| 608 | C -> A | Yes |
| 616 | C -> T | Yes |
| 858 | C -> A | Yes |
| 860 | C -> T | No |
| 1106 | T -> | No |
| 1179 | T -> C | Yes |
| 1200 | C -> T | Yes |
| 1277 | T -> C | Yes |
| 1282 | C -> T | Yes |
| 1285 | C -> T | Yes |
| 1301 | C -> T | Yes |
| 1393 | C -> T | Yes |
| 1465 | A -> T | Yes |
| 1474 | G -> A | Yes |

Variant protein HSCREACT_PEA_{—}1_P16 (SEQ ID NO:320) according to the present invention has an amino acid sequence; it is encoded by transcript(s) HSCREACT_PEA_{—}1_T22 (SEQ ID NO:254). An alignment is given to the known protein (C-reactive protein precursor (SEQ ID NO:316)) at the end of the application. One or more alignments to one or more previously published protein sequences are given in the alignment table located on the attached CDROM. A brief description of the relationship of the variant protein according to the present invention to each such aligned protein is as follows:

Comparison report between HSCREACT_PEA_{—}1_P16 (SEQ ID NO:320) and CRP_HUMAN (SEQ ID NO:316):

1. An isolated chimeric polypeptide encoding for HSCREACT_PEA_{—}1_P16 (SEQ ID NO:320), comprising a first amino acid sequence being at least 90% homologous to MEKLLCFLVLTSLSHAFGQTDMSRKAFVFPKESDTSYVSLKAPLTKPLKAFTVCLHFYTELSSTRG YSIFSYATKRQDNEILIFWSKDIGYSFTVGGSEILFEVPEVTVAPVHICTSWESASGIVEFWVDGKPR VRKSLKKGYTVGAEASIILGQEQDSF corresponding to amino acids 1-160 of CRP_HUMAN (SEQ ID NO:316), which also corresponds to amino acids 1-160 of HSCREACT_PEA_{—}1_P16 (SEQ ID NO:320), and a second amino acid sequence being at least 70%, optionally at least 80%, preferably at least 85%, more preferably at least 90% and most preferably at least 95% homologous to a polypeptide having the sequence VSESGHWPGVWFGSRVLIIMS (SEQ ID NO: 650) corresponding to amino acids 161-181 of HSCREACT_PEA_{—}1_P16 (SEQ ID NO:320), wherein said first amino acid sequence and second amino acid sequence are contiguous and in a sequential order.

2. An isolated polypeptide encoding for a tail of HSCREACT_PEA_{—}1_P16 (SEQ ID NO:320), comprising a polypeptide being at least 70%, optionally at least about 80%, preferably at least about 85%, more preferably at least about 90% and most preferably at least about 95% homologous to the sequence VSESGHWPGVWFGSRVLIIMS (SEQ ID NO: 650) in HSCREACT_PEA_{—}1_P16 (SEQ ID NO:320).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The phosphorylation sites of variant protein HSCREACT_PEA_{—}1_P16 (SEQ ID NO:320), as compared to the known protein C-reactive protein precursor (SEQ ID NO:316), are described in Table 109 (given according to their position(s) on the amino acid sequence in the first column; the second column indicates whether the phosphorylation site is present in the variant protein; and the last column indicates whether the position is different on the variant protein).

TABLE 109
Phosphorylation site(s)

| Position(s) on known | Present in | Position in |
|---|---|---|
| amino acid sequence | variant protein? | variant protein? |
| 19 | yes | 19 |

The variant protein has the following domains, as determined by using InterPro. The domains are described in Table 110:

TABLE 110
InterPro domain(s)

| | Domain | | |
|---|---|---|---|
| InterPro ID | description | Analysis type | Position(s) on protein |
| IPR001759 | Pentaxin | FPrintScan | 113-131, 139-158, |
| | | | 48-62, 70-84 |
| IPR001759 | Pentaxin | HMMPfam | 25-160 |
| IPR001759 | Pentaxin | HMMSmart | 19-178 |
| IPR001759 | Pentaxin | ScanRegExp | 113-120 |
| IPR001759 | Pentaxin | BlastProDom | 19-160 |

Variant protein HSCREACT_PEA_{—}1_P16 (SEQ ID NO:320) is encoded by the following transcript(s): HSCREACT_PEA_{—}1_T22 (SEQ ID NO:254). The coding portion of transcript HSCREACT_PEA_{—}1_T22 (SEQ ID NO:254) starts at position 117 and ends at position 659. The transcript also has the following SNPs as listed in Table 111 (given according to their position on the nucleotide sequence, with the alternative nucleic acid listed; the last column indicates whether the SNP is known or not; the presence of known SNPs in variant protein HSCREACT_PEA_{—}1_P16 (SEQ ID NO:320) sequence provides support for the deduced sequence of this variant protein according to the present invention).

TABLE 111
Nucleic acid SNPs

| SNP position on | Alternative | Previously |
|---|---|---|
| nucleotide sequence | nucleic acid | known SNP? |
| 11 | G -> C | Yes |
| 11 | G -> T | Yes |
| 90 | T -> | No |
| 90 | T -> C | No |
| 129 | T -> | No |
| 164 | T -> | No |
| 194 | T -> | No |
| 336 | A -> G | No |
| 366 | T -> | No |
| 517 | G -> | No |
| 599 | C -> A | Yes |
| 601 | C -> T | No |
| 847 | T -> | No |
| 920 | T -> C | Yes |
| 941 | C -> T | Yes |
| 1018 | T -> C | Yes |
| 1023 | C -> T | Yes |
| 1026 | C -> T | Yes |
| 1042 | C -> T | Yes |
| 1134 | C -> T | Yes |
| 1206 | A -> T | Yes |
| 1215 | G -> A | Yes |

Variant protein HSCREACT_PEA_{—}1_P22 (SEQ ID NO:321) according to the present invention has an amino acid sequence; it is encoded by transcript(s) HSCREACT_PEA_{—}1_T29 (SEQ ID NO:255). An alignment is given to the known protein (C-reactive protein precursor (SEQ ID NO:316)) at the end of the application. One or more alignments to one or more previously published protein sequences are given in the alignment table located on the attached CDROM. A brief description of the relationship of the variant protein according to the present invention to each such aligned protein is as follows:

Comparison report between HSCREACT_PEA_{—}1_P22 (SEQ ID NO:321) and CRP_HUMAN (SEQ ID NO:316):

1. An isolated chimeric polypeptide encoding for HSCREACT_PEA_{—}1_P22 (SEQ ID NO:321), comprising a first amino acid sequence being at least 90% homologous to MEKLLCFLVLTSLSHAFGQTDMSRKAFVFPKESDTSYVSLKAPLTKPLKAFTVCLHFYTELSSTRG corresponding to amino acids 1-66 of CRP_HUMAN (SEQ ID NO:316), which also corresponds to amino acids 1-66 of HSCREACT_PEA_{—}1_P22 (SEQ ID NO:321), and a second amino acid sequence being at least 70%, optionally at least 80%, preferably at least 85%, more preferably at least 90% and most preferably at least 95% homologous to a polypeptide having the sequence AFLILWLFWETPPLFHTNLVGL (SEQ ID NO: 651) corresponding to amino acids 67-88 of HSCREACT_PEA_{—}1_P22 (SEQ ID NO:321), wherein said first amino acid sequence and second amino acid sequence are contiguous and in a sequential order.

2. An isolated polypeptide encoding for a tail of HSCREACT_PEA_{—}1_P22 (SEQ ID NO:321), comprising a polypeptide being at least 70%, optionally at least about 80%, preferably at least about 85%, more preferably at least about 90% and most preferably at least about 95% homologous to the sequence AFLILWLFWETPPLFHTNLVGL (SEQ ID NO: 651) in HSCREACT_PEA_{—}1_P22 (SEQ ID NO:321).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The phosphorylation sites of variant protein HSCREACT_PEA_{—}1_P22 (SEQ ID NO:321), as compared to the known protein C-reactive protein precursor (SEQ ID NO:316), are described in Table 112 (given according to their position(s) on the amino acid sequence in the first column; the second column indicates whether the phosphorylation site is present in the variant protein; and the last column indicates whether the position is different on the variant protein).

TABLE 112
Phosphorylation site(s)

| Position(s) on known amino | Present in | |
|---|---|---|
| acid sequence | variant protein? | Position in variant protein? |
| 19 | yes | 19 |

The variant protein has the following domains, as determined by using InterPro. The domains are described in Table 113:

TABLE 113
InterPro domain(s)

| | Domain | | |
|---|---|---|---|
| InterPro ID | description | Analysis type | Position(s) on protein |
| IPR001759 | Pentaxin | HMMPfam | 25-66 |
| IPR001759 | Pentaxin | BlastProDom | 19-66 |

Variant protein HSCREACT_PEA_{—}1_P22 (SEQ ID NO:321) is encoded by the following transcript(s): HSCREACT_PEA_{—}1_T29 (SEQ ID NO:255). The coding portion of transcript HSCREACT_PEA_{—}1_T29 (SEQ ID NO:255) starts at position 117 and ends at position 380. The transcript also has the following SNPs as listed in Table 114 (given according to their position on the nucleotide sequence, with the alternative nucleic acid listed; the last column indicates whether the SNP is known or not; the presence of known SNPs in variant protein HSCREACT_PEA_{—}1_P22 (SEQ ID NO:321) sequence provides support for the deduced sequence of this variant protein according to the present invention).

TABLE 114
Nucleic acid SNPs

| SNP position on nucleotide | Alternative | |
|---|---|---|
| sequence | nucleic acid | Previously known SNP? |
| 11 | G -> C | Yes |
| 11 | G -> T | Yes |
| 90 | T -> | No |
| 90 | T -> C | No |
| 129 | T -> | No |
| 164 | T -> | No |
| 194 | T -> | No |
| 370 | T -> C | Yes |
| 391 | C -> T | Yes |
| 468 | T -> C | Yes |
| 473 | C -> T | Yes |
| 476 | C -> T | Yes |
| 492 | C -> T | Yes |
| 584 | C -> T | Yes |
| 656 | A -> T | Yes |
| 665 | G -> A | Yes |

variant protein HSCREACT_PEA_{—}1_P2 (SEQ ID NO:322) according to the present invention has an amino acid sequence; it is encoded by transcript(s) HSCREACT_PEA_{—}1_T33 (SEQ ID NO:258). An alignment is given to the known protein (C-reactive protein precursor (SEQ ID NO:316)) at the end of the application. One or more alignments to one or more previously published protein sequences are given in the alignment table located on the attached CDROM. A brief description of the relationship of the variant protein according to the present invention to each such aligned protein is as follows:

Comparison report between HSCREACT_PEA_{—}1_P28 (SEQ ID NO:322) and CRP_HUMAN (SEQ ID NO:316):

1. An isolated chimeric polypeptide encoding for HSCREACT_PEA_{—}1_P28 (SEQ ID NO:322), comprising a first amino acid sequence being at least 90% homologous to MEKLLCFLVLTSLSHAFGQTDMSRKAFVFPKESDTSYVSLKAPLTKPLKAFTVCLHFYTELSST corresponding to amino acids 1-64 of CRP_HUMAN (SEQ ID NO:316), which also corresponds to amino acids 1-64 of HSCREACT_PEA_{—}1_P28 (SEQ ID NO:322), and a second amino acid sequence being at least 70%, optionally at least 80%, preferably at least 85%, more preferably at least 90% and most preferably at least 95% homologous to a polypeptide having the sequence LLS corresponding to amino acids 65-67 of HSCREACT_PEA_{—}1_P28 (SEQ ID NO:322), wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The phosphorylation sites of variant protein HSCREACT_PEA_{—}1_P28 (SEQ ID NO:322), as compared to the known protein C-reactive protein precursor (SEQ ID NO:316), are described in Table 115 (given according to their position(s) on the amino acid sequence in the first column; the second column indicates whether the phosphorylation site is present in the variant protein; and the last column indicates whether the position is different on the variant protein).

TABLE 115
Phosphorylation site(s)

| Position(s) on known amino | Present in | |
|---|---|---|
| acid sequence | variant protein? | Position in variant protein? |
| 19 | yes | 19 |

The variant protein has the following domains, as determined by using InterPro. The domains are described in Table 116:

TABLE 116
InterPro domain(s)

| | Domain | | |
|---|---|---|---|
| InterPro ID | description | Analysis type | Position(s) on protein |
| IPR001759 | Pentaxin | HMMPfam | 25-64 |
| IPR001759 | Pentaxin | BlastProDom | 19-64 |

Variant protein HSCREACT_PEA_{—}1_P28 (SEQ ID NO:322) is encoded by the following transcript(s): HSCREACT_PEA_{—}1_T33 (SEQ ID NO:258). The coding portion of transcript HSCREACT_PEA_{—}1_T33 (SEQ ID NO:258) starts at position 117 and ends at position 317. The transcript also has the following SNPs as listed in Table 117 (given according to their position on the nucleotide sequence, with the alternative nucleic acid listed; the last column indicates whether the SNP is known or not; the presence of known SNPs in variant protein HSCREACT_PEA_{—}1_P28 (SEQ ID NO:322) sequence provides support for the deduced sequence of this variant protein according to the present invention).

TABLE 117
Nucleic acid SNPs

| SNP position on nucleotide | Alternative | |
|---|---|---|
| sequence | nucleic acid | Previously known SNP? |
| 11 | G -> C | Yes |
| 11 | G -> T | Yes |
| 90 | T -> | No |
| 90 | T -> C | No |
| 129 | T -> | No |
| 164 | T -> | No |
| 194 | T -> | No |
| 366 | T -> C | Yes |
| 387 | C -> T | Yes |
| 464 | T -> C | Yes |
| 469 | C -> T | Yes |
| 472 | C -> T | Yes |
| 488 | C -> T | Yes |
| 580 | C -> T | Yes |
| 652 | A -> T | Yes |
| 661 | G -> A | Yes |

Table 118 below describes the starting and ending position of HSCREACT_PEA_{—}1_node_{—}8 (SEQ ID NO:266) on each of the relevant transcripts. Experimental results for this segment are described below.

TABLE 118
Segment location on transcripts

| | Segment | Segment |
|---|---|---|
| | starting | ending |
| Transcript name | position | position |
| HSCREACT_PEA_1_T12 (SEQ ID NO: 251) | 178 | 250 |
| HSCREACT_PEA_1_T13 (SEQ ID NO: 252) | 178 | 250 |
| HSCREACT_PEA_1_T15 (SEQ ID NO: 253) | 178 | 250 |
| HSCREACT_PEA_1_T22 (SEQ ID NO: 254) | 178 | 250 |
| HSCREACT_PEA_1_T29 (SEQ ID NO: 255) | 178 | 250 |
| HSCREACT_PEA_1_T30 (SEQ ID NO: 256) | 178 | 250 |
| HSCREACT_PEA_1_T32 (SEQ ID NO: 257) | 178 | 250 |
| HSCREACT_PEA_1_T33 (SEQ ID NO: 258) | 178 | 250 |
| HSCREACT_PEA_1_T38 (SEQ ID NO: 259) | 178 | 250 |
| HSCREACT_PEA_1_T39 (SEQ ID NO: 260) | 178 | 250 |

Table 119 below describes the starting and ending position of HSCREACT_PEA_{—}1_node_{—}11 (SEQ ID NO:269) on each of the relevant transcripts. Experimental results for this segment are described below.

TABLE 119
Segment location on transcripts

| | Segment | Segment |
|---|---|---|
| | starting | ending |
| Transcript name | position | position |
| HSCREACT_PEA_1_T12 (SEQ ID NO: 251) | 284 | 309 |
| HSCREACT_PEA_1_T13 (SEQ ID NO: 252) | 284 | 309 |
| HSCREACT_PEA_1_T15 (SEQ ID NO: 253) | 284 | 309 |
| HSCREACT_PEA_1_T22 (SEQ ID NO: 254) | 284 | 309 |
| HSCREACT_PEA_1_T29 (SEQ ID NO: 255) | 284 | 309 |
| HSCREACT_PEA_1_T30 (SEQ ID NO: 256) | 284 | 309 |
| HSCREACT_PEA_1_T32 (SEQ ID NO: 257) | 284 | 309 |
| HSCREACT_PEA_1_T33 (SEQ ID NO: 258) | 284 | 309 |
| HSCREACT_PEA_1_T38 (SEQ ID NO: 259) | 284 | 309 |
| HSCREACT_PEA_1_T39 (SEQ ID NO: 260) | 284 | 309 |

Expression of Homo sapiens C-reactive protein, pentraxin-related (CRP) HSCREACT transcripts which are detectable by amplicon as depicted in sequence name HSCREACT junc11-53F2R2 (SEQ ID NO:325) in different normal tissues:

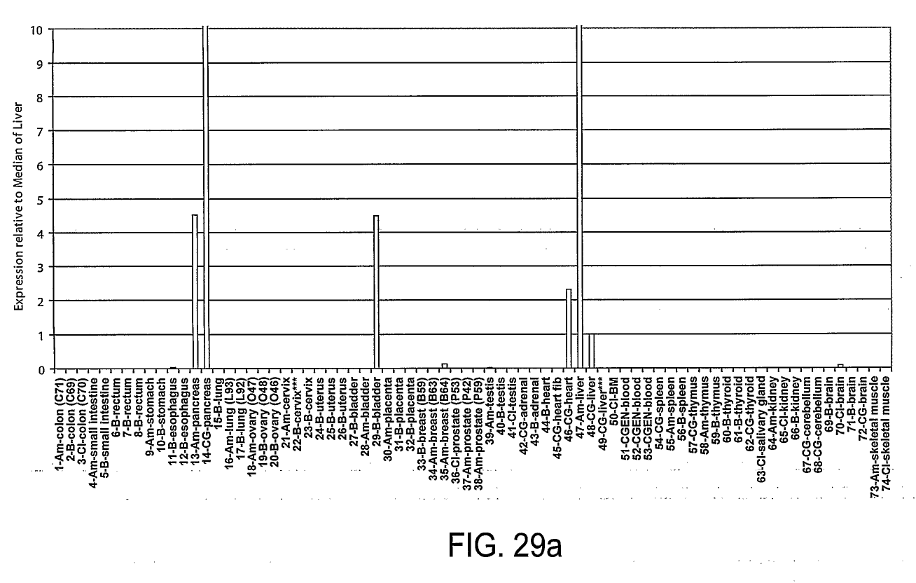

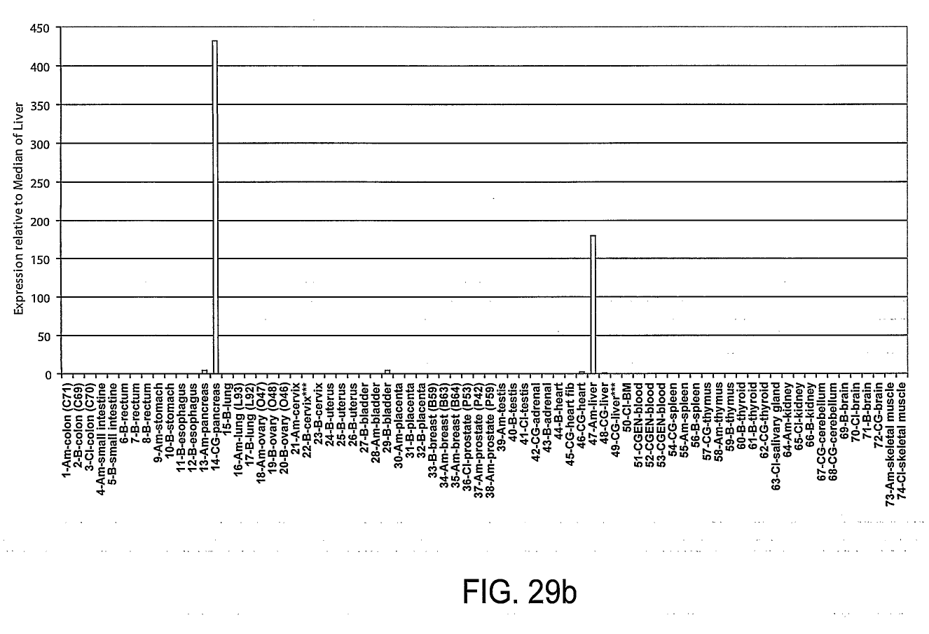

Expression of Homo sapiens C-reactive protein, pentraxin-related (CRP) transcripts detectable by or according to HSCREACT junc11-53F2R2 (SEQ ID NO:325) amplicon and primers HSCREACT junc11-53F2 (SEQ ID NO: 323) and HSCREACT junc11-53R2 (SEQ ID NO: 324) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the liver samples (Sample Nos. 47-49 Table 7, “Tissue samples in normal panel”), to obtain a value of relative expression of each sample relative to median of the liver samples. These data are plotted in FIGS. 29a and 29b in two different scales.

| | |
|---|---|
| HSCREACT junc11-53F2 Forward primer: | (SEQ ID NO: 323) |
| AACTGTCCTCGACCCTGCTTT | |
| HSCREACT junc11-53R2 Reverse primer: | (SEQ ID NO: 324) |
| GTGGCCTGGGTATATTGGGA | |
| Amplicon: | |

AACTGTCCTCGACCCTGCTTTCTTAATTTTATGGCTCTTCTGGGAAACTCCTCCCCTTTTCCACA
CGAACCTTGTGGGGCTGTGAATTCTTTCTTCATCCCCGCATTCCCAATATACCCAGGCCAC

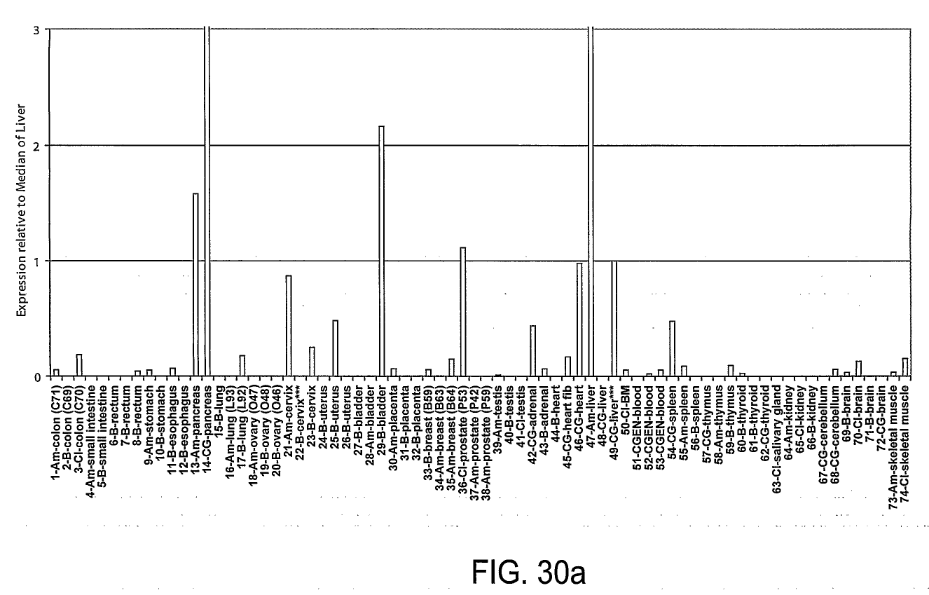

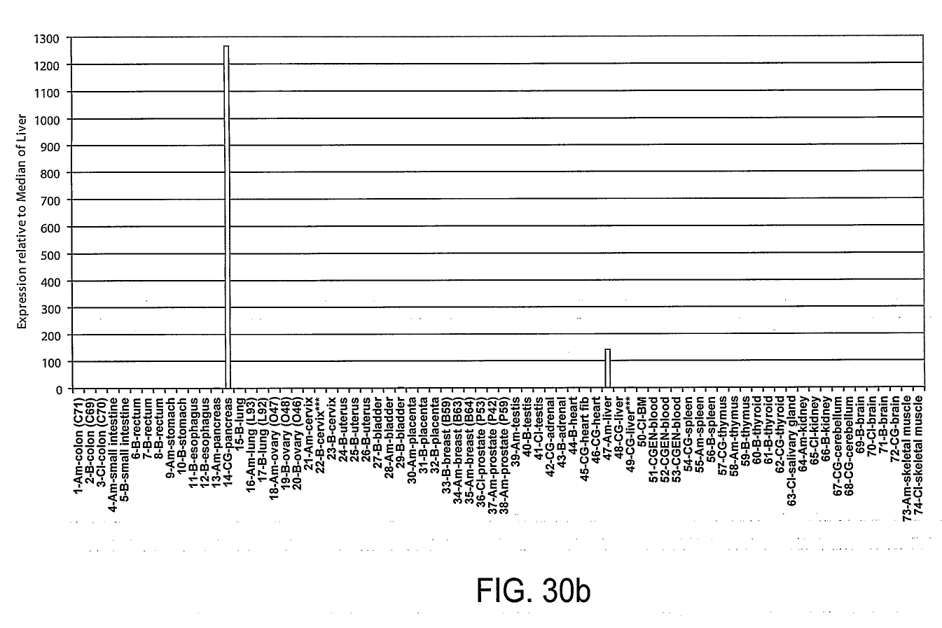

Expression of Homo sapiens C-reactive protein, pentraxin-related (CRP) HSCREACT transcripts which are detectable by amplicon as depicted in sequence name HSCREACT junc12-30F2R2 (SEQ ID NO:328) in different normal tissues
Expression of Homo sapiens C-reactive protein, pentraxin-related (CRP) transcripts detectable by or according to HSCREACT junc12-30F2R2 (SEQ ID NO:328) amplicon and primers HSCREACT junc12-30F2 (SEQ ID NO: 326) and HSCREACT junc12-30R2 (SEQ ID NO: 327) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the liver samples (Sample Nos. 47-49, Table 7, “Tissue samples in normal panel”), to obtain a value of relative expression of each sample relative to median of the liver samples. These data are plotted in FIGS. 30a and 30b below in two different scales.

| | |
|---|---|
| Forward primer HSCREACT junc12-30F2: | (SEQ ID NO: 326) |
| CTCGACCCGTGGATGAGATT | |
| Reverse primer HSCREACT junc12-30R2: | (SEQ ID NO: 327) |
| ACACTTCGCCTTGCACTTCA | |
| Amplicon: | |

CTCGACCCGTGGATGAGATTAACACCATCTATCTTGGCGGGCCCTTCAGTCCTAATGTCCTGA
ACTGGCGGGCACTGAAGTATGAAGTGCAAGGCGAAGTGT

Expression of Homo sapiens C-reactive protein, pentraxin-related (CRP) HSCREACT transcripts which are detectable by amplicon as depicted in sequence name HSCREACT junc12-53F2R2 (SEQ ID NO:331) in different normal tissues:

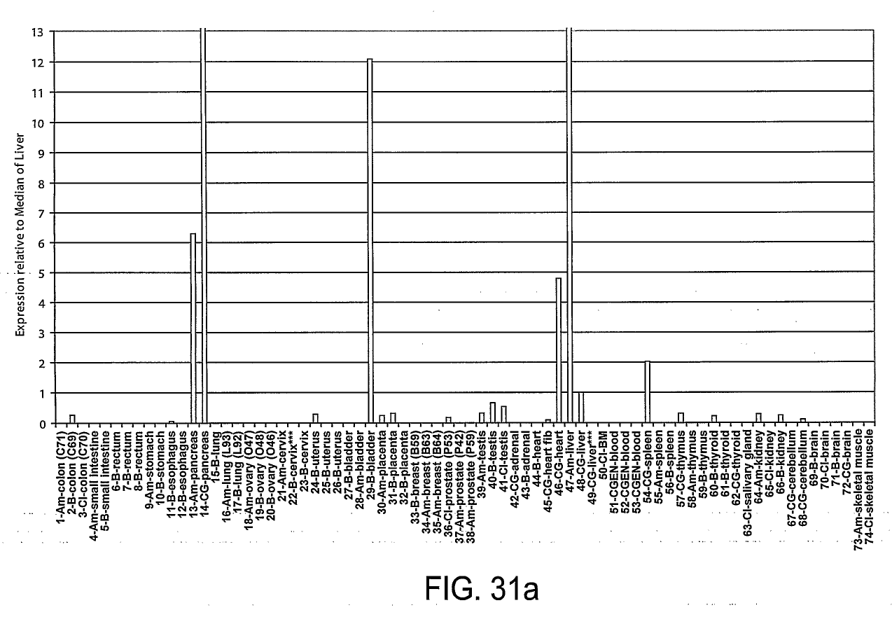

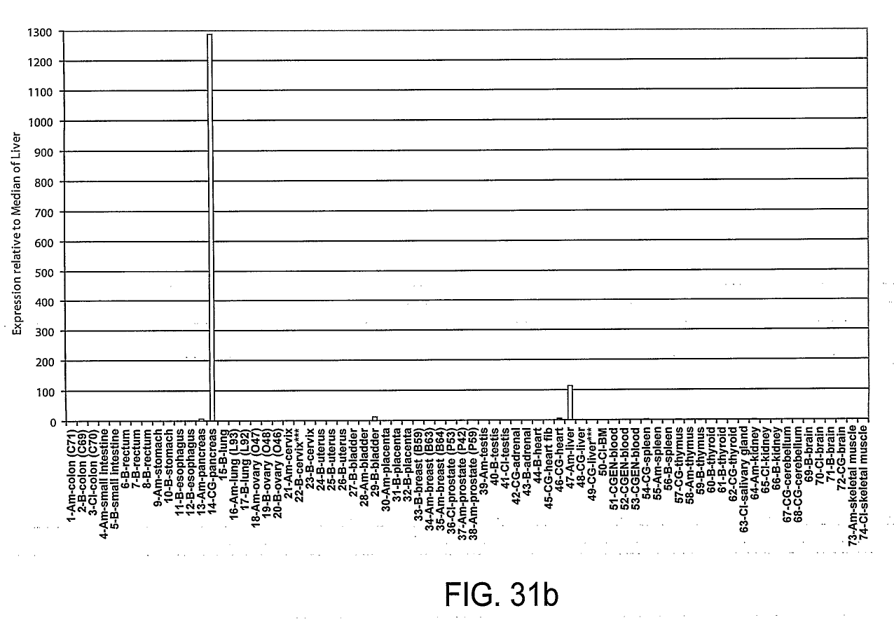

Expression of Homo sapiens C-reactive protein, pentraxin-related (CRP) transcripts detectable by or according to HSCREACT junc12-53F2R2 (SEQ ID NO:331) amplicon and primers HSCREACT junc12-53F2 (SEQ ID NO: 329) and HSCREACT junc12-53R2 (SEQ ID NO: 330) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the liver samples (Sample Nos. 47-49 Table 7, “Tissue samples in normal panel”), to obtain a value of relative expression of each sample relative to median of the liver samples. These data are plotted in FIGS. 31a and 31b below, in two different scales.

| | |
|---|---|
| Forward primer HSCREACT junc12-53F2: | (SEQ ID NO: 329) |
| CCTCGACCCGTGGTGCT | |
| Reverse primer HSCREACT junc12-53R2: | (SEQ ID NO: 330) |
| GTGGCCTGGGTATATTGGGA | |
| Amplicon: | |

CCTCGACCCGTGGTGCTTTCTTAATTTTATGGCTCTTCTGGGAAACTCCTCCCCTTTTCCACACG
AACCTTGTGGGGCTGTGAATTCTTTCTTCATCCCCGCATTCCCAATATACCCAGGCCAC

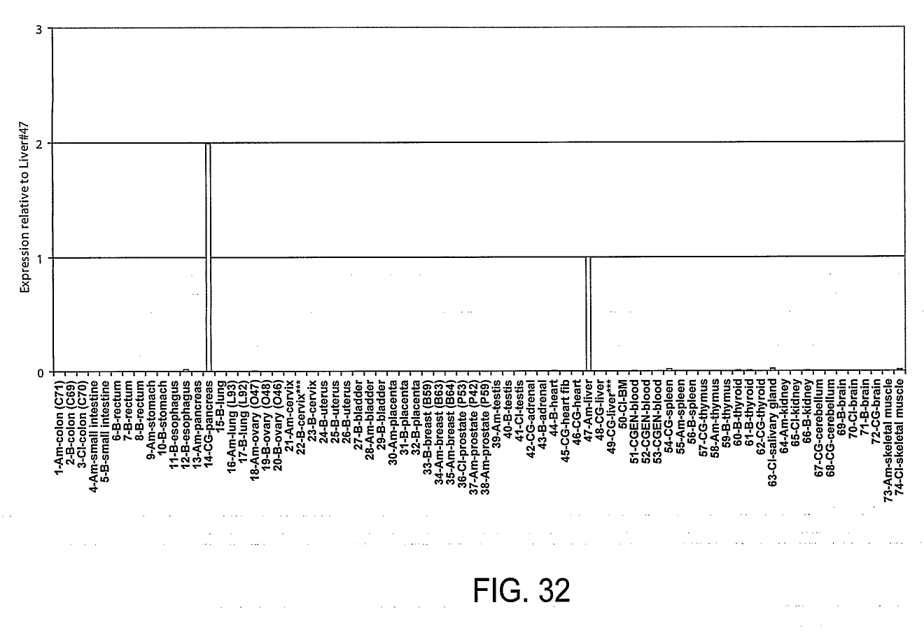

Expression of Homo sapiens C-reactive protein, pentraxin-related (CRP) HSCREACT transcripts which are detectable by amplicon as depicted in sequence name HSCREACT junc24-47F2R2 (SEQ ID NO:334) in different normal tissues:
Expression of Homo sapiens C-reactive protein, pentraxin-related (CRP) transcripts detectable by or according to HSCREACT junc24-47F2R2 (SEQ ID NO:334) amplicon and primers HSCREACT junc24-47F2 (SEQ ID NO: 332) and HSCREACT junc24-47R2 (SEQ ID NO: 333) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the quantity of the one liver sample that express this amplicon (Sample No. 47 Table 7, “Tissue samples in normal panel”), to obtain a value of relative expression of each sample relative to this liver sample. These data are plotted in FIG. 32.

| | |
|---|---|
| Forward primer HSCREACT junc24-47F2: | (SEQ ID NO: 332) |
| GCAGGATTCCTTCGTCTCAGA | |
| Reverse primer HSCREACT junc24-47R2: | (SEQ ID NO: 333) |
| GAGAAAGTGGAGGGACTGCG | |
| Amplicon: | |

GCAGGATTCCTTCGTCTCAGAATCAGGACACTGGCCAGGTGTCTGGTTTGGGTCCAGAGTGCT
CATCATCATGTCATAGAACTGCTGGGCCCAGGTCTCCTGAAATGGGAAGCCCAGCAATACCAC
GCAGTCCCTCCACTTTCTC

Expression of Homo sapiens C-reactive protein, pentraxin-related (CRP) HSCREACT transcripts which are detectable by amplicon as depicted in sequence name HSCREACT seg8-11 (SEQ ID NO: 337) in different normal tissues:

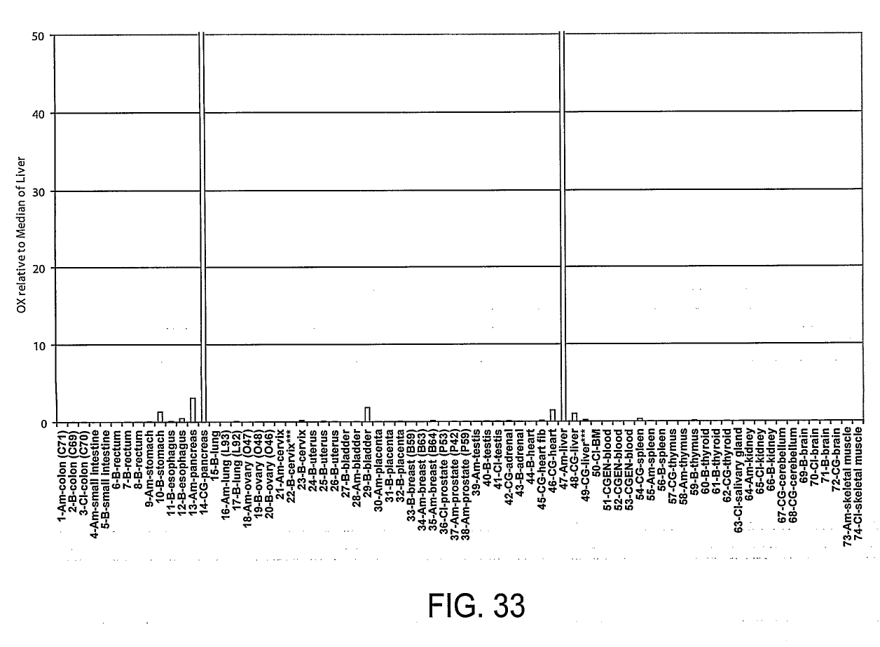

Expression of Homo sapiens C-reactive protein, pentraxin-related (CRP) transcripts detectable by or according to HSCREACT seg8-11 (SEQ ID NO: 337) amplicon and primers HSCREACT seg8-11F (SEQ ID NO: 335) and HSCREACT seg8-11R (SEQ ID NO: 336) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the liver samples (Sample Nos. 47-49 Table 7, “Tissue samples in normal panel”), to obtain a value of relative expression of each sample relative to median of the liver samples. These data are plotted in FIG. 33.

| | |
|---|---|
| Forward primer HSCREACT seg8-11F: | (SEQ ID NO: 335) |
| GAAGGCTTTTGTGTTTCCCAAA | |
| Reverse primer HSCREACT seg8-11R: | (SEQ ID NO: 336) |
| AGAAGTGGAGGCACACAGTGAA | |
| Amplicon: | (SEQ ID NO: 337) |

GAAGGCTTTTGTGTTTCCCAAAGAGTCGGATACTTCCTATGTATCCCTCAAAGCACCGTTAACG
AAGCCTCTCAAAGCCTTCACTGTGTGCCTCCACTTCT

### Description for Cluster HSSTROL3

Cluster HSSTROL3 features 8 transcript(s) and 17 segment(s) of interest, the names for which are given in Tables 120 and 121. The selected protein variants are given in table 122.

TABLE 120
Transcripts of interest
Transcript Name

HSSTROL3_PEA_1_T2 (SEQ ID NO: 338)
HSSTROL3_PEA_1_T5 (SEQ ID NO: 339)
HSSTROL3_PEA_1_T6 (SEQ ID NO: 340)
HSSTROL3_PEA_1_T8 (SEQ ID NO: 341)
HSSTROL3_PEA_1_T9 (SEQ ID NO: 342)
HSSTROL3_PEA_1_T10 (SEQ ID NO: 343)
HSSTROL3_PEA_1_T11 (SEQ ID NO: 344)
HSSTROL3_PEA_1_T12 (SEQ ID NO: 345)

TABLE 121
Segments of interest

| Segment Name | |
|---|---|
| HSSTROL3_PEA_1_node_6 (SEQ ID NO: 346) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| | and HSSTROL3_PEA_1_T12 (SEQ ID |
| | NO: 345) |
| HSSTROL3_PEA_1_node_10 (SEQ ID NO: 347) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| | and HSSTROL3_PEA_1_T12 (SEQ ID |
| | NO: 345) |
| HSSTROL3_PEA_1_node_12 (SEQ ID NO: 348) | HSSTROL3_PEA_1_T2 (SEQ ID NO: 338) |
| HSSTROL3_PEA_1_node_13 (SEQ ID NO: 349) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| | and HSSTROL3_PEA_1_T12 (SEQ ID |
| | NO: 345) |
| HSSTROL3_PEA_1_node_15 (SEQ ID NO: 350) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| | and HSSTROL3_PEA_1_T12 (SEQ ID |
| | NO: 345) |
| HSSTROL3_PEA_1_node_19 (SEQ ID NO: 351) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| | and HSSTROL3_PEA_1_T12 (SEQ ID |
| | NO: 345) |
| HSSTROL3_PEA_1_node_21 (SEQ ID NO: 352) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| | and HSSTROL3_PEA_1_T12 (SEQ ID |
| | NO: 345) |
| HSSTROL3_PEA_1_node_24 (SEQ ID NO: 353) | HSSTROL3_PEA_1_T6 (SEQ ID NO: 340), |
| | HSSTROL3_PEA_1_T8 (SEQ ID NO: 341) |
| | and HSSTROL3_PEA_1_T9 (SEQ ID |
| | NO: 342) |
| HSSTROL3_PEA_1_node_25 (SEQ ID NO: 354) | HSSTROL3_PEA_1_T6 (SEQ ID NO: 340) |
| | and HSSTROL3_PEA_1_T8 (SEQ ID |
| | NO: 341) |
| HSSTROL3_PEA_1_node_26 (SEQ ID NO: 355) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342) |
| | and HSSTROL3_PEA_1_T11 (SEQ ID |
| | NO: 344) |
| HSSTROL3_PEA_1_node_28 (SEQ ID NO: 356) | HSSTROL3_PEA_1_T5 (SEQ ID NO: 339), |
| | HSSTROL3_PEA_1_T6 (SEQ ID NO: 340), |
| | HSSTROL3_PEA_1_T9 (SEQ ID NO: 342) |
| | and HSSTROL3_PEA_1_T10 (SEQ ID |
| | NO: 343) |
| HSSTROL3_PEA_1_node_29 (SEQ ID NO: 357) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| | and HSSTROL3_PEA_1_T12 (SEQ ID |
| | NO: 345) |
| HSSTROL3_PEA_1_node_11 (SEQ ID NO: 358) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343) |
| | and HSSTROL3_PEA_1_T11 (SEQ ID |
| | NO: 344) |
| HSSTROL3_PEA_1_node_17 (SEQ ID NO: 359) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| | and HSSTROL3_PEA_1_T12 (SEQ ID |
| | NO: 345) |
| HSSTROL3_PEA_1_node_18 (SEQ ID NO: 360) | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| HSSTROL3_PEA_1_node_20 (SEQ ID NO: 361) | HSSTROL3_PEA_1_T11 (SEQ ID NO: 344) |
| HSSTROL3_PEA_1_node_27 (SEQ ID NO: 362) | HSSTROL3_PEA_1_T2 (SEQ ID NO: 338), |
| | HSSTROL3_PEA_1_T5 (SEQ ID NO: 339), |
| | HSSTROL3_PEA_1_T6 (SEQ ID NO: 340), |
| | HSSTROL3_PEA_1_T8 (SEQ ID NO: 341), |
| | HSSTROL3_PEA_1_T9 (SEQ ID NO: 342), |
| | HSSTROL3_PEA_1_T10 (SEQ ID NO: 343), |
| | HSSTROL3_PEA_1_T11 (SEQ ID NO: 344) |
| | and HSSTROL3_PEA_1_T12 (SEQ ID |
| | NO: 345) |

TABLE 122
Proteins of interest

| Protein Name | Corresponding Transcript(s) |
|---|---|
| HSSTROL3_PEA_1_P4 | HSSTROL3_PEA_1_T5 |
| (SEQ ID NO: 364) | (SEQ ID NO: 339) |
| HSSTROL3_PEA_1_P5 | HSSTROL3_PEA_1_T6 |
| (SEQ ID NO: 365) | (SEQ ID NO: 340); |
| | HSSTROL3_PEA_1_T8 |
| | (SEQ ID NO: 341); |
| | HSSTROL3_PEA_1_T9 |
| | (SEQ ID NO: 342) |
| HSSTROL3_PEA_1_P7 | HSSTROL3_PEA_1_T10 |
| (SEQ ID NO: 366) | (SEQ ID NO: 343) |
| HSSTROL3_PEA_1_P8 | HSSTROL3_PEA_1_T11 |
| (SEQ ID NO: 367) | (SEQ ID NO: 344) |
| HSSTROL3_PEA_1_P9 | HSSTROL3_PEA_1_T12 |
| (SEQ ID NO: 368) | (SEQ ID NO: 345) |
| HSSTROL3_PEA_1_P11 | HSSTROL3_PEA_1_T2 |
| (SEQ ID NO: 369) | (SEQ ID NO: 338) |

These sequences are variants of the known protein Stromelysin-3 precursor (SEQ ID NO:363) (SwissProt accession identifier MMP11_HUMAN (SEQ ID NO:363); known also according to the synonyms EC 3.4.24.-; Matrix metalloproteinase-11; MMP-11; ST3; SL-3), referred to herein as the previously known protein.

Protein Stromelysin-3 precursor (SEQ ID NO:363) is known or believed to have the following function(s): May play an important role in the progression of epithelial malignancies.

The following GO Annotation(s) apply to the previously known protein. The following annotation(s) were found: proteolysis and peptidolysis; developmental processes; morphogenesis, which are annotation(s) related to Biological Process; stromelysin 3; calcium binding; zinc binding; hydrolase, which are annotation(s) related to Molecular Function; and extracellular matrix, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of HSSTROL3) may optionally have one or more of the following utilities, as described with regard to the Table 123 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 123
Utilities for Variants of HSSTROL3

| Diagnostic entity | Rationale | Reference |
|---|---|---|
| Identification of | Successful identification of two year old menstrual | Ferri et al., |
| menstrual blood | bloodstain by using MMP-11 shorter amplicons (forensic | Forensic Sci. |
| stains, for | evaluation of cases with sexual offense) | 2004 |
| example for | | Nov; 49(6): 1387. |
| evaluation of | | |
| forensic evidence | | |
| Marker for | Stromelysin-3 was expressed (immunostained) within the | Perret et al., |
| aggressive | stromal and neoplastic cells of only 1 benign meningioma | Cancer. 2002 |
| meningioma | and 13 atypical meningiomas. The MIB-1 proliferation | Feb |
| | index was significantly higher in the meningiomas | 1; 94(3): 765-72 |
| | expressing ST3 (Student t test: P < 0.001). The invasion of | |
| | bone, muscle, and brain by meningiomas as well the | |
| | recurrence were statistically correlated with their ST3 | |
| | expression (Kruskal-Wallis nonparametric correlation test, P = | |
| | 0.001 and P = 0.008, respectively). | |
| Marker for | MMP-11 strongly immunostained neoplastic gemistocytic | Thorns et al., |
| astrocytoma | astrocytes while oligodendrocytic tumor regions showed | Anticancer Res. |
| (aggressive brain | only a low immunoreaction. | 2003 Sep- |
| tumor) | | Oct; 23(5A): 3937-44 |
| Marker for breast | The MMP-11 gene was originally identified by screening a | Duffy et al., Int |
| cancer | breast cancer cDNA library for genes that were expressed at | J Oncol |
| | higher levels in invasive carcinomas than in breast | 1998; 12: 1343-1348 |
| | fibroadenomas. It rises in stromal (fibroblast) cells adjacent | |
| | to breast carcinoma cells. High levels of stromelysin-3 have | |
| | been found to correlate with poor outcome in patients with | |
| | breast cancer. | |
| Marker for colon | Expressed in invasive colon carcinoma. ST3 expression | Rio et al., J |
| or rectum - | determined by immunohistochemistry was individually | Mammary |
| (colorectal) | related to females, distally located tumors, infiltrative | Gland Biol |
| carcinoma | growth pattern and microsatellite stability. No relationship | Neoplasia. |
| | was found with age, Dukes' stage, differentiation and | 1996 |
| | survival. | Apr; 1(2): 231-40 |
| | | Skoglund et al. |
| | | Oncology. |
| | | 2004; 67(1): 67-72 |
| Marker for | Genes up-regulated (cDNA microarrays) in endometrioid | Moreno-Bueno |
| endometrioid | carcinoma included genes known to be hormonally | et al., Cancer |
| carcinoma | regulated during the menstrual cycle and to be important in | Res. 2003 Sep |
| | endometrial homeostasis, such as MGB2, LTF, END1, and | 15; 63(18): 5697-702 |
| | MMP11. | |
| Marker for | Kaplan-Meier survival analysis showed that patients with | Sharma et al., |
| esophageal | ST-3-positive and TIMP-2-negative esophageal carcinoma | Oncology. |
| carcinoma | (immunohistochemical analysis) had a significantly shorter | 2004; 67(3-4): |
| | disease-free survival (median disease-free survival time of 4 | 300-9 |
| | months) as compared to patients in the other groups (median | |
| | disease-free survival time of 20 months; p = 0.0016) | |
| Marker for gastric | MMP11 is highly expressed in primary tumor of gastric | Deng et al., |
| cancer | cancer (RNA) | Biochem |
| | | Biophys Res |
| | | Commun. 2005 |
| | | Jan |
| | | 14; 326(2): 274-81 |
| Marker for head | Expressed in invasive head and neck carcinoma; | Rio et al., J |
| and neck | Matrix metalloproteinase 11, v-Ral, and integrin beta(4) | Mammary |
| carcinoma | were highly expressed in tumor cells of EBV-associated | Gland Biol |
| | nasopharyngeal carcinomas (mRNA microarray); | Neoplasia. |
| | Significantly higher MMP-11 levels were found in | 1996 |
| | squamous cell carcinoma tumors vs specimens of matched | Apr; 1(2): 231-40; |
| | normal mucosa. | Sriuranpong et |
| | | al., Clin Cancer |
| | | Res. 2004 Aug |
| | | 1; 10(15): 4944-58; |
| | | O- |
| | | Charoenrat et |
| | | al., Arch |
| | | Otolaryngol |
| | | Head Neck |
| | | Surg. 2001 |
| | | Jul; 127(7): 813-20 |
| Marker for non- | Overexpression (Northern blot) level of stromelysin 3 is | Delebecq et al., |
| small cell lung | related to the lymph node involvement in non-small cell | Clin Cancer |
| carcinoma | lung cancer. | Res. 2000 |
| | | Mar; 6(3): 1086-92 |
| Marker for | MMP-11 was up-regulated in osteoarthritis chondrocytes | Aigner et al., |
| osteoarthritis | and, interestingly, also in the early-stage samples. | Arthritis |
| | | Rheum. 2001 |
| | | Dec; 44(12): 2777-89 |
| Marker for | A significantly higher percentage of ovarian carcinomas | Mueller et al., |
| ovarian | than low malignant potential tumours expressed | Virchows Arch. |
| carcinoma | (immunohistochemistry, in situ hybridization) ST-3 in the | 2000 |
| | stroma adjacent to the tumour, with a correlation to | Dec; 437(6): 618-24 |
| | increasing tumour stage. | |
| Marker for | MMP-11 was immunohistochemicaly elevated in pancreatic | Jones et al., |
| pancreatic cancer | carcinoma. Real-time RT-PCR analysis showed that | Clin Cancer |
| | increased MMP-11 expression was strongly associated with | Res. 2004 Apr |
| | lymph node involvement (P = 0.0073). | 15; 10(8): 2832-45 |
| Marker for | Known protein was expressed only in thyroid tumor tissue | Wasenius et al., |
| papillary thyroid | when compared to normal thyroid tissue. Positive | Clin Cancer |
| carcinoma | immunohistochemistry in 87% of tumor samples. | Res. 2003 |
| | | Jan; 9(1): 68-75 |
| Marker for renal | Expression of the MMPs was significantly stronger (RT- | Hagemann et |
| cell carcinoma | PCR) in the carcinomas than in non-malignant tissues. High | al., J Cancer. |
| | levels were demonstrated particularly in clear cell RCC. | 2001 |
| | Expression in papillary RCCs was significantly lower. | Oct; 37(15): 1839-46 |
| Marker for | Apoptosis-related genes CASP10 and MMP11 were | Leivo et al., |
| salivary duct | overexpressed in salivary duct carcinoma, in accordance | Cancer Genet |
| carcinoma | with the typical tumor necrosis seen in this entity. The array | Cytogenet. |
| | results were validated by RT-PCR and | 2005 Jan |
| | immunohistochemistry. | 5; 156(2): 104-13 |
| Marker for | Immunohistochemical analysis of IGF-II and ST-3 protein | Singer et al., . J |
| sarcoma | expression in sarcomas confirmed abundant expression. | Clin |
| | | Endocrinol |
| | | Metab. 1997 |
| | | Jun; 82(6): 1917-22 |
| Marker for skin | Immunoreactivity was positive for ST-3 in fibroblastic cells | Thewes et al., |
| cancers: basal cell | surrounding morphealike basal cell carcinoma cells in 34 | Thewes |
| carcinoma | (68%) of 50 cases, whereas the epithelial tumor cells | |
| | themselves were negative. In none of the 12 cases of | |
| | desmoplastic trichoepithelioma was expression of ST-3 in | |
| | fibroblasts observed. | |
| Marker for skin | ST3 immunostained cells were observed in all cases of | Cribier et al., J |
| cancers: | dermatofibromas (100%), including the 10 giant cases, but | Am Acad |
| differential | never in dermatofibrosarcoma protuberans (0%). | Dermatol. 2002 |
| diagnosis of skin | | Mar; 46(3): 408-13 |
| fibrous tumors | | |
| Marker for skin | Stromelysin-3 was immunohistochemicaly expressed only | Asch et al., Am J |
| cancers: | by fibroblasts surrounding the tumors and not by epithelial | Dermatopathol. |
| squamous cell | cells. The proportion of tumors positively stained was 22% | 1999 |
| carcinoma (SCC) | of keratoacanthoma, 47% of randomly selected SCC, and | Apr; 21(2): 146-50 |
| and | 70% of metastatic SCC | |
| keratoacanthoma | | |
| Marker for | Carcinoma-in-situ (pTis) and noninvasive papillary tumor | Mueller et al., |
| urinary bladder | (pTa) of the urinary bladder staining for ST-3 was evaluated. | Virchows Arch. |
| cancer | None of 27 pTis lesions, 4 of 27 pTa tumors, and 41 of 56 | 2000 |
| | (73.2%) invasive carcinomas were positive for ST-3. | Dec; 437(6): 618-24 |
| | Statistically significant associations were found in the | |
| | invasive carcinomas between ST-3 expression and | |
| | lymphatic vessel invasion, an infiltrative invasive pattern, | |
| | and invasion into at least the muscle layer (pT2, 3, 4 v pT1). | |
| | The expression of ST-3 in frankly invasive tumors was | |
| | associated with a more aggressive tumor phenotype. | |
| Marker for | Prominent among the 67 genes overexpressed (by | Tsibris et al., |
| uterine | microarray analysis) in leiomyoma relative to control | Fertil Steril. |
| leiomyoma | myometrium were ... stromelysin-3, and TGFbeta3. | 2002 |
| | | Jul; 78(1): 114-21 |
| Marker for | Human atherosclerotic plaques (n = 7) express stromelysin-3 | Schonbeck et |
| vascular injury | in situ, whereas fatty streaks (n = 5) and normal arterial | al., Exp Med. |
| and/or | specimens (n = 5) contain little or no stromelysin-3. | 1999 Mar |
| atherosclerosis | Stromelysin-3 mRNA and protein colocalized with | 1; 189(5): 843-53 |
| | endothelial cells, smooth muscle cells, and macrophages | |
| | within the lesion. | |

According to other optional embodiments of the present invention, variants of this cluster according to the present invention (amino acid and/or nucleic acid sequences of HSSTROL3) may optionally have one or more of the following utilities, some of which are related to utilities described above. It should be noted that these utilities are optionally and preferably suitable for human and non-human animals as subjects, except where otherwise noted.

A non-limiting example of such a utility is diagnosing leiomyoma, especially of the uterus. Use of the known protein for such a utility is described in U.S. Pat. No. 6,583,167, hereby incorporated by reference as if fully set forth herein.

Another non-limiting example of such a utility is the diagnosis of hyperproliferative disorders such as cancer for example. Use of the known protein for such a utility is described in US Patent Application No. US20040110152, hereby incorporated by reference as if fully set forth herein.

HSSTROL3 according to the present invention may optionally undergo intergenic splicing with SMARCB1, to create a fusion protein. SMARCB1 encodes a protein which is part of a complex that relieves repressive chromatin structures, allowing the transcriptional machinery to access its targets more effectively. The encoded nuclear protein may also bind to and enhance the DNA joining activity of HIV-1 integrase. This gene has been found to be a tumor suppressor, and mutations in it have been associated with malignant rhabdoid tumors. Deletions of this gene are linked to chronic myeloid leukemia (CML) and multiple rhabdoid tumors, and are conserved in human solid carcinomas.

Cluster HSSTROL3_PEA_{—}1 belongs to a family of proteins which are known to have at least a certain number of related functions, including but not limited to, MMP24, MMP25, MMP14, MMP16, MMP3, MMP1, MMP10 and all matrix metalloproteinases. These functions are described below; one or more variants of cluster HSSTROL3_PEA_{—}1 may optionally have one or more diagnostic utilities related to these functions.

Midtrimester amniotic fluid matrix metalloproteinase-8 (MMP-8) levels above the 90th percentile are a marker for subsequent preterm premature rupture of membranes (Am J Obstet Gynecol. 2005 January; 192(1):109-13). Matrix metalloproteinase 1 gene polymorphism is a prognostic predictor of invasive cervical cancer (Gynecol Oncol. 2005 February; 96(2):314-9). Expression of matrix metalloproteinase activity in idiopathic dilated cardiomyopathy showed it to be a marker of cardiac dilatation (Mol Cell Biochem. 2004 September; 264(1-2):183-91). An autoantibody against matrix metalloproteinase-3 was found in patients with systemic sclerosis (Clin Exp Immunol. 2004 November; 138(2):357-63). Serum active matrix metalloproteinase-9 was found in patients with non-small cell lung cancer (Lung Cancer. 2004 November; 46(2):205-13). A combination assay with circulating vascular endothelial growth factor (VEGF)-C, matrix metalloproteinase-9, and VEGF was found to be useful for diagnosing lymph node metastasis in patients with non-small cell lung cancer (Ann Surg Oncol. 2004 October; 11(10):928-33. Epub 2004 Sep. 20).

There is an association between serum matrix metalloproteinase-3 concentration and the promoter 5A/6A polymorphism in patients with coronary heart disease (Zhongguo Wei Zhong Bing Ji Jiu Yi Xue. 2004 September; 16(9):536-9). MMP-9, TIMP-1, CD-34 and factor-8 were found to be prognostic markers for squamous cell carcinoma of the tongue (Oral Oncol. 2004 ep; 40(8):798-803). There is a relationship between plasma matrix metalloproteinase-9 and clinical cardiovascular risk factors, as well as echocardiographic left ventricular measures (Framingham Heart Study. Circulation. 2004 Jun. 15; 109(23):2850-6. Epub 2004 Jun. 1). There is clinicopathologic significance of plasma matrix metalloproteinase-2 and -9 levels in patients with undifferentiated nasopharyngeal carcinoma (Eur J Surg Oncol. 2004 June; 30(5):560-4). Matrix metalloproteinase-9 was found to be a potential marker for prognosis in head and neck squamous cell carcinoma (Clin Cancer Res. 2004 May 1; 10(9):3110-6).

Serum matrix metalloproteinase-3 may be useful for detection of systemic sclerosis (Arch Dermatol Res. 2004 June; 296(1):25-9. Epub 2004 Apr. 17). Matrix metalloproteinases-2 and -9 were found in serum, core needle biopsies and tissue specimens of prostate cancer patients (Virchows Arch. 2004 June; 444(6):518-26. Epub 2004 Apr. 16). Matrix metalloproteinases were found in patients with uterine smooth muscle tumors, after an immunohistochemical analysis of MMP-1 and MMP-2 protein expression in leiomyoma, uterine smooth muscle tumor of uncertain malignant potential, and leiomyosarcoma (J Soc Gynecol Investig. 2004 April; 11(3):182-6).; Matrix metalloproteinases 2 and 9 may be used in the determination of invasive potential of pancreatic tumors (Bull Exp Biol Med. 2003 November; 136(5):494-8). The expression of uteroglobin and matrix metalloproteinase-9 genes in endometrial cancer was found to have a relationship to estrogen and progesterone receptor status (Oncol Rep. 2004 February; 11(2):427-33).

Table 124 below describes diagnostic utilities for the cluster HSSTROL3 that were found through microarrays, including the statistical significance thereof and a reference. One or more HSSTROL3 variants according to the present invention may optionally have one or more of these utilities.

TABLE 124

| | Statistical | |
|---|---|---|
| Diagnostic utility | significance | reference |
| Gene over expressed in Lung cancers | 0.002 to 2.6E−9 | Beer DJ, Hanash SM Nature Medicine |
| (Squamous Cell Lung Carcinoma., Lung | | (2002) Gene-expression profiles predict |
| Adenocarcinoma, Carcinoid, Small Cell | | survival of patients with lung |
| Lung Cancer) (vs. normal lung) | | adenocarcinoma. |
| | | Bhattacharjee A, Meyerson M PNAS |
| | | (2001) Classification of human lung |
| | | carcinomas by mRNA expression profiling |
| | | reveals distinct. |
| | | Garber ME, Petersen I PNAS (2001) |
| | | Diversity of gene expression in |
| | | adenocarcinoma of the lung. |
| Gene over expressed in lung metastasis | 0.002 | Bhattacharjee A, Meyerson M PNAS |
| (vs. primary tumor). | | (2001) Classification of human lung |
| | | carcinomas by mRNA expression profiling |
| | | reveals distinct adenocarcinoma |
| | | subclasses. |
| Gene over expressed in hepatocellular | 6E−8 | Chen X, Brown PO Mol Biol Cell (2002) |
| carcinoma (vs. normal liver). | | Gene Expression Patterns in Human Liver |
| | | Cancers. |
| Gene over expressed in Adenoid Cystic | 4.1E−5 | Frierson HF Jr, Hampton GM Am J Pathol |
| Carcinoma of Salivary Gland (vs. normal | | (2002) Large Scale Molecular Analysis |
| salivary gland). | | Identifies Genes with Altered Expression |
| | | in Salivary Adenoid Cystic Carcinoma. |
| Gene over expressed in Pancreatic | 3.6E−4 to | Iacobuzio-Donahue CA, Goggins M Am J |
| Adenocarcinoma (vs. normal pancreases | 9.6E−4 | Pathol (2003) Exploration of global gene |
| and other cancers). | | expression patterns in pancreatic |
| | | adenocarcinoma using cDNA microarrays. |
| | | Ramaswamy S; Golub TR PNAS (2001) |
| | | Multiclass cancer diagnosis using tumor |
| | | gene expression signatures. |
| Gene over expressed in prostate metastasis | 0.016 | LaTulippe E, Gerald WL Cancer Research |
| (vs. primary prostate cancer). | | (2002) Comprehensive Gene Expression |
| | | Analysis of Prostate Cancer Reveals |
| | | Distinct Transcriptional Programs |
| | | Associated with Metastatic Disease |
| Gene over expressed in prostate cancer | 0.026 | Luo JH, Becich M Molecular |
| (vs. normal prostate). | | Carcinogenesis (2002) Gene expression |
| | | analysis of prostate cancers |
| Gene over expressed Colon | 5.3E−6 to | Notterman DA, Levine AJ Cancer Res |
| Adenocarcinoma (vs. normal colon and | 6.5E−6 | (2001) Transcriptional Gene Expression |
| other cancers) | | Profiles of Colorectal Adenoma, |
| | | Adenocarcinoma, and Normal Tissue |
| | | Examined by Oligonucleotide Arrays. |
| | | Ramaswamy S; Golub TR PNAS (2001) |
| | | Multiclass cancer diagnosis using tumor |
| | | gene expression signatures. |
| Gene over expressed in benign uterine | | GNF database |
| leiomyomas (vs, Normal myometrium, | | (http://www.ncbi.nlm.nih.gov/projects/geo/): |
| malignant uterine and extra-uterine | | GDS533, probe ID: X57766_at |
| leiomyosarcoma). | | |

Also, microarrays have shown that one or more HSSTROL3 variants according to the present invention may optionally be overexpressed in lung cancer, preferably non small cell lung cancer, preferably adenocarcinoma, squamous cell cancer or carcinoid.

Other non-limiting exemplary utilities for HSSTROL3 variants according to the present invention are described in greater detail below and also with regard to the previous section on clinical utility.

Cluster HSSTROL3 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

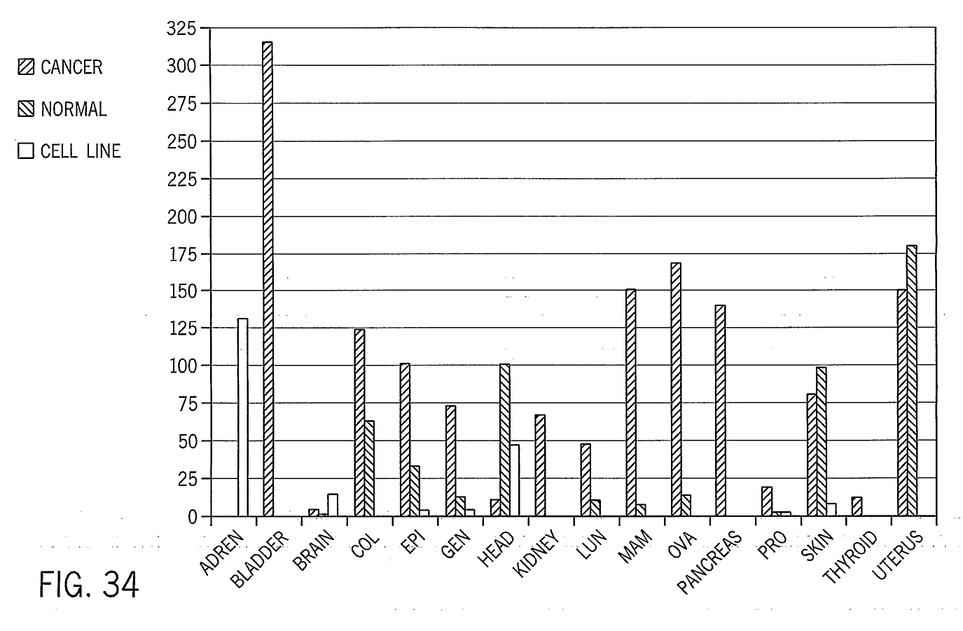

Overall, the following results were obtained as shown with regard to the histograms in FIG. 34 and Table 125. This cluster is overexpressed (at least at a minimum level) in the following pathological conditions: transitional cell carcinoma, epithelial malignant tumors, a mixture of malignant tumors from different tissues and pancreas carcinoma. P values and ratios for expression in cancerous tissue are described in table 126.

TABLE125
Normal tissue distribution

| Name of Tissue | Number |
|---|---|
| Adrenal | 0 |
| Bladder | 0 |
| Brain | 1 |
| Colon | 63 |
| Epithelial | 33 |
| General | 13 |
| head and neck | 101 |
| Kidney | 0 |
| Lung | 11 |
| Breast | 8 |
| Ovary | 14 |
| Pancreas | 0 |
| Prostate | 2 |
| Skin | 99 |
| Thyroid | 0 |
| Uterus | 181 |

TABLE 126
P values and ratios for expression in cancerous tissue

| Name of Tissue | P1 | P2 | SP1 | R3 | SP2 | R4 |
|---|---|---|---|---|---|---|
| adrenal | 1 | 4.6e−01 | 1 | 1.0 | 5.3e−01 | 1.9 |
| bladder | 2.7e−01 | 3.4e−01 | 3.3e−03 | 4.9 | 2.1e−02 | 3.3 |
| brain | 3.5e−01 | 2.6e−01 | 1 | 1.7 | 3.3e−01 | 2.8 |
| colon | 7.7e−02 | 1.5e−01 | 3.1e−01 | 1.4 | 5.2e−01 | 1.0 |
| epithelial | 1.2e−04 | 1.2e−02 | 1.3e−06 | 2.7 | 4.6e−02 | 1.4 |
| general | 5.4e−09 | 3.1e−05 | 1.8e−16 | 5.0 | 3.1e−07 | 2.6 |
| head and neck | 4.6e−01 | 4.3e−01 | 1 | 0.6 | 9.4e−01 | 0.7 |
| kidney | 2.5e−01 | 3.5e−01 | 1.1e−01 | 4.0 | 2.4e−01 | 2.8 |
| lung | 1.8e−01 | 4.5e−01 | 1.9e−01 | 2.7 | 5.1e−01 | 1.4 |
| breast | 2.0e−01 | 3.4e−01 | 7.3e−02 | 3.3 | 2.5e−01 | 2.0 |
| ovary | 2.6e−01 | 3.2e−01 | 2.2e−02 | 2.0 | 7.0e−02 | 1.6 |
| pancreas | 9.5e−02 | 1.8e−01 | 1.8e−04 | 7.8 | 1.6e−03 | 5.5 |
| prostate | 8.2e−01 | 7.8e−01 | 4.5e−01 | 1.8 | 5.6e−01 | 1.5 |
| skin | 5.2e−01 | 5.8e−01 | 7.1e−01 | 0.8 | 1 | 0.3 |
| Thyroid | 2.9e−01 | 2.9e−01 | 1 | 1.1 | 1 | 1.1 |
| uterus | 4.2e−01 | 8.0e−01 | 7.5e−01 | 0.6 | 9.9e−01 | 0.4 |

As noted above, cluster HSSTROL3 features 8 transcript(s), which were listed in Table 120 above. These transcript(s) encode for protein(s) which are variant(s) of protein Stromelysin-3 precursor (SEQ ID NO:363). A description of each variant protein according to the present invention is now provided.

Variant protein HSSTROL3_PEA_{—}1_P4 (SEQ ID NO:364) according to the present invention has an amino acid sequence; it is encoded by transcript(s) HSSTROL3_PEA_{—}1_T5 (SEQ ID NO:339). An alignment is given to the known protein (Stromelysin-3 precursor (SEQ ID NO:363)) at the end of the application. One or more alignments to one or more previously published protein sequences are given in the alignment table located on the attached CDROM. A brief description of the relationship of the variant protein according to the present invention to each such aligned protein is as follows:

Comparison report between HSSTROL3_PEA_{—}1_P4 (SEQ ID NO:364) and MMP11_HUMAN (SEQ ID NO:363):

1. An isolated chimeric polypeptide encoding for HSSTROL3_PEA_{—}1_P4 (SEQ ID NO:364), comprising a first amino acid sequence being at least 90% homologous to MAPAAWLRSAAARALLPPMLLLLLQPPPLLARALPPDVHHLHAERRGPQPWHAALPSSPAPAPAT QEAPRPASSLRPPRCGVPDPSDGLSARNRQKRFVLSGGRWEKTDLTYRILRFPWQLVQEQVRQTM AEALKVWSDVTPLTFTEVHEGRADIMIDFARYW corresponding to amino acids 1-163 of MMP11_HUMAN (SEQ ID NO:363), which also corresponds to amino acids 1-163 of HSSTROL3_PEA_{—}1_P4 (SEQ ID NO:364), a bridging amino acid H corresponding to amino acid 164 of HSSTROL3_PEA_{—}1_P4 (SEQ ID NO:364), a second amino acid sequence being at least 90% homologous to GDDLPFDGPGGILAHAFFPKTHREGDVHFDYDETWTIGDDQGTDLLQVAAHEFGHVLGLQHTTA AKALMSAFYTFRYPLSLSPDDCRGVQHLYGQPWPTVTSRTPALGPQAGIDTNEIAPLEPDAPPDAC EASFDAVSTIRGELFFFKAGFVWRLRGGQLQPGYPALASRHWQGLPSPVDAAFEDAQGHIWFFQG AQYWVYDGEKPVLGPAPLTELGLVRFPVHAALVWGPEKNKIYFFRGRDYWRFHPSTRRVDSPVP RRATDWRGVPSEIDAAFQDADG corresponding to amino acids 165-445 of MMP11_HUMAN (SEQ ID NO:363), which also corresponds to amino acids 165-445 of HSSTROL3_PEA_{—}1_P4 (SEQ ID NO:364), and a third amino acid sequence being at least 70%, optionally at least 80%, preferably at least 85%, more preferably at least 90% and most preferably at least 95% homologous to a polypeptide having the sequence ALGVRQLVGGGHSSRFSHLVVAGLPHACHRKSGSSSQVLCPEPSALLSVAG (SEQ ID NO: 652) corresponding to amino acids 446-496 of HSSTROL3_PEA_{—}1_P4 (SEQ ID NO:364), wherein said first amino acid sequence, bridging amino acid, second amino acid sequence and third amino acid sequence are contiguous and in a sequential order.

2. An isolated polypeptide encoding for a tail of HSSTROL3_PEA_{—}1_P4 (SEQ ID NO:364), comprising a polypeptide being at least 70%, optionally at least about 80%, preferably at least about 85%, more preferably at least about 90% and most preferably at least about 95% homologous to the sequence ALGVRQLVGGGHSSRFSHLVVAGLPHACHRKSGSSSQVLCPEPSALLSVAG (SEQ ID NO: 652) in HSSTROL3_PEA_{—}1_P4 (SEQ ID NO:364).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The variant protein has the following domains, as determined by using InterPro. The domains are described in Table 127:

TABLE 127
InterPro domain(s)

| InterPro ID | Domain description | Analysis type | Position(s) on protein |
|---|---|---|---|
| IPR001818 | Peptidase M10A and M12B, | FPrintScan | 130-145, 153-181, 212-237, |
| | matrixin and adamalysin | | 245-258, 75-88 |
| IPR000585 | Hemopexin repeat | HMMPfam | 298-341, 343-384, 387-434 |
| IPR001818 | Peptidase M10A and M12B, | HMMPfam | 98-204 |
| | matrixin and adamalysin | | |
| IPR000585 | Hemopexin repeat | HMMSmart | 298-341, 343-384, 387-434 |
| IPR006026 | Peptidase, metallopeptidases | HMMSmart | 101-259 |
| IPR000585 | Hemopexin repeat | ScanRegExp | 332-347 |
| IPR006025 | Peptidase M, neutral zinc | ScanRegExp | 212-221 |
| | metallopeptidases, zinc-binding | | |
| | site | | |
| IPR001818 | Peptidase M10A and M12B, | ScanRegExp | 78-85 |
| | matrixin and adamalysin | | |

Variant protein HSSTROL3_PEA_{—}1_P4 (SEQ ID NO:364) is encoded by the following transcript(s): HSSTROL3_PEA_{—}1_T5 (SEQ ID NO:339). The coding portion of transcript HSSTROL3_PEA_{—}1_T5 (SEQ ID NO:339) starts at position 24 and ends at position 1511. The transcript also has the following SNPs as listed in Table 128 (given according to their position on the nucleotide sequence, with the alternative nucleic acid listed; the last column indicates whether the SNP is known or not; the presence of known SNPs in variant protein HSSTROL3_PEA_{—}1_P4 (SEQ ID NO:364) sequence provides support for the deduced sequence of this variant protein according to the present invention).

TABLE 128
Nucleic acid SNPs

| SNP position on nucleotide | Alternative | |
|---|---|---|
| sequence | nucleic acid | Previously known SNP? |
| 136 | T -> C | Yes |
| 334 | G -> C | Yes |
| 663 | G -> | No |
| 699 | -> T | No |
| 992 | G -> C | Yes |
| 1528 | A -> G | Yes |
| 1710 | A -> G | Yes |
| 2251 | A -> G | Yes |
| 2392 | C -> | No |
| 2444 | C -> A | Yes |
| 2470 | A -> T | Yes |
| 2687 | -> G | No |
| 2696 | -> G | No |
| 2710 | C -> | No |
| 2729 | -> A | No |
| 2755 | T -> C | No |
| 2813 | A -> | No |
| 2813 | A -> C | No |
| 2963 | A -> | No |
| 2963 | A -> C | No |
| 2993 | T -> C | Yes |
| 3140 | -> T | No |

Variant protein HSSTROL3_PEA_{—}1_P5 (SEQ ID NO:365) according to the present invention has an amino acid sequence; it is encoded by transcript(s) HSSTROL3_PEA_{—}1_T6 (SEQ ID NO:340). An alignment is given to the known protein (Stromelysin-3 precursor (SEQ ID NO:363)) at the end of the application. One or more alignments to one or more previously published protein sequences are given in the alignment table located on the attached CDROM. A brief description of the relationship of the variant protein according to the present invention to each such aligned protein is as follows:

Comparison report between HSSTROL3_PEA_{—}1_P5 (SEQ ID NO:365) and MMP11_HUMAN (SEQ ID NO:363):

1. An isolated chimeric polypeptide encoding for HSSTROL3_PEA_{—}1_P5 (SEQ ID NO:365), comprising a first amino acid sequence being at least 90% homologous to MAPAAWLRSAAARALLPPMLLLLLQPPPLLARALPPDVHHLHAERRGPQPWHAALPSSPAPAPAT QEAPRPASSLRPPRCGVPDPSDGLSARNRQKRFVLSGGRWEKTDLTYRILRFPWQLVQEQVRQTM AEALKVWSDVTPLTFTEVHEGRADIMIDFARYW corresponding to amino acids 1-163 of MMP11_HUMAN (SEQ ID NO:363), which also corresponds to amino acids 1-163 of HSSTROL3_PEA_{—}1_P5 (SEQ ID NO:365), a bridging amino acid H corresponding to amino acid 164 of HSSTROL3_PEA_{—}1_P5 (SEQ ID NO:365), a second amino acid sequence being at least 90% homologous to GDDLPFDGPGGILAHAFFPKTHREGDVHFDYDETWTIGDDQGTDLLQVAAHEFGHVLGLQHTTA AKALMSAFYTFRYPLSLSPDDCRGVQHLYGQPWPTVTSRTPALGPQAGIDTNEIAPLEPDAPPDAC EASFDAVSTIRGELFFFKAGFVWRLRGGQLQPGYPALASRHWQGLPSPVDAAFEDAQGHIWFFQ corresponding to amino acids 165-358 of MMP11_HUMAN (SEQ ID NO:363), which also corresponds to amino acids 165-358 of HSSTROL3_PEA_{—}1_P5 (SEQ ID NO:365), and a third amino acid sequence being at least 70%, optionally at least 80%, preferably at least 85%, more preferably at least 90% and most preferably at least 95% homologous to a polypeptide having the sequence ELGFPSSTGRDESLEHCRCQGLHK (SEQ ID NO: 653) corresponding to amino acids 359-382 of HSSTROL3_PEA_{—}1_P5 (SEQ ID NO:365), wherein said first amino acid sequence, bridging amino acid, second amino acid sequence and third amino acid sequence are contiguous and in a sequential order.

2. An isolated polypeptide encoding for a tail of HSSTROL3_PEA_{—}1_P5 (SEQ ID NO:365), comprising a polypeptide being at least 70%, optionally at least about 80%, preferably at least about 85%, more preferably at least about 90% and most preferably at least about 95% homologous to the sequence ELGFPSSTGRDESLEHCRCQGLHK (SEQ ID NO: 653) in HSSTROL3_PEA_{—}1_P5 (SEQ ID NO:365).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The variant protein has the following domains, as determined by using InterPro. The domains are described in Table 129:

TABLE 129
InterPro domain(s)

| InterPro ID | Domain description | Analysis type | Position(s) on protein |
|---|---|---|---|
| IPR001818 | Peptidase M10A and M12B, | FPrintScan | 130-145, 153-181, 212-237, |
| | matrixin and adamalysin | | 245-258, 75-88 |
| IPR000585 | Hemopexin repeat | HMMPfam | 298-341 |
| IPR001818 | Peptidase M10A and M12B, | HMMPfam | 98-204 |
| | matrixin and adamalysin | | |
| IPR000585 | Hemopexin repeat | HMMSmart | 298-341 |
| IPR006026 | Peptidase, metallopeptidases | HMMSmart | 101-259 |
| IPR000585 | Hemopexin repeat | ScanRegExp | 332-347 |
| IPR006025 | Peptidase M, neutral zinc | ScanRegExp | 212-221 |
| | metallopeptidases, zinc-binding | | |
| | site | | |
| IPR001818 | Peptidase M10A and M12B, | ScanRegExp | 78-85 |
| | matrixin and adamalysin | | |
| IPR001818 | Peptidase M10A and M12B, | FPrintScan | 130-145, 153-181, 212-237, |
| | matrixin and adamalysin | | 245-258, 75-88 |
| IPR000585 | Hemopexin repeat | HMMPfam | 298-341 |
| IPR001818 | Peptidase M10A and M12B, | HMMPfam | 98-204 |
| | matrixin and adamalysin | | |
| IPR000585 | Hemopexin repeat | HMMSmart | 298-341 |
| IPR006026 | Peptidase, metallopeptidases | HMMSmart | 101-259 |
| IPR000585 | Hemopexin repeat | ScanRegExp | 332-347 |
| IPR006025 | Peptidase M, neutral zinc | ScanRegExp | 212-221 |
| | metallopeptidases, zinc-binding | | |
| | site | | |
| IPR001818 | Peptidase M10A and M12B, | ScanRegExp | 78-85 |
| | matrixin and adamalysin | | |
| IPR001818 | Peptidase M10A and M12B, | FPrintScan | 130-145, 153-181, 212-237, |
| | matrixin and adamalysin | | 245-258, 75-88 |
| IPR000585 | Hemopexin repeat | HMMPfam | 298-341 |
| IPR001818 | Peptidase M10A and M12B, | HMMPfam | 98-204 |
| | matrixin and adamalysin | | |
| IPR000585 | Hemopexin repeat | HMMSmart | 298-341 |
| IPR006026 | Peptidase, metallopeptidases | HMMSmart | 101-259 |
| IPR000585 | Hemopexin repeat | ScanRegExp | 332-347 |
| IPR006025 | Peptidase M, neutral zinc | ScanRegExp | 212-221 |
| | metallopeptidases, zinc-binding | | |
| | site | | |
| IPR001818 | Peptidase M10A and M12B, | ScanRegExp | 78-85 |
| | matrixin and adamalysin | | |

Variant protein HSSTROL3_PEA_{—}1_P5 (SEQ ID NO:365) is encoded by the following transcript(s): HSSTROL3_PEA_{—}1_T6 (SEQ ID NO:340). The coding portion of transcript HSSTROL3_PEA_{—}1_T6 (SEQ ID NO:340) starts at position 24 and ends at position 1169. The transcript also has the following SNPs as listed in Table 130 (given according to their position on the nucleotide sequence, with the alternative nucleic acid listed; the last column indicates whether the SNP is known or not; the presence of known SNPs in variant protein HSSTROL3_PEA_{—}1_P5 (SEQ ID NO:365) sequence provides support for the deduced sequence of this variant protein according to the present invention).

TABLE 130
Nucleic acid SNPs

| SNP position on | Alternative | Previously |
|---|---|---|
| nucleotide sequence | nucleic acid | known SNP? |
| 136 | T -> C | Yes |
| 334 | G -> C | Yes |
| 663 | G -> | No |
| 699 | -> T | No |
| 992 | G -> C | Yes |
| 1966 | A -> G | Yes |
| 2148 | A -> G | Yes |
| 2689 | A -> G | Yes |
| 2830 | C -> | No |
| 2882 | C -> A | Yes |
| 2908 | A -> T | Yes |
| 3125 | -> G | No |
| 3134 | -> G | No |
| 3148 | C -> | No |
| 3167 | -> A | No |
| 3193 | T -> C | No |
| 3251 | A -> | No |
| 3251 | A -> C | No |
| 3401 | A -> | No |
| 3401 | A -> C | No |
| 3431 | T -> C | Yes |
| 3578 | -> T | No |

Variant protein HSSTROL3_PEA_{—}1_P7 (SEQ ID NO:366) according to the present invention has an amino acid sequence; it is encoded by transcript(s) HSSTROL3_PEA_{—}1_T10 (SEQ ID NO:343). An alignment is given to the known protein (Stromelysin-3 precursor (SEQ ID NO:363)) at the end of the application. One or more alignments to one or more previously published protein sequences are given in the alignment table located on the attached CDROM. A brief description of the relationship of the variant protein according to the present invention to each such aligned protein is as follows:

Comparison report between HSSTROL3_PEA_{—}1_P7 (SEQ ID NO:366) and MMP11_HUMAN (SEQ ID NO:363):

1. An isolated chimeric polypeptide encoding for HSSTROL3_PEA_{—}1_P7 (SEQ ID NO:366), comprising a first amino acid sequence being at least 90% homologous to MAPAAWLRSAAARALLPPMLLLLLQPPPLLARALPPDVHHLHAERRGPQPWHAALPSSPAPAPAT QEAPRPASSLRPPRCGVPDPSDGLSARNRQKRFVLSGGRWEKTDLTYRILRFPWQLVQEQVRQTM AEALKVWSDVTPLTFTEVHEGRADIMIDFARYW corresponding to amino acids 1-163 of MMP11_HUMAN (SEQ ID NO:363), which also corresponds to amino acids 1-163 of HSSTROL3_PEA_{—}1_P7 (SEQ ID NO:366), a bridging amino acid H corresponding to amino acid 164 of HSSTROL3_PEA_{—}1_P7 (SEQ ID NO:366), a second amino acid sequence being at least 90% homologous to GDDLPFDGPGGILAHAFFPKTHREGDVHFDYDETWTIGDDQGTDLLQVAAHEFGHVLGLQHTTA AKALMSAFYTFRYPLSLSPDDCRGVQHLYGQPWPTVTSRTPALGPQAGIDTNEIAPLEPDAPPDAC EASFDAVSTIRGELFFFKAGFVWRLRGGQLQPGYPALASRHWQGLPSPVDAAFEDAQGHIWFFQG corresponding to amino acids 165-359 of MMP11_HUMAN (SEQ ID NO:363), which also corresponds to amino acids 165-359 of HSSTROL3_PEA_{—}1_P7 (SEQ ID NO:366), and a third amino acid sequence being at least 70%, optionally at least 80%, preferably at least 85%, more preferably at least 90% and most preferably at least 95% homologous to a polypeptide having the sequence TTGVSTPAPGV (SEQ ID NO: 654) corresponding to amino acids 360-370 of HSSTROL3_PEA_{—}1_P7 (SEQ ID NO:366), wherein said first amino acid sequence, bridging amino acid, second amino acid sequence and third amino acid sequence are contiguous and in a sequential order.

2. An isolated polypeptide encoding for a tail of HSSTROL3_PEA_{—}1_P7 (SEQ ID NO:366), comprising a polypeptide being at least 70%, optionally at least about 80%, preferably at least about 85%, more preferably at least about 90% and most preferably at least about 95% homologous to the sequence TTGVSTPAPGV (SEQ ID NO: 654) in HSSTROL3_PEA_{—}1_P7 (SEQ ID NO:366).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The variant protein has the following domains, as determined by using InterPro. The domains are described in Table 131:

TABLE 131
InterPro domain(s)

| InterPro ID | Domain description | Analysis type | Position(s) on protein |
|---|---|---|---|
| IPR001818 | Peptidase M10A and M12B, | FPrintScan | 130-145, 153-181, 212-237, |
| | matrixin and adamalysin | | 245-258, 75-88 |
| IPR000585 | Hemopexin repeat | HMMPfam | 298-341, 343-360 |
| IPR001818 | Peptidase M10A and M12B, | HMMPfam | 98-204 |
| | matrixin and adamalysin | | |
| IPR000585 | Hemopexin repeat | HMMSmart | 298-341 |
| IPR006026 | Peptidase, metallopeptidases | HMMSmart | 101-259 |
| IPR000585 | Hemopexin repeat | ScanRegExp | 332-347 |
| IPR006025 | Peptidase M, neutral zinc | ScanRegExp | 212-221 |
| | metallopeptidases, zinc-binding | | |
| | site | | |
| IPR001818 | Peptidase M10A and M12B, | ScanRegExp | 78-85 |
| | matrixin and adamalysin | | |

Variant protein HSSTROL3_PEA_{—}1_P7 (SEQ ID NO:366) is encoded by the following transcript(s): HSSTROL3_PEA_{—}1_T10 (SEQ ID NO:343). The coding portion of transcript HSSTROL3_PEA_{—}1_T10 (SEQ ID NO:343) starts at position 24 and ends at position 1133. The transcript also has the following SNPs as listed in Table 132 (given according to their position on the nucleotide sequence, with the alternative nucleic acid listed; the last column indicates whether the SNP is known or not; the presence of known SNPs in variant protein HSSTROL3_PEA_{—}1_P7 (SEQ ID NO:366) sequence provides support for the deduced sequence of this variant protein according to the present invention).

TABLE 132
Nucleic acid SNPs

| SNP position on | Alternative | Previously |
|---|---|---|
| nucleotide sequence | nucleic acid | known SNP? |
| 136 | T -> C | Yes |
| 334 | G -> C | Yes |
| 663 | G -> | No |
| 699 | -> T | No |
| 992 | G -> C | Yes |
| 1386 | A -> G | Yes |
| 1568 | A -> G | Yes |
| 2109 | A -> G | Yes |
| 2250 | C -> | No |
| 2302 | C -> A | Yes |
| 2328 | A -> T | Yes |
| 2545 | -> G | No |
| 2554 | -> G | No |
| 2568 | C -> | No |
| 2587 | -> A | No |
| 2613 | T -> C | No |
| 2671 | A -> | No |
| 2671 | A -> C | No |
| 2821 | A -> | No |
| 2821 | A -> C | No |
| 2851 | T -> C | Yes |
| 2998 | -> T | No |

Variant protein HSSTROL3_PEA_{—}1_P8 (SEQ ID NO:367) according to the present invention has an amino acid sequence; it is encoded by transcript(s) HSSTROL3_PEA_{—}1_T11 (SEQ ID NO:344). An alignment is given to the known protein (Stromelysin-3 precursor (SEQ ID NO:363)) at the end of the application. One or more alignments to one or more previously published protein sequences are given in the alignment table located on the attached CDROM. A brief description of the relationship of the variant protein according to the present invention to each such aligned protein is as follows:

Comparison report between HSSTROL3_PEA_{—}1_P8 (SEQ ID NO:367) and MMP11_HUMAN (SEQ ID NO:363):

1. An isolated chimeric polypeptide encoding for HSSTROL3_PEA_{—}1_P8 (SEQ ID NO:367), comprising a first amino acid sequence being at least 90% homologous to MAPAAWLRSAAARALLPPMLLLLLQPPPLLARALPPDVHHLHAERRGPQPWHAALPSSPAPAPAT QEAPRPASSLRPPRCGVPDPSDGLSARNRQKRFVLSGGRWEKTDLTYRILRFPWQLVQEQVRQTM AEALKVWSDVTPLTFTEVHEGRADIMIDFARYW corresponding to amino acids 1-163 of MMP11_HUMAN (SEQ ID NO:363), which also corresponds to amino acids 1-163 of HSSTROL3_PEA_{—}1_P8 (SEQ ID NO:367), a bridging amino acid H corresponding to amino acid 164 of HSSTROL3_PEA_{—}1_P8 (SEQ ID NO:367), a second amino acid sequence being at least 90% homologous to GDDLPFDGPGGILAHAFFPKTHREGDVHFDYDETWTIGDDQGTDLLQVAAHEFGHVLGLQHTTA AKALMSAFYTFRYPLSLSPDDCRGVQHLYGQPWPTVTSRTPALGPQAGIDTNEIAPLE corresponding to amino acids 165-286 of MMP11_HUMAN (SEQ ID NO:363), which also corresponds to amino acids 165-286 of HSSTROL3_PEA_{—}1_P8 (SEQ ID NO:367), and a third amino acid sequence being at least 70%, optionally at least 80%, preferably at least 85%, more preferably at least 90% and most preferably at least 95% homologous to a polypeptide having the sequence VRPCLPVPLLLCWPL (SEQ ID NO: 655) corresponding to amino acids 287-301 of HSSTROL3_PEA_{—}1_P8 (SEQ ID NO:367), wherein said first amino acid sequence, bridging amino acid, second amino acid sequence and third amino acid sequence are contiguous and in a sequential order.

2. An isolated polypeptide encoding for a tail of HSSTROL3_PEA_{—}1_P8 (SEQ ID NO:367), comprising a polypeptide being at least 70%, optionally at least about 80%, preferably at least about 85%, more preferably at least about 90% and most preferably at least about 95% homologous to the sequence VRPCLPVPLLLCWPL (SEQ ID NO: 655) in HSSTROL3_PEA_{—}1_P8 (SEQ ID NO:367).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The variant protein has the following domains, as determined by using InterPro. The domains are described in Table 133:

TABLE 133
InterPro domain(s)

| InterPro ID | Domain description | Analysis type | Position(s) on protein |
|---|---|---|---|
| IPR001818 | Peptidase M10A and M12B, | FPrintScan | 130-145, 153-181, 212-237, |
| | matrixin and adamalysin | | 245-258, 75-88 |
| IPR001818 | Peptidase M10A and M12B, | HMMPfam | 98-204 |
| | matrixin and adamalysin | | |
| IPR006026 | Peptidase, metallopeptidases | HMMSmart | 101-259 |
| IPR006025 | Peptidase M, neutral zinc | ScanRegExp | 212-221 |
| | metallopeptidases, zinc-binding | | |
| | site | | |
| IPR001818 | Peptidase M10A and M12B, | ScanRegExp | 78-85 |
| | matrixin and adamalysin | | |

Variant protein HSSTROL3_PEA_{—}1_P8 (SEQ ID NO:367) is encoded by the following transcript(s): HSSTROL3_PEA_{—}1_T11 (SEQ ID NO:344). The coding portion of transcript HSSTROL3_PEA_{—}1_T11 (SEQ ID NO:344) starts at position 24 and ends at position 926. The transcript also has the following SNPs as listed in Table 134 (given according to their position on the nucleotide sequence, with the alternative nucleic acid listed; the last column indicates whether the SNP is known or not; the presence of known SNPs in variant protein HSSTROL3_PEA_{—}1_P8 (SEQ ID NO:367) sequence provides support for the deduced sequence of this variant protein according to the present invention).

TABLE 134
Nucleic acid SNPs

| SNP position on | Alternative | Previously |
|---|---|---|
| nucleotide sequence | nucleic acid | known SNP? |
| 136 | T -> C | Yes |
| 334 | G -> C | Yes |
| 663 | G -> | No |
| 699 | -> T | No |
| 935 | G -> A | Yes |
| 948 | G -> A | Yes |
| 1084 | G -> C | Yes |
| 1557 | C -> | No |
| 1609 | C -> A | Yes |
| 1635 | A -> T | Yes |
| 1852 | -> G | No |
| 1861 | -> G | No |
| 1875 | C -> | No |
| 1894 | -> A | No |
| 1920 | T -> C | No |
| 1978 | A -> | No |
| 1978 | A -> C | No |
| 2128 | A -> | No |
| 2128 | A -> C | No |
| 2158 | T -> C | Yes |
| 2305 | -> T | No |

Variant protein HSSTROL3_PEA_{—}1_P9 (SEQ ID NO:368) according to the present invention has an amino acid sequence; it is encoded by transcript(s) HSSTROL3_PEA_{—}1_T12 (SEQ ID NO:345). An alignment is given to the known protein (Stromelysin-3 precursor (SEQ ID NO:363)) at the end of the application. One or more alignments to one or more previously published protein sequences are given in the alignment table located on the attached CDROM. A brief description of the relationship of the variant protein according to the present invention to each such aligned protein is as follows:

Comparison report between HSSTROL3_PEA_{—}1_P9 (SEQ ID NO:368) and MMP11_HUMAN (SEQ ID NO:363):

1. An isolated chimeric polypeptide encoding for HSSTROL3_PEA_{—}1_P9 (SEQ ID NO:368), comprising a first amino acid sequence being at least 90% homologous to MAPAAWLRSAAARALLPPMLLLLLQPPPLLARALPPDVHHLHAERRGPQPWHAALPSSPAPAPAT QEAPRPASSLRPPRCGVPDPSDGLSARNRQK corresponding to amino acids 1-96 of MMP11_HUMAN (SEQ ID NO:363), which also corresponds to amino acids 1-96 of HSSTROL3_PEA_{—}1_P9 (SEQ ID NO:368), a second amino acid sequence being at least 90% homologous to RILRFPWQLVQEQVRQTMAEALKVWSDVTPLTFTEVHEGRADIMIDFARYW corresponding to amino acids 113-163 of MMP11_HUMAN (SEQ ID NO:363), which also corresponds to amino acids 97-147 of HSSTROL3_PEA_{—}1_P9 (SEQ ID NO:368), a bridging amino acid H corresponding to amino acid 148 of HSSTROL3_PEA_{—}1_P9 (SEQ ID NO:368), a third amino acid sequence being at least 90% homologous to GDDLPFDGPGGILAHAFFPKTHREGDVHFDYDETWTIGDDQGTDLLQVAAHEFGHVLGLQHTTA AKALMSAFYTFRYPLSLSPDDCRGVQHLYGQPWPTVTSRTPALGPQAGIDTNEIAPLEPDAPPDAC EASFDAVSTIRGELFFFKAGFVWRLRGGQLQPGYPALASRHWQGLPSPVDAAFEDAQGHIWFFQG corresponding to amino acids 165-359 of MMP11_HUMAN (SEQ ID NO:363), which also corresponds to amino acids 149-343 of HSSTROL3_PEA_{—}1_P9 (SEQ ID NO:368), and a fourth amino acid sequence being at least 70%, optionally at least 80%, preferably at least 85%, more preferably at least 90% and most preferably at least 95% homologous to a polypeptide having the sequence TTGVSTPAPGV (SEQ ID NO: 654) corresponding to amino acids 344-354 of HSSTROL3_PEA_{—}1_P9 (SEQ ID NO:368), wherein said first amino acid sequence, second amino acid sequence, bridging amino acid, third amino acid sequence and fourth amino acid sequence are contiguous and in a sequential order.

2. An isolated chimeric polypeptide encoding for an edge portion of HSSTROL3_PEA_{—}1_P9 (SEQ ID NO:368), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KR, having a structure as follows: a sequence starting from any of amino acid numbers 96−x to 96; and ending at any of amino acid numbers 97+((n−2)−x), in which x varies from 0 to n−2.

3. An isolated polypeptide encoding for a tail of HSSTROL3_PEA_{—}1_P9 (SEQ ID NO:368), comprising a polypeptide being at least 70%, optionally at least about 80%, preferably at least about 85%, more preferably at least about 90% and most preferably at least about 95% homologous to the sequence TTGVSTPAPGV (SEQ ID NO: 654) in HSSTROL3_PEA_{—}1_P9 (SEQ ID NO:368).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The variant protein has the following domains, as determined by using InterPro. The domains are described in Table 135:

TABLE 135
InterPro domain(s)

| InterPro ID | Domain description | Analysis type | Position(s) on protein |
|---|---|---|---|
| IPR001818 | Peptidase M10A and M12B, | FPrintScan | 114-129, 137-165, 196-221, |
| | matrixin and adamalysin | | 229-242, 75-88 |
| IPR000585 | Hemopexin repeat | HMMPfam | 282-325, 327-344 |
| IPR001818 | Peptidase M10A and M12B, | HMMPfam | 104-188 |
| | matrixin and adamalysin | | |
| IPR000585 | Hemopexin repeat | HMMSmart | 282-325 |
| IPR006026 | Peptidase, metallopeptidases | HMMSmart | 87-243 |
| IPR000585 | Hemopexin repeat | ScanRegExp | 316-331 |
| IPR006025 | Peptidase M, neutral zinc | ScanRegExp | 196-205 |
| | metallopeptidases, zinc-binding | | |
| | site | | |
| IPR001818 | Peptidase M10A and M12B, | ScanRegExp | 78-85 |
| | matrixin and adamalysin | | |

Variant protein HSSTROL3_PEA_{—}1_P9 (SEQ ID NO:368) is encoded by the following transcript(s): HSSTROL3_PEA_{—}1_T12 (SEQ ID NO:345). The coding portion of transcript HSSTROL3_PEA_{—}1_T12 (SEQ ID NO:345) starts at position 24 and ends at position 1085. The transcript also has the following SNPs as listed in Table 136 (given according to their position on the nucleotide sequence, with the alternative nucleic acid listed; the last column indicates whether the SNP is known or not; the presence of known SNPs in variant protein HSSTROL3_PEA_{—}1_P9 (SEQ ID NO:368) sequence provides support for the deduced sequence of this variant protein according to the present invention).

TABLE 136
Nucleic acid SNPs

| SNP position on | Alternative | Previously |
|---|---|---|
| nucleotide sequence | nucleic acid | known SNP? |
| 136 | T -> C | Yes |
| 615 | G -> | No |
| 651 | -> T | No |
| 944 | G -> C | Yes |
| 1275 | C -> | No |
| 1327 | C -> A | Yes |
| 1353 | A -> T | Yes |
| 1570 | -> G | No |
| 1579 | -> G | No |
| 1593 | C -> | No |
| 1612 | -> A | No |
| 1638 | T -> C | No |
| 1696 | A -> | No |
| 1696 | A -> C | No |
| 1846 | A -> | No |
| 1846 | A -> C | No |
| 1876 | T -> C | Yes |
| 2023 | -> T | No |

Variant protein HSSTROL3_PEA_{—}1_P11 (SEQ ID NO:369) according to the present invention has an amino acid sequence; it is encoded by transcript(s) HSSTROL3_PEA_{—}1_T2 (SEQ ID NO:338). An alignment is given to the known protein (Stromelysin-3 precursor (SEQ ID NO:363)) at the end of the application. One or more alignments to one or more previously published protein sequences are given in the alignment table located on the attached CDROM. A brief description of the relationship of the variant protein according to the present invention to each such aligned protein is as follows:

Comparison report between HSSTROL3_PEA_{—}1_P11 (SEQ ID NO:369) and MMP11_HUMAN (SEQ ID NO:363):

1. An isolated chimeric polypeptide encoding for HSSTROL3_PEA_{—}1_P11 (SEQ ID NO:369), comprising a first amino acid sequence being at least 90% homologous to MAPAAWLRSAAARALLPPMLLLLLQPPPLLARALPPDVHHLHAERRGPQPWHAALPSSPAPAPAT QEAPRPASSLRPPRCGVPDPSDGLSARNRQKRFVLSGGRWEKTDLTYR corresponding to amino acids 1-113 of MMP11_HUMAN (SEQ ID NO:363), which also corresponds to amino acids 1-113 of HSSTROL3_PEA_{—}1_P11 (SEQ ID NO:369).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The variant protein has the following domains, as determined by using InterPro. The domains are described in Table 137:

TABLE 137
InterPro domain(s)

| | | | Position(s) |
|---|---|---|---|
| InterPro ID | Domain description | Analysis type | on protein |
| IPR001818 | Peptidase M10A and M12B, | ScanRegExp | 78-85 |
| | matrixin and adamalysin | | |

Variant protein HSSTROL3_PEA_{—}1_P11 (SEQ ID NO:369) is encoded by the following transcript(s): HSSTROL3_PEA_{—}1_T2 (SEQ ID NO:338). The coding portion of transcript HSSTROL3_PEA_{—}1_T2 (SEQ ID NO:338) starts at position 24 and ends at position 362. The transcript also has the following SNPs as listed in Table 138 (given according to their position on the nucleotide sequence, with the alternative nucleic acid listed; the last column indicates whether the SNP is known or not; the presence of known SNPs in variant protein HSSTROL3_PEA_{—}1_P11 (SEQ ID NO:369) sequence provides support for the deduced sequence of this variant protein according to the present invention).

TABLE 138
Nucleic acid SNPs

| SNP position on | Alternative | Previously |
|---|---|---|
| nucleotide sequence | nucleic acid | known SNP? |
| 136 | T -> C | Yes |
| 334 | G -> C | Yes |
| 603 | T -> A | No |
| 900 | G -> A | Yes |
| 1025 | G -> A | Yes |
| 1257 | G -> A | Yes |
| 1605 | G -> | No |
| 1641 | -> T | No |
| 1934 | G -> C | Yes |
| 2407 | C -> | No |
| 2459 | C -> A | Yes |
| 2485 | A -> T | Yes |
| 2702 | -> G | No |
| 2711 | -> G | No |
| 2725 | C -> | No |
| 2744 | -> A | No |
| 2770 | T -> C | No |
| 2828 | A -> | No |
| 2828 | A -> C | No |
| 2978 | A -> | No |
| 2978 | A -> C | No |
| 3008 | T -> C | Yes |
| 3155 | -> T | No |

Table 139 below describes the starting and ending position of HSSTROL3_PEA_{—}1_node_{—}20 (SEQ ID NO:361) on each of the relevant transcripts. Experimental results for this segment are described below.

TABLE 139
Segment location on transcripts

| | Segment | Segment |
|---|---|---|
| | starting | ending |
| Transcript name | position | position |
| HSSTROL3_PEA_1_T11 (SEQ ID NO: 344) | 882 | 973 |

Table 140 below describes the starting and ending position of HSSTROL3_PEA_{—}1_node_{—}21 (SEQ ID NO:352) on each of the relevant transcripts. Experimental results for this segment are described below.

TABLE 140
Segment location on transcripts

| | Segment | Segment |
|---|---|---|
| | starting | ending |
| Transcript name | position | position |
| HSSTROL3_PEA_1_T2 (SEQ ID NO: 338) | 1824 | 2040 |
| HSSTROL3_PEA_1_T5 (SEQ ID NO: 339) | 882 | 1098 |
| HSSTROL3_PEA_1_T6 (SEQ ID NO: 340) | 882 | 1098 |
| HSSTROL3_PEA_1_T8 (SEQ ID NO: 341) | 882 | 1098 |
| HSSTROL3_PEA_1_T9 (SEQ ID NO: 342) | 882 | 1098 |
| HSSTROL3_PEA_1_T10 (SEQ ID NO: 343) | 882 | 1098 |
| HSSTROL3_PEA_1_T11 (SEQ ID NO: 344) | 974 | 1190 |
| HSSTROL3_PEA_1_T12 (SEQ ID NO: 345) | 834 | 1050 |

Table 141 below describes the starting and ending position of HSSTROL3_PEA_{—}1_node_{—}24 (SEQ ID NO:353) on each of the relevant transcripts. Experimental results for this segment are described below.

TABLE 141
Segment location on transcripts

| | Segment | Segment |
|---|---|---|
| | starting | ending |
| Transcript name | position | position |
| HSSTROL3_PEA_1_T6 (SEQ ID NO: 340) | 1099 | 1236 |
| HSSTROL3_PEA_1_T8 (SEQ ID NO: 341) | 1099 | 1236 |
| HSSTROL3_PEA_1_T9 (SEQ ID NO: 342) | 1099 | 1236 |

Table 142 below describes the starting and ending position of HSSTROL3_PEA_{—}1_node_{—}25 (SEQ ID NO:354) on each of the relevant transcripts. Experimental results for this segment are described below.

TABLE 142
Segment location on transcripts

| | Segment | Segment |
|---|---|---|
| | starting | ending |
| Transcript name | position | position |
| HSSTROL3_PEA_1_T6 (SEQ ID NO: 340) | 1237 | 1536 |
| HSSTROL3_PEA_1_T8 (SEQ ID NO: 341) | 1237 | 1536 |

Expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in normal and cancerous breast tissues

Expression of Homo sapiens matrix metalloproteinase 11 (stromelysin 3) (MMP11) transcripts detectable by or according to junc21-26-HSSTROL3 junc21-26 (SEQ ID NO:370) amplicon and primers HSSTROL3 junc21-26F (SEQ ID NO:372) and HSSTROL3 junc21-26R (SEQ ID NO:371) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and G6PD (GenBank Accession No. NM_{—}000402 (SEQ ID NO:8); G6PD amplicon (SEQ ID NO: 44)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67, Table 6, above, “Tissue samples in breast cancer testing panel”), to obtain a value of fold up-regulation for each sample relative to median of the normal PM samples.

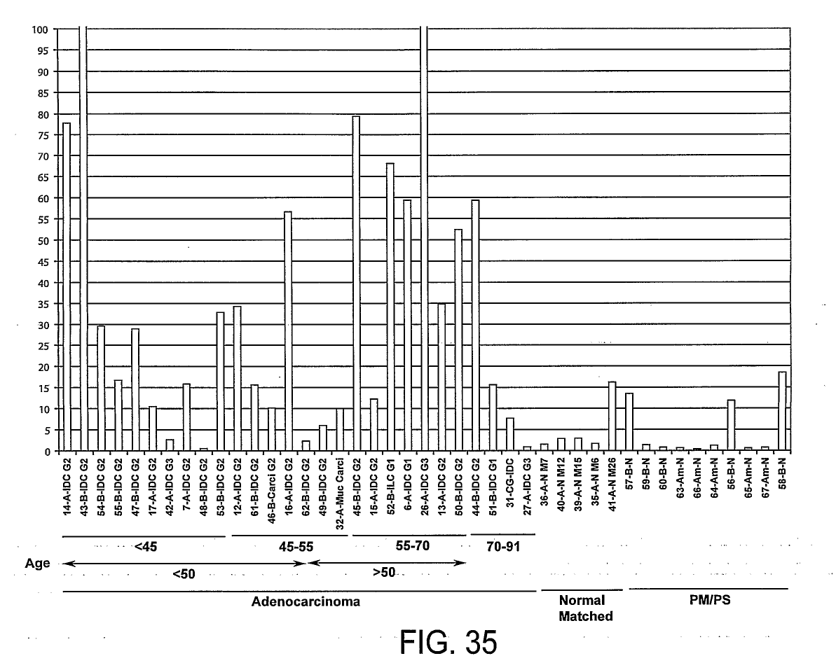

FIG. 35 is a histogram showing over expression of the above-indicated Homo sapiens matrix metalloproteinase 11 (stromelysin 3) (MMP11) transcripts in cancerous breast samples relative to the normal samples.

As is evident from FIG. 35, the expression of Homo sapiens matrix metalloproteinase 11 (stromelysin 3) (MMP11) transcripts detectable by the above amplicon in cancer samples was significantly higher than in the non-cancerous samples (Sample Nos. 46-50, 90-93, 96-99 Table 5, “Tissue samples in breast cancer testing panel”). Notably an over-expression of at least 5 fold was found in 24 out of 28 adenocarcinoma samples.

Statistical analysis was applied to verify the significance of these results, as described below.

The P value for the difference in the expression levels of Homo sapiens matrix metalloproteinase 11 (stromelysin 3) (MMP11) transcripts detectable by the above amplicon(s) in breast cancer samples versus the normal tissue samples was determined by T test as 6.92E-03.

Threshold of 5 fold overexpression was found to differentiate between cancer and normal samples with P value of 2.14E-03 as checked by exact fisher test. The above values demonstrate statistical significance of the results.

By dissecting the patient's population into four age groups (indicated below the graph), we demonstrate that the transcripts detected by the above amplicon are highly expressed mainly in patient with age above 55 and/or patients with an age below 45.

Primer pairs are also optionally and preferably encompassed within the present invention; for example, for the above experiment, the following primer pair was used as a non-limiting illustrative example only of a suitable primer pair: HSSTROL3 junc21-26F (SEQ ID NO:372) forward primer; and HSSTROL3 junc21-26R (SEQ ID NO:371) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 junc21-26 (SEQ ID NO:370).

Primers:

| | |
|---|---|
| Forward primer HSSTROL3 junc21-26F: | (SEQ ID NO: 372) |
| TGGCGCCTCCGTGG | |
| Reverse primer HSSTROL3 junc21-26R: | (SEQ ID NO: 371) |
| ACCCAGTACTGAGCACCTTGG | |
| Amplicon HSSTROL3 junc21-26: | (SEQ ID NO: 370) |

TGGCGCCTCCGTGGGGGCCAGCTGCAGCCCGGCTACCCAGCATTGGCCTCTCGCCACTGGCAG
GGACTGCCCAGCCCTGTGGACGCTGCCTTCGAGGATGCCCAGGGCCACATTTGGTTCTTCCAA
GGTGCTCAGTACTGGGT

Expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in normal and cancerous colon tissues:

Expression of Homo sapiens matrix metalloproteinase 11 (stromelysin 3) (MMP11) transcripts detectable by or according to junc21-26-HSSTROL3 junc21-26 (SEQ ID NO:370) amplicon and primers HSSTROL3 junc21-26F (SEQ ID NO:372) and HSSTROL3 junc21-26R (SEQ ID NO:371) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), G6PD (GenBank Accession No. NM_{—}000402 (SEQ ID NO:8); G6PD amplicon (SEQ ID NO: 44)) and RPS27A (GenBank Accession No. NM_{—}002954 (SEQ ID NO:1); RPS27A amplicon (SEQ ID NO: 47)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 4, above, “Tissue samples in colon cancer testing panel”), to obtain a value of fold up-regulation for each sample relative to median of the normal PM samples.

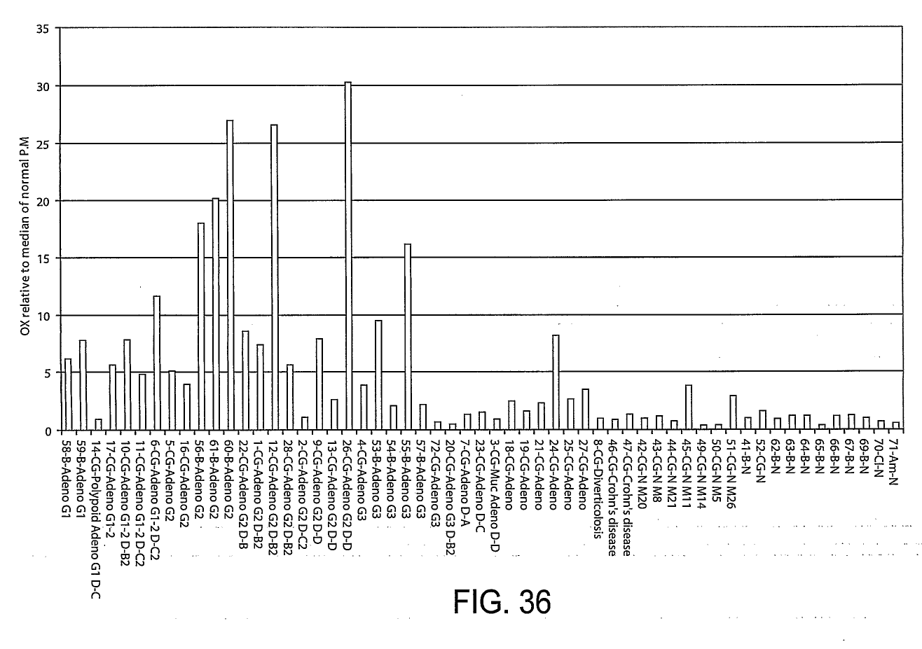

FIG. 36 is a histogram showing over expression of the above-indicated Homo sapiens matrix metalloproteinase 11 (stromelysin 3) (MMP11) transcripts in cancerous colon samples relative to the normal samples.

As is evident from FIG. 36, the expression of Homo sapiens matrix metalloproteinase 11 (stromelysin 3) (MMP11) transcripts detectable by the above amplicon in cancer samples was significantly higher than in the non-cancerous samples (Sample Nos. 41, 52, 62-67, 69-71 Table 3, “Tissue samples in colon cancer testing panel”). Notably an over-expression of at least 5 fold was found in 18 out of 36 adenocarcinoma samples.

Statistical analysis was applied to verify the significance of these results, as described below.

The P value for the difference in the expression levels of Homo sapiens matrix metalloproteinase 11 (stromelysin 3) (MMP11) transcripts detectable by the above amplicon(s) in colon cancer samples versus the normal tissue samples was determined by T test as 2.1E-05.

Threshold of 5 fold overexpression was found to differentiate between cancer and normal samples with P value of 1.99E-03 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: HSSTROL3 junc21-26F (SEQ ID NO:372) forward primer; and HSSTROL3 junc21-26R (SEQ ID NO:371) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 junc21-26 (SEQ ID NO:370).

Primers:

| | |
|---|---|
| Forward primer HSSTROL3 junc21-26F: | (SEQ ID NO: 372) |
| TGGCGCCTCCGTGG | |
| Reverse primer HSSTROL3 junc21-26R: | (SEQ ID NO: 371) |
| ACCCAGTACTGAGCACCTTGG | |
| Amplicon HSSTROL3 junc21-26: | (SEQ ID NO: 370) |

TGGCGCCTCCGTGGGGGCCAGCTGCAGCCCGGCTACCCAGCATTGGCCTCTCGCCACTGGCAG
GGACTGCCCAGCCCTGTGGACGCTGCCTTCGAGGATGCCCAGGGCCACATTTGGTTCTTCCAA
GGTGCTCAGTACTGGGT

Expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in normal and cancerous lung tissues:

Expression of Homo sapiens matrix metalloproteinase 11 (stromelysin 3) (MMP11) transcripts detectable by or according to junc21-26—HSSTROL3 junc21-26 (SEQ ID NO:370) amplicon and primers HSSTROL3 junc21-26F (SEQ ID NO:372) and HSSTROL3 junc21-26R (SEQ ID NO:371) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 5, above, “Tissue samples in lung cancer testing panel”), to obtain a value of fold up-regulation for each sample relative to median of the normal PM samples.

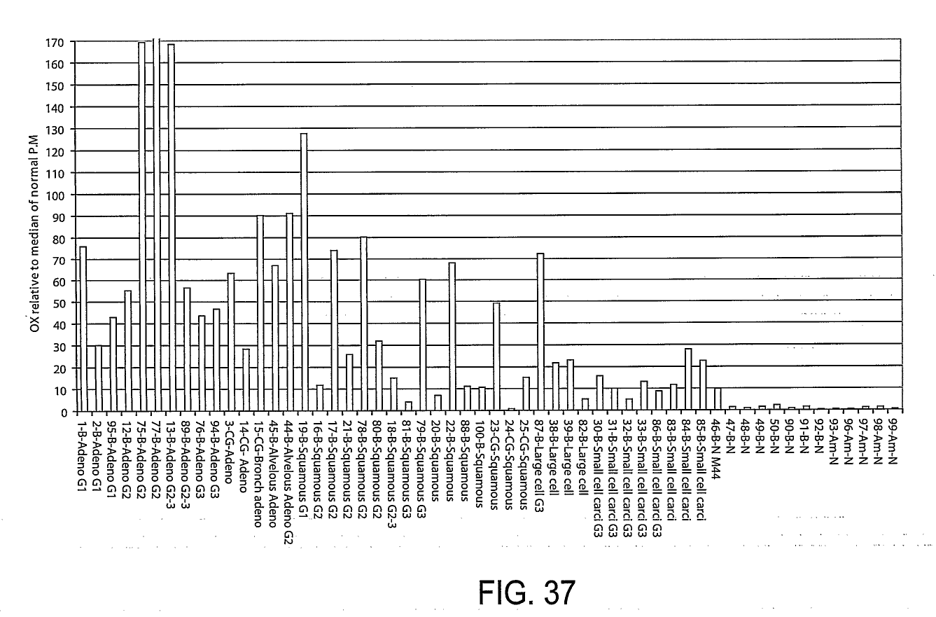

FIG. 37 is a histogram showing over expression of the above-indicated Homo sapiens matrix metalloproteinase 11 (stromelysin 3) (MMP11) transcripts in cancerous lung samples relative to the normal samples.

As is evident from FIG. 37, the expression of Homo sapiens matrix metalloproteinase 11 (stromelysin 3) (MMP11) transcripts detectable by the above amplicon in cancer samples was significantly higher than in the non-cancerous samples (Sample Nos. 46-50, 90-93, 96-99 Table 5, “Tissue samples in lung cancer testing panel”). Notably an over-expression of at least 5 fold was found in 15 out of 15 adenocarcinoma samples, 14 out of 16 squamous cell carcinoma samples, 4 out of 4 large cell carcinoma samples and in 8 out of 8 small cells carcinoma samples.

Statistical analysis was applied to verify the significance of these results, as described below.

The P value for the difference in the expression levels of Homo sapiens matrix metalloproteinase 11 (stromelysin 3) (MMP11) transcripts detectable by the above amplicon in lung cancer samples versus the normal tissue samples was determined by T test as 1.16E-04 in adenocarcinoma, 1.18E-03 in squamous cell carcinoma, 1.31E-01 in large cell carcinoma, 1.62E-03 in small cell.

Threshold of 5 fold overexpression was found to differentiate between cancer and normal samples with P value of 5.75E-08 in adenocarcinoma, 2.99E-06 in squamous cell carcinoma, 5.94E-04 in large cell carcinoma, 1.03E-04 in small cell as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: HSSTROL3 junc21-26F (SEQ ID NO:372) forward primer; and HSSTROL3 junc21-26R (SEQ ID NO:371) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 junc21-26 (SEQ ID NO:370).

Primers:

| | |
|---|---|
| Forward primer HSSTROL3 junc21-26F: | (SEQ ID NO: 372) |
| TGGCGCCTCCGTGG | |
| Reverse primer HSSTROL3 junc21-26R: | (SEQ ID NO: 371) |
| ACCCAGTACTGAGCACCTTGG | |
| Amplicon HSSTROL3 junc21-26: | (SEQ ID NO: 370) |

TGGCGCCTCCGTGGGGGCCAGCTGCAGCCCGGCTACCCAGCATTGGCCTCTCGCCACTGGCAG
GGACTGCCCAGCCCTGTGGACGCTGCCTTCGAGGATGCCCAGGGCCACATTTGGTTCTTCCAA
GGTGCTCAGTACTGGGT

Expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in different normal tissues:

Expression of Homo sapiens matrix metalloproteinase 11 (stromelysin 3) (MMP11) transcripts detectable by or according to HSSTROL3 junc21-26 (SEQ ID NO:370) amplicon and primers: HSSTROL3 junc21-26F (SEQ ID NO:372) and HSSTROL3 junc21-26R (SEQ ID NO:371) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the colon samples (Sample Nos. 1-3 Table 7, “Tissue samples in normal panel”), to obtain a value of relative expression of each sample relative to median of the colon samples.

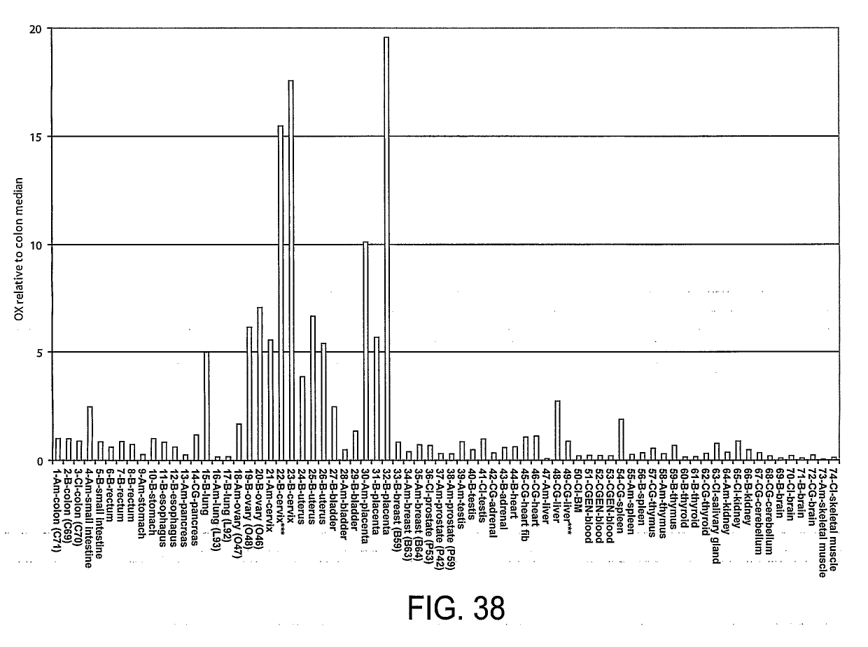

FIG. 38 is a histogram showing expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in different normal tissues.

Primers:

| | |
|---|---|
| Forward primer HSSTROL3 junc21-26F: | (SEQ ID NO: 372) |
| TGGCGCCTCCGTGG | |
| Reverse primer HSSTROL3 junc21-26R: | (SEQ ID NO: 371) |
| ACCCAGTACTGAGCACCTTGG | |
| Amplicon HSSTROL3 junc21-26: | (SEQ ID NO: 370) |

TGGCGCCTCCGTGGGGGCCAGCTGCAGCCCGGCTACCCAGCATTGGCCTCTCGCCACTGGCAG
GGACTGCCCAGCCCTGTGGACGCTGCCTTCGAGGATGCCCAGGGCCACATTTGGTTCTTCCAA
GGTGCTCAGTACTGGGT

Expression of Homo sapiens matrix metalloproteinase 11 (stromelysin 3) (MMP11) HSSTROL3 transcripts which are detectable by amplicon as depicted in sequence name HSSTROL3 junc21-26 (SEQ ID NO:370) in normal and cancerous ovary tissues

Expression of Homo sapiens matrix metalloproteinase 11 (stromelysin 3) (MMP11) transcripts detectable by or according to junc21-26—HSSTROL3 junc21-27 (SEQ ID NO:378) amplicon and primers HSSTROL3 junc21-26F (SEQ ID NO:372) and HSSTROL3 junc21-26R (SEQ ID NO:371) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold up-regulation for each sample relative to median of the normal PM samples.

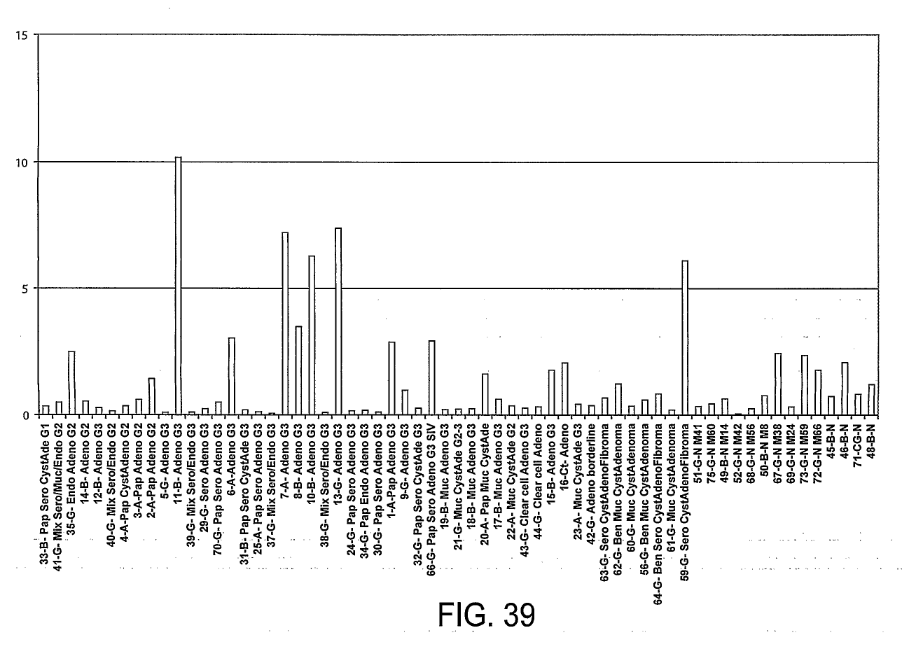

FIG. 39 is a histogram showing over expression of the above-indicated Homo sapiens matrix metalloproteinase 11 (stromelysin 3) (MMP11) transcripts in cancerous ovary samples relative to the normal samples.

As is evident from FIG. 39, the expression of Homo sapiens matrix metalloproteinase 11 (stromelysin 3) (MMP11) transcripts detectable by the above amplicon was higher in a few cancer samples than in the non-cancerous samples (Sample Nos. 45, 46, 48, 71 Table 3 “Tissue samples in ovarian cancer testing panel”). Notably an over-expression of at least 5 fold was found in 4 out of 43 adenocarcinoma samples.

Primer pairs are also optionally and preferably encompassed within the present invention; for example, for the above experiment, the following primer pair was used as a non-limiting illustrative example only of a suitable primer pair: HSSTROL3 junc21-26F (SEQ ID NO:372) forward primer; and HSSTROL3 junc21-26R (SEQ ID NO:371) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 junc21-26 (SEQ ID NO:370).

Primers:

| | |
|---|---|
| Forward primer HSSTROL3 junc21-26F: | (SEQ ID NO: 372) |
| TGGCGCCTCCGTGG | |
| Reverse primer HSSTROL3 junc21-26R: | (SEQ ID NO: 371) |
| ACCCAGTACTGAGCACCTTGG | |
| Amplicon HSSTROL3 junc21-26: | (SEQ ID NO: 370) |

TGGCGCCTCCGTGGGGGCCAGCTGCAGCCCGGCTACCCAGCATTGGCCTCTCGCCACTGGCAG
GGACTGCCCAGCCCTGTGGACGCTGCCTTCGAGGATGCCCAGGGCCACATTTGGTTCTTCCAA
GGTGCTCAGTACTGGGT

Expression of Homo sapiens matrix metalloproteinase 11 (stromelysin 3) (MMP11) HSSTROL3 transcripts which are detectable by amplicon as depicted in sequence name HSSTROL3 junc21-27 (SEQ ID NO:378) in normal and cancerous ovary tissues

Expression of Homo sapiens matrix metalloproteinase 11 (stromelysin 3) (MMP11) transcripts detectable by or according to junc21-27—HSSTROL3 junc21-27 (SEQ ID NO:378) amplicon and primers HSSTROL3 junc21-27F (SEQ ID NO:376) and HSSTROL3 junc21-27R (SEQ ID NO:377) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold up-regulation for each sample relative to median of the normal PM samples.

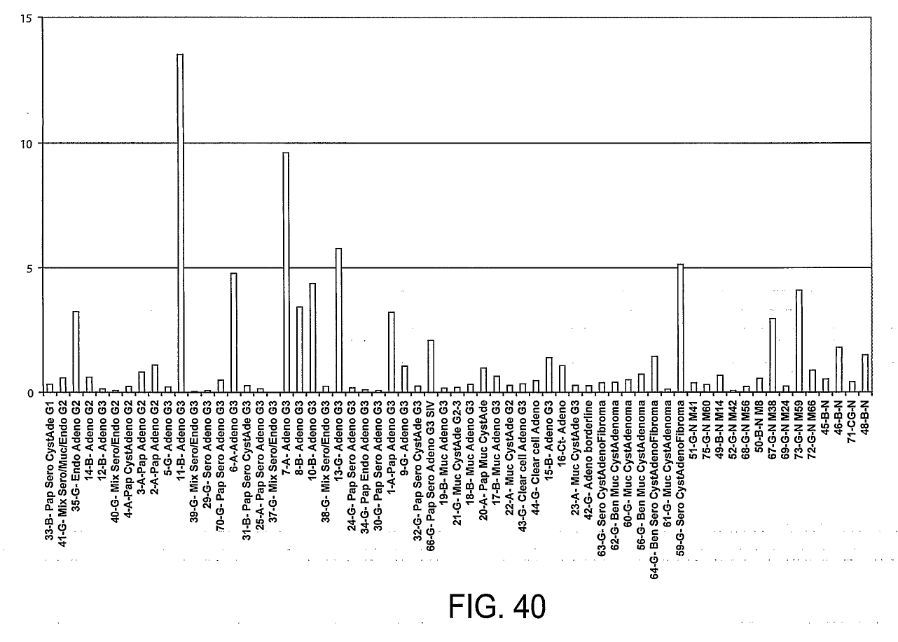

FIG. 40 is a histogram showing over expression of the above-indicated Homo sapiens matrix metalloproteinase 11 (stromelysin 3) (MMP11) transcripts in cancerous ovary samples relative to the normal samples.

As is evident from FIG. 40, the expression of Homo sapiens matrix metalloproteinase 11 (stromelysin 3) (MMP11) transcripts detectable by the above amplicon was higher in a few cancer samples than in the non-cancerous samples (Sample Nos. 45, 46, 48, 71 Table 3, “Tissue samples in ovarian cancer testing panel”). Notably an over-expression of at least 5 fold was found in 3 out of 43 adenocarcinoma samples.

Primer pairs are also optionally and preferably encompassed within the present invention; for example, for the above experiment, the following primer pair was used as a non-limiting illustrative example only of a suitable primer pair: HSSTROL3 junc21-27F (SEQ ID NO:376) forward primer; and HSSTROL3 junc21-27R (SEQ ID NO:377) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 junc21-27 (SEQ ID NO:378).

Primers:

| | |
|---|---|
| Forward primer HSSTROL3 junc21-27F: | (SEQ ID NO: 376) |
| ACATTTGGTTCTTCCAAGGGACTAC | |
| Reverse primer HSSTROL3 junc21-27R: | (SEQ ID NO: 377) |
| TCGATCTCAGAGGGCACCC | |
| Amplicon HSSTROL3 junc21-27: | (SEQ ID NO: 378) |

ACATTTGGTTCTTCCAAGGGACTACTGGCGTTTCCACCCCAGCACCCGGCGTGTAGACAGTCC
CGTGCCCCGCAGGGCCACTGACTGGAGAGGGGTGCCCTCTGAGATCGA

Expression of Stromelysin-3 precursor (SEQ ID NO:363) (EC 3.4.24.-) (Matrix metalloproteinase-11) (MMP-11) (ST3) (SL-3) HSSTROL3 transcripts which are detectable by amplicon as depicted in sequence name HSSTROL3 junc21-27 (SEQ ID NO: 378) in normal and cancerous breast tissues

Expression of Stromelysin-3 precursor (SEQ ID NO:363) transcripts detectable by or according to junc21-27, HSSTROL3 junc21-27 (SEQ ID NO: 378) amplicon and primers HSSTROL3junc21-27F (SEQ ID NO: 376) and HSSTROL3junc21-27R (SEQ ID NO: 377) was measured by real time PCR (RNA was as for the experiment above). In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), G6PD (GenBank Accession No. NM_{—}000402 (SEQ ID NO:8); G6PD amplicon (SEQ ID NO: 44)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67, Table 6, “Tissue samples in breast cancer testing panel”, above), to obtain a value of fold up-regulation for each sample relative to median of the normal PM samples.

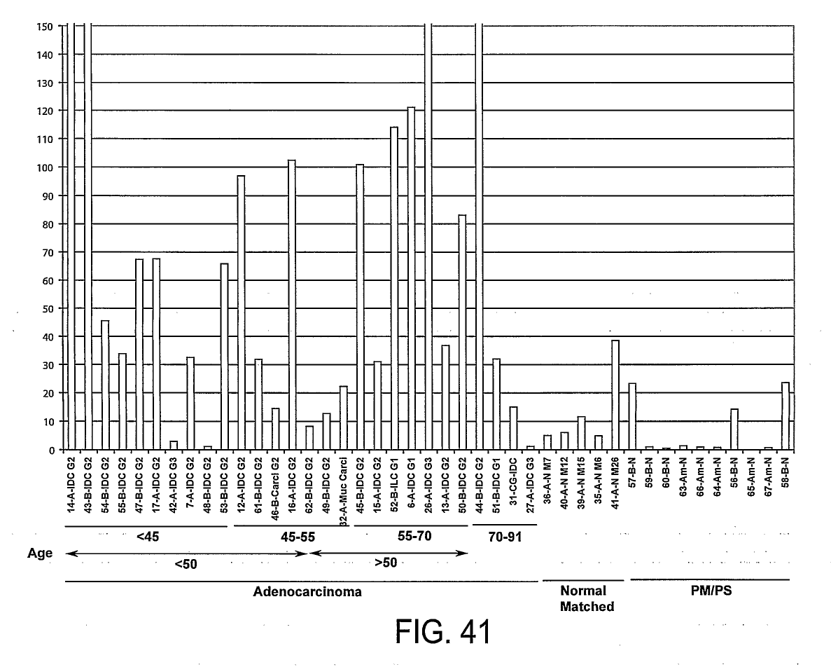

FIG. 41 is a histogram showing over expression of the above-indicated Stromelysin-3 precursor (SEQ ID NO:363) transcripts in cancerous breast samples relative to the normal samples.

As is evident from FIG. 41, the expression of Stromelysin-3 precursor (SEQ ID NO:363) transcripts detectable by the above amplicon in cancer samples was significantly higher than in the non-cancerous samples (Sample Nos. 56-60, 63-67 Table 6, “Tissue samples in breast cancer testing panel”, above). Notably an over-expression of at least 20 fold was found in 21 out of 28 adenocarcinoma samples.

Statistical analysis was applied to verify the significance of these results, as described below.

The P value for the difference in the expression levels of Stromelysin-3 precursor (SEQ ID NO:363) transcripts detectable by the above amplicon in breast cancer samples versus the normal tissue samples was determined by T test as 5.98E-03.

Threshold of 20 fold overexpression was found to differentiate between cancer and normal samples with P value of 3.66E-03 as checked by exact fisher test. The above values demonstrate statistical significance of the results.

By dissecting the patient's population into four age groups (indicated below the graph), we demonstrate that the transcripts detected by the above amplicon are highly expressed mainly in patient with age above 55 and/or patients with an age below 45.

Primer pairs are also optionally and preferably encompassed within the present invention; for example, for the above experiment, the following primer pair was used as a non-limiting illustrative example only of a suitable primer pair: HSSTROL3 junc21-27F (SEQ ID NO:376) forward primer; and HSSTROL3 junc21-27R (SEQ ID NO:377) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 junc21-27 (SEQ ID NO:378).

| | |
|---|---|
| Forward primer HSSTROL3 junc21-27F: | (SEQ ID NO: 376) |
| ACATTTGGTTCTTCCAAGGGACTAC | |
| Reverse primer HSSTROL3 junc21-27R | (SEQ ID NO:377) |
| TCGATCTCAGAGGGCACCC | |
| Amplicon HSSTROL3 junc21-27: | (SEQ ID NO: 378) |

ACATTTGGTTCTTCCAAGGGACTACTGGCGTTTCCACCCCAGCACCCGGCGTGTAGAC
AGTCCCGTGCCCCGCAGGGCCACTGACTGGAGAGGGGTGCCCTCTGAGATCGA

Expression of Homo sapiens matrix metalloproteinase 11 (stromelysin 3) (MMP11) HSSTROL3 transcripts which are detectable by amplicon as depicted in sequence name HSSTROL3 junc21-27 (SEQ ID NO: 378) in normal and cancerous colon tissues

Expression of Homo sapiens matrix metalloproteinase 11 (stromelysin 3) (MMP11) transcripts detectable by or according to junc21-27, HSSTROL3 junc21-27 (SEQ ID NO: 378) amplicon and primers HSSTROL3 junc21-27F (SEQ ID NO: 376) and HSSTROL3 junc21-27R (SEQ ID NO: 377) was measured by real time PCR. In parallel the expression of four housekeeping genes—PBGD (GenBank Accession No. BC019323 (SEQ ID NO:6); amplicon—PBGD-amplicon (SEQ ID NO:32), HPRT1 (GenBank Accession No. NM_{—}000194 (SEQ ID NO:5); amplicon—HPRT1-amplicon (SEQ ID NO:35), G6PD (GenBank Accession No. NM_{—}000402 (SEQ ID NO:8); G6PD-amplicon (SEQ ID NO: 44)), RPS27A (GenBank Accession No. NM_{—}002954 (SEQ ID NO:1); RPS27A amplicon (SEQ ID NO: 47)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 4, above, “Tissue samples in colon cancer testing panel”), to obtain a value of fold up-regulation for each sample relative to median of the normal PM samples.

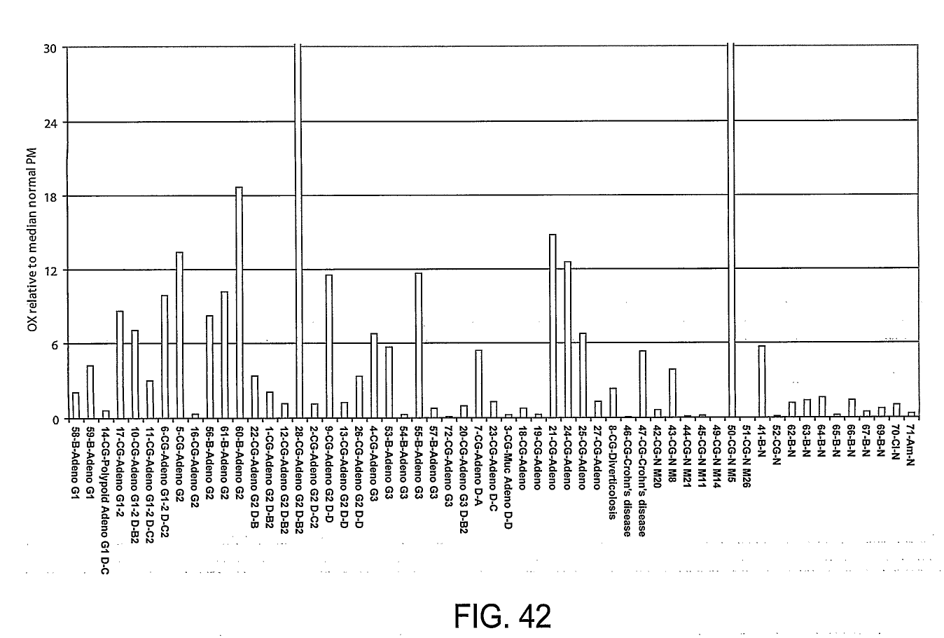

FIG. 42 is a histogram showing over expression of the above-indicated Homo sapiens matrix metalloproteinase 11 (stromelysin 3) (MMP11) transcripts in cancerous colon samples relative to the normal samples.

As is evident from FIG. 42, the expression of Homo sapiens matrix metalloproteinase 11 (stromelysin 3) (MMP11) transcripts detectable by the above amplicon in cancer samples was higher than in the non-cancerous samples (Sample Nos. 41, 52, 62-67, 69-71 Table 4, “Tissue samples in colon cancer testing panel”). Notably an over-expression of at least 6 fold was found in 14 out of 36 adenocarcinoma samples.

Primer pairs are also optionally and preferably encompassed within the present invention; for example, for the above experiment, the following primer pair was used as a non-limiting illustrative example only of a suitable primer pair: HSSTROL3 junc21-27F (SEQ ID NO: 376) forward primer; and HSSTROL3 junc21-27R (SEQ ID NO: 377) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 junc21-27 (SEQ ID NO: 378).

Primers:

Forward primer HSSTROL3 junc21-27F
(SEQ ID NO:376):
ACATTTGGTTCTTCCAAGGGACTAC
Reverse primer HSSTROL3 junc21-27R
(SEQ ID NO:377):
TCGATCTCAGAGGGCACCC
Amplicon HSSTROL3 junc21-27 (SEQ ID NO:378):
ACATTTGGTTCTTCCAAGGGACTACTGGCGTTTCCACCCCAGCACCCGGC
GTGTAGACAGTCCCGTGCCCCGCAGGGCCACTGACTGGAGAGGGGTGCCC
TCTGAGATCGA

Expression of Homo sapiens matrix metalloproteinase 11 (stromelysin 3) (MMP11) HSSTROL3 transcripts which are detectable by amplicon as depicted in sequence name HSSTROL3 junc21-27 (SEQ ID NO: 378) in normal and cancerous lung tissues

Expression of Homo sapiens matrix metalloproteinase 11 (stromelysin 3) (MMP11) transcripts detectable by or according to junc21-27, HSSTROL3 junc21-27 (SEQ ID NO: 378) amplicon and primers HSSTROL3 junc21-27F (SEQ ID NO: 376) and HSSTROL3 junc21-27R (SEQ ID NO: 377) was measured by real time PCR. In parallel the expression of four housekeeping genes—PBGD (GenBank Accession No. BC019323 (SEQ ID NO:6); amplicon—PBGD-amplicon (SEQ ID NO:32),), HPRT1 (GenBank Accession No. NM_{—}000194 (SEQ ID NO:5); amplicon—HPRT1-amplicon (SEQ ID NO:35), Ubiquitin (GenBank Accession No. BC000449 (SEQ ID NO:9); amplicon—Ubiquitin-amplicon (SEQ ID NO:50),) and SDHA (GenBank Accession No. NM_{—}004168 (SEQ ID NO:4); amplicon—SDHA-amplicon (SEQ ID NO:29),), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 5, above, “Tissue samples in lung cancer testing panel”), to obtain a value of fold up-regulation for each sample relative to median of the normal PM samples.

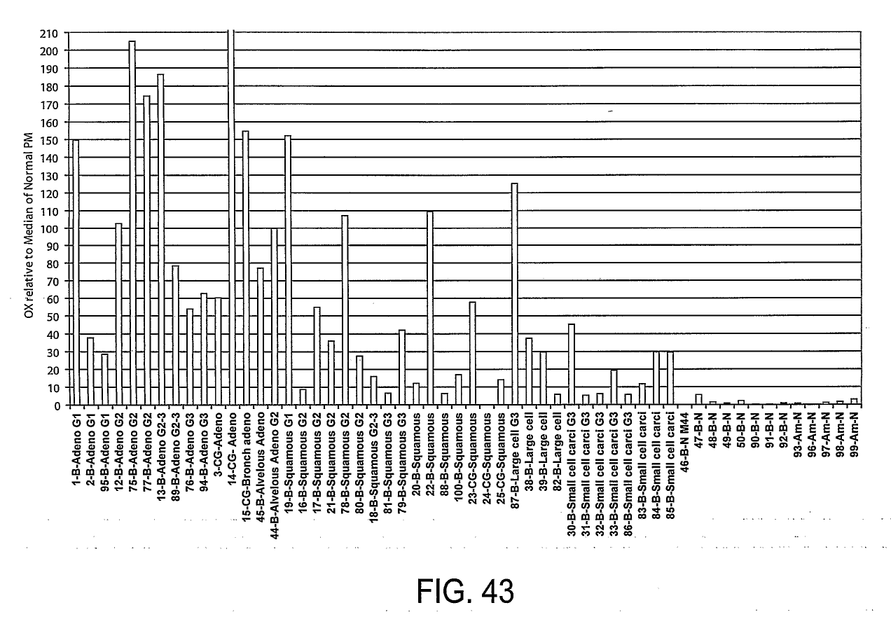

FIG. 43 is a histogram showing over expression of the above-indicated Homo sapiens matrix metalloproteinase 11 (stromelysin 3) (MMP11) transcripts in cancerous lung samples relative to the normal samples.

As is evident from FIG. 43, the expression of Homo sapiens matrix metalloproteinase 11 (stromelysin 3) (MMP11) transcripts detectable by the above amplicon in cancer samples was significantly higher than in the non-cancerous samples (Sample Nos. 46-50, 90-93, 96-99 Table 5, “Tissue samples in lung cancer testing panel”). Notably an over-expression of at least 10 fold was found in 15 out of 15 adenocarcinoma samples, 13 out of 16 squamous cell carcinoma samples, 3 out of 4 large cell carcinoma samples and in 5 out of 8 small cells carcinoma samples.

Primer pairs are also optionally and preferably encompassed within the present invention; for example, for the above experiment, the following primer pair was used as a non-limiting illustrative example only of a suitable primer pair: HSSTROL3 junc21-27F (SEQ ID NO: 376) forward primer; and HSSTROL3 junc21-27R (SEQ ID NO: 377) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 junc21-27 (SEQ ID NO: 378).

Primers:

Forward primer HSSTROL3 junc21-27F
(SEQ ID NO:376):
ACATTTGGTTCTTCCAAGGGACTAC
Reverse primer HSSTROL3 junc21-27R
(SEQ ID NO:377):
TCGATCTCAGAGGGCACCC
Amplicon HSSTROL3 junc21-27 (SEQ ID NO:378):
ACATTTGGTTCTTCCAAGGGACTACTGGCGTTTCCACCCCAGCACCCGGC
GTGTAGACAGTCCCGTGCCCCGCAGGGCCACTGACTGGAGAGGGGTGCCC
TCTGAGATCGA

Expression of Stromelysin-3 precursor (SEQ ID NO:363) (EC 3.4.24.-) (Matrix metalloproteinase-11) (MMP-11) (ST3) (SL-3)HSSTROL3 transcripts which are detectable by amplicon as depicted in sequence name HSSTROL3 junc21-27 (SEQ ID NO: 378) in different normal tissues

Expression of Stromelysin-3 precursor (SEQ ID NO:363) (EC 3.4.24.-) (Matrix metalloproteinase-11) (MMP-11) (ST3) (SL-3) transcripts detectable by or according to HSSTROL3 junc21-27 (SEQ ID NO: 378) amplicon and HSSTROL3 junc21-27 F (SEQ ID NO: 376) and HSSTROL3 junc21-27 R (SEQ ID NO: 377) was measured by real time PCR. In parallel the expression of four housekeeping genes UBC (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)) was measured similarly. For each RT sample, the expression of the above amplicon was normalized to the geometric mean of the quantities of the housekeeping genes. The normalized quantity of each RT sample was then divided by the median of the quantities of the breast samples (sample Nos. 33-35 Table 7, “Tissue samples in normal panel”), to obtain a value of relative expression of each sample relative to median of the breast samples.

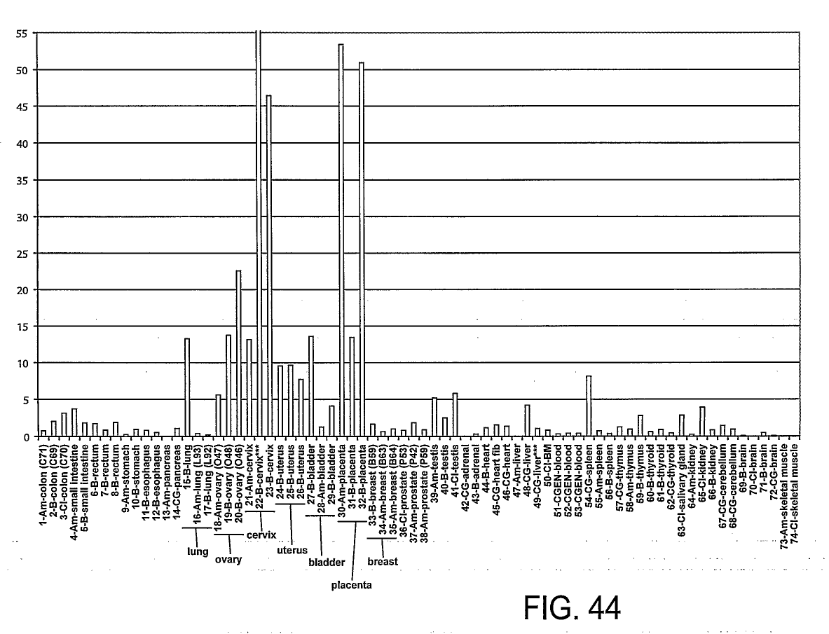

The results are presented in FIG. 44, demonstrating the expression of Stromelysin-3 precursor (SEQ ID NO:363) (EC 3.4.24.-) (Matrix metalloproteinase-11) (MMP-11) (ST3) (SL-3) HSSTROL3 transcripts, which are detectable by amplicon as depicted in sequence name HSSTROL3 junc21-27 (SEQ ID NO: 378), in different normal tissues.

Primers:

Forward primer HSSTROL3 junc21-27F
(SEQ ID NO:376):
ACATTTGGTTCTTCCAAGGGACTAC
Reverse primer HSSTROL3 junc21-27R
(SEQ ID NO:377):
TCGATCTCAGAGGGCACCC
Amplicon HSSTROL3 junc21-27 (SEQ ID NO:378):
ACATTTGGTTCTTCCAAGGGACTACTGGCGTTTCCACCCCAGCACCCGGC
GTGTAGACAGTCCCGTGCCCCGCAGGGCCACTGACTGGAGAGGGGTGCCC
TCTGAGATCGA

Expression of Homo sapiens matrix metalloproteinase 11 (stromelysin 3) (MMP11) HSSTROL3 transcripts which are detectable by amplicon as depicted in sequence name HSSTROL3 seg20-21 (SEQ ID NO:381) in normal and cancerous colon tissues

Expression of Homo sapiens matrix metalloproteinase 11 (stromelysin 3) (MMP11) transcripts detectable by or according to seg20-21 HSSTROL3 seg20-21 (SEQ ID NO:381) amplicon and primers HSSTROL3 seg20-21F (SEQ ID NO:379) and HSSTROL3 seg20-21R (SEQ ID NO:380) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), G6PD (GenBank Accession No. NM_{—}000402 (SEQ ID NO:8); G6PD amplicon (SEQ ID NO: 44)) and RPS27A (GenBank Accession No. NM_{—}002954 (SEQ ID NO:1); RPS27A amplicon (SEQ ID NO: 47)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 4, above, “Tissue samples in colon cancer testing panel”), to obtain a value of fold up-regulation for each sample relative to median of the normal PM samples.

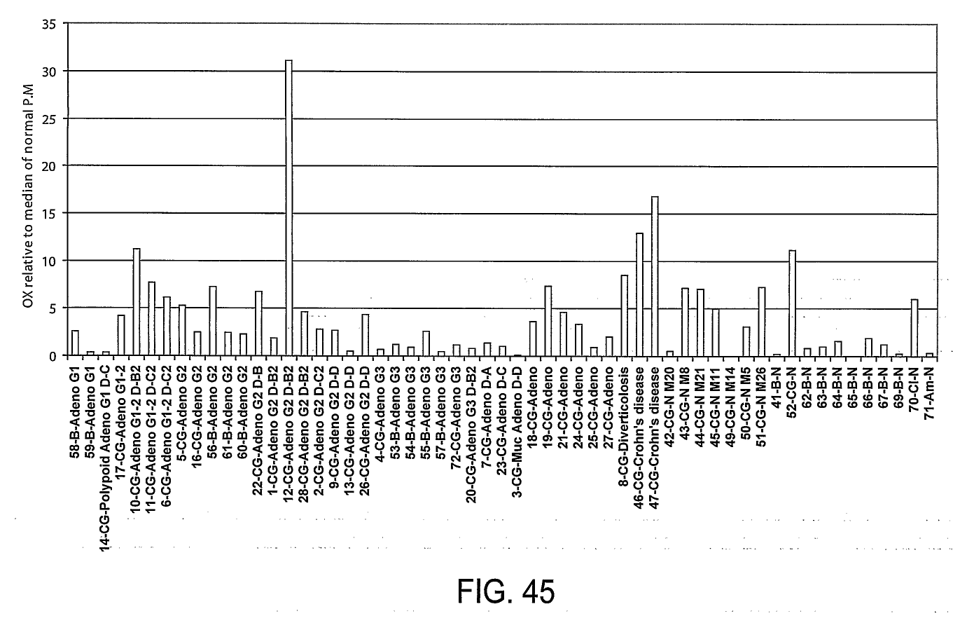

FIG. 45 is a histogram showing over expression of the above-indicated Homo sapiens matrix metalloproteinase 11 (stromelysin 3) (MMP11) transcripts in cancerous colon samples relative to the normal samples.

As is evident from FIG. 45, the expression of Homo sapiens matrix metalloproteinase 11 (stromelysin 3) (MMP11) transcripts detectable by the above amplicon was higher in a few cancer samples than in the non-cancerous samples (Sample Nos. 41, 52, 62-67, 69-71 Table 4, “Tissue samples in colon cancer testing panel”). Notably an over-expression of at least 5 fold was found in 8 out of 36 adenocarcinoma samples.

Primer pairs are also optionally and preferably encompassed within the present invention; for example, for the above experiment, the following primer pair was used as a non-limiting illustrative example only of a suitable primer pair: HSSTROL3 seg20-21F (SEQ ID NO:379) forward primer; and HSSTROL3 seg20-21R (SEQ ID NO:380) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 seg20-21 (SEQ ID NO:381).

Primers:

Forward primer HSSTROL3 seg20-21F
(SEQ ID NO:379):
TCTGCTGGCCACTGTGACTG
Reverse primer HSSTROL3 seg20-21R
(SEQ ID NO:380):
GAAGAAAAAGAGCTCGCCTCG
Amplicon HSSTROL3 seg20-21 (SEQ ID NO:381):
TCTGCTGGCCACTGTGACTGCAGCATATGCCCTCAGCATGTGTCCCTCTC
TCCCACCCCAGCCAGACGCCCCGCCAGATGCCTGTGAGGCCTCCTTTGAC
GCGGTCTCCACCATCCGAGGCGAGCTCTTTTTCTTC

Expression of Homo sapiens matrix metalloproteinase 11 (stromelysin 3) (MMP11) HSSTROL3 transcripts which are detectable by amplicon as depicted in sequence name HSSTROL3 seg20-21 (SEQ ID NO: 381) in normal and cancerous ovary tissues

Expression of Homo sapiens matrix metalloproteinase 11 (stromelysin 3) (MMP11) transcripts detectable by or according to seg20-21—HSSTROL3 seg20-21 (SEQ ID NO: 381) amplicon and primers HSSTROL3 seg20-21F (SEQ ID NO:379) and HSSTROL3 seg20-21R (SEQ ID NO: 380) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold up-regulation for each sample relative to median of the normal PM samples.

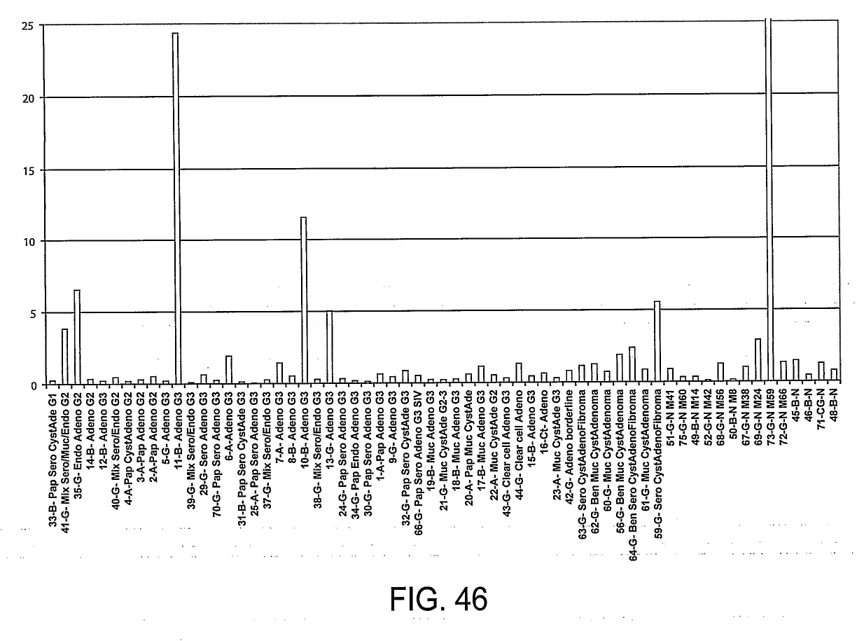

FIG. 46 is a histogram showing over expression of the above-indicated Homo sapiens matrix metalloproteinase 11 (stromelysin 3) (MMP11) transcripts in cancerous ovary samples relative to the normal samples.

As is evident from FIG. 46 the expression of Homo sapiens matrix metalloproteinase 11 (stromelysin 3) (MMP11) transcripts detectable by the above amplicon was higher in a few cancer samples than in the non-cancerous samples (Sample Nos. 45, 46, 48, 71 Table 3, “Tissue samples in ovarian cancer testing panel”). Notably an over-expression of at least 5 fold was found in 4 out of 43 adenocarcinoma samples.

Primer pairs are also optionally and preferably encompassed within the present invention; for example, for the above experiment, the following primer pair was used as a non-limiting illustrative example only of a suitable primer pair: HSSTROL3 seg20-21F (SEQ ID NO: 379) forward primer; and HSSTROL seg20-21R (SEQ ID NO: 380) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL seg20-21 (SEQ ID NO: 381).

Primers:

Forward primer HSSTROL seg20-21F
(SEQ ID NO:379):
TCTGCTGGCCACTGTGACTG
Reverse primer HSSTROL seg20-21R
(SEQ ID NO:380):
GAAGAAAAAGAGCTCGCCTCG
Amplicon HSSTROL seg20-21 (SEQ ID NO:381):
TCTGCTGGCCACTGTGACTGCAGCATATGCCCTCAGCATGTGTCCCTCTC
TCCCACCCCAGCCAGACGCCCCGCCAGATGCCTGTGAGGCCTCCTTTGAC
GCGGTCTCCACCATCCGAGGCGAGCTCTTTTTCTTC

Expression of Stromelysin-3 precursor (SEQ ID NO:363) (EC 3.4.24.) (Matrix metalloproteinase-11) (MMP-11) (ST3) (SL-3HSSTROL3) transcripts which are detectable by amplicon as depicted in sequence name HSSTROL3 seg20-21 (SEQ ID NO:381) in normal and cancerous Prostate tissues

Expression of Stromelysin-3 precursor (SEQ ID NO:363) (EC 3.4.24.-) (Matrix metalloproteinase-11) (MMP-11) (ST3) (SL-3) transcripts detectable by or according to seg20-21, HSSTROL3 seg20-21 (SEQ ID NO:381) amplicon and HSSTROL3 seg20-21F (SEQ ID NO:379) and HSSTROL3 seg20-21R (SEQ ID NO:380) primers was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and RPL19 (GenBank Accession No. NM_{—}000981 (SEQ ID NO:7); RPL19 amplicon (SEQ ID NO: 38)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 42, 48-53, 59-63, Table 2, “Tissue samples in prostate cancer testing panel”, above), to obtain a value of fold up-regulation for each sample relative to median of the normal PM samples.

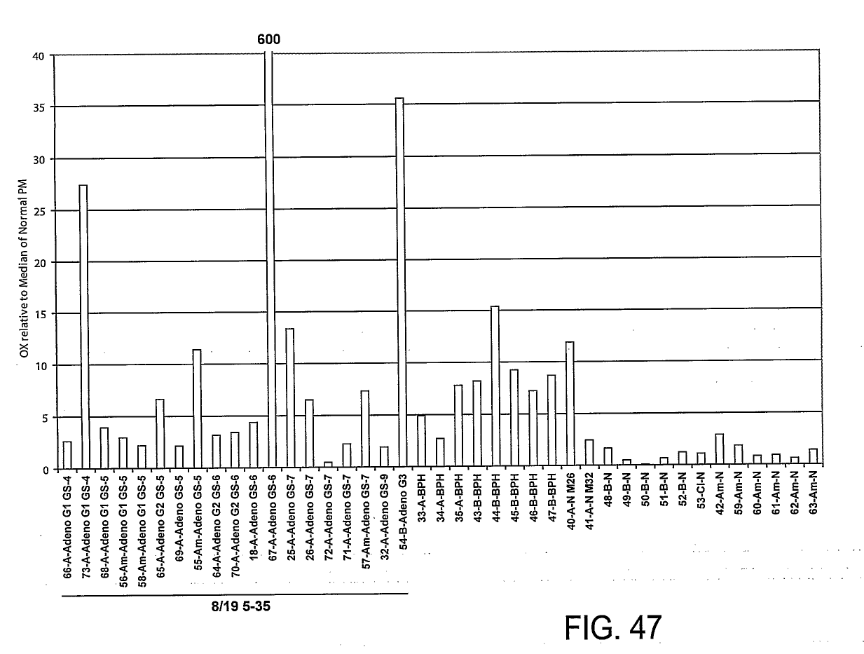

FIG. 47 is a histogram showing over expression of the above-indicated Stromelysin-3 precursor (SEQ ID NO:363) transcripts in cancerous Prostate samples relative to the normal samples. Values represent the average of duplicate experiments.

As is evident from FIG. 47, the expression of Stromelysin-3 precursor (SEQ ID NO:363) transcripts detectable by the above amplicon in cancer samples was higher than in several non-cancerous samples (Sample Nos. 42, 48-53, 59-63, Table 2, “Tissue samples in prostate cancer testing panel”). Notably an over-expression of at least 5 fold was found in 8 out of 19 adenocarcinoma samples.

Primer pairs are also optionally and preferably encompassed within the present invention; for example, for the above experiment, the following primer pair was used as a non-limiting illustrative example only of a suitable primer pair: HSSTROL3 seg20-21F (SEQ ID NO:379) forward primer; and HSSTROL3 seg20-21R (SEQ ID NO:380) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 seg20-21 (SEQ ID NO: 381).

Forward primer HSSTROL3 seg20-21F
(SEQ ID NO:379):
TCTGCTGGCCACTGTGACTG
Reverse primer HSSTROL3 seg20-21R
(SEQ ID NO:380):
GAAGAAAAAGAGCTCGCCTCG
Amplicon HSSTROL3 seg20-21 (SEQ ID NO:381):
TCTGCTGGCCACTGTGACTGCAGCATATGCCCTCAGCATGTGTCCCTCTC
TCCCACCCCAGCCAGACGCCCCGCCAGATGCCTGTGAGGCCTCCTTTGAC
GCGGTCTCCACCATCCGAGGCGAGCTCTTTTTCTTC

Expression of Homo sapiens matrix metalloproteinase 11 (stromelysin 3) (MMP11) HSSTROL3 transcripts which are detectable by amplicon as depicted in sequence name HSSTROL3 seg20-21 (SEQ ID NO:381) in normal and cancerous lung tissues

Expression of Homo sapiens matrix metalloproteinase 11 (stromelysin 3) (MMP11) transcripts detectable by or according to seg20-21, HSSTROL3 seg20-21 (SEQ ID NO:381) amplicon and primers HSSTROL3 seg20-21F (SEQ ID NO:379) and HSSTROL3 seg20-21R (SEQ ID NO:380) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 5, above, “Tissue samples in lung cancer testing panel”), to obtain a value of fold up-regulation for each sample relative to median of the normal PM samples.

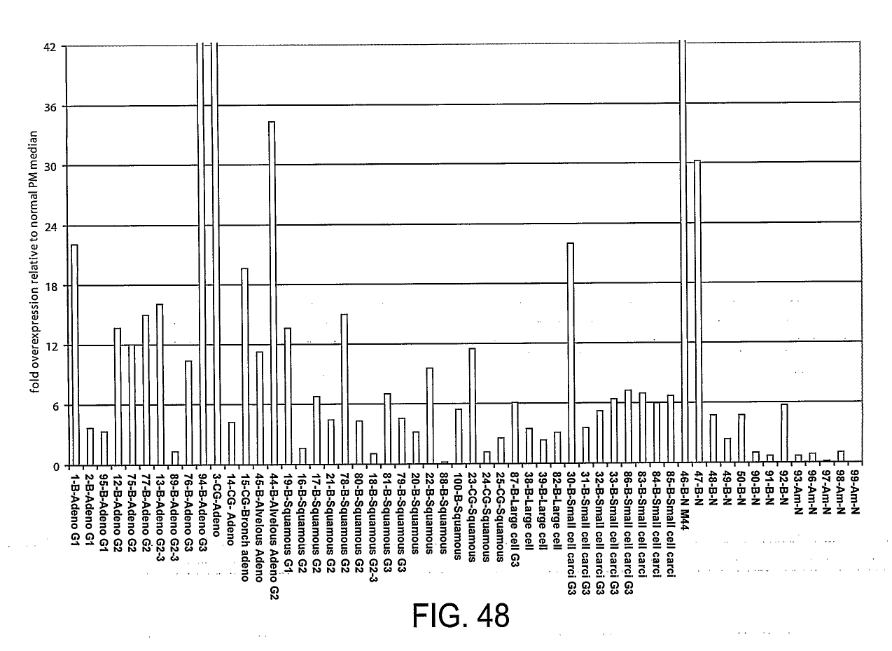

FIG. 48 is a histogram showing over expression of the above-indicated Homo sapiens matrix metalloproteinase 11 (stromelysin 3) (MMP11) transcripts in cancerous lung samples relative to the normal samples.

As is evident from FIG. 48, the expression of Homo sapiens matrix metalloproteinase 11 (stromelysin 3) (MMP11) transcripts detectable by the above amplicon in cancer samples was significantly higher than in the non-cancerous samples (Sample Nos. 46-50, 90-93, 96-99 Table 5, above, “Tissue samples in lung cancer testing panel”). Notably an over-expression of at least 6 fold was found in 11 out of 15 adenocarcinoma samples, 6 out of 16 squamous cell carcinoma samples, 1 out of 4 large cell carcinoma samples and in 6 out of 8 small cells carcinoma samples.

Primer pairs are also optionally and preferably encompassed within the present invention; for example, for the above experiment, the following primer pair was used as a non-limiting illustrative example only of a suitable primer pair: HSSTROL3 seg20-21F (SEQ ID NO:379) forward primer; and HSSTROL3 seg20-21R (SEQ ID NO:380) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 seg20-21 (SEQ ID NO:381).

Primers:

Forward primer HSSTROL3 seg20-21F
(SEQ ID NO:379):
TCTGCTGGCCACTGTGACTG
Reverse primer HSSTROL3 seg20-21R
(SEQ ID NO:380):
GAAGAAAAAGAGCTCGCCTCG
Amplicon HSSTROL3 seg20-21 (SEQ ID NO:381):
TCTGCTGGCCACTGTGACTGCAGCATATGCCCTCAGCATGTGTCCCTCTC
TCCCACCCCAGCCAGACGCCCCGCCAGATGCCTGTGAGGCCTCCTTTGAC
GCGGTCTCCACCATCCGAGGCGAGCTC

Expression of Stromelysin-3 precursor (SEQ ID NO:363) (EC 3.4.24.-) (Matrix metalloproteinase-11) (MMP-11) (ST3) (SL-3) HSSTROL3 transcripts which are detectable by amplicon as depicted in sequence name HSSTROL3 junc20-21 in normal and cancerous breast tissues

Expression of Stromelysin-3 precursor (SEQ ID NO:363) transcripts detectable by or according to junc20-21, HSSTROL3junc20-21 (SEQ ID NO:698) amplicon and primers HSSTROL3junc20-21F (SEQ ID NO:699) and HSSTROL3junc20-21R (SEQ ID NO:670) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), G6PD (GenBank Accession No. NM_{—}000402 (SEQ ID NO:8); G6PD amplicon (SEQ ID NO: 44)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67, Table 6, “Tissue samples in breast cancer testing panel”, above), to obtain a value of fold up-regulation for each sample relative to median of the normal PM samples.

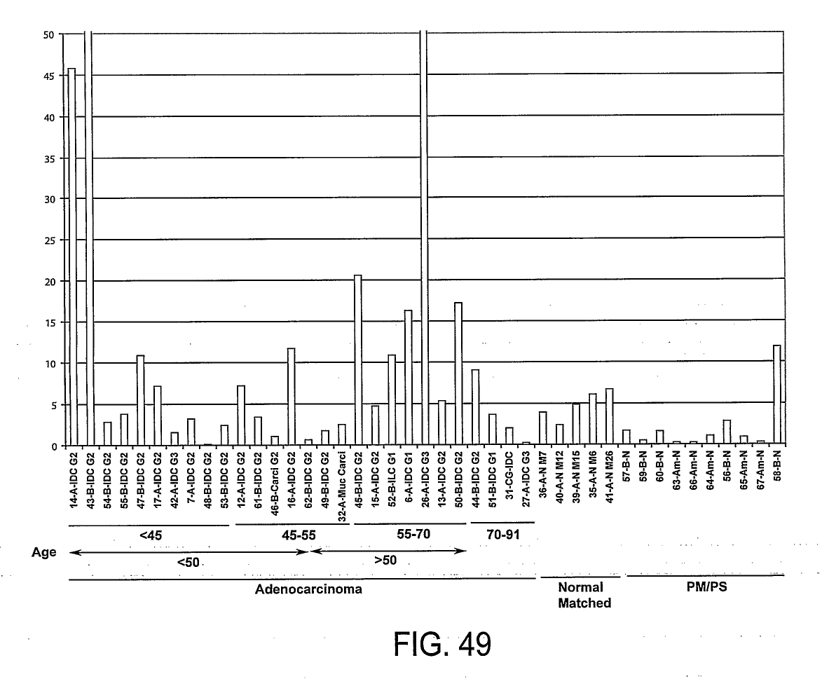

FIG. 49 is a histogram showing over expression of the above-indicated Stromelysin-3 precursor (SEQ ID NO:363) transcripts in cancerous breast samples relative to the normal samples.

As is evident from FIG. 49, the expression of Stromelysin-3 precursor (SEQ ID NO:363) transcripts detectable by the above amplicon in cancer samples was significantly higher than in the non-cancerous samples (Sample Nos. 56-60, 63-67, Table 6, “Tissue samples in breast cancer testing panel”). Notably an over-expression of at least 5 fold was found in 13 out of 28 adenocarcinoma samples.

Statistical analysis was applied to verify the significance of these results, as described below.

The P value for the difference in the expression levels of Stromelysin-3 precursor (SEQ ID NO:363) transcripts detectable by the above amplicon in breast cancer samples versus the normal tissue samples was determined by T test as 1.28E-02.

Threshold of 5 fold overexpression was found to differentiate between cancer and normal samples with P value of 4.26E-02 as checked by exact fisher test. The above values demonstrate statistical significance of the results.

By dissecting the patient's population into four age groups (indicated below the graph), we demonstrate that the transcripts detected by the above amplicon are highly expressed mainly in patients with age above 55 and/or patients with an age below 45.

Primer pairs are also optionally and preferably encompassed within the present invention; for example, for the above experiment, the following primer pair was used as a non-limiting illustrative example only of a suitable primer pair: HSSTROL3junc20-21F (SEQ ID NO:699) forward primer; and HSSTROL3junc20-21R (SEQ ID NO:670) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 junc20-21 (SEQ ID NO:698).

Forward primer HSSTROL3 junc20-21F
(SEQ ID NO:699):
TCTGCTGGCCACTGTGACTG
Reverse primer HSSTROL3 junc20-21R
(SEQ ID NO:670):
GAAGAAAAAGAGCTCGCCTCG
Amplicon HSSTROL3junc20-21 (SEQ ID NO:698):
TCTGCTGGCCACTGTGACTGCAGCATATGCCCTCAGCATGTGTCCCTCTC
TCCCACCCCAGCCAGACGCCCCGCCAGATGCCTGTGAGGCCTCCTTTGAC
GCGGTCTCCACCATCCGAGGCGAGCTCTTTTTCTTC

Expression of Homo sapiens matrix metalloproteinase 11 (stromelysin 3) (MMP11) HSSTROL3 transcripts which are detectable by amplicon as depicted in sequence name HSSTROL3 seg24 (SEQ ID NO:384) in normal and cancerous ovary tissues

Expression of Homo sapiens matrix metalloproteinase 11 (stromelysin 3) (MMP11) transcripts detectable by or according to seg24, HSSTROL3 seg24 (SEQ ID NO:384) amplicon and primers HSSTROL3 seg24F (SEQ ID NO:382) and HSSTROL3 seg24R (SEQ ID NO:383) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold differential expression for each sample relative to median of the normal PM samples.

In one experiment that was carried out no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: HSSTROL3 seg24F (SEQ ID NO:382) forward primer; and HSSTROL3 seg24R (SEQ ID NO:383) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 seg24 (SEQ ID NO:384).

Primers:

Forward primer HSSTROL3 seg24F (SEQ ID NO:382):
ATTTCCATCCTCAACTGGCAGA
Reverse primer HSSTROL3 seg24R (SEQ ID NO:383):
TGCCCTGGAACCCACG
Amplicon HSSTROL3 seg24 (SEQ ID NO:384):
ATTTCCATCCTCAACTGGCAGAGATGAGAGCCTGGAGCATTGCAGATGCC
AGGGACTTCACAAATGAAGGCACAGCATGGGAAACCTGCGTGGGTTCCAG
GGCA

Expression of Stromelysin-3 precursor (SEQ ID NO:363) (EC 3.4.24.-) (Matrix metalloproteinase-11) (MMP-11) (ST3) SL-3 HSSTROL3 transcripts which are detectable by amplicon as depicted in sequence name HSSTROL3 seg24 (SEQ ID NO:384) in normal and cancerous breast tissues

Expression of Stromelysin-3 precursor (SEQ ID NO:363) (EC 3.4.24.-) (Matrix metalloproteinase-11) (MMP-1) (ST3) (SL-3 transcripts detectable by or according to seg24 HSSTROL3 seg24 (SEQ ID NO:384) amplicon) and HSSTROL3 seg24F (SEQ ID NO:382) and HSSTROL3 seg24R (SEQ ID NO:383) primers was measured by real time PCR. In parallel the expression of four housekeeping genes PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)) SDHA (GenBank Accession No. NM_{—}004168 (SEQ ID NO:4); amplicon—SDHA-amplicon (SEQ ID NO:29)) and G6PD (GenBank Accession No. NM_{—}000402 (SEQ ID NO:8); G6PD amplicon (SEQ ID NO: 44)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67, Table 6, above, “Tissue samples inbreast cancer testing panel”), to obtain a value of fold up-regulation for each sample relative to median of the normal PM samples.

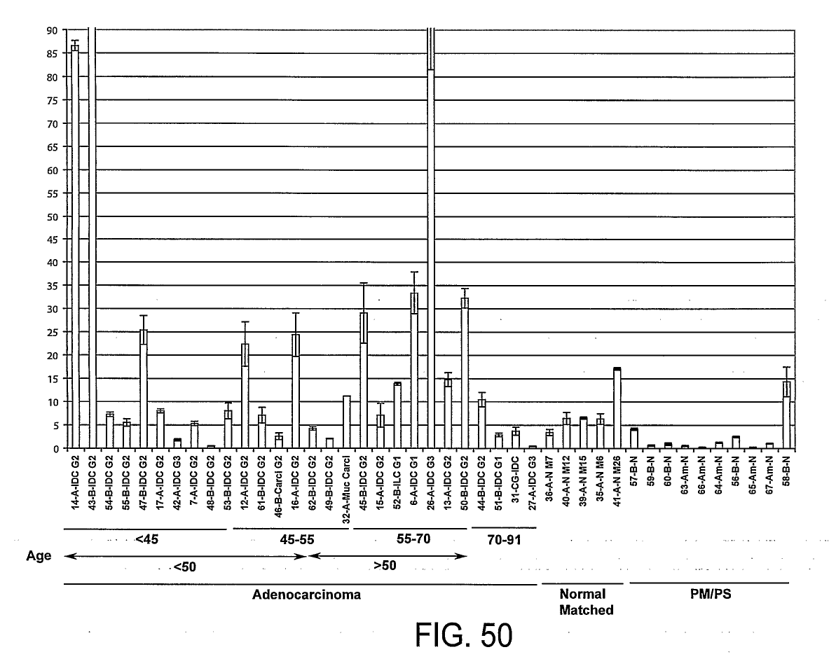

FIG. 50 is a histogram showing over expression of the above-indicated Stromelysin-3 precursor (SEQ ID NO:363) (EC 3.4.24.-) (Matrix metalloproteinase-11) (MMP-11) (ST3) (SL-3) transcripts in cancerous breast samples relative to the normal samples. Values represent the average of duplicate experiments. Error bars indicate the minimal and maximal values obtained.

As is evident from FIG. 50, the expression of Stromelysin-3 precursor (SEQ ID NO:363) (EC 3.4.24.-) (Matrix metalloproteinase-11) (MMP-11) (ST3) (SL-3) transcripts detectable by the above amplicon in cancer samples was significantly higher than in the non-cancerous samples (Sample Nos. 56-60, 63-67 Table 6, above, “Tissue samples inbreast cancer testing panel”). Notably an over-expression of at least 5 fold was found in 20 out of 28 adenocarcinoma samples.

Statistical analysis was applied to verify the significance of these results, as described below.

The P value for the difference in the expression levels of Stromelysin-3 precursor (SEQ ID NO:363) (EC 3.4.24.-) (Matrix metalloproteinase-11) (MMP-11) (ST3) (SL-3) transcripts detectable by the above amplicon in Breast cancer samples versus the normal tissue samples was determined by T test as 6.46E-03.

Threshold of 5 fold overexpression was found to differentiate between cancer and normal samples with P value of 1.12E-03 as checked by exact fisher test. The above values demonstrate statistical significance of the results.

By dissecting the patient's population into four age groups (indicated below the graph), we demonstrate that the transcripts detected by the above amplicon are highly expressed mainly in patients with age above 55 and/or patients with an age below 45

Primer pairs are also optionally and preferably encompassed within the present invention; for example, for the above experiment, the following primer pair was used as a non-limiting illustrative example only of a suitable primer pair: HSSTROL3 seg24F (SEQ ID NO:382) forward primer; and HSSTROL3 seg24R (SEQ ID NO:383) reverse primer. The present invention also preferably encompasses any amplicon obtained through the use of any suitable primer pair; for example, for the above experiment, the following amplicon was obtained as a non-limiting illustrative example only of a suitable amplicon: HSSTROL3 seg24 (SEQ ID NO:384).

HSSTROL3 seg24 Forward Primer (SEQ ID NO:382):
ATTTCCATCCTCAACTGGCAGA
HSSTROL3 seg24 Reverse Primer (SEQ ID NO:383):
TGCCCTGGAACCCACG
HSSTROL3 seg24 Amplicon (SEQ ID NO:384):
ATTTCCATCCTCAACTGGCAGAGATGAGAGCCTGGAGCATTGCAGATGCC
AGGGACTTCACAAATGAAGGCACAGCATGGGAAACCTGCGTGGGTTCCAG
GGCA

Expression of Homo sapiens matrix metalloproteinase 11 (stromelysin 3) (MMP11) HSSTROL3 transcripts which are detectable by amplicon as depicted in sequence name HSSTROL3 seg24 (SEQ ID NO:384) in normal and cancerous colon tissues

Expression of Homo sapiens matrix metalloproteinase 11 (stromelysin 3) (MMP11) transcripts detectable by or according to seg24, HSSTROL3 seg24 (SEQ ID NO:384) amplicon and primers HSSTROL3 seg24F (SEQ ID NO:382) and HSSTROL3 seg24R (SEQ ID NO:383) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), G6PD (GenBank Accession No. NM_{—}000402 (SEQ ID NO:8); G6PD amplicon (SEQ ID NO: 44),), RPS27A (GenBank Accession No. NM_{—}002954 (SEQ ID NO:1); RPS27A amplicon (SEQ ID NO: 47)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41,52,62-67, 69-71, Table 4, above, “Tissue samples in colon cancer testing panel”), to obtain a value of fold differential expression for each sample relative to median of the normal PM samples.

In one experiment that was carried out no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: HSSTROL3 seg24F (SEQ ID NO:382) forward primer; and HSSTROL3 seg24R (SEQ ID NO:383) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 seg24 (SEQ ID NO:384).

Primers:

Forward primer HSSTROL3 seg24F (SEQ ID NO:382):
ATTTCCATCCTCAACTGGCAGA
Reverse primer HSSTROL3 seg24R (SEQ ID NO:383):
TGCCCTGGAACCCACG
Amplicon HSSTROL3 seg24 (SEQ ID NO:384):
ATTTCCATCCTCAACTGGCAGAGATGAGAGCCTGGAGCATTGCAGATGCC
AGGGACTTCACAAATGAAGGCACAGCATGGGAAACCTGCGTGGGTTCCAG
GGCA

Expression of Stromelysin-3 precursor (SEQ ID NO:363) HSSTROL3 transcripts which are detectable by amplicon as depicted in sequence name HSSTROL3 seg24 (SEQ ID NO:384) in normal and cancerous lung tissues

Expression of Stromelysin-3 precursor (SEQ ID NO:363) (EC 3.4.24.-) (Matrix metalloproteinase-11) (MMP-11) (ST3) (SL-3) transcripts detectable by or according to seg24, HSSTROL3 seg24 (SEQ ID NO:384) amplicon and HSSTROL3 seg24F (SEQ ID NO:382) and HSSTROL3 seg24R (SEQ ID NO:383) primers was measured by real time PCR. In parallel the expression of four housekeeping genes—PBGD (GenBank Accession No. BC019323 (SEQ ID NO:6); amplicon—PBGD-amplicon (SEQ ID NO:32), HPRT1 (GenBank Accession No. NM_{—}000194 (SEQ ID NO:5); amplicon—HPRT1-amplicon (SEQ ID NO:35)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 5, “Tissue samples in lung cancer testing panel”, above), to obtain a value of fold up-regulation for each sample relative to median of the normal PM samples.

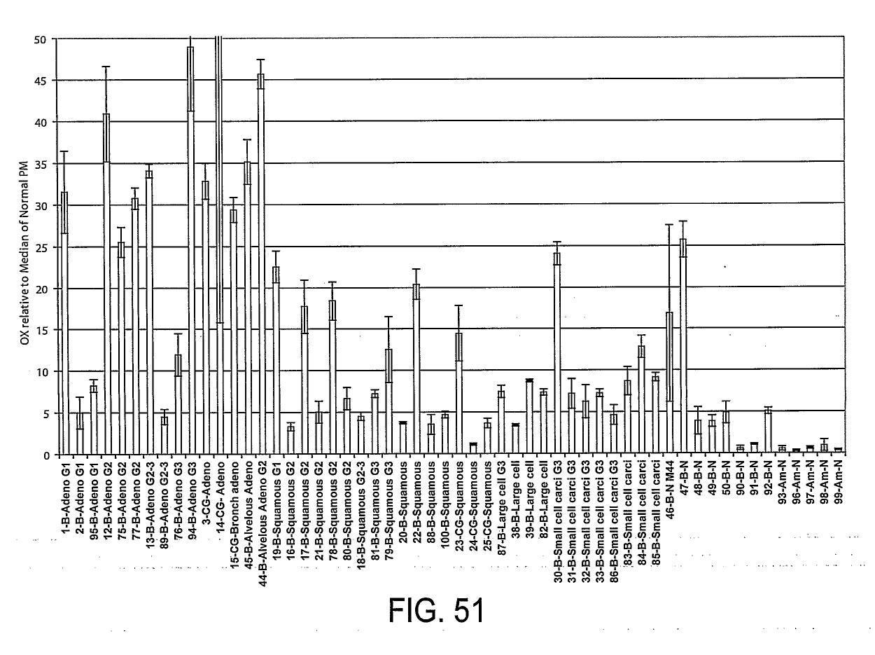

FIG. 51 is a histogram showing over expression of the above-indicated Stromelysin-3 precursor (SEQ ID NO:363) transcripts in cancerous lung samples relative to the normal samples. Values represent the average of duplicate experiments. Error bars indicate the minimal and maximal values obtained.)

As is evident from FIG. 51, the expression of Stromelysin-3 precursor (SEQ ID NO:363) transcripts detectable by the above amplicon in cancer samples was significantly higher than in the non-cancerous samples (Sample Nos. 47-50, 90-93, 96-99 Table 5, “Tissue samples in lung cancer testing panel”). Notably an over-expression of at least 5 fold was found in 13 out of 15 adenocarcinoma samples, 8 out of 16 squamous cell carcinoma samples, 3 out of 4 large cell carcinoma samples and in 7 out of 8 small cell carcinoma samples.

Threshold of 5 fold overexpression was found to differentiate between cancer and normal samples with P value of 4.04E-04 in adenocarcinoma, 9.89E-02 in squamous cell carcinoma, 6.04E-02 in Large cell carcinoma, 3.14E-03 in small cell carcinoma as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: HSSTROL3 seg24F (SEQ ID NO:382) forward primer; and HSSTROL3 seg24R (SEQ ID NO:383) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 seg24 (SEQ ID NO:384).

Forward Primer HSSTROL3 seg24 (SEQ ID NO:382):
ATTTCCATCCTCAACTGGCAGA
Reverse Primer HSSTROL3 seg24 (SEQ ID NO:383):
TGCCCTGGAACCCACG
Amplicon HSSTROL3 seg24 (SEQ ID NO:384):
ATTTCCATCCTCAACTGGCAGAGATGAGAGCCTGGAGCATTGCAGATGCC
AGGGACTTCACAAATGAAGGCACAGCATGGGAAACCTGCGTGGGTTCCAG
GGCA

Expression of Stromelysin-3 precursor (SEQ ID NO:363) (EC 3.4.24.-) (Matrix metalloproteinase-11) (MMP-11) (ST3) (SL-3)HSSTROL3 transcripts which are detectable by amplicon as depicted in sequence name HSSTROL3 seg24 (SEQ ID NO:384) in different normal tissues

Expression of Stromelysin-3 precursor (SEQ ID NO:363) (EC 3.4.24.-) (Matrix metalloproteinase-11) (MMP-11) (ST3) (SL-3) transcripts detectable by or according to HSSTROL3 seg24 (SEQ ID NO:384) amplicon and HSSTROL3 seg24F (SEQ ID NO:382) and HSSTROL3 seg24R (SEQ ID NO:383) was measured by real time PCR. In parallel the expression of four housekeeping genes UBC (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)) was measured similarly. For each RT sample, the expression of the above amplicon was normalized to the geometric mean of the quantities of the housekeeping genes. The normalized quantity of each RT sample was then divided by the median of the quantities of the breast samples (sample Nos. 33-35 Table 7, “Tissue samples in normal panel” above), to obtain a value of relative expression of each sample relative to median of the breast samples.

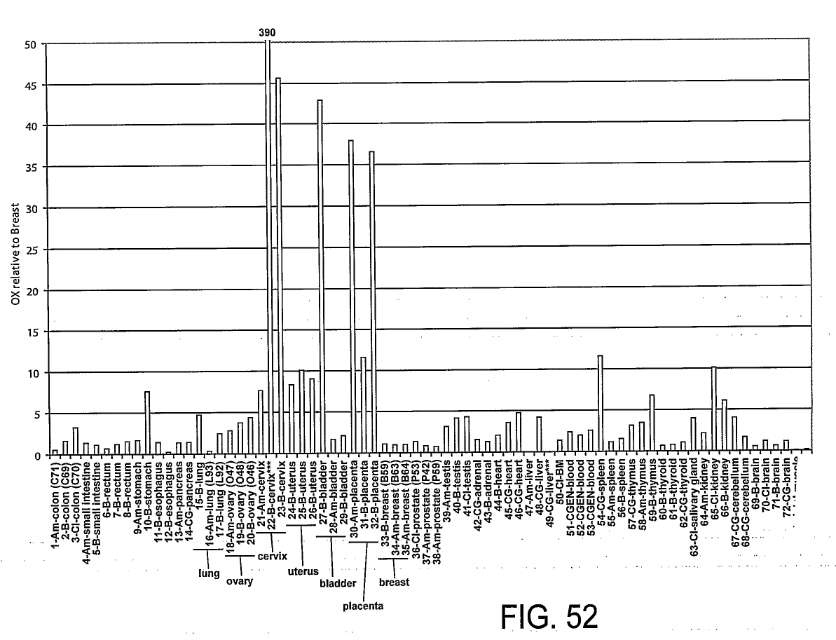

The results are presented in FIG. 52, demonstrating the expression of Stromelysin-3 precursor (SEQ ID NO:363) (EC 3.4.24.-) (Matrix metalloproteinase-11) (MMP-11) (ST3) (SL-3) HSSTROL3 transcripts, which are detectable by amplicon as depicted in sequence name HSSTROL3 seg24 (SEQ ID NO:384), in different normal tissues.

Forward Primer HSSTROL3 seg24 (SEQ ID NO:382):
ATTTCCATCCTCAACTGGCAGA
Reverse Primer HSSTROL3 seg24 (SEQ ID NO:383):
TGCCCTGGAACCCACG
Amplicon HSSTROL3 seg24 (SEQ ID NO:384):
ATTTCCATCCTCAACTGGCAGAGATGAGAGCCTGGAGCATTGCAGATGCC
AGGGACTTCACAAATGAAGGCACAGCATGGGAAACCTGCGTGGGTTCCAG
GGCA

Expression of Stromelysin-3 precursor (SEQ ID NO:363) (EC 3.4.24.-) (Matrix metalloproteinase-11) (MMP-11) (ST3) (SL-3HSSTROL3) transcripts which are detectable by amplicon as depicted in sequence name HSSTROL3 seg24 (SEQ ID NO:384) in normal and cancerous Prostate tissues

Expression of Stromelysin-3 precursor (SEQ ID NO:363) (EC 3.4.24.-) (Matrix metalloproteinase-11) (MMP-11) (ST3) (SL-3) transcripts detectable by or according to seg24, HSSTROL3 seg24 (SEQ ID NO:384) amplicon(s) and HSSTROL3 seg24F (SEQ ID NO:382) and HSSTROL3 seg24R (SEQ ID NO:383) primers was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and RPL19 (GenBank Accession No. NM_{—}000981 (SEQ ID NO:7); RPL19 amplicon (SEQ ID NO: 38)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 42, 48-53, 59-63, Table 2, “Tissue samples in prostate cancer testing panel”, above), to obtain a value of fold up-regulation for each sample relative to median of the normal PM samples.

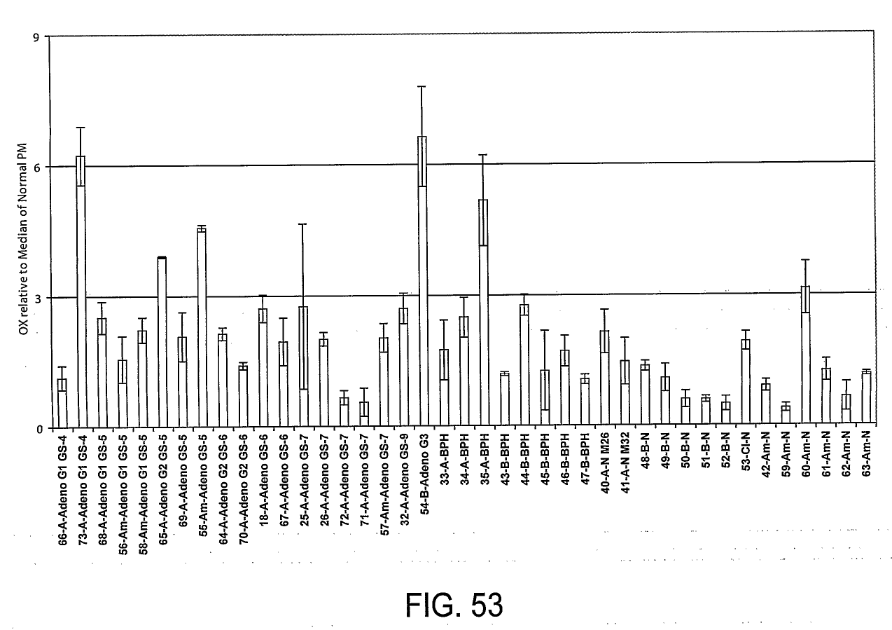

FIG. 53 is a histogram showing over expression of the above-indicated Stromelysin-3 precursor (SEQ ID NO:363) transcripts in cancerous Prostate samples relative to the normal samples. Values represent the average of duplicate experiments. Error bars indicate the minimal and maximal values obtained.

As is evident from FIG. 53, the expression of Stromelysin-3 precursor (SEQ ID NO:363) transcripts detectable by the above amplicon in cancer samples was higher than in several non-cancerous samples (Sample Nos. 42, 48-53, 59-63, Table 2, “Tissue samples in prostate cancer testing panel”). Notably an over-expression of at least 3 fold was found in 4 out of 19 adenocarcinoma samples.

Statistical analysis was applied to verify the significance of these results, as described below.

The P value for the difference in the expression levels of Stromelysin-3 precursor (SEQ ID NO:363) transcripts detectable by the above amplicon in Prostate cancer samples versus the normal tissue samples was determined by T test as 2.34E-03.

The above value demonstrates statistical significance of the results.

Primer pairs are also optionally and preferably encompassed within the present invention; for example, for the above experiment, the following primer pair was used as a non-limiting illustrative example only of a suitable primer pair: HSSTROL3 seg24F (SEQ ID NO:382) forward primer; and HSSTROL3 seg24R (SEQ ID NO:383) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL seg24 (SEQ ID NO: 384).

HSSTROL seg24 Forward primer (SEQ ID NO:382):
ATTTCCATCCTCAACTGGCAGA
HSSTROL seg24 Reverse primer (SEQ ID NO:383):
TGCCCTGGAACCCACG
HSSTROL seg24 Amplicon (SEQ ID NO:384):
ATTTCCATCCTCAACTGGCAGAGATGAGAGCCTGGAGCATTGCAGATGCC
AGGGACTTCACAAATGAAGGCACAGCATGGGAAACCTGCGTGGGTTCCAG
GGCA

Expression of Homo sapiens matrix metalloproteinase 11 (stromelysin 3) (MMP11) HSSTROL3 transcripts which are detectable by amplicon as depicted in sequence name HSSTROL3 seg25 (SEQ ID NO:387) in normal and cancerous colon tissues

Expression of Homo sapiens matrix metalloproteinase 11 (stromelysin 3) (MMP11) transcripts detectable by or according to seg25, HSSTROL3seg 25 (SEQ ID NO: 387) amplicon and primers HSSTROL3 seg25F (SEQ ID NO:385) and HSSTROL3 seg25R (SEQ ID NO:386) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), G6PD (GenBank Accession No. NM_{—}000402 (SEQ ID NO:8); G6PD-amplicon (SEQ ID NO: 44),), RPS27A (GenBank Accession No. NM_{—}002954 (SEQ ID NO:1); RPS27A amplicon (SEQ ID NO: 47)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 4, “Tissue samples in colon cancer testing panel”, above), to obtain a value of fold up-regulation for each sample relative to median of the normal PM samples.

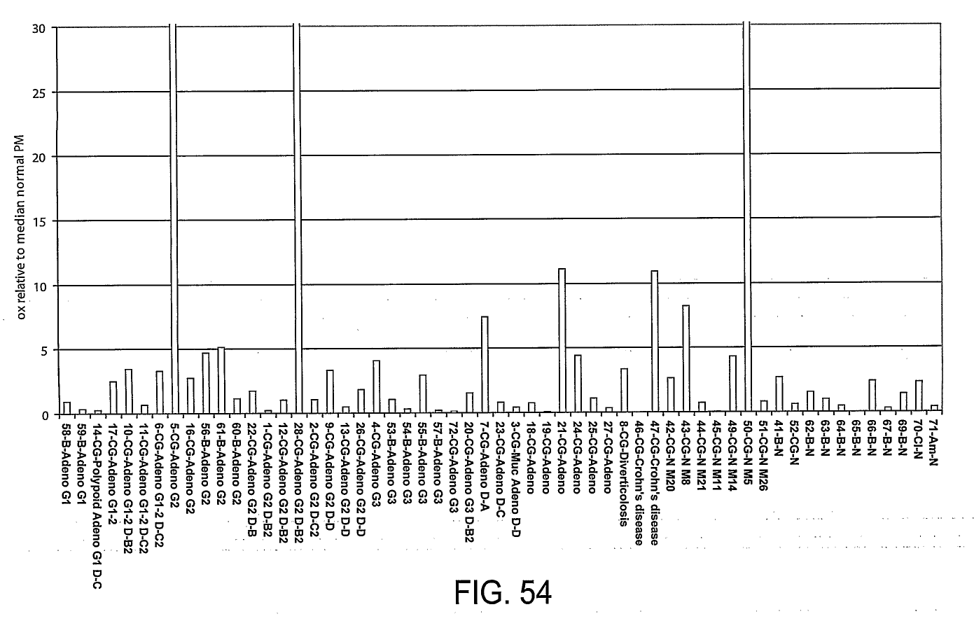

FIG. 54 is a histogram showing over expression of the above-indicated Homo sapiens matrix metalloproteinase 11 (stromelysin 3) (MMP11) transcripts in cancerous colon samples relative to the normal samples.

As is evident from FIG. 54, the expression of Homo sapiens matrix metalloproteinase 11 (stromelysin 3) (MMP11) transcripts detectable by the above amplicon was higher in a few cancer samples than in the non-cancerous samples (Sample Nos. 41, 52, 62-67, 69-71 Table 4, “Tissue samples in colon cancer testing panel”). Notably an over-expression of at least 5 fold was found in 5 out of 36 adenocarcinoma samples.

Primer pairs are also optionally and preferably encompassed within the present invention; for example, for the above experiment, the following primer pair was used as a non-limiting illustrative example only of a suitable primer pair: HSSTROL3 seg25F (SEQ ID NO:385) forward primer; and HSSTROL3 seg25R (SEQ ID NO:386) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 seg25 (SEQ ID NO:387).

Primers:

Forward primer HSSTROL3 seg25F (SEQ ID NO:385):
CACTGCCCCAGCTTATCCC
Reverse primer HSSTROL3 seg25R (SEQ ID NO:386):
CTCTCCCAGCCTCAGTTTCCT
Amplicon HSSTROL3 seg25 (SEQ ID NO:387):
CACTGCCCCAGCTTATCCCAGGCCTCCCGCTTCCCTCTGCGGGTGGGGTG
CTGAGCAGGCATTATTGGCCTGCATGTTTTACTGATGAGGAAACTGAGGC
TGGGAGAG

Expression of Stromelysin-3 precursor (SEQ ID NO:363) (EC 3.4.24.-) (Matrix metalloproteinase-11) (MMP-11) (ST3) (SL-3) HSSTROL3 transcripts which are detectable by amplicon as depicted in sequence name HSSTROL3 seg25 (SEQ ID NO:387) in normal and cancerous breast tissues

Expression of Stromelysin-3 precursor (SEQ ID NO:363) transcripts detectable by or according to seg25, HSSTROL3 seg25 (SEQ ID NO:387) amplicon and primers HSSTROL3 seg25F (SEQ ID NO:385) and HSSTROL3 seg25R (SEQ ID NO:386) was measured by real time PCR (RNA was as for the experiment above). In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), G6PD (GenBank Accession No. NM_{—}000402 (SEQ ID NO:8); G6PD amplicon (SEQ ID NO: 44)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67, Table 6, “Tissue samples in breast cancer testing panel”, above), to obtain a value of fold up-regulation for each sample relative to median of the normal PM samples.

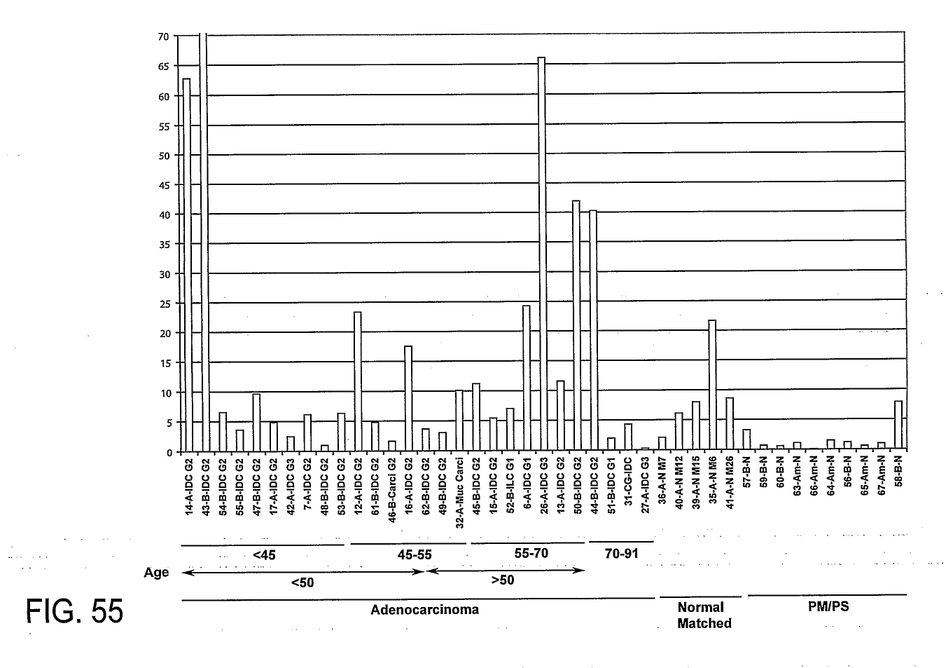

FIG. 55 is a histogram showing over expression of the above-indicated Stromelysin-3 precursor (SEQ ID NO:363) transcripts in cancerous breast samples relative to the normal samples.

As is evident from FIG. 55, the expression of Stromelysin-3 precursor (SEQ ID NO:363) transcripts detectable by the above amplicon in cancer samples was significantly higher than in the non-cancerous samples (Sample Nos. 56-60, 63-67 Table 6, “Tissue samples in breast cancer testing panel”, above). Notably an over-expression of at least 5 fold was found in 20 out of 28 adenocarcinoma samples.

Statistical analysis was applied to verify the significance of these results, as described below.

The P value for the difference in the expression levels of Stromelysin-3 precursor (SEQ ID NO:363) transcripts detectable by the above amplicon in breast cancer samples versus the normal tissue samples was determined by T test as 5.79E-02.

Threshold of 5 fold overexpression was found to differentiate between cancer and normal samples with P value of 6.75E-03 as checked by exact fisher test. The above values demonstrate statistical significance of the results.

By dissecting the patient's population into four age groups (indicated below the graph), we demonstrate that the transcripts detected by the above amplicon are highly expressed mainly in patient with age above 55 and/or patients with an age below 45.

Primer pairs are also optionally and preferably encompassed within the present invention; for example, for the above experiment, the following primer pair was used as a non-limiting illustrative example only of a suitable primer pair: HSSTROL seg25F (SEQ ID NO: 385) forward primer; and HSSTROL seg25R (SEQ ID NO: 386) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL seg25 (SEQ ID NO: 387).

Forward primer HSSTROL seg25F (SEQ ID NO:385):
CACTGCCCCAGCTTATCCC
Reverse primer HSSTROL seg25R (SEQ ID NO:386):
CTCTCCCAGCCTCAGTTTCCT
Amplicon HSSTROL seg25 (SEQ ID NO:387):
CACTGCCCCAGCTTATCCCAGGCCTCCCGCTTCCCTCTGCGGGTGGGGTG
CTGAGCAGGCATTATTGGCCTGCATGTTTTACTGATGAGGAAACTGAGGC
TGGGAGAG

Expression of Homo sapiens matrix metalloproteinase 11 (stromelysin 3) (MMP11) HSSTROL3 transcripts which are detectable by amplicon as depicted in sequence name HSSTROL3 seg25 (SEQ ID NO:387) in normal and cancerous lung tissues

Expression of Homo sapiens matrix metalloproteinase 11 (stromelysin 3) (MMP11) transcripts detectable by or according to seg25—HSSTROL3 seg25 (SEQ ID NO:387) amplicon and primers HSSTROL3 seg25F (SEQ ID NO:385) and HSSTROL3 seg25R (SEQ ID NO:386) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 5, above, “Tissue samples in lung cancer testing panel”), to obtain a value of fold up-regulation for each sample relative to median of the normal PM samples.

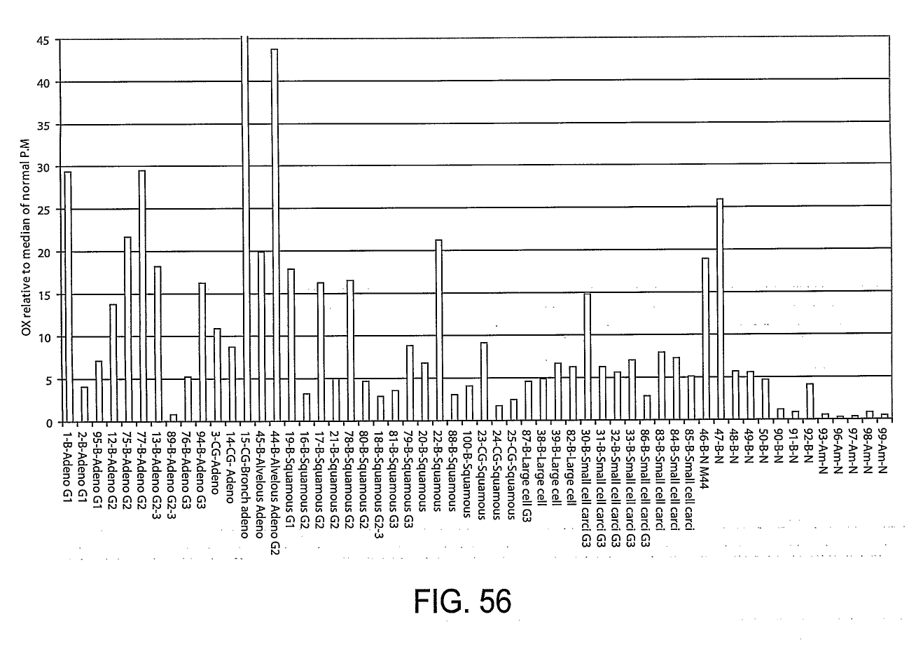

FIG. 56 is a histogram showing over expression of the above-indicated Homo sapiens matrix metalloproteinase 11 (stromelysin 3) (MMP11) transcripts in cancerous lung samples relative to the normal samples.

As is evident from FIG. 56, the expression of Homo sapiens matrix metalloproteinase 11 (stromelysin 3) (MMP11) transcripts detectable by the above amplicon in cancer samples was higher than in the non-cancerous samples (Sample Nos. 46-50, 90-93, 96-99 Table 5, above, “Tissue samples in lung cancer testing panel”). Notably an over-expression of at least 5 fold was found in 13 out of 15 adenocarcinoma samples, 8 out of 16 squamous cell carcinoma samples, 2 out of 4 large cell carcinoma samples and in 7 out of 8 small cells carcinoma samples.

Primer pairs are also optionally and preferably encompassed within the present invention; for example, for the above experiment, the following primer pair was used as a non-limiting illustrative example only of a suitable primer pair: HSSTROL3 seg25F (SEQ ID NO:385) forward primer; and HSSTROL3 seg25R (SEQ ID NO:386) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3.

Primers:

Forward primer HSSTROL3 seg25F (SEQ ID NO:385):
CACTGCCCCAGCTTATCCC
Reverse primer HSSTROL3 seg25R (SEQ ID NO:386):
CTCTCCCAGCCTCAGTTTCCT
Amplicon HSSTROL3 seg25 (SEQ ID NO:387):
CACTGCCCCAGCTTATCCCAGGCCTCCCGCTTCCCTCTGCGGGTGGGGTG
CTGAGCAGGCATTATTGGCCTGCATGTTTTACTGATGAGGAAACTGAGGC
TGGGAGAG

Expression of Homo sapiens matrix metalloproteinase 11 (stromelysin 3) (MMP11) HSSTROL3 transcripts which are detectable by amplicon as depicted in sequence name HSSTROL3 seg25 (SEQ ID NO:387) in normal and cancerous ovary tissues

Expression of Homo sapiens matrix metalloproteinase 11 (stromelysin 3) (MMP11) transcripts detectable by or according to seg25, HSSTROL3 seg25 (SEQ ID NO:387) amplicon and primers HSSTROL3 seg25F (SEQ ID NO:385) and HSSTROL3 seg25R (SEQ ID NO:386) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold differential expression for each sample relative to median of the normal PM samples.

In one experiment that was carried out no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: HSSTROL3 seg25F (SEQ ID NO:385) forward primer; and HSSTROL3 seg25R (SEQ ID NO:386) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL3 seg25 (SEQ ID NO:387).

Primers:

Forward primer HSSTROL3 seg25F (SEQ ID NO:385):
CACTGCCCCAGCTTATCCC
Reverse primer HSSTROL3 seg254R (SEQ ID NO:386):
CTCTCCCAGCCTCAGTTTCCT
Amplicon HSSTROL3 seg25 (SEQ ID NO:387):
CACTGCCCCAGCTTATCCCAGGCCTCCCGCTTCCCTCTGCGGGTGGGGTG
CTGAGCAGGCATTATTGGCCTGCATGTTTTACTGATGAGGAAACTGAGGC
TGGGAGAG

Expression of Stromelysin-3 precursor (SEQ ID NO:363) (EC 3.4.24.-) (Matrix metalloproteinase-11) (MMP-11) (ST3) (SL-3HSSTROL3) transcripts which are detectable by amplicon as depicted in sequence name HSSTROL3 seg25 (SEQ ID NO:387) in normal and cancerous Prostate tissues

Expression of Stromelysin-3 precursor (SEQ ID NO:363) (EC 3.4.24.-) (Matrix metalloproteinase-11) (MMP-11) (ST3) (SL-3) transcripts detectable by or according to seg25, HSSTROL3 seg25 (SEQ ID NO:387) amplicon and HSSTROL3 seg25F (SEQ ID NO:385) and HSSTROL3 seg25R (SEQ ID NO:386) primers was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and RPL19 (GenBank Accession No. NM_{—}000981 (SEQ ID NO:7); RPL19 amplicon (SEQ ID NO: 38)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 42, 48-53, 59-63, Table 2, “Tissue samples in prostate cancer testing panel”, above), to obtain a value of fold up-regulation for each sample relative to median of the normal PM samples.

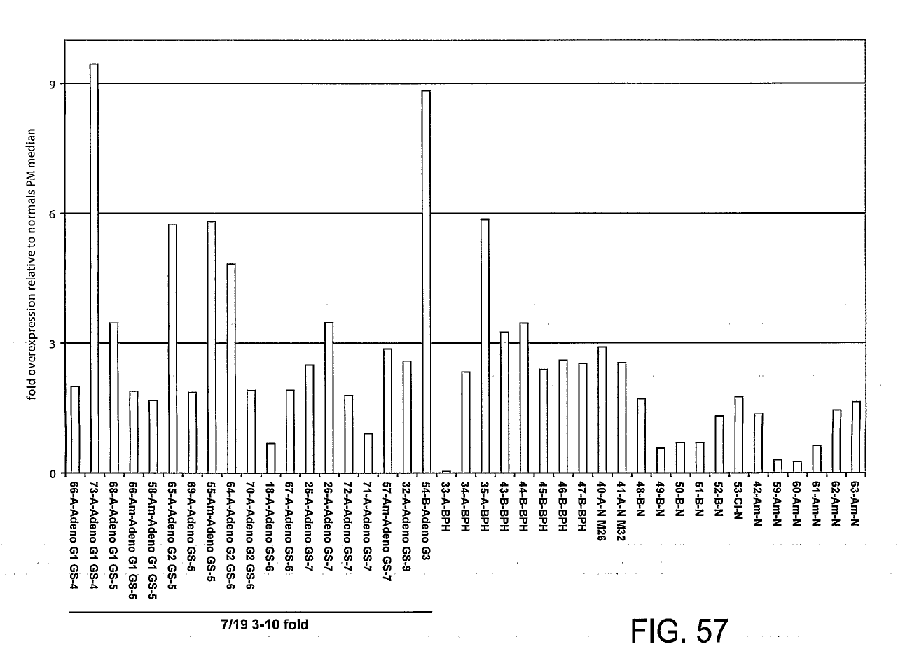

FIG. 57 is a histogram showing over expression of the above-indicated Stromelysin-3 precursor (SEQ ID NO:363) transcripts in cancerous Prostate samples relative to the normal samples. Values represent the average of duplicate experiments.

As is evident from FIG. 57, the expression of Stromelysin-3 precursor (SEQ ID NO:363) transcripts detectable by the above amplicon in cancer samples was higher than in several non-cancerous samples (Sample Nos. 42, 48-53, 59-63, Table 2, “Tissue samples in prostate cancer testing panel”). Notably an over-expression of at least 5 fold was found in 7 out of 19 adenocarcinoma samples.

Primer pairs are also optionally and preferably encompassed within the present invention; for example, for the above experiment, the following primer pair was used as a non-limiting illustrative example only of a suitable primer pair: HSSTROL3 seg25F (SEQ ID NO:385) forward primer; and HSSTROL3 seg25R (SEQ ID NO:386) reverse primer.

The present invention also preferably encompasses any amplicon obtained through the use of any suitable primer pair; for example, for the above experiment, the following amplicon was obtained as a non-limiting illustrative example only of a suitable amplicon: HSSTROL seg25 (SEQ ID NO: 387).

Primers:

Forward primer HSSTROL3 seg25F (SEQ ID NO:385):
CACTGCCCCAGCTTATCCC
Reverse primer HSSTROL3 seg25R (SEQ ID NO:386):
CTCTCCCAGCCTCAGTTTCCT
Amplicon HSSTROL3 seg25 (SEQ ID NO:387):
CACTGCCCCAGCTTATCCCAGGCCTCCCGCTTCCCTCTGCGGGTGGGGTG
CTGAGCAGGCATTATTGGCCTGCATGTTTTACTGATGAGGAAACTGAGGC
TGGGAGAG

### Description for Cluster HUMGRP5E

Cluster HUMGRP5E features 5 transcript(s) and 7 segment(s) of interest, the names for which are given in Tables 143 and 144, respectively. The selected protein variants are given in table 145.

TABLE 143
Transcripts of interest
Transcript Name

HUMGRP5E_T1 (SEQ ID NO: 388)
HUMGRP5E_T2 (SEQ ID NO: 389)
HUMGRP5E_T3 (SEQ ID NO: 390)
HUMGRP5E_T4 (SEQ ID NO: 391)
HUMGRP5E_T5 (SEQ ID NO: 392)

TABLE 144
Segments of interest

| Segment Name | Corresponding Transcript(s) |
|---|---|
| HUMGRP5E_node_0 | HUMGRP5E_T1 (SEQ ID NO: 388), |
| (SEQ ID NO: 393) | HUMGRP5E_T2 (SEQ ID NO: 389), |
| | HUMGRP5E_T4 (SEQ ID NO: 391) and |
| | HUMGRP5E_T5 (SEQ ID NO: 392) |
| HUMGRP5E_node_2 | HUMGRP5E_T1 (SEQ ID NO: 388), |
| (SEQ ID NO: 394) | HUMGRP5E_T2 (SEQ ID NO: 389), |
| | HUMGRP5E_T4 (SEQ ID NO: 391) and |
| | HUMGRP5E_T5 (SEQ ID NO: 392) |
| HUMGRP5E_node_5 | HUMGRP5E_T3 (SEQ ID NO: 390) |
| (SEQ ID NO: 395) | |
| HUMGRP5E_node_8 | HUMGRP5E_T1 (SEQ ID NO: 388), |
| (SEQ ID NO: 396) | HUMGRP5E_T2 (SEQ ID NO: 389), |
| | HUMGRP5E_T3 (SEQ ID NO: 390), |
| | HUMGRP5E_T4 (SEQ ID NO: 391) and |
| | HUMGRP5E_T5 (SEQ ID NO: 392) |
| HUMGRP5E_node_3 | HUMGRP5E_T4 (SEQ ID NO: 391) and |
| (SEQ ID NO: 397) | HUMGRP5E_T5 (SEQ ID NO: 392) |
| HUMGRP5E_node_6 | HUMGRP5E_T1 (SEQ ID NO: 388) and |
| (SEQ ID NO: 398) | HUMGRP5E_T3 (SEQ ID NO: 390) |
| HUMGRP5E_node_7 | HUMGRP5E_T1 (SEQ ID NO: 388), |
| (SEQ ID NO: 399) | HUMGRP5E_T2 (SEQ ID NO: 389), |
| | HUMGRP5E_T3 (SEQ ID NO: 390) and |
| | HUMGRP5E_T5 (SEQ ID NO: 392) |

TABLE 145
Proteins of interest

| Protein Name | Corresponding Transcript(s) |
|---|---|
| HUMGRP5E_P2 | HUMGRP5E_T1 (SEQ ID NO: 388) |
| (SEQ ID NO: 401) | |
| HUMGRP5E_P3 (SEQ ID | HUMGRP5E_T2 (SEQ ID NO: 389) |
| NO: 402) | |
| HUMGRP5E_P4 (SEQ ID | HUMGRP5E_T4 (SEQ ID NO: 391) |
| NO: 403) | |
| HUMGRP5E_P5 (SEQ ID NO: | HUMGRP5E_T5 (SEQ ID NO: 392) |
| 404) | |

These sequences are variants of the known protein Gastrin-releasing peptide precursor (SEQ ID NO:400) (SwissProt accession identifier GRP_HUMAN (SEQ ID NO:400); known also according to the synonyms GRP; GRP-10), referred to herein as the previously known protein.

Protein Gastrin-releasing peptide precursor (SEQ ID NO:400) is known or believed to have the following function(s): GRP stimulates gastrin release as well as other gastrointestinal hormones. Known polymorphisms for this sequence are as shown in Table 146.

TABLE 146
Amino acid mutations for Known Protein

| SNP position(s) on amino | |
|---|---|
| acid sequence | Comment |
| 4 | S -> R |

Protein Gastrin-releasing peptide precursor (SEQ ID NO:400) localization is believed to be Secreted.

The previously known protein also has the following indication(s) and/or potential therapeutic use(s): Diabetes, Type II. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Bombesin antagonist; Insulinotropin agonist. A therapeutic role for a protein represented by the cluster has been predicted. The cluster was assigned this field because there was information in the drug database or the public databases (e.g., described herein above) that this protein, or part thereof, is used or can be used for a potential therapeutic indication: Anorectic/Antiobesity; Releasing hormone; Anticancer; Respiratory; Antidiabetic.

The following GO Annotation(s) apply to the previously known protein. The following annotation(s) were found: signal transduction; neuropeptide signaling pathway, which are annotation(s) related to Biological Process; growth factor, which are annotation(s) related to Molecular Function; and soluble fraction, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of HUMGRP5E) may optionally have one or more of the following utilities, as described with regard to Table 147 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 147
Utilities for Variants of HUMGRP5E

| Utility | Reason | Reference |
|---|---|---|
| Detection of esophageal | Significant over-expression of GRP | Carcinogenesis. 2004 |
| squamous cell carcinomas by | was observed in 10 out of 12 ESCC | Jun; 25(6): 865-71. |
| mRNA or protein assay. | samples (83.3%) and all four ESCC | J Natl Cancer Inst. 2002 |
| | cell lines. | Mar 6; 94(5): 375-83. |
| | | Oncogene. 2003 Sep |
| | | 18; 22(40): 6183-93. |
| Detection and diagnosis of | The serum Pro-GRP levels were | Anticancer Res. 2003 Mar-Apr; |
| Small Cell lung cancer and | elevated in SCLC patients. | 23(2A): 895-8. |
| carcinoid lung tumors by | In the 9 patients with SCLC who | Anticancer Res. 1999 Jul-Aug; |
| mRNA or protein essay for GRP | relapsed, the serum Pro-GRP levels | 19(4A): 2673-8. |
| or proGRP; also a marker for | were again elevated at the time of | Oncology. 1999; 57(2): 143-8. |
| treatment monitoring and | relapse. | Lung Cancer. 2000 |
| survival in small-cell lung | | Mar; 27(3): 159-67. |
| cancer. | | Jpn J Cancer Res. 1995 |
| | | Jul; 86(7): 698-705. |
| | | Cancer Res. 1994 Apr |
| | | 15; 54(8): 2136-40. |
| | | Clin Lab. 2003; 49(1-2): 35-42. |
| | | Proc. Nat. Acad. Sci. 81: |
| | | 5699-5703, 1984. |
| Detection of medullary thyroid | Serum Pro-GRP levels were elevated | Thyroid. 2001 |
| carcinoma by use of mRNA or | in 80% (12/15) patients. | Nov; 11(11): 1055-61. |
| protein assay for GRP or pro- | | Oncology. 2000 |
| GRP | | Aug; 59(2): 122-5. |
| | | Metabolism. 1984 |
| | | Aug; 33(8): 724-7. |
| Detection and diagnosis of | All of the patients with normal renal | Nihon Kokyuki Gakkai |
| chronic renal failure by use of | function, whether they had diabetes | Zasshi. 2002 |
| protein assay for GRP or pro- | mellitus (n = 16), rheumatoid arthritis | May; 40(5): 369-72 |
| GRP | (n = 10), systemic lupus erythematosus | Nephrol Dial Transplant. |
| | (n = 12) or chronic glomerulonephritis | 1996 Jul; 11(7): 1267-70. |
| | (n = 14), had serum pro-gastrin- | Respirology. 1998 |
| | releasing peptide concentrations less | Sep; 3(3): 207-10. |
| | than 46 ng/l, the upper limit in normal | |
| | subjects. In contrast, 14 or 16 patients | |
| | (88%) with small-cell lung carcinoma, | |
| | who had normal renal function, and 25 | |
| | of 26 (96%) patients with chronic renal | |
| | failure on haemodialysis had serum | |
| | pro-gastrin-releasing peptide | |
| | concentrations greater than 46 ng/l. | |
| Detection, diagnosis and | GRP was expressed in 18 radical | World J Urol. 2003 |
| progression prediction | prostatectomy specimens (60%) and in | Aug; 21(3): 183-7. Epub |
| (prognosis) in prostate cancer by | 15 biopsies (50%). There was an | 2003 Jul 5. |
| use of protein assay for GRP or | association between positive | Int J Cancer. 1998 Feb |
| pro-GRP. | immunoexpression of GRP, relapse | 20; 79(1): 82-90. |
| | (P = 0.029) and advanced tumor stages | Cancer. 2004 Aug |
| | (i.e. pT3, pT4) (P = 0.049). | 1; 101(3): 527-32. |
| Detection of melanoma, | Existence of GRP in all | Melanoma Res. 2000 |
| particularly nodular melanomas, | clinicopathological types of melanoma; | Aug; 10(4): 395-400. |
| by use of protein assay for GRP | a predilection for quantitatively | J Dermatol Sci. 1998 |
| or pro-GRP. | increased GRP immunostaining was | Jun; 17(2): 93-100. |
| | noticed in nodular melanomas (P = | |
| | 0.007). | |
| Detection of early stages of | BN/GRP-like peptide is present in the | Clin Exp Rheumatol. 2001 |
| rheumatoid arthritis by use of | synovial fluid of joints affected by | Nov-Dec; 19(6): 715-20. |
| protein levels in serum and | arthritis and that the pattern of | |
| synovial fluid. | BN/GRP increase differs from that of | |
| | SP. It appears as if the presence of | |
| | BN/GRP is particularly related to the | |
| | early processes of joint involvement. | |
| Detection of retroperitoneal | All the conditioned media from the | Am J Surg. 1995 |
| pediatric malignant tumors | benign tumors contained <25 pg/mL | May; 169(5): 550-2. |
| (such as ganglioneuromas, | net GRP, whereas all the malignant | Ann Surg. 2002 |
| neuroblastoma, primitive | tumor-conditioned media contained > | May; 235(5): 621-9; |
| neuroectodermal tumor, Ewing | or = 45 pg/mL (P = 0.003). | discussion 629-30. |
| tumor family, ect.). | | Curr Surg. 2001 |
| | | Jan; 58(1): 86-89. |
| | | Cancer Res. 1998 Jun |
| | | 1; 58(11): 2469-76. |
| Monitoring Helicobacter pylori | GRP levels are higher in HP positive | 1. Gastroenterology. 1995 |
| eradication in duodenal ulcers | patients, and its levels are reduced after | Sep; 109(3): 681-91. |
| patients. | treatment and HP eradication. | 1. Aliment Pharmacol |
| | | Ther. 1995 Aug; 9(4): 341-7. |
| | | Review |
| | | 2. Gut. 1996 |
| | | May; 38(5): 663-7. |
| Breast cancer | Bombesin or gastrin-releasing peptide | Eur J Cancer. 1998 |
| | (GRP) may act as autocrine growth | Apr; 34(5): 710-7 |
| | factors and play a role in the initiation | |
| | and progression of breast cancer | |

According to other optional embodiments of the present invention, variants of this cluster according to the present invention (amino acid and/or nucleic acid sequences of HUMGRP5E) may optionally have one or more of the following utilities, some of which are related to utilities described above. It should be noted that these utilities are optionally and preferably suitable for human and non-human animals as subjects, except where otherwise noted.

A non-limiting example of such a utility is detecting and/or diagnosing cancer, and/or the immune response of a subject to cancer, by using at least one HUMGRP5E variant according to the present invention. Such use of the known protein is described with regard to US Patent Application No. 2003/0232399, hereby incorporated by reference as if fully set forth herein.

Another non-limiting example of such a utility is diagnosing and/or monitoring testicular cancer in a subject, by using at least one HUMGRP5E variant according to the present invention. Such use of the known protein is described with regard to PCT Application No. WO 03/044224, hereby incorporated by reference as if fully set forth herein.

Yet another non-limiting example of such a utility is diagnosing and/or monitoring lung cancer in a subject, optionally and preferably small cell lung cancer, by using at least one HUMGRP5E variant according to the present invention. Such use of the known protein is described with regard to U.S. Pat. No. 5,770,385, hereby incorporated by reference as if fully set forth herein.

Table 148 below describes diagnostic utilities for the cluster HUMGRP5E that were found through microarrays, including the statistical significance thereof and a reference. One or more HUMGRP5E variants according to the present invention may optionally have one or more of these utilities.

TABLE 148

| | Statistical | |
|---|---|---|
| Diagnostic utility | significance | reference |
| Gene over expression in small cell lung | Ranging | Bhattacharjee A, Meyerson M PNAS |
| cancer (vs. normal and other types of | between 0.002 | (2001) Classification of human lung |
| lung cancer). | to 5e⁻⁴ | carcinomas by mRNA expression profiling |
| | | reveals distinct adenocarcinoma |
| | | subclasses. |
| | | Garber ME, Petersen I PNAS (2001) |
| | | Diversity of gene expression in |
| | | adenocarcinoma of the lung |
| Gene over expression in carcinoid lung | 4e⁻⁴ | Bhattacharjee A, Meyerson M PNAS |
| cancer (vs. normal and other types of | | (2001) Classification of human lung |
| lung cancer). | | carcinomas by mRNA expression profiling |
| | | reveals distinct adenocarcinoma |
| | | subclasses. |
| Breast cancer prognosis, gene over | 0.036, 9.3e⁻¹¹, | Sorlie T, Borresen-Dale AL PNAS (2001) |
| expressed in patients with better survival | 0.001 | Gene expression patterns of breast |
| prediction (ER positive, negative lymph | respectively | carcinomas distinguish tumor subclasses |
| infiltrate and are in grade 1) | | with clinical implications. |
| | | Van't Veer LJ, Friend SH Nature (2002) |
| | | Gene expression profiling predicts clinical |
| | | outcome of breast cancer. |
| | | Perou CM, Botstein D Nature (2000) |
| | | Molecular portraits of human breast |
| | | tumours. |
| Gene over expressed in severe | | GNF database |
| emphysema (vs. normal lung or mild | | (http://www.ncbi.nlm.nih.gov/projects/geo/): |
| emphysema). | | GDS737, probe ID: 206326_at. |

Also, microarrays have shown that one or more HUMGRP5E variants according to the present invention may optionally be either overexpressed in lung cancer, preferably carcinoid non small cell lung cancer, or alternatively may be underexpressed in lung cancer, preferably adenocarcinoma.

Other non-limiting exemplary utilities for HUMGRP5E variants according to the present invention are described in greater detail below and also with regard to the previous section on clinical utility.

For this cluster, at least one oligonucleotide was found to demonstrate overexpression of the cluster, although not of at least one transcript/segment as listed below. Microarray (chip) data is also available for this cluster as follows. Various oligonucleotides were tested for being differentially expressed in various disease conditions, particularly cancer, as previously described. The following oligonucleotides were found to hit this cluster but not other segments/transcripts below, shown in Table 149.

TABLE 149
Oligonucleotides related to this cluster

| | | Chip |
|---|---|---|
| Oligonucleotide name | Overexpressed in cancers | reference |
| HUMGRP5E_0_0_16630 (SEQ | lung malignant tumors | LUN |
| ID NO: 10) | | |

As noted above, cluster HUMGRP5E features 5 transcript(s), which were listed in Table 143 above. These transcript(s) encode for protein(s) which are variant(s) of protein Gastrin-releasing peptide precursor (SEQ ID NO:400). A description of each variant protein according to the present invention is now provided.

Variant protein HUMGRP5E_P2 (SEQ ID NO:401) according to the present invention has an amino acid sequence; it is encoded by transcript(s) HUMGRP5E_T1 (SEQ ID NO:388). An alignment is given to the known protein (Gastrin-releasing peptide precursor (SEQ ID NO:400)) at the end of the application. One or more alignments to one or more previously published protein sequences are given in the alignment table located on the attached CDROM. A brief description of the relationship of the variant protein according to the present invention to each such aligned protein is as follows:

Comparison report between HUMGRP5E_P2 (SEQ ID NO:401) and GRP_HUMAN (SEQ ID NO:400):

1. An isolated chimeric polypeptide encoding for HUMGRP5E_P2 (SEQ ID NO:401), comprising a first amino acid sequence being at least 90% homologous to MRGSELPLVLLALVLCLAPRGRAVPLPAGGGTVLTKMYPRGNHWAVGHLMGKKSTGESSSVSER GSLKQQLREYIRWEEAARNLLGLIEAKENRNHQPPQPKALGNQQPSWDSEDSSNFKD corresponding to amino acids 1-121 of GRP_HUMAN (SEQ ID NO:400), which also corresponds to amino acids 1-121 of HUMGRP5E_P2 (SEQ ID NO:401), and a second amino acid sequence being at least 70%, optionally at least 80%, preferably at least 85%, more preferably at least 90% and most preferably at least 95% homologous to a polypeptide having the sequence LVDSLLQVLNVKEGTPS (SEQ ID NO: 657) corresponding to amino acids 122-138 of HUMGRP5E_P2 (SEQ ID NO:401), wherein said first amino acid sequence and second amino acid sequence are contiguous and in a sequential order.

2. An isolated polypeptide encoding for a tail of HUMGRP5E_P2 (SEQ ID NO:401), comprising a polypeptide being at least 70%, optionally at least about 80%, preferably at least about 85%, more preferably at least about 90% and most preferably at least about 95% homologous to the sequence LVDSLLQVLNVKEGTPS (SEQ ID NO: 657) in HUMGRP5E_P2 (SEQ ID NO:401).

The location of the variant protein was determined according to results from a number of different software programs and analyses, including analyses from SignalP and other specialized programs. The variant protein is believed to be located as follows with regard to the cell: secreted.

The phosphorylation sites of variant protein HUMGRP5E_P2 (SEQ ID NO:401), as compared to the known protein Gastrin-releasing peptide precursor (SEQ ID NO:400), are described in Table 150 (given according to their position(s) on the amino acid sequence in the first column; the second column indicates whether the phosphorylation site is present in the variant protein; and the last column indicates whether the position is different on the variant protein).

TABLE 150
Phosphorylation site(s)

| Position(s) on known | | Position in |
|---|---|---|
| amino acid sequence | Present in variant protein? | variant protein? |
| 50 | yes | 50 |

The variant protein has the following domains, as determined by using InterPro. The domains are described in Table 151:

TABLE 151
InterPro domain(s)

| | | | Position(s) |
|---|---|---|---|
| InterPro ID | Domain description | Analysis type | on protein |
| IPR000874 | Bombesin-like peptide | HMMPfam | 41-54 |
| IPR000874 | Bombesin-like peptide | ScanRegExp | 44-50 |

Variant protein HUMGRP5E_P2 (SEQ ID NO:401) is encoded by the following transcript(s): HUMGRP5E_T1 (SEQ ID NO:388). The coding portion of transcript HUMGRP5E_T1 (SEQ ID NO:388) starts at position 622 and ends at position 1035. The transcript also has the following SNPs as listed in Table 152 (given according to their position on the nucleotide sequence, with the alternative nucleic acid listed; the last column indicates whether the SNP is known or not; the presence of known SNPs in variant protein HUMGRP5E_P2 (SEQ ID NO:401) sequence provides support for the deduced sequence of this variant protein according to the present invention).

TABLE 152
Nucleic acid SNPs

| SNP position on | | |
|---|---|---|
| nucleotide sequence | Alternative nucleic acid | Previously known SNP? |
| 541 | -> T | No |
| 542 | G -> T | No |
| 631 | A -> C | Yes |
| 672 | G -> A | Yes |
| 1342 | C -> | No |
| 1342 | C -> A | No |
| 1343 | A -> | No |
| 1343 | A -> G | No |

Variant protein HUMGRP5E_P3 (SEQ ID NO:402) according to the present invention has an amino acid sequence; it is encoded by transcript(s) HUMGRP5E_T2 (SEQ ID NO:389). An alignment is given to the known protein (Gastrin-releasing peptide precursor (SEQ ID NO:400)). One or more alignments to one or more previously published protein sequences are given at the in the alignment table located on the attached CDROM. A brief description of the relationship of the variant protein according to the present invention to each such aligned protein is as follows:

Comparison report between HUMGRP5E_P3 (SEQ ID NO:402) and GRP_HUMAN (SEQ ID NO:400):

1. An isolated chimeric polypeptide encoding for HUMGRP5E_P3 (SEQ ID NO:402), comprising a first amino acid sequence being at least 90% homologous to MRGSELPLVLLALVLCLAPRGRAVPLPAGGGTVLTKMYPRGNHWAVGHLMGKKSTGESSSVSER GSLKQQLREYIRWEEAARNLLGLIEAKENRNHQPPQPKALGNQQPSWDSEDSSNFKD corresponding to amino acids 1-121 of GRP_HUMAN (SEQ ID NO:400), which also corresponds to amino acids 1-121 of HUMGRP5E_P3 (SEQ ID NO:402), and a second amino acid sequence being at least 70%, optionally at least 80%, preferably at least 85%, more preferably at least 90% and most preferably at least 95% homologous to a polypeptide having the sequence TLCSRFST (SEQ ID NO: 658) corresponding to amino acids 122-129 of HUMGRP5E_P3 (SEQ ID NO:402), wherein said first amino acid sequence and second amino acid sequence are contiguous and in a sequential order.

2. An isolated polypeptide encoding for a tail of HUMGRP5E_P3 (SEQ ID NO:402), comprising a polypeptide being at least 70%, optionally at least about 80%, preferably at least about 85%, more preferably at least about 90% and most preferably at least about 95% homologous to the sequence TLCSRFST (SEQ ID NO: 658) in HUMGRP5E_P3 (SEQ ID NO:402).

The location of the variant protein was determined according to results from a number of different software programs and analyses, including analyses from SignalP and other specialized programs. The variant protein is believed to be located as follows with regard to the cell: secreted.

The phosphorylation sites of variant protein HUMGRP5E_P3 (SEQ ID NO:402), as compared to the known protein Gastrin-releasing peptide precursor (SEQ ID NO:400), are described in Table 153 (given according to their position(s) on the amino acid sequence in the first column; the second column indicates whether the phosphorylation site is present in the variant protein; and the last column indicates whether the position is different on the variant protein).

TABLE 153
Phosphorylation site(s)

| Position(s) on known | | Position |
|---|---|---|
| amino acid sequence | Present in variant protein? | in variant protein? |
| 50 | yes | 50 |

The variant protein has the following domains, as determined by using InterPro. The domains are described in Table 154:

TABLE 154
InterPro domain(s)

| | | | Position(s) on |
|---|---|---|---|
| InterPro ID | Domain description | Analysis type | protein |
| IPR000874 | Bombesin-like peptide | HMMPfam | 41-54 |
| IPR000874 | Bombesin-like peptide | ScanRegExp | 44-50 |

Variant protein HUMGRP5E_P3 (SEQ ID NO:402) is encoded by the following transcript(s): HUMGRP5E_T2 (SEQ ID NO:389). The coding portion of transcript HUMGRP5E_T2 (SEQ ID NO:389) starts at position 622 and ends at position 1008. The transcript also has the following SNPs as listed in Table 155 (given according to their position on the nucleotide sequence, with the alternative nucleic acid listed; the last column indicates whether the SNP is known or not; the presence of known SNPs in variant protein HUMGRP5E_P3 (SEQ ID NO:402) sequence provides support for the deduced sequence of this variant protein according to the present invention).

TABLE 155
Nucleic acid SNPs

| SNP position on | | |
|---|---|---|
| nucleotide sequence | Alternative nucleic acid | Previously known SNP? |
| 541 | -> T | No |
| 542 | G -> T | No |
| 631 | A -> C | Yes |
| 672 | G -> A | Yes |
| 1335 | C -> | No |
| 1335 | C -> A | No |
| 1336 | A -> | No |
| 1336 | A -> G | No |

Variant protein HUMGRP5E_P4 (SEQ ID NO:403) according to the present invention has an amino acid sequence; it is encoded by transcript(s) HUMGRP5E_T4 (SEQ ID NO:391). An alignment is given to the known protein (Gastrin-releasing peptide precursor (SEQ ID NO:400)) at the end of the application. One or more alignments to one or more previously published protein sequences are given in the alignment table located on the attached CDROM. A brief description of the relationship of the variant protein according to the present invention to each such aligned protein is as follows:

Comparison report between HUMGRP5E_P4 (SEQ ID NO:403) and GRP_HUMAN (SEQ ID NO:400):

1. An isolated chimeric polypeptide encoding for HUMGRP5E_P4 (SEQ ID NO:403), comprising a first amino acid sequence being at least 90% homologous to MRGSELPLVLLALVLCLAPRGRAVPLPAGGGTVLTKMYPRGNHWAVGHLMGKKSTGESSSVSER GSLKQQLREYIRWEEAARNLLGLIEAKENRNHQPPQPKALGNQQPSWDSEDSSNFKDVGSKGK corresponding to amino acids 1-127 of GRP_HUMAN (SEQ ID NO:400), which also corresponds to amino acids 1-127 of HUMGRP5E_P4 (SEQ ID NO:403), and a second amino acid sequence being at least 90% homologous to GSQREGRNPQLNQQ corresponding to amino acids 135-148 of GRP_HUMAN (SEQ ID NO:400), which also corresponds to amino acids 128-141 of HUMGRP5E_P4 (SEQ ID NO:403), wherein said first amino acid sequence and second amino acid sequence are contiguous and in a sequential order.

2. An isolated chimeric polypeptide encoding for an edge portion of HUMGRP5E_P4 (SEQ ID NO:403), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KG, having a structure as follows: a sequence starting from any of amino acid numbers 127−x to 127; and ending at any of amino acid numbers 128+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted.

The phosphorylation sites of variant protein HUMGRP5E_P4 (SEQ ID NO:403), as compared to the known protein Gastrin-releasing peptide precursor (SEQ ID NO:400), are described in Table 156 (given according to their position(s) on the amino acid sequence in the first column; the second column indicates whether the phosphorylation site is present in the variant protein; and the last column indicates whether the position is different on the variant protein).

TABLE 156
Phosphorylation site(s)

| Position(s) on known amino | | Position in |
|---|---|---|
| acid sequence | Present in variant protein? | variant protein? |
| 50 | yes | 50 |

The variant protein has the following domains, as determined by using InterPro. The domains are described in Table 157:

TABLE 157
InterPro domain(s)

| | | | Position(s) on |
|---|---|---|---|
| InterPro ID | Domain description | Analysis type | protein |
| IPR000874 | Bombesin-like peptide | HMMPfam | 41-54 |
| IPR000874 | Bombesin-like peptide | ScanRegExp | 44-50 |

Variant protein HUMGRP5E_P4 (SEQ ID NO:403) is encoded by the following transcript(s): HUMGRP5E_T4 (SEQ ID NO:391). The coding portion of transcript HUMGRP5E_T4 (SEQ ID NO:391) starts at position 622 and ends at position 1044. The transcript also has the following SNPs as listed in Table 158 (given according to their position on the nucleotide sequence, with the alternative nucleic acid listed; the last column indicates whether the SNP is known or not; the presence of known SNPs in variant protein HUMGRP5E_P4 (SEQ ID NO:403) sequence provides support for the deduced sequence of this variant protein according to the present invention).

TABLE 158
Nucleic acid SNPs

| SNP position on | | |
|---|---|---|
| nucleotide sequence | Alternative nucleic acid | Previously known SNP? |
| 541 | -> T | No |
| 542 | G -> T | No |
| 631 | A -> C | Yes |
| 672 | G -> A | Yes |
| 1340 | C -> | No |
| 1340 | C -> A | No |
| 1341 | A -> | No |
| 1341 | A -> G | No |

Variant protein HUMGRP5E_P5 (SEQ ID NO:404) according to the present invention has an amino acid sequence; it is encoded by transcript(s) HUMGRP5E_T5 (SEQ ID NO:392). An alignment is given to the known protein (Gastrin-releasing peptide precursor (SEQ ID NO:400)). One or more alignments to one or more previously published protein sequences are given in the alignment table located on the attached CDROM. A brief description of the relationship of the variant protein according to the present invention to each such aligned protein is as follows:

Comparison report between HUMGRP5E_P5 (SEQ ID NO:404) and GRP_HUMAN (SEQ ID NO:400):

1. An isolated chimeric polypeptide encoding for HUMGRP5E_P5 (SEQ ID NO:404), comprising a first amino acid sequence being at least 90% homologous to MRGSELPLVLLALVLCLAPRGRAVPLPAGGGTVLTKMYPRGNHWAVGHLMGKKSTGESSSVSER GSLKQQLREYIRWEEAARNLLGLIEAKENRNHQPPQPKALGNQQPSWDSEDSSNFKDVGSKGK corresponding to amino acids 1-127 of GRP_HUMAN (SEQ ID NO:400), which also corresponds to amino acids 1-127 of HUMGRP5E_P5 (SEQ ID NO:404), and a second amino acid sequence being at least 70%, optionally at least 80%, preferably at least 85%, more preferably at least 90% and most preferably at least 95% homologous to a polypeptide having the sequence DSLLQVLNVKEGTPS (SEQ ID NO: 659) corresponding to amino acids 128-142 of HUMGRP5E_P5 (SEQ ID NO:404), wherein said first amino acid sequence and second amino acid sequence are contiguous and in a sequential order.

2. An isolated polypeptide encoding for a tail of HUMGRP5E_P5 (SEQ ID NO:404), comprising a polypeptide being at least 70%, optionally at least about 80%, preferably at least about 85%, more preferably at least about 90% and most preferably at least about 95% homologous to the sequence DSLLQVLNVKEGTPS (SEQ ID NO: 659) in HUMGRP5E_P5 (SEQ ID NO:404).

The location of the variant protein was determined according to results from a number of different software programs and analyses, including analyses from SignalP and other specialized programs. The variant protein is believed to be located as follows with regard to the cell: secreted.

The phosphorylation sites of variant protein HUMGRP5E_P5 (SEQ ID NO:404), as compared to the known protein Gastrin-releasing peptide precursor (SEQ ID NO:400), are described in Table 159 (given according to their position(s) on the amino acid sequence in the first column; the second column indicates whether the phosphorylation site is present in the variant protein; and the last column indicates whether the position is different on the variant protein).

TABLE 159
Phosphorylation site(s)

| Position(s) on known amino acid | Present in variant | Position in |
|---|---|---|
| sequence | protein? | variant protein? |
| 50 | yes | 50 |

The variant protein has the following domains, as determined by using InterPro. The domains are described in Table 160:

TABLE 160
InterPro domain(s)

| | | | Position(s) |
|---|---|---|---|
| InterPro ID | Domain description | Analysis type | on protein |
| IPR000874 | Bombesin-like peptide | HMMPfam | 41-54 |
| IPR000874 | Bombesin-like peptide | ScanRegExp | 44-50 |

Variant protein HUMGRP5E_P5 (SEQ ID NO:404) is encoded by the following transcript(s): HUMGRP5E_T5 (SEQ ID NO:392). The coding portion of transcript HUMGRP5E_T5 (SEQ ID NO:392) starts at position 622 and ends at position 1047. The transcript also has the following SNPs as listed in Table 161 (given according to their position on the nucleotide sequence, with the alternative nucleic acid listed; the last column indicates whether the SNP is known or not; the presence of known SNPs in variant protein HUMGRP5E_P5 (SEQ ID NO:404) sequence provides support for the deduced sequence of this variant protein according to the present invention).

TABLE 161
Nucleic acid SNPs

| SNP position on nucleotide | | Previously |
|---|---|---|
| sequence | Alternative nucleic acid | known SNP? |
| 541 | -> T | No |
| 542 | G -> T | No |
| 631 | A -> C | Yes |
| 672 | G -> A | Yes |
| 1354 | C -> | No |
| 1354 | C -> A | No |
| 1355 | A -> | No |
| 1355 | A -> G | No |

Table 162 below describes the starting and ending position of HUMGRP5E_node_{—}2 (SEQ ID NO:394) on each of the relevant transcripts. Experimental results for this segment are described below.

TABLE 162
Segment location on transcripts

| | Segment | Segment |
|---|---|---|
| Transcript name | starting position | ending position |
| HUMGRP5E_T1 (SEQ ID NO: 388) | 761 | 984 |
| HUMGRP5E_T2 (SEQ ID NO: 389) | 761 | 984 |
| HUMGRP5E_T4 (SEQ ID NO: 391) | 761 | 984 |
| HUMGRP5E_T5 (SEQ ID NO: 392) | 761 | 984 |

Microarray (chip) data is also available for this segment as follows. As described above with regard to the cluster itself, various oligonucleotides were tested for being differentially expressed in various disease conditions, particularly cancer. The following oligonucleotides were found to hit this segment, shown in Table 163.

TABLE 163
Oligonucleotides related to this segment

| | Overexpressed | |
|---|---|---|
| Oligonucleotide name | in cancers | Chip reference |
| HUMGRP5E_0_2_0 (SEQ ID | lung malignant tumors | LUN |
| NO: 11) | | |

Expression of Homo sapiens gastrin-releasing peptide (GRP) HUMGRP5E transcripts which are detectable by amplicon as depicted in sequence name HUMGRP5E seg2 (SEQ ID NO:407) in normal and cancerous lung tissues

Expression of Homo sapiens gastrin-releasing peptide (GRP) transcripts detectable by or according to seg2—HUMGRP5E seg2 (SEQ ID NO:407) amplicon and primers HUMGRP5E seg2F (SEQ ID NO:405) and HUMGRP5E seg2R (SEQ ID NO:406) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 5, “Tissue samples in lung cancer testing panel”), to obtain a value of fold up-regulation for each sample relative to median of the normal PM samples.

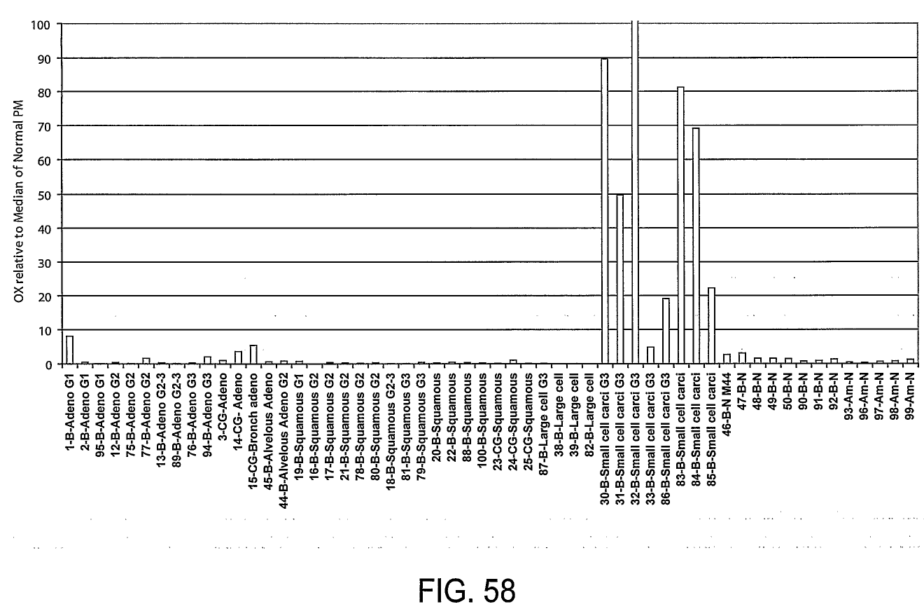

FIG. 58 is a histogram showing over expression of the above-indicated Homo sapiens gastrin-releasing peptide (GRP) transcripts in cancerous lung samples relative to the normal samples.

As is evident from FIG. 58, the expression of Homo sapiens gastrin-releasing peptide (GRP) transcripts detectable by the above amplicon in cancer samples was significantly higher than in the non-cancerous samples (Sample Nos. 46-50, 90-93, 96-99 Table 5, “Tissue samples in lung cancer testing panel”). Notably an over-expression of at least 10 fold was found in 7 out of 8 small cells carcinoma samples.

Statistical analysis was applied to verify the significance of these results, as described below.

The P value for the difference in the expression levels of Homo sapiens gastrin-releasing peptide (GRP) transcripts detectable by the above amplicon in lung small cells carcinoma samples versus the normal tissue samples was determined by T test as 425E-03.

Threshold of 10 fold overexpression was found to differentiate between lung small cells carcinoma and normal samples with P value of 1.03E-04 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: HUMGRP5E seg2F (SEQ ID NO:405) forward primer; and HUMGRP5E seg2R (SEQ ID NO:406) reverse primer.

The present invention also preferably encompasses any amplicon obtained through the use of any suitable primer pair; for example, for the above experiment, the following amplicon was obtained as a non-limiting illustrative example only of a suitable amplicon: HUMGRP5E seg2 (SEQ ID NO:407).

Primers:

Forward primer HUMGRP5E seg2F (SEQ ID NO:405):
GTGGGAAGAAGCTGCAAGGA
Reverse primer HUMGRP5E seg2R (SEQ ID NO:406):
CCACGAAGGCTGCTGATTG
Amplicon HUMGRP5E seg2 (SEQ ID NO:407):
GTGGGAAGAAGCTGCAAGGAATTTGCTGGGTCTCATAGAAGCAAAGGAGA
ACAGAAACCACCAGCCACCTCAACCCAAGGCCCTGGGCAATCAGCAGCCT
TCGTGG

Expression of Homo sapiens gastrin-releasing peptide (GRP) HUMGRP5E transcripts which are detectable by amplicon as depicted in sequence name HUMGRP5E seg2 (SEQ ID NO: 407) in different normal tissues

Expression of Homo sapiens gastrin-releasing peptide (GRP) transcripts detectable by or according to HUMGRP5E seg2 (SEQ ID NO: 407) amplicon and primers: HUMGRP5E seg2F (SEQ ID NO: 405) and HUMGRP5E seg2Rseg2F (SEQ ID NO: 406) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the breast samples (Sample Nos. 33-35 Table 7, “Tissue samples in normal panel”), to obtain a value of relative expression of each sample relative to median of the breast samples.

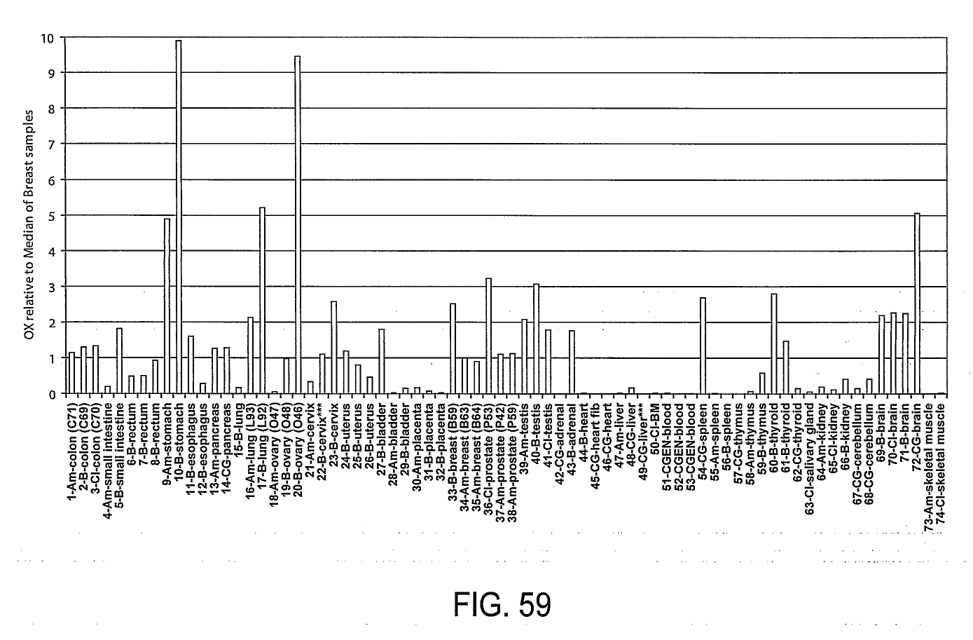

FIG. 59 is a histogram showing Expression of Homo sapiens gastrin-releasing peptide (GRP) HUMGRP5E transcripts which are detectable by amplicon as depicted in sequence name HUMGRP5E seg2 (SEQ ID NO: 407) in different normal tissues

Primers:

Forward primer HUMGRP5E seg2F (SEQ ID NO:405):
GTGGGAAGAAGCTGCAAGGA
Reverse primer HUMGRP5E seg2R (SEQ ID NO:406):
CCACGAAGGCTGCTGATTG
Amplicon HUMGRP5E seg2 (SEQ ID NO:407):
GTGGGAAGAAGCTGCAAGGAATTTGCTGGGTCTCATAGAAGCAAAGGAGA
ACAGAAACCACCAGCCACCTCAACCCAAGGCCCTGGGCAATCAGCAGCCT
TCGTGG

Expression of Homo sapiens gastrin-releasing peptide (GRP) HUMGRP5E transcripts which are detectable by amplicon as depicted in sequence name HUMGRP5E seg2 (SEQ ID NO:407) in normal and cancerous breast tissues

Expression of Homo sapiens gastrin-releasing peptide (GRP) transcripts detectable by or according to seg2—HUMGRP5E seg2 (SEQ ID NO:407) amplicon and primers HUMGRP5E seg2F (SEQ ID NO:405) and HUMGRP5E seg2R (SEQ ID NO:406) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and G6PD (GenBank Accession No. NM_{—}000402 (SEQ ID NO:8); G6PD amplicon (SEQ ID NO: 44)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67, Table 6, above, “Tissue samples in breast cancer testing sample”), to obtain a value of fold up-regulation for each sample relative to median of the normal PM samples.

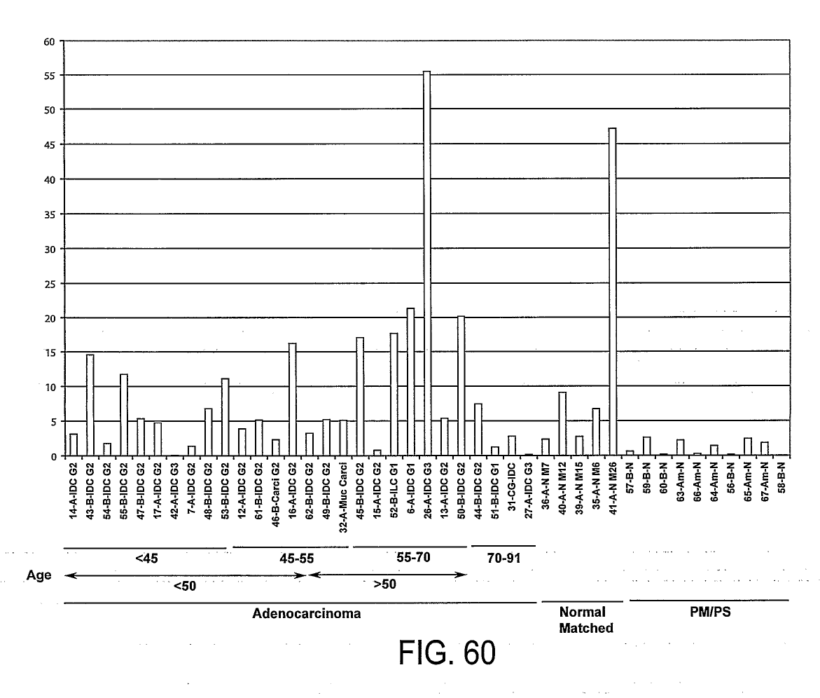

FIG. 60 is a histogram showing over expression of the above-indicated Homo sapiens gastrin-releasing peptide (GRP) transcripts in cancerous breast samples relative to the normal samples.

As is evident from FIG. 60, the expression of Homo sapiens gastrin-releasing peptide (GRP) transcripts detectable by the above amplicon in cancer samples was significantly higher than in the non-cancerous samples (Sample Nos. 56-60, 63-67 Table 6, “Tissue samples in breast cancer testing sample”). Notably an over-expression of at least 5 fold was found in 16 out of 28 adenocarcinoma samples.

Statistical analysis was applied to verify the significance of these results, as described below. The P value for the difference in the expression levels of Homo sapiens gastrin-releasing peptide (GRP) transcripts detectable by the above amplicon in breast cancer samples versus the normal tissue samples was determined by T test as 1.02E-03.

Threshold of 5 fold overexpression was found to differentiate between cancer and normal samples with P value of 1.37E-03 as checked by exact fisher test. The above values demonstrate statistical significance of the results.

By dissecting the patient's population into two age groups (indicated below the graph), we demonstrate that the transcripts detected by the above amplicon are highly expressed mainly in patients with an age above 55.

Primer pairs are also optionally and preferably encompassed within the present invention; for example, for the above experiment, the following primer pair was used as a non-limiting illustrative example only of a suitable primer pair: HUMGRP5E seg2F (SEQ ID NO:405) forward primer; and HUMGRP5E seg2R (SEQ ID NO:406) reverse primer.

The present invention also preferably encompasses any amplicon obtained through the use of any suitable primer pair; for example, for the above experiment, the following amplicon was obtained as a non-limiting illustrative example only of a suitable amplicon: HUMGRP5E seg2 (SEQ ID NO:407).

Primers:

Forward primer HUMGRP5E seg2F (SEQ ID NO:405):
GTGGGAAGAAGCTGCAAGGA
Reverse primer HUMGRP5E seg2R (SEQ ID NO:406):
CCACGAAGGCTGCTGATTG
Amplicon HUMGRP5E seg2 (SEQ ID NO:407):
GTGGGAAGAAGCTGCAAGGAATTTGCTGGGTCTCATAGAAGCAAAGGAGA
ACAGAAACCACCAGCCACCTCAACCCAAGGCCCTGGGCAATCAGCAGCCT
TCGTGG

Expression of Homo sapiens gastrin-releasing peptide (GRP) HUMGRP5E transcripts which are detectable by amplicon as depicted in sequence name HUMGRP5E seg2 (SEQ ID NO:407) in normal and cancerous ovary tissues

Expression of Homo sapiens gastrin-releasing peptide (GRP) transcripts detectable by or according to seg2—HUMGRP5E seg2 (SEQ ID NO:407) amplicon and primers HUMGRP5E seg2F (SEQ ID NO:405) and HUMGRP5E seg2R (SEQ ID NO:406) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold up-regulation for each sample relative to median of the normal PM samples.

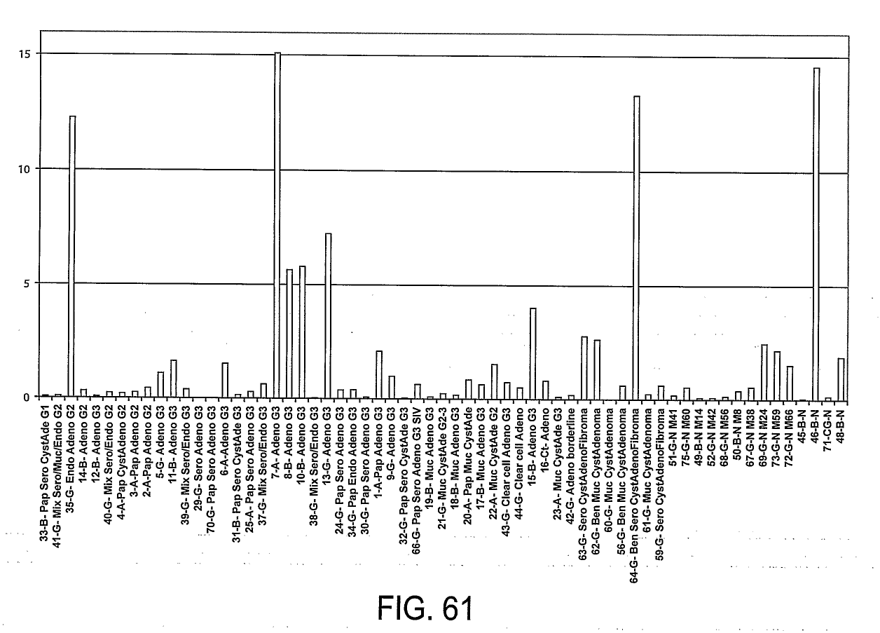

FIG. 61 is a histogram showing over expression of the above-indicated Homo sapiens gastrin-releasing peptide (GRP) transcripts in cancerous ovary samples relative to the normal samples median. As is evident from FIG. 61, the expression of GRP_HUMAN (SEQ ID NO:400)—gastrin-releasing peptide transcripts detectable by the above amplicon was higher in a few cancer samples than in most of the non-cancerous samples (Sample Nos. 45, 48, 71 Table 3, above, “Tissue samples in ovarian cancer testing panel”).

Notably an over-expression of at least 5 fold was found in 5 out of 43 adenocarcinoma samples.

Primer pairs are also optionally and preferably encompassed within the present invention; for example, for the above experiment, the following primer pair was used as a non-limiting illustrative example only of a suitable primer pair: HUMGRP5E seg2F (SEQ ID NO:405) forward primer; and HUMGRP5E seg2R (SEQ ID NO:406) reverse primer.

The present invention also preferably encompasses any amplicon obtained through the use of any suitable primer pair; for example, for the above experiment, the following amplicon was obtained as a non-limiting illustrative example only of a suitable amplicon: HUMGRP5E seg2 (SEQ ID NO:407).

Primers:

Forward primer HUMGRP5E seg2F (SEQ ID NO:405):
GTGGGAAGAAGCTGCAAGGA
Reverse primer HUMGRP5E seg2R (SEQ ID NO:406):
CCACGAAGGCTGCTGATTG
Amplicon HUMGRP5E seg2 (SEQ ID NO:407):
GTGGGAAGAAGCTGCAAGGAATTTGCTGGGTCTCATAGAAGCAAAGGAGA
ACAGAAACCACCAGCCACCTCAACCCAAGGCCCTGTGGCAATCAGCAGCC
TTCGTGG

Expression of GRP_HUMAN (SEQ ID NO:400)—gastrin-releasing peptide (HUMGRP5E) transcripts, which are detectable by amplicon, as depicted in sequence name HUMGRP5Ejunc3-7 (SEQ ID NO: 410) in normal and cancerous breast tissues.

Expression of GRP_HUMAN (SEQ ID NO:400)—gastrin-releasing peptide transcripts detectable by or according to junc3-7, HUMGRP5Ejunc3-7 (SEQ ID NO: 410) amplicon and HUMGRP5Ejunc3-7F (SEQ ID NO: 408) and HUMGRP5Ejunc3-7R (SEQ ID NO: 409) primers was measured by real time PCR. In parallel the expression of four housekeeping genes PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), and SDHA (GenBank Accession No. NM_{—}004168 (SEQ ID NO:4); amplicon—SDHA-amplicon (SEQ ID NO:29)), G6PD (GenBank Accession No. NM_{—}000402 (SEQ ID NO:8); G6PD amplicon (SEQ ID NO: 44)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67 Table 6, “Tissue samples in breast cancer testing panel”), to obtain a value of fold up-regulation for each sample relative to median of the normal PM samples.

FIG. 5 is a histogram showing over expression of the above-indicated GRP_HUMAN (SEQ ID NO:400)—gastrin-releasing peptide transcripts in cancerous breast samples relative to the normal samples. Values represent the average of duplicate experiments. Error bars indicate the minimal and maximal values obtained. The number and percentage of samples that exhibit at least 62 fold over-expression, out of the total number of samples tested is indicated in the bottom.

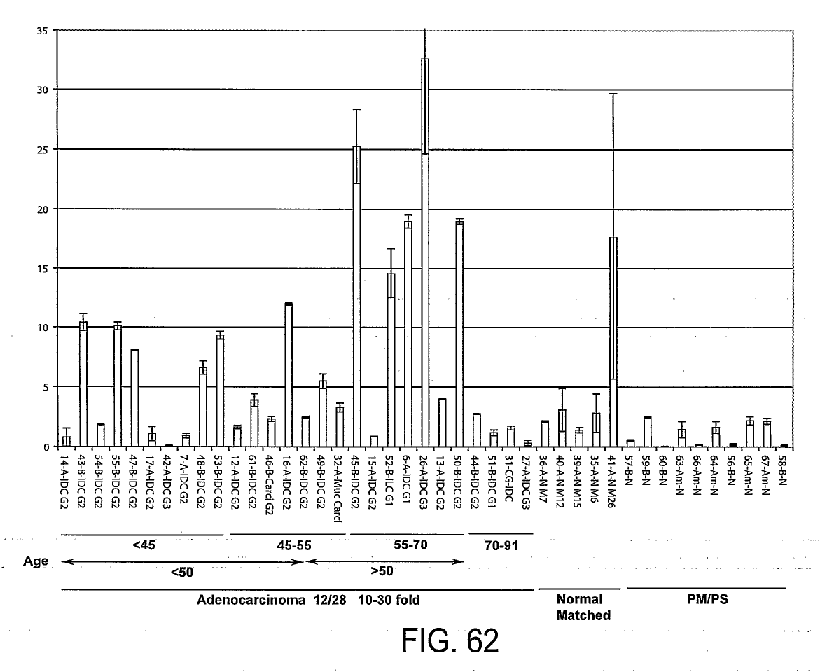

As is evident from FIG. 62, the expression of GRP_HUMAN (SEQ ID NO:400)—gastrin-releasing peptide transcripts detectable by the above amplicon in cancer samples was significantly higher than in the non-cancerous samples (Sample Nos., 56-60, 63-67 table 6, “Tissue samples in breast cancer testing panel”). Notably an over-expression of at least 5 fold was found in 12 out of 28 adenocarcinoma samples. Statistical analysis was applied to verify the significance of these results, as described below.
The P value for the difference in the expression levels of GRP_HUMAN (SEQ ID NO:400)—gastrin-releasing peptide transcripts detectable by the above amplicon in breast cancer samples versus the normal tissue samples was determined by T test as 7.22E-04. Threshold of 5 fold over expression was found to differentiate between cancer and normal samples with P value of 1.12E-02 as checked by exact fisher test. The above values demonstrate statistical significance of the results.
By dissecting the cancer patient's population into two age groups (indicated below the graph), we demonstrate that the transcripts detected by the above amplicon are highly expressed mainly in patients with an age above 55.
Primer pairs are also optionally and preferably encompassed within the present invention; for example, for the above experiment, the following primer pair was used as a non-limiting illustrative example only of a suitable primer pair: HUMGRP5Ejunc3-7F (SEQ ID NO: 408) forward primer; and HUMGRP5Ejunc3-7R (SEQ ID NO: 409) reverse primer.
The present invention also preferably encompasses any amplicon obtained through the use of any suitable primer pair; for example, for the above experiment, the following amplicon was obtained as a non-limiting illustrative example only of a suitable amplicon: HUMGRP5Ejunc3-7 (SEQ ID NO: 410).

HUMGRP5Ejunc3-7F (SEQ ID NO:408):
ACCAGCCACCTCAACCCA
HUMGRP5Ejunc3-7R (SEQ ID NO:409):
CTGGAGCAGAGAGTCTTTGCCT
HUMGRP5Ejunc3-7 (SEQ ID NO:410):
ACCAGCCACCTCAACCCAAGGCCCTGGGCAATCAGCAGCCTTCGTGGGAT
TCAGAGGATAGCAGCAACTTCAAAGATGTAGGTTCAAAAGGCAAAGACTC
TCTGCTCCAG

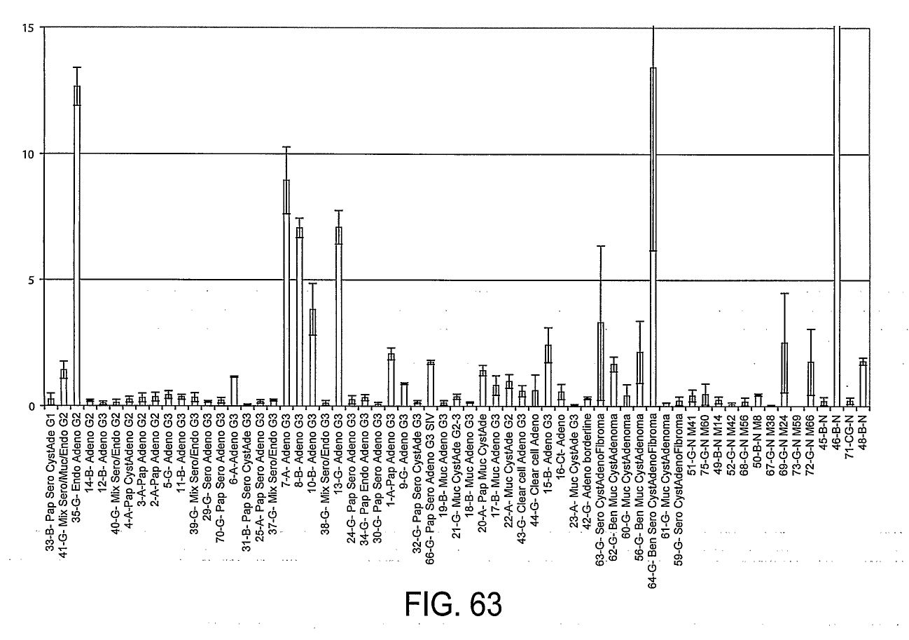

Expression of GRP_HUMAN (SEQ ID NO:400)—gastrin-releasing peptide HUMGRP5E transcripts, which are detectable by amplicon as depicted in sequence name HUMGRP5E junc3-7 (SEQ ID NO: 410) in normal and cancerous ovary tissues.
Expression of GRP_HUMAN (SEQ ID NO:400)—gastrin-releasing peptide transcripts detectable by or according to HUMGRP5Ejunc3-7 (SEQ ID NO: 410) amplicon and HUMGRP5Ejunc3-7F (SEQ ID NO: 408) and HUMGRP5Ejunc3-7R (SEQ ID NO: 409) primers was measured by real time PCR. In parallel the expression of four housekeeping genes PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon and SDHA (GenBank Accession No. NM_{—}004168 (SEQ ID NO:4); amplicon—SDHA-amplicon (SEQ ID NO:29)), GAPDH (GenBank Accession No. BC026907 (SEQ ID NO:3); GAPDH amplicon (SEQ ID NO: 41)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, “Tissue samples in ovarian cancer testing panel”), to obtain a value of fold up-regulation for each sample relative to median of the normal PM samples.
FIG. 63 is a histogram showing over expression of the above-indicated GRP_HUMAN (SEQ ID NO:400)—gastrin-releasing peptide transcripts in cancerous ovary samples relative to the normal samples. Values represent the average of duplicate experiments. Error bars indicate the minimal and maximal values obtained.
As is evident from FIG. 63 the expression of GRP_HUMAN (SEQ ID NO:400)—gastrin-releasing peptide transcripts detectable by the above amplicon was higher in a few cancer samples than in most of the non-cancerous samples (Sample Nos. 45, 48, 71, Table 3, “Tissue samples in ovarian cancer testing panel”). Notably an over-expression of at least 5-fold was found in 4 out of 43 adenocarcinoma samples,

Primer pairs are also optionally and preferably encompassed within the present invention; for example, for the above experiment, the following primer pair was used as a non-limiting illustrative example only of a suitable primer pair: HUMGRP5Ejunc3-7F (SEQ ID NO: 408) forward primer; and HUMGRP5Ejunc3-7R (SEQ ID NO: 409) reverse primer.

The present invention also preferably encompasses any amplicon obtained through the use of any suitable primer pair; for example, for the above experiment, the following amplicon was obtained as a non-limiting illustrative example only of a suitable amplicon: HUMGRP5Ejunc3-7 (SEQ ID NO: 410).

HUMGRP5Ejunc3-7F (SEQ ID NO:408)
ACCAGCCACCTCAACCCA
HUMGRP5Ejunc3-7R (SEQ ID NO:409)
CTGGAGCAGAGAGTCTTTGCCT
HUMGRP5Ejunc3-7 (SEQ ID NO:410)
ACCAGCCACCTCAACCCAAGGCCCTGGGCAATCAGCAGCCTTCGTGGGAT
TCAGAGGATAGCAGCAACTTCAAAGATGTAGGTTCAAAAGGCAAAGACTC
TCTGCTCCAG

Expression of GRP_HUMAN (SEQ ID NO:400)—gastrin-releasing peptide HUMGRP5E transcripts, which are detectable by amplicon as depicted in sequence name HUMGRP5E junc3-7 (SEQ ID NO: 410) in normal and cancerous colon tissues.

Expression of GRP_HUMAN (SEQ ID NO:400)—gastrin-releasing peptide transcripts detectable by or according to HUMGRP5Ejunc3-7 (SEQ ID NO: 410) amplicon and HUMGRP5Ejunc3-7F (SEQ ID NO: 408) and HUMGRP5Ejunc3-7R (SEQ ID NO: 409) primers was measured by real time PCR. In parallel the expression of four housekeeping genes PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), and, G6PD (GenBank Accession No. NM_{—}000402 (SEQ ID NO:8); G6PD amplicon (SEQ ID NO: 44)), RPS27A (GenBank Accession No. NM_{—}002954 (SEQ ID NO:1); RPS27A amplicon (SEQ ID NO: 47)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 4, “Tissue samples in colon cancer testing panel”), to obtain a value of fold differential expression for each sample relative to median of the normal PM samples.

In one experiment that was carried out no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: HUMGRP5Ejunc3-7F (SEQ ID NO: 408) forward primer; and HUMGRP5Ejunc3-7R (SEQ ID NO: 409) reverse primer.

The present invention also preferably encompasses any amplicon obtained through the use of any suitable primer pair; for example, for the above experiment, the following amplicon was obtained as a non-limiting illustrative example only of a suitable amplicon: HUMGRP5Ejunc3-7 (SEQ ID NO: 410).

HUMGRP5Ejunc3-7F (SEQ ID NO:408)
ACCAGCCACCTCAACCCA
HUMGRP5Ejunc3-7R (SEQ ID NO:409)
CTGGAGCAGAGAGTCTTTGCCT
HUMGRP5Ejunc3-7 (SEQ ID NO:410)
ACCAGCCACCTCAACCCAAGGCCCTGGGCAATCAGCAGCCTTCGTGGGAT
TCAGAGGATAGCAGCAACTTCAAAGATGTAGGTTCAAAAGGCAAAGACTC
TCTGCTCCAG

Expression of GRP_HUMAN (SEQ ID NO:400)—gastrin-releasing peptide HUMGRP5E transcripts, which are detectable by amplicon as depicted in sequence name HUMGRP5Ejunc3-7 (SEQ ID NO: 410) in normal and cancerous lung tissues

Expression of GRP_HUMAN (SEQ ID NO:400)—gastrin-releasing peptide transcripts detectable by or according to HUMGRP5Ejunc3-7 (SEQ ID NO: 410) amplicon and HUMGRP5Ejunc3-7F (SEQ ID NO: 408) and HUMGRP5Ejunc3-7R (SEQ ID NO: 409) primers was measured by real time PCR. In parallel the expression of four housekeeping genes PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), UBC (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 5, “Tissue samples in lung cancer testing panel”), to obtain a value of fold up-regulation for each sample relative to median of the normal PM samples.

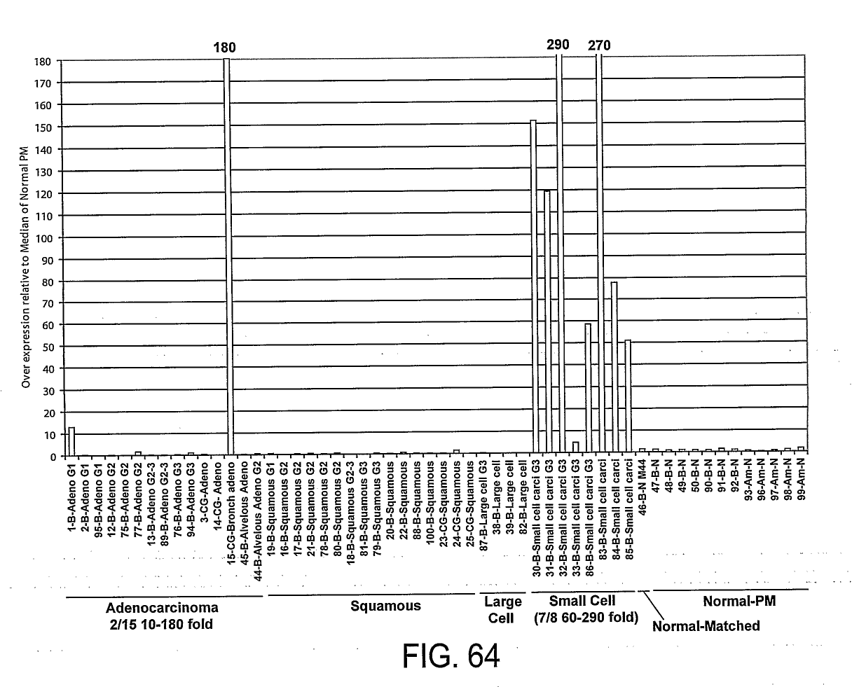

FIG. 64 is a histogram showing over expression of the above-indicated GRP_HUMAN (SEQ ID NO:400)—gastrin-releasing peptide transcripts in cancerous lung samples relative to the normal samples. The number of samples that exhibit at least 10-fold over-expression, out of the total number of samples tested is indicated in the bottom.
As is evident from FIG. 64, the expression of GRP_HUMAN (SEQ ID NO:400)—gastrin-releasing peptide transcripts detectable by the above amplicon in cancer samples was significantly higher than in the non-cancerous samples (Sample Nos. 47-50, 90-93, 96-99, Table 5, “Tissue samples in lung cancer testing panel”). Notably an over-expression of at least 10 fold was found in 2 out of 15 adenocarcinoma samples, and in 7 out of 8 small cells carcinoma samples.

Statistical analysis was applied to verify the significance of these results, as described below.

The P value for the difference in the expression levels of GRP_HUMAN (SEQ ID NO:400)—gastrin-releasing peptide transcripts detectable by the above amplicon in lung cancer samples versus the normal tissue samples was determined by T test as 3.23E-01 in adenocarcinoma cancer and 5.24E-03 in small cell carcinoma.

Threshold of 10 fold over expression was found to differentiate between cancer and normal samples with value of 2.99E-01 in adenocarcinoma cancer and Threshold of 50 fold over expression was found to differentiate between cancer and normal samples with P value of 1.03E-04 in small cell carcinoma as checked by exact fisher test. The above values demonstrate statistical significance of the results. Primer pairs are also optionally and preferably encompassed within the present invention; for example, for the above experiment, the following primer pair was used as a non-limiting illustrative example only of a suitable primer pair: HUMGRP5Ejunc3-7F (SEQ ID NO: 408) forward primer; and HUMGRP5Ejunc3-7R (SEQ ID NO: 409) reverse primer.

The present invention also preferably encompasses any amplicon obtained through the use of any suitable primer pair; for example, for the above experiment, the following amplicon was obtained as a non-limiting illustrative example only of a suitable amplicon: HUMGRP5Ejunc3-7 (SEQ ID NO: 410).

HUMGRP5Ejunc3-7F (SEQ ID NO:408)
ACCAGCCACCTCAACCCA
HUMGRP5Ejunc3-7R (SEQ ID NO:409)
CTGGAGCAGAGAGTCTTTGCCT
HUMGRP5Ejunc3-7 (SEQ ID NO:410)
ACCAGCCACCTCAACCCAAGGCCCTGGGCAATCAGCAGCCTTCGTGGGAT
TCAGAGGATAGCAGCAACTTCAAAGATGTAGGTTCAAAAGGCAAAGACTC
TCTGCTCCAG

Expression of GRP_HUMAN (SEQ ID NO:400)—gastrin-releasing peptide HUMGRP5E transcripts, which are detectable by amplicon as depicted in sequence name HUMGRP5E junc3-7 (SEQ ID NO: 410) in different normal tissues.

Expression of GRP_HUMAN (SEQ ID NO:400)—gastrin-releasing peptide transcripts detectable by or according to HUMGRP5E junc3-7 (SEQ ID NO: 410) amplicon and HUMGRP5E junc3-7F (SEQ ID NO:408) and HUMGRP5E junc3-7R (SEQ ID NO:409) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), UBC (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the breast samples (Sample Nos. 33-35 Table 7, “Tissue samples in normal panel”), to obtain a value of relative expression of each sample relative to median of the breast samples.

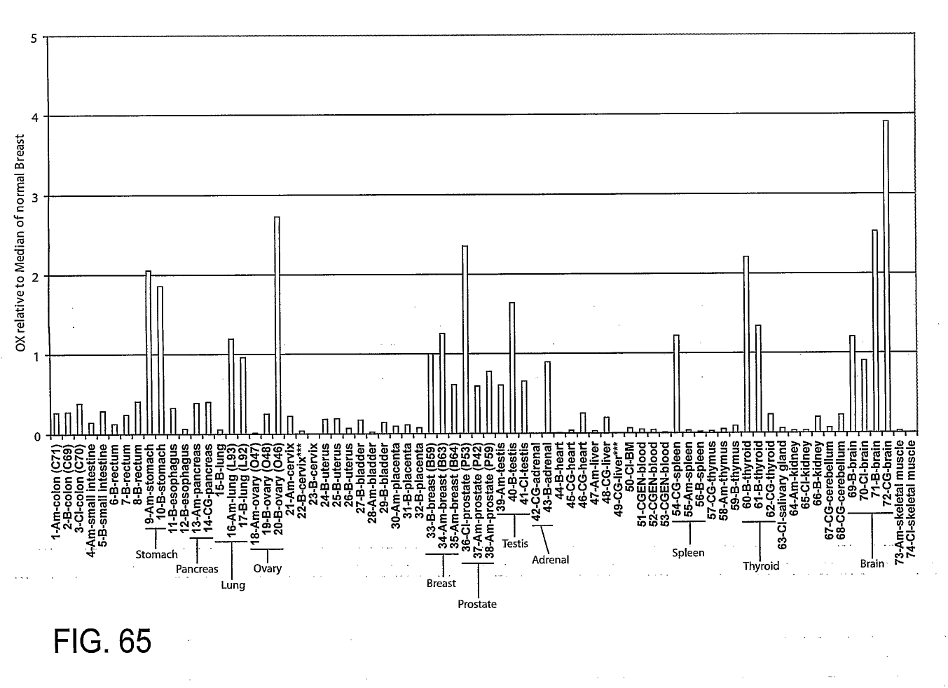

FIG. 65 is a histogram showing expression of GRP_HUMAN (SEQ ID NO:400)—gastrin-releasing peptide HUMGRP5E transcripts, which are detectable by amplicon as depicted in sequence name HUMGRP5E junc3-7 (SEQ ID NO: 410) in different normal tissues.

Forward primer HUMGRP5Ejunc3-7F (SEQ ID NO:408):
ACCAGCCACCTCAACCCA
Reverse primer HUMGRP5Ejunc3-7R (SEQ ID NO:409):
CTGGAGCAGAGAGTCTTTGCCT
Amplicon HUMGRP5Ejunc3-7 (SEQ ID NO:410):
ACCAGCCACCTCAACCCAAGGCCCTGGGCAATCAGCAGCCTTCGTGGGAT
TCAGAGGATAGCAGCAACTTCAAAGATGTAGGTTCAAAAGGCAAAGACTC
TCTGCTCCAG

### Description for Cluster T94936

Cluster T94936 features 3 transcript(s) and 12 segment(s) of interest, the names for which are given in Tables 164 and 165. The selected protein variants are given in table 166.

TABLE 164
Transcripts of interest
Transcript Name

T94936_PEA_1_PEA_1_T1 (SEQ ID NO: 411)
T94936_PEA_1_PEA_1_T2 (SEQ ID NO: 412)
T94936_PEA_1_PEA_1_T5 (SEQ ID NO: 413)

TABLE 165
Segments of interest

| Segment Name | |
|---|---|
| T94936_PEA_1_PEA_1_node_2 (SEQ ID NO: 414) | T94936_PEA_1_PEA_1_T1 (SEQ ID |
| | NO: 411), T94936_PEA_1_PEA_1_T2 |
| | (SEQ ID NO: 412) and |
| | T94936_PEA_1_PEA_1_T5 (SEQ ID |
| | NO: 413) |
| T94936_PEA_1_PEA_1_node_14 (SEQ ID NO: 415) | T94936_PEA_1_PEA_1_T2 (SEQ ID |
| | NO: 412) |
| T94936_PEA_1_PEA_1_node_16 (SEQ ID NO: 416) | T94936_PEA_1_PEA_1_T2 (SEQ ID |
| | NO: 412) |
| T94936_PEA_1_PEA_1_node_20 (SEQ ID NO: 417) | T94936_PEA_1_PEA_1_T2 (SEQ ID |
| | NO: 412) and T94936_PEA_1_PEA_1_T5 |
| | (SEQ ID NO: 413) |
| T94936_PEA_1_PEA_1_node_23 (SEQ ID NO: 418) | T94936_PEA_1_PEA_1_T1 (SEQ ID |
| | NO: 411) |
| T94936_PEA_1_PEA_1_node_0 (SEQ ID NO: 419) | T94936_PEA_1_PEA_1_T1 (SEQ ID |
| | NO: 411), T94936_PEA_1_PEA_1_T2 |
| | (SEQ ID NO: 412) and |
| | T94936_PEA_1_PEA_1_T5 (SEQ ID |
| | NO: 413) |
| T94936_PEA_1_PEA_1_node_6 (SEQ ID NO: 420) | T94936_PEA_1_PEA_1_T1 (SEQ ID |
| | NO: 411), T94936_PEA_1_PEA_1_T2 |
| | (SEQ ID NO: 412) and |
| | T94936_PEA_1_PEA_1_T5 (SEQ ID |
| | NO: 413) |
| T94936_PEA_1_PEA_1_node_8 (SEQ ID NO: 421) | T94936_PEA_1_PEA_1_T1 (SEQ ID |
| | NO: 411) and T94936_PEA_1_PEA_1_T2 |
| | (SEQ ID NO: 412) |
| T94936_PEA_1_PEA_1_node_9 (SEQ ID NO: 422) | T94936_PEA_1_PEA_1_T1 (SEQ ID |
| | NO: 411), T94936_PEA_1_PEA_1_T2 |
| | (SEQ ID NO: 412) and |
| | T94936_PEA_1_PEA_1_T5 (SEQ ID |
| | NO: 413) |
| T94936_PEA_1_PEA_1_node_11 (SEQ ID NO: 423) | T94936_PEA_1_PEA_1_T1 (SEQ ID |
| | NO: 411), T94936_PEA_1_PEA_1_T2 |
| | (SEQ ID NO: 412) and |
| | T94936_PEA_1_PEA_1_T5 (SEQ ID |
| | NO: 413) |
| T94936_PEA_1_PEA_1_node_13 (SEQ ID NO: 424) | T94936_PEA_1_PEA_1_T1 (SEQ ID |
| | NO: 411), T94936_PEA_1_PEA_1_T2 |
| | (SEQ ID NO: 412) and |
| | T94936_PEA_1_PEA_1_T5 (SEQ ID |
| | NO: 413) |
| T94936_PEA_1_PEA_1_node_17 (SEQ ID NO: 425) | T94936_PEA_1_PEA_1_T1 (SEQ ID |
| | NO: 411), T94936_PEA_1_PEA_1_T2 |
| | (SEQ ID NO: 412) and |
| | T94936_PEA_1_PEA_1_T5 (SEQ ID |
| | NO: 413) |

TABLE 166
Proteins of interest

| Protein Name | Corresponding Transcript(s) |
|---|---|
| T94936_PEA_1_PEA_1_P2 | T94936_PEA_1_PEA_1_T1 |
| (SEQ ID NO: 427) | (SEQ ID NO: 411) |
| T94936_PEA_1_PEA_1_P3 | T94936_PEA_1_PEA_1_T2 |
| (SEQ ID NO: 428) | (SEQ ID NO: 412) |
| T94936_PEA_1_PEA_1_P7 | T94936_PEA_1_PEA_1_T5 |
| (SEQ ID NO: 429) | (SEQ ID NO: 413) |

These sequences are variants of the known protein Anterior gradient protein 3 (SwissProt accession identifier Q8TD06_HUMAN (SEQ ID NO:695); known also according to the synonyms MLHS642, Breast cancer membrane protein 11, BCMP11, HAG3, hAG3), referred to herein as the previously known protein.

According to optional but preferred embodiments of the present invention, variants of this cluster according to the present invention (amino acid and/or nucleic acid sequences of T94936) may optionally have one or more of the following utilities, as described with regard to Table 167 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 167
Utilities for Variants of T94936

| | | |
|---|---|---|
| 1. Targets for breast | Shows cancer cell membrane association as well | Adam PJ, J Biol |
| cancer therapy and/or | as protein expression levels and localizations that are | Chem. 2003 Feb |
| diagnostic markers of | unique to cancer cells over normal breast epithelial cells. | 21; 278(8): 6482-9. |
| the disease | BCMP11/hAG3 shows the highest prevalence in | Epub 2002 Dec 10 |
| | breast cancers (74%) with a strong positive correlation with | |
| | estrogen receptor status. no mRNA or protein expression in | |
| | normal breast epithelia or stromal tissue | |
| | C4.4A, as a BCMP11-interacting protein. C4.4A | |
| | may therefore represent an autocrine receptor for BCMP11. | |
| 2. hAG-2 and hAG-3 | Yeast two-hybrid cloning identified metastasis-associated | Fletcher GC et al., |
| are human | GPI-anchored C4.4a protein and extracellular alpha- | Br J Cancer. 2003 |
| homologues of the | dystroglycan (DAG-1) as binding partners for both hAG-2 | Feb 24; 88(4): 579-85 |
| secreted Xenopus | and hAG-3, which if replicated in clinical oncology would | |
| laevis proteins XAG- | demonstrate a potential role in tumour metastasis through | |
| ½ (AGR-½); | the regulation of receptor adhesion and functioning. hAG-2 | |
| Breast tumor | and hAG-3 may therefore serve as useful molecular | |
| biomarkers | markers and/or potential therapeutic targets for hormone- | |
| | responsive breast tumors. | |
| 3. anterior gradient 2 | Serial analysis of gene expression and more recently | Missiaglia E., Int J |
| (AG2) (high | oligo/cDNA microarray technologies have been employed | Cancer. 2004 Oct |
| homology with AG3, | in order to identify genes involved in pancreatic neoplasia | 20; 112(1): 100-12 |
| lie adjacent to it) | that can be developed as diagnostic markers and drug | |
| Pancreatic cancer | targets for this dismal disease. | |
| 4. Co-expression of | hAG-2 has been found to be co-expressed with estrogen | Thompson DA et |
| hAG-2 with ER | receptor (ER) in breast cancer cell lines by using | al., Biochem |
| suggests that hAG-2 | suppression subtractive hybridization | Biophys Res |
| may be involved in | | Commun. 1998 Oct |
| the tumor biology | | 9; 251(1): 111-6 |
| specific to the well- | | |
| differentiated | | |
| phenotype of | | |
| hormonally- | | |
| responsive breast | | |
| cancers. | | |
| 5. C4.4A, a member | C4.4A was described as a metastasis-associated molecule. | Paret C et al., Int J |
| of the Ly6 family. It | C4.4A ligands are strongly expressed in tissues adjacent to | Cancer. 2005 Feb |
| has been detected | squamous epithelia of, e.g., tongue and esophagus, the | 23 |
| mainly on | expression pattern partly overlapping with laminin (LN) | |
| metastasizing | and complementing the C4.4A expression that is found | |
| carcinoma cells and | predominantly on the basal layers of squamous epithelium | |
| proposed to be | LN1 and LN5 are C4.4A ligands that galectin-3 associates | |
| involved in wound | with C4.4A and that C4.4A ligand binding confers a | |
| healing | migratory phenotype are well in line with the supposed | |
| | metastasis association. | |
| 6. MIG-C4-the | Gene products involved in normal urothelium-matrix | Smith BA et al., |
| human homologue of | interactions that could be tumor-invasion or suppressor- | Cancer Res. 2001 |
| the rat metastasis- | gene targets in the development of invasive and metastatic | Feb 15; 61(4): 1678-85 |
| associated C4.4A | tumor phenotypes. | |
| gene-malignant | | |
| progression | | |
| 7. Possible | Functional activity of hC4.4 A in tumor progression. | Seiter S et al., J |
| correlation between | Melanoma lines were incubated with human serum. | Invest Dermatol. |
| hC4.4 A and tumor | Whereas expression of hC4.4 was not influenced by heat- | 2001 |
| progression | inactivated human serum, all melanoma lines responded to | Feb; 116(2): 344-7 |
| | non inactivated human serum with up regulation of hC4.4 | |
| | A expression. Regulated expression with highest-level | |
| | expression on metastases is a feature that hC4.4 A shares | |
| | with the urokinase-type plasminogen activator receptor. | |
| 8. C4.4A with tumor | Expression of the human C4.4A was observed by RT-PCR | Wurfel J, et al |
| progression, human | and Northern blotting in placental tissue, skin, esophagus | Gene. 2001 Jan |
| C4.4A might well | and peripheral blood leukocytes, but not in brain, lung, | 10; 262(1-2): 35-41 |
| become a prognostic | liver, kidney, stomach, colon and lymphoid organs. Yet, | |
| marker and possibly | tumors derived from the latter tissues frequently contained | |
| a target of therapy. | C4.4A mRNA. As demonstrated for malignant melanoma, | |
| | C4.4A mRNA expression correlated with tumor | |
| | progression. While nevi were negative and only a minority | |
| | of primary malignant melanoma expressed C4.4A, all | |
| | metastases were C4.4A-positive | |
| marker for the | huXAG-1 is specifically found in cancerous colon cells and | WO9807749 |
| detection of | may therefore be a growth factor for colon cancer. In | |
| colorectal carcinoma: | particular, isolated nucleic acid molecules are provided | |
| a huXAG-1, | encoding the huXAG-1, huXAG-2 and huXAG-3 proteins. | |
| huXAG-2 and | | |
| huXAG-3 proteins | | |
| which are novel | | |
| human growth | | |
| factors. These | | |
| proteins share | | |
| homology with the | | |
| XAG protein of | | |
| Xenopus laevis and | | |
| can be found in colon | | |
| tissue | | |
| Involved in protein- | BCMP 11 is useful for the diagnosis, screening, treatment | WO03075014 |
| protein interactions | and prophylaxis of breast cancer, as well as compositions | |
| involving Breast | comprising BCMP 11, including vaccines and antibodies | |
| Cancer Membrane | that are immunospecific for BCMP 11. | |
| Proteins-7 and -11 | Human nucleic acid sequences - mRNA, cDNA, genome | |
| (BCMP-7 and | sequences - of ovarian tumor tissue, which code for gene | |
| BCMP-11). relates to | products or parts of these products, and to their use. Also | |
| complexes of the | polypeptides obtained by way of these sequences and to the | |
| interacting proteins, | use of same. | |
| antibodies to the | | |
| complexes, selected | | |
| interaction domains | | |
| of the protein-protein | | |
| interactions, methods | | |
| for screening for | | |
| agents which | | |
| modulate the protein- | | |
| protein interactions | | |
| and pharmaceutical | | |
| compositions | | |
| comprising active | | |
| agents that are | | |
| capable of | | |
| modulating the | | |
| protein-protein | | |
| interactions | | |

According to other optional embodiments of the present invention, variants of this cluster according to the present invention (amino acid and/or nucleic acid sequences of T94936) may optionally have one or more of the following utilities, some of which are related to utilities described above. It should be noted that these utilities are optionally and preferably suitable for human and non-human animals as subjects, except where otherwise noted.

A non-limiting example of such a utility is screening for and/or diagnosis of breast cancer in a subject, and/or disease prognosis, and/or monitoring the effectiveness of breast cancer therapy, by using at least one T94936 variant according to the present invention. Optionally and preferably, the method involves detecting a change in level and/or activity of the at least one variant.

Such use of the known protein is described with regard to PCT Application No. WO 03/087831, hereby incorporated by reference as if fully set forth herein.

Cluster T94936 belongs to a family of proteins which are known to have functions related to the cardiovascular system and functions, including but not limited to, AGR2. These functions are described below; one or more variants of cluster T94936 may optionally have one or more diagnostic utilities related to these functions.

Changes in AGR2 expression were indicated in breast cancer (Shen D, et al.: Loss of annexin A1 expression in human breast cancer detected by multiple high-throughput analyses. Biochem Biophys Res Commun. 2005 Jan. 7; 326(1):218-27.). AGR2 was found to be over-expressed in 89% of prostate carcinomas, but did not have prognostic significance (Kristiansen G, et al.: Expression profiling of microdissected matched prostate cancer samples reveals CD166/MEMD and CD24 as new prognostic markers for patient survival. J Pathol. 2005 February; 205(3):359-76.). However, one or more variants of T94936 may optionally have such prognostic significance.

It was identified as a reliable marker of borderline tumors. Interestingly, two of these genes, AGR2 and NPTX2, are physically linked to one another, mapping to p21.3 on chromosome 7 (Warrenfeltz et al. Gene expression profiling of epithelial ovarian tumours correlated with malignant potential: Molecular Cancer 2004, 3:27). It is a putative marker for prognosis of pancreatic cancer (Regine Brandt, et al.: DNA Microarray Analysis of Pancreatic Malignancies. Pancreatology 2004; 4:587-597; and also Edoardo Missiaglia, et al.: Analysis of gene expression in cancer cell lines identifies candidate markers for pancreatic tumorigenesis and metastasis. International Journal of Cancer (2004) Volume 112, Issue 1 Pages 100-112).

Table 168 below describes diagnostic utilities for the cluster T94936 that were found through microarrays, including the statistical significance thereof and a reference. One or more T94936 variants according to the present invention may optionally have one or more of these utilities.

TABLE 168

| | Statistical | |
|---|---|---|
| Diagnostic utility | significance | reference |
| Gene over expressed in Sporadic breast cancer (vs | 3E−4 | Van't Veer LJ, Friend SH |
| BRCA1 and BRCA2 mutated cancers); indicator | | Nature (2002) Gene expression |
| for hereditary breast cancers. | | profiling predicts clinical |
| | | outcome of breast cancer. |
| Gene over expressed in grade 1 breast cancer (vs. | 6E−7 | Van't Veer LJ, Friend SH |
| grade 3) | | Nature (2002) Gene expression |
| | | profiling predicts clinical |
| | | outcome of breast cancer. |
| Gene over expressed in negative Lymphocytic | 5.5E−7 | Van't Veer LJ, Friend SH |
| Infiltrate (vs. positive); negative indicator for | | Nature (2002) Gene expression |
| metastasis. | | profiling predicts clinical |
| | | outcome of breast cancer. |

Other non-limiting exemplary utilities for T94936 variants according to the present invention are described in greater detail below and also with regard to the previous section on clinical utility.

As noted above, contig T94936 features 3 transcript(s), which were listed in Table 164 above. A description of each variant protein according to the present invention is now provided.

Variant protein T94936_PEA_{—}1_PEA_{—}1_P2 (SEQ ID NO:427) according to the present invention has an amino acid sequence; it is encoded by transcript(s) T94936_PEA_{—}1_PEA_{—}1_T1 (SEQ ID NO:411). One or more alignments to one or more previously published protein sequences in the alignment table located on the attached CDROM. A brief description of the relationship of the variant protein according to the present invention to each such aligned protein is as follows:

Comparison report between T94936_PEA_{—}1_PEA_{—}1_P2 (SEQ ID NO:427) and Q8TD06 (SEQ ID NO:695) (SEQ ID NO:426):

1. An isolated chimeric polypeptide encoding for T94936_PEA_{—}1_PEA_{—}1_P2 (SEQ ID NO:427), comprising a first amino acid sequence being at least 90% homologous to MMLHSALGLCLLLVTVSSNLAIAIKKEKRPPQTLSRGWGDDITWVQTYEEGLFYAQKSKKPLMVI HHLEDCQYSQALKKVFAQNEEIQEMAQNKFIMLNLMHETTDKNLSPDGQYVPRIMFVDPSLTVRA DIAGRYSNRLYTYEPRDLPL corresponding to amino acids 1-150 of Q8TD06 (SEQ ID NO:695) (SEQ ID NO:426), which also corresponds to amino acids 1-150 of T94936_PEA_{—}1_PEA_{—}1_P2 (SEQ ID NO:427).

The location of the variant protein was determined according to results from a number of different software programs and analyses, including analyses from SignalP and other specialized programs. The variant protein is believed to be located as follows with regard to the cell: secreted.

Variant protein T94936_PEA_{—}1_PEA_{—}1_P2 (SEQ ID NO:427) is encoded by the following transcript(s): T94936_PEA_{—}1_PEA_{—}1_T1 (SEQ ID NO:411). The coding portion of transcript T94936_PEA_{—}1_PEA_{—}1_T1 (SEQ ID NO:411) starts at position 76 and ends at position 525. The transcript also has the following SNPs as listed in Table 169 (given according to their position on the nucleotide sequence, with the alternative nucleic acid listed; the last column indicates whether the SNP is known or not; the presence of known SNPs in variant protein T94936_PEA_{—}1_PEA_{—}1_P2 (SEQ ID NO:427) sequence provides support for the deduced sequence of this variant protein according to the present invention).

TABLE 169
Nucleic acid SNPs

| SNP position on | | |
|---|---|---|
| nucleotide sequence | Alternative nucleic acid | Previously known SNP? |
| 158 | A -> G | No |
| 186 | A -> G | No |
| 385 | A -> G | No |

Variant protein T94936_PEA_{—}1_PEA_{—}1_P3 (SEQ ID NO:428) according to the present invention has an amino acid sequence; it is encoded by transcript(s) T94936_PEA_{—}1_PEA_{—}1_T2 (SEQ ID NO:412). One or more alignments to one or more previously published protein sequences are given at in the alignment table located on the attached CDROM. A brief description of the relationship of the variant protein according to the present invention to each such aligned protein is as follows:

Comparison report between T94936_PEA_{—}1_PEA_{—}1_P3 (SEQ ID NO:428) and Q8TD06 (SEQ ID NO:695) (SEQ ID NO:426):

1. An isolated chimeric polypeptide encoding for T94936_PEA_{—}1_PEA_{—}1_P3 (SEQ ID NO:428), comprising a first amino acid sequence being at least 90% homologous to MMLHSALGLCLLLVTVSSNLAIAIKKEKRPPQTLSRGWGDDITWVQTYEEGLFYAQKSKKPLMVI HHLEDCQYSQALKKVFAQNEEIQEMAQNKFIMLNLMHETTDKNLSPDGQYVPRIMFV corresponding to amino acids 1-122 of Q8TD06 (SEQ ID NO:695) (SEQ ID NO:426), which also corresponds to amino acids 1-122 of T94936_PEA_{—}1_PEA_{—}1_P3 (SEQ ID NO:428), and a second amino acid sequence being at least 70%, optionally at least 80%, preferably at least 85%, more preferably at least 90% and most preferably at least 95% homologous to a polypeptide having the sequence GMYVISFHQIYKISRNQHSCFYF (SEQ ID NO: 660) corresponding to amino acids 123-145 of T94936_PEA_{—}1_PEA_{—}1_P3 (SEQ ID NO:428), wherein said first amino acid sequence and second amino acid sequence are contiguous and in a sequential order.

2. An isolated polypeptide encoding for a tail of T94936_PEA_{—}1_PEA_{—}1_P3 (SEQ ID NO:428), comprising a polypeptide being at least 70%, optionally at least about 80%, preferably at least about 85%, more preferably at least about 90% and most preferably at least about 95% homologous to the sequence GMYVISFHQIYKISRNQHSCFYF (SEQ ID NO: 660) in T94936_PEA_{—}1_PEA_{—}1_P3 (SEQ ID NO:428).

The location of the variant protein was determined according to results from a number of different software programs and analyses, including analyses from SignalP and other specialized programs. The variant protein is believed to be located as follows with regard to the cell: secreted.

Variant protein T94936_PEA_{—}1_PEA_{—}1_P3 (SEQ ID NO:428) is encoded by the following transcript(s): T94936_PEA_{—}1_PEA_{—}1T2 (SEQ ID NO:412). The coding portion of transcript T94936_PEA_{—}1_PEA_{—}1_T2 (SEQ ID NO:412) starts at position 76 and ends at position 510. The transcript also has the following SNPs as listed in Table 170 (given according to their position on the nucleotide sequence, with the alternative nucleic acid listed; the last column indicates whether the SNP is known or not; the presence of known SNPs in variant protein T94936_PEA_{—}1_PEA_{—}1_P3 (SEQ ID NO:428) sequence provides support for the deduced sequence of this variant protein according to the present invention).

TABLE 170
Nucleic acid SNPs

| SNP position on | | |
|---|---|---|
| nucleotide sequence | Alternative nucleic acid | Previously known SNP? |
| 158 | A -> G | No |
| 186 | A -> G | No |
| 385 | A -> G | No |
| 746 | T -> C | No |
| 889 | A -> C | No |
| 889 | A -> G | No |
| 980 | A -> | No |
| 1006 | A -> | No |
| 1105 | A -> | No |
| 1356 | A -> G | No |

Variant protein T94936_PEA_{—}1_PEA_{—}1_P7 (SEQ ID NO:429) according to the present invention has an amino acid sequence; it is encoded by transcript(s) T94936_PEA_{—}1_PEA_{—}1_T5 (SEQ ID NO:413). One or more alignments to one or more previously published protein sequences are given in the alignment table located on the attached CDROM. A brief description of the relationship of the variant protein according to the present invention to each such aligned protein is as follows:

Comparison report between T94936_PEA_{—}1_PEA_{—}1_P7 (SEQ ID NO:429) and Q8TD06 (SEQ ID NO:695) (SEQ ID NO:426):

1. An isolated chimeric polypeptide encoding for T94936_PEA_{—}1_PEA_{—}1_P7 (SEQ ID NO:429), comprising a first amino acid sequence being at least 90% homologous to MMLHSALGLCLLLVTVSSNLAIAIKKEKRPPQTLSRGWGDDITWVQTYEEGLFYAQK corresponding to amino acids 1-57 of Q8TD06 (SEQ ID NO:695) (SEQ ID NO:426), which also corresponds to amino acids 1-57 of T94936_PEA_{—}1_PEA_{—}1_P7 (SEQ ID NO:429), and a second amino acid sequence being at least 70%, optionally at least 80%, preferably at least 85%, more preferably at least 90% and most preferably at least 95% homologous to a polypeptide having the sequence RSH corresponding to amino acids 58-60 of T94936_PEA_{—}1_PEA_{—}1_P7 (SEQ ID NO:429), wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted.

Variant protein T94936_PEA_{—}1_PEA_{—}1_P7 (SEQ ID NO:429) is encoded by the following transcript(s): T94936_PEA_{—}1_PEA_{—}1_T5 (SEQ ID NO:413). The coding portion of transcript T94936_PEA_{—}1_PEA_{—}1_T5 (SEQ ID NO:413) starts at position 76 and ends at position 255. The transcript also has the following SNPs as listed in Table 171 (given according to their position on the nucleotide sequence, with the alternative nucleic acid listed; the last column indicates whether the SNP is known or not; the presence of known SNPs in variant protein T94936_PEA_{—}1_PEA_{—}1_P7 (SEQ ID NO:429) sequence provides support for the deduced sequence of this variant protein according to the present invention).

TABLE 171
Nucleic acid SNPs

| SNP position on | | |
|---|---|---|
| nucleotide sequence | Alternative nucleic acid | Previously known SNP? |
| 158 | A -> G | No |
| 186 | A -> G | No |
| 381 | A -> G | No |
| 581 | A -> G | No |

Table 172 below describes the starting and ending position of T94936_PEA_{—}1_PEA_{—}1_node_{—}14 (SEQ ID NO:415) on the relevant transcript. Experimental results for this segment are described below.

TABLE 172
Segment location on transcripts

| | Segment | |
|---|---|---|
| | starting | Segment |
| Transcript name | position | ending position |
| T94936_PEA_1_PEA_1_T2 (SEQ ID | 443 | 803 |
| NO: 412) | | |

Table 173 below describes the starting and ending position of T94936_PEA_{—}1_PEA_{—}1_node_{—}20 (SEQ ID NO:417) on each of the relevant transcripts. Experimental results for this segment are described below.

TABLE 173
Segment location on transcripts

| | Segment | |
|---|---|---|
| | starting | Segment |
| Transcript name | position | ending position |
| T94936_PEA_1_PEA_1_T2 (SEQ ID | 1298 | 1526 |
| NO: 412) | | |
| T94936_PEA_1_PEA_1_T5 (SEQ ID | 523 | 751 |
| NO: 413) | | |

Expression of Homo sapiens breast cancer membrane protein 11 (BCMP11) T94936 transcripts which are detectable by amplicon as depicted in sequence name T94936 seg14 (SEQ ID NO: 563) in different normal tissues

Expression of Homo sapiens breast cancer membrane protein 11 (BCMP11) transcripts detectable by or according to T94936 seg14 (SEQ ID NO: 563) amplicon and primers: T94936 seg14F (SEQ ID NO:561) and T94936 seg14R (SEQ ID NO:562) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the breast samples (Sample Nos. 33-35 Table 7, “Tissue samples in normal panel”), to obtain a value of relative expression of each sample relative to median of the breast samples.

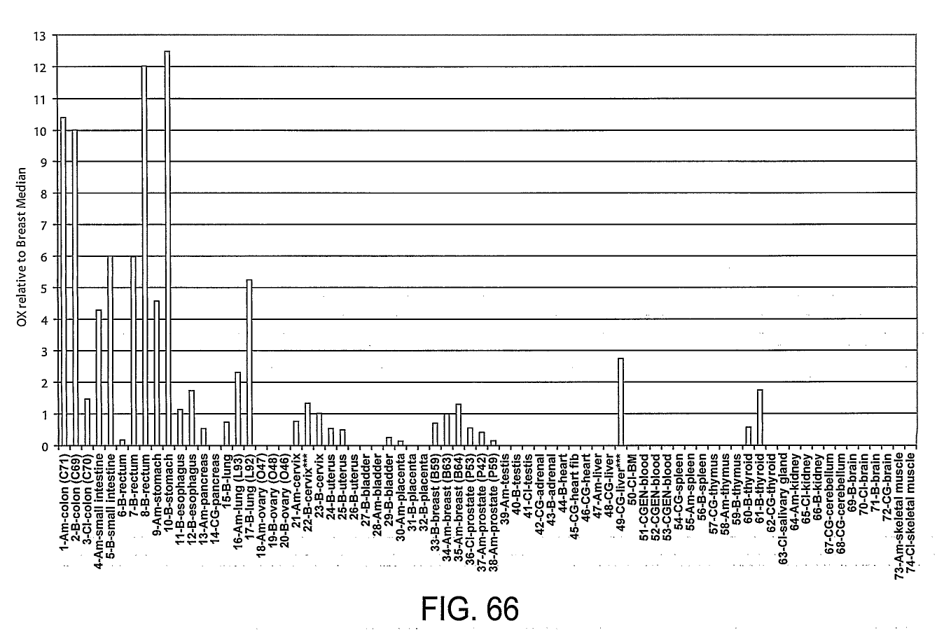

FIG. 66 is a histogram showing expression of Homo sapiens breast cancer membrane protein 11 (BCMP11) T94936 transcripts which are detectable by amplicon as depicted in sequence name T94936 seg14 (SEQ ID NO: 563) in different normal tissues

Primers:

Forward primer T94936 seg14F (SEQ ID NO:561):
TACAAAATTAGTAGAAATCAGCATTCTTGC
Reverse primer T94936 seg14R (SEQ ID NO:562):
TGTAGAACTAACAAGAGCTGATATTATTGGAT
Amplicon T94936 seg14 (SEQ ID NO:563):
TACAAAATTAGTAGAAATCAGCATTCTTGCTTTTATTTTTAAATGCTAGT
TCAAGTACTATTCTTTTTAAAGAGAAGTCATTTCTAATCCAATAATATCA
GCTCTTGTTAGTTCTACA

Expression of Homo sapiens breast cancer membrane protein 11 (BCMP11) T94936 transcripts which are detectable by amplicon as depicted in sequence name T94936 seg14 (SEQ ID NO: 563) in normal and cancerous colon tissues

Expression of Homo sapiens breast cancer membrane protein 11 (BCMP11) transcripts detectable by or according to seg14, T94936 seg14 (SEQ ID NO: 563) amplicon and primers T94936 seg14F (SEQ ID NO:561) and T94936 seg14R (SEQ ID NO:562) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), G6PD (GenBank Accession No. NM_{—}000402 (SEQ ID NO:8); G6PD amplicon (SEQ ID NO: 44)), RPS27A (GenBank Accession No. NM_{—}002954 (SEQ ID NO:1); RPS27A amplicon (SEQ ID NO: 47)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 4, above, “Tissue samples in colon cancer testing panel”), to obtain a value of fold differential expression for each sample relative to median of the normal PM samples.

In one experiment that was carried out no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: T94936 seg14F (SEQ ID NO:561) forward primer; and T94936 seg14R (SEQ ID NO:562) reverse primer.

The present invention also preferably encompasses any amplicon obtained through the use of any suitable primer pair; for example, for the above experiment, the following amplicon was obtained as a non-limiting illustrative example only of a suitable amplicon: T94936 seg14 (SEQ ID NO: 563).

Primers:

Forward primer T94936 seg14F (SEQ ID NO:561):
TACAAAATTAGTAGAAATCAGCATTCTTGC
Reverse primer T94936 seg14R (SEQ ID NO:562):
TGTAGAACTAACAAGAGCTGATATTATTGGAT
Amplicon T94936 seg14 (SEQ ID NO:563):
TACAAAATTAGTAGAAATCAGCATTCTTGCTTTTATTTTTAAATGCTAGT
TCAAGTACTATTCTTTTTAAAGAGAAGTCATTTCTAATCCAATAATATCA
GCTCTTGTTAGTTCTACA

Expression of Homo sapiens breast cancer membrane protein 11 (BCMP11) T94936 transcripts which are detectable by amplicon as depicted in sequence name T94936 seg14 (SEQ ID NO: 563) in normal and cancerous lung tissues

Expression of Homo sapiens breast cancer membrane protein 11 (BCMP11) transcripts detectable by or according to seg14, T94936 seg14 (SEQ ID NO: 563) amplicon and primers T94936 seg14F (SEQ ID NO:561) and T94936 seg14R (SEQ ID NO:562) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 47-50, 90-93, 96-99, Table 5, above, “Tissue samples in lung cancer testing panel”), to obtain a value of fold differential expression for each sample relative to median of the normal PM samples.

In one experiment that was carried out no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: T94936 seg14F (SEQ ID NO:561) forward primer; and T94936 seg14R (SEQ ID NO:562) reverse primer.

The present invention also preferably encompasses any amplicon obtained through the use of any suitable primer pair; for example, for the above experiment, the following amplicon was obtained as a non-limiting illustrative example only of a suitable amplicon: T94936 seg14 (SEQ ID NO: 563).

Primers:

Forward primer T94936 seg14F (SEQ ID NO:561):
TACAAAATTAGTAGAAATCAGCATTCTTGC
Reverse primer T94936 seg14R (SEQ ID NO:562):
TGTAGAACTAACAAGAGCTGATATTATTGGAT
Amplicon T94936 seg14 (SEQ ID NO:563):
TACAAAATTAGTAGAAATCAGCATTCTTGCTTTTATTTTTAAATGCTAGT
TCAAGTACTATTCTTTTTAAAGAGAAGTCATTTCTAATCCAATAATATCA
GCTCTTGTTAGTTCTACA

Expression of Homo sapiens breast cancer membrane protein 11 (BCMP11) T94936 transcripts which are detectable by amplicon as depicted in sequence name T94936 seg14 (SEQ ID NO: 563) in normal and cancerous breast tissues

Expression of Homo sapiens breast cancer membrane protein 11 (BCMP11) transcripts detectable by or according to seg14, T94936 seg14 (SEQ ID NO: 563) amplicon and primers T94936 seg14F (SEQ ID NO:561) and T94936 seg14R (SEQ ID NO:562) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), G6PD (GenBank Accession No. NM_{—}000402 (SEQ ID NO:8); G6PD amplicon (SEQ ID NO: 44)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67, Table 6, above, “Tissue samples in breast cancer testing panel”), to obtain a value of fold up-regulation for each sample relative to median of the normal PM samples.

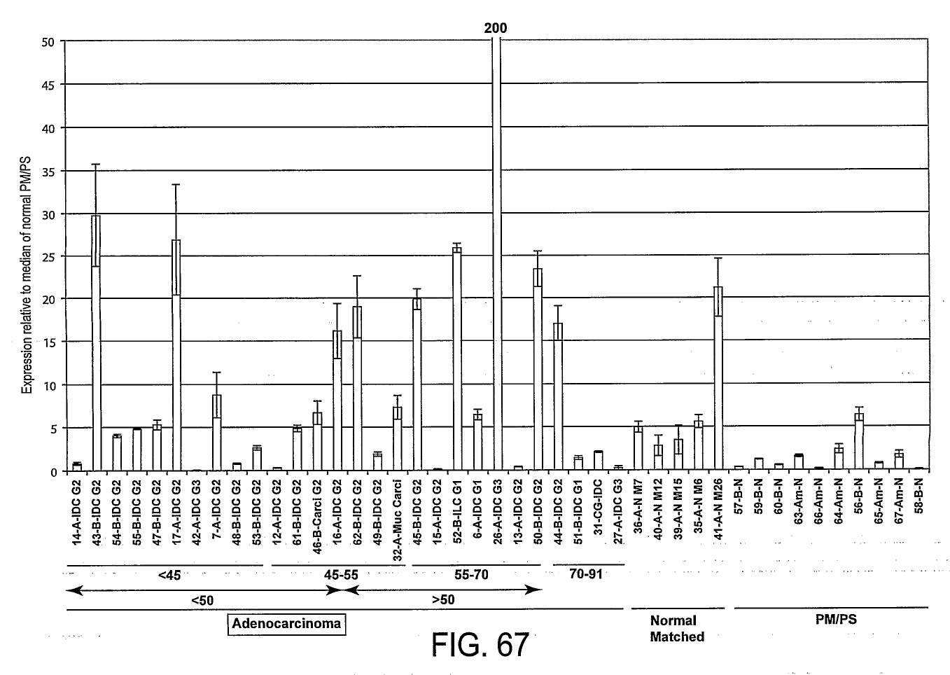

FIG. 67 is a histogram showing over expression of the above-indicated Homo sapiens breast cancer membrane protein 11 (BCMP11) transcripts in cancerous breast samples relative to the normal samples. Values represent the average of duplicate experiments. Error bars indicate the minimal and maximal values obtained.

As is evident from FIG. 67, the expression of Homo sapiens breast cancer membrane protein 11 (BCMP11) transcripts detectable by the above amplicon in cancer samples was significantly higher than in the non-cancerous samples (Sample Nos. 56-60, 63-67 Table 6, “Tissue samples in breast cancer testing panel”). Notably an over-expression of at least 5 fold was found in 16 out of 28 adenocarcinoma samples.

Statistical analysis was applied to verify the significance of these results, as described below.

The P value for the difference in the expression levels of Homo sapiens breast cancer membrane protein 11 (BCMP11) transcripts detectable by the above amplicon in breast cancer samples versus the normal tissue samples was determined by T test as 5.76E-02.

Threshold of 5 fold overexpression was found to differentiate between cancer and normal samples with P value of 1.13E-02 as checked by exact fisher test. The above values demonstrate statistical significance of the results.

By dissecting the patient's population into two age groups, indicated below the graph, we demonstrate that the transcripts detected by the above amplicon are highly expressed mainly in patients with an age above 50

Primer pairs are also optionally and preferably encompassed within the present invention; for example, for the above experiment, the following primer pair was used as a non-limiting illustrative example only of a suitable primer pair: T94936 seg14F (SEQ ID NO:561) forward primer; and T94936 seg14R (SEQ ID NO:562) reverse primer.

The present invention also preferably encompasses any amplicon obtained through the use of any suitable primer pair; for example, for the above experiment, the following amplicon was obtained as a non-limiting illustrative example only of a suitable amplicon: T94936 seg14 (SEQ ID NO: 563).

Primers:

Forward primer T94936 seg14F (SEQ ID NO:561):
TACAAAATTAGTAGAAATCAGCATTCTTGC
Reverse primer T94936 seg14R (SEQ ID NO:562):
TGTAGAACTAACAAGAGCTGATATTATTGGAT
Amplicon T94936 seg14 (SEQ ID NO:563):
TACAAAATTAGTAGAAATCAGCATTCTTGCTTTTATTTTTAAATGCTAGT
TCAAGTACTATTCTTTTTAAAGAGAAGTCATTTCTAATCCAATAATATCA
GCTCTTGTTAGTTCTACA

Expression of Homo sapiens breast cancer membrane protein 11 (BCMP11) T94936 transcripts which are detectable by amplicon as depicted in sequence name T94936 seg14 (SEQ ID NO: 563) in normal and cancerous ovary tissues

Expression of Homo sapiens breast cancer membrane protein 11 (BCMP11) transcripts detectable by or according to seg14, T94936 seg14 (SEQ ID NO: 563) amplicon and primers T94936 seg14F (SEQ ID NO:561) and T94936 seg14R (SEQ ID NO:562) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and GAPDH (GenBank Accession No. BC026907 (SEQ ID NO:3); GAPDH amplicon (SEQ ID NO: 41) was measured similarly. For each RT sample, the expression of the above amplicon was normalized to the geometric mean of the quantities of the housekeeping genes. The normalized quantity of each RT sample was then divided by the average of the quantities of the normal post-mortem (PM) samples (Sample Nos. 45, 46, 48, 71, Table 3, above, “Tissue samples in ovarian cancer testing panel”), to obtain a value of fold up-regulation for each sample relative to median of the normal PM samples.

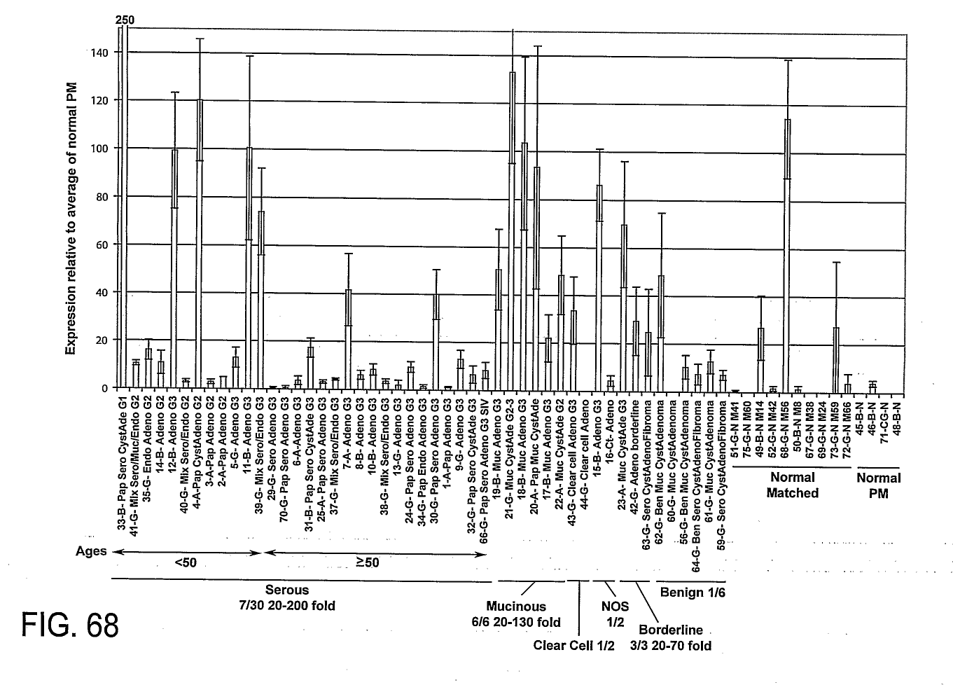

FIG. 68 is a histogram showing over expression of the above-indicated Homo sapiens breast cancer membrane protein 11 (BCMP11) transcripts in cancerous ovary samples relative to the normal samples. Values represent the average of duplicate experiments. Error bars indicate the minimal and maximal values obtained.

As is evident from FIG. 68, the expression of Homo sapiens breast cancer membrane protein 11 (BCMP11) transcripts detectable by the above amplicon in cancer samples was significantly higher than in the non-cancerous samples (Sample Nos. 45, 46, 48, 71 Table 3, “Tissue samples in ovarian cancer testing panel”). Notably an over-expression of at least 10 fold was found in 13 out of 30 serous adenocarcinoma samples and 6 out of 6 mucinous cancer samples, 1 out of the 2 clear cell samples and 3 out of the 3 borderline samples.

Statistical analysis was applied to verify the significance of these results, as described below.

The P value for the difference in the expression levels of Homo sapiens breast cancer membrane protein 11 (BCMP11) transcripts detectable by the above amplicon in ovary serous cancer samples versus the normal tissue samples was determined by T test as 6.90E-03.

The P value for the difference in the expression levels of Homo sapiens breast cancer membrane protein 11 (BCMP11) transcripts detectable by the above amplicon in ovary mucinous cancer samples versus the normal tissue samples was determined by T test as 7.18E-03.

The above values demonstrate statistical significance of the results.

These results also demonstrate the transcripts detected by the above amplicon are highly expressed mainly in Mucinous ovarian cancer. Also, patients having serous adenocarcinoma with an age below 50 show higher expression of this variant.

Primer pairs are also optionally and preferably encompassed within the present invention; for example, for the above experiment, the following primer pair was used as a non-limiting illustrative example only of a suitable primer pair: T94936 seg14F (SEQ ID NO:561) forward primer; and T94936 seg14R (SEQ ID NO:562) reverse primer.

The present invention also preferably encompasses any amplicon obtained through the use of any suitable primer pair; for example, for the above experiment, the following amplicon was obtained as a non-limiting illustrative example only of a suitable amplicon: T94936 seg14 (SEQ ID NO: 563).

Primers:

Forward primer T94936 seg14F (SEQ ID NO:561):
TACAAAATTAGTAGAAATCAGCATTCTTGC
Reverse primer T94936 seg14R (SEQ ID NO:562):
TGTAGAACTAACAAGAGCTGATATTATTGGAT
Amplicon T94936 seg14 (SEQ ID NO:563):
TACAAAATTAGTAGAAATCAGCATTCTTGCTTTTATTTTTAAATGCTAGT
TCAAGTACTATTCTTTTTAAAGAGAAGTCATTTCTAATCCAATAATATCA
GCTCTTGTTAGTTCTACA

Expression of Homo sapiens breast cancer membrane protein 11 (BCMP11) T94936 transcripts which are detectable by amplicon as depicted in sequence name T94936 seg20 (SEQ ID NO: 432) in normal and cancerous ovary tissues

Expression of Homo sapiens breast cancer membrane protein 11 (BCMP11) transcripts detectable by or according to seg20-T94936 seg20 (SEQ ID NO: 432) amplicon and primers T94936 seg20F (SEQ ID NO: 430) and T94936 seg20R (SEQ ID NO: 431) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold up-regulation for each sample relative to median of the normal PM samples.

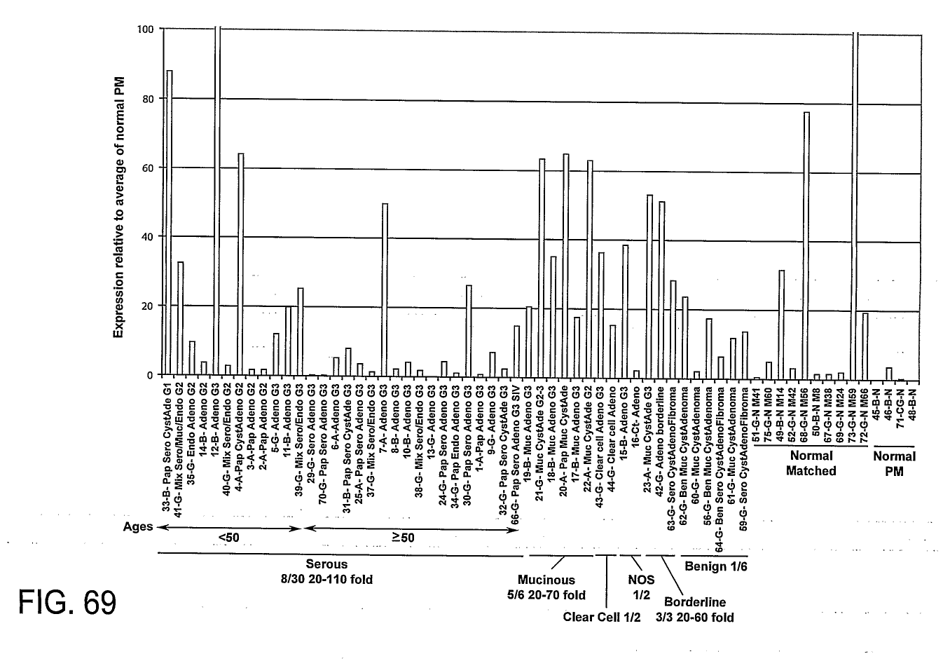

FIG. 69 is a histogram showing over expression of the above-indicated Homo sapiens breast cancer membrane protein 11 (BCMP11) transcripts in cancerous ovary samples relative to the normal samples.

As is evident from FIG. 69, the expression of Homo sapiens breast cancer membrane protein 11 (BCMP11) transcripts detectable by the above amplicon in cancer samples was significantly higher than in the non-cancerous samples (Sample Nos. 45, 46, 48, 71 Table 3, “Tissue samples in ovarian cancer testing panel”). Notably an over-expression of at least 10 fold was found in 10 out of 30 serous adenocarcinoma samples and 6 out of 6 mucinous adenocarcinoma samples, 1 out of the 2 clear cell samples and 3 out of the 3 borderline samples.

Statistical analysis was applied to verify the significance of these results, as described below.

The P value for the difference in the expression levels of Homo sapiens breast cancer membrane protein 11 (BCMP11) transcripts detectable by the above amplicon(s) in ovary serous cancer samples versus the normal tissue samples was determined by T test as 3.84E-03.

The P value for the difference in the expression levels of Homo sapiens breast cancer membrane protein 11 (BCMP11) transcripts detectable by the above amplicon(s) in ovary mucinous cancer samples versus the normal tissue samples was determined by T test as 5.44E-03. The above values demonstrate statistical significance of the results.

These results demonstrate the transcripts detected by the above amplicon are highly expressed mainly in Mucinous ovarian cancer. Also, patients having serous adenocarcinoma with an age below 50 show higher expression of this variant

Primer pairs are also optionally and preferably encompassed within the present invention; for example, for the above experiment, the following primer pair was used as a non-limiting illustrative example only of a suitable primer pair: T94936 seg20F (SEQ ID NO: 430) forward primer; and T94936 seg20R (SEQ ID NO: 431) reverse primer.

The present invention also preferably encompasses any amplicon obtained through the use of any suitable primer pair; for example, for the above experiment, the following amplicon was obtained as a non-limiting illustrative example only of a suitable amplicon: T94936 seg20 (SEQ ID NO: 432).

Primers:

Forward primer T94936 seg20F (SEQ ID NO:430):
AAAGCATTAAGACTTATTCAGTCAGAGC
Reverse primer T94936 seg20R (SEQ ID NO:431):
TCAATGTGCCAGAGGTTTTCTTC
Amplicon T94936 seg20 (SEQ ID NO:432):
AAAGCATTAAGACTTATTCAGTCAGAGCTATAAGAGATGATAGAAAAAAG
CCTTCACTTCAAAGAAGTCAAATTTCATGAAGAAAACCTCTGGCACATTG
A

Expression of Homo sapiens breast cancer membrane protein 11 (BCMP11) T94936 transcripts which are detectable by amplicon as depicted in sequence name T94936 seg20 (SEQ ID NO: 432) in normal and cancerous breast tissues

Expression of Homo sapiens breast cancer membrane protein 11 (BCMP11) transcripts detectable by or according to seg20-T94936 seg20 (SEQ ID NO: 432) amplicon and primers T94936 seg20F (SEQ ID NO: 430) and T94936 seg20R (SEQ ID NO: 431) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), G6PD (GenBank Accession No. NM_{—}000402 (SEQ ID NO:8); G6PD amplicon (SEQ ID NO: 44)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 56-60, 63-67, Table 6, above, “Tissue samples in breast cancer testing panel”), to obtain a value of fold up-regulation for each sample relative to median of the normal PM samples.

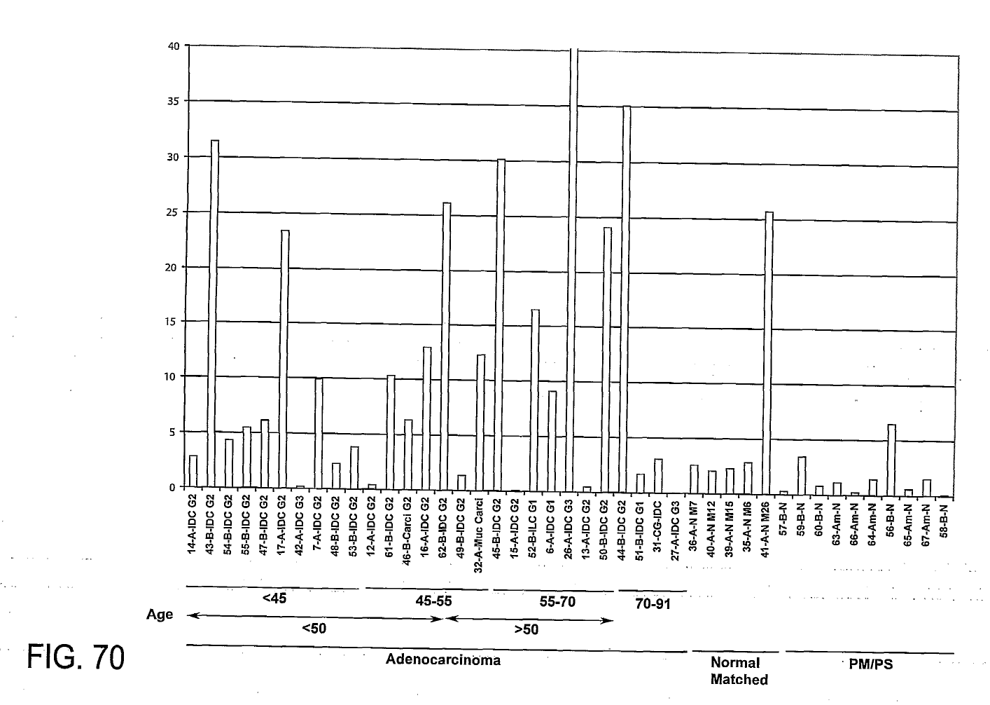

FIG. 70 is a histogram showing over expression of the above-indicated Homo sapiens breast cancer membrane protein 11 (BCMP11) transcripts in cancerous breast samples relative to the normal samples.

As is evident from FIG. 70, the expression of Homo sapiens breast cancer membrane protein 11 (BCMP11) transcripts detectable by the above amplicon in cancer samples was significantly higher than in the non-cancerous samples (Sample Nos. 56-60, 63-67 Table 6, “Tissue samples in breast cancer testing panel”). Notably an over-expression of at least 5 fold was found in 16 out of 28 adenocarcinoma samples.

Statistical analysis was applied to verify the significance of these results, as described below.

The P value for the difference in the expression levels of Homo sapiens breast cancer membrane protein 11 (BCMP11) transcripts detectable by the above amplicon in breast cancer samples versus the normal tissue samples was determined by T test as 8.64E-03.

Threshold of 5 fold overexpression was found to differentiate between cancer and normal samples with P value of 1.13E-02 as checked by exact fisher test. The above values demonstrate statistical significance of the results.

By dissecting the patient's population into two age groups (indicated below the graph), we demonstrate that the above amplicon is highly expressed mainly in patients with an age above 50.

Primer pairs are also optionally and preferably encompassed within the present invention; for example, for the above experiment, the following primer pair was used as a non-limiting illustrative example only of a suitable primer pair: T94936 seg20F (SEQ ID NO: 430) forward primer; and T94936 seg20R (SEQ ID NO: 431) reverse primer.

The present invention also preferably encompasses any amplicon obtained through the use of any suitable primer pair; for example, for the above experiment, the following amplicon was obtained as a non-limiting illustrative example only of a suitable amplicon: T94936 seg20 (SEQ ID NO: 432).

Primers:

Forward primer T94936 seg20F (SEQ ID NO:430):
AAAGCATTAAGACTTATTCAGTCAGAGC
Reverse primer T94936 seg20R (SEQ ID NO:431):
TCAATGTGCCAGAGGTTTTCTTC
Amplicon T94936 seg20 (SEQ ID NO:432):
AAAGCATTAAGACTTATTCAGTCAGAGCTATAAGAGATGATAGAAAAAAG
CCTTCACTTCAAAGAAGTCAAATTTCATGAAGAAAACCTCTGGCACATTG
A

Expression of Homo sapiens breast cancer membrane protein 11 (BCMP11) T94936 transcripts which are detectable by amplicon as depicted in sequence name T94936 seg20 (SEQ ID NO: 432) in different normal tissues

Expression of Homo sapiens breast cancer membrane protein 11 (BCMP11) transcripts detectable by or according to T94936 seg20 (SEQ ID NO: 432) amplicon and primers: T94936 seg20F (SEQ ID NO: 430) and T94936 seg20R (SEQ ID NO: 431) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. The normalized quantity of each RT sample was then divided by the median of the quantities of the breast samples (Sample Nos. 33-35 Table 7, “Tissue samples in normal panel”), to obtain a value of relative expression of each sample relative to median of the breast samples.

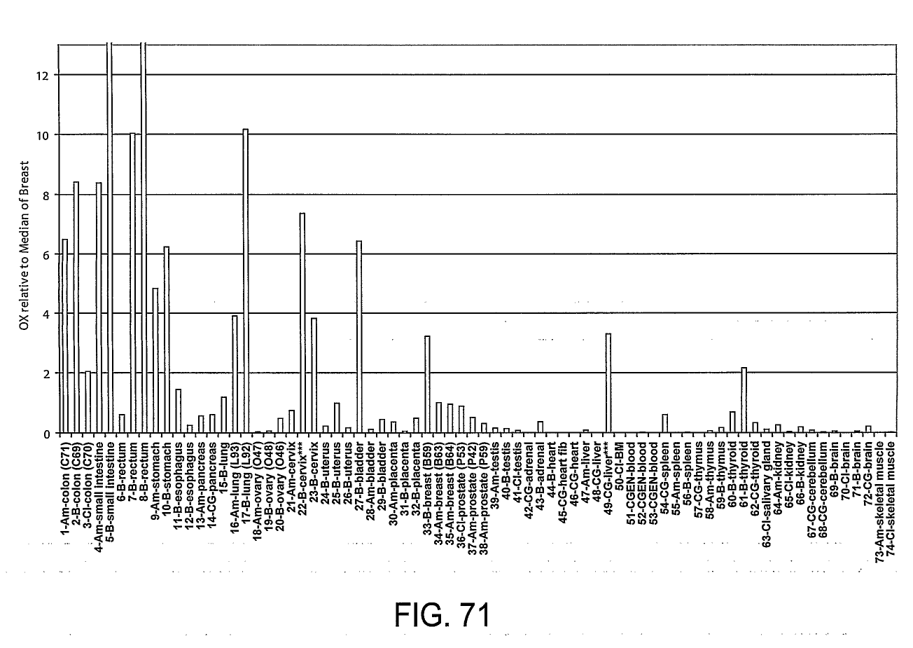

FIG. 71 is a histogram showing Expression of Homo sapiens breast cancer membrane protein 11 (BCMP11) T94936 transcripts which are detectable by amplicon as depicted in sequence name T94936 seg20 (SEQ ID NO: 432) in different normal tissues

Primers:

Forward primer T94936 seg20F (SEQ ID NO:430):
AAAGCATTAAGACTTATTCAGTCAGAGC
Reverse primer T94936 seg20R (SEQ ID NO:431):
TCAATGTGCCAGAGGTTTTCTTC
Amplicon T94936 seg20 (SEQ ID NO:432):
AAAGCATTAAGACTTATTCAGTCAGAGCTATAAGAGATGATAGAAAAAAG
CCTTCACTTCAAAGAAGTCAAATTTCATGAAGAAAACCTCTGGCACATTG
A

### Description for Cluster HSTGFB1

Cluster HSTGFB1 features 6 transcript(s) and 24 segment(s) of interest, the names for which are given in Tables 174 and 175. The selected protein variants are given in table 176.

TABLE 174
Transcripts of interest
Transcript Name

HSTGFB1_T5 (SEQ ID NO: 433)
HSTGFB1_T6 (SEQ ID NO: 434)
HSTGFB1_T8 (SEQ ID NO: 435)
HSTGFB1_T9 (SEQ ID NO: 436)
HSTGFB1_T11 (SEQ ID NO: 437)
HSTGFB1_T14 (SEQ ID NO: 438)

TABLE 175
Segments of interest

Segment Name

| | |
|---|---|
| HSTGFB1_node_0 (SEQ ID NO: 439) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_2 (SEQ ID NO: 440) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_3 (SEQ ID NO: 441) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_4 (SEQ ID NO: 442) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_7 (SEQ ID NO: 443) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_9 (SEQ ID NO: 444) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_15 (SEQ ID NO: 445) | HSTGFB1_T5 (SEQ ID NO: 433) |
| HSTGFB1_node_22 (SEQ ID NO: 446) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ lD NO: 438) |
| HSTGFB1_node_26 (SEQ ID NO: 447) | HSTGFB1_T5 (SEQ ID NO: 433), |
| | HSTGFB1_T6 (SEQ ID NO: 434), |
| | HSTGFB1_T11 (SEQ ID NO: 437) and |
| | HSTGFB1_T14 (SEQ ID NO: 438) |
| HSTGFB1_node_28 (SEQ ID NO: 448) | HSTGFB1_T5 (SEQ ID NO: 433), |
| | HSTGFB1_T6 (SEQ ID NO: 434), |
| | HSTGFB1_T11 (SEQ ID NO: 437) and |
| | HSTGFB1_T14 (SEQ ID NO: 438) |
| HSTGFB1_node_31 (SEQ ID NO: 449) | HSTGFB1_T9 (SEQ ID NO: 436) |
| HSTGFB1_node_33 (SEQ ID NO: 450) | HSTGFB1_T8 (SEQ ID NO: 435) |
| HSTGFB1_node_1 (SEQ ID NO: 451) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_5 (SEQ ID NO: 452) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_11 (SEQ ID NO: 453) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_14 (SEQ ID NO: 454) | HSTGFB1_T5 (SEQ ID NO: 433), |
| | HSTGFB1_T6 (SEQ ID NO: 434), |
| | HSTGFB1_T8 (SEQ ID NO: 435), |
| | HSTGFB1_T9 (SEQ ID NO: 436), |
| | HSTGFB1_T11 (SEQ ID NO: 437) and |
| | HSTGFB1_T14 (SEQ ID NO: 438) |
| HSTGFB1_node_16 (SEQ ID NO: 455) | HSTGFB1_T5 (SEQ ID NO: 433), |
| | HSTGFB1_T6 (SEQ ID NO: 434), |
| | HSTGFB1_T8 (SEQ ID NO: 435) and |
| | HSTGFB1_T9 (SEQ ID NO: 436) |
| HSTGFB1_node_17 (SEQ ID NO: 456) | HSTGFB1_T5 (SEQ ID NO: 433), |
| | HSTGFB1_T6 (SEQ ID NO: 434), |
| | HSTGFB1_T8 (SEQ ID NO: 435) and |
| | HSTGFB1_T9 (SEQ ID NO: 436) |
| HSTGFB1_node_18 (SEQ ID NO: 457) | HSTGFB1_T5 (SEQ ID NO: 433), |
| | HSTGFB1_T6 (SEQ ID NO: 434), |
| | HSTGFB1_T8 (SEQ ID NO: 435) and |
| | HSTGFB1_T9 (SEQ ID NO: 436) |
| HSTGFB1_node_19 (SEQ ID NO: 458) | HSTGFB1_T5 (SEQ ID NO: 433), |
| | HSTGFB1_T6 (SEQ ID NO: 434), |
| | HSTGFB1_T8 (SEQ ID NO: 435) and |
| | HSTGFB1_T9 (SEQ ID NO: 436) |
| HSTGFB1_node_23 (SEQ ID NO: 459) | HSTGFB1_T6 (SEQ ID NO: 434) and |
| | HSTGFB1_T14 (SEQ ID NO: 438) |
| HSTGFB1_node_25 (SEQ ID NO: 460) | HSTGFB1_T5 (SEQ ID NO: 433), |
| | HSTGFB1_T6 (SEQ ID NO: 434), |
| | HSTGFB1_T11 (SEQ ID NO: 437) and |
| | HSTGFB1_T14 (SEQ ID NO: 438) |
| HSTGFB1_node_27 (SEQ ID NO: 461) | HSTGFB1_T5 (SEQ ID NO: 433), |
| | HSTGFB1_T6 (SEQ ID NO: 434), |
| | HSTGFB1_T11 (SEQ ID NO: 437) and |
| | HSTGFB1_T14 (SEQ ID NO: 438) |
| HSTGFB1_node_30 (SEQ ID NO: 462) | HSTGFB1_T8 (SEQ ID NO: 435) and |
| | HSTGFB1_T9 (SEQ ID NO: 436) |

TABLE 176
Proteins of interest

| Protein Name | Corresponding Transcript(s) |
|---|---|
| HSTGFB1_P2 (SEQ ID NO: 464) | HSTGFB1_T5 (SEQ ID NO: 433) |
| HSTGFB1_P3 (SEQ ID NO: 465) | HSTGFB1_T6 (SEQ ID NO: 434) |
| HSTGFB1_P5 (SEQ ID NO: 466) | HSTGFB1_T8 (SEQ ID NO: 435); |
| | HSTGFB1_T9 (SEQ ID NO: 436) |
| HSTGFB1_P7 (SEQ ID NO: 467) | HSTGFB1_T11 (SEQ ID NO: 437) |
| HSTGFB1_P10 (SEQ ID NO: | HSTGFB1_T14 (SEQ ID NO: 438) |
| 468) | |

These sequences are variants of the known protein Transforming growth factor beta 1 precursor (SEQ ID NO:463) (SwissProt accession identifier TGFB1_HUMAN (SEQ ID NO:463); known also according to the synonyms TGF-beta 1), referred to herein as the previously known protein.

Protein Transforming growth factor beta 1 precursor (SEQ ID NO:463) is known or believed to have the following function(s): Multifunctional peptide that controls proliferation, differentiation, and other functions in many cell types. Many cells synthesize TGF-beta 1 and essentially all of them have specific receptors for this peptide. TGF-beta 1 regulates the actions of many other peptide growth factors and determines a positive or negative direction of their effects. Play an important role in bone remodelling. It is a potent stimulator of osteoblastic bone formation, causing chemotaxis, proliferation and differentiation in committed osteoblasts (By similarity). Known polymorphisms for this sequence are as shown in Table 177.

TABLE 177
Amino acid mutations for Known Protein

| SNP | |
|---|---|
| position(s) | |
| on amino | |
| acid | |
| sequence | Comment |
| 10 | L -> P (associated with higher bone mineral density and lower |
| | frequency of vertebral fractures in Japanese post-menopausal |
| | women; dbSNP: 1982073). /FTId = VAR_016171. |
| 25 | R -> P (in dbSNP: 1800471). /FTId = VAR_016172. |
| 81 | Y -> H (in CED; leads to TGF-beta 1 intracellular |
| | accumulation)./FTId = VAR_017607. |
| 218 | R -> C (in CED; higher levels of active TGF-beta 1 in |
| | the culture medium; enhances osteoclast formation in |
| | vitro). /FTId = VAR_017608. |
| 218 | R -> H (in CED). /FTId = VAR_017609. |
| 222 | H -> D (in CED; sporadic case; higher levels of active |
| | TGF-beta 1 in the culture medium). /FTId = VAR_017610. |
| 225 | C -> R (in CED; higher levels of active TGF-beta 1 in |
| | the culture medium). /FTId = VAR_017611. |
| 263 | T -> I (in dbSNP: 1800472). /FTId = VAR_016173. |
| 159 | R -> RR |

Protein Transforming growth factor beta 1 precursor (SEQ ID NO:463) localization is believed to be Secreted.

It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Immunosuppressant; Interleukin 2 antagonist; Transforming growth factor beta agonist. A therapeutic role for a protein represented by the cluster has been predicted. The cluster was assigned this field because there was information in the drug database or the public databases (e.g., described herein above) that this protein, or part thereof, is used or can be used for a potential therapeutic indication: Vulnerary; Cytokine; Immunosuppressant.

The following GO Annotation(s) apply to the previously known protein. The following annotation(s) were found: cell cycle control; anti-apoptosis; TGFbeta receptor signaling pathway; cell-cell signaling; cell proliferation; cell growth; growth, which are annotation(s) related to Biological Process; and transforming growth factor beta receptor ligand, which are annotation(s) related to Molecular Function.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of HSTGFB1) may optionally have one or more of the following utilities, as described with regard to Table 178 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 178
Utilities for Variants of HSTGFB1

| | | |
|---|---|---|
| Late-onset Alzheimer's | significant increase of sCD40 plasma levels, | Exp Gerontol. 2004 |
| disease (AD) | concomitantly with a decrease in TGF-beta1 | Oct; 39(10): 1555-61 |
| | concentrations might represent possible | |
| | differential biomarkers of AD, and be useful pre- | |
| | mortem to support the clinical diagnosis of late- | |
| | onset AD. | |
| coronary perivascular | Markedly increased PAI-1 and TGF-beta were | J Mol Cell Cardiol. |
| fibrosis associated with | seen immunohistochemically in coronary vascular | 2004 Aug; 37(2): 525-35 |
| insulin resistance and | wall and confirmed by western blotting in obese | |
| obesity | mice. Treatment of these mice with temocapril | |
| | prevented increases in perivascular fibrosis, | |
| | plasma PAI-1 and TGF-beta(1), left ventricular | |
| | collagen and mural immunoreactivity for PAI-1, | |
| | TGF-beta and collagen type 1 | |
| ulcerative colitis (UC) | Using specific antibodies it was shown that IL-1ra | Inflamm Res. 2004 |
| | and TGF-beta 1 appear to modulate the degree of | Feb; 53(2): 53-9. Epub |
| | inflammation at different stages of the | 2004 Jan 26. |
| | inflammatory process. The levels of TGF-beta 1, | |
| | and its effect in controlling inflammation, was | |
| | most marked in mild but not severe UC | |
| breast cancer, diagnosis | Patients who carried the C allele of T + 29C | Cancer Res. 2004 Feb |
| and prognosis | polymorphism had a reduced 5-year disease-free | 1; 64(3): 836-9 |
| | survival rate. Genetic polymorphisms in the TGF- | |
| | beta1 gene may play a role in breast cancer | |
| | progression | |
| larynx squamous | Ten of the 21 specimens of the larynx squamous | Lin Chuang Er Bi Yan |
| carcinoma | carcinoma showed positive expression of TGF- | Hou Ke Za Zhi. 2003 |
| | beta 1 mRNA, then the expression ratio was | Sep; 17(9): 532-4 |
| | 47.62%. The expression of TGF-beta 1 mRNA had | |
| | relationship to the pathological grading and the | |
| | clinical staging of the larynx carcinoma. | |
| laryngeal carcinoma | The expression of TGF-beta 1 was decreased in | Lin Chuang Er Bi Yan |
| | carcinoma tissues when compared with peri-cancer | Hou Ke Za Zhi. 2003 |
| | controls. There was no difference in T beta R II | Jun; 17(6): 336-8 |
| | expression when compared with peri cancer | |
| | controls (P > 0.05). But it was correlated with the | |
| | stage, differentiation and lymph node metastasis of | |
| | laryngeal carcinoma | |
| advanced breast cancer | Plasma TGF-beta 1 values were significantly | Eur J Cancer. 2003 |
| | elevated (P < 0.05) in stage IIIB/IV patients | Mar; 39(4): 454-61 |
| | (median value: 2.40 ng/ml, range: 0.13-8.48 ng/ml, | |
| | n = 44) compared with healthy donors (median | |
| | value: 1.30 ng/ml, range: 0.41-4.93 ng/ml, n = 36). | |
| cirrhosis | TGF beta 1 level (11.77 +/− 1.32 ng/ml) and ET-1 | Hua Xi Yi Ke Da Xue |
| | level (78.37 +/− 17.54 pg/ml) were significantly | Xue Bao. 2001 |
| | higher in the patients than those in controls(P < | Jun; 32(2): 202-3, 212 |
| | 0.05) | |
| gastric cancer | It was found that TGF-beta 1 mRNA expression in | Korean J Intern Med. |
| | gastric cancer might concern the early stage of | 2002 Sep; 17(3): 160-6. |
| | gastric carcinogenesis and, it was higher in | |
| | patients with early gastric cancer, negative lymph- | |
| | nodes or negative perineural invasion. | |
| Liver dysfunction | chronic hepatitis B involving liver cell function | Zhonghua Gan Zang |
| | and liver fibrosis | Bing Za Zhi. 2002 |
| | | Jun; 10(3): 221-2. |
| human renal allograft | | Transplant Proc. 2001 |
| recipients | | Nov-Dec; 33(7-8): |
| | | 3148-50 |
| Kidney dysfunction | Diagnosis of chronic allograft nephropathy | Nephrol Dial |
| | | Transplant. 2001; 16 |
| | | Suppl 1: 114-6 |
| diabetic nephropathy | Forty-five days of diabetes resulted in increased | Horm Metab Res. |
| | albuminuria (p < 0.05), urinary TGF-beta 1 (p < | 2001 Nov; 33(11): 664-9 |
| | 0.05) and GLUT1 protein abundance (p < 0.05). | |
| | There was a positive correlation between urinary | |
| | TGF-beta 1 and plasma glucose levels (r = 0.65, p < | |
| | 0.05) and albuminuria (r = 0.72, p < 0.05). | |
| Systolic blood pressure | association between a transforming growth factor | J Appl Physiol. 2001 |
| at rest and in response | (TGF)-beta(1) gene polymorphism in codon 10 | Oct; 91(4): 1808-13. |
| to acute exercise | and blood pressure (BP) at rest, in acute response | |
| | to exercise | |
| prostate cancer- cancer | Plasma TGF-beta(1) levels are markedly elevated | J Clin Oncol. 2001 Jun |
| stage and prognosis in | in men with prostate cancer metastatic to regional | 1; 19(11): 2856-64 |
| patients who undergo | lymph nodes and bone. In men without clinical or | |
| radical prostatectomy | pathologic evidence of metastases, the | |
| | preoperative plasma TGF-beta(1) level is a strong | |
| | predictor of biochemical progression after surgery, | |
| | presumably because of an association with occult | |
| | metastatic disease present at the time of radical | |
| | prostatectomy. | |
| invasive prostate | Elevated plasma levels of TGF-beta 1 in patients | Nat Med. 1995 |
| cancer | with invasive prostate cancer | Apr; 1(4): 282-4. |
| | | J Cell Biochem Suppl. |
| | | 1992; 16H: 54-61 |
| ovarian carcinoma | expression of LTBP-1 and TGF-beta 1 mRNAs | Jpn J Cancer Res. |
| | was much higher in both serous and mucinous | 2001 May; 92(5): 506-15. |
| | adenocarcinomas than in their benign counterparts | Lab Invest. 1995 |
| | | Aug; 73(2): 213-20 |
| vulvar lichen sclerosus | Slight increase in TGF-beta 1 staining was seen in | J Reprod Med. 2001 |
| | the upper and mid-dermis in 6 of the 10 lichen | Feb; 46(2): 117-24. |
| | sclerosus specimens and in the morphea specimens | |
| | as compared to the control tissue, and this staining | |
| | was localized within cells | |
| gastric carcinoma | An elevated level of TGF-beta 1 was significantly | Anticancer Res. 2000 |
| | correlated with lymph node metastasis and poor | Nov-Dec; |
| | prognosis | 20(6B): 4489-93 |
| breast cancer | Of the 153 invasive breast cancer tissues, TGF | Anticancer Res. 2000 |
| | beta 1 was expressed strongly in 25 and | Nov-Dec; |
| | moderately in 98 cases. Immunostaining for | 20(6B): 4413-8 |
| | TGFb3 but not TGF beta 1 was inversely | |
| | correlated with overall survival (p = 0.0204). | |
| | When combined with lymph node involvement, | |
| | TGFb3 became an even more significant | |
| | prognostic factor for overall survival (p = 0.0003), | |
| breast cancer | TGF-beta 1 expression was found in 160/273 | Anticancer Res. 1995 |
| | (59%) and TGF-2 in 110/273 (40%) of tumour | Nov-Dec; |
| | specimens | 15(6B): 2627-31. |
| carcinoma of the cervix | In carcinoma of the cervix, pretreatment TGF beta | Int J Radiat Oncol Biol |
| | 1 levels reflect tumor burden and are a significant | Phys. 2000 Nov |
| | prognostic factor for survival | 1; 48(4): 991-5 |
| hypertension | A higher plasma TGF-beta(1) concentration is | Am J Hypertens. 2003 |
| | found in hypertensive patients with | Jul; 16(7): 604-11 |
| | microalbuminuria and left ventricle hypertrophy. | |
| Pseudoexfoliation | The immunolabeling of both intra- and extraocular | Klin Monatsbl |
| (PEX) syndrome | PEX deposits with antibodies against latent TGF- | Augenheilkd. 2000 |
| | beta 1 binding protein (LTBP-1) was particularly | Jun; 216(6): 412-9 |
| | prominent. In addition to the known intraocular | |
| | sites of PEX material accumulations, focal plaque- | |
| | like LTBP-1 positive deposits could be observed | |
| | in the conjunctival stroma, optic nerve meninges, | |
| | skin, heart muscle, lungs, kidney as well as in the | |
| | adventitia of the aorta and cerebral artery from | |
| | donors with PEX syndrome; such plaque-like | |
| | deposits positive for LTBP-1 were not present in | |
| | the control tissues | |
| pulmonary dysfunction | Patients with cystic fibrosis of a TGF-beta(1) high | Thorax. 2000 |
| in patients with cystic | producer genotype for codon 10 had more rapid | Jun; 55(6): 459-62 |
| fibrosis | deterioration in lung function than those with a | |
| | TGF-beta(1) low producer genotype | |
| multiple sclerosis | Biologically active TGF-beta 1 in serum was | Acta Neurol Scand. |
| | reduced in MS patients compared to controls, on | 1997 Aug; 96(2): 101-5 |
| | the other hand total TGF-beta 1 was elevated in | |
| | CSF compared to patients with OND. Biologically | |
| | active TGF-beta 1 in CSF correlated positively | |
| | with the duration of the acute relapse in patients | |
| | with primary-relapsing MS | |
| periodontal disease | In the patients, a significantly higher concentration | Eur J Oral Sci. 1997 |
| | of TGF-beta 1 was observed both in the gingival | Apr; 105(2): 136-42 |
| | tissues and fluid samples obtained from the sites | |
| | with deeper periodontal pockets than in the less | |
| | involved sites | |
| Multiple myeloma | | Acta Haematol. |
| | | 1997; 98(2): 116-8. |
| pulmonary | The survival rate was worse in patients in whom | Pathol Res Pract. 1996 |
| adenocarcinoma | each of the four growth factors was expressed than | Nov; 192(11): 1113-20 |
| | in those where growth factors were not expressed | |
| basal cell carcinoma | The stroma of most BCCs revealed enhanced | Br J Dermatol. 1996 |
| | TGF-beta 1 and T beta R II mRNA expression | Jun; 134(6): 1044-51 |
| | when compared with normal dermis | |
| progressive renal | Transgenic mice with increased plasma levels of | Lab Invest. 1996 |
| disease (progressive | TGF-beta 1 develop progressive renal disease | Jun; 74(6): 991-1003 |
| glomerulosclerosis) | | |
| progressive kidney | in rats given a second antibody injection, one week | Kidney Int. 1994 |
| fibrosis | later, the glomerular expression of TGF-beta 1 | Mar; 45(3): 916-27 |
| | mRNA and TGF-beta 1 protein remained elevated | |
| | through 18 weeks and was associated with a large | |
| | infiltration of mononuclear cells, with staining | |
| | features of fibroblastic/myofibroblastic cells, | |
| | strongly expressing TGF-beta 1 in the | |
| | tubulointerstitium of the kidney. | |
| renal allograft | Investigation demonstrated a significant | Kidney Int. 1996 |
| interstitial fibrosis and | correlation between intragraft TGF-beta 1 mRNA | May; 49(5): 1297-303 |
| chronic allograft | display and renal allograft interstitial fibrosis and | |
| nephropathy | chronic allograft nephropathy | |
| ischemic stroke/ | The ischemic penumbra contained the highest | Stroke. 1996 |
| ischemic brain tissue | levels of TGF-beta 1 mRNA, whereas the normal | May; 27(5): 852-7 |
| | contralateral hemispheres had the least (P < .001, | |
| | Mann-Whitney U test). | |
| diabetic nephropathy | Higher expression of TGF-beta 1 mRNA was | Kidney Int. 1996 |
| | observed in glomeruli of patients with diabetic | Apr; 49(4): 1120-6 |
| | nephropathy as compared with normal glomeruli. | |
| idiopathic pulmonary | TGF-beta 1 is expressed in epithelial cells of | Am J Respir Cell Mol |
| fibrosis | fibrotic lungs where the presence of TGF-beta 1 is | Biol. 1996 |
| | an indication of the chronicity of the injury. | Feb; 14(2): 131-8 |
| cardiac fibrosis | Very low expression of TGF-beta(1) and collagen | Mol Genet Metab. |
| | type I and III mRNA is seen in the normal rat | 2000 Sep-Oct; 71(1-2): |
| | heart. Both expressions are markedly increased in | 418-35 |
| | the infarcted heart and the levels of TGF-beta(1) | |
| | mRNA precedes increases in mRNA levels for | |
| | extracellular matrix (ECM) proteins, suggesting a | |
| | possible role of TGF-beta(1) in remodeling | |
| | processes in the myocardium. | |
| radiation-induced lung | TGF beta 1 has been implicated in the | Lung Cancer. 1998 |
| injury | development of normal tissue injury after | Feb; 19(2): 109-20. |
| | irradiation in several organs, including the lung. | |
| | Using the treatment of non-small cell lung cancer | |
| | as a model, we also discuss how it may be possible | |
| | to identify patients at risk for this complication | |
| | using measurements of plasma TGF beta | |
| progressive renal | For the reliable assessment of interstitial fibrosis it | Nephrol Dial |
| insufficiency | was found that the best correlating parameters of | Transplant. 2000; 15 |
| | interstitial fibrosis with renal function were: (i) the | Suppl 6: 72-3 |
| | ratio of protein accumulation of TGF-beta-1 and | |
| | its antagonist decorin; | |
| osteoporosis and | It was shown that a T869-->C polymorphism of | Pharmacogenetics. |
| vertebral fracture, and | the transforming growth factor-beta1 gene, which | 2001 Dec; 11(9): 765-71 |
| with the outcome of | results in a Leu-->Pro substitution at amino acid | |
| treatment for | 10, is associated with bone mineral density in | |
| osteoporosis with | Japanese adolescents and postmenopausal women, | |
| active vitamin D. | with genetic susceptibility to both osteoporosis and | |
| | vertebral fracture, and with the outcome of | |
| | treatment for osteoporosis with active vitamin D. | |
| obesity | Interestingly, the level of TGF-β mRNA was | Arterioscler Thromb |
| | significantly higher in the adipose tissue of both | Vasc Biol. 1998 |
| | ob/ob and db/db mice when compared with their | Jan; 18(1): 1-6. |
| | lean counterparts | |
| Crohn's disease (CD) | Both in CD and UC patients, increased expression | Ann Surg. 1999 |
| and ulcerative colitis | of all TGFbeta isoforms studied was detected in | Jan; 229(1): 67-75; |
| (UC) | the intestinal mucosa in the active phase of the | Gastroenterology. |
| | disease only. The correlation between TGFbeta | 1996 Apr; 110(4): 975-84 |
| | mRNA and severity of the inflammatory reaction | |
| | has also been demonstrated. | |
| Crohn's disease | There was a marked overexpression of TGF-beta | Langenbecks Arch |
| | 1, TGF-beta 3 and T beta R-II in 94% of the | Chir Suppl |
| | Crohn's disease tissue samples | Kongressbd. |
| | | 1998; 115: 994-7. |
| cholecystolithiasis | there was an enhanced TGF-beta1 mRNA | Langenbecks Arch |
| | expression (eightfold increase; P < 0.04) in the | Surg. 2005 |
| | cholecystolithiasis tissue samples in comparison | Feb; 390(1): 21-8. Epub |
| | with normal controls | 2004 Nov 20 |
| hepatic fibrosis | The extent of fibrosis correlated significantly with | Scand J Gastroenterol. |
| | the amount of collagen and TGFbeta1 mRNA | 2000 Sep; 35(9): 969-75 |
| | expression in liver tissues. The collagen content | |
| | and expression of TGFbeta1 mRNA were also | |
| | upregulated significantly in liver tissues with a | |
| | fibrosis score of 7 or more | |
| chronic hepatitis | In patient with chronic hepatitis TGF beta 1 | N Engl J Med. 1991 |
| | mRNA expression correlated closely with the | Apr 4; 324(14): 933-40 |
| | expression of procollagen Type I mRNA in the | |
| | liver (r = 0.94) and serum procollagen Type III | |
| | peptide (r = 0.89) | |
| Chronic hepatitis B and | In patients responding to therapy, TGF-beta1 | Scand J |
| C-response to | expression decreased in parallel with histological | Gastroenterol.2000 |
| INFalpha treatment | improvement, while no difference in TGF-beta1 | Dec; 35(12): 1294-300; |
| | expression was seen before and after treatment in | J. Hepatol, 1999; 30: |
| | non-responders | 1-7 |
| | | Cytokine, 1999: |
| | | 11: 1076-1080 |
| Liver function | Liver cirrhosis resulted in a significant increase of | Cytokine. 2000 |
| impairment in liver | plasma concentration of TGF-beta(1)(39.3 +/− 3.8 ng/ml), | Jun; 12(6): 677-81 |
| cirrhosis | which doubled normal values (18.3 +/− 1.6 ng/ml). | |
| | TGF-beta(1)level increased depending on | |
| | the degree of liver insufficiency. These findings | |
| | suggest possible use of plasma TGF- | |
| | beta(1)measurement as an indicator of liver | |
| | function impairment and possible marker of | |
| | hepatic fibrosis progression in cirrhotic patients | |
| food protein-induced | TGF-beta1 expression was generally depressed in | J Allergy Clin |
| enterocolitis syndrome | patients | Immunol. 2002 |
| (FPIES). | | Jan; 109(1): 150-4 |
| Atopic dermatitis (AD) | The odds ratio of children with AD having a low | J Allergy Clin |
| | TGFB1 producer genotype was 4.8 (95% CI, 2.4--9.7) | Immunol. 2001 |
| | compared with the control subjects (P < | Aug; 108(2): 281-4. |
| | .0001). TGFB1 genotype may partly explain the | |
| | strong genetic predisposition to AD. | |
| hemorrhagic colitis | Concentrations of TGF-beta1 were higher in | Am J Kidney Dis. |
| | children with uncomplicated O157: H7 HC than | 2000 Jan; 35(1): 29-34. |
| | among those who developed HUS (934 +/− 680 | |
| | versus 514 +/− 497 pg/mL, respectively; P < 0.04). | |
| renal parenchymal | There is an association between the TGF-beta1 −800 | Kidney Int. 2002 |
| scarring (RPS) | GA, −509 TT and Leu10-->Pro CT genotypes | Jan; 61(1): 61-7 |
| following childhood | and the presence or absence of RPS. The low | |
| urinary tract infection | TGF-beta1 producer status of the −800 GA | |
| (UTI) | genotype may protect against the development of a | |
| | pro-fibrotic pathology. | |
| celiac disease | The celiac mucosa showed weak aad patchy | J Pediatr Gastroenterol |
| | epithelial TGF-beta immunoreactivity. In contrast, | Nutr. 1999 |
| | an intense staining positivity was present in the | Sep; 29(3): 308-13 |
| | lamina propria localized mostly in the | |
| | subepithelial region where T cells, macrophages, | |
| | and CD25+ cells were detected by double staining. | |
| inflammatory bowel | the numbers of TGF-beta1-positive cells, including | Scand J Gastroenterol. |
| disease (IBD) in | T cells, neutrophils, and monocytes/macrophages, | 1999 Jun; 34(6): 591-600 |
| children | in the lamina propria increased during disease | |
| | activity. | |
| Henoch-Schonlein | TGF-beta-secreting T cells were significantly | Clin Exp Immunol. |
| purpura (HSP) | elevated during the acute stage, and decreased at | 2000 Nov; 122(2): 285-90 |
| | the convalescent stage | |
| salivary adenoid cystic | TGF beta 1 and TGF beta RI proteins were | Hunan Yi Ke Da Xue |
| carcinoma (ACC) | expressed in most ductal epithelial cells and myo- | Xue Bao. 2002 Jun |
| | epithelial cells, but they were not expressed in the | 28; 27(3): 229-32 |
| | acinar epithelial cells of any normal glands | |
| | examined. The contents of TGF beta 1 in ACC | |
| | were significantly higher than those in normal | |
| | salivary glands (P < 0.01), | |
| laryngeal squamous | It was found that invasive tumor growth of the | Anticancer Res. 1999 |
| cell carcinomas | larynx is associated with an increase of the TGF- | Sep-Oct; 19(5B): 4265-72 |
| | beta 1 protein and mRNA expression. The | |
| | synthesis of TGF-beta 1 is mainly performed by | |
| | the tumor cells, but also to a lesser extent by the | |
| | stroma cells. | |
| pediatric astrocytomas | Pilocytic, nonpilocytic, and anaplastic | Mol Chem |
| | astrocytomas have significant increased levels of | Neuropathol. 1998 |
| | IL-1 beta, IL-1RI, and TGF-beta 1 mRNAs, | Feb; 33(2): 125-37 |
| rheumatoid arthritis | TGF-beta 1 expression was significantly up- | Clin Immunol |
| | regulated on RA compared to OA and normal ST | Immunopathol. 1995 |
| | lining cells, interstitial macrophages, and | Aug; 76(2): 187-94 |
| | endothelial cells (P < 0.05). | |
| CHF/ | transition from LVH to CHF has been related to a | Rev Port Cardiol. 1999 |
| Transition from LVH | marked increase in microtubular intracytoplasmic | Jun; 18(6): 635-46. |
| to CHF | structure, the reduction of Ca++ ATPase | |
| | concentration of the sarcoplasmic reticulum, and | |
| | the increased myocardial expression of growth | |
| | factor TGF beta 1, which influences interstitial | |
| | fibrosis | |
| thoracic radiotherapy - | The TGF-beta(1) and IL-6 concentrations in the | Int J Radiat Oncol Biol |
| associated severe | BAL fluid recovered from the irradiated areas | Phys. 2004 Mar |
| pneumonitis | were significantly increased by thoracic RT. The | 1; 58(3): 758-67 |
| | increase in TGF-beta(1) levels tended to be greater | |
| | in the group of patients who developed severe | |
| | pneumonitis | |
| diffusely infiltrating | The expression of TGF beta 1 was markedly | Anticancer Res. 2002 |
| colorectal carcinoma | increased in DICC, especially of the schirrous | Nov-Dec; |
| (DICC) | type, compared to CCC | 22(6B): 3545-54 |
| Type 2 diabetes | In patients with type 2 diabetes mellitus both urine | Pol Arch Med Wewn. |
| mellitus AND diabetic | and serum TGF beta 1 concentration were | 2002 Aug; 108(2): 745-52 |
| nephropathy in the | elevated. A positive correlation between urine | |
| course of diabetes | TGF beta 1 level and the progression of renal | |
| mellitus | failure was observed. In conclusion, urine TGF | |
| | beta 1 level may be a good prognostic factor of the | |
| | development of diabetic nephropathy in the course | |
| | of diabetes mellitus | |
| disturbed placentation | Leptin and TGF-beta(1) concentrations were lower | Placenta. 2002 |
| in women destined to | and PAI-2 concentration higher in women destined | May; 23(5): 380-5 |
| develop pre-eclampsia | to develop pre-eclampsia relative to controls | |
| transitional cell | plasma TGF-beta(1) levels are elevated in patients | Cancer. 2001 Dec |
| carcinoma (TCC) of | with muscle-invasive TCC before cystectomy. | 15; 92(12): 2985-92 |
| the urinary bladder | Transforming growth factor-beta(1) levels are | |
| | highest in patients with bladder carcinoma | |
| | metastatic to lymph nodes and are a strong | |
| | independent predictor of disease recurrence and | |
| | disease specific mortality | |
| Sepsis following | Thermal injury induces a significant increase in | Shock. 2001 |
| thermal injury | serum TGF-beta, which may contribute to post- | Nov; 16(5): 380-2. |
| | burn immunosuppression with an increased | |
| | susceptibility to sepsis | |
| liver fibrosis of chronic | The damage of DNA in hepatocytes of CH | Zhonghua Shi Yan He |
| viral hepatitis (CH). | correlated closely with the expression of Fas and | Lin Chuang Bing Du |
| | TGF-beta 1 in liver tissue and serum. In severe | Xue Za Zhi. 2000 |
| | type CH and liver cirrhosis, the degree of liver | Mar; 14(1): 31-3. |
| | fibrosis and the expression of P III P and TGF-beta | |
| | 1 were higher than that in mild type and moderate | |
| | type CH | |
| discriminating patients | The ability of serum HA, LN and TGF-beta(1) to | Zhonghua Gan Zang |
| of chronic hepatitis | differentiate patients with extensive liver fibrosis | Bing Za Zhi. 2001 |
| with “no liver fibrosis”, | from those with no or mild liver fibrosis exceeded | Jun; 9(3): 148-50 |
| “liver fibrosis but no | that of serum PCIII and CIV (the areas under the | |
| cirrhosis” and “liver | curves = 0.849, 0.819, 0.836 vs 0.702, 0.721, | |
| cirrhosis” | P < 0.05. | |
| chronic idiopathic | serum interleukin-1 beta (IL-1 beta), tumor | Am J Hematol. 2000 |
| neutropenia of adults | necrosis factor-alpha (TNF-alpha), interleukin-6 | Dec; 65(4): 271-7 |
| (CINA) | (IL-6), transforming growth factor-beta(1) (TGF- | |
| | beta(1)), and soluble tumor necrosis factor | |
| | receptor p55 (sTNF-RI) were all significantly | |
| | increased in CINA patients compared to controls | |
| primary hepatic | The result revealed that serum TGF beta 1 level of | Zhonghua Nei Ke Za |
| carcinoma (PHC) | PHC patients were significantly higher (257.6 +/− | Zhi. 1997 |
| | 126.0 micrograms/L) than those in normal subjects | Oct; 36(10): 669-72 |
| | (81.5 +/− 43.5 micrograms/L). Above results | |
| | suggest that serum TGF beta 1 might be a | |
| | candidate for a novel tumor marker for diagnosis | |
| | and monitoring of PHC | |
| dengue haemorrhagic | plasma obtained from children with DHF had | Trans R Soc Trop Med |
| fever (DHF) in | significantly higher levels of TGF beta-1 than | Hyg. 1998 Nov-Dec; |
| children with dengue | plasma from children with DF, especially from | 92(6): 654-6 |
| virus infection | days 1 to 3 after the onset of fever | |
| Preterm neonates with | TGF beta 1 levels were lower in patients with | Allergol |
| respiratory distress | CLD and they showed undetectable values in 8 | Immunopathol (Madr). |
| syndrome (RDS) with | samples. | 1999 Jan-Feb; |
| chronic lung disease | | 27(1): 11-7 |
| (CLD). | | |
| tarsal and limbal forms | Increased levels of TGF-beta 1, IL-1 and IL-6 in | Exp Eye Res. 1998 |
| of vernal | VKC tissues and tears indicate a local production | Jul; 67(1): 105-12. |
| keratoconjunctivitis | of these cytokines in active VKC | |
| (VKC) | | |
| bullous pemphigoid | an opposite behavior was observed for | Br J Dermatol. 1998 |
| (BP) | transforming growth factor beta 1 (TGF-beta 1), | Apr; 138(4): 611-4 |
| | whose serum levels were higher in BP than the | |
| | concentrations in BF | |
| ureteropelvic junction | The level of TGFbeta1 is elevated in the renal | Urology. 1997 |
| obstruction (UPJO). | pelvis of children with UPJO compared to the | Nov; 50(5): 769-73. |
| | level in the bladder of either obstructed or | |
| | nonobstructed upper urinary tracts. | |
| hepatocellular | The results indicate that there were significantly | Medicine (Baltimore). |
| carcinoma (HCC) | increased urinary TGF-beta 1 levels in patients | 1997 May; 76(3): 213-26 |
| | with HCC. Raised TGF-beta 1 levels were | |
| | associated, in a dose-related fashion, with | |
| | increased risk for development of HCC (odds | |
| | ratio, 1.05, 95% confidence interval, 1.03-1.07). | |
| | HCC patients with raised TGF-beta 1 levels had | |
| | shorter survival | |
| coronary artery disease | naturally active TGF-beta 1 was significantly | Cardiovasc Res. 1997 |
| (CAD) | higher in CAD patients (1.74 +/− 0.18 vs 0.96 +/− | May; 34(2): 404-10. |
| | 0.17 ng/ml, P < 0.01). An increase in active TGF- | |
| | beta 1 levels was identified with both the | |
| | occurrence and severity of CAD which is | |
| | independent of standard CAD risk factors | |
| Lung cancer | Elevated TGF beta 1 were found in 50% (27/54) of | Lung Cancer. 1996 |
| | lung cancer patients. During radiation therapy | Dec; 16(1): 47-59 |
| | plasma TGF beta 1 levels declined, however, by | |
| | the completion of treatment the mean TGF beta 1 | |
| | level had not normalized in patients with lung | |
| | cancer. The TGF beta 1 level at last follow-up | |
| | correlated with disease status in those patients with | |
| | an increased pretreatment plasma level. | |
| Non-Insulin Dependent | TGF-beta 1 was significantly elevated in NIDDM | Diabetes Care. 1996 |
| Diabetes Mellitus | (7.9 +/− 1.0 ng/ml), as compared with control | Oct; 19(10): 1113-7 |
| | subjects (3.1 +/− 0.4 ng/ml, P < 0.001) | |
| colorectal tumours | In 86% of the tumors the LAP-TGF-beta complex | Br J Cancer. 1996 |
| | was present in both the stromal and epithelial cells, | Sep; 74(5): 753-8 |
| | whereas the mature TGF-beta 1 peptide was | |
| | expressed in the glandular epithelium of 58.3% of | |
| | these tumours. Intense staining for TGF-beta 1 was | |
| | positively associated with advanced Dukes' stage. | |
| | Furthermore, there was a significant correlation | |
| | between the presence of TGF-beta 1 in the | |
| | tumours and a shorter post-operative survival. | |
| anti-neutrophil | the TGF-beta 1 isoform was found to be over- | Clin Exp Immunol. |
| cytoplasmic antibody | expressed in SV (systemic vasculitides), including | 1996 Jul; 105(1): 104-11 |
| (ANCA)-associated | AAV, and to correlate with disease activity as | |
| vasculitis (AAV) | shown for WG (Wegener granulomatosis). Mean | |
| | TGF-beta 1 plasma levels in AAV patients ranged | |
| | from 8.9 ng/ml (WG) to 13.3 ng/ml (CSS) (control | |
| | 4.2 ng/ml; P < 0.01) | |
| cervical carcinoma of | Percent positive staining for the intracellular form | Cancer. 1996 Mar |
| the uterine | of TGF-beta 1 was 100% for normal epithelium, | 15; 77(6): 1107-14 |
| | 73.3% for CIN, and 44.1% for invasive | |
| | carcinomas, (P = 0.002). Percent positive staining | |
| | for the extracellular form of TGF-beta 1 was | |
| | 63.6% for stroma underlying normal epithelium, | |
| | 60% for stroma associated with CIN, and 94.1% | |
| | for stroma surrounding invasive cancer (P = 0.007) | |
| Pancreatic cancer | TGF-beta 1 inhibits the generation of the | Surgery. 1998 |
| | antiangiogenic molecule angiostatin by human | Aug; 124(2): 388-93 |
| | pancreatic cancer cells in a time- and dose- | |
| | dependent manner | |
| Autoimmune diseases | Increased TGF-betal production correlates with | J Autoimmun. 2000 |
| | protection and/or recovery from autoimmune | Feb; 14(1): 23-42 |
| | diseases. TGF-betal levels are expected to be | |
| | related to the extent of autoimmune process | |

According to other optional embodiments of the present invention, variants of this cluster according to the present invention (amino acid and/or nucleic acid sequences of HSTGFB1) may optionally have one or more of the following utilities, some of which are related to utilities described above. It should be noted that these utilities are optionally and preferably suitable for human and nonhuman animals as subjects, except where otherwise noted.

A non-limiting example of such a utility is monitoring effectiveness of topically applied androgens in Sjogren's syndrome by detecting a HSTGFB1 variant according to the present invention, optionally and preferably in tears when such androgens are topically applied to the eye. The level of such a variant according to the present invention may optionally be correlated with the effectiveness of such treatment for symptomatic relief of ocular dryness (keratoconjunctivitis sicca (KCS)), a typical complication of Sjogren's syndrome.

Use of the known protein for such a diagnostic utility is described with regard to EP0831868, hereby incorporated by reference as if fully set forth herein.

Another non-limiting example of such a utility is the detection of an increased susceptibility to osteoporosis by detecting a HSTGFB1 variant according to the present invention. The known protein, TGF-β1, is released from bone matrix during bone resorption and subsequently activated by the low pH below the ruffled border of the resorbing osteoclasts. TGF-β1 has been implicated as a possible mediator of coupling between bone resorption and formation because: it inhibits mature osteoclasts and proliferation of mononuclear osteoclast precursors in vitro; the peptide inhibits fusion of mononuclear precursors into osteoclasts; TGF-β1 has been found to stimulate proliferation or differentiation of preosteoblasts in vitro; and bone matrix has the highest concentrations of TGF-β1 of all tissues. A particular variation in the human TGF-β1 gene, the 713-8delC sequence variation, has been shown to be correlated with an altered osteoporosis risk, as described with regard to EP0955378, hereby incorporated by reference as if fully set forth herein.

Another non-limiting example of such a utility is the diagnosis of diseases including, but not limited to, a variety of cancers, including but not limited to breast cancer, colorectal cancer and classical Hodgkin's Lymphoma (cHL), fibrosis, suppression of cell-mediated immunity, glaucoma and diffuse systemic sclerosis.

Use of the known protein for such a diagnostic utility is described with regard to WO 04/113522, hereby incorporated by reference as if fully set forth herein.

Another non-limiting example of such a utility is the diagnosis of susceptibility to renal disease, optionally and preferably including prognosis of progression of renal disease and/or progression to renal failure, optionally including chronic renal failure (CRF) and/or ESRF. Optionally, the diagnosis may include determination of the prognosis of renal disease and/or renal failure from a variety of diseases and/or conditions, including but not limited to, hypertension (optionally including but not limited to systemic hypertension and/or essential hypertension), diabetes mellitus, vascular diseases, systemic lupus erythematosus, obstruction to the urinary tract or inflammation of the tubular system (pyelonephritis) and/or increased age. CRF is defined as an irreversible, long-standing loss of renal function. End stage renal failure (ESRF) is an advanced form of CRF and refers to advanced renal insufficiency when renal function is approximately 10% of normal prior to the initiation of either dialysis or renal transplantation. The incidence of renal failure from various renal diseases increases with age; vascular diseases and diabetes mellitus are the most common causes of ESRD in the elderly. CRF can also arise as a complication of systemic lupus erythematosus, obstruction to the urinary tract or inflammation of the tubular system (pyelonephritis). Systemic hypertension has been shown to be one of the major factors contributing to the deterioration of renal function and elevated blood pressure can be a cause or a consequence of renal injury. The incidence of systemic hypertension in patients with serious renal disease is 90%. Furthermore the incidence of renal failure in patients with non-accelerated essential (no apparent cause) hypertension is about 10%. Polymorphisms in the human TGF-β1 gene have been shown to correlate with increased risk of renal failure, as described in WO 02/090585, hereby incorporated by reference as if fully set forth herein.

HNRPUL1 (NM_{—}007040; NP_{—}008971) is antisense, tail to tail, to TGF-β1 and may therefore be co-regulated with one or more HSTGFB1 variants according to the present invention, and hence may have one or more utilities of HSTGFB1 variants according to the present invention as described herein. HNRPUL1 is E1B-55 kDa-associated protein (E1B-AP5). The adenovirus type 5 (Ad5) early 1B 55-kDa protein (E1B-55 kDa) is a multifunctional phosphoprotein that regulates viral DNA replication and nucleocytoplasmic RNA transport in lytically infected cells. In addition, E1B-55 kDa provides functions required for complete oncogenic transformation of rodent cells in cooperation with the E1A proteins. HNRPUL1 is a nuclear RNA-binding protein of the heterogeneous nuclear ribonucleoprotein (hnRNP) family. Two distinct segments in the 55-kDa polypeptide which partly overlap regions responsible for p53 binding are required for complex formation with E1B-AP5 in Ad-infected cells. Stable expression of E1B-AP5 in Ad-infected cells overcomes the E1B-dependent inhibition of cytoplasmic host mRNA accumulation. The data suggest that E1B-AP5 might play a role in RNA transport and that this function is modulated by E1B-55 kDa in Ad-infected cells. (Gabler S et al. J Virol. 1998 October; 72(10):7960-71). Thus, HSTGFB1 expression might serve as a marker for oncogenic transformation and adenovirus infection.

Table 179 below describes diagnostic utilities for the cluster HSTGFB1 that were found through microarrays, including the statistical significance thereof and a reference. One or more HSTGFB1 variants according to the present invention may optionally have one or more of these utilities.

TABLE 179

| | Statistical | |
|---|---|---|
| Diagnostic utility | significance | reference |
| Gene over expressed in | 6.2E−8 to | Su AI, Hampton GM Cancer Res (2001) Molecular |
| Clear Cell Renal Cell | 8.8E−4 | Classification of Human Carcinomas by Use of Gene |
| Carcinoma (vs. normal | | Expression Signatures. |
| kidney and other cancer | | Higgins JP, Brooks JD. Am J Pathol (2003) Gene |
| types). | | expression patterns in renal cell carcinoma assessed |
| | | by complementary DNA microarray. |
| Gene over expressed in | 8.4E−10 | Armstrong SA, Korsmeyer SJ Nat Genet (2002) |
| Mixed Lineage Leukemia | | MLL translocations specify a distinct gene |
| (vs. Acute Lymphocyctic | | expression profile that distinguishes a unique |
| Leukemia, Acute | | leukemia. |
| Myelogenous Leukemia) | | |
| Gene over expressed in lung | 3E−4 to 0.04, | Beer DJ, Hanash SM Nature Medicine (2002) Gene- |
| Adenocarcinoma and | 1.1E−4, 3.5E−5 | expression profiles predict survival of patients with |
| carcinoid and under | to 0.026 | lung adenocarcinoma. |
| expressed in Squamous Cell | respectively. | Garber ME, Petersen I PNAS (2001) Diversity of |
| Lung Carcinoma. | | gene expression in adenocarcinoma of the lung. |
| Differential diagnosis of lung | | |
| cancer subtypes. | | |
| Gene over expressed in | 5.6E−4, 4.8E−9 | Dhanasekaran SM, Chinnaiyan AM Nature (2001) |
| Benign Prostatic Hyperplasia | | Delineation of prognostic biomarkers in prostate |
| (vs. normal prostate) and | | cancer. |
| under expressed in prostate | | |
| cancer (vs. BPH and normal | | |
| cancer). Differential | | |
| diagnosis of prostate BPH vs. | | |
| prostate cancer. | | |
| Gene under expressed in | | GNF database |
| CD3+ cells of Idiopathic | | (http://www.ncbi.nlm.nih.gov/projects/geo/): |
| thrombocytopenic purpura | | GDS390, probe ID: 41445_at |
| patients, normal expression | | |
| in relapse. Differential | | |
| diagnosis of primary ITP vs. | | |
| relapse. | | |

Also, microarrays have shown that one or more HSTGFB1 variants according to the present invention may be overexpressed in lung cancer, preferably non small cell lung cancer, optionally adenocarcinoma or squamous cell cancer, preferably carcinoid.

A-to-I RNA editing is the site-specific modification of adenosine to inosine in stem-loop structures within precursor messenger RNAs. It is essential for normal development, and associated with a number of human diseases. Editing was considered a rare phenomenon, affecting only a handful of targets. However, it has recently been reported that editing of human transcripts is abundant, affecting thousands of genes, and up to 1 in every 2000 nt. The complete functional implications of this phenomenon are still being clarified, but the editing level can serve as a diagnostic marker for neurological diseases and cancers.

Most human editing sites reside within the primate-specific Alu repeats, and therefore all the exon with Alu repeats have the potential to be edited. The present inventors found such repeats within the exons of TGFB1. Therefore, this sequence should be edited.

Since altered editing patterns have been found to be associated with inflammation

Patterson, J. B. et al., 1995 Mol. Cell. Biol., 15, 5376-5388), epilepsy (Brusa, R., et al., 1995 Science, 270, 1677-1680), depression (Gurevich, I., et al., 2002 Neuron, 34, 349-356), ALS, (Kawahara, Y., et al., 2004 Nature, 427, 801) and malignant gliomas (Maas, S., et al., Proc. Natl. Acad. Sci. USA, 98, 14687-14692), the levels of the editing in TGFB1 mRNA may serve as potential diagnostic marker.

Other non-limiting exemplary utilities for HSTGFB1 variants according to the present invention are described in greater detail below and also with regard to the previous section on clinical utility.

Cluster HSTGFB1 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

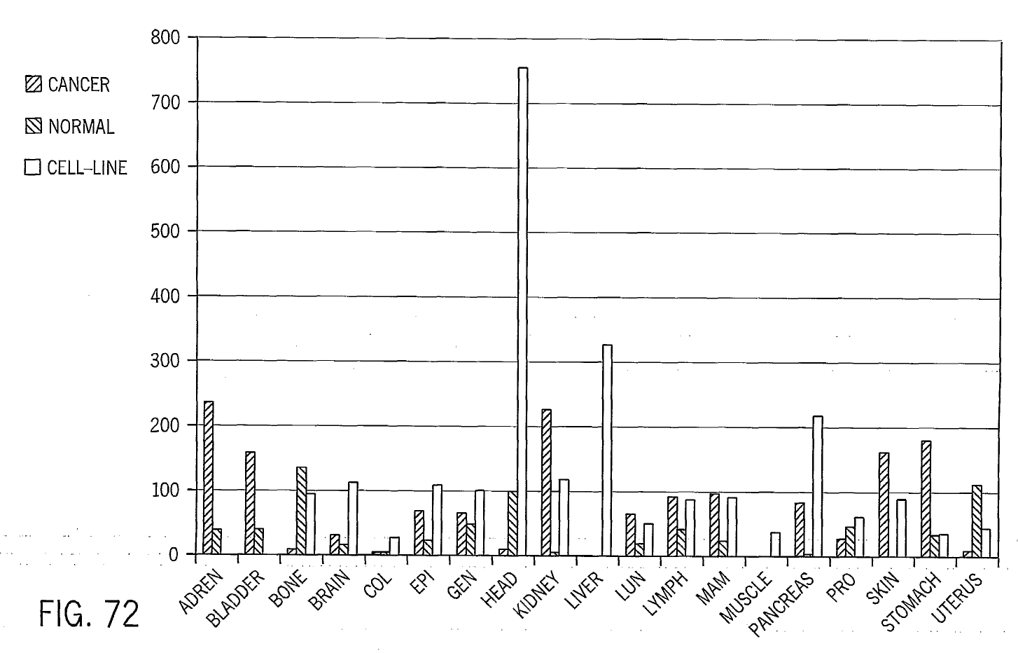

Overall, the following results were obtained as shown with regard to the histograms in FIG. 72 and Table 180. This cluster is overexpressed (at least at a minimum level) in the following pathological conditions: epithelial malignant tumors, kidney malignant tumors, pancreas carcinoma and skin malignancies. P values and ratios for expression in cancerous tissue are described in table 181.

TABLE 180
Normal tissue distribution

| Name of Tissue | Number |
|---|---|
| adrenal | 40 |
| bladder | 41 |
| bone | 136 |
| brain | 18 |
| colon | 6 |
| epithelial | 25 |
| general | 51 |
| head and neck | 101 |
| kidney | 6 |
| liver | 0 |
| lung | 21 |
| lymph nodes | 43 |
| breast | 26 |
| muscle | 0 |
| pancreas | 4 |
| prostate | 48 |
| skin | 0 |
| stomach | 36 |
| uterus | 113 |

TABLE 181
P values and ratios for expression in cancerous tissue

| Name of Tissue | P1 | P2 | SP1 | R3 | SP2 | R4 |
|---|---|---|---|---|---|---|
| adrenal | 1.5e−01 | 2.1e−01 | 7.5e−02 | 2.9 | 1.5e−01 | 2.2 |
| bladder | 3.3e−01 | 4.5e−01 | 1.8e−01 | 2.4 | 3.8e−01 | 1.7 |
| bone | 6.8e−01 | 7.3e−01 | 1 | 0.2 | 9.0e−01 | 0.6 |
| brain | 1.8e−01 | 1.1e−01 | 3.1e−01 | 1.6 | 7.1e−03 | 2.1 |
| colon | 7.1e−01 | 5.3e−01 | 1 | 1.0 | 7.7e−01 | 1.2 |
| epithelial | 5.1e−02 | 5.1e−03 | 5.6e−05 | 2.2 | 5.8e−09 | 3.1 |
| general | 2.3e−01 | 3.6e−02 | 5.4e−02 | 1.1 | 3.1e−05 | 1.5 |
| head and neck | 4.6e−01 | 4.3e−01 | 1 | 0.6 | 5.0e−02 | 0.8 |
| kidney | 4.3e−01 | 4.6e−01 | 5.1e−04 | 3.0 | 1.1e−03 | 3.8 |
| liver | 1 | 1.9e−01 | 1 | 1.0 | 8.5e−03 | 2.8 |
| lung | 4.5e−01 | 3.0e−01 | 1.9e−01 | 2.5 | 1.4e−01 | 2.2 |
| lymph nodes | 6.9e−01 | 7.5e−01 | 3.2e−01 | 1.8 | 2.7e−01 | 1.3 |
| breast | 6.5e−01 | 4.0e−01 | 1.5e−01 | 1.8 | 1.4e−01 | 1.7 |
| muscle | 2.9e−01 | 9.5e−02 | 1 | 1.0 | 3.9e−01 | 2.6 |
| pancreas | 3.5e−02 | 4.8e−03 | 5.7e−03 | 6.6 | 1.3e−04 | 7.8 |
| prostate | 8.6e−01 | 8.6e−01 | 8.0e−01 | 0.7 | 6.8e−01 | 0.8 |
| skin | 2.3e−01 | 2.3e−02 | 2.0e−02 | 13.1 | 3.2e−03 | 6.0 |
| stomach | 5.0e−01 | 7.5e−01 | 1.1e−01 | 1.5 | 4.3e−01 | 1.1 |
| uterus | 7.9e−01 | 8.0e−01 | 1 | 0.3 | 9.9e−01 | 0.3 |

As noted above, cluster HSTGFB1 features 6 transcript(s), which were listed in Table 174 above. These transcript(s) encode for protein(s) which are variant(s) of protein Transforming growth factor beta 1 precursor (SEQ ID NO:463). A description of each variant protein according to the present invention is now provided.

Variant protein HSTGFB1_P2 (SEQ ID NO:464) according to the present invention has an amino acid sequence; it is encoded by transcript(s) HSTGFB1_T5 (SEQ ID NO:433). An alignment is given to the known protein (Transforming growth factor beta 1 precursor (SEQ ID NO:463)) at. One or more alignments to one or more previously published protein sequences are given in the alignment table located on the attached CDROM. A brief description of the relationship of the variant protein according to the present invention to each such aligned protein is as follows:

Comparison report between HSTGFB1_P2 (SEQ ID NO:464) and TGFB1_HUMAN (SEQ ID NO:463):

1. An isolated chimeric polypeptide encoding for HSTGFB1_P2 (SEQ ID NO:464), comprising a first amino acid sequence being at least 90% homologous to MPPSGLRLLLLLLPLLWLLVLTPGRPAAGLSTCKTIDMELVKRKRIEAIRGQILSKLRLASPPSQGEV PPGPLPEAVLALYNSTRDRVAGESAEPEPEPEADYYAKEVTRVLMVETHNEIYDKFKQSTHSIYMF FNTSELREAVPEPVLLSRAELRLLRLKLKVEQHVELYQKYSNNSWRYLSNRLLAPSDSPEWLSFDV TGVVRQWLSRGGEIEGFRLSAHCSCDSRDNTLQVDING corresponding to amino acids 1-238 of TGFB1_HUMAN (SEQ ID NO:463), which also corresponds to amino acids 1-238 of HSTGFB1_P2 (SEQ ID NO:464), and a second amino acid sequence being at least 70%, optionally at least 80%, preferably at least 85%, more preferably at least 90% and most preferably at least 95% homologous to a polypeptide having the sequence EACFPGHAQL (SEQ ID NO: 661) corresponding to amino acids 239-248 of HSTGFB1_P2 (SEQ ID NO:464), wherein said first amino acid sequence and second amino acid sequence are contiguous and in a sequential order.

2. An isolated polypeptide encoding for a tail of HSTGFB1_P2 (SEQ ID NO:464), comprising a polypeptide being at least 70%, optionally at least about 80%, preferably at least about 85%, more preferably at least about 90% and most preferably at least about 95% homologous to the sequence EACFPGHAQL (SEQ ID NO: 661) in HSTGFB1_P2 (SEQ ID NO:464).

The location of the variant protein was determined according to results from a number of different software programs and analyses, including analyses from SignalP and other specialized programs. The variant protein is believed to be located as follows with regard to the cell: secreted.

The glycosylation sites of variant protein HSTGFB1_P2 (SEQ ID NO:464), as compared to the known protein Transforming growth factor beta 1 precursor (SEQ ID NO:463), are described in Table 182 (given according to their position(s) on the amino acid sequence in the first column; the second column indicates whether the glycosylation site is present in the variant protein; and the last column indicates whether the position is different on the variant protein).

TABLE 182
Glycosylation site(s)

| Position(s) on known | Present in | Position in |
|---|---|---|
| amino acid sequence | variant protein? | variant protein? |
| 136 | yes | 136 |
| 82 | yes | 82 |
| 176 | yes | 176 |

The variant protein has the following domains, as determined by using InterPro. The domains are described in Table 183:

TABLE183
InterPro domain(s)

| InterPro ID | Domain description | Analysis type | Position(s) on protein |
|---|---|---|---|
| IPR003911 | Transforming growth factor | FPrintScan | 163-177, 17-36, 178-193, |
| | beta TGFb | | 195-209, 72-84 |
| IPR003939 | Transforming growth factor, | FPrintScan | 12-31, 135-154, 166-177, |
| | beta 1 | | 207-219, 34-43 |
| IPR001111 | Transforming growth factor | HMMPfam | 33-238 |
| | beta (TGFb), N-terminal | | |

Variant protein HSTGFB1_P2 (SEQ ID NO:464) is encoded by the following transcript(s): HSTGFB1_T5 (SEQ ID NO:433). The coding portion of transcript HSTGFB1_T5 (SEQ ID NO:433) starts at position 1038 and ends at position 1781. The transcript also has the following SNPs as listed in Table 184 (given according to their position on the nucleotide sequence, with the alternative nucleic acid listed; the last column indicates whether the SNP is known or not; the presence of known SNPs in variant protein HSTGFB1_P2 (SEQ ID NO:464) sequence provides support for the deduced sequence of this variant protein according to the present invention).

TABLE 184
Nucleic acid SNPs

| SNP position on nucleotide | Alternative | Previously |
|---|---|---|
| sequence | nucleic acid | known SNP? |
| 181 | C -> A | Yes |
| 205 | C -> T | Yes |
| 206 | T -> A | Yes |
| 211 | G -> C | Yes |
| 214 | G -> | No |
| 289 | C -> | No |
| 390 | G -> C | Yes |
| 585 | G -> A | Yes |
| 651 | C -> T | Yes |
| 762 | C -> A | Yes |
| 789 | C -> A | Yes |
| 896 | C -> T | Yes |
| 1024 | G -> A | Yes |
| 1030 | C -> | No |
| 1030 | C -> T | No |
| 1066 | T -> C | Yes |
| 1111 | G -> C | Yes |
| 1177 | A -> G | Yes |
| 1232 | G -> | No |
| 1443 | A -> C | No |
| 1725 | A -> | No |
| 1725 | A -> T | No |
| 1964 | C -> T | Yes |
| 2139 | C -> T | Yes |
| 2148 | G -> A | Yes |
| 2149 | C -> | No |
| 2254 | G -> | No |
| 2254 | G -> C | No |
| 2255 | C -> | No |
| 2255 | C -> G | No |
| 2286 | G -> | No |
| 2317 | A -> | No |
| 2317 | A -> C | No |
| 2353 | C -> T | No |
| 2408 | C -> T | No |

Variant protein HSTGFB1_P3 (SEQ ID NO:465) according to the present invention has an amino acid sequence; it is encoded by transcript(s) HSTGFB1_T6 (SEQ ID NO:434). An alignment is given to the known protein (Transforming growth factor beta 1 precursor (SEQ ID NO:463)). One or more alignments to one or more previously published protein sequences are given in the alignment table located on the attached CDROM. A brief description of the relationship of the variant protein according to the present invention to each such aligned protein is as follows:

Comparison report between HSTGFB1_P3 (SEQ ID NO:465) and TGFB1_HUMAN (SEQ ID NO:463):

1. An isolated chimeric polypeptide encoding for HSTGFB1_P3 (SEQ ID NO:465), comprising a first amino acid sequence being at least 90% homologous to MPPSGLRLLLLLLPLLWLLVLTPGRPAAGLSTCKTIDMELVKRKRIEAIRGQILSKLRLASPPSQGEV PPGPLPEAVLALYNSTRDRVAGESAEPEPEPEADYYAKEVTRVLMVETHNEIYDKFKQSTHSIYMF FNTSELREAVPEPVLLSRAELRLLRLKLKVEQHVELYQKYSNNSWRYLSNRLLAPSDSPEWLSFDV TGVVRQWLSRGGEIEGFRLSAHCSCDSRDNTLQVDINGFTTGRRGDLATIHGMNRPFLLLMATPLE RAQHLQSSRHRRALDTNYCFSSTEKNCCVRQLYIDFRKDLGWKWIHEPKGYHANFCLGPCPYIWS LDTQYSKV corresponding to amino acids 1-339 of TGFB1_HUMAN (SEQ ID NO:463), which also corresponds to amino acids 1-339 of HSTGFB1_P3 (SEQ ID NO:465), and a second amino acid sequence being at least 70%, optionally at least 80%, preferably at least 85%, more preferably at least 90% and most preferably at least 95% homologous to a polypeptide having the sequence RLAHRATRCAWGEPGRRKRREKEK (SEQ ID NO: 662) corresponding to amino acids 340-363 of HSTGFB1_P3 (SEQ ID NO:465), wherein said first amino acid sequence and second amino acid sequence are contiguous and in a sequential order.

2. An isolated polypeptide encoding for a tail of HSTGFB1_P3 (SEQ ID NO:465), comprising a polypeptide being at least 70%, optionally at least about 80%, preferably at least about 85%, more preferably at least about 90% and most preferably at least about 95% homologous to the sequence RLAHRATRCAWGEPGRRKRREKEK (SEQ ID NO: 662) in HSTGFB1_P3 (SEQ ID NO:465).

The location of the variant protein was determined according to results from a number of different software programs and analyses, including analyses from SignalP and other specialized programs. The variant protein is believed to be located as follows with regard to the cell: secreted.

The glycosylation sites of variant protein HSTGFB1_P3 (SEQ ID NO:465), as compared to the known protein Transforming growth factor beta 1 precursor (SEQ ID NO:463), are described in Table 185 (given according to their position(s) on the amino acid sequence in the first column; the second column indicates whether the glycosylation site is present in the variant protein; and the last column indicates whether the position is different on the variant protein).

TABLE 185
Glycosylation site(s)

| Position(s) on known | Present in | |
|---|---|---|
| amino acid sequence | variant protein? | Position in variant protein? |
| 136 | yes | 136 |
| 82 | yes | 82 |
| 176 | yes | 176 |

The variant protein has the following domains, as determined by using InterPro. The domains are described in Table 186:

TABLE 186
InterPro domain(s)

| InterPro ID | Domain description | Analysis type | Position(s) on protein |
|---|---|---|---|
| IPR003911 | Transforming growth factor | FPrintScan | 163-177, 17-36, 178-193, |
| | beta TGFb | | 195-209, 72-84 |
| IPR003939 | Transforming growth factor, | FPrintScan | 12-31, 135-154, 166-177, |
| | beta 1 | | 207-219, 264-275, 34-43 |
| IPR001839 | Transforming growth factor | HMMPfam | 290-341 |
| | beta | | |
| IPR001111 | Transforming growth factor | HMMPfam | 33-252 |
| | beta (TGFb), N-terminal | | |
| IPR001839 | Transforming growth factor | HMMSmart | 293-351 |
| | beta | | |
| IPR001839 | Transforming growth factor | ScanRegExp | 311-326 |
| | beta | | |
| IPR001839 | Transforming growth factor | BlastProDom | 279-339 |
| | beta | | |
| IPR001839 | Transforming growth factor | ProfileScan | 289-328 |
| | beta | | |

Variant protein HSTGFB1_P3 (SEQ ID NO:465) is encoded by the following transcript(s): HSTGFB1_T6 (SEQ ID NO:434). The coding portion of transcript HSTGFB1_T6 (SEQ ID NO:434) starts at position 1038 and ends at position 2126. The transcript also has the following SNPs as listed in Table 187 (given according to their position on the nucleotide sequence, with the alternative nucleic acid listed; the last column indicates whether the SNP is known or not; the presence of known SNPs in variant protein HSTGFB1_P3 (SEQ ID NO:465) sequence provides support for the deduced sequence of this variant protein according to the present invention).

TABLE 187
Nucleic acid SNPs

| SNP position on | Alternative | Previously |
|---|---|---|
| nucleotide sequence | nucleic acid | known SNP? |
| 181 | C -> A | Yes |
| 205 | C -> T | Yes |
| 206 | T -> A | Yes |
| 211 | G -> C | Yes |
| 214 | G -> | No |
| 289 | C -> | No |
| 390 | G -> C | Yes |
| 585 | G -> A | Yes |
| 651 | C -> T | Yes |
| 762 | C -> A | Yes |
| 789 | C -> A | Yes |
| 896 | C -> T | Yes |
| 1024 | G -> A | Yes |
| 1030 | C -> | No |
| 1030 | C -> T | No |
| 1066 | T -> C | Yes |
| 1111 | G -> C | Yes |
| 1177 | A -> G | Yes |
| 1232 | G -> | No |
| 1443 | A -> C | No |
| 1725 | A -> | No |
| 1725 | A -> T | No |
| 1825 | C -> T | Yes |
| 2000 | C -> T | Yes |
| 2009 | G -> A | Yes |
| 2010 | C -> | No |
| 2203 | G -> | No |
| 2203 | G -> C | No |
| 2204 | C -> | No |
| 2204 | C -> G | No |
| 2235 | G -> | No |
| 2266 | A -> | No |
| 2266 | A -> C | No |
| 2302 | C -> T | No |
| 2357 | C -> T | No |

Variant protein HSTGFB1_P5 (SEQ ID NO:466) according to the present invention has an amino acid sequence; it is encoded by transcript(s) HSTGFB1_T8 (SEQ ID NO:435) and HSTGFB1_T9 (SEQ ID NO:436). An alignment is given to the known protein (Transforming growth factor beta 1 precursor (SEQ ID NO:463)). One or more alignments to one or more previously published protein sequences are given in the alignment table located on the attached CDROM. A brief description of the relationship of the variant protein according to the present invention to each such aligned protein is as follows:

Comparison report between HSTGFB1_P5 (SEQ ID NO:466) and TGFB1_HUMAN (SEQ ID NO:463):

1. An isolated chimeric polypeptide encoding for HSTGFB1_P5 (SEQ ID NO:466), comprising a first amino acid sequence being at least 90% homologous to MPPSGLRLLLLLLPLLWLLVLTPGRPAAGLSTCKTIDMELVKRKRIEAIRGQILSKLRLASPPSQGEV PPGPLPEAVLALYNSTRDRVAGESAEPEPEPEADYYAKEVTRVLMVETHNEIYDKFKQSTHSIYMF FNTSELREAVPEPVLLSRAELRLLRLKLKVEQHVELYQKYSNNSWRYLSNRLLAPSDSPEWLSFDV TGVVRQWLSRGGEIEGFRLSAHCSCDSRDNTLQVDINGFTTGRRGDLATIHGMNRPFLLLMATPLE RAQHLQSSRHRRALDTNYCFSSTEKNCCVRQLYIDFRKDLGWKWIHEPKGYHANFCLGPCPYIWS LDTQYSK corresponding to amino acids 1-338 of TGFB1_HUMAN (SEQ ID NO:463), which also corresponds to amino acids 1-338 of HSTGFB1_P5 (SEQ ID NO:466), and a second amino acid sequence being at least 70%, optionally at least 80%, preferably at least 85%, more preferably at least 90% and most preferably at least 95% homologous to a polypeptide having the sequence LNEQNLIQEVPNIWQREVG (SEQ ID NO: 663) corresponding to amino acids 339-357 of HSTGFB1_P5 (SEQ ID NO:466), wherein said first amino acid sequence and second amino acid sequence are contiguous and in a sequential order.

2. An isolated polypeptide encoding for a tail of HSTGFB1_P5 (SEQ ID NO:466), comprising a polypeptide being at least 70%, optionally at least about 80%, preferably at least about 85%, more preferably at least about 90% and most preferably at least about 95% homologous to the sequence LNEQNLIQEVPNIWQREVG (SEQ ID NO: 663) in HSTGFB1_P5 (SEQ ID NO:466).

The location of the variant protein was determined according to results from a number of different software programs and analyses, including analyses from SignalP and other specialized programs. The variant protein is believed to be located as follows with regard to the cell: secreted.

The glycosylation sites of variant protein HSTGFB1_P5 (SEQ ID NO:466), as compared to the known protein Transforming growth factor beta 1 precursor (SEQ ID NO:463), are described in Table 188 (given according to their position(s) on the amino acid sequence in the first column; the second column indicates whether the glycosylation site is present in the variant protein; and the last column indicates whether the position is different on the variant protein).

TABLE 188
Glycosylation site(s)

| Position(s) on known | Present in | Position in |
|---|---|---|
| amino acid sequence | variant protein? | variant protein? |
| 136 | Yes | 136 |
| 82 | Yes | 82 |
| 176 | Yes | 176 |

The variant protein has the following domains, as determined by using InterPro. The domains are described in Table 189:

TABLE 189
InterPro domain(s)

| InterPro ID | Domain description | Analysis type | Position(s) on protein |
|---|---|---|---|
| IPR003911 | Transforming growth factor | FPrintScan | 163-177, 17-36, 178-193, 195-209, |
| | beta TGFb | | 72-84 |
| IPR003939 | Transforming growth factor, | FPrintScan | 12-31, 135-154, 166-177, 207-219, |
| | beta 1 | | 264-275, 34-43 |
| IPR001839 | Transforming growth factor | HMMPfam | 290-338 |
| | beta | | |
| IPR001111 | Transforming growth factor | HMMPfam | 33-252 |
| | beta (TGFb), N-terminal | | |
| IPR001839 | Transforming growth factor | HMMSmart | 293-357 |
| | beta | | |
| IPR001839 | Transforming growth factor | ScanRegExp | 311-326 |
| | beta | | |
| IPR001839 | Transforming growth factor | BlastProDom | 279-339 |
| | beta | | |
| IPR001839 | Transforming growth factor | ProfileScan | 289-328 |
| | beta | | |
| IPR003911 | Transforming growth factor | FPrintScan | 163-177, 17-36, 178-193, 195-209, |
| | beta TGFb | | 72-84 |
| IPR003939 | Transforming growth factor, | FPrintScan | 12-31, 135-154, 166-177, 207-219, |
| | beta 1 | | 264-275, 34-43 |
| IPR001839 | Transforming growth factor | HMMPfam | 290-338 |
| | beta | | |
| IPR001111 | Transforming growth factor | HMMPfam | 33-252 |
| | beta (TGFb), N-terminal | | |
| IPR001839 | Transforming growth factor | HMMSmart | 293-357 |
| | beta | | |
| IPR001839 | Transforming growth factor | ScanRegExp | 311-326 |
| | beta | | |
| IPR001839 | Transforming growth factor | BlastProDom | 279-339 |
| | beta | | |
| IPR001839 | Transforming growth factor | ProfileScan | 289-328 |
| | beta | | |

Variant protein HSTGFB1_P5 (SEQ ID NO:466) is encoded by the following transcript(s): HSTGFB1_T8 (SEQ ID NO:435) and HSTGFB1_T9 (SEQ ID NO:436).

The coding portion of transcript HSTGFB1_T8 (SEQ ID NO:435) starts at position 1038 and ends at position 2108. The transcript also has the following SNPs as listed in Table 190 (given according to their position on the nucleotide sequence, with the alternative nucleic acid listed; the last column indicates whether the SNP is known or not; the presence of known SNPs in variant protein HSTGFB1_P5 (SEQ ID NO:466) sequence provides support for the deduced sequence of this variant protein according to the present invention).

TABLE 190
Nucleic acid SNPs

| SNP position on | Alternative | Previously |
|---|---|---|
| nucleotide sequence | nucleic acid | known SNP? |
| 181 | C -> A | Yes |
| 205 | C -> T | Yes |
| 206 | T -> A | Yes |
| 211 | G -> C | Yes |
| 214 | G -> | No |
| 289 | C -> | No |
| 390 | G -> C | Yes |
| 585 | G -> A | Yes |
| 651 | C -> T | Yes |
| 762 | C -> A | Yes |
| 789 | C -> A | Yes |
| 896 | C -> T | Yes |
| 1024 | G -> A | Yes |
| 1030 | C -> | No |
| 1030 | C -> T | No |
| 1066 | T -> C | Yes |
| 1111 | G -> C | Yes |
| 1177 | A -> G | Yes |
| 1232 | G -> | No |
| 1443 | A -> C | No |
| 1725 | A -> | No |
| 1725 | A -> T | No |
| 1825 | C -> T | Yes |
| 2000 | C -> T | Yes |
| 2009 | G -> A | Yes |
| 2010 | C -> | No |

The coding portion of transcript HSTGFB1_T9 (SEQ ID NO:436) starts at position 1038 and ends at position 2108. The transcript also has the following SNPs as listed in Table 191 (given according to their position on the nucleotide sequence, with the alternative nucleic acid listed; the last column indicates whether the SNP is known or not; the presence of known SNPs in variant protein HSTGFB1_P5 (SEQ ID NO:466) sequence provides support for the deduced sequence of this variant protein according to the present invention).

TABLE 191
Nucleic acid SNPs

| SNP position on | Alternative | Previously |
|---|---|---|
| nucleotide sequence | nucleic acid | known SNP? |
| 181 | C -> A | Yes |
| 205 | C -> T | Yes |
| 206 | T -> A | Yes |
| 211 | G -> C | Yes |
| 214 | G -> | No |
| 289 | C -> | No |
| 390 | G -> C | Yes |
| 585 | G -> A | Yes |
| 651 | C -> T | Yes |
| 762 | C -> A | Yes |
| 789 | C -> A | Yes |
| 896 | C -> T | Yes |
| 1024 | G -> A | Yes |
| 1030 | C -> | No |
| 1030 | C -> T | No |
| 1066 | T -> C | Yes |
| 1111 | G -> C | Yes |
| 1177 | A -> G | Yes |
| 1232 | G -> | No |
| 1443 | A -> C | No |
| 1725 | A -> | No |
| 1725 | A -> T | No |
| 1825 | C -> T | Yes |
| 2000 | C -> T | Yes |
| 2009 | G -> A | Yes |
| 2010 | C -> | No |
| 2245 | T -> A | Yes |
| 2428 | C -> T | Yes |

Variant protein HSTGFB1_P7 (SEQ ID NO:467) according to the present invention has an amino acid sequence; it is encoded by transcript(s) HSTGFB1_T11 (SEQ ID NO:437). An alignment is given to the known protein (Transforming growth factor beta 1 precursor (SEQ ID NO:463)). One or more alignments to one or more previously published protein sequences are given in the alignment table located on the attached CDROM. A brief description of the relationship of the variant protein according to the present invention to each such aligned protein is as follows:

Comparison report between HSTGFB1_P7 (SEQ ID NO:467) and TGFB1_HUMAN (SEQ ID NO:463):

1. An isolated chimeric polypeptide encoding for HSTGFB1_P7 (SEQ ID NO:467), comprising a first amino acid sequence being at least 90% homologous to MPPSGLRLLLLLLPLLWLLVLTPGRPAAGLSTCKTIDMELVKRKRIEAIRGQILSKLRLASPPSQGEV PPGPLPEAVLALYNSTRDRVAGESAEPEPEPEADYYAKEVTRVLMVETHNEIYDKFKQSTHSIYMF FNTSELREAVPEPVLLSRAELRLLRLKLKVEQHVELYQKYSNNSWRYLSNRLLAPSDSPEWLSFDV TGVVRQWLSRGGEIEGFRLSAHCSCDSRDNTLQVDIN corresponding to amino acids 1-237 of TGFB1_HUMAN (SEQ ID NO:463), which also corresponds to amino acids 1-237 of HSTGFB1_P7 (SEQ ID NO:467), and a second amino acid sequence being at least 70%, optionally at least 80%, preferably at least 85%, more preferably at least 90% and most preferably at least 95% homologous to a polypeptide having the sequence APRRRTAACGSCTLTSARTSAGSGSTSPRATMPTSASGPAPTFGAWTRSTARSWPCTTSITRAPRRR RAACRRRWSRCPSCTTWAASPRWSSCPT (SEQ ID NO: 664) corresponding to amino acids 238-332 of HSTGFB1_P7 (SEQ ID NO:467), wherein said first amino acid sequence and second amino acid sequence are contiguous and in a sequential order.

2. An isolated polypeptide encoding for a tail of HSTGFB1_P7 (SEQ ID NO:467), comprising a polypeptide being at least 70%, optionally at least about 80%, preferably at least about 85%, more preferably at least about 90% and most preferably at least about 95% homologous to the sequence APRRRTAACGSCTLTSARTSAGSGSTSPRATMPTSASGPAPTFGAWTRSTARSWPCTTSITRAPRRR RAACRRRWSRCPSCTTWAASPRWSSCPT (SEQ ID NO: 664) in HSTGFB1_P7 (SEQ ID NO:467).

The location of the variant protein was determined according to results from a number of different software programs and analyses, including analyses from SignalP and other specialized programs. The variant protein is believed to be located as follows with regard to the cell: secreted.

The glycosylation sites of variant protein HSTGFB1_P7 (SEQ ID NO:467), as compared to the known protein Transforming growth factor beta 1 precursor (SEQ ID NO:463), are described in Table 192 (given according to their position(s) on the amino acid sequence in the first column; the second column indicates whether the glycosylation site is present in the variant protein; and the last column indicates whether the position is different on the variant protein).

TABLE 192
Glycosylation site(s)

| Position(s) on known | Present in | Position in |
|---|---|---|
| amino acid sequence | variant protein? | variant protein? |
| 136 | yes | 136 |
| 82 | yes | 82 |
| 176 | yes | 176 |

The variant protein has the following domains, as determined by using InterPro. The domains are described in Table 193:

TABLE 193
InterPro domain(s)

| | Domain | | |
|---|---|---|---|
| InterPro ID | description | Analysis type | Position(s) on protein |
| IPR003911 | Transforming | FPrintScan | 163-177, 17-36, |
| | growth factor | | 178-193, 195-209, |
| | beta TGFb | | 72-84 |
| IPR003939 | Transforming | FPrintScan | 12-31, 135-154, |
| | growth factor, | | 166-177, 207-219, |
| | beta 1 | | 34-43 |
| IPR001111 | Transforming | HMMPfam | 33-236 |
| | growth factor | | |
| | beta (TGFb), | | |
| | N-terminal | | |

Variant protein HSTGFB1_P7 (SEQ ID NO:467) is encoded by the following transcript(s): HSTGFB1_T11 (SEQ ID NO:437). The coding portion of transcript HSTGFB1_T11 (SEQ ID NO:437) starts at position 1038 and ends at position 2033. The transcript also has the following SNPs as listed in Table 194 (given according to their position on the nucleotide sequence, with the alternative nucleic acid listed; the last column indicates whether the SNP is known or not; the presence of known SNPs in variant protein HSTGFB1_P7 (SEQ ID NO:467) sequence provides support for the deduced sequence of this variant protein according to the present invention).

TABLE 194
Nucleic acid SNPs

| SNP position on | Alternative | Previously |
|---|---|---|
| nucleotide sequence | nucleic acid | known SNP? |
| 181 | C -> A | Yes |
| 205 | C -> T | Yes |
| 206 | T -> A | Yes |
| 211 | G -> C | Yes |
| 214 | G -> | No |
| 289 | C -> | No |
| 390 | G -> C | Yes |
| 585 | G -> A | Yes |
| 651 | C -> T | Yes |
| 762 | C -> A | Yes |
| 789 | C -> A | Yes |
| 896 | C -> T | Yes |
| 1024 | G -> A | Yes |
| 1030 | C -> | No |
| 1030 | C -> T | No |
| 1066 | T -> C | Yes |
| 1111 | G -> C | Yes |
| 1177 | A -> G | Yes |
| 1232 | G -> | No |
| 1443 | A -> C | No |
| 1725 | A -> | No |
| 1725 | A -> T | No |
| 1852 | C -> T | Yes |
| 1861 | G -> A | Yes |
| 1862 | C -> | No |
| 1967 | G -> | No |
| 1967 | G -> C | No |
| 1968 | C -> | No |
| 1968 | C -> G | No |
| 1999 | G -> | No |
| 2030 | A -> | No |
| 2030 | A -> C | No |
| 2066 | C -> T | No |
| 2121 | C -> T | No |

Variant protein HSTGFB1_P10 (SEQ ID NO:468) according to the present invention has an amino acid sequence; it is encoded by transcript(s) HSTGFB1_T14 (SEQ ID NO:438). The location of the variant protein was determined according to results from a number of different software programs and analyses, including analyses from SignalP and other specialized programs. The variant protein is believed to be located as follows with regard to the cell: secreted.

The variant protein has the following domains, as determined by using InterPro. The domains are described in Table 195:

TABLE 195
InterPro domain(s)

| InterPro ID | Domain description | Analysis type | Position(s) on protein |
|---|---|---|---|
| IPR003911 | Transforming growth factor | FPrintScan | 163-177, 17-36, 178-193, 195-209, |
| | beta TGFb | | 72-84 |
| IPR003939 | Transforming growth factor, | FPrintScan | 12-31, 135-154, 166-177, 207-219, |
| | beta 1 | | 34-43 |
| IPR001839 | Transforming growth factor | HMMPfam | 319-370 |
| | beta | | |
| IPR001111 | Transforming growth factor | HMMPfam | 33-236 |
| | beta (TGFb), N-terminal | | |
| IPR001839 | Transforming growth factor | HMMSmart | 311-370 |
| | beta | | |
| IPR001839 | Transforming growth factor | BlastProDom | 318-370 |
| | beta | | |
| IPR001839 | Transforming growth factor | ProfileScan | 319-370 |
| | beta | | |

Variant protein HSTGFB1_P10 (SEQ ID NO:468) is encoded by the following transcript(s): HSTGFB1_T14 (SEQ ID NO:438). The coding portion of transcript HSTGFB1_T14 (SEQ ID NO:438) starts at position 1038 and ends at position 2147. The transcript also has the following SNPs as listed in Table 196 (given according to their position on the nucleotide sequence, with the alternative nucleic acid listed; the last column indicates whether the SNP is known or not; the presence of known SNPs in variant protein HSTGFB1_P10 (SEQ ID NO:468) sequence provides support for the deduced sequence of this variant protein according to the present invention).

TABLE 196
Nucleic acid SNPs

| SNP position on | Alternative | Previously |
|---|---|---|
| nucleotide sequence | nucleic acid | known SNP? |
| 181 | C -> A | Yes |
| 205 | C -> T | Yes |
| 206 | T -> A | Yes |
| 211 | G -> C | Yes |
| 214 | G -> | No |
| 289 | C -> | No |
| 390 | G -> C | Yes |
| 585 | G -> A | Yes |
| 651 | C -> T | Yes |
| 762 | C -> A | Yes |
| 789 | C -> A | Yes |
| 896 | C -> T | Yes |
| 1024 | G -> A | Yes |
| 1030 | C -> | No |
| 1030 | C -> T | No |
| 1066 | T -> C | Yes |
| 1111 | G -> C | Yes |
| 1177 | A -> G | Yes |
| 1232 | G -> | No |
| 1443 | A -> C | No |
| 1725 | A -> | No |
| 1725 | A -> T | No |
| 1852 | C -> T | Yes |
| 1861 | G -> A | Yes |
| 1862 | C -> | No |
| 2055 | G -> | No |
| 2055 | G -> C | No |
| 2056 | C -> | No |
| 2056 | C -> G | No |
| 2087 | G -> | No |
| 2118 | A -> | No |
| 2118 | A -> C | No |
| 2154 | C -> T | No |
| 2209 | C -> T | No |

Table 197 below describes the starting and ending position of HSTGFB1_node_{—}7 (SEQ ID NO:443) on the relevant ranscripts. Experimental results for this segment are described below.

TABLE 197
Segment location on transcripts

| | Segment | Segment |
|---|---|---|
| Transcript name | starting position | ending position |
| HSTGFB1_T5 (SEQ ID NO: 433) | 1220 | 1392 |
| HSTGFB1_T6 (SEQ ID NO: 434) | 1220 | 1392 |
| HSTGFB1_T8 (SEQ ID NO: 435) | 1220 | 1392 |
| HSTGFB1_T9 (SEQ ID NO: 436) | 1220 | 1392 |
| HSTGFB1_T11 (SEQ ID NO: 437) | 1220 | 1392 |
| HSTGFB1_T14 (SEQ ID NO: 438) | 1220 | 1392 |

Table 198 below describes the starting and ending position of HSTGFB1_node_{—}15 (SEQ ID NO:445) on the relevant transcript. Experimental results for this segment are described below.

TABLE 198
Segment location on transcripts

| | Segment | Segment |
|---|---|---|
| Transcript name | starting position | ending position |
| HSTGFB1_T5 (SEQ ID NO: 433) | 1750 | 1888 |

Table 199 below describes the starting and ending position of HSTGFB1_node_{—}14 (SEQ ID NO:454) on the relevant ranscripts. Experimental results for this segment are described below.

TABLE 199
Segment location on transcripts

| | Segment | Segment |
|---|---|---|
| Transcript name | starting position | ending position |
| HSTGFB1_T5 (SEQ ID NO: 433) | 1672 | 1749 |
| HSTGFB1_T6 (SEQ ID NO: 434) | 1672 | 1749 |
| HSTGFB1_T8 (SEQ ID NO: 435) | 1672 | 1749 |
| HSTGFB1_T9 (SEQ ID NO: 436) | 1672 | 1749 |
| HSTGFB1_T11 (SEQ ID NO: 437) | 1672 | 1749 |
| HSTGFB1_T14 (SEQ ID NO: 438) | 1672 | 1749 |

Expression of transforming growth factor, beta 1 HSTGFB1 transcripts which are detectable by amplicon as depicted in sequence name HSTGFB1seg14-15 (SEQ ID NO: 471) in normal and cancerous colon, breast, ovary and lung tissues:

Expression of transforming growth factor, beta 1 transcripts detectable by or according to seg14-15, HSTGFB1seg14-15 (SEQ ID NO: 471) amplicon and primers HSTGFB1seg14-15F (SEQ ID NO: 469) and HSTGFB1seg14-15R (SEQ ID NO: 470) was measured by real time PCR. In parallel the expression of four housekeeping genes was measured similarly. Four different cancer panels were checked with different combinations of house keeping genes:

Colon panel was checked with —PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), RPS27A (GenBank Accession No. NM_{—}002954 (SEQ ID NO:1); RPS27A amplicon (SEQ ID NO: 47)) and G6PD (GenBank Accession No. NM_{—}000402 (SEQ ID NO:8); G6PD amplicon (SEQ ID NO: 44))

Breast panel was checked with PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and G6PD (GenBank Accession No. NM_{—}000402 (SEQ ID NO:8); G6PD amplicon (SEQ ID NO: 44))

Lung panel was checked with PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and Ubiquitin (GenBank Accession No. BC000449 (SEQ ID NO:9); amplicon—Ubiquitin-amplicon (SEQ ID NO:50)).

Ovary panel was checked with PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41))

For each RT sample, the expression of the above amplicon was normalized to the geometric mean of the quantities of the housekeeping genes. The normalized quantity of each RT sample was then divided by the median of the quantities of the normal post-mortem (PM) samples within each panel (Sample Nos. 41, 52, 62-67, 69-71 in colon, 56-60, 63-67 in breast, Sample Nos. 45, 46, 48, 71 in ovary and 47-50, 90-93, 96-99 in lung; “Tissue samples in ovarian cancer testing panel” table 3 above, “Tissue samples in colon cancer testing panel” table 4 above, “Tissue samples in lung cancer testing panel” table 5 above, “Tissue samples in breast cancer testing panel” table 6 above), to obtain a value of fold differential expression for each sample relative to median of the normal PM samples.

In one experiment that was carried out with each of the above panels, no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: HSTGFB1seg14-15F (SEQ ID NO: 469) forward primer; and HSTGFB1seg 14-15R (SEQ ID NO: 470) reverse primer.

The present invention also preferably encompasses any amplicon obtained through the use of any suitable primer pair; for example, for the above experiment, the following amplicon was obtained as a non-limiting illustrative example only of a suitable amplicon: HSTGFB1seg14-15 (SEQ ID NO: 471).

Primers:

Forward primer HSTGFB1seg14-15F (SEQ ID NO:469):
TGAGGGCTTTCGCCTTAGC
Reverse primer HSTGFB1seg14-15R (SEQ ID NO:470):
CACACGTCACAACTGGGCAT
Amplicon HSTGFB1seg14-15 (SEQ ID NO:471):
TGAGGGCTTTCGCCTTAGCGCCCACTGCTCCTGTGACAGCAGGGATAACA
CACTGCAAGTGGACATCAACGGTGAGGCCTGCTTCCCCGGCCATGCCCAG
TTGTGACGTGTG

Expression of transforming growth factor, beta 1 HSTGFB1 transcripts which are detectable by amplicon as depicted in sequence name HSTGFB1 junc14-22-23 (SEQ ID NO: 474) in normal and cancerous colon, breast, ovary and lung tissues

Expression of transforming growth factor, beta 1 transcripts detectable by or according to HSTGFB1 junc14-22-23 (SEQ ID NO: 474) amplicon and primers HSTGFB1 junc14-22-23F (SEQ ID NO: 472) and HSTGFB1 junc14-22-23R (SEQ ID NO: 473) was measured by real time PCR. In parallel the expression of four housekeeping genes was measured similarly. Four different cancer panels were checked with different combinations of house keeping genes:

Colon panel was checked with —PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), RPS27A (GenBank Accession No. NM_{—}002954 (SEQ ID NO:1); RPS27A amplicon (SEQ ID NO: 47)) and G6PD (GenBank Accession No. NM_{—}000402 (SEQ ID NO:8); G6PD amplicon (SEQ ID NO: 44))

Breast panel was checked with PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and G6PD (GenBank Accession No. NM_{—}000402 (SEQ ID NO:8); G6PD amplicon (SEQ ID NO: 44))

Lung panel was checked with PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and Ubiquitin (GenBank Accession No. BC000449 (SEQ ID NO:9); amplicon—Ubiquitin-amplicon (SEQ ID NO:50)).

Ovary panel was checked with PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41))

For each RT sample, the expression of the above amplicon was normalized to the geometric mean of the quantities of the housekeeping genes. The normalized quantity of each RT sample was then divided by the median of the quantities of the normal post-mortem (PM) samples within each panel (Sample Nos. 41, 52, 62-67, 69-71 in colon, 56-60, 63-67 in breast, Sample Nos. 45, 46, 48, 71 in ovary and 47-50, 90-93, 96-99 in lung; Tables 4, 6, 3, 5 above, “Tissue samples in ovarian cancer testing panel”, “Tissue samples in colon cancer testing panel”, “Tissue samples in lung cancer testing panel”, “Tissue samples in breast cancer testing panel”, respectively), to obtain a value of fold differential expression for each sample relative to median of the normal PM samples.

In one experiment that was carried out with each of the above panels, no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: HSTGFB1 junc14-22-23F (SEQ ID NO: 472) forward primer; and HSTGFB1 junc14-22-23R (SEQ ID NO: 473) reverse primer.

The present invention also preferably encompasses any amplicon obtained through the use of any suitable primer pair; for example, for the above experiment, the following amplicon was obtained as a non-limiting illustrative example only of a suitable amplicon: HSTGFB1 junc14-22-23 (SEQ ID NO: 474).

Primers:

Forward primer HSTGFB1junc14-22-23F
(SEQ ID NO:472):
CAAGTGGACATCAACGCTCCA
Reverse primer HSTGFB1junc14-22-23R
(SEQ ID NO:473):
GGGCCAGACGTACCTTGCT
Amplicon HSTGFB1 junc14-22-23 (SEQ ID NO:474):
CAAGTGGACATCAACGCTCCACGGAGAAGAACTGCTGCGTGCGGCAGCTG
TACATTGACTTCCGCAAGGACCTCGGCTGGAAGTGGATCCACGAGCCCAA
GGGCTACCATGCCAACTTCTGCCTCGGGCCCTGCCCCTACATTTGGAGCC
TGGACACGCAGTACAGCAAGGTACGTCTGGCCC

Expression of transforming growth factor, beta 1 HSTGFB1 transcripts which are detectable by amplicon as depicted in sequence name HSTGFB1 seg7WT (SEQ ID NO:477) in normal and cancerous colon, breast, ovary and lung tissues

Expression of transforming growth factor, beta 1 transcripts detectable by or according to HSTGFB1 seg7WT (SEQ ID NO:477) amplicon and primers HSTGFB1 seg7WT-F (SEQ ID NO:475) and HSTGFB1 seg7WT-R (SEQ ID NO:476) was measured by real time PCR. In parallel the expression of four housekeeping genes was measured similarly. Four different cancer panels were checked with different combinations of house keeping genes:

Colon panel was checked with —PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), RPS27A (GenBank Accession No. NM_{—}002954 (SEQ ID NO:1); RPS27A amplicon (SEQ ID NO: 47)) and G6PD (GenBank Accession No. NM_{—}000402 (SEQ ID NO:8); G6PD amplicon (SEQ ID NO: 44))

Breast panel was checked with PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and G6PD (GenBank Accession No. NM_{—}000402 (SEQ ID NO:8); G6PD amplicon (SEQ ID NO: 44))

Lung panel was checked with PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and Ubiquitin (GenBank Accession No. BC000449 (SEQ ID NO:9); amplicon—Ubiquitin-amplicon (SEQ ID NO:50)).

Ovary panel was checked with PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41))

For each RT sample, the expression of the above amplicon was normalized to the geometric mean of the quantities of the housekeeping genes. The normalized quantity of each RT sample was then divided by the median of the quantities of the normal post-mortem (PM) samples within each panel (Sample Nos. 41, 52, 62-67, 69-71 in colon, 56-60, 63-67 in breast, Sample Nos. 45, 46, 48, 71 in ovary and 47-50, 90-93, 96-99 in lung; Tables 4, 6, 3, 5 above, “Tissue samples in ovarian cancer testing panel”, “Tissue samples in colon cancer testing panel”, “Tissue samples in lung cancer testing panel”, “Tissue samples in breast cancer testing panel”, respectively), to obtain a value of fold differential expression for each sample relative to median of the normal PM samples.

In one experiment that was carried out with each of the above panels, no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: HSTGFB1 seg7F (SEQ ID NO: 475) forward primer; and HSTGFB1 seg7R (SEQ ID NO: 476) reverse primer.

The present invention also preferably encompasses any amplicon obtained through the use of any suitable primer pair; for example, for the above experiment, the following amplicon was obtained as a non-limiting illustrative example only of a suitable amplicon: HSTGFB1 seg7 (SEQ ID NO: 477).

Primers:

Forward primer HSTGFB1 seg7F (SEQ ID NO:475):
GCTCGCCCTGTACAACAGC
Reverse primer HSTGFB1 seg7R (SEQ ID NO:476):
GACCTCCTTGGCGTAGTAGTCG
Amplicon HSTGFB1 seg7 (SEQ ID NO:477):
GCTCGCCCTGTACAACAGCACCCGCGACCGGGTGGCCGGGGAGAGTGCAG
AACCGGAGCCCGAGCCTGAGGCCGACTACTACGCCAAGGAGGTC

Expression of transforming growth factor, beta 1 (HSTGFB1) transcripts which are detectable by amplicon as depicted in sequence name HSTGFB1junc14-22-23 (SEQ ID NO: 474), HSTGFB1seg14-15 (SEQ ID NO: 471) and HSTGFB1 seg7WT (SEQ ID NO:477) in different normal tissues

Expression of transforming growth factor, beta 1 transcripts detectable by or according to HSTGFB1junc14-22-23 (SEQ ID NO: 474), HSTGFB1seg14-15 (SEQ ID NO: 471) and HSTGFB1 seg7WT (SEQ ID NO:477) amplicons and primers: HSTGFB1junc14-22-23F (SEQ ID NO: 472), HSTGFB1junc14-22-23R (SEQ ID NO: 473), HSTGFB1seg14-15F (SEQ ID NO: 469), HSTGFB1seg14-15R (SEQ ID NO: 470), HSTGFB1 seg7WT-F (SEQ ID NO:475) and HSTGFB1 seg7WT-R (SEQ ID NO:476) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicon was normalized to the geometric mean of the quantities of the housekeeping genes. For each amplicon, the normalized quantity of each RT sample was then divided by the median of the quantities of the lung samples (Sample Nos. 15-17, Table 7, “Tissue samples in normal panel”), to obtain a value of relative expression of each sample relative to median of the lung samples.

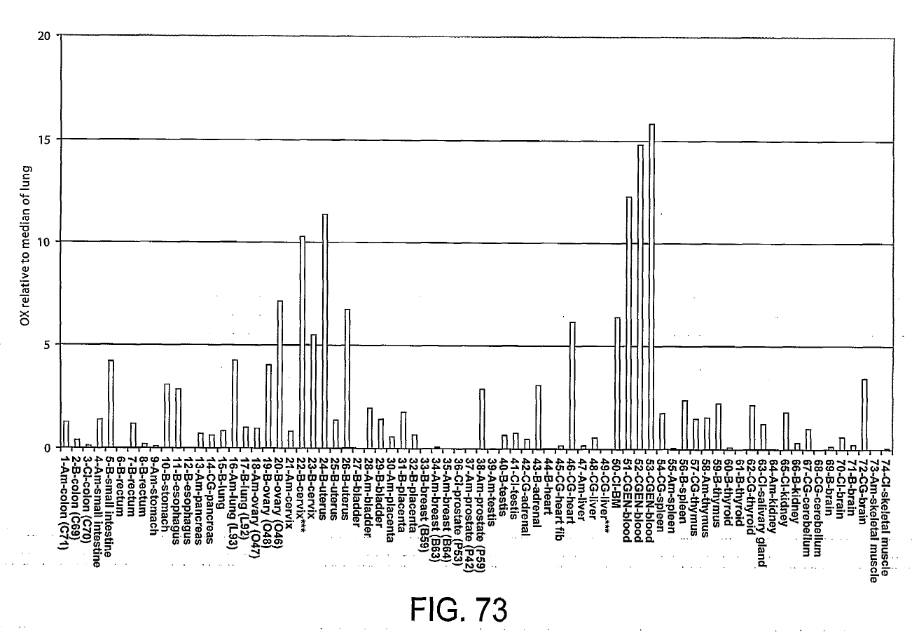

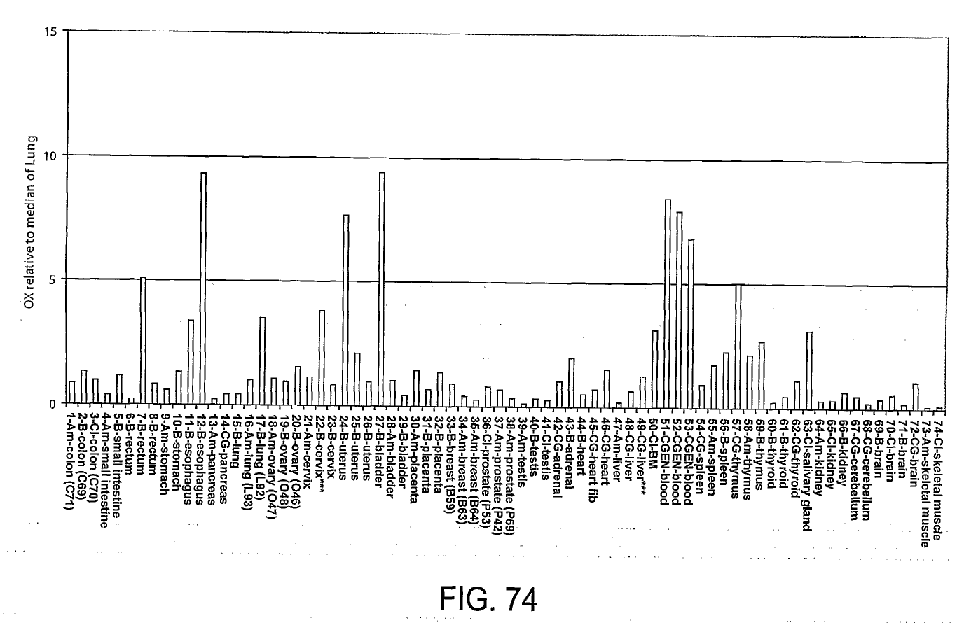

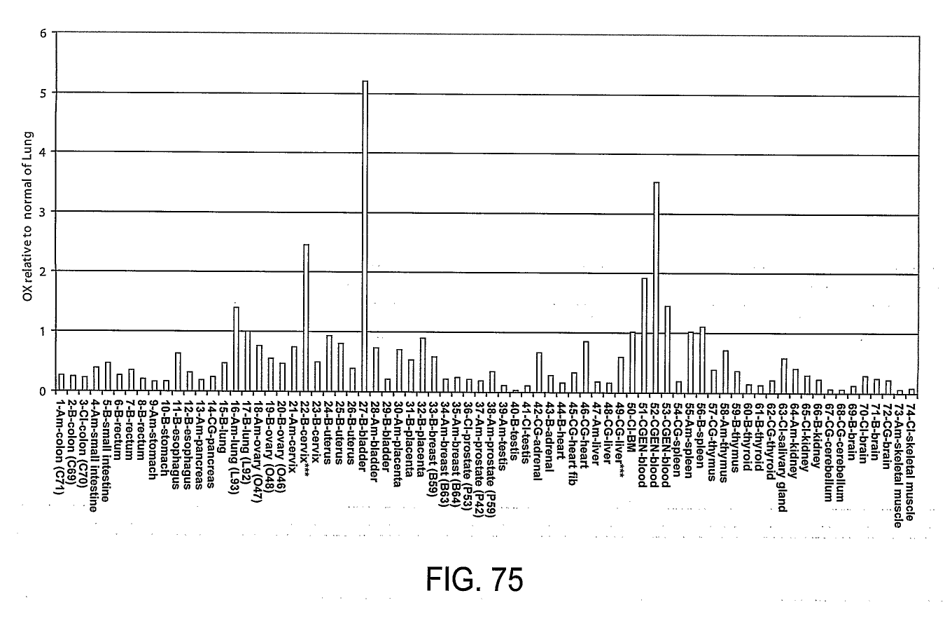

FIG. 73 is a histogram showing Expression of transforming growth factor, beta 1 (HSTGFB1) transcripts which are detectable by amplicon as depicted in sequence name HSTGFB1junc14-22-23 (SEQ ID NO: 474) in different normal tissues. FIG. 74 is a histogram showing Expression of transforming growth factor, beta 1 (HSTGFB1) transcripts which are detectable by amplicon as depicted in sequence name HSTGFB1seg14-15 (SEQ ID NO: 471) in different normal tissues. FIG. 75 is a histogram showing Expression of transforming growth factor, beta 1 (HSTGFB1) transcripts which are detectable by amplicon as depicted in sequence name HSTGFB1 seg7WT (SEQ ID NO:477) in different normal tissues.

HSTGFB1junc14-22-23 Primers and Amplicon:

Forward primer HSTGFB1junc14-22-23F
(SEQ ID NO:472):
CAAGTGGACATCAACGCTCCA
Reverse primer HSTGFB1junc14-22-23R
(SEQ ID NO:473):
GGGCCAGACGTACCTTGCT
Amplicon HSTGFB1junc14-22-23 (SEQ ID NO:474):
CAAGTGGACATCAACGCTCCACGGAGAAGAACTGCTGCGTGCGGCAGCTG
TACATTGACTTCCGCAAGGACCTCGGCTGGAAGTGGATCCACGAGCCCAA
GGGCTACCATGCCAACTTCTGCCTCGGGCCCTGCCCCTACATTTGGAGCC
TGGACACGCAGTACAGCAAGGTACGTCTGGCCC

HSTGFB1seg14-15 Primers and Amplicon:

Forward primer HSTGFB1seg14-15F (SEQ ID NO:469):
TGAGGGCTTTCGCCTTAGC
Reverse primer HSTGFB1seg14-15R (SEQ ID NO:470):
CACACGTCACAACTGGGCAT
Amplicon HSTGFB1seg14-15 (SEQ ID NO:471):
TGAGGGCTTTCGCCTTAGCGCCCACTGCTCCTGTGACAGCAGGGATAACA
CACTGCAAGTGGACATCAACGGTGAGGCCTGCTTCCCCGGCCATGCCCAG
TTGTGACGTGTG

HSTGFB1seg7-WT Primers and Amplicon:

Forward primer HSTGFB1 seg7F (SEQ ID NO:475):
GCTCGCCCTGTACAACAGC
Reverse primer HSTGFB1 seg7R (SEQ ID NO:476):
GACCTCCTTGGCGTAGTAGTCG
Amplicon HSTGFB1 seg7 (SEQ ID NO:477):
GCTCGCCCTGTACAACAGCACCCGCGACCGGGTGGCCGGGGAGAGTGCAG
AACCGGAGCCCGAGCCTGAGGCCGACTACTACGCCAAGGAGGTC

Expression of transforming growth factor, beta 1 transcripts which are detectable by segment 15, in normal and cancerous breast tissues

Expression of transforming growth factor, beta 1 transcripts detectable by or according to segment 15 was measured with oligonucleotide-based micro-arrays. The results of image intensities for each feature were normalized according to the percentile 95 of the image intensities of all the features on the chip. Then, feature image intensities for replicates of the same oligonucleotide on the chip and replicates of the same sample were averaged. Outlying results were discarded.

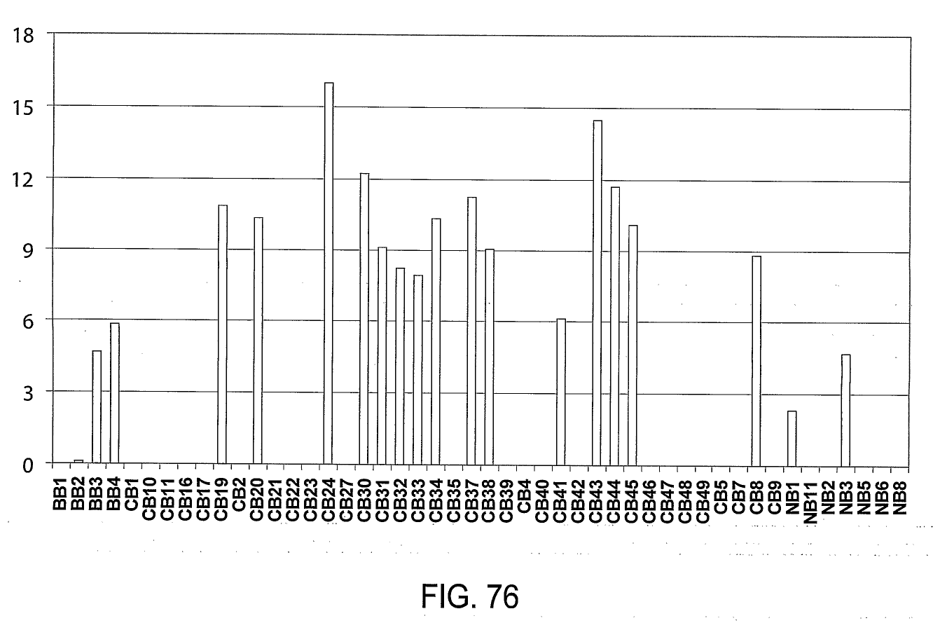

For oligonucleotide HSTGFB1_{—}0_{—}9_{—}56 (SEQ ID NO:13) the averaged intensity determined for every sample was divided by the averaged intensity of all the normal samples (Samples NB 1-3, 5, 6, 8, 11, Table 200, below), to obtain a value of fold up-regulation for each sample relative to the averaged normal samples. These data are presented in a histogram shown in FIG. 76. As is evident from the histogram, the expression of transforming growth factor, beta 1 transcripts detectable with the above oligonucleotides in cancer samples was significantly higher than in the normal samples.

>HSTGFB1_0_9_56 (SEQ ID NO:13)
GTTCCCATCTGCCCCACGCCCCACTTATCTATCCCTCTGAGAGTGTGTGT

TABLE 200

| | | | | | | | | | |
|---|---|---|---|---|---|---|---|---|---|
| Sample | MA- | | | | | | | stage | |
| RT# | TAA | Sample rename | Lot no | source | pathology | age | grade | (TNM) | stage |

Cancer

| | | | | | | | | | |
|---|---|---|---|---|---|---|---|---|---|
| RT-1 | BC-1 | 14-A-IDC G2 | A0135T | ABS | IDC | 37 | 2 | T2N2Mx | |
| RT-2 | BC-2 | 43-B-IDC G2 | A609183 | Biochain | IDC | 40 | 2 | | |
| RT-3 | BC-3 | 54-B-IDC G2 | A605353 | Biochain | IDC | 41 | 2 | | |
| RT-4 | BC-4 | 55-B-IDC G2 | A609179 | Biochain | IDC | 42 | 2 | | |
| RT-5 | BC-5 | 17-A-IDC G2 | 4904020036T | ABS | IDC | 42 | 2-3 | T3N1Mx | |
| RT-6 | BC-6 | 42-A-IDC G3 | 6005020031T | ABS | IDC | 42 | 3 | T1cN0Mx | |
| RT-7 | BC-7 | 7-A-IDC G2 | 7263T | ABS | IDC | 43 | 2 | T1N0M0 | stage 1 |
| RT-8 | BC-8 | 45-B-IDC G2 | A609222 | Biochain | IDC | 44 | 2 | | |
| RT-9 | BC-9 | 12-A-IDC G2 | 1432T | ABS | IDC | 46 | 2 | T2N0M0 | stage |
| | | | | | | | | | 2A |
| RT-10 | BC-10 | 46-B-Carci G2 | A609177 | Biochain | Carcinoma | 48 | 2 | | |
| RT-11 | BC-11 | 16-A-IDC G2 | 4904020032T | ABS | IDC | 49 | 2 | T3N1Mx | |
| RT-12 | BC-12 | 49-B-IDC G2 | A609223 | Biochain | IDC | 54 | 2 | | |
| RT-13 | BC-13 | 32-A-Muc | 7116T | ABS | Mucinous | 54 | | T2N0M0 | stage |
| | | Carci | | | | | | | 2A |
| RT-14 | BC-14 | 45-B-IDC G2 | A609181 | Biochain | IDC | 58 | 2 | | |
| RT-15 | BC-15 | 15-A-IDC G2 | 7259T | ABS | IDC | 59 | 2 | T3N1M0 | stage |
| | | | | | | | | | 3A |
| RT-T6 | BC-16 | 6-A-IDC G1 | 7238T | ABS | IDC | 60 | 1 | T2N0M0 | stage |
| | | | | | | | | | 2A |
| RT-17 | BC-17 | 26-A-IDC G3 | 7249T | ABS | IDC | 60 | 3 | T2N0M0 | stage |
| | | | | | | | | | 2A |
| RT-18 | BC-18 | 13-A-IDC G2 | A0133T | ABS | IDC | 63 | 2 | T2N1aMx | |
| RT-19 | BC-19 | 50-B-IDC G2 | A609224 | Biochain | IDC | 69 | 2 | | |
| RT-20 | BC-20 | 44-B-IDC G2 | A609198 | Biochain | IDC | 77 | 2 | | |
| RT-21 | BC-21 | 51-B-IDC G1 | A605361 | Biochain | IDC | 79 | 1 | | |
| RT-22 | BC-22 | 27-A-IDC G3 | 4907020072T | ABS | IDC | 91 | 3 | T2N0Mx | |
| RT-23 | BC-23 | 3Z5Z4ANH | 3Z5Z4RVE | GCI | IDC | 60 | | | stage |
| | | | | | | | | | 2B |
| RT-24 | BC-24 | 4W2NYAC1 | 4W2NYR9S | GCI | IDC | 39 | | | stage |
| | | | | | | | | | 3B |
| RT-25 | BC-25 | 54NTAAKT | 54NTAR75 | GCI | IDC | 67 | | | stage |
| | | | | | | | | | 2B |
| RT-26 | BC-26 | I2YLEACP | I2YLERVY | GCI | IDC | 60 | | | stage 1 |
| RT-27 | BC-27 | J5MPNA9Q | J5MPNRQI | GCI | IDC | 64 | | | stage |
| | | | | | | | | | 2B |
| RT-28 | BC-28 | KIOE7AI9 | KIOE7RWK | GCI | IDC | 65 | | | stage |
| | | | | | | | | | 3B |
| RT-29 | BC-29 | OLKL4AO6 | OLKL4RZ9 | GCI | IDC | 46 | | | stage |
| | | | | | | | | | 2B |
| RT-30 | BC-30 | RD3F9AFQ | RD3F9RY9 | GCI | IDC | 41 | | | stage |
| | | | | | | | | | 3A |
| RT-31 | BC-31 | SE5BKAEQ | SE5BKRHY | GCI | IDC | 41 | | | stage |
| | | | | | | | | | 2B |
| RT-32 | BC-32 | VK1EJAQE | VK1EJRKH | GCI | IDC | 54 | | | stage 2 |
| RT-33 | BC-33 | YOLOFARG | YOLOFRE7 | GCI | IDC | 62 | | | stage |
| | | | | | | | | | 3A |
| RT-34 | BC-34 | YQ1WWAUV | YQ1WWROR | GCI | IDC | 62 | | | stage |
| | | | | | | | | | 3B |
| RT-35 | BC-35 | YSZ67A48 | YSZ67ROA | GCI | IDC | 46 | | | stage |
| | | | | | | | | | 2A |
| RT-36 | BC-36 | POPHPAZ4 | POPHPRDM | GCI | IDC | 57 | | | stage 1 |
| RT-37 | BC-37 | 5IRTKAXT | 5IRTKRTG | GCI | IDC | 39 | | | stage 1 |
| RT-38 | BC-38 | DSI52AH3 | DSI52RVW | GCI | IDC | 50 | | | stage 1 |
| RT-39 | BC-39 | GETCVAY2 | GETCVRIT | GCI | IDC | 70 | | | stage |
| | | | | | | | | | 2A |
| RT-40 | BC-40 | S2GBYAGC | S2GBYRR1 | GCI | IDC | 56 | | | stage 1 |
| RT-41 | BC-41 | UT3SEAQY | UT3SERM8 | GCI | IDC | 67 | | | stage |
| | | | | | | | | | 2A |
| RT-42 | BC-42 | PVSYXA72 | PVSYXR66 | GCI | IDC | 70 | | | stage |
| | | | | | | | | | 2A |
| RT-43 | BC-43 | 17138 | 30697A1 | Asterand | IDC | 46 | 3 | T2NXM0 | stage |
| | | | | | | | | | 2A |
| RT-44 | BC-44 | 17959 | 31225A1 | Asterand | IDC | 70 | 2 | T1NXM0 | stage 1 |
| RT-45 | BC-45 | 52-B-ILC G1 | A605360 | Biochain | ILC | 60 | 1 | | |
| RT-46 | BC-46 | IS84YAAY | IS84YR6E | GCI | ILC | 67 | | | stage |
| | | | | | | | | | 2B |
| RT-47 | BC-47 | I35USA9G | I35USR7K | GCI | ILC | 70 | | | stage |
| | | | | | | | | | 2A |
| RT-48 | BC-48 | 17090 | 30738A1 | Asterand | ILC | 50 | | T1cNXM0 | stage 1 |
| RT-49 | BC-49 | 42509 | 42509A1 | Asterand | Ductal | 39 | | T1aN0M0 | stage 1 |
| | | | | | Carcinoma | | | | |
| | | | | | In | | | | |
| | | | | | Situ(DCIS) | | | | |

Benign

| | | | | | | |
|---|---|---|---|---|---|---|
| RT-50 | BE-1 | NNP3QA4V | NNP3QRCW | GCI | FIBROADE | 54 |
| | | | | | NOMA OF | |
| | | | | | THE | |
| | | | | | BREAST | |
| RT-51 | BE-2 | QK8IYALU | QK8IYRW1 | GCI | FIBROADE | 41 |
| | | | | | NOMA OF | |
| | | | | | THE | |
| | | | | | BREAST | |
| RT-52 | BE-3 | ZT15MAMR | ZT15MR2Y | GCI | FIBROADE | 34 |
| | | | | | NOMA OF | |
| | | | | | THE | |
| | | | | | BREAST | |
| RT-53 | BE-4 | 11975 | 15478B1 | Asterand | Fibroadenoma | 24 |

Normal

| | | | | | | |
|---|---|---|---|---|---|---|
| RT-54 | BN-1 | 57-B-N | A609233 | Biochain | Normal PM | 34 |
| RT-55 | BN-2 | 59-B-N | A607155 | Biochain | Normal PM | 35 |
| RT-56 | BN-3 | 60-B-N | A609234 | Biochain | Normal PM | 36 |
| RT-57 | BN-4 | 63-Am-N | 26486 | Ambion | Normal PS | 43 |
| RT-58 | BN-5 | 66-Am-N | 36678 | Ambion | Normal PM | 45 |
| RT-59 | BN-6 | 64-Am-N | 23036 | Ambion | Normal PM | 57 |
| RT-60 | BN-7 | 56-B-N | A609235 | Biochain | Normal PM | 59 |
| RT-61 | BN-8 | 65-Am-N | 31410 | Ambion | Normal PM | 63 |
| RT-62 | BN-9 | 67-Am-N | 073P010602086A | Ambion | Normal PM | 64 |
| RT-63 | BN-10 | 58-B-N | A609232 | Biochain | Normal PM | 65 |
| RT-64 | BN-11 | DHLR1NIQ | DHLR1R8J | GCI | Normal PS | 40 |
| RT-65 | BN-12 | 14398 | 20021D1 | Asterand | Normal PS | 49 |

### Description for Cluster S57296

Cluster S57296 features 6 transcript(s) and 50 segment(s) of interest, the names for which are given in Tables 201 and 202 respectively. The selected protein variants are given in table 203.

TABLE 201
Transcripts of interest
Transcript Name
S57296_1_T75 (SEQ ID NO: 478)
S57296_1_T76 (SEQ ID NO: 479)
S57296_1_T77 (SEQ ID NO: 480)
S57296_1_T78 (SEQ ID NO: 481)
S57296_1_T79 (SEQ ID NO: 482)
S57296_1_T80 (SEQ ID NO: 483)

TABLE 202
Segments of interest

| Segment Name | |
|---|---|
| S57296_1_N11 (SEQ ID NO: 484) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and S57296_1_T80 |
| | (SEQ ID NO: 483) |
| S57296_1_N14 (SEQ ID NO: 485) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and S57296_1_T80 |
| | (SEQ ID NO: 483) |
| S57296_1_N17 (SEQ ID NO: 486) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and S57296_1_T80 |
| | (SEQ ID NO: 483) |
| S57296_1_N20 (SEQ ID NO: 487) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and S57296_1_T80 |
| | (SEQ ID NO: 483) |
| S57296_1_N27 (SEQ ID NO: 488) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and S57296_1_T80 |
| | (SEQ ID NO: 483) |
| S57296_1_N30 (SEQ ID NO: 489) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and S57296_1_T80 |
| | (SEQ ID NO: 483) |
| S57296_1_N32 (SEQ ID NO: 490) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481) and S57296_1_T79 |
| | (SEQ ID NO: 482). Table 30 |
| S57296_1_N45 (SEQ ID NO: 491) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481) and S57296_1_T79 |
| | (SEQ ID NO: 482) |
| S57296_1_N46 also referred as | S57296_1_T77 (SEQ ID NO: 480) and |
| S57296 B2S seg-44 (SEQ ID NO: 552) | S57296_1_T79 (SEQ ID NO: 482) |
| S57296_1_N47 (SEQ ID NO: 493) | S57296_1_T75 (SEQ ID NO: 478), |
| | S57296_1_T76 (SEQ ID NO: 479) and |
| | S57296_1_T78 (SEQ ID NO: 481) |
| S57296_1_N51 (SEQ ID NO: 494) | S57296_1_T75 (SEQ ID NO: 478), |
| | S57296_1_T76 (SEQ ID NO: 479) and |
| | S57296_1_T78 (SEQ ID NO: 481) |
| S57296_1_N59 (SEQ ID NO: 495) | S57296_1_T75 (SEQ ID NO: 478) and |
| | S57296_1_T76 (SEQ ID NO: 479) |
| S57296_1_N61 (SEQ ID NO: 496) | S57296_1_T78 (SEQ ID NO: 481) |
| S57296_1_N70 (SEQ ID NO: 497); | S57296_1_T78 (SEQ ID NO: 481) |
| also referred as S57296WT seg63 | |
| (SEQ ID NO: 497) | |
| S57296_1_N72 (SEQ ID NO: 498) | S57296_1_T78 (SEQ ID NO: 481) |
| S57296_1_N83 (SEQ ID NO: 499) | S57296_1_T78 (SEQ ID NO: 481) |
| S57296_1_N86 (SEQ ID NO: 500) | S57296_1_T78 (SEQ ID NO: 481) |
| S57296_1_N95 (SEQ ID NO: 501) | S57296_1_T78 (SEQ ID NO: 481) |
| S57296_1_N104 (SEQ ID NO: 502) | S57296_1_T78 (SEQ ID NO: 481) |
| S57296_1_N105 (SEQ ID NO: 503) | S57296_1_T78 (SEQ ID NO: 481) |
| S57296_1_N22 (SEQ ID NO: 504) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and S57296_1_T80 |
| | (SEQ ID NO: 483) |
| S57296_1_N25 (SEQ ID NO: 505) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and S57296_1_T80 |
| | (SEQ ID NO: 483) |
| S57296_1_N35 (SEQ ID NO: 506) | S57296_1_T76 (SEQ ID NO: 479), |
| | S57296_1_T78 (SEQ ID NO: 481) and |
| | S57296_1_T79 (SEQ ID NO: 482) |
| S57296_1_N41 (SEQ ID NO: 507) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and S57296_1_T80 |
| | (SEQ ID NO: 483) |
| S57296_1_N43 (SEQ ID NO: 508) | S57296_1_T75 (SEQ ID NO: 478), |
| | S57296_1_T76 (SEQ ID NO: 479), |
| | S57296_1_T77 (SEQ ID NO: 480), |
| | S57296_1_T78 (SEQ ID NO: 481), |
| | S57296_1_T79 (SEQ ID NO: 482) and |
| | S57296_1_T80 (SEQ ID NO: 483) |
| S57296_1_N49 (SEQ ID NO: 509) | S57296_1_T75 (SEQ ID NO: 478), |
| | S57296_1_T76 (SEQ ID NO: 479) and |
| | S57296_1_T78 (SEQ ID NO: 481) |
| S57296_1_N58 (SEQ ID NO: 510) | S57296_1_T75 (SEQ ID NO: 478), |
| | S57296_1_T76 (SEQ ID NO: 479) and |
| | S57296_1_T7 |
| S57296_1_N63 (SEQ ID NO: 511) | S57296_1_T78 (SEQ ID NO: 481) |
| S57296_1_N64 (SEQ ID NO: 512) | S57296_1_T78 (SEQ ID NO: 481) |
| S57296_1_N65 (SEQ ID NO: 513) | S57296_1_T78 (SEQ ID NO: 481) |
| S57296_1_N67 (SEQ ID NO: 514) | S57296_1_T78 (SEQ ID NO: 481) |
| S57296_1_N75 (SEQ ID NO: 515) | S57296_1_T78 (SEQ ID NO: 481) |
| S57296_1_N77 (SEQ ID NO: 516) | S57296_1_T78 (SEQ ID NO: 481) |
| S57296_1_N78 (SEQ ID NO: 517) | S57296_1_T78 (SEQ ID NO: 481) |
| S57296_1_N81 (SEQ ID NO: 518) | S57296_1_T78 (SEQ ID NO: 481) |
| S57296_1_N84 (SEQ ID NO: 519) | S57296_1_T78 (SEQ ID NO: 481) |
| S57296_1_N87 (SEQ ID NO: 520) | S57296_1_T78 (SEQ ID NO: 481) |
| S57296_1_N88 (SEQ ID NO: 521) | S57296_1_T78 (SEQ ID NO: 481) |
| S57296_1_N89 (SEQ ID NO: 522) | S57296_1_T78 (SEQ ID NO: 481) |
| S57296_1_N90 (SEQ ID NO: 523) | S57296_1_T78 (SEQ ID NO: 481) |
| S57296_1_N93 (SEQ ID NO: 524) | S57296_1_T78 (SEQ ID NO: 481) |
| S57296_1_N94 (SEQ ID NO: 525) | S57296_1_T78 (SEQ ID NO: 481) |
| S57296_1_N96 (SEQ ID NO: 526) | S57296_1_T78 (SEQ ID NO: 481) |
| S57296_1_N97 (SEQ ID NO: 527) | S57296_1_T78 (SEQ ID NO: 481) |
| S57296_1_N98 (SEQ ID NO: 528) | S57296_1_T78 (SEQ ID NO: 481) |
| S57296_1_N99 (SEQ ID NO: 529) | S57296_1_T78 (SEQ ID NO: 481) |
| S57296_1_N100 (SEQ ID NO: 530) | S57296_1_T78 (SEQ ID NO: 481) |
| S57296_1_N101 (SEQ ID NO: 531) | S57296_1_T78 (SEQ ID NO: 481) |
| S57296_1_N102 (SEQ ID NO: 532) | S57296_1_T78 (SEQ ID NO: 481) |
| S57296_1_N103 (SEQ ID NO: 533) | S57296_1_T78 (SEQ ID NO: 481) |

TABLE 203
Proteins of interest

| | Corresponding |
|---|---|
| Protein Name | Transcript(s) |
| S57296_1_P59 (SEQ ID NO: 542) | S57296_1_T78 |
| | (SEQ ID NO: 481) |
| S57296_1_P65 (SEQ ID NO: 543) | S57296_1_T77 |
| | (SEQ ID NO: 480) |
| S57296_1_P85 (SEQ ID NO: 544) | S57296_1_T79 |
| | (SEQ ID NO: 482) |
| S57296_1_P97 (SEQ ID NO: 545) | S57296_1_T80 |
| | (SEQ ID NO: 483) |
| S57296_1_P125 (SEQ ID NO: 546) | S57296_1_T75 |
| | (SEQ ID NO: 478) |
| S57296_1_P127 (SEQ ID NO: 547) | S57296_1_T76 |
| | (SEQ ID NO: 479) |

These sequences are variants of the known protein Herstatin (SEQ ID NO:534) (SwissProt accession identifier Q9UK79_HUMAN (SEQ ID NO:534)), referred to herein as the previously known protein.

The following GO Annotation(s) apply to the previously known protein. The following annotation(s) were found: transmembrane receptor protein tyrosine kinase signaling pathway, which are annotation(s) related to Biological Process; non-membrane spanning protein tyrosine kinase activity, which are annotation(s) related to Molecular Function; and extracellular region, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of S57296) may optionally have one or more of the following utilities, as described with regard to Table 204 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 204
Utilities for Variants of S57296

| Utility | Reason | Reference |
|---|---|---|
| Gene and protein | Detection of | Science. 1989 May 12; 244(4905): 707-12. |
| over expression in | protein (i.e. | Br J Cancer. 1991 Jul; 64(1): 79-83. |
| breast, ovarian, | western, ICH, | J Pathol. 1989 Oct; 159(2): 107-11. |
| stomach, bladder, | etc.) or mRNA | Cancer Lett. 1993 Nov 30; 75(1): 41-4. |
| salivary, lung, | from the tumor | Cancer Res. 1993 May 15; 53(10 Suppl): 2199-203. |
| prostate cancers | (i.e. RT-PCR, | Genes Chromosomes Cancer. 1991 Mar; 3(2): 128-35. |
| and Paget disease | Northern blot, | Gynecol Oncol. 2004 Nov; 95(2): 336-40. |
| of the vulva and | etc.). | Nature 393: 83-85, 1998 |
| the female breast | | |
| Indication for | Tumors that | Cancer Invest. 2004; 22(6): 858-65. |
| ErbB2 directed | overexpress the | Int J Radiat Oncol Biol Phys. 2005 Jan 1; 61(1): 203-11. |
| therapy | protein are | Cancer Invest. 2004; 22(5): 706-12. |
| (antibodies, small | targets for | Breast Cancer. 2004; 11(2): 105-15. |
| molecules, etc.) | therapy. | Semin Oncol. 2004 Oct; 31(5 Suppl 10): 29-34. |
| Gene over | Gene, mRNA | Slamon DJ, Clark GM, Wong SG et al. Human breast cancer: |
| expression can | over expression | correlation of relapse and survival with amplification of the |
| predict breast | detection. | Her-2/neu oncogene. Science 1987; 235: 177-182. |
| cancer prognosis | Protein levels in | Berger MS, Locher GW, Saurer S et al. Correlation of c-erb B2 |
| and response to | tumor or in | gene amplification and protein expression in human breast |
| anti cancer | serum. Detection | carcinoma with nodal status and nuclear grading. Cancer |
| treatment. | of protein is | Res 1988; 48: 1238-1243. |
| | indicative of | van de Vivjer MJ, Peterse JL, Mooi WJ et al. Neu-protein |
| | poor prognosis | overexpression in breast cancer. N Engl J Med |
| | relative to non | 1988; 319: 1239-1245. |
| | expressing | Heintz NH, Leslie KO, Rogers LA et al. Amplification of the c- |
| | tumors. | erb B-2 oncogene in prognosis of breast adenocarcinoma. |
| | | Arch Pathol Lab Med 1990; 114: 160-163. |
| | | Tsuda H, Hirohashi S, Shimosato Y et al. Correlation between |
| | | histologic grade of malignancy and copy number of c-erbB- |
| | | 2 gene in breast carcinoma. A retrospective analysis of 176 |
| | | cases. Cancer 1990; 65: 1794-1800. |
| | | Borg A, Tandon AK, Sigurdsson H et al. HER-2/neu |
| | | amplification predicts poor survival in node-positive breast |
| | | cancer. Cancer Res 1990; 50: 4332-4337. |
| | | Paik S, Hazan R, Fisher ER et al. Pathologic findings from the |
| | | nations′ surgical adjuvant breast and bowel project: |
| | | prognostic significance of erb B2 protein overexpression in |
| | | primary breast cancer. J Clin Oncol 1990; 8: 103-112. |
| | | Battifora H, Gaffey M, Esteban J et al. Immunohistochemical |
| | | assay of neu/c-erb B-2 oncogene product in paraffin- |
| | | embedded tissues in early breast cancer: Retrospective |
| | | follow-up study of 245 stage I and II cases. Modern Pathol |
| | | 1991; 4: 466-474. |
| | | Kallioniemi OP, Holli K, Visakorpi T et al. Association of C-erb |
| | | B2 protein over-expression with high rate of cell |
| | | proliferation, increased risk of visceral metastasis and poor |
| | | long-term survival in breast cancer. Int J Cancer |
| | | 1991; 49: 650-655. |
| | | Clark GM, McGuire WL. Follow-up study of HER-2/neu |
| | | amplification in primary breast cancer. Cancer Res |
| | | 1991; 51: 944-948. |
| | | Lovekin C, Ellis IO, Locker A et al. C-erb B2 oncoprotein |
| | | expression in primary and advanced breast cancer. Br J |
| | | Cancer 1991; 63: 439-443. |
| | | McCann AH, DeDervan TA, O′Regan M et al. Prognostic |
| | | significance of C-erb B2 and estrogen receptor status in |
| | | human breast cancer. Cancer Res 1991; 51: 3296-3303. |
| | | Dykens R, Corbett IP, Henry J et al. Long term survival in |
| | | breast cancer related to overexpression of the C-erb B2 |
| | | oncoprotein: an immunohistochemical study using |
| | | monoclonal antibody NCL-CB11. J Pathol 1991; 163: 105-110. |
| | | Rilke F, Colnaghi MI, Cascinelli N et al. Prognostic significance |
| | | of HER-2/neu expression in breast cancer and its |
| | | relationship to other prognostic factors. Int J Cancer |
| | | 1991; 49: 44-49. |
| | | Winstanley J, Cooke T, Murray GD et al. The long term |
| | | prognostic significance of C-erb B2 in primary breast |
| | | cancer. Br J Cancer 1991; 63: 447-450. |
| | | O′Reilly SM, Barnes DM, Camplejohn RS et al. The |
| | | relationship between C-erb B2 expression, and s-phase |
| | | fraction in prognosis in breast cancer. Br J Cancer |
| | | 1991; 63: 444-446. |
| | | Paterson MC, Dietrich KD, Danyluk J et al. Correlation between |
| | | C-erb B2 amplification and risk of recurrent disease in |
| | | node-negative breast cancer. Cancer Res 1991; 51: 556-567. |
| | | Toikkanen S, Helin H, Isola J et al. Prognostic significance of |
| | | Her-2 oncoprotein expression in breast cancer: a 30-year |
| | | follow up. J Clin Oncol 1992; 10: 1044-1048. |
| | | Molina R, Ciocca DR, Candon AK et al. Expression of HER- |
| | | 2/neu oncoprotein in breast cancer: a comparison of |
| | | immunohistochemical and western blot techniques. |
| | | Anticancer Res 1992; 12: 1965-1991. |
| | | Noguchi M, Koyasaki M, Ohta N et al. c-erb B-2 oncoprotein |
| | | expression versus internal mammary lymph node |
| | | metastases as additional prognostic factors in patients with |
| | | axillary lymph node-positive breast cancer. Cancer |
| | | 1992; 69: 2953-2960. |
| | | Allred DC, Clark GM, Tandon AK et al. HER-2/neu node- |
| | | negative breast cancer: prognostic significance of |
| | | overexpression influenced by the presence of in-situ |
| | | carcinoma. J Clin Oncol 1992; 10: 599-605. |
| | | Babiak J, Hugh J, Poppeme S. Significance of c-erb B-2 |
| | | amplification in DNA aneuploidy. Analysis in 78 patients |
| | | with node-negative breast cancer. Cancer 1992; 70: 770-776. |
| | | Tiwari RK, Borgen PI, Wong GY et al. HER-2/neu |
| | | amplification and overexpression in primary human breast |
| | | cancer is associated with early metastasis. Anticancer Res |
| | | 1992; 12: 419-426. |
| | | Gusterson BA, Gelber RD. Goldhirsch A et al. Prognostic |
| | | importance of C-erb B2 expression in breast cancer. J Clin |
| | | Oncol 1992; 10: 1049-1056. |
| | | Bianchi S, Paglierani M, Zampi G et al. Prognostic significance |
| | | of C-erb B2 expression in node negative breast cancer. Br J |
| | | Cancer 1993; 67: 625-629. |
| | | Press MF, Pike MC, Chazin VR et al. Her-2/neu expression in |
| | | node-negative breast cancer: direct tissue quantification by |
| | | computerized image analysis and association of |
| | | overexpression with increased risk of recurrent disease. |
| | | Cancer Res 1993; 53: 4960-4970. |
| | | Seshadri R, Firgaira FA, Horsfall DJ et al. Clinical significance |
| | | of Her-2/neu oncogene amplification in primary breast |
| | | cancer. J Clin Oncol 1993; 11: 1936-1942. |
| | | Descotes F, Pavy J-J, Adessi GL. Human breast cancer: |
| | | correlation study between Her-2/neu amplification and |
| | | prognostic factors in an unselected population. Anticancer |
| | | Res 1993; 13: 119-124. |
| | | Giai M, Roagna R, Ponzone R et al. Prognostic and predictive |
| | | relevance of C-erb B2 and ras expression in node-positive |
| | | and negative breast cancer. Anticancer Res 1994; 14: 1441-1450. |
| | | Muss HB, Thor AD, Berry DA et al. Cerb-B2 expression and |
| | | response to adjuvant therapy in women with node-positive |
| | | early breast cancer. N Engl J Med 1994; 330: 1260-1266. |
| | | Tetu B, Brisson J. Prognostic significance of Her-2/neu |
| | | oncogene expression in node-positive breast cancer. The |
| | | influence of the pattern of immunostaining and adjuvant |
| | | therapy. Cancer 1994; 73: 2359-2365. |
| | | Hartmann LC, Ingle JN, Wold LE et al. Prognostic value of |
| | | CerbB2 overexpression in axillary lymph node-positive |
| | | breast cancer. Results from a randomized adjuvant |
| | | treatment protocol. Cancer 1994; 74: 2956-2963. |
| | | Jacquemier J, Penault-Llorca P, Viens P et al. Breast cancer |
| | | response to adjuvant chemotherapy in correlation with erb |
| | | B2 and p53 expression. Anticancer Res 1994; 14: 2773-2778. |
| | | Marks JR, Humphrey PA, Wu K at al. Overexpression of p53 |
| | | and Her-2/neu proteins as prognostic markers in early stage |
| | | breast cancer. Ann Surg 1994; 219: 332-341. |
| | | Rosen PP, Lesser ML, Arroyo CD et al. Immunohistochemical |
| | | detection of Her-2/neu expression in patients with axillary |
| | | lymph node-negative breast carcinoma. A study of |
| | | epidemiologic risk factors, histologic features and |
| | | prognosis. Cancer 1995; 75: 1320-1326. |
| | | Quenel N, Wafflart J, Bonichon F et al. The prognostic value of |
| | | c-erbB2 in primary breast carcinomas: a study on 942 cases. |
| | | Breast Cancer Res Treat 1995; 35: 283-291. |
| | | Sundblad AS, Pellicer EM, Ricci L. Carcinoembryonic |
| | | expression in stages I and II breast cancer; its relationship |
| | | with clinicopathologic factors. Hum Pathol 1996; 27: 297-300. |
| | | O′Malley FP, Saad Z, Kerkvliet N et al. The predictive power of |
| | | semiquantitative immunohistochemical assessment of p53 |
| | | and C-erbB2 in lymph node-negative breast cancer. Hum |
| | | Pathol 1996; 27:955-963. |
| | | Hieken TJ, Mehta RR, Shilkaitis A et al. Her-2/neu and p53 |
| | | expression in breast cancer: valid prognostic markers when |
| | | assessed by direct immunoassay, but not by |
| | | immunochemistry. Proc Annu Meet Am Soc Clin Oncol |
| | | 1996; 15: 113a. |
| Combinations of | Combination of | Stem Cells. 1998; 16(6): 413-28. |
| prognostic factors | detection of | Zafrani B, Leroyer A, Fourquet A et al. Mammographically- |
| in breast cancer | erbb2 with | detected ductal in-situ carcinoma of the breast analyzed with a |
| | estrogen and | new classification. Sem Diagn Pathol 1994; 11: 208-214. |
| | progesterone | Brower FT, Abmed 5, Tartter P et al. Prognostic variables in |
| | receptors, cell | invasive breast cancer: Contribution of comedo versus non- |
| | proliferation | comedo in-situ component. Ann Surg Oncol 1995; 2: 440-444. |
| | index, DNA | Leal CB, Schmitt FC, Bento NJ et al. Ductal carcinoma in-situ of |
| | ploidity, myc, | the breast. Histologic categorization and its relationship to |
| | ras, (and other | ploidy and immunohistochemical expression of hormone |
| | oncogens), p53 | receptors, p53 and C erb B-2 protein. Cancer 1995; 75: 2123-2131. |
| | (and other tumor | Bobrow WLG, Happerfield LC, Gregory WM et al. Ductal |
| | suppressors), | carcinoma in-situ: assessment of necrosis in nuclear morphology |
| | cathepsin D, | and their association with biological markers. J Pathol |
| | PAI1 or uPAI. | 1995; 176: 333-341. |
| Detection of | Protein over | DePotter CR, Schelhout AM, Verbeeck P et al. neu |
| Ductal Carcinoma | expression in | overexpression correlates with extent of disease in large cell |
| in Situ - comedo- | tumors and | ductal in situ carcinoma of the breast. Hum Pathol 1995; 26: 601-606. |
| carcinoma variant | biopsies. | Inaji H, Koyama H, Motomura K et al. Differential distribution |
| | | of erb B2 and pS2 proteins in ductal carcinoma in-situ of the |
| | | breast. Breast Cancer Res Treat 1996; 37: 89-92. |
| | | Albonico G, Querzoli P, Ferretti S et al. Biophenotypes of breast |
| | | carcinoma in-situ defined by image analysis of biological |
| | | parameters. Pathol Res Pract 1996; 192: 117-123. |
| | | Bose S, Lesser ML, Norton L et al. Immunophenotype of |
| | | intraductal carcinoma. Arch Pathol Lab Med 1996; 120: 81-85. |
| | | Moreno A, Lloveras B, Figueras A et al. Ductal carcinoma in- |
| | | situ of the breast: Correlation between histologic classification |
| | | and biologic markers. Mod Pathol 1997; 10: 1088-1092. |
| | | Mack L, Kerkzelit N, Doig G et al. Relationship of a new |
| | | histological categorization of ductal carcinoma in-situ of the |
| | | breast with size and the immunohistochemical expression of |
| | | p53, C-erb B2, bcl2 and ki-67. Hum Pathol 1997; 28: 974-979. |
| Serum levels of | higher levels of | Shepard HM, Lewis GD, Sarup JC et al. Monoclonal antibody |
| ErbB2 can be used | erbb2 in serum | therapy of human cancer: taking the Her-2 proto-oncogene to |
| for detection, | taken from breast | the clinic. J Clin Immunol 1991; 11: 117-127. |
| prognosis, | cancer patients | Zhang L, Chang C, Bacus SS et al. Suppressed transformation |
| treatment | indicate poor | and induced differentiation of Her-2/neu-overexpressing breast |
| selection and | prognosis and | cancer cells by emodin. Cancer Res 1995; 55: 3890-3896. |
| treatment response | treatment | Valone FH, Kaufman PA, Guyre PM et al. Phase Ia/Ib trial of |
| markers. | resistance. | bispecific antibody MDX-210 in patients with advanced breast |
| | | or ovarian cancer that overexpresses the proto-oncogene HER- |
| | | 2/neu. J Clin Oncol 1995; 13: 2281-2292. |
| | | Jinno H, Ueda M, Enomoto K et al. Effectiveness of an |
| | | Adriamycin immunoconjugate that recognizes the CerbB2 |
| | | product on breast cancer cell lines. Surg Today 1996; 26: 501-507. |
| | | Baselga J, Tripathy D, Mendelsohn J et al. Phase II study of |
| | | weekly intravenous recombinant humanized anti p185 Her-2 |
| | | monoclonal antibody in patients with Her-2/neu overexpressing |
| | | metastatic breast cancer. J Clin Oncol 1996; 14: 737-744. |
| | | Liu X, Pogo BG. Inhibition of erbB2 positive breast cancer cell |
| | | growth by erbB2 antisense oligonucleotides. Antisense Nucleic |
| | | Acid Drug Devel 1996; 6: 9-16. |
| | | Wright M, Grim J, Deshane J et al. An intracellular anti-erbB-2 |
| | | single-chain antibody is specifically cytotoxic to human breast |
| | | carcinoma cells overexpressing erbB-2. Gene Ther 1997; 4: 317-322. |
| | | Heijnen IA, Buhring HJ, Deo YN et al. The clinical studies with |
| | | Fc (Gamma) R by specific antibodies and granulocyte colony- |
| | | stimulating factor-primed neutrophils as effector cells against |
| | | HER-2/neu overexpression in breast cancer. Cancer Res |
| | | 1997; 57: 696-701. |
| | | Witters LM, Kumar R, Chinchilli ZM et al. Enhanced anti- |
| | | proliferative activity of the combination of tamoxifen plus HER- |
| | | 2-neu antibody. Breast Cancer Res Treat 1997; 42: 1-5. |
| | | Cobleigh MA, Vogel CL, Tripathy NJ et al. Efficacy and safety |
| | | of Herceptin ® (humanized anti-human HER-2 antibody) as a |
| | | single agent in 222 women with HER2 overexpression who |
| | | relapsed following chemotherapy for metastatic breast cancer. |
| | | Proc Am Soc Clin Oncol 1998; 17: 97a. |
| | | Slamon D, Leyland-Jones B, Shak S et al. Addition of |
| | | Herceptin ® (humanized anti HER2 antibody) to first line |
| | | chemotherapy for HER2 overexpressing metastatic breast |
| | | cancer markedly increases anticancer activity: a randomized, |
| | | multinational controlled phase III trial. Proc Am Soc Clin Oncol |
| | | 1998; 17: 98a. |
| Diagnosis of | Protein over | Am J Obstet Gynecol. 2004 Dec; 191(6): 2106-13. |
| placenta accreta | expressed in | |
| | syncytio- | |
| | trophoblast of | |
| | placenta accrete | |
| | cases. | |
| Graves' disease | MRNA over | Biochim Biophys Acta. 2004 Aug 4; 1673(3): 194-200. |
| detection | expressed in | |
| | thyroid tissue | |
| | from patients | |
| | with Graves's | |
| | disease. | |
| Risk assessment | Preeclampsia is | Clin Chem Lab Med. 2004 Feb; 42(2): 142-6. |
| for preeclampsia | associated with | |
| pregnancies. | increased serum | |
| | levels of | |
| | ERRB2(p105) | |
| | concentration. | |
| Detection of | Oligodendrocytes | J Neuroimmunol. 1999 Dec; 100(1-2): 233-42. |
| multiple sclerosis | of MS patients | Brain Pathol. 1999 Apr; 9(2): 293-311. |
| | expresses higher | |
| | levels of ERBB2 | |
| Detection of lung | A higher | Clin Cancer Res. 2003 Jul; 9(7): 2605-12 |
| cancer is pleural | incidence of | |
| effusion | HER-2/neu | |
| | expression | |
| | beyond the | |
| | cutoff point (5.5 ng/ml) | |
| | in pleural | |
| | effusions was | |
| | also found in | |
| | patients whose | |
| | IHC H-scores | |
| | were >50 | |

According to other optional embodiments of the present invention, variants of this cluster according to the present invention (amino acid and/or nucleic acid sequences of S57296) may optionally have one or more of the following utilities, some of which are related to utilities described above. It should be noted that these utilities are optionally and preferably suitable for human and non-human animals as subjects, except where otherwise noted.

A non-limiting example of such a utility is the detection, diagnosis and/or determination of rheumatoid arthritis. The method comprises detecting a S57296 variant, for example a variant protein, protein fragment, peptide, polynucleotide, polynucleotide fragment and/or oligonucleotide as described herein, optionally and preferably in a serum sample and/or in synoviocytes. Such use of the known protein is described with regard to European Patent Application No. EP1275398, hereby incorporated by reference as if fully set forth herein.

Another non-limiting example of such a utility is the detection, diagnosis and/or determination of breast cancer. The method comprises detecting a S57296 variant, for example a variant protein, protein fragment, peptide, polynucleotide, polynucleotide fragment and/or oligonucleotide as described herein, optionally and preferably in a serum sample and/or in a saliva sample. Such use of the known protein is described with regard to U.S. Pat. No. 6,294,349, hereby incorporated by reference as if fully set forth herein.

Cluster S57296 belongs to a family of proteins including but not limited to, EGFR, ErbB3 and ErbB4. These functions are described below; one or more variants of cluster S57296 may optionally have one or more diagnostic utilities related to these functions. ErbB3 is overexpressed in a subset of human mammary tumors. For example, high expression is associated with invasion in 41 cases of oral squamous cell carcinoma. —Shintani S. Funayama T. Yoshihama Y. Alcalde R E. Matsumura T Cancer Lett 95: 79-83 (1995). High expression is associated with lymph node metastasis—Shintani S. Funayama T. Yoshihama Y. Alcalde R E. Matsumura T Cancer Lett 95: 79-83 (1995); Overexpressing cells sensitive to H/PE40H/PE40 is a heregulin beta 2 PE40 ligand toxin. —Fiddes R J. Janes P W. Sanderson G M. Sivertsen S P. Sutherland R L. Daly R J. Cell Growth Differ 6: 1567-1577 (1995); Gene copy #<1.75=> decreased survival In primary breast cancer patients. —Brandt B. Vogt U. Schlotter C M. Jackisch C. Werkmeister R. Thomas M. von Eiff M. Bosse U. Assmann G. Zanker K S Gene 159: 35-42 (1995).

C17orf37 (NM_{—}032339; NP_{—}115715)(also called XTP4, MGC14832) is antisense, tail to tail, to ErbB2, and was found to be transactivated by hepatitis B virus X antigen (HBxAg). Therefore, its expression might serve as indication for hepatitis B infection. (NCBI sequence reference—gi:29468235). This gene may therefore be co-regulated with one or more S57296 variants according to the present invention, and hence may have one or more utilities of S57296 variants according to the present invention as described herein.

PERLD1 (NM_{—}033419; NP_{—}219487) is antisense, head to head (probably co-regulated), to ErbB2, and is predicted to code for seven-transmembrane receptor with the N-terminal six-cysteine domain and an N-glycosylation site. Also MYC, ERBB2, MET, FGFR2, CCNE1, MYCN, WNT2, CD44, MDM2, NCOA3, IQGAP1 and STK6 loci are amplified in human gastric cancer. It has been reported that PERLD1 is co-amplified with ERBB2 gene in human gastric cancer. (Katoh M, Katoh M Int J Oncol. 2003 June; 22(6):1369-74). This gene may therefore be co-regulated with one or more S57296 variants according to the present invention, and hence may have one or more utilities of S57296 variants according to the present invention as described herein.

The PPP1R1B-STARD3-TCAP-PNMT-PERLD1-ERBB2-MGC14832-GRB7 locus at human chromosome 17q12 is frequently amplified in human gastric cancer and breast cancer. (Katoh M, Katoh M Int J Oncol. 2004 April; 24(4):757-63). This gene may therefore be co-regulated with one or more S57296 variants according to the present invention, and hence may have one or more utilities of S57296 variants according to the present invention as described herein.

Table 205 below describes diagnostic utilities for the cluster S57296 that were found through microarrays, including the statistical significance thereof and a reference. One or more S57296 variants according to the present invention may optionally have one or more of these utilities.

TABLE 205

| | Statistical | |
|---|---|---|
| Diagnostic utility | significance | reference |
| Gene over expression in breast ductal | 0.034 | Perou CM, Botstein D Nature |
| carcinoma (vs. normal breast or fibroadenoma). | | (2000) Molecular portraits of |
| | | human breast tumours. |
| Gene over expression in breast adeno- | 0.01 to 0.38 | Sorlie T, Borresen-Dale AL |
| carcinoma and carcinoma (vs normal breast and | | PNAS (2001) Gene expression |
| other cancers). | | patterns of breast carcinomas |
| | | distinguish tumor subclasses |
| | | with clinical implications. |
| | | Ramaswamy S; Golub TR |
| | | PNAS (2001) Multiclass cancer |
| | | diagnosis using tumor gene |
| | | expression signatures. |
| Gene over expressed in sporadic breast cancers | 4.6e⁻⁵ | Van't Veer LJ, Friend SH |
| (vs. BRCA1 & 2 positive cancers). | | Nature (2002) Gene expression |
| | | profiling predicts clinical |
| | | outcome of breast cancer. |
| Gene over expressed in lung adeno-carcinoma | 1e⁻⁴ to 9.2e⁻¹⁵ | Beer DJ, Hanash SM Nature |
| (vs. normal lung, and other lung cancers). | | Medicine (2002) Gene- |
| | | expression profiles predict |
| | | survival of patients with lung |
| | | adenocarcinoma. |
| | | Garber ME, Petersen I PNAS |
| | | (2001) Diversity of gene |
| | | expression in adenocarcinoma |
| | | of the lung. |
| | | Bhattacharjee A, Meyerson M |
| | | PNAS (2001) Classification of |
| | | human lung carcinomas by |
| | | mRNA expression profiling |
| | | reveals distinct adenocarcinoma |
| | | subclasses. |
| Gene over expression in Ovarian | 0.001 to 1e⁻⁵ | Ramaswamy S; Golub TR |
| adenocarcinoma (vs. normal ovary and other | | PNAS (2001) Multiclass cancer |
| cancer types) | | diagnosis using tumor gene |
| | | expression signatures. |
| | | Welsh JB, Hampton GM PNAS |
| | | (2001) Analysis of gene |
| | | expression profiles in normal |
| | | and neoplastic ovarian tissue |
| | | samples identifies candidate |
| | | molecular markers of epithelial |
| | | ovarian cancer. |
| Gene over expressed in Pancreatic | 0.019 to 0.006 | Am J Pathol (2003) Exploration |
| Adenocarcinoma (vs. normal pancreas). | | of global gene expression |
| | | patterns in pancreatic |
| | | adenocarcinoma using cDNA |
| | | microarrays. |
| | | Ramaswamy S; Golub TR |
| | | PNAS (2001) Multiclass cancer |
| | | diagnosis using tumor gene |
| | | expression signatures. |

Also, microarrays have shown that one or more S57296 variants according to the present invention may be overexpressed in lung cancer, preferably adenocarcinoma.

Other non-limiting exemplary utilities for S57296 variants according to the present invention are described in greater detail below and also with regard to the previous section on clinical utility.

Also, it should be noted that one or more S57296 variants optionally and preferably may have one or more therapeutic utilities, as described with regard to PCT Application No. WO 05/033133, hereby incorporated by reference as if fully set forth herein.

Cluster S57296 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

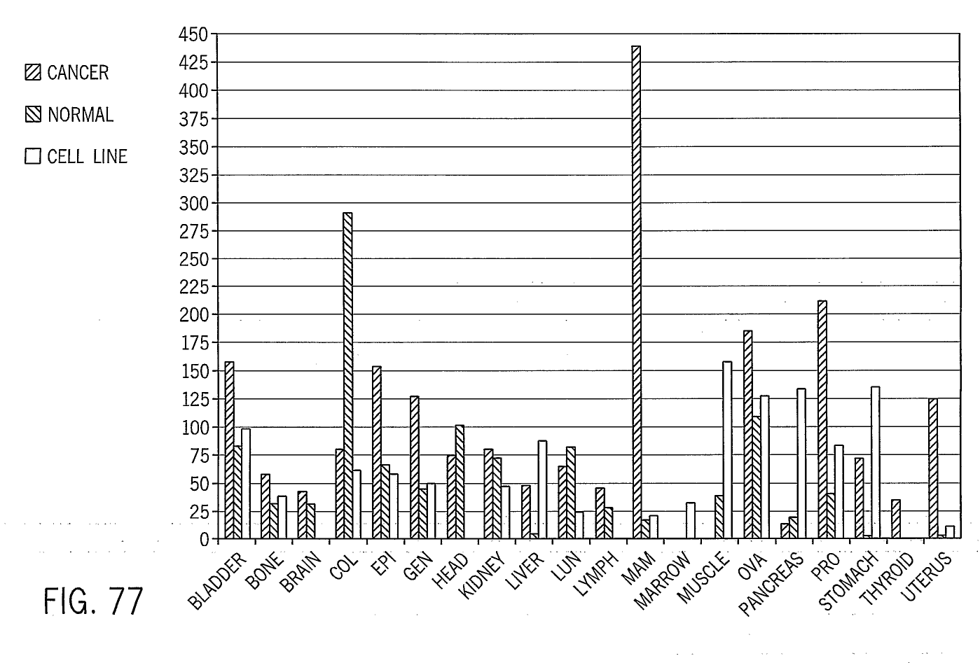

Overall, the following results were obtained as shown with regard to the histograms in FIG. 77 and Table 206. This cluster is overexpressed (at least at a minimum level) in the following pathological conditions: a mixture of malignant tumors from different tissues, uterine malignancies, breast malignant tumors and epithelial malignant tumors. P values and ratios for expression in cancerous tissue are described in table 207

TABLE 206
Normal tissue distribution

| Name of Tissue | Number |
|---|---|
| Brain | 31 |
| Ovary | 109 |
| Bladder | 82 |
| Lung | 82 |
| Pancreas | 20 |
| Liver | 4 |
| Prostate | 41 |
| General | 44 |
| bone marrow | 0 |
| Thyroid | 0 |
| Muscle | 39 |
| Uterus | 4 |
| Colon | 291 |
| Kidney | 72 |
| lymph nodes | 28 |
| Breast | 17 |
| head and neck | 101 |
| Stomach | 3 |
| Epithelial | 66 |
| Bone | 31 |

TABLE 207
P values and ratios for expression in cancerous tissue

| Name of Tissue | P1 | P2 | SP1 | R3 | SP2 | R4 |
|---|---|---|---|---|---|---|
| Brain | 3.3e−01 | 6.0e−01 | 3.9e−01 | 1.3 | 8.6e−01 | 0.6 |
| Ovary | 2.0e−01 | 1.3e−01 | 3.1e−01 | 1.4 | 3.7e−01 | 1.3 |
| Bladder | 3.6e−01 | 3.6e−01 | 3.5e−01 | 1.6 | 4.1e−01 | 1.4 |
| Lung | 7.9e−01 | 8.6e−01 | 7.9e−01 | 0.8 | 9.6e−01 | 0.6 |
| Pancreas | 6.2e−01 | 2.8e−01 | 8.1e−01 | 0.8 | 1.8e−01 | 1.8 |
| liver | 3.3e−01 | 4.9e−01 | 1 | 1.8 | 3.4e−01 | 2.3 |
| prostate | 6.4e−01 | 6.1e−01 | 7.3e−03 | 2.5 | 2.0e−02 | 2.2 |
| general | 2.8e−05 | 1.9e−04 | 8.4e−15 | 2.6 | 3.7e−08 | 1.8 |
| bone marrow | 1 | 7.1e−01 | 1 | 1.0 | 5.4e−01 | 1.9 |
| Thyroid | 2.4e−01 | 2.4e−01 | 1 | 1.3 | 1 | 1.3 |
| muscle | 8.5e−01 | 6.1e−01 | 1 | 0.3 | 1.7e−01 | 1.3 |
| uterus | 8.9e−03 | 4.4e−02 | 1.1e−02 | 5.1 | 6.8e−02 | 3.2 |
| colon | 7.5e−01 | 7.0e−01 | 1 | 0.3 | 1 | 0.3 |
| kidney | 3.5e−01 | 3.0e−01 | 5.4e−01 | 1.1 | 6.4e−01 | 0.9 |
| lymph nodes | 7.4e−01 | 8.7e−01 | 4.9e−01 | 1.4 | 8.2e−01 | 0.7 |
| breast | 1.1e−01 | 1.2e−01 | 2.5e−04 | 3.7 | 1.1e−02 | 2.3 |
| head and neck | 3.4e−01 | 5.0e−01 | 1 | 0.9 | 1 | 0.6 |
| stomach | 1.2e−01 | 8.6e−02 | 2.5e−01 | 2.7 | 5.2e−02 | 3.2 |
| epithelial | 8.9e−03 | 2.2e−02 | 3.9e−07 | 2.0 | 2.3e−03 | 1.5 |
| bone | 2.3e−01 | 4.4e−01 | 6.4e−01 | 1.4 | 6.5e−01 | 1.2 |

As noted above, cluster S57296 features 6 transcript(s), which were listed in Table 201 above. These transcript(s) encode for protein(s) which are variant(s) of protein Herstatin (SEQ ID NO:534). A description of each variant protein according to the present invention is now provided.

Variant protein S57296_{—}1_P59 (SEQ ID NO:542) according to the present invention has an amino acid sequence; it is encoded by transcript(s) S57296_{—}1_T78 (SEQ ID NO:481). An alignment is given to the known protein (Herstatin (SEQ ID NO:534)). One or more alignments to one or more previously published protein sequences are given in the alignment table located on the attached CDROM.

### 1. Comparison Report Between S57296_{—}1_P59 (SEQ ID NO:542) and ERB2_HUMAN (SEQ ID NO:538):

A. An isolated chimeric polypeptide encoding for S57296_{—}1_P59 (SEQ ID NO:542), comprising a first amino acid sequence being at least 90% homologous to MELAALCRWGLLLALLPPGAASTQVCTGTDMKLRLPASPETHLDMLRHLYQGCQVVQGNLELTY LPTNASLSFLQDIQEVQGYVLIAHNQVRQVPLQRLRIVRGTQLFEDNYALAVLDNGDPLNNTTPVT GASPGGLRELQLRSLTEILKGGVLIQRNPQLCYQDTILWKDIFHKNNQLALTLIDTNRSRACHPCSP MCKGSRCWGESSEDCQSLTRTVCAGGCARCKGPLPTDCCHEQCAAGCTGPKHSDCLACLHFNHS GICELHCPALVTYNTDTFESMPNPEGRYTFGASCVTACPYNYLSTDVGSCTLVCPLHNQEVTAEDG TQRCEKCSKPCARVCYGLGMEHLREVRAVTSANIQEFAGCKKIFGSLAFLPESFDG corresponding to amino acids 1-383 of ERB2_HUMAN (SEQ ID NO:538), which also corresponds to amino acids 1-383 of S57296_{—}1_P59 (SEQ ID NO:542), a second amino acid sequence being at least 70%, optionally at least 80%, preferably at least 85%, more preferably at least 90% and most preferably at least 95% homologous to a polypeptide having the sequence VSLCQQAGVQWYDLGSLQPLPPGFKQFSCLSLLSSWDYR (SEQ ID NO: 672) corresponding to amino acids 384-422 of S57296_{—}1_P59 (SEQ ID NO:542), and a third amino acid sequence being at least 90% homologous to DPASNTAPLQPEQLQVFETLEEITGYLYISAWPDSLPDLSVFQNLQVIRGRILHNGAYSLTLQGLGIS WLGLRSLRELGSGLALIHHNTHLCFVHTVPWDQLFRNPHQALLHTANRPEDECVGEGLACHQLC ARGHCWGPGPTQCVNCSQFLRGQECVEECRVLQGLPREYVNARHCLPCHPECQPQNGSVTCFGPE ADQCVACAHYKDPPFCVARCPSGVKPDLSYMPIWKFPDEEGACQPCPINCTHSCVDLDDKGCPAE QRASPLTSIISAVVGILLVVVLGVVFGILIKRRQQKIRKYTMRRLLQETELVEPLTPSGAMPNQAQM RILKETELRKVKVLGSGAFGTVYKGIWIPDGENVKIPVAIKVLRENTSPKANKEILDEAYVMAGVG SPYVSRLLGICLTSTVQLVTQLMPYGCLLDHVRENRGRLGSQDLLNWCMQIAKGMSYLEDVRLV HRDLAARNVLVKSPNHVKITDFGLARLLDIDETEYHADGGKVPIKWMALESILRRRFTHQSDVWS YGVTVWELMTFGAKPYDGIPAREIPDLLEKGERLPQPPICTIDVYMIMVKCWMIDSECRPRFRELV SEFSRMARDPQRFVVIQNEDLGPASPLDSTFYRSLLEDDDMGDLVDAEEYLVPQQGFFCPDPAPGA GGMVHHRHRSSSTRSGGGDLTLGLEPSEEEAPRSPLAPSEGAGSDVFDGDLGMGAAKGLQSLPTH DPSPLQRYSEDPTVPLPSETDGYVAPLTCSPQPEYVNQPDVRPQPPSPREGPLPAARPAGATLERPK TLSPGKNGVVKDVFAFGGAVENPEYLTPQGGAAPQPHPPPAFSPAFDNLYYWDQDPPERGAPPST FKGTPTAENPEYLGLDVPV corresponding to amino acids 384-1255 of ERB2_HUMAN (SEQ ID NO:538), which also corresponds to amino acids 423-1294 of S57296_{—}1_P59 (SEQ ID NO:542), wherein said first amino acid sequence, second amino acid sequence and third amino acid sequence are contiguous and in a sequential order.

B. An isolated polypeptide encoding for an edge portion of S57296_{—}1_P59 (SEQ ID NO:542), comprising an amino acid sequence being at least 70%, optionally at least about 80%, preferably at least about 85%, more preferably at least about 90% and most preferably at least about 95% homologous to the sequence VSLCQQAGVQWYDLGSLQPLPPGFKQFSCLSLLSSWDYR (SEQ ID NO: 672) of S57296_{—}1_P59 (SEQ ID NO:542).

### 2. Comparison Report Between S57296_{—}1_P59 (SEQ ID NO:542) and NP_{—}004439 (SEQ ID NO:540):

A. An isolated chimeric polypeptide encoding for S57296_{—}1_P59 (SEQ ID NO:542), comprising a first amino acid sequence being at least 90% homologous to MELAALCRWGLLLALLPPGAASTQVCTGTDMKLRLPASPETHLDMLRHLYQGCQVVQGNLELTY LPTNASLSFLQDIQEVQGYVLIAHNQVRQVPLQRLRIVRGTQLFEDNYALAVLDNGDPLNNTTPVT GASPGGLRELQLRSLTEILKGGVLIQRNPQLCYQDTILWKDIFHKNNQLALTLIDTNRSRACHPCSP MCKGSRCWGESSEDCQSLTRTVCAGGCARCKGPLPTDCCHEQCAAGCTGPKHSDCLACLHFNHS GICELHCPALVTYNTDTFESMPNPEGRYTFGASCVTACPYNYLSTDVGSCTLVCPLHNQEVTAEDG TQRCEKCSKPCARVCYGLGMEHLREVRAVTSANIQEFAGCKKIFGSLAFLPESFDG corresponding to amino acids 1-383 of NP_{—}004439 (SEQ ID NO:540), which also corresponds to amino acids 1-383 of S57296_{—}1_P59 (SEQ ID NO:542), a second amino acid sequence being at least 70%, optionally at least 80%, preferably at least 85%, more preferably at least 90% and most preferably at least 95% homologous to a polypeptide having the sequence VSLCQQAGVQWYDLGSLQPLPPGFKQFSCLSLLSSWDYR (SEQ ID NO: 672) corresponding to amino acids 384-422 of S57296_{—}1_P59 (SEQ ID NO:542), and a third amino acid sequence being at least 90% homologous to DPASNTAPLQPEQLQVFETLEEITGYLYISAWPDSLPDLSVFQNLQVIRGRILHNGAYSLTLQGLGIS WLGLRSLRELGSGLALIHHNTHLCFVHTVPWDQLFRNPHQALLHTANRPEDECVGEGLACHQLC ARGHCWGPGPTQCVNCSQFLRGQECVEECRVLQGLPREYVNARHCLPCHPECQPQNGSVTCFGPE ADQCVACAHYKDPPFCVARCPSGVKPDLSYMPIWKFPDEEGACQPCPINCTHSCVDLDDKGCPAE QRASPLTSIISAVVGILLVVVLGVVFGILIKRRQQKIRKYTMRRLLQETELVEPLTPSGAMPNQAQM RILKETELRKVKVLGSGAFGTVYKGIWIPDGENVKIPVAIKVLRENTSPKANKEILDEAYVMAGVG SPYVSRLLGICLTSTVQLVTQLMPYGCLLDHVRENRGRLGSQDLLNWCMQIAKGMSYLEDVRLV HRDLAARNVLVKSPNHVKITDFGLARLLDIDETEYHADGGKVPIKWMALESILRRRFTHQSDVWS YGVTVWELMTFGAKPYDGIPAREIPDLLEKGERLPQPPICTIDVYMIMVKCWMIDSECRPRFRELV SEFSRMARDPQRFVVIQNEDLGPASPLDSTFYRSLLEDDDMGDLVDAEEYLVPQQGFFCPDPAPGA GGMVHHRIIRSSSTRSGGGDLTLGLEPSEEEAPRSPLAPSEGAGSDVFDGDLGMGAAKGLQSLPTH DPSPLQRYSEDPTVPLPSETDGYVAPLTCSPQPEYVNQPDVRPQPPSPREGPLPAARPAGATLERPK TLSPGKNGVVKDVFAFGGAVENPEYLTPQGGAAPQPHPPPAFSPAFDNLYYWDQDPPERGAPPST FKGTPTAENPEYLGLDVPV corresponding to amino acids 384-1255 of NP_{—}004439 (SEQ ID NO:540), which also corresponds to amino acids 423-1294 of S57296_{—}1_P59 (SEQ ID NO:542), wherein said first amino acid sequence, second amino acid sequence and third amino acid sequence are contiguous and in a sequential order.

B. An isolated polypeptide encoding for an edge portion of S57296_{—}1_P59 (SEQ ID NO:542), comprising an amino acid sequence being at least 70%, optionally at least about 80%, preferably at least about 85%, more preferably at least about 90% and most preferably at least about 95% homologous to the sequence VSLCQQAGVQWYDLGSLQPLPPGFKQFSCLSLLSSWDYR (SEQ ID NO: 672) of S572961_P59 (SEQ ID NO:542).

### 3. Comparison Report Between S57296_{—}1_P59 (SEQ ID NO:542) and NP_{—}001005862 (SEQ ID NO:539):

A. An isolated chimeric polypeptide encoding for S57296_{—}1_P59 (SEQ ID NO:542), comprising a first amino acid sequence being at least 70%, optionally at least 80%, preferably at least 85%, more preferably at least 90% and most preferably at least 95%, homologous to a polypeptide having the sequence MELAALCRWGLLLALLPPGAASTQVCTGTD (SEQ ID NO: 673) corresponding to amino acids 1-30 of S57296_{—}1_P59 (SEQ ID NO:542), a second amino acid sequence being at least 70%, optionally at least 80%, preferably at least 85%, more preferably at least 90% and most preferably at least 95% homologous to a polypeptide having the sequence MKLRLPASPETHLDMLRHLYQGCQVVQGNLELTYLPTNA (SEQ ID NO: 674) corresponding to amino acids 384-422 of S57296_{—}1_P59 (SEQ ID NO:542), a third amino acid sequence being at least 90% homologous to SLSFLQDIQEVQGYVLIAHNQVRQVPLQRLRIVRGTQLFEDNYALAVLDNGDPLNNTTPVTGASPG GLRELQLRSLTEILKGGVLIQRNPQLCYQDTILWKDIFHKNNQLALTLIDTNRSRACHPCSPMCKGS RCWGESSEDCQSLTRTVCAGGCARCKGPLPTDCCHEQCAAGCTGPKHSDCLACLHFNHSGICELH CPALVTYNTDTFESMPNPEGRYTFGASCVTACPYNYLSTDVGSCTLVCPLHNQEVTAEDGTQRCE KCSKPCARVCYGLGMEHLREVRAVTSANIQEFAGCKKIFGSLAFLPESFDGDPASNTAPLQPEQLQ VFETLEEITGYLYISAWPDSLPDLSVFQNLQVIRGRILHNGAYSLTLQGLGISWLGLRSLRELGSGLA LIHHNTHLCFVHTVPWDQLFRNPHQALLHTANRPEDECVGEGLACHQLCARGHCWGPGPTQCVN CSQFLRGQECVEECRVLQGLPREYVNARHCLPCHPECQPQNGSVTCFGPEADQCVACAHYKDPPF CVARCPSGVKPDLSYMPIWKFPDEEGACQPCPINCTHSCVDLDDKGCPAEQRASPLTSIISAVVGIL LVVVLGVVFGILIKRRQQKIRKYTMRRLLQETELVEPLTPSGAMPNQAQMRILKETELRKVKVLGS GAFGTVYKGIWIPDGENVKIPVAIKVLRENTSPKANKEILDEAYVMAGVGSPYVSRLLGICLTSTV QLVTQLMPYGCLLDHVRENRGRLGSQDLLNWCMQIAKGMSYLEDVRLVHRDLAARNVLVKSPN HVKITDFGLARLLDIDETEYHADGGKVPIKWMALESILRRRFTHQ corresponding to amino acids 354-1225 of NP_{—}001005862 (SEQ ID NO:539), which also corresponds to amino acids 423-1294 of S57296_{—}1_P59 (SEQ ID NO:542), and a fourth amino acid sequence being at least 90% homologous to SDVWSYGVTVWELMTFGAKPYDGIPAREIPDLLEKGERLPQPPICTIDVYMIMVKCWMIDSECRPR FRELVSEFSRMARDPQRFVVIQNEDLGPASPLDSTFYRSLLEDDDMGDLVDAEEYLVPQQGFFCPD PAPGAGGMVHHRHRSSSTRSGGGDLTLGLEPSEEEAPRSPLAPSEGAGSDVFDGDLGMGAAKGLQ SLPTHDPSPLQRYSEDPTVPLPSETDGYVAPLTCSPQPEYVNQPDVRPQPPSPREGPLPAARPAGAT LERPKTLSPGKNGVVKDVFAFGGAVENPEYLTPQGGAAPQPHPPPAFSPAFDNLYYWDQDPPERG APPSTFKGTPTAENPEYLGLDVPV corresponding to amino acids 1-353 of NP_{—}001005862 (SEQ ID NO:539), which also corresponds to amino acids 31-383 of S57296_{—}1_P59 (SEQ ID NO:542), wherein said first amino acid sequence, second amino acid sequence, third amino acid sequence and fourth amino acid sequence are contiguous and in a sequential order.

B. An isolated polypeptide encoding for a head of S57296_{—}1_P59 (SEQ ID NO:542), comprising a polypeptide being at least 70%, optionally at least about 80%, preferably at least about 85%, more preferably at least about 90% and most preferably at least about 95% homologous to the sequence MELAALCRWGLLLALLPPGAASTQVCTGTD (SEQ ID NO: 673) of S57296_{—}1_P59 (SEQ ID NO:542).

C. An isolated polypeptide encoding for an edge portion of S57296_{—}1_P59 (SEQ ID NO:542), comprising an amino acid sequence being at least 70%, optionally at least about 80%, preferably at least about 85%, more preferably at least about 90% and most preferably at least about 95% homologous to the sequence MKLRLPASPETHLDMLRHLYQGCQVVQGNLELTYLPTNA (SEQ ID NO: 674) of S57296_{—}1_P59 (SEQ ID NO:542).

The location of the variant protein was determined according to results from a number of different software programs and analyses, including analyses from SignalP and other specialized programs. The variant protein is believed to be located as follows with regard to the cell: membrane. The protein localization is believed to be membrane because although both signal-peptide prediction programs agree that this protein has a signal peptide, both trans-membrane region prediction programs predict that this protein has a trans-membrane region downstream of this signal peptide.

Variant protein S57296_{—}1_P59 (SEQ ID NO:542) also has the following non-silent SNPs (Single Nucleotide Polymorphisms) as listed in Table 208, (given according to their position(s) on the amino acid sequence, with the alternative amino acid(s) listed; the last column indicates whether the SNP is known or not; the presence of known SNPs in variant protein S57296_{—}1_P59 (SEQ ID NO:542) sequence provides support for the deduced sequence of this variant protein according to the present invention).

TABLE 208
Amino acid mutations

| SNP position(s) on | Alternative | |
|---|---|---|
| amino acid sequence | amino acid(s) | Previously known SNP? |
| 3 | A -> T | No |
| 105 | Q -> * | No |
| 112 | A -> V | No |
| 131 | A -> G | No |
| 327 | T -> A | No |
| 369 | I -> N | No |
| 465 | N -> D | No |
| 490 | W -> C | Yes |
| 692 | I -> V | Yes |
| 693 | I -> V | Yes |
| 701 | L -> V | No |
| 709 | F -> | No |
| 769 | F -> L | No |
| 793 | L -> W | No |
| 796 | N -> D | No |
| 817 | S -> | No |
| 822 | R -> | No |
| 843 | C -> | No |
| 851 | N -> K | No |
| 852 | R -> | No |
| 868 | A -> | No |
| 885 | A -> | No |
| 885 | A -> G | No |
| 919 | G -> E | No |
| 922 | V -> A | No |
| 965 | P -> R | Yes |
| 1019 | R -> | No |
| 1072 | D -> G | No |
| 1108 | A -> | No |
| 1138 | S -> | No |
| 1158 | L -> | No |
| 1172 | S -> G | No |
| 1193 | G -> | No |
| 1208 | P -> A | Yes |
| 1214 | G -> | No |
| 1238 | Q -> | No |
| 1257 | N -> | No |
| 1264 | D -> Y | No |
| 1277 | G -> | No |

The variant protein has the following domains, as determined by using InterPro. The domains are described in Table 209:

TABLE 209
InterPro domain(s)

| Domain description | Analysis type | Position(s) on protein |
|---|---|---|
| Protein kinase | BlastProDom | 763-1022 |
| Tyrosine protein kinase | FPrintScan | 837-850, 874-892, |
| | | 923-933, 942-964, |
| | | 986-1008 |
| Protein kinase | HMMPfam | 759-1015 |
| Furin-like cysteine rich region | HMMPfam | 189-343 |
| Epidermal growth-factor | HMMPfam | 52-173, 426-525 |
| receptor (EGFR), L domain | | |
| Tyrosine protein kinase | HMMSmart | 759-1015 |
| Serine | HMMSmart | 759-1016 |
| Furin-like repeat | HMMSmart | 232-275, 540-591, |
| | | 596-645 |
| Protein kinase | ProfileScan | 759-1026 |
| Calcium-binding EF-hand | ScanRegExp | 1050-1062 |
| Protein kinase | ScanRegExp | 765-792 |
| Tyrosine protein kinase, active | ScanRegExp | 880-892 |
| site | | |
| Placeholder for matches | Seg | 3-21, 492-506, |
| with an unknown IPR | | 697-714, 1083-1102, |
| | | 1238-1256 |

Variant protein S57296_{—}1_P59 (SEQ ID NO:542) is encoded by the following transcript(s): S57296_{—}1_T78 (SEQ ID NO:481). The coding portion of transcript S57296_{—}1_T78 (SEQ ID NO:481) starts at position 238 and ends at position 4119. The transcript also has the following SNPs as listed in Table 210 (given according to their position on the nucleotide sequence, with the alternative nucleic acid listed; the last column indicates whether the SNP is known or not; the presence of known SNPs in variant protein S57296_{—}1_P59 (SEQ ID NO:542) sequence provides support for the deduced sequence of this variant protein according to the present invention).

TABLE210
Nucleic acid SNPs

| SNP position on nucleotide | Alternative | |
|---|---|---|
| sequence | nucleic acid | Previously known SNP? |
| 155 | -> T | No |
| 160 | C -> T | No |
| 246 | G -> A | No |
| 335 | G -> A | Yes |
| 552 | C -> T | No |
| 574 | C -> T | No |
| 617 | C -> G | Yes |
| 631 | C -> G | No |
| 764 | C -> T | No |
| 1218 | A -> G | No |
| 1345 | T -> A | No |
| 1511 | C -> T | No |
| 1535 | A -> G | No |
| 1632 | A -> G | No |
| 1679 | G -> T | Yes |
| 1709 | G -> T | Yes |
| 1724 | A -> G | Yes |
| 1931 | C -> T | No |
| 2039 | G -> A | Yes |
| 2313 | A -> G | Yes |
| 2316 | A -> G | Yes |
| 2340 | C -> G | No |
| 2345 | C -> A | No |
| 2364 | T -> | No |
| 2531 | T -> G | No |
| 2546 | T -> G | No |
| 2617 | T -> G | No |
| 2625 | A -> G | No |
| 2689 | C -> | No |
| 2705 | C -> G | No |
| 2705 | C -> | No |
| 2768 | C -> | No |
| 2792 | C -> G | No |
| 2795 | C -> G | No |
| 2795 | C -> | No |
| 2842 | C -> | No |
| 2893 | C -> G | No |
| 2893 | C -> | No |
| 2995 | G -> A | No |
| 3004 | T -> C | No |
| 3086 | T -> C | No |
| 3133 | C -> G | Yes |
| 3295 | G -> | No |
| 3317 | C -> T | No |
| 3423 | C -> T | Yes |
| 3431 | C -> T | Yes |
| 3454 | A -> G | No |
| 3515 | T -> C | No |
| 3562 | C -> | No |
| 3652 | G -> | No |
| 3711 | C -> | No |
| 3753 | A -> G | No |
| 3818 | C -> | No |
| 3821 | T -> C | No |
| 3861 | C -> G | Yes |
| 3879 | G -> | No |
| 3884 | G -> A | Yes |
| 3953 | G -> | No |
| 4009 | A -> | No |
| 4029 | G -> T | No |
| 4058 | C -> T | Yes |
| 4070 | G -> | No |
| 4126 | G -> T | Yes |
| 4187 | C -> T | Yes |
| 4196 | A -> | Yes |
| 4226 | G -> | No |
| 4531 | C -> G | No |
| 4531 | C -> | No |
| 4534 | C -> G | Yes |
| 4646 | G -> C | No |
| 4646 | G -> | No |
| 4712 | T -> C | Yes |

Variant protein S57296_{—}1_P65 (SEQ ID NO:543) according to the present invention has an amino acid sequence; it is encoded by transcript(s) S57296_{—}1_T77 (SEQ ID NO:480). An alignment is given to the known protein (Herstatin (SEQ ID NO:534)). One or more alignments to one or more previously published protein sequences are given in the alignment table located on the attached CDROM.

### Comparison Report Between S57296_{—}1_P65 (SEQ ID NO:543) and Q9UK79_HUMAN (SEQ ID NO:534):

A. An isolated chimeric polypeptide encoding for S57296_{—}1_P65 (SEQ ID NO:543), comprising a first amino acid sequence being at least 90% homologous to MELAALCRWGLLLALLPPGAASTQVCTGTDMKLRLPASPETHLDMLRHLYQGCQVVQGNLELTY LPTNASLSFLQDIQEVQGYVLIAHNQVRQVPLQRLRIVRGTQLFEDNYALAVLDNGDPLNNTTPVT GASPGGLRELQLRSLTEILKGGVLIQRNPQLCYQDTILWKDIFHKNNQLALTLIDTNRSRACHPCSP MCKGSRCWGESSEDCQSLTRTVCAGGCARCKGPLPTDCCHEQCAAGCTGPKHSDCLACLHFNHS GICELHCPALVTYNTDTFESMPNPEGRYTFGASCVTACPYNYLSTDVGSCTLVCPLHNQEVTAEDG TQRCEKCSKPCAR corresponding to amino acids 1-340 of Q9UK79_HUMAN (SEQ ID NO:534), which also corresponds to amino acids 1-340 of S57296_{—}1_P65 (SEQ ID NO:543), and a second amino acid sequence being at least 70%, optionally at least 80%, preferably at least 85%, more preferably at least 90% and most preferably at least 95% homologous to a polypeptide having the sequence VCYGLGMEHLREVRAVTSANIQEFAGCKKIFGSLAFLPESFDGDPASNTAPLQPEQLQVFETLEEIT GYLYISAWPDSLPDLSVFQNLQVIRGRILHNGAYSLTLQGLGISWLGLRSLRELGSGLALIHHNTHL CFVHTVPWDQLFRNPHQALLHTANRPEDECGKTGSPVCALPICQHTAVPRGPWQQRSWTCADCP SLCTLLDSAQLWLAWPLGMASLAGSYLPWHPSLPLCF (SEQ ID NO: 675) corresponding to amino acids 341-575 of S57296_{—}1_P65 (SEQ ID NO:543), wherein said first amino acid sequence and second amino acid sequence are contiguous and in a sequential order.

B. An isolated polypeptide encoding for an edge portion of S57296_{—}1_P65 (SEQ ID NO:543), comprising an amino acid sequence being at least 70%, optionally at least about 80%, preferably at least about 85%, more preferably at least about 90% and most preferably at least about 95% homologous to the sequence

(SEQ ID NO:543)

VCYGLGMEHLREVRAVTSANIQEFAGCKKIFGSLAFLPESFDGDPASNTA
PLQPEQLQVFETLEEITGYLYISAWPDSLPDLSVFQNLQVIRGRILHNGA
YSLTLQGLGISWLGLRSLRELGSGLALIHHNTHLCFVHTVPWDQLFRNPH
QALLHTANRPEDECGKTGSPVCALPICQHTAVPRGPWQQRSWTCADCPSL
CTLLDSAQLWLAWPLGMASLAGSYLPWHPSLPLCF
(SEQ ID NO: 675) of S57296_1_P65.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein S57296_{—}1_P65 (SEQ ID NO:543) also has the following non-silent SNPs (Single Nucleotide Polymorphisms) as listed in Table 211, (given according to their position(s) on the amino acid sequence, with the alternative amino acid(s) listed; the last column indicates whether the SNP is known or not; the presence of known SNPs in variant protein S57296_{—}1_P65 (SEQ ID NO:543) sequence provides support for the deduced sequence of this variant protein according to the present invention).

TABLE 211
Amino acid mutations

| SNP position(s) on amino acid | Alternative | |
|---|---|---|
| sequence | amino acid(s) | Previously known SNP? |
| 3 | A -> T | No |
| 105 | Q -> * | No |
| 112 | A -> V | No |
| 131 | A -> G | No |
| 327 | T -> A | No |
| 369 | I -> N | No |
| 426 | N -> D | No |
| 451 | W -> C | Yes |

The variant protein has the following domains, as determined by using InterPro. The domains are described in Table 212:

TABLE 212
InterPro domain(s)

| | | Position(s) |
|---|---|---|
| Domain description | Analysis type | on protein |
| Furin-like cysteine rich region | HMMPfam | 189-343 |
| Epidermal growth-factor receptor | HMMPfam | 52-173, 366-486 |
| (EGFR), L domain | | |
| Furin-like repeat | HMMSmart | 232-275 |
| Placeholder for matches with an | Seg | 3-21, 453-467 |
| unknown IPR | | |

Variant protein S57296_{—}1_P65 (SEQ ID NO:543) is encoded by the following transcript(s): S57296_{—}1T77 (SEQ ID NO:480). The coding portion of transcript S57296_{—}1_T77 (SEQ ID NO:480) starts at position 238 and ends at position 1962. The transcript also has the following SNPs as listed in Table 213 (given according to their position on the nucleotide sequence, with the alternative nucleic acid listed; the last column indicates whether the SNP is known or not; the presence of known SNPs in variant protein S57296_{—}1_P65 (SEQ ID NO:543) sequence provides support for the deduced sequence of this variant protein according to the present invention).

TABLE 213
Nucleic acid SNPs

| SNP position on nucleotide | Alternative | Previously |
|---|---|---|
| sequence | nucleic acid | known SNP? |
| 155 | -> T | No |
| 160 | C -> T | No |
| 246 | G -> A | No |
| 335 | G -> A | Yes |
| 552 | C -> T | No |
| 574 | C -> T | No |
| 617 | C -> G | Yes |
| 631 | C -> G | No |
| 764 | C -> T | No |
| 1218 | A -> G | No |
| 1345 | T -> A | No |
| 1394 | C -> T | No |
| 1418 | A -> G | No |
| 1515 | A -> G | No |
| 1562 | G -> T | Yes |
| 1592 | G -> T | Yes |
| 1607 | A -> G | Yes |

Variant protein S57296_{—}1_P85 (SEQ ID NO:544) according to the present invention has an amino acid sequence; it is encoded by transcript(s) S57296_{—}1_T79 (SEQ ID NO:482). An alignment is given to the known protein (Herstatin (SEQ ID NO:534)). One or more alignments to one or more previously published protein sequences are given in the alignment table located on the attached CDROM.

### Comparison Report Between S57296_{—}1_P85 (SEQ ID NO:544) and Q9UK79_HUMAN (SEQ ID NO:534):

A. An isolated chimeric polypeptide encoding for S57296_{—}1_P85 (SEQ ID NO:544), comprising a first amino acid sequence being at least 90% homologous to MELAALCRWGLLLALLPPGAASTQVCTGTDMKLRLPASPETHLDMLRHLYQGCQVVQGNLELTY LPTNASLSFLQDIQEVQGYVLIAHNQVRQVPLQRLRIVRGTQLFEDNYALAVLDNGDPLNNTTPVT GASPGGLRELQLRSLTEILKGGVLIQRNPQLCYQDTILWKDIFHKNNQLALTLIDTNRSRACHPCSP MCKGSRCWGESSEDCQSLTRTVCAGGCARCKGPLPTDCCHEQCAAGCTGPKHSDCLACLHFNHS GICELHCPALVTYNTDTFESMPNPEGRYTFGASCVTACPYNYLSTDVGSCTLVCPLHNQEVTAEDG TQRCEKCSKPCAR corresponding to amino acids 1-340 of Q9UK79_HUMAN (SEQ ID NO:534), which also corresponds to amino acids 1-340 of S57296_{—}1_P85 (SEQ ID NO:544), and a second amino acid sequence being at least 70%, optionally at least 80%, preferably at least 85%, more preferably at least 90% and most preferably at least 95% homologous to a polypeptide having the sequence VCYGLGMEHLREVRAVTSANIQEFAGCKKIFGSLAFLPESFDGVSLCQQAGVQWYDLGSLQPLPP GFKQFSCLSLLSSWDYRDPASNTAPLQPEQLQVFETLEEITGYLYISAWPDSLPDLSVFQNLQVIRG RILHNGAYSLTLQGLGISWLGLRSLRELGSGLALIHHNTHLCFVHTVPWDQLFRNPHQALLHTANR PEDECGKTGSPVCALPICQHTAVPRGPWQQRSWTCADCPSLCTLLDSAQLWLAWPLGMASLAGS YLPWHPSLPLCF (SEQ ID NO: 676) corresponding to amino acids 341-614 of S57296_{—}1_P85 (SEQ ID NO:544), wherein said first amino acid sequence and second amino acid sequence are contiguous and in a sequential order.

B. An isolated polypeptide encoding for an edge portion of S57296_{—}1_P85 (SEQ ID NO:544), comprising an amino acid sequence being at least 70%, optionally at least about 80%, preferably at least about 85%, more preferably at least about 90% and most preferably at least about 95% homologous to the sequence

(SEQ ID NO:544)

VCYGLGMEHLREVRAVTSANIQEFAGCKKIFGSLAFLPESFDGVSLCQQA
GVQWYDLGSLQPLPPGFKQFSCLSLLSSWDYRDPASNTAPLQPEQLQVFE
TLEEITGYLYISAWPDSLPDLSVFQNLQVIRGRILHNGAYSLTLQGLGIS
WLGLRSLRELGSGLALIHHNTHLCFVHTVPWDQLFRNPHQALLHTANRPE
DECGKTGSPVCALPICQHTAVPRGPWQQRSWTCADCPSLCTLLDSAQLWL
AWPLGMASLAGSYLPWHPSLPLCF (SEQ ID NO: 676) of
S57296_1_P85.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein S57296_{—}1_P85 (SEQ ID NO:544) also has the following non-silent SNPs (Single Nucleotide Polymorphisms) as listed in Table 214, (given according to their position(s) on the amino acid sequence, with the alternative amino acid(s) listed; the last column indicates whether the SNP is known or not; the presence of known SNPs in variant protein S57296_{—}1_P85 (SEQ ID NO:544) sequence provides support for the deduced sequence of this variant protein according to the present invention).

TABLE 214
Amino acid mutations

| SNP position(s) on amino acid | Alternative | |
|---|---|---|
| sequence | amino acid(s) | Previously known SNP? |
| 3 | A -> T | No |
| 105 | Q -> * | No |
| 112 | A -> V | No |
| 131 | A -> G | No |
| 327 | T -> A | No |
| 369 | I -> N | No |
| 465 | N -> D | No |
| 490 | W -> C | Yes |

The variant protein has the following domains, as determined by using InterPro. The domains are described in Table 215:

TABLE 215
InterPro domain(s)

| | | Position(s) |
|---|---|---|
| Domain description | Analysis type | on protein |
| Furin-like cysteine rich region | HMMPfam | 189-343 |
| Epidermal growth-factor receptor | HMMPfam | 52-173, 426-525 |
| (EGFR), L domain | | |
| Furin-like repeat | HMMSmart | 232-275 |
| Placeholder for matches with an | Seg | 3-21, 492-506 |
| unknown IPR | | |

Variant protein S57296_{—}1_P85 (SEQ ID NO:544) is encoded by the following transcript(s): S57296_{—}1_T79 (SEQ ID NO:482). The coding portion of transcript S57296_{—}1_T79 (SEQ ID NO:482) starts at position 238 and ends at position 2079. The transcript also has the following SNPs as listed in Table 216 (given according to their position on the nucleotide sequence, with the alternative nucleic acid listed; the last column indicates whether the SNP is known or not; the presence of known SNPs in variant protein S57296_{—}1_P85 (SEQ ID NO:544) sequence provides support for the deduced sequence of this variant protein according to the present invention).

TABLE 216
Nucleic acid SNPs

| SNP position on | | |
|---|---|---|
| nucleotide sequence | Alternative nucleic acid | Previously known SNP? |
| 155 | -> T | No |
| 160 | C -> T | No |
| 246 | G -> A | No |
| 335 | G -> A | Yes |
| 552 | C -> T | No |
| 574 | C -> T | No |
| 617 | C -> G | Yes |
| 631 | C -> G | No |
| 764 | C -> T | No |
| 1218 | A -> G | No |
| 1345 | T -> A | No |
| 1511 | C -> T | No |
| 1535 | A -> G | No |
| 1632 | A -> G | No |
| 1679 | G -> T | Yes |
| 1709 | G -> T | Yes |
| 1724 | A -> G | Yes |

Variant protein S57296_{—}1_P97 (SEQ ID NO:545) according to the present invention has an amino acid sequence; it is encoded by transcript(s) S57296_{—}1_T80 (SEQ ID NO:483). An alignment is given to the known protein (Herstatin (SEQ ID NO:534)). One or more alignments to one or more previously published protein sequences are in the alignment table located on the attached CDROM.

### Comparison Report Between S57296_{—}1_P97 (SEQ ID NO:545) and Q9UK79_HUMAN (SEQ ID NO:534):

A. An isolated chimeric polypeptide encoding for S57296_{—}1_P97 (SEQ ID NO:545), comprising a first amino acid sequence being at least 90% homologous to MELAALCRWGLLLALLPPGAASTQVCTGTDMKLRLPASPETHLDMLRHLYQGCQVVQGNLELTY LPTNASLSFLQDIQEVQGYVLIAHNQVRQVPLQRLRIVRGTQLFEDNYALAVLDNGDPLNNTTPVT GASPGGLRELQLRSLTEILKGGVLIQRNPQLCYQDTILWKDIFHKNNQLALTLIDTNRSRACHPCSP MCKGSRCWGESSEDCQSLTRTVCAGGCARCKGPLPTDCCHEQCAAGCTGPKHSDCLACLHFNHS GICELHCPALVTYNTDTFESMPNPEGRYTFGASCVTACPYNYLSTDVGSCTLVCPLHNQEVTAEDG TQRCEKCSKPCARGT corresponding to amino acids 1-342 of Q9UK79_HUMAN (SEQ ID NO:534), which also corresponds to amino acids 1-342 of S57296_{—}1_P97 (SEQ ID NO:545), and a second amino acid sequence being at least 70%, optionally at least 80%, preferably at least 85%, more preferably at least 90% and most preferably at least 95% homologous to a polypeptide having the sequence QPPTLPRSSQSSSKCLRLWKRSQVTYTSQHGRTACLTSASSRTCK (SEQ ID NO: 677) corresponding to amino acids 343-387 of S57296_{—}1_P97 (SEQ ID NO:545), wherein said first amino acid sequence and second amino acid sequence are contiguous and in a sequential order.

B. An isolated polypeptide encoding for an edge portion of S57296_{—}1_P97 (SEQ ID NO:545), comprising an amino acid sequence being at least 70%, optionally at least about 80%, preferably at least about 85%, more preferably at least about 90% and most preferably at least about 95% homologous to the sequence QPPTLPRSSQSSSKCLRLWKRSQVTYTSQHGRTACLTSASSRTCK (SEQ ID NO: 677) of S57296_{—}1_P97 (SEQ ID NO:545).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein S57296_{—}1_P97 (SEQ ID NO:545) also has the following non-silent SNPs (Single Nucleotide Polymorphisms) as listed in Table 217, (given according to their position(s) on the amino acid sequence, with the alternative amino acid(s) listed; the last column indicates whether the SNP is known or not; the presence of known SNPs in variant protein S57296_{—}1_P97 (SEQ ID NO:545) sequence provides support for the deduced sequence of this variant protein according to the present invention).

TABLE 217
Amino acid mutations

| SNP position(s) on | Alternative | Previously |
|---|---|---|
| amino acid sequence | amino acid(s) | known SNP? |
| 3 | A -> T | No |
| 105 | Q -> * | No |
| 112 | A -> V | No |
| 131 | A -> G | No |
| 327 | T -> A | No |
| 343 | P -> L | No |
| 351 | Q -> R | No |

The variant protein has the following domains, as determined by using InterPro. The domains are described in Table 218:

TABLE 218
InterPro domain(s)

| | | Position(s) |
|---|---|---|
| Domain description | Analysis type | on protein |
| Furin-like cysteine rich region | HMMPfam | 189-340 |
| Epidermal growth-factor receptor | HMMPfam | 52-173 |
| (EGFR), L domain | | |
| Furin-like repeat | HMMSmart | 232-275 |
| Placeholder for matches with an | Seg | 3-21 |
| unknown IPR | | |

Variant protein S57296_{—}1_P97 (SEQ ID NO:545) is encoded by the following transcript(s): S57296_{—}1_T80 (SEQ ID NO:483). The coding portion of transcript S57296_{—}1_T80 (SEQ ID NO:483) starts at position 238 and ends at position 1398. The transcript also has the following SNPs as listed in Table 219 (given according to their position on the nucleotide sequence, with the alternative nucleic acid listed; the last column indicates whether the SNP is known or not; the presence of known SNPs in variant protein S57296_{—}1_P97 (SEQ ID NO:545) sequence provides support for the deduced sequence of this variant protein according to the present invention).

TABLE 219
Nucleic acid SNPs

| SNP position on | | |
|---|---|---|
| nucleotide sequence | Alternative nucleic acid | Previously known SNP? |
| 155 | -> T | No |
| 160 | C -> T | No |
| 246 | G -> A | No |
| 335 | G -> A | Yes |
| 552 | C -> T | No |
| 574 | C -> T | No |
| 617 | C -> G | Yes |
| 631 | C -> G | No |
| 764 | C -> T | No |
| 1218 | A -> G | No |
| 1267 | C -> T | No |
| 1291 | A -> G | No |
| 1388 | A -> G | No |

Variant protein S57296_{—}1_P125 (SEQ ID NO:546) according to the present invention has an amino acid sequence; it is encoded by transcript(s) S57296_{—}1_T75 (SEQ ID NO:478). An alignment is given to the known protein (Herstatin (SEQ ID NO:534)). One or more alignments to one or more previously published protein sequences in the alignment table located on the attached CDROM.

### 1. Comparison Report Between S57296_{—}1_P125 (SEQ ID NO:546) and ERB2_HUMAN (SEQ ID NO:538):

A. An isolated chimeric polypeptide encoding for S57296_{—}1_P125 (SEQ ID NO:546), comprising a first amino acid sequence being at least 90% homologous to MELAALCRWGLLLALLPPGAASTQVCTGTDMKLRLPASPETHLDMLRHLYQGCQVVQGNLELTY LPTNASLSFLQDIQEVQGYVLIAHNQVRQVPLQRLRIVRGTQLFEDNYALAVLDNGDPLNNTTPVT GASPGGLRELQLRSLTEILKGGVLIQRNPQLCYQDTILWKDIFHKNNQLALTLIDTNRSRACHPCSP MCKGSRCWGESSEDCQSLTRTVCAGGCARCKGPLPTDCCHEQCAAGCTGPKHSDCLACLHFNHS GICELHCPALVTYNTDTFESMPNPEGRYTFGASCVTACPYNYLSTDVGSCTLVCPLHNQEVTAEDG TQRCEKCSKPCARVCYGLGMEHLREVRAVTSANIQEFAGCKKIFGSLAFLPESFDGDPASNTAPLQ PEQLQVFETLEEITGYLYISAWPDSLPDLSVFQNLQVIRGRILHNGAYSLTLQGLGISWLGLRSLREL GSGLALIHHNTHLCFVHTVPWDQLFRNPHQALLHTANRPEDECVGEGLACHQLCARGHCWGPGP TQCVNCSQFLRGQECVEECRVLQGLPREYVNARHCLPCHPECQPQNGSVTCFGPEADQCVACAH YKDPPFCVARCPSGVKPDLSYMPIWKFPDEEGACQPCPINCTHSCVDLDDKGCPAEQRA corresponding to amino acids 1-648 of ERB2_HUMAN (SEQ ID NO:538), which also corresponds to amino acids 1-648 of S57296_{—}1_P125 (SEQ ID NO:546), and a second amino acid sequence being at least 70%, optionally at least 80%, preferably at least 85%, more preferably at least 90% and most preferably at least 95% homologous to a polypeptide having the sequence RLAWTPGCTLHCPSLPHWMLGGHCCREGTP (SEQ ID NO: 678) corresponding to amino acids 649-678 of S57296_{—}1_P125 (SEQ ID NO:546), wherein said first amino acid sequence and second amino acid sequence are contiguous and in a sequential order.

B. An isolated polypeptide encoding for an edge portion of S57296_{—}1_P125 (SEQ ID NO:546), comprising an amino acid sequence being at least 70%, optionally at least about 80%, preferably at least about 85%, more preferably at least about 90% and most preferably at least about 95% homologous to the sequence RLAWTPGCTLHCPSLPHWMLGGHCCREGTP (SEQ ID NO: 678) of S57296_{—}1_P125 (SEQ ID NO:546).

### 2. Comparison Report Between S57296_{—}1_P125 (SEQ ID NO:546) and NP_{—}004439 (SEQ ID NO:540):

A. An isolated chimeric polypeptide encoding for S57296_{—}1_P125 (SEQ ID NO:546), comprising a first amino acid sequence being at least 90% homologous to MELAALCRWGLLLALLPPGAASTQVCTGTDMKLRLPASPETHLDMLRHLYQGCQVVQGNLELTY LPTNASLSFLQDIQEVQGYVLIAHNQVRQVPLQRLRIVRGTQLFEDNYALAVLDNGDPLNNTTPVT GASPGGLRELQLRSLTEILKGGVLIQRNPQLCYQDTILWKDIFHKNNQLALTLIDTNRSRACHPCSP MCKGSRCWGESSEDCQSLTRTVCAGGCARCKGPLPTDCCHEQCAAGCTGPKHSDCLACLHFNHS GICELHCPALVTYNTDTFESMPNPEGRYTFGASCVTACPYNYLSTDVGSCTLVCPLHNQEVTAEDG TQRCEKCSKPCARVCYGLGMEHLREVRAVTSANIQEFAGCKKIFGSLAFLPESFDGDPASNTAPLQ PEQLQVFETLEEITGYLYISAWPDSLPDLSVFQNLQVIRGRILHNGAYSLTLQGLGISWLGLRSLREL GSGLALIHHNTHLCFVHTVPWDQLFRNPHQALLHTANRPEDECVGEGLACHQLCARGHCWGPGP TQCVNCSQFLRGQECVEECRVLQGLPREYVNARHCLPCHPECQPQNGSVTCFGPEADQCVACAH YKDPPFCVARCPSGVKPDLSYMPIWKFPDEEGACQPCPINCTHSCVDLDDKGCPAEQRA corresponding to amino acids 1-648 of NP_{—}004439 (SEQ ID NO:540), which also corresponds to amino acids 1-648 of S57296_{—}1_P125 (SEQ ID NO:546), and a second amino acid sequence being at least 70%, optionally at least 80%, preferably at least 85%, more preferably at least 90% and most preferably at least 95% homologous to a polypeptide having the sequence RLAWTPGCTLHCPSLPHWMLGGHCCREGTP (SEQ ID NO: 678) corresponding to amino acids 649-678 of S57296_{—}1_P125 (SEQ ID NO:546), wherein said first amino acid sequence and second amino acid sequence are contiguous and in a sequential order.

B. An isolated polypeptide encoding for an edge portion of S57296_{—}1_P125 (SEQ ID NO:546), comprising an amino acid sequence being at least 70%, optionally at least about 80%, preferably at least about 85%, more preferably at least about 90% and most preferably at least about 95% homologous to the sequence RLAWTPGCTLHCPSLPHWMLGGHCCREGTP (SEQ ID NO: 678) of S57296_{—}1_P125 (SEQ ID NO:546).

### 3. Comparison Report Between S57296_{—}1_P125 (SEQ ID NO:546) and NP_{—}001005862 (SEQ ID NO:539):

A. An isolated chimeric polypeptide encoding for S57296_{—}1_P125 (SEQ ID NO:546), comprising a first amino acid sequence being at least 70%, optionally at least 80%, preferably at least 85%, more preferably at least 90% and most preferably at least 95%, homologous to a polypeptide having the sequence MELAALCRWGLLLALLPPGAASTQVCTGTD (SEQ ID NO: 673) corresponding to amino acids 1-30 of S57296_{—}1_P125 (SEQ ID NO:546), a second amino acid sequence being at least 70%, optionally at least 80%, preferably at least 85%, more preferably at least 90% and most preferably at least 95%, homologous to a polypeptide having the sequence MKLRLPASPETHLDMLRHLYQGCQVVQGNLELTYLPTNASLSFLQDIQEVQGYVLIAHNQVRQVP LQRLRIVRGTQLFEDNYALAVLDNGDPLNNTTPVTGASPGGLRELQLRSLTEILKGGVLIQRNPQL CYQDTILWKDIFHKNNQLALTLIDTNRSRACHPCSPMCKGSRCWGESSEDCQSLTRTVCAGGCAR CKGPLPTDCCHEQCAAGCTGPKHSDCLACLHFNHSGICELHCPALVTYNTDTFESMPNPEGRYTFG ASCVTACPYNYLSTDVGSCTLVCPLHNQEVTAEDGTQRCEKCSKPCARVCYGLGMEHLREVRAV TSANIQEFAGCKKIFGSLAFLPESFDGDPASNTAPLQPEQLQVFETLEEITGYLYISAWPDSLPDLSV FQNLQVIRGRILHNGAYSLTLQGLGISWLGLRSLRELGSGLALIHHNTHLCFVHTVPWDQLFRNPH QALLHTANRPEDECVGEGLACHQLCARGHCWGPGPTQCVNCSQFLRGQECVEECRVLQGLPREY VNARHCLPCHPECQPQNGSVTCFGPEADQCVACAHYKDPPFCVARCPSGVKPDLSYMPIWKFPDE EGACQPCPINCTHSCVDLDDKGCPAEQRARLAWTPGCTLHCPSLPHWM corresponding to amino acids 649-678 of S57296_{—}1_P125 (SEQ ID NO:546), and a third amino acid sequence being at least 90% homologous to SPLTSIISAVVGILLVVVLGVVFGILIKRRQQKIRKYTMRRLLQETELVEPLTPSGAMPNQAQMRILK ETELRKVKVLGSGAFGTVYKGIWIPDGENVKIPVAIKVLRENTSPKANKEILDEAYVMAGVGSPYV SRLLGICLTSTVQLVTQLMPYGCLLDHVRENRGRLGSQDLLNWCMQIAKGMSYLEDVRLVHRDL AARNVLVKSPNHVKITDFGLARLLDIDETEYHADGGKVPIKWMALESILRRRFTHQSDVWSYGVT VWELMTFGAKPYDGIPAREIPDLLEKGERLPQPPICTIDVYMIMVKCWMIDSECRPRFRELVSEFSR MARDPQRFVVIQNEDLGPASPLDSTFYRSLLEDDDMGDLVDAEEYLVPQQGFFCPDPAPGAGGM VHHRHRSSSTRSGGGDLTLGLEPSEEEAPRSPLAPSEGAGSDVFDGDLGMGAAKGLQSLPTHDPSP LQRYSEDPTVPLPSETDGYVAPLTCSPQPEYVNQPDVRPQPPSPREGPLPAARPAGATLERPKTLSP GKNGVVKDVFAFGGAVENPEYLTPQGGAAPQPHPPPAFSPAFDNLYYWDQDPPERGAPPSTFKGT PTAENPEYLGLDVPV corresponding to amino acids 1-618 of NP_{—}001005862 (SEQ ID NO:539), which also corresponds to amino acids 31-648 of S57296_{—}1_P125 (SEQ ID NO:546), wherein said first amino acid sequence, second amino acid sequence and third amino acid sequence are contiguous and in a sequential order.

B. An isolated polypeptide encoding for a head of S57296_{—}1_P125 (SEQ ID NO:546), comprising a polypeptide being at least 70%, optionally at least about 80%, preferably at least about 85%, more preferably at least about 90% and most preferably at least about 95% homologous to the sequence MELAALCRWGLLLALLPPGAASTQVCTGTD (SEQ ID NO: 673) of S57296_{—}1_P125 (SEQ ID NO:546).

### 4. Comparison Report Between S57296_{—}1_P125 (SEQ ID NO:546) and Q9UK79_HUMAN (SEQ ID NO:534):

A. An isolated chimeric polypeptide encoding for S57296_{—}1_P125 (SEQ ID NO:546), comprising a first amino acid sequence being at least 90% homologous to MELAALCRWGLLLALLPPGAASTQVCTGTDMKLRLPASPETHLDMLRHLYQGCQVVQGNLELTY LPTNASLSFLQDIQEVQGYVLIAHNQVRQVPLQRLRIVRGTQLFEDNYALAVLDNGDPLNNTTPVT GASPGGLRELQLRSLTEILKGGVLIQRNPQLCYQDTILWKDIFHKNNQLALTLIDTNRSRACHPCSP MCKGSRCWGESSEDCQSLTRTVCAGGCARCKGPLPTDCCHEQCAAGCTGPKHSDCLACLHFNHS GICELHCPALVTYNTDTFESMPNPEGRYTFGASCVTACPYNYLSTDVGSCTLVCPLHNQEVTAEDG TQRCEKCSKPCAR corresponding to amino acids 1-340 of Q9UK79_HUMAN (SEQ ID NO:534), which also corresponds to amino acids 1-340 of S57296_{—}1_P125 (SEQ ID NO:546), and a second amino acid sequence being at least 70%, optionally at least 80%, preferably at least 85%, more preferably at least 90% and most preferably at least 95% homologous to a polypeptide having the sequence VCYGLGMEHLREVRAVTSANIQEFAGCKKIFGSLAFLPESFDGDPASNTAPLQPEQLQVFETLEEIT GYLYISAWPDSLPDLSVFQNLQVIRGRILHNGAYSLTLQGLGISWLGLRSLRELGSGLALIHHNTHL CFVHTVPWDQLFRNPHQALLHTANRPEDECVGEGLACHQLCARGHCWGPGPTQCVNCSQFLRGQ ECVEECRVLQGLPREYVNARHCLPCHPECQPQNGSVTCFGPEADQCVACAHYKDPPFCVARCPSG VKPDLSYMPIWKFPDEEGACQPCPINCTHSCVDLDDKGCPAEQRARLAWTPGCTLHCPSLPHWML GGHCCREGTP corresponding to amino acids 341-678 of S57296_{—}1_P125 (SEQ ID NO:546), wherein said first amino acid sequence and second amino acid sequence are contiguous and in a sequential order.

B. An isolated polypeptide encoding for an edge portion of S57296_{—}1_P125 (SEQ ID NO:546), comprising an amino acid sequence being at least 70%, optionally at least about 80%, preferably at least about 85%, more preferably at least about 90% and most preferably at least about 95% homologous to the sequence

| | |
|---|---|
| VCYGLGMEHLREVRAVTSANIQEFAGCKKIFGSLAFLPESFDGDPASNTAPLQPEQLQVFETLEEIT | (SEQ ID NO: 680) |
| GYLYISAWPDSLPDLSVFQNLQVIRGRILHNGAYSLTLQGLGISWLGLRSLRELGSGLALIHHNTHL | of S57296_1_P125 |
| CFVHTVPWDQLFRNPHQALLHTANRPEDECVGEGLACHQLCARGHCWGPGPTQCVNCSQFLRGQ | (SEQ ID NO: 546) |
| ECVEECRVLQGLPREYVNARHCLPCHPECQPQNGSVTCFGPEADQCVACAHYKDPPFCVARCPSG | |
| VKPDLSYMPIWKFPDEEGACQPCPINCTHSCVDLDDKGCPAEQRARLAWTPGCTLHCPSLPHWML | |
| GGHCCREGTP. | |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein S57296_{—}1_P125 (SEQ ID NO:546) also has the following non-silent SNPs (Single Nucleotide Polymorphisms) as listed in Table 220, (given according to their position(s) on the amino acid sequence, with the alternative amino acid(s) listed; the last column indicates whether the SNP is known or not; the presence of known SNPs in variant protein S57296_{—}1_P125 (SEQ ID NO:546) sequence provides support for the deduced sequence of this variant protein according to the present invention).

TABLE 220
Amino acid mutations

| SNP position(s) on | | |
|---|---|---|
| amino acid sequence | Alternative amino acid(s) | Previously known SNP? |
| 3 | A -> T | No |
| 105 | Q -> * | No |
| 112 | A -> V | No |
| 131 | A -> G | No |
| 327 | T -> A | No |
| 369 | I -> N | No |
| 426 | N -> D | No |
| 451 | W -> C | Yes |

The variant protein has the following domains, as determined by using InterPro. The domains are described in Table 221:

TABLE 221
InterPro domain(s)

| Domain description | Analysis type | Position(s) on protein |
|---|---|---|
| 4Fe-4S ferredoxin, iron-sulfur | FPrintScan | 577-588, 595-606 |
| binding | | |
| Furin-like cysteine rich region | HMMPfam | 189-343 |
| Epidermal growth-factor | HMMPfam | 52-173, 366-486 |
| receptor (EGFR), L domain | | |
| Furin-like repeat | HMMSmart | 232-275, 501-552, |
| | | 557-606 |
| Placeholder for matches with an | Seg | 3-21, 453-467 |
| unknown IPR | | |

Variant protein S57296_{—}1_P125 (SEQ ID NO:546) is encoded by the following transcript(s): S57296_{—}1_T75 (SEQ ID NO:478). The coding portion of transcript S57296_{—}1_T75 (SEQ ID NO:478) starts at position 238 and ends at position 2271. The transcript also has the following SNPs as listed in Table 222 (given according to their position on the nucleotide sequence, with the alternative nucleic acid listed; the last column indicates whether the SNP is known or not; the presence of known SNPs in variant protein S57296_{—}1_P125 (SEQ ID NO:546) sequence provides support for the deduced sequence of this variant protein according to the present invention).

TABLE 222
Nucleic acid SNPs

| SNP position on | | |
|---|---|---|
| nucleotide sequence | Alternative nucleic acid | Previously known SNP? |
| 155 | -> T | No |
| 160 | C -> T | No |
| 246 | G -> A | No |
| 335 | G -> A | Yes |
| 552 | C -> T | No |
| 574 | C -> T | No |
| 617 | C -> G | Yes |
| 631 | C -> G | No |
| 764 | C -> T | No |
| 1218 | A -> G | No |
| 1345 | T -> A | No |
| 1394 | C -> T | No |
| 1418 | A -> G | No |
| 1515 | A -> G | No |
| 1562 | G -> T | Yes |
| 1592 | G -> T | Yes |
| 1607 | A -> G | Yes |
| 1814 | C -> T | No |
| 1922 | G -> A | Yes |
| 2274 | C -> G | Yes |
| 2281 | C -> G | Yes |
| 2474 | G -> C | Yes |

Variant protein S57296_{—}1_P127 (SEQ ID NO:547) according to the present invention has an amino acid sequence; it is encoded by transcript(s) S57296_{—}1_T76 (SEQ ID NO:479). An alignment is given to the known protein (Herstatin (SEQ ID NO:534)). One or more alignments to one or more previously published protein sequences are given in the alignment table located on the attached CDROM.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein S57296_{—}1_P127 (SEQ ID NO:547) also has the following non-silent SNPs (Single Nucleotide Polymorphisms) as listed in Table 223, (given according to their position(s) on the amino acid sequence, with the alternative amino acid(s) listed; the last column indicates whether the SNP is known or not; the presence of known SNPs in variant protein S57296_{—}1_P127 (SEQ ID NO:547) sequence provides support for the deduced sequence of this variant protein according to the present invention).

TABLE 223
Amino acid mutations

| SNP position(s) on | | |
|---|---|---|
| amino acid sequence | Alternative amino acid(s) | Previously known SNP? |
| 3 | A -> T | No |
| 105 | Q -> * | No |
| 112 | A -> V | No |
| 131 | A -> G | No |
| 327 | T -> A | No |
| 369 | I -> N | No |
| 465 | N -> D | No |
| 490 | W -> C | Yes |

The variant protein has the following domains, as determined by using InterPro. The domains are described in Table 224:

TABLE 224
InterPro domain(s)

| Domain description | Analysis type | Position(s) on protein |
|---|---|---|
| 4Fe-4S ferredoxin, iron-sulfur | FPrintScan | 616-627, 634-645 |
| binding | | |
| Furin-like cysteine rich region | HMMPfam | 189-343 |
| Epidermal growth-factor | HMMPfam | 52-173, 426-525 |
| receptor (EGFR), L domain | | |
| Furin-like repeat | HMMSmart | 232-275, 540-591, |
| | | 596-645 |
| Placeholder for matches with an | Seg | 3-21, 492-506 |
| unknown IPR | | |

Variant protein S57296_{—}1_P127 (SEQ ID NO:547) is encoded by the following transcript(s): S57296_{—}1_T76 (SEQ ID NO:479). The coding portion of transcript S57296_{—}1_T76 (SEQ ID NO:479) starts at position 238 and ends at position 2388. The transcript also has the following SNPs as listed in Table 225 (given according to their position on the nucleotide sequence, with the alternative nucleic acid listed; the last column indicates whether the SNP is known or not; the presence of known SNPs in variant protein S57296_{—}1_P127 (SEQ ID NO:547) sequence provides support for the deduced sequence of this variant protein according to the present invention).

TABLE 225
Nucleic acid SNPs

| SNP position on | | |
|---|---|---|
| nucleotide sequence | Alternative nucleic acid | Previously known SNP? |
| 155 | -> T | No |
| 160 | C -> T | No |
| 246 | G -> A | No |
| 335 | G -> A | Yes |
| 552 | C -> T | No |
| 574 | C -> T | No |
| 617 | C -> G | Yes |
| 631 | C -> G | No |
| 764 | C -> T | No |
| 1218 | A -> G | No |
| 1345 | T -> A | No |
| 1511 | C -> T | No |
| 1535 | A -> G | No |
| 1632 | A -> G | No |
| 1679 | G -> T | Yes |
| 1709 | G -> T | Yes |
| 1724 | A -> G | Yes |
| 1931 | C -> T | No |
| 2039 | G -> A | Yes |
| 2391 | C -> G | Yes |
| 2398 | C -> G | Yes |
| 2591 | G -> C | Yes |

Table 226 below describes the starting and ending position of S57296_{—}1_N59 (SEQ ID NO:495) on each of the relevant transcripts. Experimental results for this segment are described below.

TABLE 226
Segment location on transcripts

| | Segment | Segment |
|---|---|---|
| Transcript name | starting position | ending position |
| S57296_1_T75 (SEQ ID NO: 478) | 2184 | 2637 |
| S57296_1_T76 (SEQ ID NO: 479) | 2301 | 2747 |

Table 227 below describes the starting and ending position of S57296_{—}1_N58 (SEQ ID NO:510) on each of the relevant transcripts. Experimental results for this segment are described below.

TABLE 227
Segment location on transcripts

| | Segment | Segment |
|---|---|---|
| Transcript name | starting position | ending position |
| S57296_1_T75 (SEQ ID NO: 478) | 2136 | 2183 |
| S57296_1_T76 (SEQ ID NO: 479) | 2253 | 2300 |
| S57296_1_T78 (SEQ ID NO: 481) | 2253 | 2300 |

Table 228 below describes the starting and ending position of S57296_{—}1_N63 (SEQ ID NO:511) on the relevant transcript. Experimental results for this segment are described below.

TABLE 228
Segment location on transcripts

| | Segment | Segment |
|---|---|---|
| Transcript name | starting position | ending position |
| S57296_1_T78 (SEQ ID NO: 481) | 2440 | 2520 |

The starting and ending positions of S57296_{—}1_N46 (SEQ ID NO:552) on transcript S57296_{—}1_T77 (SEQ ID NO: 480) are 1751 and 2031 respectively.

The starting and ending positions of S57296_{—}1_N46 (SEQ ID NO:552) on transcript S57296_{—}1_T79 (SEQ ID NO:482) are 1868 and 2148 respectively.

Expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296-B2Lnew seg58-59 (SEQ ID NO: 550) in normal and cancerous breast tissues

Expression of Homo sapiens v-erb-b2 erythroblastic leukemia viral oncogene homolog 2, neuro/glioblastoma derived oncogene homolog (avian) (ERBB2) transcripts detectable by or according to seg58-59-by S57296-B2Lnew seg58-59 (SEQ ID NO: 550) amplicon and primers S57296 seg58-59F (SEQ ID NO: 548) and S57296 seg58-59R (SEQ ID NO: 549) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and G6PD (GenBank Accession No. NM_{—}000402 (SEQ ID NO:8); G6PD amplicon (SEQ ID NO: 44)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) and post surgery (PS) samples (Sample Nos. 56-60, 63-67, Table 6, “Tissue samples in breast cancer testing panel” above), to obtain a value of fold up-regulation for each sample relative to median of the normal PM samples.

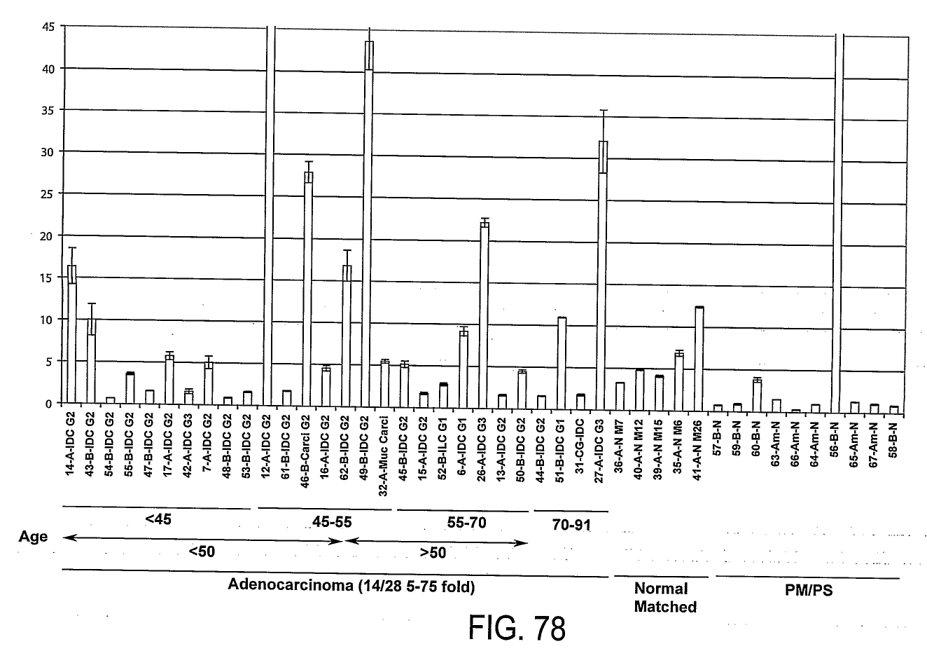

FIG. 78 is a histogram showing over expression of the above-indicated Homo sapiens v-erb-b2 erythroblastic leukemia viral oncogene homolog 2, neuro/glioblastoma derived oncogene homolog (avian) (ERBB2) transcripts in cancerous breast samples relative to the normal samples. Values represent the average of duplicate experiments. Error bars indicate the minimal and maximal values obtained.

As is evident from FIG. 78, the expression of Homo sapiens v-erb-b2 erythroblastic leukemia viral oncogene homolog 2, neuro/glioblastoma derived oncogene homolog (avian) (ERBB2) transcripts detectable by the above amplicon in cancer samples was significantly higher than in the non-cancerous samples (Sample Nos. 56-60, 63-67 Table 6, “Tissue samples in breast cancer testing panel” above). Notably an over-expression of at least 5 fold was found in 14 out of 28 adenocarcinoma samples.

Statistical analysis was applied to verify the significance of these results, as described below.

Threshold of 5 fold overexpression was found to differentiate between cancer and normal samples with P value of 2.82E-02 as checked by exact fisher test. The above value demonstrate statistical significance of the results.

By dissecting the patient's population into four age groups (indicated below the graph), we demonstrate that the transcripts detected by the above amplicon are highly expressed mainly in the 45-55 age group.

One of the normal samples (Sample No. 56, Table 5, “Tissue samples in breast cancer testing panel” above) showed high over-expression of all checked ErbB-2 transcripts. Since over-expression of ErbB-2 WT was observed in this sample, and also over-expression of hTERT which is known to be expressed only in cancerous cells (data not shown), it is thought that this sample is either contaminated with cancerous cells, or alternatively may have been marked by mistake as a normal sample.

The B2L variants were over expressed at least 5 fold also in 2 of the 5 matched normal samples. Since matched samples are histologically non-cancerous tissue that surrounds the tumor, such samples could have been contaminated with cancer or pre-cancer cells.

Primer pairs are also optionally and preferably encompassed within the present invention; for example, for the above experiment, the following primer pair was used as a non-limiting illustrative example only of a suitable primer pair: S57296 seg58-59F (SEQ ID NO: 548) forward primer; and S57296 seg58-59R (SEQ ID NO: 549) reverse primer.

The present invention also preferably encompasses any amplicon obtained through the use of any suitable primer pair; for example, for the above experiment, the following amplicon was obtained as a non-limiting illustrative example only of a suitable amplicon: S57296 seg58-59 (SEQ ID NO: 550).

Primers:

| | |
|---|---|
| Forward primer S57296 seg58-59F: | (SEQ ID NO: 548) |
| GACAAGGGCTGCCCCG | |
| Reverse primer S57296 seg58-59R: | (SEQ ID NO: 549) |
| CAGTGACCACCCAGCATCC | |
| Amplicon S57296-B2Lnew seg58-59: | (SEQ ID NO: 550) |

GACAAGGGCTGCCCCGCCGAGCAGAGAGCCAGGTTGGCCTGGACCCCAGGATGTACCCTTCA
TTGCCCTTCACTCCCCCACTGGATGCTGGGTGGTCACTG

Expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296 B2S seg-44 (SEQ ID NO: 552), S57296 B2Lnew seg58-59 (SEQ ID NO: 550), S57296WT seg63 (SEQ ID NO:497) in normal and cancerous lung tissues

Expression of Homo sapiens v-erb-b2 erythroblastic leukemia viral oncogene homolog 2, neuro/glioblastoma derived oncogene homolog (avian) (ERBB2) transcripts detectable by or according to S57296 B2S seg-44 (SEQ ID NO: 552), S57296 B2Lnew seg58-59 (SEQ ID NO: 550), S57296WT seg63 (SEQ ID NO:497) amplicons and primers S57296 B2S seg-44F (SEQ ID NO: 551), seg58-59F (SEQ ID NO: 548), seg63F (SEQ ID NO: 555) and S57296 seg-44R, seg58-59R (SEQ ID NO: 549), seg63R (SEQ ID NO: 556) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and Ubiquitin (GenBank Accession No. BC000449 (SEQ ID NO:9); amplicon—Ubiquitin-amplicon (SEQ ID NO:50)) was measured similarly. For each RT sample, the expression of the above amplicons was normalized to the geometric mean of the quantities of the housekeeping genes. For each amplicon, the normalized quantity of each RT sample was then divided by the median of the quantities of the normal post-mortem (PM) samples (Sample Nos. 47-50, 90-93, 96-99, Table 5, above, “Tissue samples in lung cancer testing panel”), to obtain a value of fold differential expression for each sample relative to median of the normal PM samples.

In one experiment that was carried out no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pairs: S57296 B2S seg-44F (SEQ ID NO: 551), seg58-59F (SEQ ID NO: 548), seg63F (SEQ ID NO: 555) forward primers; and S57296 seg44R, seg58-59R (SEQ ID NO: 549), seg63R (SEQ ID NO: 556) reverse primers.

The present invention also preferably encompasses any amplicon obtained through the use of any suitable primer pair; for example, for the above experiment, the following amplicon was obtained as a non-limiting illustrative example only of a suitable amplicons: S57296 B2S seg-44 (SEQ ID NO: 552), S57296 B2Lnew seg58-59 (SEQ ID NO: 550), S57296WT seg63 (SEQ ID NO:497)

Primers:

| Forward primer S57296 B2S seg-44 F: | (SEQ ID NO: 551) |
|---|---|
| CAGCGTTCTTGGACTTGTGC | |
| Reverse primer S57296 B2S seg-44 R: | (SEQ ID NO: 671) |
| CCAGCTAGAGAAGCCATGCC | |
| Amplicon S57296 B2S seg-44: | (SEQ ID NO: 552) |
| CAGCGTTCTTGGACTTGTGCAGACTGCCCGTCTCTGTGCACCCTTCTTGACTCAGCACAGCTCT | |
| GGCTGGCTTGGCCTCTTGGCATGGCTTCTCTAGCTGG | |
| Forward primer S57296 seg58-59 F: | (SEQ ID NO: 548) |
| GACAAGGGCTGCCCCG | |
| Reverse primer S57296 seg58-59 R: | (SEQ ID NO: 549) |
| CAGTGACCACCCAGCATCC | |
| Amplicon S57296 seg58-59: | (SEQ ID NO: 550) |
| GACAAGGGCTGCCCCGCCGAGCAGAGAGCCAGGTTGGCCTGGACCCCAGGATGTACCCTTCA | |
| TTGCCCTTCACTCCCCCACTGGATGCTGGGTGGTCACTG | |
| Forward primer S57296-WT seg63 F: | (SEQ ID NO: 553) |
| CCCATATGTCTCCCGCCTT | |
| Reverse primer S57296-WT seg63 R: | (SEQ ID NO: 554) |
| ACATGGTCTAAGAGGCAGCCA | |
| Amplicon S57296 seg63: | (SEQ ID NO: 557) |

CCCATATGTCTCCCGCCTTCTGGGCATCTGCCTGACATCCACGGTGCAGCTGGTGACACAGCTT
ATGCCCTATGGCTGCCTCTTAGACCATGT

Expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296 B2S seg-44 (SEQ ID NO: 552), S57296 B2Lnew seg58-59 (SEQ ID NO: 550), S57296WT seg63 (SEQ ID NO:497) in different normal tissues:

Expression of Homo sapiens v-erb-b2 erythroblastic leukemia viral oncogene homolog 2, neuro/glioblastoma derived oncogene homolog (avian) (ERBB2) transcripts detectable by or according to S57296 B2S seg-44 (SEQ ID NO: 552), S57296 B2Lnew seg58-59 (SEQ ID NO: 550), S57296WT seg63 (SEQ ID NO:497) amplicons and primers: S57296 B2S seg-44F (SEQ ID NO: 551), seg58-59F (SEQ ID NO: 548), seg63F (SEQ ID NO: 555) and S57296 seg44R, seg58-59R (SEQ ID NO: 549), seg63R (SEQ ID NO: 556) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)) was measured similarly. For each RT sample, the expression of the above amplicons was normalized to the geometric mean of the quantities of the housekeeping genes. For each amplicon, the normalized quantity of each RT sample was then divided by the median of the quantities of the breast samples (Sample Nos. 33-35 Table 7, “Tissue samples in normal panel”), to obtain a value of relative expression of each sample relative to median of the breast samples.

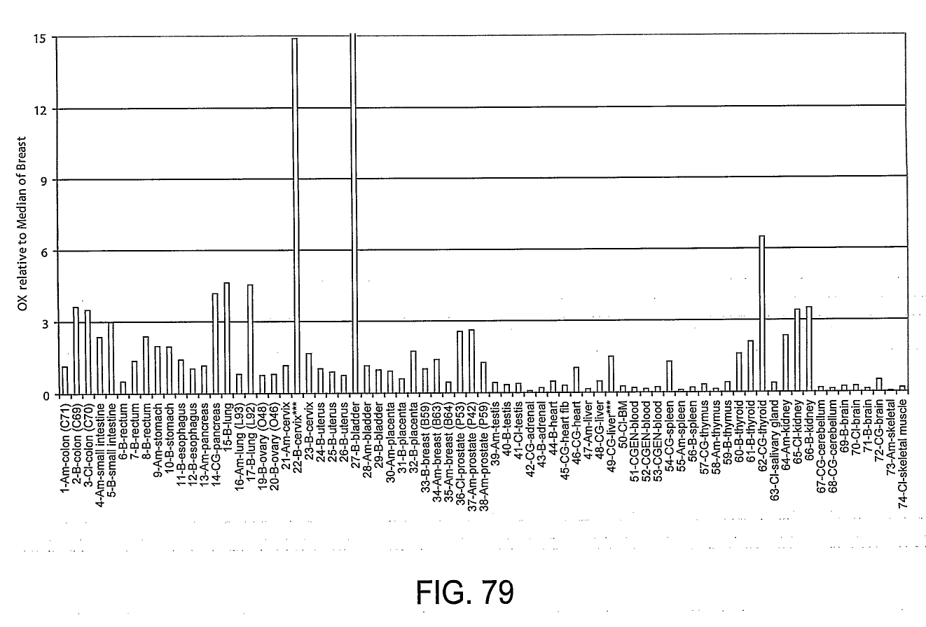

FIG. 79 is a histogram showing expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296 B2S seg-44 (SEQ ID NO: 552) in different normal tissues.

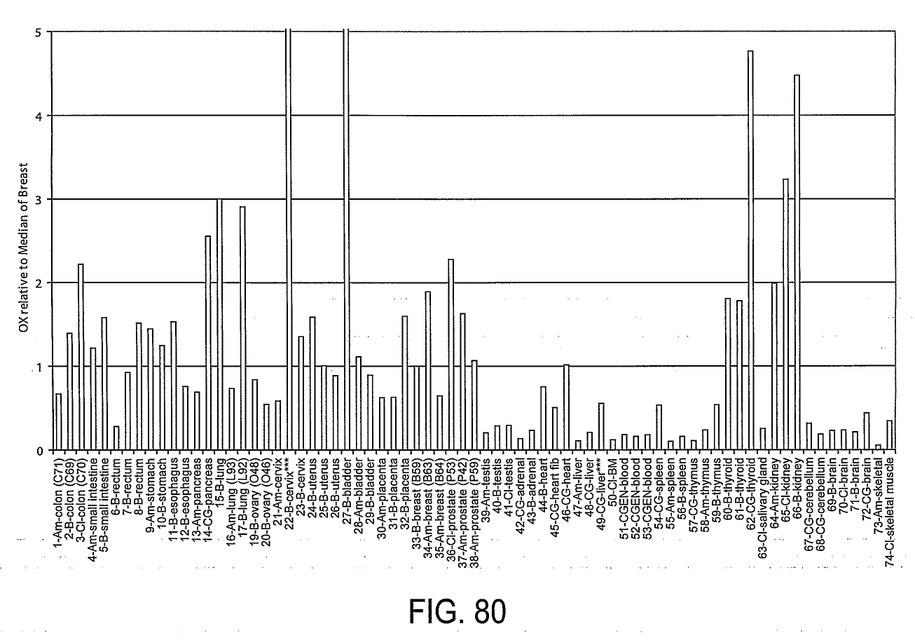

FIG. 80 is a histogram showing expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296 B2Lnew seg58-59 (SEQ ID NO: 550) in different normal tissues.

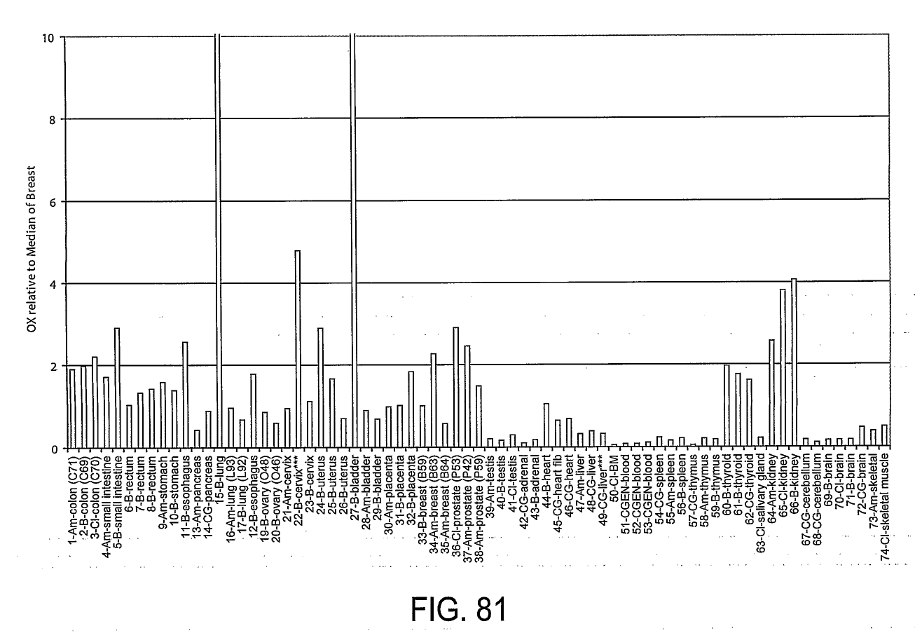

FIG. 81 is a histogram showing expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296WT seg63 (SEQ ID NO:497) in different normal tissues.

Primers:

| Forward primer S57296 B2S seg44 F: | (SEQ ID NO: 551) |
|---|---|
| CAGCGTTCTTGGACTTGTGC | |
| Reverse primer S57296 B2S seg44 R: | |
| CCAGCTAGAGAAGCCATGCC | |
| Amplicon S57296 B2S seg44: | (SEQ ID NO: 552) |
| CAGCGTTCTTGGACTTGTGCAGACTGCCCGTCTCTGTGCACCCTTCTTGACTCAGCACAGCTCT | |
| GGCTGGCTTGGCCTCTTGGCATGGCTTCTCTAGCTGG | |
| Forward primer S57296 B2Lnew seg58-59 F: | (SEQ ID NO: 548) |
| GACAAGGGCTGCCCCG | |
| Reverse primer S57296 B2Lnew seg58-59 R: | |
| CAGTGACCACCCAGCATCC | |
| Amplicon S57296-B2Lnew seg58-59: | (SEQ ID NO: 550) |
| GACAAGGGCTGCCCCGCCGAGCAGAGAGCCAGGTTGGCCTGGACCCCAGGATGTACC | |
| CTTCATTGCCCTTCACTCCCCCACTGGATGCTGGGTGGTCACTG | |
| Forward primer S57296 seg63F: | (SEQ ID NO: 555) |
| CCCATATGTCTCCCGCCTT | |
| Reverse primer S57296 seg63R: | (SEQ ID NO: 556) |
| ACATGGTCTAAGAGGCAGCCA | |
| Amplicon S57296 seg63: | (SEQ ID NO: 557) |

CCCATATGTCTCCCGCCTTCTGGGCATCTGCCTGACATCCACGGTGCAGCTGGTGACA
CAGCTTATGCCCTATGGCTGCCTCTTAGACCATGT

Expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296 B2S seg-44 (SEQ ID NO: 552), S57296 B2Lnew seg58-59 (SEQ ID NO: 550), S57296WT seg63 (SEQ ID NO:497) in normal and cancerous ovary tissues:

Expression of Homo sapiens v-erb-b2 erythroblastic leukemia viral oncogene homolog 2, neuro/glioblastoma derived oncogene homolog (avian) (ERBB2) transcripts detectable by or according to S57296 B2S seg-44 (SEQ ID NO: 552), S57296 B2Lnew seg58-59 (SEQ ID NO: 550), S57296WT seg63 (SEQ ID NO:497) amplicons and primers S57296 B2S seg-44F (SEQ ID NO: 551), seg58-59F (SEQ ID NO: 548), seg63F (SEQ ID NO: 555) and S57296 seg-44R, seg58-59R (SEQ ID NO: 549), seg63R (SEQ ID NO: 556) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and GAPDH (GenBank Accession No. BC026907 (SEQ ID NO:3); GAPDH amplicon (SEQ ID NO: 41) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold differential expression for each sample relative to median of the normal PM samples.

In one experiment that was carried out no differential expression in the cancerous samples relative to the normal PM samples was observed. Primer pairs are also optionally and preferably encompassed within the present invention; for example, for the above experiment, the following primer pair was used as a non-limiting illustrative example only of a suitable primer pairs: S57296 B2S seg-44F (SEQ ID NO: 551), seg58-59F (SEQ ID NO: 548), seg63F (SEQ ID NO: 555) forward primers; and S57296 seg-44R, seg58-59R (SEQ ID NO: 549), seg63R (SEQ ID NO: 556) reverse primers. The present invention also preferably encompasses any amplicon obtained through the use of any suitable primer pair; for example, for the above experiment, the following amplicon was obtained as a non-limiting illustrative example only of a suitable amplicons: S57296 B2S seg-44 (SEQ ID NO: 552), S57296 B2Lnew seg58-59 (SEQ ID NO: 550), S57296-WT seg63

Primers:

| Forward primer S57296 B2S seg-44 F: | (SEQ ID NO: 551) |
|---|---|
| CAGCGTTCTTGGACTTGTGC | |
| Reverse primer S57296 B2S seg-44 R: | |
| CCAGCTAGAGAAGCCATGCC | |
| Amplicon S57296 B2S seg-44: | (SEQ ID NO: 552) |
| CAGCGTTCTTGGACTTGTGCAGACTGCCCGTCTCTGTGCACCCTTCTTGACTCAGCACAGCTCT | |
| GGCTGGCTTGGCCTCTTGGCATGGCTTCTCTAGCTGG | |
| Forward primer S57296 seg58-59 F: | (SEQ ID NO: 548) |
| GACAAGGGCTGCCCCG | |
| Reverse primer S57296 seg58-59 R: | (SEQ ID NO: 549) |
| CAGTGACCACCCAGCATCC | |
| Amplicon S57296 B2Lnew seg58-59: | (SEQ ID NO: 550) |
| GACAAGGGCTGCCCCGCCGAGCAGAGAGCCAGGTTGGCCTGGACCCCAGGATGTACCCTTCA | |
| TTGCCCTTCACTCCCCCACTGGATGCTGGGTGGTCACTG | |
| Forward primer S57296-WT seg63 F: | (SEQ ID NO: 553) |
| CCCATATGTCTCCCGCCTT | |
| Reverse primer S57296-WT seg63 R: | (SEQ ID NO: 554) |
| ACATGGTCTAAGAGGCAGCCA | |
| Amplicon S57296 WT seg63: | |

CCCATATGTCTCCCGCCTTCTGGGCATCTGCCTGACATCCACGGTGCAGCTGGTGACACAGCTT
ATGCCCTATGGCTGCCTCTTAGACCATGT

Expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296 B2S seg-44 (SEQ ID NO: 552), S57296 B2Lnew seg58-59 (SEQ ID NO: 550) in normal and cancerous colon tissues

Expression of Homo sapiens v-erb-b2 erythroblastic leukemia viral oncogene homolog 2, neuro/glioblastoma derived oncogene homolog (avian) (ERBB2) transcripts detectable by or according to S57296 B2S seg-44 (SEQ ID NO: 552), S57296 B2Lnew seg58-59 (SEQ ID NO: 550), and primers S57296 B2S seg-44F (SEQ ID NO: 551), seg58-59F (SEQ ID NO: 548) and S57296 seg-44R, seg58-59R (SEQ ID NO: 549) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), G6PD (GenBank Accession No. NM_{—}000402 (SEQ ID NO:8); G6PD amplicon (SEQ ID NO: 44)) and RPS27A (GenBank Accession No. NM_{—}002954 (SEQ ID NO:1); RPS27A amplicon (SEQ ID NO: 47)), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 4, above, “Tissue samples in colon cancer testing panel”), to obtain a value of fold up-regulation for each sample relative to median of the normal PM samples.

In one experiment that was carried out with each of the above amplicons no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pairs: S57296 B2S seg-44F (SEQ ID NO: 551), seg58-59F (SEQ ID NO: 548), forward primers; and S57296 seg-44R, seg58-59R (SEQ ID NO: 549) reverse primers.

The present invention also preferably encompasses any amplicon obtained through the use of any suitable primer pair; for example, for the above experiment, the following amplicon was obtained as a non-limiting illustrative example only of a suitable amplicons: S57296 B2S seg-44 (SEQ ID NO: 552), S57296 B2Lnew seg58-59 (SEQ ID NO: 550), S57296-WT seg63

Primers:

| Forward primer S57296 B2S seg-44 F: | (SEQ ID NO: 551) |
|---|---|
| CAGCGTTCTTGGACTTGTGC | |
| Reverse primer S57296 B2S seg-44 R: | |
| CCAGCTAGAGAAGCCATGCC | |
| Amplicon S57296 B2S seg-44: | (SEQ ID NO: 552) |
| CAGCGTTCTTGGACTTGTGCAGACTGCCCGTCTCTGTGCACCCTTCTTGACTCAGCACAGCTCT | |
| GGCTGGCTTGGCCTCTTGGCATGGCTTCTCTAGCTGG | |
| Forward primer S57296 seg58-59 F: | (SEQ ID NO: 548) |
| GACAAGGGCTGCCCCG | |
| Reverse primer S57296 seg58-59 R: | (SEQ ID NO: 549) |
| CAGTGACCACCCAGCATCC | |
| Amplicon S57296 B2Lnew seg58-59: | (SEQ ID NO: 550) |

GACAAGGGCTGCCCCGCCGAGCAGAGAGCCAGGTTGGCCTGGACCCCAGGATGTACCCTTCA
TTGCCCTTCACTCCCCCACTGGATGCTGGGTGGTCACTG

Expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296WT seg63 (SEQ ID NO:497) in normal and cancerous breast tissues

Expression of Homo sapiens v-erb-b2 erythroblastic leukemia viral oncogene homolog 2, neuro/glioblastoma derived oncogene homolog (avian) (ERBB2) transcripts detectable by or according to seg63-by S57296WT seg63 (SEQ ID NO:497) amplicon and primers S57296 seg63F (SEQ ID NO: 555) and S57296 seg63R (SEQ ID NO: 556) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and G6PD (GenBank Accession No. NM_{—}000402 (SEQ ID NO:8); G6PD amplicon (SEQ ID NO: 44)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) and post surgery (PS) samples (Sample Nos. 56-60, 63-67, Table 6, above, “Tissue samples in breast cancer testing panel”), to obtain a value of fold up-regulation for each sample relative to median of the normal PM samples.

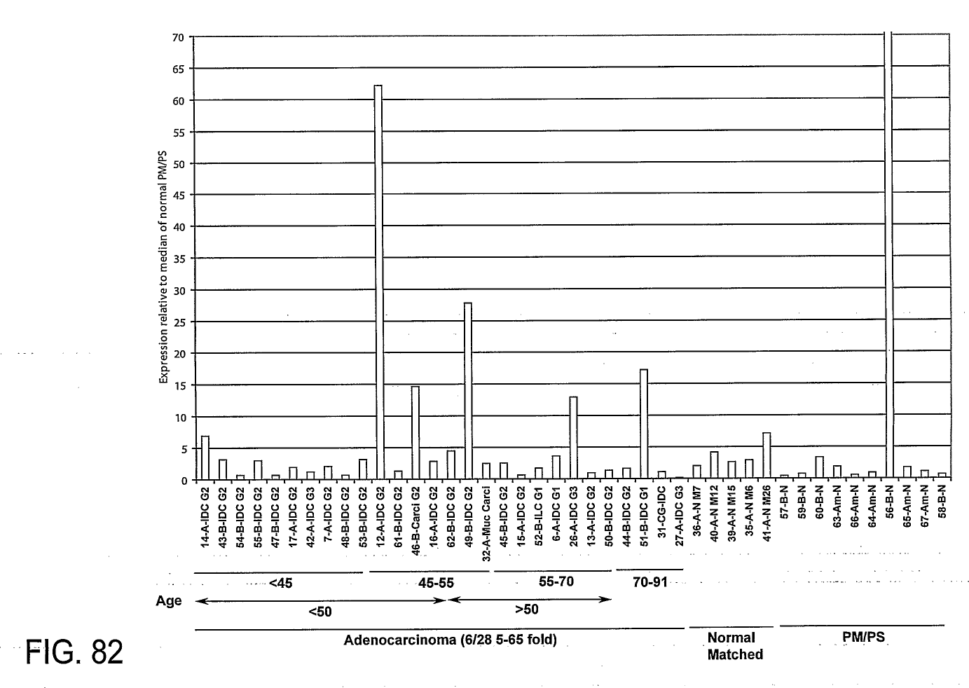

FIG. 82 is a histogram showing over expression of the above-indicated Homo sapiens v-erb-b2 erythroblastic leukemia viral oncogene homolog 2, neuro/glioblastoma derived oncogene homolog (avian) (ERBB2) transcripts in cancerous breast samples relative to the normal samples.

As is evident from FIG. 82, the expression of Homo sapiens v-erb-b2 erythroblastic leukemia viral oncogene homolog 2, neuro/glioblastoma derived oncogene homolog (avian) (ERBB2) transcripts detectable by the above amplicon in cancer samples was significantly higher than in the non-cancerous samples (Sample Nos. 56-60, 63-67 Table 6, “Tissue samples in breast cancer testing panel”). Notably an over-expression of at least 5 fold was found in 6 out of 28 adenocarcinoma samples.

By dissecting the patient's population into four age groups (indicated below the graph), we demonstrate that the transcripts detected by the above amplicon are highly expressed mainly in the 45-55 age group.

One of the normal samples (Sample No. 56, Table 6, above, “Tissue samples in breast cancer testing panel”) showed high over-expression of all checked ErbB-2 transcripts. Since over-expression of ErbB-2 WT was observed in this sample, and also over-expression of hTERT which is known to be expressed only in cancerous cells (data not shown), it is thought that this sample is either contaminated with cancerous cells, or alternatively may have been marked by mistake as a normal sample.

ErbB2 WT was over expressed at least 5 fold also in 1 of the 5 matched normal samples. Since matched samples are histologically non-cancerous tissue that surrounds the tumor, such samples could have been contaminated with cancer or pre-cancer cells.

Primer pairs are also optionally and preferably encompassed within the present invention; for example, for the above experiment, the following primer pair was used as a non-limiting illustrative example only of a suitable primer pair: S57296 seg63F (SEQ ID NO: 555) forward primer; and S57296 seg63R (SEQ ID NO: 556) reverse primer.

The present invention also preferably encompasses any amplicon obtained through the use of any suitable primer pair; for example, for the above experiment, the following amplicon was obtained as a non-limiting illustrative example only of a suitable amplicon: S57296 seg63 (SEQ ID NO:557).

Primers:

| | |
|---|---|
| Forward primer S57296-WT seg63 F: | (SEQ ID NO: 553) |
| CCCATATGTCTCCCGCCTT | |
| Reverse primer S57296-WT seg63 R: | (SEQ ID NO: 554) |
| ACATGGTCTAAGAGGCAGCCA | |
| Amplicon S57296 seg63: | (SEQ ID NO: 557) |

CCCATATGTCTCCCGCCTTCTGGGCATCTGCCTGACATCCACGGTGCAGCTGGTGACACAGCTT
ATGCCCTATGGCTGCCTCTTAGACCATGT

Expression of Homo sapiens v-erb-b2 erythroblastic leukemia viral oncogene homolog 2, neuro/glioblastoma derived oncogene homolog (avian) (ERBB2) S57296 transcripts which are detectable by amplicon as depicted in sequence name S57296 B2S seg-44 (SEQ ID NO: 552) in normal and cancerous breast tissues

Expression of Homo sapiens v-erb-b2 erythroblastic leukemia viral oncogene homolog 2, neuro/glioblastoma derived oncogene homolog (avian) (ERBB2) transcripts detectable by or according to seg-44-by S57296 B2S seg-44 (SEQ ID NO: 552) amplicon and primers S57296 seg44F and S57296 seg44R was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)) and G6PD (GenBank Accession No. NM_{—}000402 (SEQ ID NO:8); G6PD amplicon (SEQ ID NO: 44)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) and post surgery (PS) samples (Sample Nos. 56-60, 63-67, Table 6, above, “Tissue samples in breast cancer testing panel”), to obtain a value of fold up-regulation for each sample relative to median of the normal PM samples.

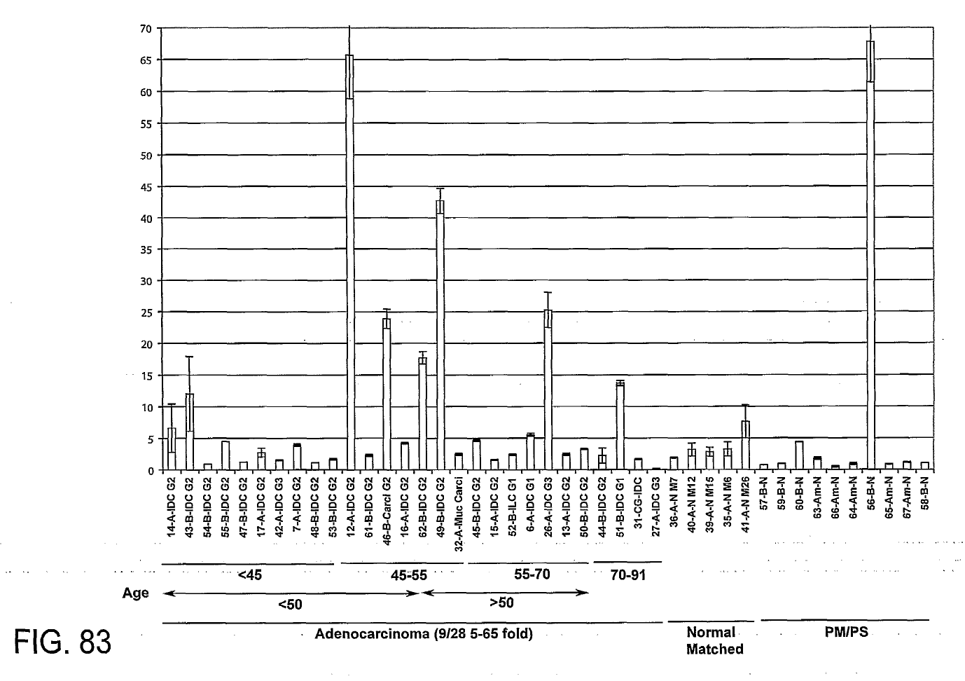

FIG. 83 is a histogram showing over expression of the above-indicated Homo sapiens v-erb-b2 erythroblastic leukemia viral oncogene homolog 2, neuro/glioblastoma derived oncogene homolog (avian) (ERBB2) transcripts in cancerous breast samples relative to the normal samples. Values represent the average of duplicate experiments. Error bars indicate the minimal and maximal values obtained.

As is evident from FIG. 83, the expression of Homo sapiens v-erb-b2 erythroblastic leukemia viral oncogene homolog 2, neuro/glioblastoma derived oncogene homolog (avian) (ERBB2) transcripts detectable by the above amplicon in cancer samples was significantly higher than in the non-cancerous samples (Sample Nos. 56-60, 63-67 Table 6 “Tissue samples in breast cancer testing panel”). Notably an over-expression of at least 5 fold was found in 9 out of 28 adenocarcinoma samples.

By dissecting the patient's population into four age groups (indicated below the graph), we demonstrate that the transcripts detected by the above amplicon are highly expressed mainly in the 45-55 age group.

One of the normal samples (Sample No. 56, Table 6, above, “Tissue samples in breast cancer testing panel”) showed high over-expression of all checked ErbB-2 transcripts. Since over-expression of ErbB-2 WT was observed in this sample, and also over-expression of hTERT which is known to be expressed only in cancerous cells (data not shown), it is thought that this sample is either contaminated with cancerous cells, or alternatively may have been marked by mistake as a normal sample.

The B2S variants were over expressed at least 5 fold also in I of the 5 matched normal samples. Since matched samples are histologically non-cancerous tissue that surrounds the tumor, such samples could have been contaminated with cancer or pre-cancer cells.

Primer pairs are also optionally and preferably encompassed within the present invention; for example, for the above experiment, the following primer pair was used as a non-limiting illustrative example only of a suitable primer pair: S57296 seg44F forward primer; and S57296 seg44R reverse primer.

The present invention also preferably encompasses any amplicon obtained through the use of any suitable primer pair; for example, for the above experiment, the following amplicon was obtained as a non-limiting illustrative example only of a suitable amplicon: S57296 seg44 (SEQ ID NO:552).

Primers:

| | |
|---|---|
| Forward primer S57296 B2S seg-44 F: | (SEQ ID NO: 551) |
| CAGCGTTCTTGGACTTGTGC | |
| Reverse primer S57296 B2S seg-44 R: | |
| CCAGCTAGAGAAGCCATGCC | |
| Amplicon S57296 B2S seg-44: | (SEQ ID NO: 552) |

CAGCGTTCTTGGACTTGTGCAGACTGCCCGTCTCTGTGCACCCTTCTTGACTCAGCACAGCTCT
GGCTGGCTTGGCCTCTTGGCATGGCTTCTCTAGCTGG

Combined expression of 4 sequences—S57296 B2S seg-44 (SEQ ID NO: 552), S57296 B2Lnew seg58-59 (SEQ ID NO: 550), HSSTROL seg20-21 (SEQ ID NO: 560), T94936 seg14 (SEQ ID NO: 563) in normal and cancerous breast tissues.

Expression of v-erb-b2 erythroblastic leukemia viral oncogene homolog 2, neuro/glioblastoma derived oncogene homolog (avian) (ERBB2), Homo sapiens matrix metalloproteinase 11 (stromelysin 3) (MMP11), Homo sapiens breast cancer membrane protein 11 (BCMP11) transcripts detectable by or according to S57296 B2S seg-44 (SEQ ID NO: 552), S57296 B2Lnew seg58-59 (SEQ ID NO: 550), HSSTROL seg20-21 (SEQ ID NO: 560), T94936 seg14 (SEQ ID NO: 563) amplicons and primers—S57296 B2S seg-44F (SEQ ID NO: 551) and S57296 seg-44R; seg58-59F (SEQ ID NO: 548) and seg58-59R (SEQ ID NO: 549); HSSTROL seg20-21F (SEQ ID NO:558) and HSSTROL seg20-21R (SEQ ID NO:559); T94936 seg14F (SEQ ID NO:561) and T94936 seg14R (SEQ ID NO:562) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), G6PD (GenBank Accession No. NM_{—}000402 (SEQ ID NO:8); G6PD amplicon (SEQ ID NO: 44)), was measured similarly. For each RT sample, the expression of the above amplicons was normalized to the geometric mean of the quantities of the housekeeping genes. The normalized quantity of each RT sample of each amplicon was then divided by the median of the quantities of the normal post-mortem (PM) samples detected for the same amplicon (Sample Nos. 56-60, 63-67, Table 6, above, “Tissue samples in breast cancer testing panel”), to obtain a value of fold up-regulation for each sample relative to median of the normal PM samples.

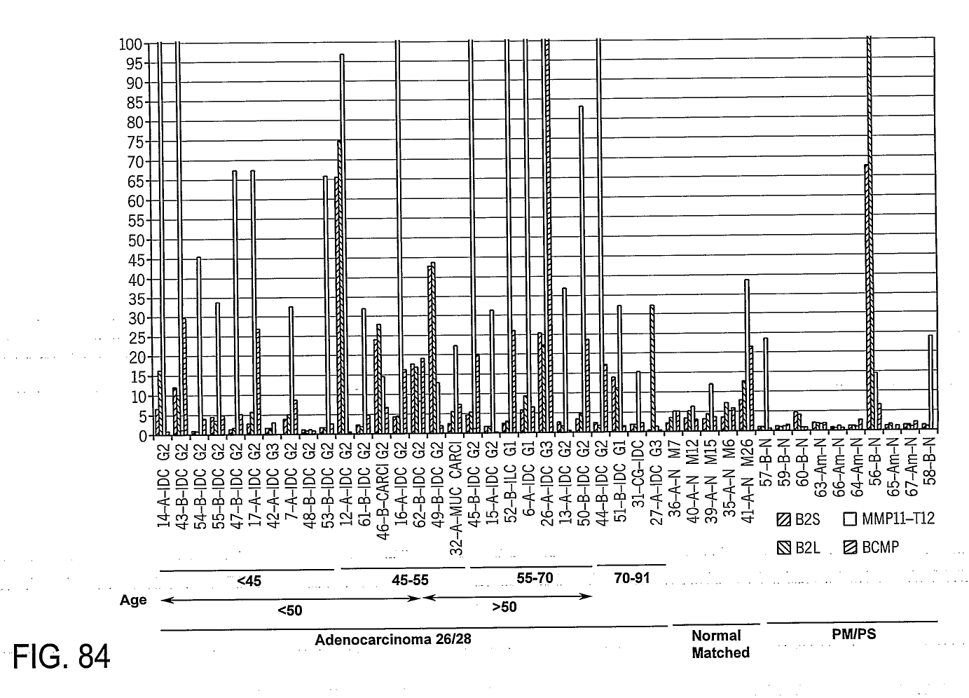

FIG. 84 is a histogram showing differential expression of the above-indicated transcripts in cancerous breast samples relative to the normal samples. The number and percentage of samples that exhibit at least 5 fold differential of at least one of the sequences, out of the total number of samples tested is indicated in the bottom.

As is evident from FIG. 84, differential expression of at least 5 fold in at least one of the sequences was found in 26 out of 28 adenocarcinoma samples.

Primers:

| Forward primer S57296 B2S seg-44 F: | (SEQ ID NO: 551) |
|---|---|
| CAGCGTTCTTGGACTTGTGC | |
| Reverse primer S57296 B2S seg-44 R: | |
| CCAGCTAGAGAAGCCATGCC | |
| Amplicon S57296 B2S seg-44: | (SEQ ID NO: 552) |
| CAGCGTTCTTGGACTTGTGCAGACTGCCCGTCTCTGTGCACCCTTCTTGACTCAGCACAGCTCT | |
| GGCTGGCTTGGCCTCTTGGCATGGCTTCTCTAGCTGG | |
| Forward primer S57296 seg58-59 F: | (SEQ ID NO: 548) |
| GACAAGGGCTGCCCCG | |
| Reverse primer S57296 seg58-59 R: | (SEQ ID NO: 549) |
| CAGTGACCACCCAGCATCC | |
| Amplicon S57296 B2Lnew seg58-59: | (SEQ ID NO: 550) |
| GACAAGGGCTGCCCCGCCGAGCAGAGAGCCAGGTTGGCCTGGACCCCAGGATGTACCCTTCA | |
| TTGCCCTTCACTCCCCCACTGGATGCTGGGTGGTCACTG | |
| Forward primer HSSTROL seg20-21F: | (SEQ ID NO: 558) |
| TCTGCTGGCCACTGTGACTG | |
| Reverse primer HSSTROL seg20-21R: | (SEQ ID NO: 559) |
| GAAGAAAAAGAGCTCGCCTCG | |
| Amplicon HSSTROL seg20-21: | (SEQ ID NO: 560) |
| TCTGCTGGCCACTGTGACTGCAGCATATGCCCTCAGCATGTGTCCCTCTCTCCCACCCCAGCCA | |
| GACGCCCCGCCAGATGCCTGTGAGGCCTCCTTTGACGCGGTCTCCACCATCCGAGGCGAGCTC | |
| TTTTTCTTC | |
| Forward primer T94936 seg14F: | (SEQ ID NO: 561) |
| TACAAAATTAGTAGAAATCAGCATTCTTGC | |
| Reverse primer T94936 seg14R: | (SEQ ID NO: 562) |
| TGTAGAACTAACAAGAGCTGATATTATTGGAT | |
| Amplicon T94936 seg14: | (SEQ ID NO: 563) |

TACAAAATTAGTAGAAATCAGCATTCTTGCTTTTATTTTTAAATGCTAGTTCAAGTACTATTCT
TTTTAAAGAGAAGTCATTTCTAATCCAATAATATCAGCTCTTGTTAGTTCTACA

### Description for Cluster Z36249

Cluster Z36249 features 4 transcript(s) and 11 segment(s) of interest, the names for which are given in Tables 229 and 230, respectively. The selected protein variants are given in table 231.

TABLE 229
Transcripts of interest
Transcript Name

Z36249_PEA_3_T2 (SEQ ID NO: 564)
Z36249_PEA_3_T3 (SEQ ID NO: 565)
Z36249_PEA_3_T5 (SEQ ID NO: 566)
Z36249_PEA_3_T9 (SEQ ID NO: 567)

TABLE 230
Segments of interest

Segment Name

| | |
|---|---|
| Z36249_PEA_3_node_0 (SEQ ID NO: 568) | Z36249_PEA_3_T2 (SEQ ID NO: 564), |
| | Z36249_PEA_3_T3 (SEQ ID NO: 565), |
| | Z36249_PEA_3_T5 (SEQ ID NO: 566) and |
| | Z36249_PEA_3_T9 (SEQ ID NO: 567) |
| Z36249_PEA_3_node_3 (SEQ ID NO: 569) | Z36249_PEA_3_T2 (SEQ ID NO: 564), |
| | Z36249_PEA_3_T3 (SEQ ID NO: 565), |
| | Z36249_PEA_3_T5 (SEQ ID NO: 566) and |
| | Z36249_PEA_3_T9 (SEQ ID NO: 567) |
| Z36249_PEA_3_node_5 (SEQ ID NO: 570) | Z36249_PEA_3_T2 (SEQ ID NO: 564), |
| | Z36249_PEA_3_T3 (SEQ ID NO: 565), |
| | Z36249_PEA_3_T5 (SEQ ID NO: 566) and |
| | Z36249_PEA_3_T9 (SEQ ID NO: 567) |
| Z36249_PEA_3_node_11 (SEQ ID NO: 571) | Z36249_PEA_3_T5 (SEQ ID NO: 566) |
| Z36249_PEA_3_node_14 (SEQ ID NO: 572) | Z36249_PEA_3_T3 (SEQ ID NO: 565) |
| Z36249_PEA_3_node_24 (SEQ ID NO: 573) | Z36249_PEA_3_T2 (SEQ ID NO: 564) and |
| | Z36249_PEA_3_T9 (SEQ ID NO: 567) |
| Z36249_PEA_3_node_10 (SEQ ID NO: 574) | Z36249_PEA_3_T3 (SEQ ID NO: 565), |
| | Z36249_PEA_3_T5 (SEQ ID NO: 566) and |
| | Z36249_PEA_3_T9 (SEQ ID NO: 567) |
| Z36249_PEA_3_node_13 (SEQ ID NO: 575) | Z36249_PEA_3_T2 (SEQ ID NO: 564) and |
| | Z36249_PEA_3_T3 (SEQ ID NO: 565) |
| Z36249_PEA_3_node_17 (SEQ ID NO: 576) | Z36249_PEA_3_T2 (SEQ ID NO: 564) and |
| | Z36249_PEA_3_T9 (SEQ ID NO: 567) |
| Z36249_PEA_3_node_19 (SEQ ID NO: 577) | Z36249_PEA_3_T2 (SEQ ID NO: 564) and |
| | Z36249_PEA_3_T9 (SEQ ID NO: 567) |
| Z36249_PEA_3_node_21 (SEQ ID NO: 578) | Z36249_PEA_3_T2 (SEQ ID NO: 564) and |
| | Z36249_PEA_3_T9 (SEQ ID NO: 567) |

TABLE 231
Proteins of interest

| Protein Name | Corresponding Transcript(s) |
|---|---|
| Z36249_PEA_3_P2 | Z36249_PEA_3_T2 (SEQ ID NO: 564) |
| (SEQ ID NO: 579) | |
| Z36249_PEA_3_P3 | Z36249_PEA_3_T3 (SEQ ID NO: 565) |
| (SEQ ID NO: 580) | |
| Z36249_PEA_3_P4 | Z36249_PEA_3_T5 (SEQ ID NO: 566) |
| (SEQ ID NO: 581) | |
| Z36249_PEA_3_P5 | Z36249_PEA_3_T9 (SEQ ID NO: 567) |
| (SEQ ID NO: 582) | |

These sequences are variants of the known protein ANKRD1 (ankyrin repeat domain 1 (cardiac muscle)) (SwissProt accession identifier Q96LE7_HUMAN (SEQ ID NO:693); known also according to the synonyms Q96LE7), referred to herein as the previously known protein.

According to optional but preferred embodiments of the present invention, variants of this cluster according to the present invention (amino acid and/or nucleic acid sequences of Z36249) may optionally have one or more of the following utilities, as described with regard to Table 232 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 232
Utilities for Variants of Z36249, related to ANKRD1 ankyrin repeat
domain 1 (cardiac muscle):

| Utility | Reason | Reference |
|---|---|---|
| Conductivity problems in the | ANKRD1 plays a | J Mol Cell Cardiol. 2005 |
| heart | pathophysiological role in the | Feb; 38(2): 353-65. Epub 2005 Jan 26 |
| | contractile responsiveness of | |
| | myocardium. | |
| Marker for wound healing | CARP treatment also induced | Am J Pathol. 2005 Jan; 166(1): 303-12; |
| and neuvascularization (in | neovascularization and | Cardiovasc Res. 2003 Sep |
| wound and heart) | increased blood perfusion in | 1; 59(3): 573-81. |
| | rabbit excisional wounds in | |
| | and ischemic rat wounds. | |
| Diagnosis of spinal muscular | CARP was expressed | Pathol Int. 2003 Oct; 53(10): 653-8; |
| atrophy (SMA) congenital | selectively in severely | Lab Invest. 2003 May; 83(5): 711-9. |
| myopathy, and muscular | atrophic myofibers, | |
| dystrophy | suggesting that CARP | |
| | expression may reflect the | |
| | status of muscle atrophy. | |
| Detection of hypertrophic | CARP is induced in the adult | Lab Invest. 2003 May; 83(5): 711-9. |
| heart (with pressure overload) | hypertrophic heart subjected | |
| | to pressure overload | |
| Diagnosis heart failure | CARP mRNA and protein | Biochem Biophys Res Commun. |
| (especially left ventricle), | levels were markedly | 2002 May 24; 293(5): 1377-82; |
| | increased in failing left | Hypertension. 2000 Jul; 36(1): 48-53. |
| | ventricles (obtained from | |
| | heart explants of end stage | |
| | failure hearts) | |

According to other optional embodiments of the present invention, variants or this cluster according to the present invention (amino acid and/or nucleic acid sequences of Z36249) may optionally have one or more of the following utilities, some of which are related to utilities described above. It should be noted that these utilities are optionally and preferably suitable for human and non-human animals as subjects, except where otherwise noted.

Table 233 below describes diagnostic utilities for the cluster Z36249 that were found through microarrays, including the statistical significance thereof and a reference. One or more Z36249 variants according to the present invention may optionally have one or more of these utilities.

TABLE 233

| Utility | Significance | Reference |
|---|---|---|
| Detection of estrogen receptor | OX in 10 out of 10 ERb | GDS884 |
| beta expressing cells for | transfected cells vs 0 out | |
| hormone therapy in breast | of 10 ERa transfected | |
| cancer. | cells. | |
| Detection of classical Glioma. | 9.2E−9 | Cancer Res. |
| Gene OX in classical Glioma | | 2003 Apr 1; |
| Vs. non classical. | | 63(7): 1602-7. |

Other non-limiting exemplary utilities for Z36249 variants according to the present invention are described in greater detail below and also with regard to the previous section on clinical utility.

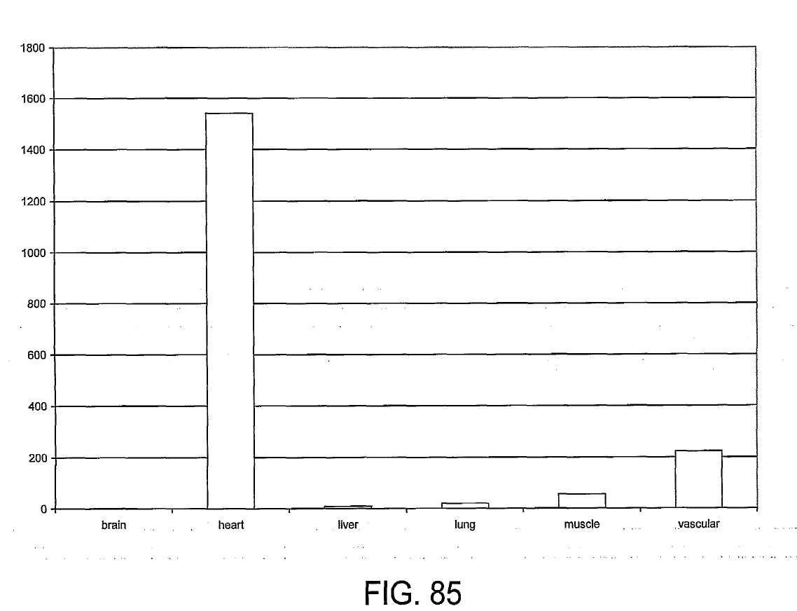

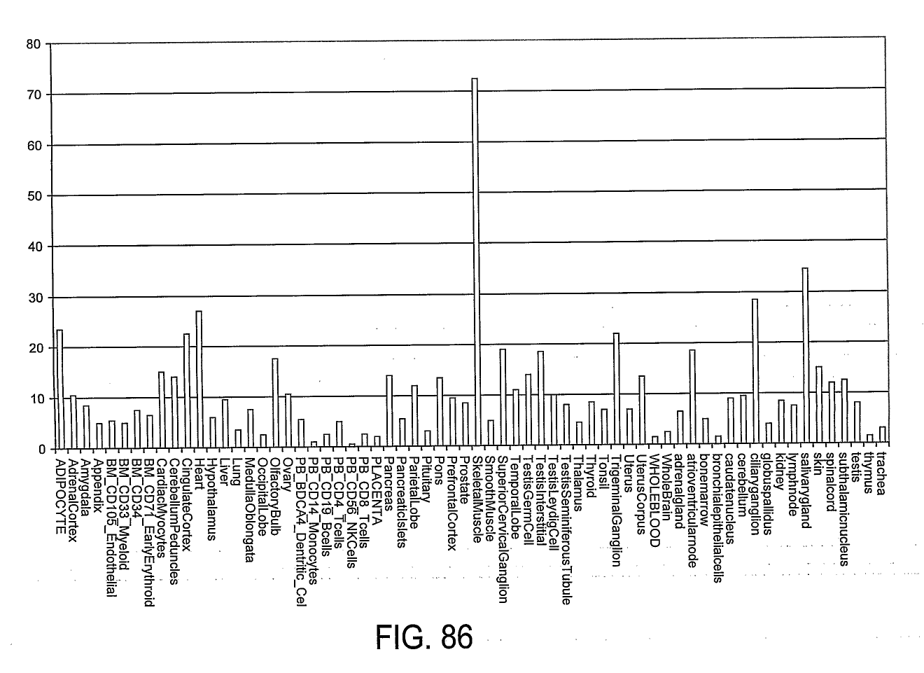

The heart-selective diagnostic marker prediction engine provided the following results with regard to cluster Z36249. Predictions were made for selective expression of transcripts of this cluster in heart tissue, according to the previously described methods. The numbers on the y-axis of FIG. 85 refer to weighted expression of ESTs in each category, as “parts per million” (ratio of the expression of ESTs for a particular cluster to the expression of all ESTs in that category, according to parts per million).

Overall, the following results were obtained as shown with regard to the histogram in FIG. 85, concerning the number of heart-specific clones in libraries/sequences; as well as with regard to the histogram in FIG. 86, concerning the actual expression of oligonucleotides in various tissues, including heart.

This cluster was found to be selectively expressed in heart for the following reasons: in a comparison of the ratio of expression of the cluster in heart specific ESTs to the overall expression of the cluster in non-heart ESTs, which was found to be 33.8; the ratio of expression of the cluster in heart specific ESTs to the overall expression of the cluster in muscle-specific ESTs which was found to be 27.8; and fisher exact test P-values were computed both for library and weighted clone counts to check that the counts are statistically significant, and were found to be 1.60E-47.

One particularly important measure of specificity of expression of a cluster in heart tissue is the previously described comparison of the ratio of expression of the cluster in heart as opposed to muscle. This cluster was found to be specifically expressed in heart as opposed to non-heart ESTs as described above. However, many proteins have been shown to be generally expressed at a higher level in both heart and muscle, which is less desirable. For this cluster, as described above, the ratio of expression of the cluster in heart specific ESTs to the overall expression of the cluster in muscle-specific ESTs which was found to be 33.8, which clearly supports specific expression in heart tissue

As noted above, cluster Z36249 features 4 transcript(s), which were listed in Table 229 above. A description of each variant protein according to the present invention is now provided.

Variant protein Z36249_PEA_{—}3_P2 (SEQ ID NO:579) according to the present invention has an amino acid sequence; it is encoded by transcript(s) Z36249_PEA_{—}3_T2 (SEQ ID NO:564). One or more alignments to one or more previously published protein sequences are given in the alignment table located on the attached CDROM. A brief description of the relationship of the variant protein according to the present invention to each such aligned protein is as follows:

Comparison Report Between Z36249_PEA_{—}3_P2 (SEQ ID NO:579) and Q96LE7:

1. An isolated chimeric polypeptide encoding for Z36249_PEA_{—}3_P2 (SEQ ID NO:579), comprising a first amino acid sequence being at least 90% homologous to MMVLKVEELVTGKKNGNGEAGEFLPEDFRDGEYEAAVTLEKQEDLKTLLAHPVTLGEQQWKSE KQREAELKKKKLEQRSKLENLEDLEIIIQLKKRKKYRKTKVPVVKEPEPEII corresponding to amino acids 1-115 of Q96LE7, which also corresponds to amino acids 1-115 of Z36249_PEA_{—}3_P2 (SEQ ID NO:579), and a second amino acid sequence being at least 90% homologous to YKRTALHRACLEGHLAIVEKLMEAGAQIEFRDMLESTAIHWASRGGNLDVLKLLLNKGAKISARD KLLSTALHVAVRTGHYECAEHLIACEADLNAKDREGDTPLHDAVRLNRYKMIRLLIMYGADLNIK NCAGKTPMDLVLHWQNGTKAIFDSLRENSYKTSRIATF corresponding to amino acids 152-319 of Q96LE7, which also corresponds to amino acids 116-283 of Z36249_PEA_{—}3_P2 (SEQ ID NO:579), wherein said first amino acid sequence and second amino acid sequence are contiguous and in a sequential order.

2. An isolated chimeric polypeptide encoding for an edge portion of Z36249_PEA_{—}3_P2 (SEQ ID NO:579), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IY, having a structure as follows: a sequence starting from any of amino acid numbers 115−x to 115; and ending at any of amino acid numbers 116+((n−2)−x), in which x varies from 0 to n−2.

Comparison Report Between Z36249_PEA_{—}3_P2 (SEQ ID NO:579) and Q15327:

1. An isolated chimeric polypeptide encoding for Z36249_PEA_{—}3_P2 (SEQ ID NO:579), comprising a first amino acid sequence being at least 90% homologous to MMVLKVEELVTGKKNGNGEAGEFLPEDFRDGEYEAAVTLEKQEDLKTLLAHPVTLGEQQWKSE KQREAEL corresponding to amino acids 1-70 of Q15327, which also corresponds to amino acids 1-70 of Z36249_PEA_{—}3_P2 (SEQ ID NO:579), a bridging amino acid K corresponding to amino acid 71 of Z36249_PEA_{—}3_P2 (SEQ ID NO:579), a second amino acid sequence being at least 90% homologous to KKKLEQRSKLENLEDLEIIIQLKKRKKYRKTKVPVVKEPEPEII corresponding to amino acids 72-115 of Q15327, which also corresponds to amino acids 72-115 of Z36249_PEA_{—}3_P2 (SEQ ID NO:579), and a third amino acid sequence being at least 90% homologous to YKRTALHRACLEGHLAIVEKLMEAGAQIEFRDMLESTAIHWASRGGNLDVLKLLLNKGAKISARD KLLSTALHVAVRTGHYECAEHLIACEADLNAKDREGDTPLHDAVRLNRYKMIRLLIMYGADLNIK NCAGKTPMDLVLHWQNGTKAIFDSLRENSYKTSRIATF corresponding to amino acids 152-319 of Q15327, which also corresponds to amino acids 116-283 of Z36249_PEA_{—}3_P2 (SEQ ID NO:579), wherein said first amino acid sequence, bridging amino acid, second amino acid sequence and third amino acid sequence are contiguous and in a sequential order.

2. An isolated chimeric polypeptide encoding for an edge portion of Z36249_PEA_{—}3_P2 (SEQ ID NO:579), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IY, having a structure as follows: a sequence starting from any of amino acid numbers 115−x to 115; and ending at any of amino acid numbers 116+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein Z36249_PEA_{—}3_P2 (SEQ ID NO:579) also has the following non-silent SNPs (Single Nucleotide Polymorphisms) as listed in Table 234, (given according to their position(s) on the amino acid sequence, with the alternative amino acid(s) listed; the last column indicates whether the SNP is known or not; the presence of known SNPs in variant protein Z36249_PEA_{—}3_P2 (SEQ ID NO:579) sequence provides support for the deduced sequence of this variant protein according to the present invention).

TABLE 234
Amino acid mutations

| SNP position(s) on amino | Alternative | Previously |
|---|---|---|
| acid sequence | amino acid(s) | known SNP? |
| 34 | E -> * | Yes |

Variant protein Z36249_PEA_{—}3_P2 (SEQ ID NO:579) is encoded by the following transcript(s): Z36249_PEA_{—}3_T2 (SEQ ID NO:564). The coding portion of transcript Z36249_PEA_{—}3_T2 (SEQ ID NO:564) starts at position 250 and ends at position 1098. The transcript also has the following SNPs as listed in Table 235 (given according to their position on the nucleotide sequence, with the alternative nucleic acid listed; the last column indicates whether the SNP is known or not; the presence of known SNPs in variant protein Z36249_PEA_{—}3_P2 (SEQ ID NO:579) sequence provides support for the deduced sequence of this variant protein according to the present invention).

TABLE 235
Nucleic acid SNPs

| SNP position on nucleotide | Alternative | Previously |
|---|---|---|
| sequence | nucleic acid | known SNP? |
| 105 | T -> C | Yes |
| 208 | T -> | No |
| 349 | G -> T | Yes |
| 459 | C -> A | No |
| 1160 | A -> G | Yes |
| 1356 | C -> T | Yes |
| 1417 | C -> T | Yes |
| 1516 | C -> T | Yes |
| 1601 | C -> T | Yes |
| 1705 | G -> A | Yes |
| 1761 | G -> A | Yes |
| 1969 | G -> A | Yes |
| 1974 | G -> A | Yes |
| 2047 | G -> A | Yes |

Variant protein Z36249_PEA_{—}3_P3 (SEQ ID NO:580) according to the present invention has an amino acid sequence; it is encoded by transcript(s) Z36249_PEA_{—}3_T3 (SEQ ID NO:565). One or more alignments to one or more previously published protein sequences are in the alignment table located on the attached CDROM. A brief description of the relationship of the variant protein according to the present invention to each such aligned protein is as follows:

Comparison Report Between Z36249_PEA_{—}3_P3 (SEQ ID NO:580) and Q96LE7:

1. An isolated chimeric polypeptide encoding for Z36249_PEA_{—}3_P3 (SEQ ID NO:580), comprising a first amino acid sequence being at least 90% homologous to MMVLKVEELVTGKKNGNGEAGEFLPEDFRDGEYEAAVTLEKQEDLKTLLAHPVTLGEQQWKSE KQREAELKKKKLEQRSKLENLEDLEIIIQLKKRKKYRKTKVPVVKEPEPEIITEPVDVPTFLKAALE NKLPVVEKFLSDKNNPDVCDEYKRTALHRACLEGHLAIVEKLMEAGAQIEFRDM corresponding to amino acids 1-184 of Q96LE7, which also corresponds to amino acids 1-184 of Z36249_PEA_{—}3_P3 (SEQ ID NO:580), and a second amino acid sequence being at least 70%, optionally at least 80%, preferably at least 85%, more preferably at least 90% and most preferably at least 95% homologous to a polypeptide having the sequence VNIFLCLGMSQKK (SEQ ID NO: 665) corresponding to amino acids 185-197 of Z36249_PEA_{—}3_P3 (SEQ ID NO:580), wherein said first amino acid sequence and second amino acid sequence are contiguous and in a sequential order.

2. An isolated polypeptide encoding for a tail of Z36249_PEA_{—}3_P3 (SEQ ID NO:580), comprising a polypeptide being at least 70%, optionally at least about 80%, preferably at least about 85%, more preferably at least about 90% and most preferably at least about 95% homologous to the sequence VNIFLCLGMSQKK (SEQ ID NO: 665) in Z36249_PEA_{—}3_P3 (SEQ ID NO:580).

Comparison Report Between Z36249_PEA_{—}3_P3 (SEQ ID NO:580) and Q15327:

1. An isolated chimeric polypeptide encoding for Z36249_PEA_{—}3_P3 (SEQ ID NO:580), comprising a first amino acid sequence being at least 90% homologous to MMVLKVEELVTGKKNGNGEAGEFLPEDFRDGEYEAAVTLEKQEDLKTLLAHPVTLGEQQWKSE KQREAEL corresponding to amino acids 1-70 of Q15327, which also corresponds to amino acids 1-70 of Z36249_PEA_{—}3_P3 (SEQ ID NO:580), a bridging amino acid K corresponding to amino acid 71 of Z36249_PEA_{—}3_P3 (SEQ ID NO:580), a second amino acid sequence being at least 90% homologous to KKKLEQRSKLENLEDLEIIIQLKKRKKYRKTKVPVVKEPEPEIITEPVDVPTFLKAALENKLPVVEKF LSDKNNPDVCDEYKRTALHRACLEGHLAIVEKLMEAGAQIEFRDM corresponding to amino acids 72-184 of Q15327, which also corresponds to amino acids 72-184 of Z36249_PEA_{—}3_P3 (SEQ ID NO:580), and a third amino acid sequence being at least 70%, optionally at least 80%, preferably at least 85%, more preferably at least 90% and most preferably at least 95% homologous to a polypeptide having the sequence VNIFLCLGMSQKK (SEQ ID NO: 665) corresponding to amino acids 185-197 of Z36249_PEA_{—}3_P3 (SEQ ID NO:580), wherein said first amino acid sequence, bridging amino acid, second amino acid sequence and third amino acid sequence are contiguous and in a sequential order.

2. An isolated polypeptide encoding for a tail of Z36249_PEA_{—}3_P3 (SEQ ID NO:580), comprising a polypeptide being at least 70%, optionally at least about 80%, preferably at least about 85%, more preferably at least about 90% and most preferably at least about 95% homologous to the sequence VNIFLCLGMSQKK (SEQ ID NO: 665) in Z36249_PEA_{—}3_P3 (SEQ ID NO:580). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein Z36249_PEA_{—}3_P3 (SEQ ID NO:580) also has the following non-silent SNPs (Single Nucleotide Polymorphisms) as listed in Table 236, (given according to their position(s) on the amino acid sequence, with the alternative amino acid(s) listed; the last column indicates whether the SNP is known or not; the presence of known SNPs in variant protein Z36249_PEA_{—}3_P3 (SEQ ID NO:580) sequence provides support for the deduced sequence of this variant protein according to the present invention).

TABLE 236
Amino acid mutations

| SNP position(s) on amino acid | Alternative | Previously |
|---|---|---|
| sequence | amino acid(s) | known SNP? |
| 34 | E -> * | Yes |

Variant protein Z36249_PEA_{—}3_P3 (SEQ ID NO:580) is encoded by the following transcript(s): Z36249_PEA_{—}3_T3 (SEQ ID NO:565). The coding portion of transcript Z36249 PEA_{—}3_T3 (SEQ ID NO:565) starts at position 250 and ends at position 840. The transcript also has the following SNPs as listed in Table 237 (given according to their position on the nucleotide sequence, with the alternative nucleic acid listed; the last column indicates whether the SNP is known or not; the presence of known SNPs in variant protein Z36249_PEA_{—}3_P3 (SEQ ID NO:580) sequence provides support for the deduced sequence of this variant protein according to the present invention).

TABLE 237
Nucleic acid SNPs

| SNP position on nucleotide | Alternative | Previously |
|---|---|---|
| sequence | nucleic acid | known SNP? |
| 105 | T -> C | Yes |
| 208 | T -> | No |
| 349 | G -> T | Yes |
| 459 | C -> A | No |

Variant protein Z36249_PEA_{—}3_P4 (SEQ ID NO:581) according to the present invention has an amino acid sequence; it is encoded by transcript(s) Z36249_PEA_{—}3_T5 (SEQ ID NO:566). One or more alignments to one or more previously published protein sequences are in the alignment table located on the attached CDROM. A brief description of the relationship of the variant protein according to the present invention to each such aligned protein is as follows:

Comparison Report Between Z36249_PEA_{—}3_P4 (SEQ ID NO:581) and Q96LE7:

1. An isolated chimeric polypeptide encoding for Z36249_PEA_{—}3_P4 (SEQ ID NO:581), comprising a first amino acid sequence being at least 90% homologous to MMVLKVEELVTGKKNGNGEAGEFLPEDFRDGEYEAAVTLEKQEDLKTLLAHPVTLGEQQWKSE KQREAELKKKKLEQRSKLENLEDLEIIIQLKKRKKYRKTKVPVVKEPEPEIITEPVDVPTFLKAALE NKLPVVEKFLSDKNNPDVCDE corresponding to amino acids 1-151 of Q96LE7, which also corresponds to amino acids 1-151 of Z36249_PEA_{—}3_P4 (SEQ ID NO:581), and a second amino acid sequence being at least 70%, optionally at least 80%, preferably at least 85%, more preferably at least 90% and most preferably at least 95% homologous to a polypeptide having the sequence VRLMQSTAKSSSLILCFLCFTPVLLI (SEQ ID NO: 666) corresponding to amino acids 152-177 of Z36249_PEA_{—}3_P4 (SEQ ID NO:581), wherein said first amino acid sequence and second amino acid sequence are contiguous and in a sequential order.

2. An isolated polypeptide encoding for a tail of Z36249_PEA_{—}3_P4 (SEQ ID NO:581), comprising a polypeptide being at least 70%, optionally at least about 80%, preferably at least about 85%, more preferably at least about 90% and most preferably at least about 95% homologous to the sequence VRLMQSTAKSSSLILCFLCFTPVLLI (SEQ ID NO: 666) in Z36249_PEA_{—}3_P4 (SEQ ID NO:581).

Comparison Report Between Z36249_PEA_{—}3_P4 (SEQ ID NO:581) and Q15327:

1. An isolated chimeric polypeptide encoding for Z36249_PEA_{—}3_P4 (SEQ ID NO:581), comprising a first amino acid sequence being at least 90% homologous to MMVLKVEELVTGKKNGNGEAGEFLPEDFRDGEYEAAVTLEKQEDLKTLLAHPVTLGEQQWKSE KQREAEL corresponding to amino acids 1-70 of Q15327, which also corresponds to amino acids 1-70 of Z36249_PEA_{—}3_P4 (SEQ ID NO:581), a bridging amino acid K corresponding to amino acid 71 of Z36249_PEA_{—}3_P4 (SEQ ID NO:581), a second amino acid sequence being at least 90% homologous to KKKLEQRSKLENLEDLEIIIQLKKRKKYRKTKVPVVKEPEPEIITEPVDVPTFLKAALENKLPVVEKF LSDKNNPDVCDE corresponding to amino acids 72-151 of Q15327, which also corresponds to amino acids 72-151 of Z36249_PEA_{—}3_P4 (SEQ ID NO:581), and a third amino acid sequence being at least 70%, optionally at least 80%, preferably at least 85%, more preferably at least 90% and most preferably at least 95% homologous to a polypeptide having the sequence VRLMQSTAKSSSLILCFLCFTPVLLI (SEQ ID NO: 666) corresponding to amino acids 152-177 of Z36249_PEA_{—}3_P4 (SEQ ID NO:581), wherein said first amino acid sequence, bridging amino acid, second amino acid sequence and third amino acid sequence are contiguous and in a sequential order.

2. An isolated polypeptide encoding for a tail of Z36249_PEA_{—}3_P4 (SEQ ID NO:581), comprising a polypeptide being at least 70%, optionally at least about 80%, preferably at least about 85%, more preferably at least about 90% and most preferably at least about 95% homologous to the sequence VRLMQSTAKSSSLILCFLCFTPVLLI (SEQ ID NO: 666) in Z36249_PEA_{—}3_P4 (SEQ ID NO:581).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because only one of the two trans-membrane region prediction programs (Tmpred: 1, Tmhmm: 0) has predicted that this protein has a trans-membrane region. In addition both signal-peptide prediction programs predict that this protein is a non-secreted protein.

Variant protein Z36249_PEA_{—}3_P4 (SEQ ID NO:581) also has the following non-silent SNPs (Single Nucleotide Polymorphisms) as listed in Table 238, (given according to their position(s) on the amino acid sequence, with the alternative amino acid(s) listed; the last column indicates whether the SNP is known or not; the presence of known SNPs in variant protein Z36249_PEA_{—}3_P4 (SEQ ID NO:581) sequence provides support for the deduced sequence of this variant protein according to the present invention).

TABLE 238
Amino acid mutations

| SNP position(s) on amino acid | Alternative | Previously |
|---|---|---|
| sequence | amino acid(s) | known SNP? |
| 34 | E -> * | Yes |

Variant protein Z36249_PEA_{—}3_P4 (SEQ ID NO:581) is encoded by the following transcript(s): Z36249_PEA_{—}3_T5 (SEQ ID NO:566). The coding portion of transcript Z36249_PEA_{—}3_T5 (SEQ ID NO:566) starts at position 250 and ends at position 780. The transcript also has the following SNPs as listed in Table 239 (given according to their position on the nucleotide sequence, with the alternative nucleic acid listed; the last column indicates whether the SNP is known or not; the presence of known SNPs in variant protein Z36249_PEA_{—}3_P4 (SEQ ID NO:581) sequence provides support for the deduced sequence of this variant protein according to the present invention).

TABLE 239
Nucleic acid SNPs

| SNP position on nucleotide | Alternative | Previously |
|---|---|---|
| sequence | nucleic acid | known SNP? |
| 105 | T -> C | Yes |
| 208 | T -> | No |
| 349 | G -> T | Yes |
| 459 | C -> A | No |
| 1265 | T -> C | Yes |

Variant protein Z36249_PEA_{—}3_P5 (SEQ ID NO:582) according to the present invention has an amino acid sequence; it is encoded by transcript(s) Z36249_PEA_{—}3_T9 (SEQ ID NO:567). One or more alignments to one or more previously published protein sequences are in the alignment table located on the attached CDROM. A brief description of the relationship of the variant protein according to the present invention to each such aligned protein is as follows:

Comparison Report Between Z36249_PEA_{—}3_P5 (SEQ ID NO:582) and Q96LE7:

1. An isolated chimeric polypeptide encoding for Z36249_PEA_{—}3_P5 (SEQ ID NO:582), comprising a first amino acid sequence being at least 90% homologous to MMVLKVEELVTGKKNGNGEAGEFLPEDFRDGEYEAAVTLEKQEDLKTLLAHPVTLGEQQWKSE KQREAELKKKKLEQRSKLENLEDLEIIIQLKKRKKYRKTKVPVVKEPEPEIITEPVDVPTFLKAALE NKLPVVEKFLSDKNNPDVCDE corresponding to amino acids 1-151 of Q96LE7, which also corresponds to amino acids 1-151 of Z36249_PEA_{—}3_P5 (SEQ ID NO:582), and a second amino acid sequence being at least 90% homologous to LESTAIHWASRGGNLDVLKLLLNKGAKISARDKLLSTALHVAVRTGHYECAEHLIACEADLNAKD REGDTPLHDAVRLNRYKMIRLLIMYGADLNIKNCAGKTPMDLVLHWQNGTKAIFDSLRENSYKTS RIATF corresponding to amino acids 185-319 of Q96LE7, which also corresponds to amino acids 152-286 of Z36249_PEA_{—}3_P5 (SEQ ID NO:582), wherein said first amino acid sequence and second amino acid sequence are contiguous and in a sequential order.

2. An isolated chimeric polypeptide encoding for an edge portion of Z36249_PEA_{—}3_P5 (SEQ ID NO:582), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EL, having a structure as follows: a sequence starting from any of amino acid numbers 151−x to 151; and ending at any of amino acid numbers 152+((n−2)−x), in which x varies from 0 to n−2.

Comparison Report Between Z36249_PEA_{—}3_P5 (SEQ ID NO:582) and Q15327:

1. An isolated chimeric polypeptide encoding for Z36249_PEA_{—}3_P5 (SEQ ID NO:582), comprising a first amino acid sequence being at least 90% homologous to MMVLKVEELVTGKKNGNGEAGEFLPEDFRDGEYEAAVTLEKQEDLKTLLAHPVTLGEQQWKSE KQREAEL corresponding to amino acids 1-70 of Q15327, which also corresponds to amino acids 1-70 of Z36249_PEA_{—}3_P5 (SEQ ID NO:582), a bridging amino acid K corresponding to amino acid 71 of Z36249_PEA_{—}3_P5 (SEQ ID NO:582), a second amino acid sequence being at least 90% homologous to KKKLEQRSKLENLEDLEIIIQLKKRKKYRKTKVPVVKEPEPEIITEPVDVPTFLKAALENKLPVVEKF LSDKNNPDVCDE corresponding to amino acids 72-151 of Q15327, which also corresponds to amino acids 72-151 of Z36249_PEA_{—}3_P5 (SEQ ID NO:582), and a third amino acid sequence being at least 90% homologous to LESTAIHWASRGGNLDVLKLLLNKGAKISARDKLLSTALHVAVRTGHYECAEHLIACEADLNAKD REGDTPLHDAVRLNRYKMIRLLIMYGADLNIKNCAGKTPMDLVLHWQNGTKAIFDSLRENSYKTS RIATF corresponding to amino acids 185-319 of Q15327, which also corresponds to amino acids 152-286 of Z36249_PEA_{—}3_P5 (SEQ ID NO:582), wherein said first amino acid sequence, bridging amino acid, second amino acid sequence and third amino acid sequence are contiguous and in a sequential order.

2. An isolated chimeric polypeptide encoding for an edge portion of Z36249_PEA_{—}3_P5 (SEQ ID NO:582), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EL, having a structure as follows: a sequence starting from any of amino acid numbers 151−x to 151; and ending at any of amino acid numbers 152+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein Z36249_PEA_{—}3_P5 (SEQ ID NO:582) also has the following non-silent SNPs (Single Nucleotide Polymorphisms) as listed in Table 240, (given according to their position(s) on the amino acid sequence, with the alternative amino acid(s) listed; the last column indicates whether the SNP is known or not; the presence of known SNPs in variant protein Z36249_PEA_{—}3_P5 (SEQ ID NO:582) sequence provides support for the deduced sequence of this variant protein according to the present invention).

TABLE 240
Amino acid mutations

| SNP position(s) on amino acid | Alternative | Previously |
|---|---|---|
| sequence | amino acid(s) | known SNP? |
| 34 | E -> * | Yes |

Variant protein Z36249_PEA_{—}3_P5 (SEQ ID NO:582) is encoded by the following transcript(s): Z36249_PEA_{—}3_T9 (SEQ ID NO:567). The coding portion of transcript Z36249_PEA_{—}3_T9 (SEQ ID NO:567) starts at position 250 and ends at position 1107. The transcript also has the following SNPs as listed in Table 241 (given according to their position on the nucleotide sequence, with the alternative nucleic acid listed; the last column indicates whether the SNP is known or not; the presence of known SNPs in variant protein Z36249_PEA_{—}3_P5 (SEQ ID NO:582) sequence provides support for the deduced sequence of this variant protein according to the present invention).

TABLE 241
Nucleic acid SNPs

| SNP position on nucleotide | Alternative | Previously |
|---|---|---|
| sequence | nucleic acid | known SNP? |
| 105 | T -> C | Yes |
| 208 | T -> | No |
| 349 | G -> T | Yes |
| 459 | C -> A | No |
| 1169 | A -> G | Yes |
| 1365 | C -> T | Yes |
| 1426 | C -> T | Yes |
| 1525 | C -> T | Yes |
| 1610 | C -> T | Yes |
| 1714 | G -> A | Yes |
| 1770 | G -> A | Yes |

Table 242 below describes the starting and ending position of Z36249_PEA_{—}3_node_{—}11 (SEQ ID NO:571) on the relevant transcript. Experimental results for this segment are described below.

TABLE 242
Segment location on transcripts

| | Segment | Segment |
|---|---|---|
| Transcript name | starting position | ending position |
| Z36249_PEA_3_T5 | 703 | 1387 |
| (SEQ ID NO: 566) | | |

Table 243 below describes the starting and ending position of Z36249_PEA_{—}3_node_{—}14 (SEQ ID NO:572) on the relevant transcript. Experimental results for this segment are described below.

TABLE 243
Segment location on transcripts

| | Segment | Segment |
|---|---|---|
| Transcript name | starting position | ending position |
| Z36249_PEA_3_T3 | 802 | 1472 |
| (SEQ ID NO: 565) | | |

Table 244 below describes the starting and ending position of Z36249_PEA_{—}3_node_{—}10 (SEQ ID NO:574) on each of the relevant transcripts. Experimental results for this segment are described below.

TABLE 244
Segment location on transcripts

| | | Segment |
|---|---|---|
| | Segment | ending |
| Transcript name | starting position | position |
| Z36249_PEA_3_T3 (SEQ ID NO: 565) | 595 | 702 |
| Z36249_PEA_3_T5 (SEQ ID NO: 566) | 595 | 702 |
| Z36249_PEA_3_T9 (SEQ ID NO: 567) | 595 | 702 |

Table 245 below describes the starting and ending position of Z36249_PEA_{—}3_node_{—}13 (SEQ ID NO:575) on each of the relevant transcripts. Experimental results for this segment are described below.

TABLE 245
Segment location on transcripts

| | | Segment |
|---|---|---|
| | Segment | ending |
| Transcript name | starting position | position |
| Z36249_PEA_3_T2 (SEQ ID NO: 564) | 595 | 693 |
| Z36249_PEA_3_T3 (SEQ ID NO: 565) | 703 | 801 |

Expression of Homo sapiens ankyrin repeat domain 1 (cardiac muscle) Z36249 transcripts which are detectable by amplicon as depicted in sequence name Z36249 seg11-12 (SEQ ID NO:585) specifically in heart tissue

Expression of Homo sapiens ankyrin repeat domain 1 (cardiac muscle) transcripts detectable by or according to seg11-12 node, Z36249 seg11-12 (SEQ ID NO:585) amplicon and primers Z36249 seg11-12F (SEQ ID NO:583) and Z36249 seg11-12R (SEQ ID NO:584) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44-46, Table 7, above, “Tissue samples in normal panel”), to obtain a value of relative expression for each sample relative to median of the heart samples.

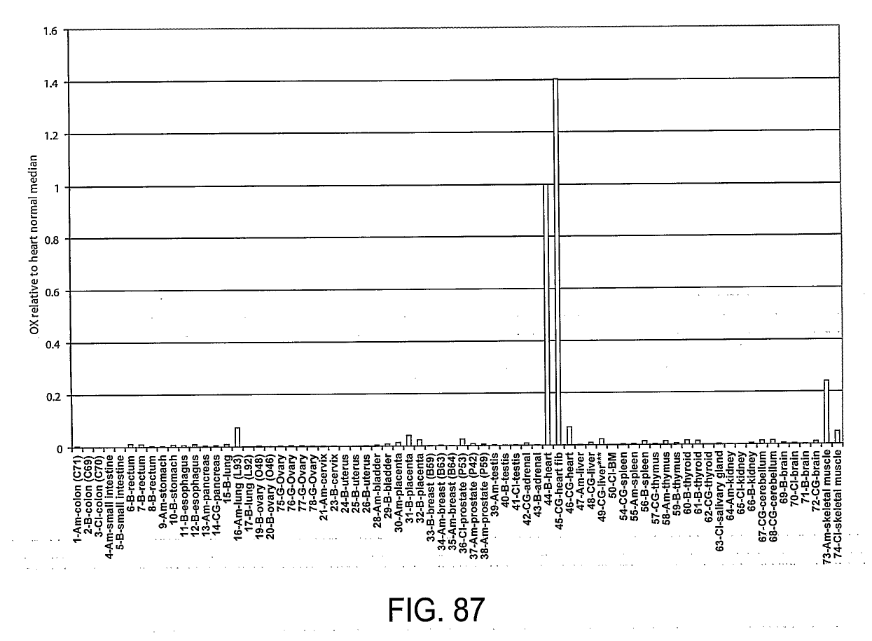

FIG. 87 is a histogram showing relative expression of the above-indicated Homo sapiens ankyrin repeat domain 1 (cardiac muscle) transcripts in heart tissue samples as opposed to other tissues.

As is evident from FIG. 87, the expression of Homo sapiens ankyrin repeat domain 1 (cardiac muscle) transcripts detectable by the above amplicon in heart tissue samples was significantly higher than in the other samples (Sample Nos. 1-43, 47-78 Table 7, “Tissue samples in normal panel).

Primer pairs are also optionally and preferably encompassed within the present invention; for example, for the above experiment, the following primer pair was used as a non-limiting illustrative example only of a suitable primer pair: Z36249 seg11-12F (SEQ ID NO:583) forward primer; and Z36249 seg11-12R (SEQ ID NO:584) reverse primer.

The present invention also preferably encompasses any amplicon obtained through the use of any suitable primer pair; for example, for the above experiment, the following amplicon was obtained as a non-limiting illustrative example only of a suitable amplicon: Z36249 seg11-12 (SEQ ID NO:585).

Primers:

| | |
|---|---|
| Forward primer Z36249 seg11-12F: | (SEQ ID NO: 583) |
| GCTCTGGAGAATAAACTGCCAGTAG | |
| Reverse primer Z36249 seg11-12R: | (SEQ ID NO: 584) |
| TATTAGCAAGACTGGAGTAAAGCATAAG | |
| Amplicon Z36249 seg11-12: | (SEQ ID NO: 585) |

GCTCTGGAGAATAAACTGCCAGTAGTAGAAAAATTCTTGTCAGACAAGAACAATCCAGATGTT
TGTGATGAGGTAAGACTCATGCAAAGCACTGCAAAATCCAGCTCATTAATTTTATGTTTCTTAT
GCTTTACTCCAGTCTTGCTAATA

The table below provides the conversion names for the primers and amplicons used herein.

| | Location in POS3 (Description for |
|---|---|
| Primers/amplicon names | cluster Z36249 section above) |
| Z36249 seg 11-12 (SEQ ID NO: 585) | seg10-11 |

Expression of Homo sapiens ankyrin repeat domain 1 (cardiac muscle) Z36249 transcripts which are detectable by amplicon as depicted in sequence name Z36249 seg14-16 (SEQ ID NO:588) specifically in heart tissue:

Expression of Homo sapiens ankyrin repeat domain 1 (cardiac muscle) transcripts detectable by or according to seg14-16 node, Z36249 seg14-16 (SEQ ID NO:588) amplicon and primers Z36249 seg14-16F (SEQ ID NO:586) and Z36249 seg14-16R (SEQ ID NO:587) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44-46, Table 7, above, “Tissue samples in normal panel), to obtain a value of relative expression for each sample relative to median of the heart samples.

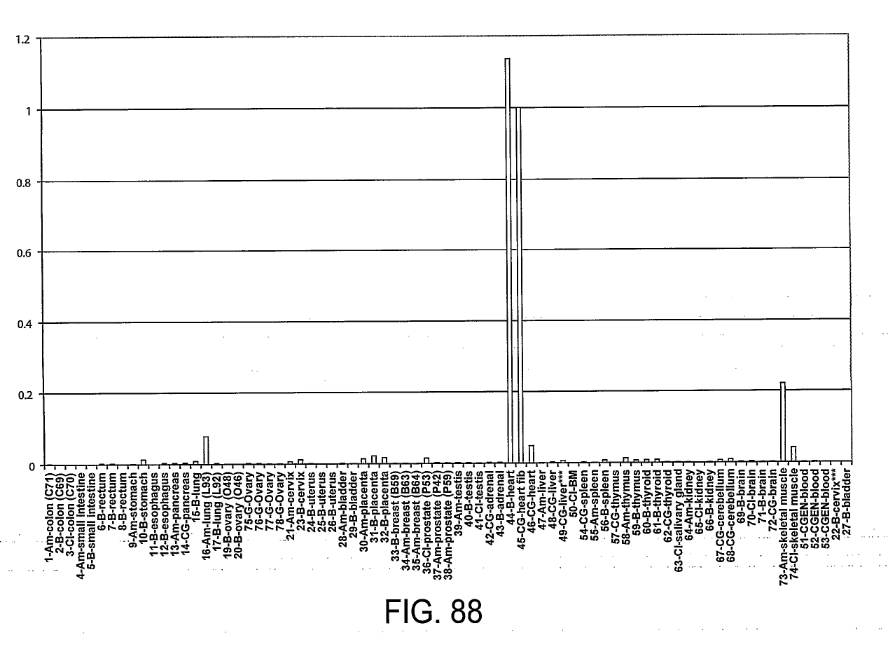

FIG. 88 is a histogram showing relative expression of the above-indicated Homo sapiens ankyrin repeat domain 1 (cardiac muscle) transcripts in heart tissue samples as opposed to other tissues.

As is evident from FIG. 88, the expression of Homo sapiens ankyrin repeat domain 1 (cardiac muscle) transcripts detectable by the above amplicon in heart tissue samples was significantly higher than in the other samples (Sample Nos. 1-43, 47-78 Table 7, “Tissue samples in normal panel).

Primer pairs are also optionally and preferably encompassed within the present invention; for example, for the above experiment, the following primer pair was used as a non-limiting illustrative example only of a suitable primer pair: Z36249 seg14-16F (SEQ ID NO:586) forward primer; and Z36249 seg14-16R (SEQ ID NO:587) reverse primer.

The present invention also preferably encompasses any amplicon obtained through the use of any suitable primer pair; for example, for the above experiment, the following amplicon was obtained as a non-limiting illustrative example only of a suitable amplicon: Z36249 seg14-16 (SEQ ID NO:588).

Primers:

| | |
|---|---|
| Forward primer Z36249 seg14-16F: | (SEQ ID NO: 586) |
| GCTTGGAAGGACATTTGGCA | |
| Reverse primer Z36249 seg14-16R: | (SEQ ID NO: 587) |
| TGGCTCATTCCCAAGCAAAG | |
| Amplicon Z36249 seg14-16: | (SEQ ID NO: 588) |

GCTTGGAAGGACATTTGGCAATTGTGGAGAAGTTAATGGAAGCTGGAGCCCAGATCGAATTC
CGTGATATGGTAAATATATTTCTTTGCTTGGGAATGAGCCA

The table below provides the conversion names for the primers and amplicons used herein.

| | Location in POS3 (Description for |
|---|---|
| Primers/amplicon names | cluster Z36249 section above) |
| Z36249 seg 14-16 (SEQ ID NO: 588) | seg13-14 |

Expression of Homo sapiens ankyrin repeat domain 1 (cardiac muscle) Z36249 transcripts which are detectable by amplicon as depicted in sequence name Z36249 junc23-25 (SEQ ID NO:591) specifically in heart tissue:

Expression of Homo sapiens ankyrin repeat domain 1 (cardiac muscle) transcripts detectable by or according to junc23-25, Z36249 junc23-25 (SEQ ID NO:591) amplicon and primers Z36249 junc23-25F2 (SEQ ID NO:589) and Z36249 junc23-25R2 (SEQ ID NO:590) was measured by real time PCR. In parallel the expression of four housekeeping genes—RPL19 (GenBank Accession No. NM_{—}000981 (SEQ ID NO:7); RPL19 amplicon (SEQ ID NO: 38)), TATA box (GenBank Accession No. NM_{—}003194 (SEQ ID NO:2); TATA amplicon (SEQ ID NO: 53)), Ubiquitin (GenBank Accession No. BC000449 (SEQ ID NO:9); amplicon—Ubiquitin-amplicon (SEQ ID NO:50)) and SDHA (GenBank Accession No. NM_{—}004168 (SEQ ID NO:4); amplicon—SDHA-amplicon (SEQ ID NO:29)), was measured similarly. For each RT sample, the expression of the above amplicons was normalized to the geometric mean of the quantities of the housekeeping genes. The normalized quantity of each RT sample was then divided by the median of the quantities of the heart samples (Sample Nos. 44-46, Table 7, above, “Tissue samples in normal panel), to obtain a value of relative expression for each sample relative to median of the heart samples.

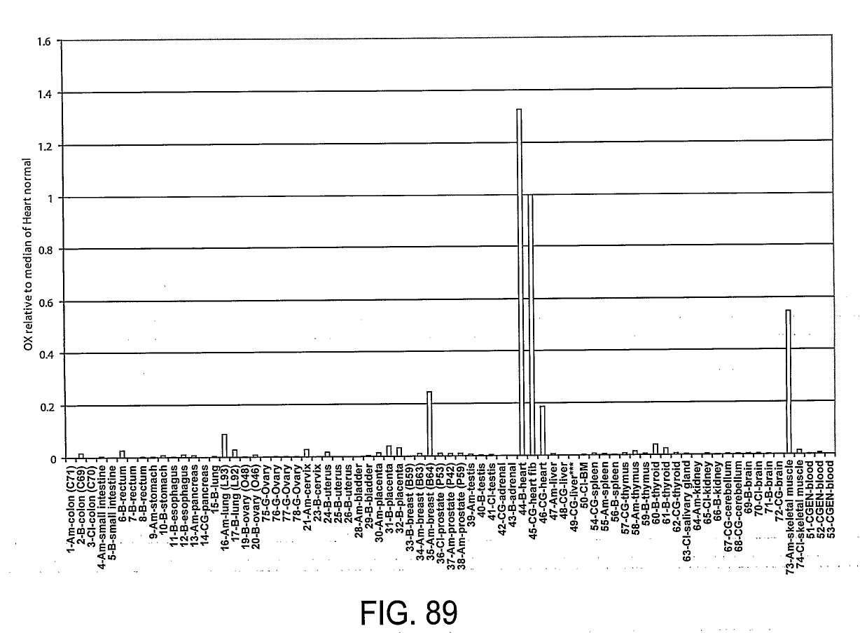

FIG. 89 is a histogram showing relative expression of the above-indicated Homo sapiens ankyrin repeat domain 1 (cardiac muscle) transcripts in heart tissue samples as opposed to other tissues.

As is evident from FIG. 89, the expression of Homo sapiens ankyrin repeat domain 1 (cardiac muscle) transcripts detectable by the above amplicon in heart tissue samples was significantly higher than in the other samples (Sample Nos. 1-43, 47-78, Table 7, “Tissue samples in normal panel).

Primer pairs are also optionally and preferably encompassed within the present invention; for example, for the above experiment, the following primer pair was used as a non-limiting illustrative example only of a suitable primer pair: Z36249 junc23-25F2 (SEQ ID NO:589) forward primer; and Z36249 junc23-25R2 (SEQ ID NO: 590) reverse primer.

The present invention also preferably encompasses any amplicon obtained through the use of any suitable primer pair; for example, for the above experiment, the following amplicon was obtained as a non-limiting illustrative example only of a suitable amplicon: Z36249 junc23-25F2R2 (SEQ ID NO: 591).

Primers:

| | |
|---|---|
| Forward primer Z36249 junc23-25F2- | (SEQ ID NO: 589) |
| CAGCGCTGCATGTGGC | |
| Reverse primer Z36249 junc23-25R2- | (SEQ ID NO: 590) |
| CAACGGGGTATCTCCTTCTCTG | |
| Amplicon Z36249 junc23-25F2R2- | (SEQ ID NO: 591) |

CAGCGCTGCATGTGGCGGTGAGGACTGGCCACTATGAGTGCGCGGAGCATCTTATCGCCTGTG
AGGCAGACCTCAACGCCAAAGACAGAGAAGGAGATACCCCGTTG

The table below provides the conversion names for the primers and amplicons used herein.

| | Location in POS3 (Description for |
|---|---|
| Primers/amplicon names | cluster Z36249 section above) |
| Z36249 junc 23-25F2R2 (SEQ | Junc 19-21 |
| ID NO: 591) | |

### Description for Cluster M78530

Cluster M78530 features 3 transcript(s) and 21 segment(s) of interest, the names for which are given in Tables 246 and 247, respectively. The selected protein variants are given in table 248.

TABLE 246
Transcripts of interest
Transcript Name

M78530_PEA_1_T11 (SEQ ID NO: 592)
M78530_PEA_1_T12 (SEQ ID NO: 593)
M78530_PEA_1_T13 (SEQ ID NO: 594)

TABLE 247
Segments of interest

| Segment Name | |
|---|---|
| M78530_PEA_1_node_0 (SEQ ID NO: 595) | M78530_PEA_1_T11 (SEQ ID NO: 592), |
| | M78530_PEA_1_T12 (SEQ ID NO: 593) and |
| | M78530_PEA_1_T13 (SEQ ID NO: 594) |
| M78530_PEA_1_node_15 (SEQ ID NO: 596) | M78530_PEA_1_T11 (SEQ ID NO: 592), |
| | M78530_PEA_1_T12 (SEQ ID NO: 593) and |
| | M78530_PEA_1_T13 (SEQ ID NO: 594) |
| M78530_PEA_1_node_16 (SEQ ID NO: 597) | M78530_PEA_1_T13 (SEQ ID NO: 594) |
| M78530_PEA_1_node_19 (SEQ ID NO: 598) | M78530_PEA_1_T12 (SEQ ID NO: 593) |
| M78530_PEA_1_node_21 (SEQ ID NO: 599) | M78530_PEA_1_T11 (SEQ ID NO: 592) |
| M78530_PEA_1_node_23 (SEQ ID NO: 600) | M78530_PEA_1_T11 (SEQ ID NO: 592) |
| M78530_PEA_1_node_27 (SEQ ID NO: 601) | M78530_PEA_1_T11 (SEQ ID NO: 592) |
| M78530_PEA_1_node_29 (SEQ ID NO: 602) | M78530_PEA_1_T11 (SEQ ID NO: 592) |
| M78530_PEA_1_node_36 (SEQ ID NO: 603) | M78530_PEA_1_T11 (SEQ ID NO: 592) |
| M78530_PEA_1_node_37 (SEQ ID NO: 604) | M78530_PEA_1_T11 (SEQ ID NO: 592) |
| M78530_PEA_1_node_2 (SEQ ID NO: 605) | M78530_PEA_1_T11 (SEQ ID NO: 592), |
| | M78530_PEA_1_T12 (SEQ ID NO: 593) and |
| | M78530_PEA_1_T13 (SEQ ID NO: 594) |
| M78530_PEA_1_node_4 (SEQ ID NO: 606) | M78530_PEA_1_T11 (SEQ ID NO: 592), |
| | M78530_PEA_1_T12 (SEQ ID NO: 593) and |
| | M78530_PEA_1_T13 (SEQ ID NO: 594) |
| M78530_PEA_1_node_5 (SEQ ID NO: 607) | M78530_PEA_1_T11 (SEQ ID NO: 592), |
| | M78530_PEA_1_T12 (SEQ ID NO: 593) and |
| | M78530_PEA_1_T13 (SEQ ID NO: 594) |
| M78530_PEA_1_node_7 (SEQ ID NO: 608) | M78530_PEA_1_T11 (SEQ ID NO: 592), |
| | M78530_PEA_1_T12 (SEQ ID NO: 593) and |
| | M78530_PEA_1_T13 (SEQ ID NO: 594) |
| M78530_PEA_1_node_9 (SEQ ID NO: 609) | M78530_PEA_1_T11 (SEQ ID NO: 592), |
| | M78530_PEA_1_T12 (SEQ ID NO: 593) and |
| | M78530_PEA_1_T13 (SEQ ID NO: 594) |
| M78530_PEA_1_node_10 (SEQ ID NO: 610) | M78530_PEA_1_T11 (SEQ ID NO: 592), |
| | M78530_PEA_1_T12 (SEQ ID NO: 593) and |
| | M78530_PEA_1_T13 (SEQ ID NO: 594) |
| M78530_PEA_1_node_18 (SEQ ID NO: 611) | M78530_PEA_1_T11 (SEQ ID NO: 592) and |
| | M78530_PEA_1_T12 (SEQ ID NO: 593) |
| M78530_PEA_1_node_25 (SEQ ID NO: 612) | M78530_PEA_1_T11 (SEQ ID NO: 592) |
| M78530_PEA_1_node_30 (SEQ ID NO: 613) | M78530_PEA_1_T11 (SEQ ID NO: 592) |
| M78530_PEA_1_node_33 (SEQ ID NO: 614) | M78530_PEA_1_T11 (SEQ ID NO: 592) |
| M78530_PEA_1_node_34 (SEQ ID NO: 615) | M78530_PEA_1_T11 (SEQ ID NO: 592) |

TABLE 248
Proteins of interest

| Protein Name | Corresponding Transcript(s) |
|---|---|
| M78530_PEA_1_P15 (SEQ ID | M78530_PEA_1_T11 (SEQ ID |
| NO: 619) | NO: 592) |
| M78530_PEA_1_P16 (SEQ ID | M78530_PEA_1_T12 (SEQ ID |
| NO: 620) | NO: 593) |
| M78530_PEA_1_P17 (SEQ ID | M78530_PEA_1_T13 (SEQ ID |
| NO: 621) | NO: 594) |

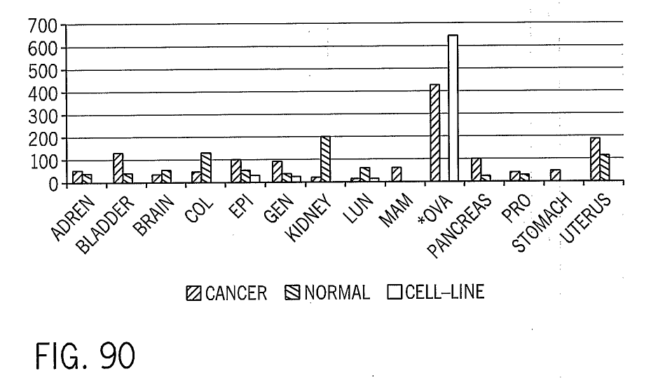

Cluster M78530 can be used as a diagnostic marker according to overexpression of transcripts of this cluster in cancer. Expression of such transcripts in normal tissues is also given according to the previously described methods. The term “number” in the left hand column of the table and the numbers on the y-axis of FIG. 90 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 90 and Table 249. This cluster is overexpressed (at least at a minimum level) in the following pathological conditions: ovarian carcinoma. P values and ratios for expression in cancerous tissue are described in table 250.

TABLE 249
Normal tissue distribution

| Name of Tissue | Number |
|---|---|
| Adrenal | 40 |
| Bladder | 41 |
| Brain | 52 |
| Colon | 126 |
| Epithelial | 51 |
| General | 35 |
| Kidney | 199 |
| Lung | 63 |
| Breast | 0 |
| Ovary | 0 |
| Pancreas | 20 |
| Prostate | 28 |
| Stomach | 0 |
| Uterus | 113 |

TABLE 250
P values and ratios for expression in cancerous tissue

| Name of Tissue | P1 | P2 | SP1 | R3 | SP2 | R4 |
|---|---|---|---|---|---|---|
| adrenal | 6.4e−01 | 6.9e−01 | 7.1e−01 | 1.1 | 7.8e−01 | 0.9 |
| bladder | 3.3e−01 | 4.5e−01 | 2.8e−01 | 2.0 | 4.9e−01 | 1.4 |
| brain | 7.9e−01 | 8.1e−01 | 8.5e−01 | 0.6 | 9.8e−01 | 0.4 |
| colon | 4.7e−01 | 6.1e−01 | 9.7e−01 | 0.5 | 9.9e−01 | 0.4 |
| epithelial | 2.0e−01 | 8.2e−01 | 3.3e−01 | 1.6 | 2.5e−01 | 0.9 |
| general | 1.3e−01 | 8.5e−01 | 7.4e−01 | 2.2 | 6.0e−04 | 1.4 |
| kidney | 7.0e−01 | 7.6e−01 | 1 | 0.2 | 1 | 0.1 |
| lung | 8.6e−01 | 9.1e−01 | 1 | 0.3 | 1 | 0.3 |
| breast | 1.9e−01 | 2.8e−01 | 3.3e−01 | 2.4 | 5.6e−01 | 1.6 |
| ovary | 1.6e−02 | 1.3e−02 | 7.0e−05 | 10.3 | 6.3e−06 | 9.3 |
| pancreas | 2.6e−01 | 4.1e−01 | 3.5e−02 | 2.2 | 1.2e−01 | 1.5 |
| prostate | 7.9e−01 | 8.6e−01 | 4.7e−01 | 1.2 | 6.3e−01 | 1.0 |
| stomach | 1.1e−01 | 4.5e−01 | 5.0e−01 | 2.2 | 8.0e−01 | 1.3 |
| uterus | 5.3e−01 | 8.2e−01 | 2.4e−01 | 1.0 | 7.7e−01 | 0.6 |

For this cluster, at least one oligonucleotide was found to demonstrate overexpression of the cluster,

For this cluster, at least one oligonucleotide was found to demonstrate overexpression of the cluster, although not of at least one transcript/segment as listed below. Microarray (chip) data is also available for this cluster as follows. Various oligonucleotides were tested for being differentially expressed in various disease conditions, particularly cancer, as previously described. The following oligonucleotides were found to hit this cluster but not other segments/transcripts below (in relation to ovarian cancer), shown in Table 251.

TABLE 251
Oligonucleotides related to this cluster

| | Overexpressed | Chip |
|---|---|---|
| Oligonucleotide name | in cancers | reference |
| M78530_0_6_0 (SEQ ID NO: 12) | ovarian carcinoma | OVA |

The sequence for M78530_{—}0_{—}6_{—}0 (SEQ ID NO:12) is as follows: CTTCCTACACACATCTAGACGTTCAAGTTTGCAAATCAGTTTTTAGCAAG.

As noted above, cluster M78530 features 3 transcript(s), which were listed in Table 246 above. A description of each variant protein according to the present invention is now provided.

Variant protein M78530_PEA_{—}1_P15 (SEQ ID NO:619) according to the present invention has an amino acid sequence; it is encoded by transcript(s) M78530_PEA_{—}1_T11 (SEQ ID NO:592). One or more alignments to one or more previously published protein sequences are in the alignment table located on the attached CDROM. A brief description of the relationship of the variant protein according to the present invention to each such aligned protein is as follows:

Comparison Report Between M78530_PEA_{—}1_P15 (SEQ ID NO:619) and Q9HCB6 (SEQ ID NO:617):

1. An isolated chimeric polypeptide encoding for M78530_PEA_{—}1_P15 (SEQ ID NO:619), comprising a first amino acid sequence being at least 90% homologous to MRLSPAPLKLSRTPALLALALPLAAALAFSDETLDKVPKSEGYCSRILRAQGTRREGYTEFSLRVEG DPDFYKPGTSYRVTLSAAPPSYFRGFTLIALRENREGDKEEDHAGTFQIIDEEETQFMSNCPVAVTE STPRRRTRIQVFWIAPPAGTGCVILKASIVQKRIIYFQDEGSLTKKLCEQDSTFDGVTDKPILDCCAC GTAKYRLTFYGNWSEKTHPKDYPRRANHWSAIIGGSHSKNYVLWEYGGYASEGVKQVAELGSPV KMEEEIRQQSDEVLTVIKAKAQWPAWQPLNVRAAPSAEFSVDRTRHLMSFLTMMGPSPDWNVGL SAEDLCTKECGWVQKVVQDLIPWDAGTDSGVTYESPNKPTIPQEKIRPLTSLDHPQSPFYDPEGGSI TQVARVVIERIARKGEQCNIVPDNVDDIVADLAPEEKDEDDTPETCIYSNWSPWSACSSSTCDKGK RMRQRMLKAQLDLSVPCPDTQDFQPCMGPGCSDEDGSTCTMSEWITWSPCSISCGMGMRSRERY VKQFPEDGSVCTLPTEE corresponding to amino acids 1-544 of Q9HCB6 (SEQ ID NO:617), which also corresponds to amino acids 1-544 of M78530_PEA_{—}1_P15 (SEQ ID NO:619), a bridging amino acid T corresponding to amino acid 545 of M78530_PEA_{—}1_P15 (SEQ ID NO:619), a second amino acid sequence being at least 90% homologous to EKCTVNEECSPSSCLMTEWGEWDECSATCGMGMKKRHRMIKMNPADGSMCKAETSQAEKCMM PECHTIPCLLSPWSEWSDCSVTCGKGMRTRQRMLKSLAELGDCNEDLEQVEKCMLPEC corresponding to amino acids 546-665 of Q9HCB6 (SEQ ID NO:617), which also corresponds to amino acids 546-665 of M78530_PEA_{—}1_P15 (SEQ ID NO:619), and a third amino acid sequence being at least 70%, optionally at least 80%, preferably at least 85%, more preferably at least 90% and most preferably at least 95% homologous to a polypeptide having the sequence RKSWSSSRPITSMFLSPGSPEPASANTARS (SEQ ID NO: 667) corresponding to amino acids 666-695 of M78530_PEA_{—}1_P15 (SEQ ID NO:619), wherein said first amino acid sequence, bridging amino acid, second amino acid sequence and third amino acid sequence are contiguous and in a sequential order.

2. An isolated polypeptide encoding for a tail of M78530_PEA_{—}1_P15 (SEQ ID NO:619), comprising a polypeptide being at least 70%, optionally at least about 80%, preferably at least about 85%, more preferably at least about 90% and most preferably at least about 95% homologous to the sequence RKSWSSSRPITSMFLSPGSPEPASANTARS (SEQ ID NO: 667) in M78530_PEA_{—}1_P15 (SEQ ID NO:619).

Comparison report between M78530_PEA_{—}1_P15 (SEQ ID NO:619) and 094862 (SEQ ID NO:618):

1. An isolated chimeric polypeptide encoding for M78530_PEA_{—}1_P15 (SEQ ID NO:619), comprising a first amino acid sequence being at least 70%, optionally at least 80%, preferably at least 85%, more preferably at least 90% and most preferably at least 95% homologous to a polypeptide having the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) corresponding to amino acids 1-83 of M78530_PEA_{—}1_P15 (SEQ ID NO:619), a second amino acid sequence being at least 90% homologous to AAPPSYFRGFTLIALRENREGDKEEDHAGTFQIIDEEETQFMSNCPVAVTESTPRRRTRIQVFWIAPP AGTGCVILKASIVQKRIIYFQDEGSLTKKLCEQDSTFDGVTDKPILDCCACGTAKYRLTFYGNWSE KTHPKDYPRRANHWSAIIGGSHSKNYVLWEYGGYASEGVKQVAELGSPVKMEEEIRQQSDEVLT VIKAKAQWPAWQPLNVRAAPSAEFSVDRTRHLMSFLTMMGPSPDWNVGLSAEDLCTKECGWVQ KVVQDLIPWDAGTDSGVTYESPNKPTIPQEKIRPLTSLDHPQSPFYDPEGGSITQVARVVIERIARKG EQCNIVPDNVDDIVADLAPEEKDEDDTPETCIYSNWSPWSACSSSTCDKGKRMRQRMLKAQLDLS VPCPDTQDFQPCMGPGCSDEDGSTCTMSEWITWSPCSISCGMGMRSRERYVKQFPEDGSVCTLPT EETEKCTVNEECSPSSCLMTEWGEWDECSATCGMGMKKRHRMIKMNPADGSMCKAETSQAEKC MMPECHTIPCLLSPWSEWSDCSVTCGKGMRTRQRMLKSLAELGDCNEDLEQVEKCMLPEC corresponding to amino acids 1-582 of O94862 (SEQ ID NO:618), which also corresponds to amino acids 84-665 of M78530_PEA_{—}1_P15 (SEQ ID NO:619), and a third amino acid sequence being at least 70%, optionally at least 80%, preferably at least 85%, more preferably at least 90% and most preferably at least 95% homologous to a polypeptide having the sequence RKSWSSSRPITSMFLSPGSPEPASANTARS (SEQ ID NO: 667) corresponding to amino acids 666-695 of M78530_PEA_{—}1_P15 (SEQ ID NO:619), wherein said first amino acid sequence, second amino acid sequence and third amino acid sequence are contiguous and in a sequential order.

2. An isolated polypeptide encoding for a head of M78530_PEA_{—}1_P15 (SEQ ID NO:619), comprising a polypeptide being at least 70%, optionally at least about 80%, preferably at least about 85%, more preferably at least about 90% and most preferably at least about 95% homologous to the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) of M78530_PEA_{—}1_P15 (SEQ ID NO:619).

3. An isolated polypeptide encoding for a tail of M78530_PEA_{—}1_P15 (SEQ ID NO:619), comprising a polypeptide being at least 70%, optionally at least about 80%, preferably at least about 85%, more preferably at least about 90% and most preferably at least about 95% homologous to the sequence RKSWSSSRPITSMFLSPGSPEPASANTARS (SEQ ID NO: 667) in M78530_PEA_{—}1_P15 (SEQ ID NO:619).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein M78530_PEA_{—}1_P15 (SEQ ID NO:619) also has the following non-silent SNPs (Single Nucleotide Polymorphisms) as listed in Table 252, (given according to their position(s) on the amino acid sequence, with the alternative amino acid(s) listed; the last column indicates whether the SNP is known or not; the presence of known SNPs in variant protein M78530_PEA_{—}1_P15 (SEQ ID NO:619) sequence provides support for the deduced sequence of this variant protein according to the present invention).

TABLE 252
Amino acid mutations

| SNP position(s) on amino | Alternative amino | |
|---|---|---|
| acid sequence | acid(s) | Previously known SNP? |
| 278 | E -> D | No |
| 278 | E -> V | No |

Variant protein M78530_PEA_{—}1_P15 (SEQ ID NO:619) is encoded by the following transcript(s): M78530_PEA_{—}1_T11 (SEQ ID NO:592). The coding portion of transcript M78530_PEA_{—}1_T11 (SEQ ID NO:592) starts at position 629 and ends at position 2713. The transcript also has the following SNPs as listed in Table 253 (given according to their position on the nucleotide sequence, with the alternative nucleic acid listed; the last column indicates whether the SNP is known or not; the presence of known SNPs in variant protein M78530_PEA_{—}1_P15 (SEQ ID NO:619) sequence provides support for the deduced sequence of this variant protein according to the present invention).

TABLE 253
Nucleic acid SNPs

| SNP position on nucleotide | Alternative | |
|---|---|---|
| sequence | nucleic acid | Previously known SNP? |
| 760 | C -> T | No |
| 1461 | A -> T | No |
| 1462 | G -> T | No |
| 1492 | A -> G | No |

Variant protein M78530_PEA_{—}1_P16 (SEQ ID NO:620) according to the present invention has an amino acid sequence; it is encoded by transcript(s) M78530_PEA_{—}1_T12 (SEQ ID NO:593). One or more alignments to one or more previously published protein sequences are given in the alignment table located on the attached CDROM. A brief description of the relationship of the variant protein according to the present invention to each such aligned protein is as follows:

Comparison Report Between M78530_PEA_{—}1_P16 (SEQ ID NO:620) and Q8NCD7 (SEQ ID NO:616):

1. An isolated chimeric polypeptide encoding for M78530_PEA_{—}1_P16 (SEQ ID NO:620), comprising a first amino acid sequence being at least 90% homologous to MRLSPAPLKLSRTPALLALALPLAAALAFSDETLDKVPKSEGYCSRILRAQGTRREGYTEFSLRVEG DPDFYKPGTSYRVTLSAAPPSYFRGFTLIALRENREGDKEEDHAGTFQIIDEEETQFMSNCPVAVTE STPRRRTRIQVFWIAPPAGTGCVILKASIVQKRIIYFQDEGSLTKKLCEQDSTFDGVTDKPILDCCAC GTAKYRLTFYGNWSEKTHPKDYPRRANHWSAIIGGSHSKNYVLWEYGGYASEGVKQVAELGSPV KMEEEIRQQSDEVLTVIKAKAQWPAWQPLNV corresponding to amino acids 1-297 of Q8NCD7 (SEQ ID NO:616), which also corresponds to amino acids 1-297 of M78530_PEA_{—}1_P16 (SEQ ID NO:620).

Comparison Report Between M78530_PEA_{—}1_P16 (SEQ ID NO:620) and Q9HCB6 (SEQ ID NO:617):

1. An isolated chimeric polypeptide encoding for M78530_PEA_{—}1_P16 (SEQ ID NO:620), comprising a first amino acid sequence being at least 90% homologous to MRLSPAPLKLSRTPALLALALPLAAALAFSDETLDKVPKSEGYCSRILRAQGTRREGYTEFSLRVEG DPDFYKPGTSYRVTLSAAPPSYFRGFTLIALRENREGDKEEDHAGTFQIIDEEETQFMSNCPVAVTE STPRRRTRIQVFWIAPPAGTGCVILKASIVQKRIIYFQDEGSLTKKLCEQDSTFDGVTDKPILDCCAC GTAKYRLTFYGNWSEKTHPKDYPRRANHWSAIIGGSHSKNYVLWEYGGYASEGVKQVAELGSPV KMEEEIRQQSDEVLTVIKAKAQWPAWQPLNV corresponding to amino acids 1-297 of Q9HCB6 (SEQ ID NO:617), which also corresponds to amino acids 1-297 of M78530_PEA_{—}1_P16 (SEQ ID NO:620).

Comparison Report Between M78530_PEA_{—}1_P16 (SEQ ID NO:620) and O94862 (SEQ ID NO:618):

1. An isolated chimeric polypeptide encoding for M78530_PEA_{—}1_P16 (SEQ ID NO:620), comprising a first amino acid sequence being at least 70%, optionally at least 80%, preferably at least 85%, more preferably at least 90% and most preferably at least 95% homologous to a polypeptide having the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) corresponding to amino acids 1-83 of M78530_PEA_{—}1_P16 (SEQ ID NO:620), and a second amino acid sequence being at least 90% homologous to AAPPSYFRGFTLIALRENREGDKEEDHAGTFQIIDEEETQFMSNCPVAVTESTPRRRTRIQVFWIAPP AGTGCVILKASIVQKRIIYFQDEGSLTKKLCEQDSTFDGVTDKPILDCCACGTAKYRLTFYGNWSE KTHPKDYPRRANHWSAIIGGSHSKNYVLWEYGGYASEGVKQVAELGSPVKMEEEIRQQSDEVLT VIKAKAQWPAWQPLNV corresponding to amino acids 1-214 of O94862 (SEQ ID NO:618), which also corresponds to amino acids 84-297 of M78530_PEA_{—}1_P16 (SEQ ID NO:620), wherein said first amino acid sequence and second amino acid sequence are contiguous and in a sequential order.

2. An isolated polypeptide encoding for a head of M78530_PEA_{—}1_P16 (SEQ ID NO:620), comprising a polypeptide being at least 70%, optionally at least about 80%, preferably at least about 85%, more preferably at least about 90% and most preferably at least about 95% homologous to the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) of M78530_PEA_{—}1_P16 (SEQ ID NO:620).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein M78530_PEA_{—}1_P16 (SEQ ID NO:620) also has the following non-silent SNPs (Single Nucleotide Polymorphisms) as listed in Table 254, (given according to their position(s) on the amino acid sequence, with the alternative amino acid(s) listed; the last column indicates whether the SNP is known or not; the presence of known SNPs in variant protein M78530_PEA_{—}1_P16 (SEQ ID NO:620) sequence provides support for the deduced sequence of this variant protein according to the present invention).

TABLE 254
Amino acid mutations

| SNP position(s) on amino acid | Alternative | |
|---|---|---|
| sequence | amino acid(s) | Previously known SNP? |
| 278 | E -> D | No |
| 278 | E -> V | No |

Variant protein M78530_PEA_{—}1_P16 (SEQ ID NO:620) is encoded by the following transcript(s): M78530_PEA_{—}1_T12 (SEQ ID NO:593). The coding portion of transcript M78530_PEA_{—}1_T12 (SEQ ID NO:593) starts at position 629 and ends at position 1519. The transcript also has the following SNPs as listed in Table 255 (given according to their position on the nucleotide sequence, with the alternative nucleic acid listed; the last column indicates whether the SNP is known or not; the presence of known SNPs in variant protein M78530_PEA_{—}1_P16 (SEQ ID NO:620) sequence provides support for the deduced sequence of this variant protein according to the present invention).

TABLE 255
Nucleic acid SNPs

| SNP position on | | |
|---|---|---|
| nucleotide sequence | Alternative nucleic acid | Previously known SNP? |
| 760 | C -> T | No |
| 1461 | A -> T | No |
| 1462 | G -> T | No |
| 1492 | A -> G | No |
| 1670 | T -> C | No |
| 1957 | T -> C | No |
| 2004 | A -> C | No |
| 2005 | A -> T | No |

Variant protein M78530 PEA_{—}1_P17 (SEQ ID NO:621) according to the present invention has an amino acid sequence it is encoded by transcript(s) M78530_PEA_{—}1_T13 (SEQ ID NO:594). One or more alignments to one or more previously published protein sequences are given in the alignment table located on the attached CDROM. A brief description of the relationship of the variant protein according to the present invention to each such aligned protein is as follows:

Comparison Report Between M78530_PEA_{—}1_P17 (SEQ ID NO:621) and Q8NCD7 (SEQ ID NO:616):

1. An isolated chimeric polypeptide encoding for M78530_PEA_{—}1_P17 (SEQ ID NO:621), comprising a first amino acid sequence being at least 90% homologous to MRLSPAPLKLSRTPALLALALPLAAALAFSDETLDKVPKSEGYCSRILRAQGTRREGYTEFSLRVEG DPDFYKPGTSYRVTLSAAPPSYFRGFTLIALRENREGDKEEDHAGTFQIIDEEETQFMSNCPVAVTE STPRRRTRIQVFWIAPPAGTGCVILKASIVQKRIIYFQDEGSLTKKLCEQDSTFDGVTDKPILDCCAC GTAKYRLTFYGNWSEKTHPKDYPRRANHWSAIIGGSHSKNYVLWEYGGYASEGVKQVAELGSPV KMEEEIRQQ corresponding to amino acids 1-275 of Q8NCD7 (SEQ ID NO:616), which also corresponds to amino acids 1-275 of M78530_PEA_{—}1_P17 (SEQ ID NO:621), and a second amino acid sequence being at least 70%, optionally at least 80%, preferably at least 85%, more preferably at least 90% and most preferably at least 95% homologous to a polypeptide having the sequence VRQKNHRMTK (SEQ ID NO: 670) corresponding to amino acids 276-285 of M78530_PEA_{—}1_P17 (SEQ ID NO:621), wherein said first amino acid sequence and second amino acid sequence are contiguous and in a sequential order.

2. An isolated polypeptide encoding for a tail of M78530_PEA_{—}1_P17 (SEQ ID NO:621), comprising a polypeptide being at least 70%, optionally at least about 80%, preferably at least about 85%, more preferably at least about 90% and most preferably at least about 95% homologous to the sequence VRQKNHRMTK (SEQ ID NO: 670) in M78530_PEA_{—}1_P17 (SEQ ID NO:621).

Comparison Report Between M78530_PEA_{—}1_P17 (SEQ ID NO:621) and Q9HCB6 (SEQ ID NO:617):

1. An isolated chimeric polypeptide encoding for M78530_PEA_{—}1_P17 (SEQ ID NO:621), comprising a first amino acid sequence being at least 90% homologous to MRLSPAPLKLSRTPALLALALPLAAALAFSDETLDKVPKSEGYCSRILRAQGTRREGYTEFSLRVEG DPDFYKPGTSYRVTLSAAPPSYFRGFTLIALRENREGDKEEDHAGTFQIIDEEETQFMSNCPVAVTE STPRRRTRIQVFWIAPPAGTGCVILKASIVQKRIIYFQDEGSLTKKLCEQDSTFDGVTDKPILDCCAC GTAKYRLTFYGNWSEKTHPKDYPRRANHWSAIIGGSHSKNYVLWEYGGYASEGVKQVAELGSPV KMEEEIRQQ corresponding to amino acids 1-275 of Q9HCB6 (SEQ ID NO:617), which also corresponds to amino acids 1-275 of M78530_PEA_{—}1_P17 (SEQ ID NO:621), and a second amino acid sequence being at least 70%, optionally at least 80%, preferably at least 85%, more preferably at least 90% and most preferably at least 95% homologous to a polypeptide having the sequence VRQKNHRMTK (SEQ ID NO: 670) corresponding to amino acids 276-285 of M78530 PEA_{—}1_P17 (SEQ ID NO:621), wherein said first amino acid sequence and second amino acid sequence are contiguous and in a sequential order.

2. An isolated polypeptide encoding for a tail of M78530_PEA_{—}1_P17 (SEQ ID NO:621), comprising a polypeptide being at least 70%, optionally at least about 80%, preferably at least about 85%, more preferably at least about 90% and most preferably at least about 95% homologous to the sequence VRQKNHRMTK (SEQ ID NO: 670) in M78530_PEA_{—}1_P17 (SEQ ID NO:621).

Comparison Report Between M78530_PEA_{—}1_P17 (SEQ ID NO:621) and O94862 (SEQ ID NO:618):

1. An isolated chimeric polypeptide encoding for M78530_PEA_{—}1_P17 (SEQ ID NO:621), comprising a first amino acid sequence being at least 70%, optionally at least 80%, preferably at least 85%, more preferably at least 90% and most preferably at least 95% homologous to a polypeptide having the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) corresponding to amino acids 1-83 of M78530_PEA_{—}1_P17 (SEQ ID NO:621), a second amino acid sequence being at least 90% homologous to AAPPSYFRGFTLIALRENREGDKEEDHAGTFQIIDEEETQFMSNCPVAVTESTPRRRTRIQVFWIAPP AGTGCVILKASIVQKRIIYFQDEGSLTKKLCEQDSTFDGVTDKPILDCCACGTAKYRLTFYGNWSE KTHPKDYPRRANHWSAIIGGSHSKNYVLWEYGGYASEGVKQVAELGSPVKMEEEIRQQ corresponding to amino acids 1-192 of O94862 (SEQ ID NO:618), which also corresponds to amino acids 84-275 of M78530_PEA_{—}1_P17 (SEQ ID NO:621), and a third amino acid sequence being at least 70%, optionally at least 80%, preferably at least 85%, more preferably at least 90% and most preferably at least 95% homologous to a polypeptide having the sequence VRQKNHRMTK (SEQ ID NO: 670) corresponding to amino acids 276-285 of M78530_PEA_{—}1_P17 (SEQ ID NO:621), wherein said first amino acid sequence, second amino acid sequence and third amino acid sequence are contiguous and in a sequential order.

2. An isolated polypeptide encoding for a head of M78530_PEA_{—}1_P17 (SEQ ID NO:621), comprising a polypeptide being at least 70%, optionally at least about 80%, preferably at least about 85%, more preferably at least about 90% and most preferably at least about 95% homologous to the sequence MRLSPAPLKLSRTPALLALALPLAAALAFSDETLDKVPKSEGYCSRILRAQGTRREGYTEFSLRVEG DPDFYKPGTSYRVTLS (SEQ ID NO: 668) of M78530_PEA_{—}1_P17 (SEQ ID NO:621).

3. An isolated polypeptide encoding for a tail of M78530_PEA_{—}1_P17 (SEQ ID NO:621), comprising a polypeptide being at least 70%, optionally at least about 80%, preferably at least about 85%, more preferably at least about 90% and most preferably at least about 95% homologous to the sequence VRQKNHRMTK (SEQ ID NO: 670) in M78530_PEA_{—}1_P17 (SEQ ID NO:621).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein M78530_PEA_{—}1_P17 (SEQ ID NO:621) is encoded by the following transcript(s): M78530_PEA_{—}1_T13 (SEQ ID NO:594). The coding portion of transcript M78530_PEA_{—}1_T13 (SEQ ID NO:594) starts at position 629 and ends at position 1483. The transcript also has the following SNPs as listed in Table 256 (given according to their position on the nucleotide sequence, with the alternative nucleic acid listed; the last column indicates whether the SNP is known or not; the presence of known SNPs in variant protein M78530_PEA_{—}1_P17 (SEQ ID NO:621) sequence provides support for the deduced sequence of this variant protein according to the present invention).

TABLE 256
Nucleic acid SNPs

| SNP position on nucleotide | Alternative | |
|---|---|---|
| sequence | nucleic acid | Previously known SNP? |
| 760 | C -> T | No |

Table 257 below describes the starting and ending position of M78530_PEA_{—}1_node_{—}37 (SEQ ID NO:604) on the relevant transcript. Experimental results for this segment are described below.

TABLE 257
Segment location on transcripts

| | Segment | |
|---|---|---|
| | starting | Segment |
| Transcript name | position | ending position |
| M78530_PEA_1_T11 (SEQ ID NO: 592) | 2625 | 2816 |

Expression of Spondin 1 M78530 transcripts which are detectable by amplicon as depicted in sequence name M78530seg37 (SEQ ID NO: 624) in normal and cancerous ovary tissues

Expression of Spondin 1 transcripts detectable by or according to seg37-M78530 seg37 (SEQ ID NO: 624) amplicon and primers M78530seg37F (SEQ ID NO: 622) and M78530seg37R (SEQ ID NO: 623) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and GAPDH (GenBank Accession No. BC026907 (SEQ ID NO:3); GAPDH amplicon (SEQ ID NO: 41)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold up-regulation for each sample relative to median of the normal PM samples.

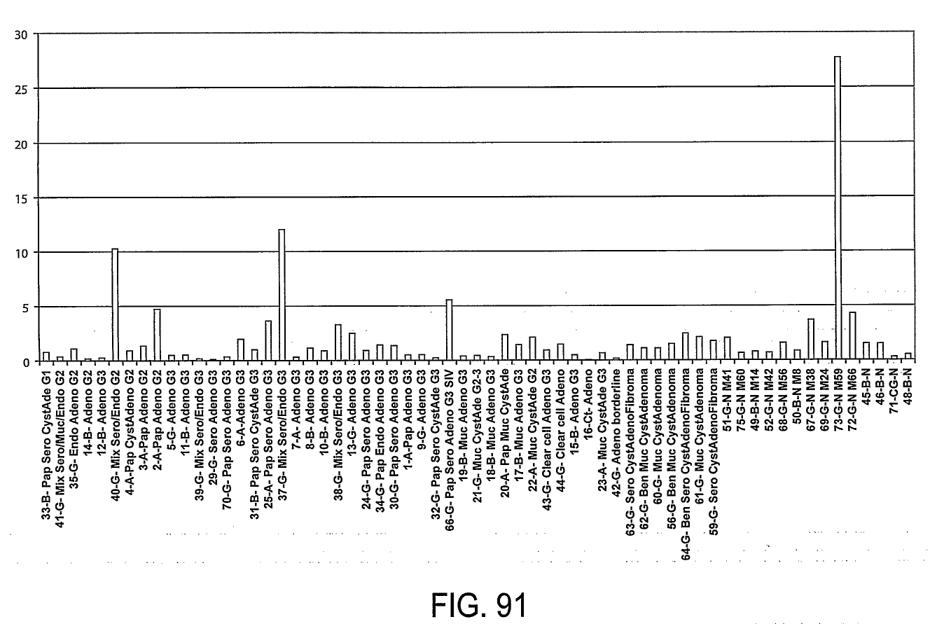

FIG. 91 is a histogram showing over expression of the above-indicated Spondin 1 transcripts in cancerous ovary samples relative to the normal samples.

As is evident from FIG. 91, the expression of Spondin 1 transcripts detectable by the above amplicon was higher in a few cancer samples than in the non-cancerous samples (Sample Nos. 45, 46, 48, 71, Table 2, “Tissue samples in ovarian cancer testing panel”. Notably an over-expression of at least 5 fold was found in 3 out of 40 adenocarcinoma samples.

Primer pairs are also optionally and preferably encompassed within the present invention; for example, for the above experiment, the following primer pair was used as a non-limiting illustrative example only of a suitable primer pair: M78530seg37F (SEQ ID NO: 622) forward primer; and M78530seg37R (SEQ ID NO: 623) reverse primer.

The present invention also preferably encompasses any amplicon obtained through the use of any suitable primer pair; for example, for the above experiment, the following amplicon was obtained as a non-limiting illustrative example only of a suitable amplicon: M78530seg37 (SEQ ID NO: 624).

Primers:

| | |
|---|---|
| Forward primer M78530seg37F: | (SEQ ID NO: 622) |
| AGCTCCTCAAGGCCCATCA | |
| Reverse primer M78530seg37R: | (SEQ ID NO: 623) |
| AACTCTTCCCCAGCAAACCAT | |
| Amplicon M78530seg37: | (SEQ ID NO: 624) |

AGCTCCTCAAGGCCCATCACTTCTATGTTCCTGAGTCCAGGGAGCCCCGAACCAGCCAGTGCT
AATACTGCTAGATCCTAGAATAACATGGTTTGCTGGGGAAGAGTT

Expression of Spondin 1 M78530 transcripts which are detectable by amplicon as depicted in sequence name M78530seg40WT (SEQ ID NO: 627) in normal and cancerous ovary tissues

Expression of Spondin 1 transcripts detectable by or according to seg40WT-M78530 Seg40WT (SEQ ID NO: 627) amplicon and primers M78530seg40WTF (SEQ ID NO: 625) and M78530seg40WTR (SEQ ID NO: 626) was measured by real time PCR. In parallel the expression of four housekeeping genes—PBGD (GenBank Accession No. BC019323 (SEQ ID NO:6); amplicon—PBGD-amplicon (SEQ ID NO:32)), HPRT1 (GenBank Accession No. NM_{—}000194 (SEQ ID NO:5); amplicon—HPRT1-amplicon (SEQ ID NO:35)), SDHA (GenBank Accession No. NM_{—}004168 (SEQ ID NO:4); amplicon—SDHA-amplicon (SEQ ID NO:29)), and GAPDH (GenBank Accession No. BC026907 (SEQ ID NO:3); GAPDH amplicon (SEQ ID NO: 41)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 45, 46, 48, 71, Table 3, above, “Tissue samples in ovarian cancer testing panel”), to obtain a value of fold up-regulation for each sample relative to median of the normal PM samples.

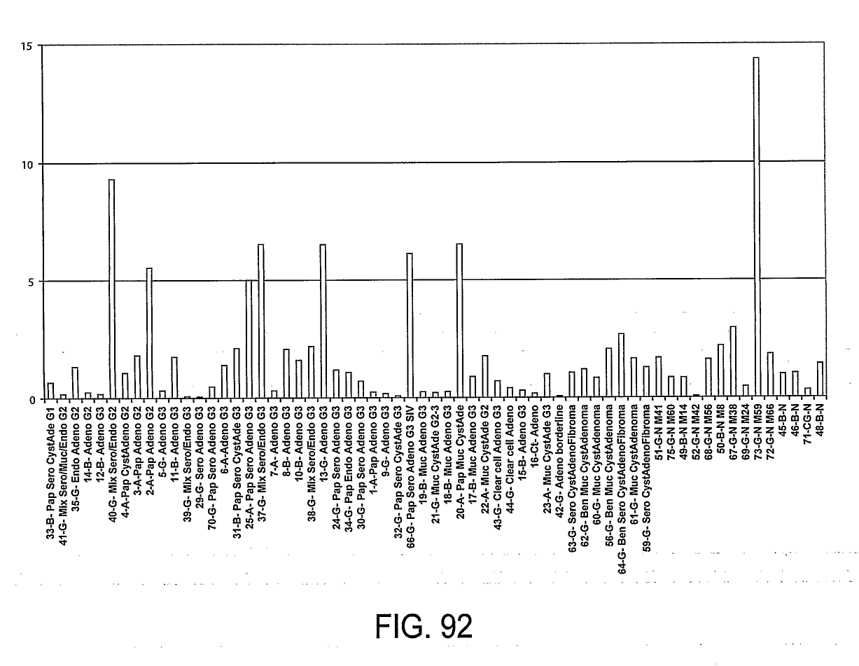

FIG. 92 is a histogram showing over expression of the above-indicated Spondin 1 transcripts in cancerous ovary samples relative to the normal samples.

As is evident from FIG. 92, the expression of Spondin 1 transcripts detectable by the above amplicon was higher in a few cancer samples than in the non-cancerous samples (Sample Nos. 45, 46, 48, 71, Table 2, “Tissue samples in ovarian cancer testing panel”). Notably an over-expression of at least 5 fold was found in 7 out of 40 adenocarcinoma samples.

Primer pairs are also optionally and preferably encompassed within the present invention; for example, for the above experiment, the following primer pair was used as a non-limiting illustrative example only of a suitable primer pair: M78530seg40WTF (SEQ ID NO: 625) forward primer; and M78530seg40WTR (SEQ ID NO: 626) reverse primer.

The present invention also preferably encompasses any amplicon obtained through the use of any suitable primer pair; for example, for the above experiment, the following amplicon was obtained as a non-limiting illustrative example only of a suitable amplicon: M78530seg40WT (SEQ ID NO: 627). FIG. 91 is a histogram showing expression of Spondin 1 M78530 transcripts which are detectable by amplicon as depicted in sequence name M78530 seg40WT (SEQ ID NO: 627) in normal and cancerous ovary tissues

Primers:

| | |
|---|---|
| Forward primer M78530seg40WTF: | (SEQ ID NO: 625) |
| TCCCAGTGGTCGGAATGTAAC | |
| Reverse primer M78530seg40WT: | (SEQ ID NO: 626) |
| GCACTTTTTTCGCTGCACAG | |
| Amplicon M78530seg40WT: | (SEQ ID NO: 627) |

TCCCAGTGGTCGGAATGTAACAAGTCATGTGGGAAAGGCCACGTGATTCGAACCCGGATGAT
CCAAATGGAGCCTCAGTTTGGAGGTGCACCCTGCCCAGAGACTGTGCAGCGAAAAAAGTGC

Expression of spondin 1 transcripts which are detectable by junction of segments 2-4, in normal, benign and cancerous ovary tissues

Expression of spondin 1 transcripts detectable by or according to junction of segments 2-4, was measured with oligonucleotide-based micro-arrays. The results of image intensities for each feature were normalized according to the percentile 95 of the image intensities of all the features on the chip. Then, feature image intensities for replicates of the same oligonucleotide on the chip and replicates of the same sample were averaged. Outlying results were discarded.

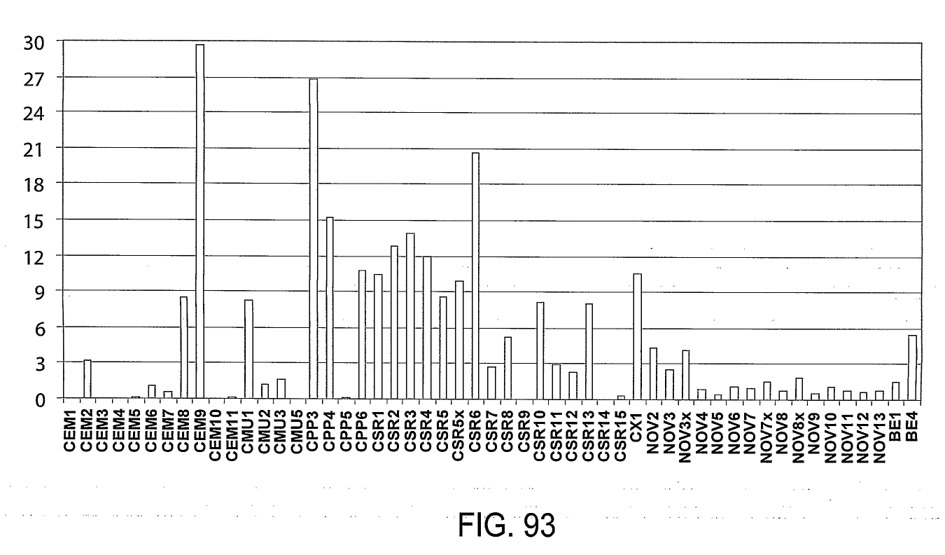

For oligonucleotide M78530_{—}0_{—}27_{—}120 (SEQ ID NO:14) the averaged intensity determined for every sample was divided by the median intensity of all the normal samples (Samples November 2-13, Table 258, below), to obtain a value of fold up-regulation for each sample relative to the median normal samples. These data are presented in a histogram bellow. As is evident from the histogram, the expression of spondin 1 transcripts detectable with the above oligonucleotides in cancer samples was significantly higher than in the normal samples. FIG. 93 is a histogram showing Expression of spondin 1 transcripts which are detectable by junction of segments 2-4, in normal, benign and cancerous ovary tissues.

| | |
|---|---|
| >M78530_0_27_120 | (SEQ ID NO: 14) |

CCAGATCATAGACGAAGAAGAAACTCAGTTTATGAGCAATTGCCCTGTTG

TABLE 258

| | TAA2_MA | | | | | | |
|---|---|---|---|---|---|---|---|
| Sample # | ID | Tissue ID | RNA ID | Source | age | Stage | grade |
| | | OvSr = SEROUS | | | | | |
| | | ADENOCARCINOMA | | | | | |
| 1 | OvSr1 | 2O37OAI3 | 2O37ORTX | GCI | 43 | II | |
| 2 | OvSr2 | 3NTISA77 | 3NTISRY4 | GCI | 53 | III | |
| 3 | OvSr3 | 4WAABA68 | 4WAABR62 | GCI | 63 | III | 1 |
| 4 | OvSr4 | 79Z67AL4 | 79Z67RFA | GCI | 67 | III | |
| 5 | OvSr5 | 7B3DPA5S | 7B3DPR3Y | GCI | 70 | II | 2 |
| 6 | OvSr6 | 7RMHZAMG | 7RMHZRQ9 | GCI | 63 | III | 3 |
| 7 | OvSr7 | CEJUSAVO | CEJUSRZG | GCI | 53 | III | 3 |
| 8 | OvSr8 | DDSNLAWD | DDSNLR79 | GCI | 68 | III | 3 |
| 9 | OvSr9 | DH8PHAMR | DH8PHRPE | GCI | 70 | IV | 3 |
| 10 | OvSr10 | 5NCLKA15 | 5NCLKR2O | GCI | 54 | III | |
| 11 | OvSr11 | 1HI5HAHH | 1HI5HRE2 | GCI | 61 | III | |
| 12 | OvSr12 | 33-B-Pap Sero CystAde G1 | A503175 | BioChain | 41 | | 1 |
| 13 | OvSr13 | 31-B-Pap Sero CystAde G3 | A503176 | BioChain | 52 | | 3 |
| 14 | OvSr14 | 29-G-Sero Adeno G3 | 2001-12-G035 | GOG | 50 | | 3 |
| 15 | OvSr15 | 9-G-Adeno G3 | 99-06-G901 | GOG | 84 | | 3 |
| 66 | OvSr16 | 18701 | 40773C1 | Asterand | 59 | IIA | 2 |
| 67 | OvSr17 | 13268 | 19832A1 | Asterand | 48 | IIIC | high |
| | | | | | | | grade |
| | | OvPp = Papillary | | | | | |
| | | adenocarcinoma | | | | | |
| 16 | OvPp1 | 4-A-Pap CystAdeno G2 | ILS-7286 | ABS | 50 | | 2 |
| 17 | OvPp2 | 3-A-Pap Adeno G2 | ILS-1431 | ABS | 52 | | 2 |
| 18 | OvPp3 | 2-A-Pap Adeno G2 | ILS-1408 | ABS | 53 | | 2 |
| 19 | OvPp4 | 25-A-Pap Sero Adeno G3 | N0021 | ABS | 55 | | 3 |
| 20 | OvPp5 | 1-A-Pap Adeno G3 | ILS-1406 | ABS | 73 | | 3 |
| 21 | OvPp6 | 66-G-Pap Sero Adeno G3 SIV | 2000-01-G413 | GOG | 67 | | 3 |
| | | OvEm = ENDOMETROID | | | | | |
| | | ADENOCARINOMA | | | | | |
| 22 | OvEm1 | 1U52XAHJ | 1U52XRPE | GCI | 61 | II | 3 |
| 23 | OvEm2 | 533DXAHE | 533DXRKV | GCI | 50 | I | |
| 24 | OvEm3 | 5895CAXD | 5895CR56 | GCI | 39 | I | |
| 25 | OvEm4 | A17WSACA | A17WSR7Y | GCI | 67 | II | 2 |
| 26 | OvEm5 | E2WKFA4F | E2WKFRPT | GCI | 30 | I | 1 |
| 27 | OvEm6 | HZ2EYAU6 | HZ2EYRC6 | GCI | 55 | I | 3 |
| 28 | OvEm7 | PZQXHALS | PZQXHRGN | GCI | 52 | III | 1 |
| 29 | OvEm8 | RWOIVALL | RWOIVRI1 | GCI | 47 | II | |
| 30 | OvEm9 | 1VT3IAZ6 | 1VT3IRT1 | GCI | 50 | III | 3 |
| 31 | OvEm10 | I8VHZALI | I8VHZRR4 | GCI | 68 | IV | 3 |
| 32 | OvEm11 | 34-G-Pap Endo Adeno G3 | 95-04-2002 | GOG | 68 | | 3 |
| | | OvMu = Mucinous | | | | | |
| | | adenocarcinoma and mucinus | | | | | |
| | | borderline | | | | | |
| | | Mucinus adenocarcinoma | | | | | |
| 33 | OvMu1 | 22-A-Muc CystAde G2 | A0139 | ABS | 72 | | 2 |
| 35 | OvMu6 | 19-B-Muc Adeno G3 | A504085 | BioChain | 34 | | 3 |
| 36 | OvMu3 | 17-B-Muc Adeno G3 | A504084 | BioChain | 51 | | 3 |
| 37 | OvMu5 | IMDA1ANG | IMDA1RQG | GCI | 41 | I | 3 |
| 38 | OvMu2 | 21-G-Muc CystAde G2-3 | 95-10-G020 | GOG | 44 | | 2 to 3 |
| 68 | OvMu7 | 12742 | 18920A1 | Asterand | 61 | IC | |
| 69 | OvMu8 | NJM4UAC4 | NJM4URI5 | GCI | 51 | | |
| | | Mucinus borderline tumors | | | | | |
| 70 | OvMu9_BL | 3D5FOA9R | 3D5FORJ9 | GCI | 51 | 1A | |
| 71 | OvMu10_BL | 7JP3FAIH | 7JP3FRCY | GCI | 56 | 1A | 1 |
| 72 | OvMu11_BL | SC656AKT | SC656RN6 | GCI | 40 | 1A | |
| 34 | OvMu4 | 23-A-Muc CystAde G3 | VNM-00187 | ABS | 45 | | |
| | | OvBe = Benign samples | | | | | |
| 39 | OvBe1 | 62-G-Ben Muc CysAdenoma | 99-10-G442 | GOG | 32 | | |
| 40 | OvBe2 | 60-G-Muc CysAdenoma | 99-01-G043 | GOG | 40 | | |
| 41 | OvBe3 | 56-G-Ben Muc CysAdeno | 99-01-G407 | GOG | 46 | | |
| 42 | OvBe4 | 64-G-Ben Sero CysAdenoma | 99-06-G039 | GOG | 57 | | |
| 43 | OvBe5 | 59-G-Sero CysAdenoFibroma | 98-12-G401 | GOG | 77 | | |
| 44 | OvBe6 | QLIKYAKS | QLIKYRNG | GCI | 42 | | |
| 45 | OvBe7 | 943ECATN | 943ECRVO | GCI | 54 | | |
| 46 | OvBe8 | 943ECAW7 | 943ECRYK | GCI | 54 | | |
| 47 | OvBe9 | JO8W7AKQ | JO8W7RTI | GCI | 56 | | |
| 48 | OvBe10 | DQQ2FAMC | DQQ2FRAC | GCI | 68 | | |
| | | NOv = Normal Samples | | | | | |
| 49 | NOv1 | 45-B-N | A503274 | BioChain | 41 | | |
| 50 | NOv2 | 46-B-N | A504086 | BioChain | 41 | | |
| 51 | NOv3 | 48-B-N | A504087 | BioChain | 51 | | |
| 52 | NOv4 | GWXUZN5M | GWXUZRI3 | GCI | 53 | | |
| 53 | NOv5 | IDUVYN9I | IDUVYROT | GCI | 47 | | |
| 54 | NOv6 | L629FN58 | L629FRV1 | GCI | 47 | | |
| 55 | NOv7 | SJ2R2NPS | SJ2R2RFN | GCI | 43 | | |
| 56 | NOv8 | TW9PMN69 | TW9PMR25 | GCI | 46 | | |
| 57 | NOv9 | XLB23NA4 | XLB23RKV | GCI | 47 | | |
| 58 | NOv10 | DWHTZNBF | DWHTZRQX | GCI | 42 | | |
| 59 | NOv11 | FDPL9NJ6 | FDPL9RVC | GCI | 56 | | |
| 60 | NOv12 | TOAE5N2M | TOAE5R37 | GCI | 45 | | |
| 61 | NOv13 | DD73BNIO | DD73BR3V | GCI | 49 | | |
| | | OvExtr = Clear cell & other | | | | | |
| | | samples | | | | | |
| 62 | OvExtr1 | 41-G-Mix Sero/Muc/Endo G2 | 98-03-G803 | GOG | 38 | | 2 |
| 63 | OvExtr2 | 43-G-Clear cell Adeno G3 | 2001-10-G002 | GOG | 74 | | 3 |
| 64 | OvExtr3 | 44-G-Clear cell Adeno | 2001-07-G084 | GOG | 73 | | |
| 65 | OvExtr4 | 42-G-Adeno borderline | 98-08-G001 | GOG | 46 | | |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-94. (canceled)

95. An isolated polynucleotide comprising the polynucleotide sequence set forth in a member selected from the group consisting of SEQ ID NOs: 10, 11, 13, 26, 93, 96, 99, 120, 123, 126, 144, 147, 150-163, 247, 250-315, 325, 328, 331, 334, 337-362, 370, 375, 378, 381, 384, 387-399, 407, 410-425, 432-462, 471, 474, 477, 550, 552, 557, 560, 563, 585, 588, 591, 624, 627, 630 and 698, or a sequence at least about 95% identical thereto.

96. An isolated polypeptide comprising the polypeptide sequence set forth in a member selected from the group consisting of SEQ ID NOs: 165, 166, 317-322, 364-369, 401-404, 427-429, 464-468, 642, 643, 650-664, or a sequence at least about 95% identical thereto.

97. An expression vector comprising the polynucleotide sequence according to claim 95.

98. A host cell comprising the vector of claim 97.

99. A process for producing a polypeptide comprising: culturing the host cell according to claim 98 under conditions suitable to produce the polypeptide encoded by said polynucleotide; and recovering said polypeptide.

100. An isolated primer pair, comprising the pair of nucleic acid sequences selected from the group consisting of: SEQ NOs. 91-92, 94-95, 97-98, 118-119, 121-122, 124-125, 142-143, 145-146, 148-149, 245-246, 248-249, 323-324, 326-327, 329-330, 332-333, 335-336, 371-374, 376-377, 379-380, 382-383, 385-386, 405-406, 408-409, 430-431, 469-470, 472-473, 475-476, 548-549, 551, 553-556, 558-559, 561-562, 583-584, 586-587, 589-590, 622-623, 625-626, and 699-701.

101. An antibody to specifically bind to the polypeptide of claim 96.

102. A kit for detecting a marker-detectable disease, comprising at least one primer pair of claim 100.

103. The kit of claim 102, wherein the marker-detectable disease is cancer, and is selected from the group consisting of ovarian cancer, breast cancer, colon cancer, lung cancer and prostate cancer, and wherein the cancer is invasive or metastatic.

104. The kit of claim 102, wherein the marker-detectable disease is heart disorder, cardiac disease or cardiac pathology, and is selected from the group consisting of: Myocardial infarction; ungina pectoris (stable and unstable); cardiomyopathy; myocarditis; heart failure; congestive heart failure; the detection of reinfarction; the detection of success of thrombolytic therapy after Myocardial infarct; Myocardial infarct after surgery, or assessing the size of infarct in Myocardial infarct; acute coronary syndrome; the differential diagnosis of heart related conditions from lung related conditions (as pulmonary embolism); the differential diagnosis of Dyspnea; cardiac valves related conditions; sudden cardiac death from arrhythmia or any other heart related reason; rejection of a transplanted heart; atrial and ventricular septal defects; conditions that cause atrial and or ventricular wall volume overload, systemic arterial hypertension.

105. The kit of claim 102, wherein the marker-detectable disease is cerebrovascular disease, and is selected from the group consisting of: stroke; ischemic stroke; thrombotic stroke; embolic stroke; lacunar stroke; hypoperfusion stroke; hemorrhagic stroke; transient ischemic attacks; neural tissue injury; brain trauma; and any type of cerebrovascular accident.

106. The kit of claim 102, wherein the marker-detectable disease is acute and chronic inflammation, and is selected from the group consisting of hypercholesterolemia, diabetes, atherosclerosis, acute or chronic inflammation that involves blood vessels, including coronary arteries and blood vessels of the brain, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behçet syndrome, and their complications including coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina; rheumatic/autoimmune diseases that involve systemic immune reaction including rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus; acute or chronic infective processes that involve systemic immune reaction, including pneumonia, bacteremia, sepsis, pyelonephritis, cellulitis, osteomyelitis, meningitis and viral hepatitis; malignant and idiopathic processes that involve systemic immune reaction or proliferation of immune cells, including granulomatous disorders, Wegener's granulomatosis, lymphomatoid granulomatosis/polymorphic reticulosis, idiopathic midline granuloma, multiple myeloma, Waldenstrom's macroglobulinemia, Castleman's disease, histiocytosis.

107. A kit for detecting a marker-detectable disease, comprising the antibody of claim 101.

108. The kit of claim 107, wherein said kit further comprises at least one immunoassay reagent.

109. The kit of claim 108, wherein said immunoassay is selected from the group consisting of an enzyme linked immunosorbent assay (ELISA), an immunoprecipitation assay, an immunofluorescence analysis, an enzyme immunoassay (EIA), a radioimmunoassay (RIA), or a Western blot analysis.

110. The kit of claim 107, wherein the marker-detectable disease is cancer, and is selected from the group consisting of ovarian cancer, breast cancer, colon cancer, lung cancer and prostate cancer, and wherein the cancer is invasive or metastatic.

111. The kit of claim 107, wherein the marker-detectable disease is heart disorder, cardiac disease or cardiac pathology, and is selected from the group consisting of: Myocardial infarction; ungina pectoris (stable and unstable); cardiomyopathy; myocarditis; heart failure; congestive heart failure; the detection of reinfarction; the detection of success of thrombolytic therapy after Myocardial infarct; Myocardial infarct after surgery, or assessing the size of infarct in Myocardial infarct; acute coronary syndrome; the differential diagnosis of heart related conditions from lung related conditions (as pulmonary embolism); the differential diagnosis of Dyspnea; cardiac valves related conditions; sudden cardiac death from arrhythmia or any other heart related reason; rejection of a transplanted heart; atrial and ventricular septal defects; conditions that cause atrial and or ventricular wall volume overload, systemic arterial hypertension.

112. The kit of claim 107, wherein the marker-detectable disease is cerebrovascular disease, and is selected from the group consisting of: stroke; ischemic stroke; thrombotic stroke; embolic stroke; lacunar stroke; hypoperfusion stroke; hemorrhagic stroke; transient ischemic attacks; neural tissue injury; brain trauma; and any type of cerebrovascular accident.

113. The kit of claim 107, wherein the marker-detectable disease is acute and chronic inflammation, and is selected from the group consisting of hypercholesterolemia, diabetes, atherosclerosis, acute or chronic inflammation that involves blood vessels, including coronary arteries and blood vessels of the brain, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behçet syndrome, and their complications including coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina; rheumatic/autoimmune diseases that involve systemic immune reaction including rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus; acute or chronic infective processes that involve systemic immune reaction, including pneumonia, bacteremia, sepsis, pyelonephritis, cellulitis, osteomyelitis, meningitis and viral hepatitis; malignant and idiopathic processes that involve systemic immune reaction or proliferation of immune cells, including granulomatous disorders, Wegener's granulomatosis, lymphomatoid granulomatosis/polymorphic reticulosis, idiopathic midline granuloma, multiple myeloma, Waldenstrom's macroglobulinemia, Castleman's disease, histiocytosis.

114. A method for detecting a marker-detectable disease, comprising detecting overexpression of the polynucleotide sequence set forth in a member selected from the group consisting of SEQ ID NOs: 10, 11, 13, 26, 93, 96, 99, 120, 123, 126, 144, 147, 150-163, 247, 250-315, 325, 328, 331, 334, 337-362, 370, 375, 378, 381, 384, 387-399, 407, 410-425, 432-462, 471, 474, 477, 550, 552, 557, 560, 563, 585, 588, 591, 624, 627, 630 and 698, or a sequence at least about 95% identical thereto in a sample from a patient.

115. The method of claim 114, wherein said detecting overexpression comprises performing nucleic acid amplification.

116. The method of claim 114, wherein the marker-detectable disease is cancer, and is selected from the group consisting of ovarian cancer, breast cancer, colon cancer, lung cancer and prostate cancer, and wherein the cancer is invasive or metastatic.

117. The method of claim 116, wherein the disease is ovarian cancer and the method further comprises determining one or more of the following: detecting a condition selected from the group consisting of invasive ovarian cancer, metastatic ovarian cancer, a non-ovarian cancer condition elevating serum levels of one or more ovary related markers; identifying a metastasis of unknown origin originating from a primary ovarian cancer, distinguishing between different types of ovarian cancer, differentially diagnosing benign or malignant ovarian cysts; correlating ovarian cancer stage or malignant potential; differentially diagnosing ovarian cancer or a non-ovarian cancer selected from the group consisting of benign (functional) ovarian cyst, uterine fibroids, endometriosis, benign ovarian neoplasms or inflammatory bowel lesions; or determining whether ovarian cancer causes a condition suggestive of a malignant tumor selected from the group consisting of anorexia, cachexia, weight loss, fever, hypercalcemia, skeletal or abdominal pain, paraneoplastic syndrome; or ascites.

118. The method of claim 117, wherein said metastasis of unknown origin comprises one or more of gastric carcinoma, breast cancer, colorectal carcinoma or pancreatic carcinoma.

119. The method of claim 117, wherein said non-ovarian cancer condition elevating serum levels of one or more ovary related markers comprises one or more of cancers of the endometrium, cervix, fallopian tubes, pancreas, breast, lung and colon; pregnancy, endometriosis, pelvic inflammatory disease or uterine fibroids.

120. The method of claim 116, wherein the disease is colon cancer and the method further comprises determining one or more of the following: detecting a condition selected from the group consisting of invasive colon cancer, metastatic colon cancer or colon adenocarcinoma.

121. The method of claim 116, wherein the disease is prostate cancer and the method further comprises determining one or more of the following: detecting a condition selected from the group consisting of invasive prostate cancer or metastatic prostate cancer.

122. The method of claim 116, wherein the disease is breast cancer and the method further comprises determining one or more of the following: determining a likely outcome; detecting breast cancer in patients with age above 55 and/or patients with an age below 45; identifying a metastasis of unknown origin which originated from a primary breast cancer tumor; assessing lymphadenopathy; distinguishing between different types of breast cancer; differentially diagnosing between a benign and malignant breast mass; assessing conditions affecting breast skin for differentiation from breast cancer; differentially diagnosing between breast pain or discomfort resulting from either breast cancer or other conditions; differentially diagnosing between breast cancer and one or more of an abnormal mammogram and/or nipple retraction and/or nipple discharge due to causes other than breast cancer; determining whether breast cancer is causing a condition suggestive of a malignant tumor selected from the group consisting of anorexia, cachexia, weight loss, fever, hypercalcemia, paraneoplastic syndrome; differentially diagnosing between breast cancer and one or more of other malignancies, infections and autoimmune diseases.

123. The method of claim 116, wherein the disease is lung cancer and the method further comprises determining one or more of the following: detecting a condition selected from the group consisting of invasive lung cancer, metastatic lung cancer, squamous cell lung carcinoma, lung adenocarcinoma, carcinoid, small cell lung cancer, non-small cell lung cancer; identifying a metastasis of unknown origin which originated from a primary lung cancer; assessing a malignant tissue residing in the lung that is from a non-lung origin; distinguishing between different types of lung cancer; determining a cause of unexplained dyspnea and/or chronic cough and/or hemoptysis; differentially diagnosing origin of a pleural effusion; differentially diagnosing between lung cancer and a non-lung cancer condition selected from the group consisting of non-malignant causes of lung symptoms and signs; lung lesions and infiltrates, wheeze, stridor, tracheal obstruction, esophageal compression, dysphagia, recurrent laryngeal nerve paralysis, hoarseness, phrenic nerve paralysis with elevation of the hemidiaphragm and Horner syndrome; or determining a cause of a condition suggestive of a malignant tumor selected from the group consisting of anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

124. The method of claim 123, wherein said non-lung origin of said malignant tissue in the lung is selected from the group consisting of osteogenic and soft tissue sarcomas; colorectal, uterine, cervix and corpus tumors; head and neck, breast, testis and salivary gland cancers; melanoma; and bladder and kidney tumors.

125. The method of claim 114, wherein the marker-detectable disease is heart disorder, cardiac disease or cardiac pathology, and is selected from the group consisting of: Myocardial infarction; ungina pectoris (stable and unstable); cardiomyopathy; myocarditis; heart failure; congestive heart failure; the detection of reinfarction; the detection of success of thrombolytic therapy after Myocardial infarct; Myocardial infarct after surgery, or assessing the size of infarct in Myocardial infarct; acute coronary syndrome; the differential diagnosis of heart related conditions from lung related conditions (as pulmonary embolism); the differential diagnosis of Dyspnea; cardiac valves related conditions; sudden cardiac death from arrhythmia or any other heart related reason; rejection of a transplanted heart; atrial and ventricular septal defects; conditions that cause atrial and or ventricular wall volume overload, systemic arterial hypertension.

126. The method of claim 114, wherein the marker-detectable disease is cerebrovascular disease, and is selected from the group consisting of: stroke; ischemic stroke; thrombotic stroke; embolic stroke; lacunar stroke; hypoperfusion stroke; hemorrhagic stroke; transient ischemic attacks; neural tissue injury; brain trauma; and any type of cerebrovascular accident.

127. The method of claim 114, wherein the marker-detectable disease is acute and chronic inflammation is selected from the group consisting of hypercholesterolemia, diabetes, atherosclerosis, acute or chronic inflammation that involves blood vessels, including coronary arteries and blood vessels of the brain, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behçet syndrome, and their complications including coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina; rheumatic/autoimmune diseases that involve systemic immune reaction including rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus; acute or chronic infective processes that involve systemic immune reaction, including pneumonia, bacteremia, sepsis, pyelonephritis, cellulitis, osteomyelitis, meningitis and viral hepatitis; malignant and idiopathic processes that involve systemic immune reaction or proliferation of immune cells, including granulomatous disorders, Wegener's granulomatosis, lymphomatoid granulomatosis/polymorphic reticulosis, idiopathic midline granuloma, multiple myeloma, Waldenstrom's macroglobulinemia, Castleman's disease, histiocytosis.

128. A method for detecting a marker-detectable disease, comprising detecting overexpression of the polypeptide comprising the polypeptide sequence set forth in a member selected from the group consisting of SEQ ID NOs: 165, 166, 317-322, 364-369, 401-404, 427-429, 464-468, 642, 643, 650-664 in a sample from a patient.

129. The method of claim 128, wherein said detecting comprises detecting binding of an antibody to specifically bind to an isolated polynucleotide comprising the polynucleotide sequence set forth in a member selected from the group consisting of SEQ ID NOs: 10, 11, 13, 26, 93, 96, 99, 120, 123, 126, 144, 147, 150-163, 247, 250-315, 325, 328, 331, 334, 337-362, 370, 375, 378, 381, 384, 387-399, 407, 410-425, 432-462, 471, 474, 477, 550, 552, 557, 560, 563, 585, 588, 591, 624, 627, 630 and 698, or a sequence at least about 95% identical thereto, wherein the polypeptide comprising the polypeptide sequence set forth in a member selected from the group consisting of SEQ ID NOs: 165, 166, 317-322, 364-369, 401-404, 427-429, 464-468, 642, 643, 650-664 in a sample from a patient.

130. The method of claim 128 wherein the marker-detectable disease is cancer, and is selected from the group consisting of ovarian cancer, breast cancer, colon cancer, lung cancer and prostate cancer, and wherein the cancer is invasive or metastatic.

131. The method of claim 130, wherein the disease is ovarian cancer and the method further comprises determining one or more of the following: detecting a condition selected from the group consisting of invasive ovarian cancer, metastatic ovarian cancer, a non-ovarian cancer condition elevating serum levels of one or more ovary related markers; identifying a metastasis of unknown origin originating from a primary ovarian cancer, distinguishing between different types of ovarian cancer, differentially diagnosing benign or malignant ovarian cysts; correlating ovarian cancer stage and/or malignant potential; differentially diagnosing ovarian cancer or a non-ovarian cancer selected from the group consisting of benign (functional) ovarian cyst, uterine fibroids, endometriosis, benign ovarian neoplasms or inflammatory bowel lesions; or determining whether ovarian cancer causes a condition suggestive of a malignant tumor selected from the group consisting of anorexia, cachexia, weight loss, fever, hypercalcemia, skeletal or abdominal pain, paraneoplastic syndrome; or ascites.

132. The method of claim 131, wherein said metastasis of unknown origin comprises one or more of gastric carcinoma, breast cancer, colorectal carcinoma or pancreatic carcinoma.

133. The method of claim 132, wherein said non-ovarian cancer condition elevating serum levels of one or more ovary related markers comprises one or more of cancers of the endometrium, cervix, fallopian tubes, pancreas, breast, lung and colon; pregnancy, endometriosis, pelvic inflammatory disease or uterine fibroids.

134. The method of claim 128, wherein the disease is colon cancer and the method further comprises determining one or more of the following: detecting a condition selected from the group consisting of invasive colon cancer, metastatic colon cancer or colon adenocarcinoma.

135. The method of claim 128, wherein the disease is prostate cancer and the method further comprises determining one or more of the following: detecting a condition selected from the group consisting of invasive prostate cancer or metastatic prostate cancer.

136. The method of claim 128, wherein the disease is breast cancer and the method further comprises determining one or more of the following: determining a likely outcome; detecting breast cancer in patients with age above 55 and/or patients with an age below 45; identifying a metastasis of unknown origin which originated from a primary breast cancer tumor; assessing lymphadenopathy; distinguishing between different types of breast cancer; differentially diagnosing between a benign and malignant breast mass; assessing conditions affecting breast skin for differentiation from breast cancer; differentially diagnosing between breast pain or discomfort resulting from either breast cancer or other conditions; differentially diagnosing between breast cancer and one or more of an abnormal mammogram and/or nipple retraction and/or nipple discharge due to causes other than breast cancer; determining whether breast cancer is causing a condition suggestive of a malignant tumor selected from the group consisting of anorexia, cachexia, weight loss, fever, hypercalcemia, paraneoplastic syndrome; differentially diagnosing between breast cancer and one or more of other malignancies, infections and autoimmune diseases.

137. The method of claim 128, wherein the disease is lung cancer and the method further comprises determining one or more of the following: detecting a condition selected from the group consisting of invasive lung cancer, metastatic lung cancer, squamous cell lung carcinoma, lung adenocarcinoma, carcinoid, small cell lung cancer, non-small cell lung cancer; identifying a metastasis of unknown origin which originated from a primary lung cancer; assessing a malignant tissue residing in the lung that is from a non-lung origin; distinguishing between different types of lung cancer; determining a cause of unexplained dyspnea and/or chronic cough and/or hemoptysis; differentially diagnosing origin of a pleural effusion; differentially diagnosing between lung cancer and a non-lung cancer condition selected from the group consisting of non-malignant causes of lung symptoms and signs; lung lesions and infiltrates, wheeze, stridor, tracheal obstruction, esophageal compression, dysphagia, recurrent laryngeal nerve paralysis, hoarseness, phrenic nerve paralysis with elevation of the hemidiaphragm and Horner syndrome; or determining a cause of a condition suggestive of a malignant tumor selected from the group consisting of anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

138. The method of claim 137, wherein said non-lung origin of said malignant tissue in the lung is selected from the group consisting of osteogenic and soft tissue sarcomas; colorectal, uterine, cervix and corpus tumors; head and neck, breast, testis and salivary gland cancers; melanoma; and bladder and kidney tumors.

139. The method of claim 128, wherein the marker-detectable disease is heart disorder, cardiac disease or cardiac pathology, and is selected from the group consisting of: Myocardial infarction; ungina pectoris (stable and unstable); cardiomyopathy; myocarditis; heart failure; congestive heart failure; the detection of reinfarction; the detection of success of thrombolytic therapy after Myocardial infarct; Myocardial infarct after surgery, or assessing the size of infarct in Myocardial infarct; acute coronary syndrome; the differential diagnosis of heart related conditions from lung related conditions (as pulmonary embolism); the differential diagnosis of Dyspnea; cardiac valves related conditions; sudden cardiac death from arrhythmia or any other heart related reason; rejection of a transplanted heart; atrial and ventricular septal defects; conditions that cause atrial and or ventricular wall volume overload, systemic arterial hypertension.

140. The method of claim 128, wherein the marker-detectable disease is cerebrovascular disease, and is selected from the group consisting of: stroke; ischemic stroke; thrombotic stroke; embolic stroke; lacunar stroke; hypoperfusion stroke; hemorrhagic stroke; transient ischemic attacks; neural tissue injury; brain trauma; and any type of cerebrovascular accident.

141. The method of claim 128, wherein the marker-detectable disease is acute and chronic inflammation is selected from the group consisting of hypercholesterolemia, diabetes, atherosclerosis, acute or chronic inflammation that involves blood vessels, including coronary arteries and blood vessels of the brain, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behçet syndrome, and their complications including coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina; rheumatic/autoimmune diseases that involve systemic immune reaction including rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus; acute or chronic infective processes that involve systemic immune reaction, including pneumonia, bacteremia, sepsis, pyelonephritis, cellulitis, osteomyelitis, meningitis and viral hepatitis; malignant and idiopathic processes that involve systemic immune reaction or proliferation of immune cells, including granulomatous disorders, Wegener's granulomatosis, lymphomatoid granulomatosis/polymorphic reticulosis, idiopathic midline granuloma, multiple myeloma, Waldenstrom's macroglobulinemia, Castleman's disease, histiocytosis.

142. A biomarker for detecting a marker-detectable disease, comprising an amino acid sequence of claim 95, marked with a label.

143. A method to screen for or to diagnose a marker-detectable disease, comprising detecting the disease with the biomarker of claim 142.

144. A method for monitoring disease progression, treatment efficacy or relapse of a marker-detectable disease, comprising detecting the disease with the biomarker of claim 142.

145. A method of selecting a therapy for a marker-detectable disease, comprising detecting the disease with the biomarker of claim 142 and selecting a therapy according to said detection.

146. A biomarker for detecting a marker-detectable disease, comprising a nucleotide acid sequence set forth in a member selected from the group consisting of SEQ ID NOs: 10, 11, 13, 26, 93, 96, 99, 120, 123, 126, 144, 147, 150-163, 247, 250-315, 325, 328, 331, 334, 337-362, 370, 375, 378, 381, 384, 387-399, 407, 410-425, 432-462, 471, 474, 477, 550, 552, 557, 560, 563, 585, 588, 591, 624, 627, 630 and 698, or a sequence at least about 95% identical thereto.

147. A method to screen for or to diagnose a marker-detectable disease, comprising detecting the disease with the biomarker of claim 146.

148. A method for monitoring disease progression, treatment efficacy or relapse of a marker-detectable disease, comprising detecting the disease with the biomarker of claim 146.

149. A method of selecting a therapy for a marker-detectable disease, comprising detecting the disease with the biomarker of claim 146 and selecting a therapy according to said detection.
